(19) United States
(12) Patent Application Publication
(10) Pub. No.: US 2026/0034230 A1
(43) Pub. Date: Feb. 5, 2026

(54) COMPOSITIONS AND METHODS FOR TRANSFERRIN RECEPTOR (TFR)-MEDIATED DELIVERY TO THE BRAIN AND MUSCLE

(71) Applicant: Regeneron Pharmaceuticals, Inc., Tarrytown, NY (US)

(72) Inventors: Bojie ZHANG, White Plains, NY (US); Nicole KEATING, Yorktown Heights, NY (US); Pascaline AIMÉ-WILSON, New York, NY (US); John DUGAN, Hoboken, NJ (US); Min GAO, Woodcliff Lake, NJ (US); Robert BABB, River Edge, NJ (US); Maria PRAGGASTIS, Cortlandt Manor, NY (US); Katherine CYGNAR, White Plains, NY (US)

(73) Assignee: Regeneron Pharmaceuticals, Inc., Tarrytown, NY (US)

(21) Appl. No.: 19/099,243

(22) PCT Filed: Jul. 28, 2023
(86) PCT No.: PCT/US23/71243

§ 371 Date: Jan. 28, 2025

Related U.S. Application Data
(60) Provisional application No. 63/393,749, filed on Jul. 29, 2022.

(51) Int. Cl. A61K 47/68; C12N 9/22; C12N 15/11; C12N 15/88

(52) U.S. Cl. CPC A61K 47/6849; A61K 47/6807; C12N 9/226; C12N 15/11; C12N 15/88; C12N 2310/20

(57) ABSTRACT

The present invention provides, in part, protein-drug conjugates comprising an anti-transferrin receptor (e.g., human transferrin receptor) antigen-binding protein (e.g., scFv, Fab) conjugated to a molecular cargo (e.g., polynucleotides, liposomes or lipid nanoparticles) for delivery of the molecular cargo to a targeted tissue (e.g., brain or muscle). Methods for treating various diseases or disorders, such as neurological diseases or muscular diseases, with the conjugates are provided.

### CROSS REFERENCE TO RELATED APPLICATIONS

This application claims priority to U.S. Provisional Application No. 63/393,749, filed Jul. 29, 2022, which is hereby incorporated by reference in its entirety.

### SEQUENCE LISTING

The instant application contains a Sequence Listing which has been submitted electronically in XML file format and is hereby incorporated by reference in its entirety. Said XML copy, created on Jul. 26, 2023, is named 250298000507SEQLIST.XML and is 547,729 bytes in size.

### FIELD OF THE INVENTION

The present invention relates to protein-drug conjugates including an antigen-binding protein conjugated to a molecular cargo, as well as method of treating diseases with such protein-drug conjugates.

### BACKGROUND OF THE INVENTION

Iron delivery to the brain is accomplished via binding and intracellular trafficking of the iron binding protein transferrin (Tf). The Tf receptor (TfR) is a target of some studies to deliver drugs to the brain. For example, approaches include the use of liposomes decorated with Tf used for delivery of imaging agents and DNA (Sharma et al., (2013) Cell penetrating peptide tethered bi-ligand liposomes for delivery to brain in vivo: biodistribution and transfection. J. Control. Release 167, 1-10.) or the use of an iron-mimetic peptide as ligand (Staquicini et al., (2011)).

A correlation has also been suggested between increased antibody affinity and lysosomal degradation (Bien-Ly et al., (2014) Transferrin receptor (TfR) trafficking determines brain uptake of TfR antibody affinity variants. J. Exp. Med. 211, 233-244) supporting the idea that lower antibody's affinity would help avoid intracellular degradation of the complexes being transported. Bien-Ly et al. found that bispecific antibodies against TfR and beta-secretase (BACE1) traversed the blood-brain barrier (BBB) and effectively reduce brain amyloid beta levels, but also that high-affinity binding to TfR caused a dose-dependent reduction of brain TfR levels. Similarly, Moos & Morgan (2001) compared the ability of anti-TfR antibody, OX26, and transferrin to cross the rat BBB finding that OX26 did not recycle out of the brain as did transferrin because the antibody exhibited a high-affinity antibody-antigen interaction with TfR that is not easily reversed, whereas that of Tf is readily reversed depending on pH and the iron content of Tf (Restricted transport of anti-transferrin receptor antibody (OX26) through the blood-brain barrier in the rat, J Neurochem 2001 October; 79(1):119-29).

### SUMMARY OF THE INVENTION

In one aspect, provided herein is a protein-drug conjugate comprising an antigen-binding protein that binds specifically to human transferrin receptor (TfR) or a variant or an antigenic fragment thereof, which is conjugated to a molecular cargo.

The antigen-binding protein may bind to human transferrin receptor with a K_{D} of about 41 nM or a stronger affinity, e.g., about 30 nM or stronger affinity, about 20 nM or stronger affinity, about 10 nM or stronger affinity, about 5 nM or stronger affinity, about 3 nM or stronger affinity, or about 1 nM or stronger affinity. In some embodiments, the antigen-binding protein binds to human transferrin receptor with a K_{D} of about 3 nM or a stronger affinity. In some embodiments, the antigen-binding protein binds to human transferrin receptor with a K_{D} of about 0.45 nM to 3 nM. Such binding affinity may be measured in a surface plasmon resonance assay at, for example, 25° C.

In some embodiments, the antigen-binding protein may comprise a heavy chain variable region (HCVR or V_{H}) and a light chain variable region (LCVR or V_{L}), and wherein a Fab having said HCVR and LCVR binds to human transferrin receptor with a K_{D} of about 0.65 nM or a stronger affinity.

In some embodiments, the antigen binding protein comprises an antibody or antigen-binding fragment thereof. The antigen-binding fragment can be selected from a humanized antibody or antigen binding fragment thereof, human antibody or antigen binding fragment thereof, murine antibody or antigen binding fragment thereof, chimeric antibody or antigen binding fragment thereof, monovalent Fab′, divalent Fab2, F(ab)′3 fragments, single-chain fragment variable (scFv), bis-scFv, (scFv)2, diabody, bivalent antibody, one-armed antibody, minibody, nanobody, triabody, tetrabody, disulfide stabilized Fv protein (dsFv), single-domain antibody (sdAb), Ig NAR, single heavy chain antibody, bispecific antibody or binding fragment thereof, bi-specific T-cell engager (BiTE), trispecific antibody, or chemically modified derivatives thereof.

In some embodiments, the antigen-binding protein of the protein-drug conjugate comprises an antigen-binding fragment that is a fragment antigen-binding region (Fab).

In some embodiments, the antigen-binding protein of the protein-drug conjugate comprises a single chain fragment variable (scFv). In some embodiments, the protein-drug conjugate a single chain fragment variable (scFv) comprising domains arranged in the following orientation from N-terminus to C-terminus: Heavy chain variable region (HCVR)-Light chain variable region (LCVR). In some embodiments, the protein-drug conjugate comprises a single chain fragment variable (scFv) comprising domains arranged in the following orientation from N-terminus to C-terminus: Light chain variable region (LCVR)-Heavy chain variable region (HCVR). In some embodiments, said scFv variable regions are connected by a peptide linker. In some embodiments, said scFv variable regions are connected by a peptide linker which is -(GGGGS)ₙ- (SEQ ID NO: 426); wherein n is 1, 2, 3, 4, 5, 6, 7, 8, 9 or 10. In some embodiments, the antigen-binding protein of the protein-drug conjugate binds to human transferrin receptor with a K_{D} of about 1×10⁻⁷ M or a stronger affinity.

In some embodiments, the antigen-binding protein of the protein-drug conjugate comprises: (i) a HCVR that comprises the HCDR1, HCDR2 and HCDR3 of a HCVR comprising the amino acid sequence set forth in SEQ ID NO: 2; 462; 12; 463; 22; 464; 32; 42; 52; 467; 62; 492; 72; 470; 82; 92; 472; 102; 112; 473; 122; 132; 142; 475; 152; 162; 477; 172; 182; 478; 192; 480; 202; 481; 212; 222; 232; 242; 252; 482; 262; 272; 282; 292; 302; 483 or 312 (or a variant thereof); and/or (ii) a LCVR that comprises the LCDR1, LCDR2 and LCDR3 of a LCVR comprising the amino acid sequence set forth in SEQ ID NO: 7; 17; 27; 37; 465; 47; 466; 57; 468; 67; 469; 77; 471; 87; 97; 107; 117; 474; 127; 137; 147; 476; 157; 167; 177; 187; 479; 197; 207; 217; 227; 237; 247; 257; 267; 277; 287; 297; 307; 527; 317 or 484 (or a variant thereof).

In some embodiments, the antigen-binding protein of the protein-drug conjugate comprises: (1) a HCVR comprising the HCDR1, HCDR2 and HCDR3 of a HCVR that comprises the amino acid sequence set forth in SEQ ID NO: 2 (or a variant thereof); and a LCVR comprising the LCDR1, LCDR2 and LCDR3 of a LCVR that comprises the amino acid sequence set forth in SEQ ID NO: 7 (or a variant thereof); (2) a HCVR comprising the HCDR1, HCDR2 and HCDR3 of a HCVR that comprises the amino acid sequence set forth in SEQ ID NO: 12 (or a variant thereof); and a LCVR comprising the LCDR1, LCDR2 and LCDR3 of a LCVR that comprises the amino acid sequence set forth in SEQ ID NO: 17 (or a variant thereof); (3) a HCVR comprising the HCDR1, HCDR2 and HCDR3 of a HCVR that comprises the amino acid sequence set forth in SEQ ID NO: 22 (or a variant thereof); and a LCVR comprising the LCDR1, LCDR2 and LCDR3 of a LCVR that comprises the amino acid sequence set forth in SEQ ID NO: 27 (or a variant thereof); (4) a HCVR comprising the HCDR1, HCDR2 and HCDR3 of a HCVR that comprises the amino acid sequence set forth in SEQ ID NO: 32 (or a variant thereof); and a LCVR comprising the LCDR1, LCDR2 and LCDR3 of a LCVR that comprises the amino acid sequence set forth in SEQ ID NO: 37 (or a variant thereof); (5) a HCVR comprising the HCDR1, HCDR2 and HCDR3 of a HCVR that comprises the amino acid sequence set forth in SEQ ID NO: 42 (or a variant thereof); and a LCVR comprising the LCDR1, LCDR2 and LCDR3 of a LCVR that comprises the amino acid sequence set forth in SEQ ID NO: 47 (or a variant thereof); (6) a HCVR comprising the HCDR1, HCDR2 and HCDR3 of a HCVR that comprises the amino acid sequence set forth in SEQ ID NO: 52 (or a variant thereof); and a LCVR comprising the LCDR1, LCDR2 and LCDR3 of a LCVR that comprises the amino acid sequence set forth in SEQ ID NO: 57 (or a variant thereof); (7) a HCVR comprising the HCDR1, HCDR2 and HCDR3 of a HCVR that comprises the amino acid sequence set forth in SEQ ID NO: 62 (or a variant thereof); and a LCVR comprising the LCDR1, LCDR2 and LCDR3 of a LCVR that comprises the amino acid sequence set forth in SEQ ID NO: 67 (or a variant thereof); (8) a HCVR comprising the HCDR1, HCDR2 and HCDR3 of a HCVR that comprises the amino acid sequence set forth in SEQ ID NO: 72 (or a variant thereof); and a LCVR comprising the LCDR1, LCDR2 and LCDR3 of a LCVR that comprises the amino acid sequence set forth in SEQ ID NO: 77 (or a variant thereof); (9) a HCVR comprising the HCDR1, HCDR2 and HCDR3 of a HCVR that comprises the amino acid sequence set forth in SEQ ID NO: 82 (or a variant thereof); and a LCVR comprising the LCDR1, LCDR2 and LCDR3 of a LCVR that comprises the amino acid sequence set forth in SEQ ID NO: 87 (or a variant thereof); (10) a HCVR comprising the HCDR1, HCDR2 and HCDR3 of a HCVR that comprises the amino acid sequence set forth in SEQ ID NO: 92 (or a variant thereof); and a LCVR comprising the LCDR1, LCDR2 and LCDR3 of a LCVR that comprises the amino acid sequence set forth in SEQ ID NO: 97 (or a variant thereof); (11) a HCVR comprising the HCDR1, HCDR2 and HCDR3 of a HCVR that comprises the amino acid sequence set forth in SEQ ID NO: 102 (or a variant thereof); and a LCVR comprising the LCDR1, LCDR2 and LCDR3 of a LCVR that comprises the amino acid sequence set forth in SEQ ID NO: 107 (or a variant thereof); (12) a HCVR comprising the HCDR1, HCDR2 and HCDR3 of a HCVR that comprises the amino acid sequence set forth in SEQ ID NO: 112 (or a variant thereof); and a LCVR comprising the LCDR1, LCDR2 and LCDR3 of a LCVR that comprises the amino acid sequence set forth in SEQ ID NO: 117 (or a variant thereof); (13) a HCVR comprising the HCDR1, HCDR2 and HCDR3 of a HCVR that comprises the amino acid sequence set forth in SEQ ID NO: 122 (or a variant thereof); and a LCVR comprising the LCDR1, LCDR2 and LCDR3 of a LCVR that comprises the amino acid sequence set forth in SEQ ID NO: 127 (or a variant thereof); (14) a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof); (15) a HCVR comprising the HCDR1, HCDR2 and HCDR3 of a HCVR that comprises the amino acid sequence set forth in SEQ ID NO: 142 (or a variant thereof); and a LCVR comprising the LCDR1, LCDR2 and LCDR3 of a LCVR that comprises the amino acid sequence set forth in SEQ ID NO: 147 (or a variant thereof); (16) a HCVR comprising the HCDR1, HCDR2 and HCDR3 of a HCVR that comprises the amino acid sequence set forth in SEQ ID NO: 152 (or a variant thereof); and a LCVR comprising the LCDR1, LCDR2 and LCDR3 of a LCVR that comprises the amino acid sequence set forth in SEQ ID NO: 157 (or a variant thereof); (17) a HCVR comprising the HCDR1, HCDR2 and HCDR3 of a HCVR that comprises the amino acid sequence set forth in SEQ ID NO: 162 (or a variant thereof); and a LCVR comprising the LCDR1, LCDR2 and LCDR3 of a LCVR that comprises the amino acid sequence set forth in SEQ ID NO: 167 (or a variant thereof); (18) a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof); (19) a HCVR comprising the HCDR1, HCDR2 and HCDR3 of a HCVR that comprises the amino acid sequence set forth in SEQ ID NO: 182 (or a variant thereof); and a LCVR comprising the LCDR1, LCDR2 and LCDR3 of a LCVR that comprises the amino acid sequence set forth in SEQ ID NO: 187 (or a variant thereof); (20) a HCVR comprising the HCDR1, HCDR2 and HCDR3 of a HCVR that comprises the amino acid sequence set forth in SEQ ID NO: 192 (or a variant thereof); and a LCVR comprising the LCDR1, LCDR2 and LCDR3 of a LCVR that comprises the amino acid sequence set forth in SEQ ID NO: 197 (or a variant thereof); (21) a HCVR comprising the HCDR1, HCDR2 and HCDR3 of a HCVR that comprises the amino acid sequence set forth in SEQ ID NO: 202 (or a variant thereof); and a LCVR comprising the LCDR1, LCDR2 and LCDR3 of a LCVR that comprises the amino acid sequence set forth in SEQ ID NO: 207 (or a variant thereof); (22) a HCVR comprising the HCDR1, HCDR2 and HCDR3 of a HCVR that comprises the amino acid sequence set forth in SEQ ID NO: 212 (or a variant thereof); and a LCVR comprising the LCDR1, LCDR2 and LCDR3 of a LCVR that comprises the amino acid sequence set forth in SEQ ID NO: 217 (or a variant thereof); (23) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof); (24) a HCVR comprising the HCDR1, HCDR2 and HCDR3 of a HCVR that comprises the amino acid sequence set forth in SEQ ID NO: 232 (or a variant thereof); and a LCVR comprising the LCDR1, LCDR2 and LCDR3 of a LCVR that comprises the amino acid sequence set forth in SEQ ID NO: 237 (or a variant thereof); (25) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof); (26) a HCVR comprising the HCDR1, HCDR2 and HCDR3 of a HCVR that comprises the amino acid sequence set forth in SEQ ID NO: 252 (or a variant thereof); and a LCVR comprising the LCDR1, LCDR2 and LCDR3 of a LCVR that comprises the amino acid sequence set forth in SEQ ID NO: 257 (or a variant thereof); (27) a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof); (28) a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof); (29) a HCVR comprising the HCDR1, HCDR2 and HCDR3 of a HCVR that comprises the amino acid sequence set forth in SEQ ID NO: 282 (or a variant thereof); and a LCVR comprising the LCDR1, LCDR2 and LCDR3 of a LCVR that comprises the amino acid sequence set forth in SEQ ID NO: 287 (or a variant thereof); (30) a HCVR comprising the HCDR1, HCDR2 and HCDR3 of a HCVR that comprises the amino acid sequence set forth in SEQ ID NO: 292 (or a variant thereof); and a LCVR comprising the LCDR1, LCDR2 and LCDR3 of a LCVR that comprises the amino acid sequence set forth in SEQ ID NO: 297 (or a variant thereof); (31) a HCVR comprising the HCDR1, HCDR2 and HCDR3 of a HCVR that comprises the amino acid sequence set forth in SEQ ID NO: 302 (or a variant thereof); and a LCVR comprising the LCDR1, LCDR2 and LCDR3 of a LCVR that comprises the amino acid sequence set forth in SEQ ID NO: 307 or 527 (or a variant thereof); and/or (32) a HCVR comprising the HCDR1, HCDR2 and HCDR3 of a HCVR that comprises the amino acid sequence set forth in SEQ ID NO: 312 (or a variant thereof); and a LCVR comprising the LCDR1, LCDR2 and LCDR3 of a LCVR that comprises the amino acid sequence set forth in SEQ ID NO: 317 (or a variant thereof).

In some embodiments, the antigen-binding protein of the protein-drug conjugate comprises: (a) a HCVR that comprises: an HCDR1 comprising the amino acid sequence set forth in SEQ ID NO: 3 (or a variant thereof), an HCDR2 comprising the amino acid sequence set forth in SEQ ID NO: 4 (or a variant thereof), and an HCDR3 comprising the amino acid sequence set forth in SEQ ID NO: 5 (or a variant thereof); and a LCVR that comprises: an LCDR1 comprising the amino acid sequence set forth in SEQ ID NO: 8 (or a variant thereof), an LCDR2 comprising the amino acid sequence set forth in SEQ ID NO: 9 (or a variant thereof), and an LCDR3 comprising the amino acid sequence set forth in SEQ ID NO: 10 (or a variant thereof); (b) a HCVR that comprises: an HCDR1 comprising the amino acid sequence set forth in SEQ ID NO: 13 (or a variant thereof), an HCDR2 comprising the amino acid sequence set forth in SEQ ID NO: 14 (or a variant thereof), and an HCDR3 comprising the amino acid sequence set forth in SEQ ID NO: 15 (or a variant thereof); and a LCVR that comprises: an LCDR1 comprising the amino acid sequence set forth in SEQ ID NO: 18 (or a variant thereof), an LCDR2 comprising the amino acid sequence set forth in SEQ ID NO: 19 (or a variant thereof), and an LCDR3 comprising the amino acid sequence set forth in SEQ ID NO: 20 (or a variant thereof); (c) a HCVR that comprises: an HCDR1 comprising the amino acid sequence set forth in SEQ ID NO: 23 (or a variant thereof), an HCDR2 comprising the amino acid sequence set forth in SEQ ID NO: 24 (or a variant thereof), and an HCDR3 comprising the amino acid sequence set forth in SEQ ID NO: 25 (or a variant thereof); and a LCVR that comprises: an LCDR1 comprising the amino acid sequence set forth in SEQ ID NO: 28 (or a variant thereof), an LCDR2 comprising the amino acid sequence set forth in SEQ ID NO: 29 (or a variant thereof), and an LCDR3 comprising the amino acid sequence set forth in SEQ ID NO: 30 (or a variant thereof); (d) a HCVR that comprises: an HCDR1 comprising the amino acid sequence set forth in SEQ ID NO: 33 (or a variant thereof), an HCDR2 comprising the amino acid sequence set forth in SEQ ID NO: 34 (or a variant thereof), and an HCDR3 comprising the amino acid sequence set forth in SEQ ID NO: 35 (or a variant thereof); and a LCVR that comprises: an LCDR1 comprising the amino acid sequence set forth in SEQ ID NO: 38 (or a variant thereof), an LCDR2 comprising the amino acid sequence set forth in SEQ ID NO: 39 (or a variant thereof), and an LCDR3 comprising the amino acid sequence set forth in SEQ ID NO: 40 (or a variant thereof); (e) a HCVR that comprises: an HCDR1 comprising the amino acid sequence set forth in SEQ ID NO: 43 (or a variant thereof), an HCDR2 comprising the amino acid sequence set forth in SEQ ID NO: 44 (or a variant thereof), and an HCDR3 comprising the amino acid sequence set forth in SEQ ID NO: 45 (or a variant thereof); and a LCVR that comprises: an LCDR1 comprising the amino acid sequence set forth in SEQ ID NO: 48 (or a variant thereof), an LCDR2 comprising the amino acid sequence set forth in SEQ ID NO: 49 (or a variant thereof), and an LCDR3 comprising the amino acid sequence set forth in SEQ ID NO: 50 (or a variant thereof); (f) a HCVR that comprises: an HCDR1 comprising the amino acid sequence set forth in SEQ ID NO: 53 (or a variant thereof), an HCDR2 comprising the amino acid sequence set forth in SEQ ID NO: 54 (or a variant thereof), and an HCDR3 comprising the amino acid sequence set forth in SEQ ID NO: 55 (or a variant thereof); and a LCVR that comprises: an LCDR1 comprising the amino acid sequence set forth in SEQ ID NO: 58 (or a variant thereof), an LCDR2 comprising the amino acid sequence set forth in SEQ ID NO: 59 (or a variant thereof), and an LCDR3 comprising the amino acid sequence set forth in SEQ ID NO: 60 (or a variant thereof); (g) a HCVR that comprises: an HCDR1 comprising the amino acid sequence set forth in SEQ ID NO: 63 (or a variant thereof), an HCDR2 comprising the amino acid sequence set forth in SEQ ID NO: 64 (or a variant thereof), and an HCDR3 comprising the amino acid sequence set forth in SEQ ID NO: 65 (or a variant thereof); and a LCVR that comprises: an LCDR1 comprising the amino acid sequence set forth in SEQ ID NO: 68 (or a variant thereof), an LCDR2 comprising the amino acid sequence set forth in SEQ ID NO: 69 (or a variant thereof), and an LCDR3 comprising the amino acid sequence set forth in SEQ ID NO: 70 (or a variant thereof); (h) a HCVR that comprises: an HCDR1 comprising the amino acid sequence set forth in SEQ ID NO: 73 (or a variant thereof), an HCDR2 comprising the amino acid sequence set forth in SEQ ID NO: 74 (or a variant thereof), and an HCDR3 comprising the amino acid sequence set forth in SEQ ID NO: 75 (or a variant thereof); and a LCVR that comprises: an LCDR1 comprising the amino acid sequence set forth in SEQ ID NO: 78 (or a variant thereof), an LCDR2 comprising the amino acid sequence set forth in SEQ ID NO: 79 (or a variant thereof), and an LCDR3 comprising the amino acid sequence set forth in SEQ ID NO: 80 (or a variant thereof); (i) a HCVR that comprises: an HCDR1 comprising the amino acid sequence set forth in SEQ ID NO: 83 (or a variant thereof), an HCDR2 comprising the amino acid sequence set forth in SEQ ID NO: 84 (or a variant thereof), and an HCDR3 comprising the amino acid sequence set forth in SEQ ID NO: 85 (or a variant thereof); and a LCVR that comprises: an LCDR1 comprising the amino acid sequence set forth in SEQ ID NO: 88 (or a variant thereof), an LCDR2 comprising the amino acid sequence set forth in SEQ ID NO: 89 (or a variant thereof), and an LCDR3 comprising the amino acid sequence set forth in SEQ ID NO: 90 (or a variant thereof); (j) a HCVR that comprises: an HCDR1 comprising the amino acid sequence set forth in SEQ ID NO: 93 (or a variant thereof), an HCDR2 comprising the amino acid sequence set forth in SEQ ID NO: 94 (or a variant thereof), and an HCDR3 comprising the amino acid sequence set forth in SEQ ID NO: 95 (or a variant thereof); and a LCVR that comprises: an LCDR1 comprising the amino acid sequence set forth in SEQ ID NO: 98 (or a variant thereof), an LCDR2 comprising the amino acid sequence set forth in SEQ ID NO: 99 (or a variant thereof), and an LCDR3 comprising the amino acid sequence set forth in SEQ ID NO: 100 (or a variant thereof); (k) a HCVR that comprises: an HCDR1 comprising the amino acid sequence set forth in SEQ ID NO: 103 (or a variant thereof), an HCDR2 comprising the amino acid sequence set forth in SEQ ID NO: 104 (or a variant thereof), and an HCDR3 comprising the amino acid sequence set forth in SEQ ID NO: 105 (or a variant thereof); and a LCVR that comprises: an LCDR1 comprising the amino acid sequence set forth in SEQ ID NO: 108 (or a variant thereof), an LCDR2 comprising the amino acid sequence set forth in SEQ ID NO: 109 (or a variant thereof), and an LCDR3 comprising the amino acid sequence set forth in SEQ ID NO: 110 (or a variant thereof); (I) a HCVR that comprises: an HCDR1 comprising the amino acid sequence set forth in SEQ ID NO: 113 (or a variant thereof), an HCDR2 comprising the amino acid sequence set forth in SEQ ID NO: 114 (or a variant thereof), and an HCDR3 comprising the amino acid sequence set forth in SEQ ID NO: 115 (or a variant thereof); and a LCVR that comprises: an LCDR1 comprising the amino acid sequence set forth in SEQ ID NO: 118 (or a variant thereof), an LCDR2 comprising the amino acid sequence set forth in SEQ ID NO: 119 (or a variant thereof), and an LCDR3 comprising the amino acid sequence set forth in SEQ ID NO: 120 (or a variant thereof); (m) a HCVR that comprises: an HCDR1 comprising the amino acid sequence set forth in SEQ ID NO: 123 (or a variant thereof), an HCDR2 comprising the amino acid sequence set forth in SEQ ID NO: 124 (or a variant thereof), and an HCDR3 comprising the amino acid sequence set forth in SEQ ID NO: 125 (or a variant thereof); and a LCVR that comprises: an LCDR1 comprising the amino acid sequence set forth in SEQ ID NO: 128 (or a variant thereof), an LCDR2 comprising the amino acid sequence set forth in SEQ ID NO: 129 (or a variant thereof), and an LCDR3 comprising the amino acid sequence set forth in SEQ ID NO: 130 (or a variant thereof); (n) a HCVR that comprises: an HCDR1 comprising the amino acid sequence set forth in SEQ ID NO: 133 (or a variant thereof), an HCDR2 comprising the amino acid sequence set forth in SEQ ID NO: 134 (or a variant thereof), and an HCDR3 comprising the amino acid sequence set forth in SEQ ID NO: 135 (or a variant thereof); and a LCVR that comprises: an LCDR1 comprising the amino acid sequence set forth in SEQ ID NO: 138 (or a variant thereof), an LCDR2 comprising the amino acid sequence set forth in SEQ ID NO: 139 (or a variant thereof), and an LCDR3 comprising the amino acid sequence set forth in SEQ ID NO: 140 (or a variant thereof); (o) a HCVR that comprises: an HCDR1 comprising the amino acid sequence set forth in SEQ ID NO: 143 (or a variant thereof), an HCDR2 comprising the amino acid sequence set forth in SEQ ID NO: 144 (or a variant thereof), and an HCDR3 comprising the amino acid sequence set forth in SEQ ID NO: 145 (or a variant thereof); and a LCVR that comprises: an LCDR1 comprising the amino acid sequence set forth in SEQ ID NO: 148 (or a variant thereof), an LCDR2 comprising the amino acid sequence set forth in SEQ ID NO: 149 (or a variant thereof), and an LCDR3 comprising the amino acid sequence set forth in SEQ ID NO: 150 (or a variant thereof); (p) a HCVR that comprises: an HCDR1 comprising the amino acid sequence set forth in SEQ ID NO: 153 (or a variant thereof), an HCDR2 comprising the amino acid sequence set forth in SEQ ID NO: 154 (or a variant thereof), and an HCDR3 comprising the amino acid sequence set forth in SEQ ID NO: 155 (or a variant thereof); and a LCVR that comprises: an LCDR1 comprising the amino acid sequence set forth in SEQ ID NO: 158 (or a variant thereof), an LCDR2 comprising the amino acid sequence set forth in SEQ ID NO: 159 (or a variant thereof), and an LCDR3 comprising the amino acid sequence set forth in SEQ ID NO: 160 (or a variant thereof); (q) a HCVR that comprises: an HCDR1 comprising the amino acid sequence set forth in SEQ ID NO: 163 (or a variant thereof), an HCDR2 comprising the amino acid sequence set forth in SEQ ID NO: 164 (or a variant thereof), and an HCDR3 comprising the amino acid sequence set forth in SEQ ID NO: 165 (or a variant thereof); and a LCVR that comprises: an LCDR1 comprising the amino acid sequence set forth in SEQ ID NO: 168 (or a variant thereof), an LCDR2 comprising the amino acid sequence set forth in SEQ ID NO: 169 (or a variant thereof), and an LCDR3 comprising the amino acid sequence set forth in SEQ ID NO: 170 (or a variant thereof); (r) a HCVR that comprises: an HCDR1 comprising the amino acid sequence set forth in SEQ ID NO: 173 (or a variant thereof), an HCDR2 comprising the amino acid sequence set forth in SEQ ID NO: 174 (or a variant thereof), and an HCDR3 comprising the amino acid sequence set forth in SEQ ID NO: 175 (or a variant thereof); and a LCVR that comprises: an LCDR1 comprising the amino acid sequence set forth in SEQ ID NO: 178 (or a variant thereof), an LCDR2 comprising the amino acid sequence set forth in SEQ ID NO: 179 (or a variant thereof), and an LCDR3 comprising the amino acid sequence set forth in SEQ ID NO: 180 (or a variant thereof); (s) a HCVR that comprises: an HCDR1 comprising the amino acid sequence set forth in SEQ ID NO: 183 (or a variant thereof), an HCDR2 comprising the amino acid sequence set forth in SEQ ID NO: 184 (or a variant thereof), and an HCDR3 comprising the amino acid sequence set forth in SEQ ID NO: 185 (or a variant thereof); and a LCVR that comprises: an LCDR1 comprising the amino acid sequence set forth in SEQ ID NO: 188 (or a variant thereof), an LCDR2 comprising the amino acid sequence set forth in SEQ ID NO: 189 (or a variant thereof), and an LCDR3 comprising the amino acid sequence set forth in SEQ ID NO: 190 (or a variant thereof); (t) a HCVR that comprises: an HCDR1 comprising the amino acid sequence set forth in SEQ ID NO: 193 (or a variant thereof), an HCDR2 comprising the amino acid sequence set forth in SEQ ID NO: 194 (or a variant thereof), and an HCDR3 comprising the amino acid sequence set forth in SEQ ID NO: 195 (or a variant thereof); and a LCVR that comprises: an LCDR1 comprising the amino acid sequence set forth in SEQ ID NO: 198 (or a variant thereof), an LCDR2 comprising the amino acid sequence set forth in SEQ ID NO: 199 (or a variant thereof), and an LCDR3 comprising the amino acid sequence set forth in SEQ ID NO: 200 (or a variant thereof); (u) a HCVR that comprises: an HCDR1 comprising the amino acid sequence set forth in SEQ ID NO: 203 (or a variant thereof), an HCDR2 comprising the amino acid sequence set forth in SEQ ID NO: 204 (or a variant thereof), and an HCDR3 comprising the amino acid sequence set forth in SEQ ID NO: 205 (or a variant thereof); and a LCVR that comprises: an LCDR1 comprising the amino acid sequence set forth in SEQ ID NO: 208 (or a variant thereof), an LCDR2 comprising the amino acid sequence set forth in SEQ ID NO: 209 (or a variant thereof), and an LCDR3 comprising the amino acid sequence set forth in SEQ ID NO: 210 (or a variant thereof); (v) a HCVR that comprises: an HCDR1 comprising the amino acid sequence set forth in SEQ ID NO: 213 (or a variant thereof), an HCDR2 comprising the amino acid sequence set forth in SEQ ID NO: 214 (or a variant thereof), and an HCDR3 comprising the amino acid sequence set forth in SEQ ID NO: 215 (or a variant thereof); and a LCVR that comprises: an LCDR1 comprising the amino acid sequence set forth in SEQ ID NO: 218 (or a variant thereof), an LCDR2 comprising the amino acid sequence set forth in SEQ ID NO: 219 (or a variant thereof), and an LCDR3 comprising the amino acid sequence set forth in SEQ ID NO: 220 (or a variant thereof); (w) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof); (x) a HCVR that comprises: an HCDR1 comprising the amino acid sequence set forth in SEQ ID NO: 233 (or a variant thereof), an HCDR2 comprising the amino acid sequence set forth in SEQ ID NO: 234 (or a variant thereof), and an HCDR3 comprising the amino acid sequence set forth in SEQ ID NO: 235 (or a variant thereof); and a LCVR that comprises: an LCDR1 comprising the amino acid sequence set forth in SEQ ID NO: 238 (or a variant thereof), an LCDR2 comprising the amino acid sequence set forth in SEQ ID NO: 239 (or a variant thereof), and an LCDR3 comprising the amino acid sequence set forth in SEQ ID NO: 240 (or a variant thereof); (y) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof); (z) a HCVR that comprises: an HCDR1 comprising the amino acid sequence set forth in SEQ ID NO: 253 (or a variant thereof), an HCDR2 comprising the amino acid sequence set forth in SEQ ID NO: 254 (or a variant thereof), and an HCDR3 comprising the amino acid sequence set forth in SEQ ID NO: 255 (or a variant thereof); and a LCVR that comprises: an LCDR1 comprising the amino acid sequence set forth in SEQ ID NO: 258 (or a variant thereof), an LCDR2 comprising the amino acid sequence set forth in SEQ ID NO: 259 (or a variant thereof), and an LCDR3 comprising the amino acid sequence set forth in SEQ ID NO: 260 (or a variant thereof); (aa) a HCVR that comprises: an HCDR1 comprising the amino acid sequence set forth in SEQ ID NO: 263 (or a variant thereof), an HCDR2 comprising the amino acid sequence set forth in SEQ ID NO: 264 (or a variant thereof), and an HCDR3 comprising the amino acid sequence set forth in SEQ ID NO: 265 (or a variant thereof); and a LCVR that comprises: an LCDR1 comprising the amino acid sequence set forth in SEQ ID NO: 268 (or a variant thereof), an LCDR2 comprising the amino acid sequence set forth in SEQ ID NO: 269 (or a variant thereof), and an LCDR3 comprising the amino acid sequence set forth in SEQ ID NO: 270 (or a variant thereof); (ab) a HCVR that comprises: an HCDR1 comprising the amino acid sequence set forth in SEQ ID NO: 273 (or a variant thereof), an HCDR2 comprising the amino acid sequence set forth in SEQ ID NO: 274 (or a variant thereof), and an HCDR3 comprising the amino acid sequence set forth in SEQ ID NO: 275 (or a variant thereof); and a LCVR that comprises: an LCDR1 comprising the amino acid sequence set forth in SEQ ID NO: 278 (or a variant thereof), an LCDR2 comprising the amino acid sequence set forth in SEQ ID NO: 279 (or a variant thereof), and an LCDR3 comprising the amino acid sequence set forth in SEQ ID NO: 280 (or a variant thereof); (ac) a HCVR that comprises: an HCDR1 comprising the amino acid sequence set forth in SEQ ID NO: 283 (or a variant thereof), an HCDR2 comprising the amino acid sequence set forth in SEQ ID NO: 284 (or a variant thereof), and an HCDR3 comprising the amino acid sequence set forth in SEQ ID NO: 285 (or a variant thereof); and a LCVR that comprises: an LCDR1 comprising the amino acid sequence set forth in SEQ ID NO: 288 (or a variant thereof), an LCDR2 comprising the amino acid sequence set forth in SEQ ID NO: 289 (or a variant thereof), and an LCDR3 comprising the amino acid sequence set forth in SEQ ID NO: 290 (or a variant thereof); (ad) a HCVR that comprises: an HCDR1 comprising the amino acid sequence set forth in SEQ ID NO: 293 (or a variant thereof), an HCDR2 comprising the amino acid sequence set forth in SEQ ID NO: 294 (or a variant thereof), and an HCDR3 comprising the amino acid sequence set forth in SEQ ID NO: 295 (or a variant thereof); and a LCVR that comprises: an LCDR1 comprising the amino acid sequence set forth in SEQ ID NO: 298 (or a variant thereof), an LCDR2 comprising the amino acid sequence set forth in SEQ ID NO: 299 (or a variant thereof), and an LCDR3 comprising the amino acid sequence set forth in SEQ ID NO: 300 (or a variant thereof); (ae) a HCVR that comprises: an HCDR1 comprising the amino acid sequence set forth in SEQ ID NO: 303 (or a variant thereof), an HCDR2 comprising the amino acid sequence set forth in SEQ ID NO: 304 (or a variant thereof), and an HCDR3 comprising the amino acid sequence set forth in SEQ ID NO: 305 (or a variant thereof); and a LCVR that comprises: an LCDR1 comprising the amino acid sequence set forth in SEQ ID NO: 308 (or a variant thereof), an LCDR2 comprising the amino acid sequence set forth in SEQ ID NO: 309 (or a variant thereof), and an LCDR3 comprising the amino acid sequence set forth in SEQ ID NO: 310 (or a variant thereof); and/or (af) a HCVR that comprises: an HCDR1 comprising the amino acid sequence set forth in SEQ ID NO: 313 (or a variant thereof), an HCDR2 comprising the amino acid sequence set forth in SEQ ID NO: 314 (or a variant thereof), and an HCDR3 comprising the amino acid sequence set forth in SEQ ID NO: 315 (or a variant thereof); and a LCVR that comprises: an LCDR1 comprising the amino acid sequence set forth in SEQ ID NO: 318 (or a variant thereof), an LCDR2 comprising the amino acid sequence set forth in SEQ ID NO: 319 (or a variant thereof), and an LCDR3 comprising the amino acid sequence set forth in SEQ ID NO: 320 (or a variant thereof).

In some embodiments, the antigen-binding protein of the protein-drug conjugate comprises: (i) a HCVR that comprises the amino acid sequence set forth in SEQ ID NO: 2 (or a variant thereof); and a LCVR that comprises the amino acid sequence set forth in SEQ ID NO: 7 (or a variant thereof); (ii) a HCVR that comprises the amino acid sequence set forth in SEQ ID NO: 12 (or a variant thereof); and a LCVR that comprises the amino acid sequence set forth in SEQ ID NO: 17 (or a variant thereof); (iii) a HCVR that comprises the amino acid sequence set forth in SEQ ID NO: 22 (or a variant thereof); and a LCVR that comprises the amino acid sequence set forth in SEQ ID NO: 27 (or a variant thereof); (iv) a HCVR that comprises the amino acid sequence set forth in SEQ ID NO: 32 (or a variant thereof); and a LCVR that comprises the amino acid sequence set forth in SEQ ID NO: 37 (or a variant thereof); (v) a HCVR that comprises the amino acid sequence set forth in SEQ ID NO: 42 (or a variant thereof); and a LCVR that comprises the amino acid sequence set forth in SEQ ID NO: 47 (or a variant thereof); (vi) a HCVR that comprises the amino acid sequence set forth in SEQ ID NO: 52 (or a variant thereof); and a LCVR that comprises the amino acid sequence set forth in SEQ ID NO: 57 (or a variant thereof); (vii) a HCVR that comprises the amino acid sequence set forth in SEQ ID NO: 62 (or a variant thereof); and a LCVR that comprises the amino acid sequence set forth in SEQ ID NO: 67 (or a variant thereof); (viii) a HCVR that comprises the amino acid sequence set forth in SEQ ID NO: 72 (or a variant thereof); and a LCVR that comprises the amino acid sequence set forth in SEQ ID NO: 77 (or a variant thereof); (ix) a HCVR that comprises the amino acid sequence set forth in SEQ ID NO: 82 (or a variant thereof); and a LCVR that comprises the amino acid sequence set forth in SEQ ID NO: 87 (or a variant thereof); (x) a HCVR that comprises the amino acid sequence set forth in SEQ ID NO: 92 (or a variant thereof); and a LCVR that comprises the amino acid sequence set forth in SEQ ID NO: 97 (or a variant thereof); (xi) a HCVR that comprises the amino acid sequence set forth in SEQ ID NO: 102 (or a variant thereof); and a LCVR that comprises the amino acid sequence set forth in SEQ ID NO: 107 (or a variant thereof); (xii) a HCVR that comprises the amino acid sequence set forth in SEQ ID NO: 112 (or a variant thereof); and a LCVR that comprises the amino acid sequence set forth in SEQ ID NO: 117 (or a variant thereof); (xiii) a HCVR that comprises the amino acid sequence set forth in SEQ ID NO: 122 (or a variant thereof); and a LCVR that comprises the amino acid sequence set forth in SEQ ID NO: 127 (or a variant thereof); (xiv) a HCVR that comprises the amino acid sequence set forth in SEQ ID NO: 132 (or a variant thereof); and a LCVR that comprises the amino acid sequence set forth in SEQ ID NO: 137 (or a variant thereof); (xv) a HCVR that comprises the amino acid sequence set forth in SEQ ID NO: 142 (or a variant thereof); and a LCVR that comprises the amino acid sequence set forth in SEQ ID NO: 147 (or a variant thereof); (xvi) a HCVR that comprises the amino acid sequence set forth in SEQ ID NO: 152 (or a variant thereof); and a LCVR that comprises the amino acid sequence set forth in SEQ ID NO: 157 (or a variant thereof); (xvii) a HCVR that comprises the amino acid sequence set forth in SEQ ID NO: 162 (or a variant thereof); and a LCVR that comprises the amino acid sequence set forth in SEQ ID NO: 167 (or a variant thereof); (xviii) a HCVR that comprises the amino acid sequence set forth in SEQ ID NO: 172 (or a variant thereof); and a LCVR that comprises the amino acid sequence set forth in SEQ ID NO: 177 (or a variant thereof); (xix) a HCVR that comprises the amino acid sequence set forth in SEQ ID NO: 182 (or a variant thereof); and a LCVR that comprises the amino acid sequence set forth in SEQ ID NO: 187 (or a variant thereof); (xx) a HCVR that comprises the amino acid sequence set forth in SEQ ID NO: 192 (or a variant thereof); and a LCVR that comprises the amino acid sequence set forth in SEQ ID NO: 197 (or a variant thereof); (xxi) a HCVR that comprises the amino acid sequence set forth in SEQ ID NO: 202 (or a variant thereof); and a LCVR that comprises the amino acid sequence set forth in SEQ ID NO: 207 (or a variant thereof); (xxii) a HCVR that comprises the amino acid sequence set forth in SEQ ID NO: 212 (or a variant thereof); and a LCVR that comprises the amino acid sequence set forth in SEQ ID NO: 217 (or a variant thereof); (xxiii) a HCVR that comprises the amino acid sequence set forth in SEQ ID NO: 222 (or a variant thereof); and a LCVR that comprises the amino acid sequence set forth in SEQ ID NO: 227 (or a variant thereof); (xxiv) a HCVR that comprises the amino acid sequence set forth in SEQ ID NO: 232 (or a variant thereof); and a LCVR that comprises the amino acid sequence set forth in SEQ ID NO: 237 (or a variant thereof); (xxv) a HCVR that comprises the amino acid sequence set forth in SEQ ID NO: 242 (or a variant thereof); and a LCVR that comprises the amino acid sequence set forth in SEQ ID NO: 247 (or a variant thereof); (xxvi) a HCVR that comprises the amino acid sequence set forth in SEQ ID NO: 252 (or a variant thereof); and a LCVR that comprises the amino acid sequence set forth in SEQ ID NO: 257 (or a variant thereof); (xxvii) a HCVR that comprises the amino acid sequence set forth in SEQ ID NO: 262 (or a variant thereof); and a LCVR that comprises the amino acid sequence set forth in SEQ ID NO: 267 (or a variant thereof); (xxviii) a HCVR that comprises the amino acid sequence set forth in SEQ ID NO: 272 (or a variant thereof); and a LCVR that comprises the amino acid sequence set forth in SEQ ID NO: 277 (or a variant thereof); (xxix) a HCVR that comprises the amino acid sequence set forth in SEQ ID NO: 282 (or a variant thereof); and a LCVR that comprises the amino acid sequence set forth in SEQ ID NO: 287 (or a variant thereof); (xxx) a HCVR that comprises the amino acid sequence set forth in SEQ ID NO: 292 (or a variant thereof); and a LCVR that comprises the amino acid sequence set forth in SEQ ID NO: 297 (or a variant thereof); (xxxi) a HCVR that comprises the amino acid sequence set forth in SEQ ID NO: 302 (or a variant thereof); and a LCVR that comprises the amino acid sequence set forth in SEQ ID NO: 307 or 527 (or a variant thereof); and/or (xxxii) a HCVR that comprises the amino acid sequence set forth in SEQ ID NO: 312 (or a variant thereof); and a LCVR that comprises the amino acid sequence set forth in SEQ ID NO: 317 (or a variant thereof).

In some embodiments, the antigen-binding protein of protein-drug conjugate comprises: i. a heavy chain region that comprises the amino acid sequence set forth in SEQ ID NO: 329 (or a variant thereof); and a light chain that comprises the amino acid sequence set forth in SEQ ID NO: 328 (or a variant thereof); ii. a heavy chain region that comprises the amino acid sequence set forth in SEQ ID NO: 331 (or a variant thereof); and a light chain that comprises the amino acid sequence set forth in SEQ ID NO: 330 (or a variant thereof); iii. a heavy chain region that comprises the amino acid sequence set forth in SEQ ID NO: 333 (or a variant thereof); and a light chain that comprises the amino acid sequence set forth in SEQ ID NO: 332 (or a variant thereof); iv. a heavy chain region that comprises the amino acid sequence set forth in SEQ ID NO: 335 (or a variant thereof); and a light chain that comprises the amino acid sequence set forth in SEQ ID NO: 334 (or a variant thereof); v. a heavy chain region that comprises the amino acid sequence set forth in SEQ ID NO: 337 (or a variant thereof); and a light chain that comprises the amino acid sequence set forth in SEQ ID NO: 336 (or a variant thereof); vi. a heavy chain region that comprises the amino acid sequence set forth in SEQ ID NO: 339 (or a variant thereof); and a light chain that comprises the amino acid sequence set forth in SEQ ID NO: 338 (or a variant thereof); vii. a heavy chain region that comprises the amino acid sequence set forth in SEQ ID NO: 341 (or a variant thereof); and a light chain that comprises the amino acid sequence set forth in SEQ ID NO: 340 (or a variant thereof); viii. a heavy chain region that comprises the amino acid sequence set forth in SEQ ID NO: 343 (or a variant thereof); and a light chain that comprises the amino acid sequence set forth in SEQ ID NO: 342 (or a variant thereof); ix. a heavy chain region that comprises the amino acid sequence set forth in SEQ ID NO: 345 (or a variant thereof); and a light chain that comprises the amino acid sequence set forth in SEQ ID NO: 344 (or a variant thereof); x. a heavy chain region that comprises the amino acid sequence set forth in SEQ ID NO: 347 (or a variant thereof); and a light chain that comprises the amino acid sequence set forth in SEQ ID NO: 346 (or a variant thereof); xi. a heavy chain region that comprises the amino acid sequence set forth in SEQ ID NO: 349 (or a variant thereof); and a light chain that comprises the amino acid sequence set forth in SEQ ID NO: 348 (or a variant thereof); xii. a heavy chain region that comprises the amino acid sequence set forth in SEQ ID NO: 351 (or a variant thereof); and a light chain that comprises the amino acid sequence set forth in SEQ ID NO: 350 (or a variant thereof); xiii. a heavy chain region that comprises the amino acid sequence set forth in SEQ ID NO: 353 (or a variant thereof); and a light chain that comprises the amino acid sequence set forth in SEQ ID NO: 352 (or a variant thereof); xiv. a heavy chain region that comprises the amino acid sequence set forth in SEQ ID NO: 355 (or a variant thereof); and a light chain that comprises the amino acid sequence set forth in SEQ ID NO: 354 (or a variant thereof); xv. a heavy chain region that comprises the amino acid sequence set forth in SEQ ID NO: 357 (or a variant thereof); and a light chain that comprises the amino acid sequence set forth in SEQ ID NO: 356 (or a variant thereof); xvi. a heavy chain region that comprises the amino acid sequence set forth in SEQ ID NO: 359 (or a variant thereof); and a light chain that comprises the amino acid sequence set forth in SEQ ID NO: 358 (or a variant thereof); xvii. a heavy chain region that comprises the amino acid sequence set forth in SEQ ID NO: 361 (or a variant thereof); and a light chain that comprises the amino acid sequence set forth in SEQ ID NO: 360 (or a variant thereof); xviii. a heavy chain region that comprises the amino acid sequence set forth in SEQ ID NO: 363 (or a variant thereof); and a light chain that comprises the amino acid sequence set forth in SEQ ID NO: 362 (or a variant thereof); xix. a heavy chain region that comprises the amino acid sequence set forth in SEQ ID NO: 365 (or a variant thereof); and a light chain that comprises the amino acid sequence set forth in SEQ ID NO: 364 (or a variant thereof); xx. a heavy chain region that comprises the amino acid sequence set forth in SEQ ID NO: 367 (or a variant thereof); and a light chain that comprises the amino acid sequence set forth in SEQ ID NO: 366 (or a variant thereof); xxi. a heavy chain region that comprises the amino acid sequence set forth in SEQ ID NO: 369 (or a variant thereof); and a light chain that comprises the amino acid sequence set forth in SEQ ID NO: 368 (or a variant thereof); xxii. a heavy chain region that comprises the amino acid sequence set forth in SEQ ID NO: 371 (or a variant thereof); and a light chain that comprises the amino acid sequence set forth in SEQ ID NO: 370 (or a variant thereof); xxiii. a heavy chain region that comprises the amino acid sequence set forth in SEQ ID NO: 373 (or a variant thereof); and a light chain that comprises the amino acid sequence set forth in SEQ ID NO: 372 (or a variant thereof); xxiv. a heavy chain region that comprises the amino acid sequence set forth in SEQ ID NO: 375 (or a variant thereof); and a light chain that comprises the amino acid sequence set forth in SEQ ID NO: 374 (or a variant thereof); xxv. a heavy chain region that comprises the amino acid sequence set forth in SEQ ID NO: 377 (or a variant thereof); and a light chain that comprises the amino acid sequence set forth in SEQ ID NO: 376 (or a variant thereof); xxvi. a heavy chain region that comprises the amino acid sequence set forth in SEQ ID NO: 379 (or a variant thereof); and a light chain that comprises the amino acid sequence set forth in SEQ ID NO: 378 (or a variant thereof); xxvii. a heavy chain region that comprises the amino acid sequence set forth in SEQ ID NO: 381 (or a variant thereof); and a light chain that comprises the amino acid sequence set forth in SEQ ID NO: 380 (or a variant thereof); xxviii. a heavy chain region that comprises the amino acid sequence set forth in SEQ ID NO: 383 (or a variant thereof); and a light chain that comprises the amino acid sequence set forth in SEQ ID NO: 382 (or a variant thereof); xxix. a heavy chain region that comprises the amino acid sequence set forth in SEQ ID NO: 385 (or a variant thereof); and a light chain that comprises the amino acid sequence set forth in SEQ ID NO: 384 (or a variant thereof); xxx. a heavy chain region that comprises the amino acid sequence set forth in SEQ ID NO: 387 (or a variant thereof); and a light chain that comprises the amino acid sequence set forth in SEQ ID NO: 386 (or a variant thereof); xxxi. a heavy chain region that comprises the amino acid sequence set forth in SEQ ID NO: 389 (or a variant thereof); and a light chain that comprises the amino acid sequence set forth in SEQ ID NO: 388 (or a variant thereof); or xxxii. a heavy chain region that comprises the amino acid sequence set forth in SEQ ID NO: 391 (or a variant thereof); and a light chain that comprises the amino acid sequence set forth in SEQ ID NO: 390 (or a variant thereof).

In some embodiments, the antigen-binding protein of the protein-drug conjugate comprises: i. a heavy chain that comprises the amino acid sequence set forth in SEQ ID NO: 543 (or a variant thereof); and a light chain that comprises the amino acid sequence set forth in SEQ ID NO: 328 (or a variant thereof); ii. a heavy chain that comprises the amino acid sequence set forth in SEQ ID NO: 544 (or a variant thereof); and a light chain that comprises the amino acid sequence set forth in SEQ ID NO: 330 (or a variant thereof); iii. a heavy chain that comprises the amino acid sequence set forth in SEQ ID NO: 545 (or a variant thereof); and a light chain that comprises the amino acid sequence set forth in SEQ ID NO: 332 (or a variant thereof); iv. a heavy chain that comprises the amino acid sequence set forth in SEQ ID NO: 546 (or a variant thereof); and a light chain that comprises the amino acid sequence set forth in SEQ ID NO: 334 (or a variant thereof); v. a heavy chain that comprises the amino acid sequence set forth in SEQ ID NO: 547 (or a variant thereof); and a light chain that comprises the amino acid sequence set forth in SEQ ID NO: 336 (or a variant thereof); vi. a heavy chain that comprises the amino acid sequence set forth in SEQ ID NO: 548 (or a variant thereof); and a light chain that comprises the amino acid sequence set forth in SEQ ID NO: 338 (or a variant thereof); vii. a heavy chain that comprises the amino acid sequence set forth in SEQ ID NO: 549 (or a variant thereof); and a light chain that comprises the amino acid sequence set forth in SEQ ID NO: 340 (or a variant thereof); viii. a heavy chain that comprises the amino acid sequence set forth in SEQ ID NO: 550 (or a variant thereof); and a light chain that comprises the amino acid sequence set forth in SEQ ID NO: 342 (or a variant thereof); ix. a heavy chain that comprises the amino acid sequence set forth in SEQ ID NO: 551 (or a variant thereof); and a light chain that comprises the amino acid sequence set forth in SEQ ID NO: 344 (or a variant thereof); x. a heavy chain that comprises the amino acid sequence set forth in SEQ ID NO: 552 (or a variant thereof); and a light chain that comprises the amino acid sequence set forth in SEQ ID NO: 346 (or a variant thereof); xi. a heavy chain that comprises the amino acid sequence set forth in SEQ ID NO: 553 (or a variant thereof); and a light chain that comprises the amino acid sequence set forth in SEQ ID NO: 348 (or a variant thereof); xii. a heavy chain that comprises the amino acid sequence set forth in SEQ ID NO: 554 (or a variant thereof); and a light chain that comprises the amino acid sequence set forth in SEQ ID NO: 350 (or a variant thereof); xiii. a heavy chain that comprises the amino acid sequence set forth in SEQ ID NO: 555 (or a variant thereof); and a light chain that comprises the amino acid sequence set forth in SEQ ID NO: 352 (or a variant thereof); xiv. a heavy chain that comprises the amino acid sequence set forth in SEQ ID NO: 556 (or a variant thereof); and a light chain that comprises the amino acid sequence set forth in SEQ ID NO: 354 (or a variant thereof); xv. a heavy chain that comprises the amino acid sequence set forth in SEQ ID NO: 557 (or a variant thereof); and a light chain that comprises the amino acid sequence set forth in SEQ ID NO: 356 (or a variant thereof); xvi. a heavy chain that comprises the amino acid sequence set forth in SEQ ID NO: 558 (or a variant thereof); and a light chain that comprises the amino acid sequence set forth in SEQ ID NO: 358 (or a variant thereof); xvii. a heavy chain that comprises the amino acid sequence set forth in SEQ ID NO: 559 (or a variant thereof); and a light chain that comprises the amino acid sequence set forth in SEQ ID NO: 360 (or a variant thereof); xviii. a heavy chain that comprises the amino acid sequence set forth in SEQ ID NO: 560 (or a variant thereof); and a light chain that comprises the amino acid sequence set forth in SEQ ID NO: 362 (or a variant thereof); xix. a heavy chain that comprises the amino acid sequence set forth in SEQ ID NO: 561 (or a variant thereof); and a light chain that comprises the amino acid sequence set forth in SEQ ID NO: 364 (or a variant thereof); xx. a heavy chain that comprises the amino acid sequence set forth in SEQ ID NO: 562 (or a variant thereof); and a light chain that comprises the amino acid sequence set forth in SEQ ID NO: 366 (or a variant thereof); xxi. a heavy chain that comprises the amino acid sequence set forth in SEQ ID NO: 563 (or a variant thereof); and a light chain that comprises the amino acid sequence set forth in SEQ ID NO: 368 (or a variant thereof); xxii. a heavy chain that comprises the amino acid sequence set forth in SEQ ID NO: 564 (or a variant thereof); and a light chain that comprises the amino acid sequence set forth in SEQ ID NO: 370 (or a variant thereof); xxiii. a heavy chain that comprises the amino acid sequence set forth in SEQ ID NO: 565 (or a variant thereof); and a light chain that comprises the amino acid sequence set forth in SEQ ID NO: 372 (or a variant thereof); xxiv. a heavy chain that comprises the amino acid sequence set forth in SEQ ID NO: 566 (or a variant thereof); and a light chain that comprises the amino acid sequence set forth in SEQ ID NO: 374 (or a variant thereof); xxv. a heavy chain that comprises the amino acid sequence set forth in SEQ ID NO: 567 (or a variant thereof); and a light chain that comprises the amino acid sequence set forth in SEQ ID NO: 376 (or a variant thereof); xxvi. a heavy chain that comprises the amino acid sequence set forth in SEQ ID NO: 568 (or a variant thereof); and a light chain that comprises the amino acid sequence set forth in SEQ ID NO: 378 (or a variant thereof); xxvii. a heavy chain that comprises the amino acid sequence set forth in SEQ ID NO: 569 (or a variant thereof); and a light chain that comprises the amino acid sequence set forth in SEQ ID NO: 380 (or a variant thereof); xxviii. a heavy chain that comprises the amino acid sequence set forth in SEQ ID NO: 570 (or a variant thereof); and a light chain that comprises the amino acid sequence set forth in SEQ ID NO: 382 (or a variant thereof); xxix. a heavy chain that comprises the amino acid sequence set forth in SEQ ID NO: 571 (or a variant thereof); and a light chain that comprises the amino acid sequence set forth in SEQ ID NO: 384 (or a variant thereof); xxx. a heavy chain that comprises the amino acid sequence set forth in SEQ ID NO: 572 (or a variant thereof); and a light chain that comprises the amino acid sequence set forth in SEQ ID NO: 386 (or a variant thereof); xxxi. a heavy chain that comprises the amino acid sequence set forth in SEQ ID NO: 573 (or a variant thereof); and a light chain that comprises the amino acid sequence set forth in SEQ ID NO: 388 (or a variant thereof); or xxxii. a heavy chain that comprises the amino acid sequence set forth in SEQ ID NO: 574 (or a variant thereof); and a light chain that comprises the amino acid sequence set forth in SEQ ID NO: 390 (or a variant thereof).

In some embodiments, the antigen-binding protein of the protein-drug conjugate comprises: (1) a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof); (2) a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof); (3) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof); (4) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof); (5) a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof); or (6) a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof).

In some embodiments, the antigen-binding protein of the protein-drug conjugate comprises: (a) a HCVR that comprises: an HCDR1 comprising the amino acid sequence set forth in SEQ ID NO: 133 (or a variant thereof), an HCDR2 comprising the amino acid sequence set forth in SEQ ID NO: 134 (or a variant thereof), and an HCDR3 comprising the amino acid sequence set forth in SEQ ID NO: 135 (or a variant thereof); and a LCVR that comprises: an LCDR1 comprising the amino acid sequence set forth in SEQ ID NO: 138 (or a variant thereof), an LCDR2 comprising the amino acid sequence set forth in SEQ ID NO: 139 (or a variant thereof), and an LCDR3 comprising the amino acid sequence set forth in SEQ ID NO: 140 (or a variant thereof); (b) a HCVR that comprises: an HCDR1 comprising the amino acid sequence set forth in SEQ ID NO: 173 (or a variant thereof), an HCDR2 comprising the amino acid sequence set forth in SEQ ID NO: 174 (or a variant thereof), and an HCDR3 comprising the amino acid sequence set forth in SEQ ID NO: 175 (or a variant thereof); and a LCVR that comprises: an LCDR1 comprising the amino acid sequence set forth in SEQ ID NO: 178 (or a variant thereof), an LCDR2 comprising the amino acid sequence set forth in SEQ ID NO: 179 (or a variant thereof), and an LCDR3 comprising the amino acid sequence set forth in SEQ ID NO: 180 (or a variant thereof); (c) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof); (d) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof); (e) a HCVR that comprises: an HCDR1 comprising the amino acid sequence set forth in SEQ ID NO: 263 (or a variant thereof), an HCDR2 comprising the amino acid sequence set forth in SEQ ID NO: 264 (or a variant thereof), and an HCDR3 comprising the amino acid sequence set forth in SEQ ID NO: 265 (or a variant thereof); and a LCVR that comprises: an LCDR1 comprising the amino acid sequence set forth in SEQ ID NO: 268 (or a variant thereof), an LCDR2 comprising the amino acid sequence set forth in SEQ ID NO: 269 (or a variant thereof), and an LCDR3 comprising the amino acid sequence set forth in SEQ ID NO: 270 (or a variant thereof); or (f) a HCVR that comprises: an HCDR1 comprising the amino acid sequence set forth in SEQ ID NO: 273 (or a variant thereof), an HCDR2 comprising the amino acid sequence set forth in SEQ ID NO: 274 (or a variant thereof), and an HCDR3 comprising the amino acid sequence set forth in SEQ ID NO: 275 (or a variant thereof); and a LCVR that comprises: an LCDR1 comprising the amino acid sequence set forth in SEQ ID NO: 278 (or a variant thereof), an LCDR2 comprising the amino acid sequence set forth in SEQ ID NO: 279 (or a variant thereof), and an LCDR3 comprising the amino acid sequence set forth in SEQ ID NO: 280 (or a variant thereof).

In some embodiments, the protein-drug conjugate comprises: (i) a HCVR that comprises the amino acid sequence set forth in SEQ ID NO: 132 (or a variant thereof); and a LCVR that comprises the amino acid sequence set forth in SEQ ID NO: 137 (or a variant thereof); (ii) a HCVR that comprises the amino acid sequence set forth in SEQ ID NO: 172 (or a variant thereof); and a LCVR that comprises the amino acid sequence set forth in SEQ ID NO: 177 (or a variant thereof); (iii) a HCVR that comprises the amino acid sequence set forth in SEQ ID NO: 222 (or a variant thereof); and a LCVR that comprises the amino acid sequence set forth in SEQ ID NO: 227 (or a variant thereof); (iv) a HCVR that comprises the amino acid sequence set forth in SEQ ID NO: 242 (or a variant thereof); and a LCVR that comprises the amino acid sequence set forth in SEQ ID NO: 247 (or a variant thereof); (v) a HCVR that comprises the amino acid sequence set forth in SEQ ID NO: 262 (or a variant thereof); and a LCVR that comprises the amino acid sequence set forth in SEQ ID NO: 267 (or a variant thereof); or (vi) a HCVR that comprises the amino acid sequence set forth in SEQ ID NO: 272 (or a variant thereof); and a LCVR that comprises the amino acid sequence set forth in SEQ ID NO: 277 (or a variant thereof).

In some embodiments, the antigen-binding protein of the protein-drug conjugate comprises: (A) a heavy chain region that comprises the amino acid sequence set forth in SEQ ID NO: 355 (or a variant thereof); and a light chain that comprises the amino acid sequence set forth in SEQ ID NO: 354 (or a variant thereof); (B) a heavy chain region that comprises the amino acid sequence set forth in SEQ ID NO: 363 (or a variant thereof); and a light chain that comprises the amino acid sequence set forth in SEQ ID NO: 362 (or a variant thereof); (C) a heavy chain region that comprises the amino acid sequence set forth in SEQ ID NO: 373 (or a variant thereof); and a light chain that comprises the amino acid sequence set forth in SEQ ID NO: 372 (or a variant thereof); (D) a heavy chain region that comprises the amino acid sequence set forth in SEQ ID NO: 377 (or a variant thereof); and a light chain that comprises the amino acid sequence set forth in SEQ ID NO: 376 (or a variant thereof); (E) a heavy chain region that comprises the amino acid sequence set forth in SEQ ID NO: 381 (or a variant thereof); and a light chain that comprises the amino acid sequence set forth in SEQ ID NO: 380 (or a variant thereof); or (F) a heavy chain region that comprises the amino acid sequence set forth in SEQ ID NO: 383 (or a variant thereof); and a light chain that comprises the amino acid sequence set forth in SEQ ID NO: 382 (or a variant thereof).

In some embodiments, the antigen-binding protein of the protein-drug conjugate comprises: (I) a heavy chain that comprises the amino acid sequence set forth in SEQ ID NO: 556 (or a variant thereof); and a light chain that comprises the amino acid sequence set forth in SEQ ID NO: 354 (or a variant thereof); (II) a heavy chain that comprises the amino acid sequence set forth in SEQ ID NO: 560 (or a variant thereof); and a light chain that comprises the amino acid sequence set forth in SEQ ID NO: 362 (or a variant thereof); (III) a heavy chain that comprises the amino acid sequence set forth in SEQ ID NO: 565 (or a variant thereof); and a light chain that comprises the amino acid sequence set forth in SEQ ID NO: 372 (or a variant thereof); (IV) a heavy chain that comprises the amino acid sequence set forth in SEQ ID NO: 367 (or a variant thereof); and a light chain that comprises the amino acid sequence set forth in SEQ ID NO: 376 (or a variant thereof); (V) a heavy chain that comprises the amino acid sequence set forth in SEQ ID NO: 569 (or a variant thereof); and a light chain that comprises the amino acid sequence set forth in SEQ ID NO: 380 (or a variant thereof); or (VI) a heavy chain that comprises the amino acid sequence set forth in SEQ ID NO: 570 (or a variant thereof); and a light chain that comprises the amino acid sequence set forth in SEQ ID NO: 382 (or a variant thereof).

In some embodiments, the antigen-binding protein binds to the same epitope on human transferrin receptor as an antibody comprising an HCVR/LCVR amino acid sequence pair as set forth in Table 1-1.

In some embodiments, the antigen-binding protein competes for binding to human transferrin receptor with an antibody comprising an HCVR/LCVR amino acid sequence pair as set forth in Table 1-1.

In another aspect, provided herein is a protein-drug conjugate comprising an antigen-binding protein that binds specifically to human transferrin receptor (hTfR), wherein the antigen-binding protein is conjugated to a molecular cargo and comprises an antibody or antigen-binding fragment thereof, wherein the antibody or antigen-binding fragment thereof binds to one or more epitopes of hTfR selected from:
a. an epitope comprising the sequence LLNE (SEQ ID NO: 529) and/or an epitope comprising the sequence TYKEL (SEQ ID NO: 509);
b. an epitope comprising the sequence DSTDFTGT (SEQ ID NO: 530) and/or an epitope comprising the sequence VKHPVTGQF (SEQ ID NO: 531) and/or an epitope comprising the sequence IERIPEL (SEQ ID NO: 532);
c. an epitope comprising the sequence LNENSYVPREAGSQKDEN (SEQ ID NO: 533);
d. an epitope comprising the sequence FEDL (SEQ ID NO: 521);
e. an epitope comprising the sequence IVDKNGRL (SEQ ID NO: 534);
f. an epitope comprising the sequence IVDKNGRLVY (SEQ ID NO: 535);
g. an epitope comprising the sequence DQTKF (SEQ ID NO: 536);
h. an epitope comprising the sequence LVENPGGY (SEQ ID NO: 537) and/or an epitope comprising the sequence PIVNAELSF (SEQ ID NO: 538) and/or an epitope comprising the sequence PYLGTTMDT (SEQ ID NO: 539);
i. an epitope comprising the sequence LLNENSYVPREAGSQKDENLAL (SEQ ID NO: 507) and/or an epitope comprising the sequence IYMDQTKFPIVNAEL (SEQ ID NO: 508) and/or an epitope comprising the sequence TYKEL (SEQ ID NO: 509);
j. an epitope comprising the sequence KRKLSEKLDSTDFTGTIKL (SEQ ID NO: 510) and/or an epitope comprising the sequence YTLIEKTMQNVKHPVTGQFL (SEQ ID NO: 511) and/or an epitope comprising the sequence LIERIPELNKVARAAAE (SEQ ID NO: 512);
k. an epitope comprising the sequence LNENSYVPREAGSQKDENL (SEQ ID NO: 513);
l. an epitope comprising the sequence GTKKDFEDL (SEQ ID NO: 514);
m. an epitope comprising the sequence SVIIVDKNGRLVYLVENPGGYVAYSK (SEQ ID NO: 515);
n. an epitope comprising the sequence LLNENSYVPREAGSQKDEN (SEQ ID NO: 516) and/or an epitope comprising the sequence DQTKFPIVNAEL (SEQ ID NO: 517) and/or an epitope comprising the sequence TYKELIERIPELNK (SEQ ID NO: 518);
o. an epitope comprising the sequence LLNENSYVPREAGSQKDEN (SEQ ID NO: 516) and/or an epitope comprising the sequence TYKELIERIPELNK (SEQ ID NO: 518);
p. an epitope comprising the sequence SVIIVDKNGRLVYLVENPGGYVAY (SEQ ID NO: 519);
q. an epitope comprising the sequence IYMDQTKFPIVNAEL (SEQ ID NO: 508) and/or an epitope comprising the sequence FGNMEGDCPSDWKTDSTCRM (SEQ ID NO: 520);
r. an epitope comprising the sequence LLNENSYVPREAGSQKDENLAL (SEQ ID NO: 507) and/or an epitope comprising the sequence LVENPGGYVAYSKAATVTGKL (SEQ ID NO: 522) and/or an epitope comprising the sequence IYMDQTKFPIVNAELSF (SEQ ID NO: 523) and/or an epitope comprising the sequence ISRAAAEKL (SEQ ID NO: 524) and/or an epitope comprising the sequence VTSESKNVKLTVSNVLKE (SEQ ID NO: 525) and/or an epitope comprising the sequence FCEDTDYPYLGTTMDT (SEQ ID NO: 526);
s. an epitope comprised within or overlapping with the sequence LLNENSYVPREAGSQKDENLAL (SEQ ID NO: 507) and/or an epitope comprised within or overlapping with the sequence IYMDQTKFPIVNAEL (SEQ ID NO: 508) and/or an epitope comprised within or overlapping with the sequence TYKEL (SEQ ID NO: 509);
t. an epitope comprised within or overlapping with the sequence KRKLSEKLDSTDFTGTIKL (SEQ ID NO: 510) and/or an epitope comprised within or overlapping with the sequence YTLIEKTMQNVKHPVTGQFL (SEQ ID NO: 511) and/or an epitope comprised within or overlapping with the sequence LIERIPELNKVARAAAE (SEQ ID NO: 512);
u. an epitope comprised within or overlapping with the sequence LNENSYVPREAGSQKDENL (SEQ ID NO: 513);
v. an epitope comprised within or overlapping with the sequence GTKKDFEDL (SEQ ID NO: 514);
w. an epitope comprised within or overlapping with the sequence SVIIVDKNGRLVYLVENPGGYVAYSK (SEQ ID NO: 515);
x. an epitope comprised within or overlapping with the sequence LLNENSYVPREAGSQKDEN (SEQ ID NO: 516) and/or an epitope comprised within or overlapping with the sequence DQTKFPIVNAEL (SEQ ID NO: 517) and/or an epitope comprised within or overlapping with the sequence TYKELIERIPELNK (SEQ ID NO: 518);
y. an epitope comprised within or overlapping with the sequence LLNENSYVPREAGSQKDEN (SEQ ID NO: 516) and/or an epitope comprised within or overlapping with the sequence TYKELIERIPELNK (SEQ ID NO: 518);
z. an epitope comprised within or overlapping with the sequence SVIIVDKNGRLVYLVENPGGYVAY (SEQ ID NO: 519;
aa. an epitope comprised within or overlapping with the sequence IYMDQTKFPIVNAEL (SEQ ID NO: 508) and/or an epitope comprised within or overlapping with the sequence FGNMEGDCPSDWKTDSTCRM (SEQ ID NO: 520); and
bb. an epitope comprised within or overlapping with the sequence LLNENSYVPREAGSQKDENLAL (SEQ ID NO: 507) and/or an epitope comprised within or overlapping with the sequence LVENPGGYVAYSKAATVTGKL (SEQ ID NO: 522) and/or an epitope comprised within or overlapping with the sequence IYMDQTKFPIVNAELSF (SEQ ID NO: 523) and/or an epitope comprised within or overlapping with the sequence ISRAAAEKL (SEQ ID NO: 524) and/or an epitope comprised within or overlapping with the sequence VTSESKNVKLTVSNVLKE (SEQ ID NO: 525) and/or an epitope comprised within or overlapping with the sequence FCEDTDYPYLGTTMDT (SEQ ID NO: 526).

In some embodiments, the antibody or antigen-binding fragment thereof binds to one or more epitopes of hTfR selected from:
a. an epitope consisting of the sequence LLNE (SEQ ID NO: 529) and/or an epitope consisting of the sequence TYKEL (SEQ ID NO: 509);
b. an epitope consisting of the sequence DSTDFTGT (SEQ ID NO: 530) and/or an epitope consisting of the sequence VKHPVTGQF (SEQ ID NO: 531) and/or an epitope consisting of the sequence IERIPEL (SEQ ID NO: 532);
c. an epitope consisting of the sequence LNENSYVPREAGSQKDEN (SEQ ID NO: 533);
d. an epitope consisting of the sequence FEDL (SEQ ID NO: 521);
e. an epitope consisting of the sequence IVDKNGRL (SEQ ID NO: 534);
f. an epitope consisting of the sequence IVDKNGRLVY (SEQ ID NO: 535);
g. an epitope consisting of the sequence DQTKF (SEQ ID NO: 536);
h. an epitope consisting of the sequence LVENPGGY (SEQ ID NO: 537) and/or an epitope consisting of the sequence PIVNAELSF (SEQ ID NO: 538) and/or an epitope consisting of the sequence PYLGTTMDT (SEQ ID NO: 539);
i. an epitope consisting of the sequence LLNENSYVPREAGSQKDENLAL (SEQ ID NO: 507) and/or an epitope consisting of the sequence IYMDQTKFPIVNAEL (SEQ ID NO: 508) and/or an epitope consisting of the sequence TYKEL (SEQ ID NO: 509);
j. an epitope consisting of the sequence KRKLSEKLDSTDFTGTIKL (SEQ ID NO: 510) and/or an epitope consisting of the sequence YTLIEKTMQNVKHPVTGQFL (SEQ ID NO: 511) and/or an epitope consisting of the sequence LIERIPELNKVARAAAE (SEQ ID NO: 512);
k. an epitope consisting of the sequence LNENSYVPREAGSQKDENL (SEQ ID NO: 513);
l. an epitope consisting of the sequence GTKKDFEDL (SEQ ID NO: 514);
m. an epitope consisting of the sequence SVIIVDKNGRLVYLVENPGGYVAYSK (SEQ ID NO: 515);
n. an epitope consisting of the sequence LLNENSYVPREAGSQKDEN (SEQ ID NO: 516) and/or an epitope consisting of the sequence DQTKFPIVNAEL (SEQ ID NO: 517) and/or an epitope consisting of the sequence TYKELIERIPELNK (SEQ ID NO: 518);
o. an epitope consisting of the sequence LLNENSYVPREAGSQKDEN (SEQ ID NO: 516) and/or an epitope consisting of the sequence TYKELIERIPELNK (SEQ ID NO: 518);
p. an epitope consisting of the sequence SVIIVDKNGRLVYLVENPGGYVAY (SEQ ID NO: 519);
q. an epitope consisting of the sequence IYMDQTKFPIVNAEL (SEQ ID NO: 508) and/or an epitope consisting of the sequence FGNMEGDCPSDWKTDSTCRM (SEQ ID NO: 520); and
r. an epitope consisting of the sequence LLNENSYVPREAGSQKDENLAL (SEQ ID NO: 507) and/or an epitope consisting of the sequence LVENPGGYVAYSKAATVTGKL (SEQ ID NO: 522) and/or an epitope consisting of the sequence IYMDQTKFPIVNAELSF (SEQ ID NO: 523) and/or an epitope consisting of the sequence ISRAAAEKL (SEQ ID NO: 524) and/or an epitope consisting of the sequence VTSESKNVKLTVSNVLKE (SEQ ID NO: 525) and/or an epitope consisting of the sequence FCEDTDYPYLGTTMDT (SEQ ID NO: 526).

In some embodiments, the antigen-binding protein is selected from a humanized antibody or antigen binding fragment thereof, human antibody or antigen binding fragment thereof, murine antibody or antigen binding fragment thereof, chimeric antibody or antigen binding fragment thereof, monovalent Fab′, divalent Fab2, F(ab)′3 fragments, single-chain fragment variable (scFv), bis-scFv, (scFv)2, diabody, minibody, nanobody, triabody, tetrabody, disulfide stabilized Fv protein (dsFv), single-domain antibody (sdAb), Ig NAR, single heavy chain antibody, bispecific antibody or biding fragment thereof, bi-specific T-cell engager (BiTE), trispecific antibody, or chemically modified derivatives thereof.

In some embodiments, the protein-drug conjugate comprises an scFv that comprises a heavy chain variable region (HCVR) and a light chain variable region (LCVR), and a molecular cargo, wherein said molecular cargo is conjugated to the HCVR. In some embodiments, the protein-drug conjugate comprises an scFv that comprises a heavy chain variable region (HCVR) and a light chain variable region (LCVR), and a molecular cargo, wherein said molecular cargo is conjugated to the LCVR. In some embodiments, the scFv and said molecular cargo are conjugated via a linker.

In some embodiments, the molecular cargo is conjugated to: (i) a HCVR of the antigen-binding protein, (ii) a LCVR of the antigen-binding protein, (iii) a heavy chain of the antigen-binding protein, and/or (iv) a light chain of the antigen-binding protein.

In some embodiments, the molecular cargo is conjugated to: (i) one or both HCVRs of the antigen-binding protein, (ii) one or both LCVRs of the antigen-binding protein, (iii) one or both heavy chains of the antigen-binding protein, and/or (iv) one or both light chains of the antigen-binding protein.

In some embodiments, the molecular cargo is conjugated to antigen-binding protein via a glutamine residue and/or a lysine residue.

In some embodiments, the glutamine residue is: (i) introduced to the N-terminus and/or C-terminus of a heavy chain of the antigen-binding protein, (ii) introduced to the N-terminus and/or C-terminus of a light chain of the antigen-binding protein, (iii) naturally present in a CH2 or CH3 domain of the antigen-binding protein, (iv) introduced to the antigen-binding protein by modifying one or more amino acids, and/or (v) Q295 or mutated from N297 to Q297 (N297Q). In one embodiment, the glutamine residue is Q295.

In some embodiments, the antigen-binding protein comprises a glutamine-containing tag, and the molecular cargo is conjugated to the antigen-binding protein via a glutamine residue of the glutamine-containing tag. In some embodiments, the glutamine-containing tag comprises an amino acid sequence selected from the group consisting of LLQGG (SEQ ID NO: 439), LLQG (SEQ ID NO: 440), LSLSQG (SEQ ID NO: 441), gGGLLQGG (SEQ ID NO: 442), gLLQG (SEQ ID NO: 443), LLQ (SEQ ID NO: 444), gSPLAQSHGG (SEQ ID NO: 445), gLLQGGG (SEQ ID NO: 446), gLLQGG (SEQ ID NO: 447), gLLQ (SEQ ID NO: 448), LLQLLQGA (SEQ ID NO: 449), LLQGA (SEQ ID NO: 450), LLQYQGA (SEQ ID NO: 451), LLQGSG (SEQ ID NO: 452), LLQYQG (SEQ ID NO: 453), LLQLLQG (SEQ ID NO: 454), SLLQG (SEQ ID NO: 455), LLQLQ (SEQ ID NO: 456), LLQLLQ (SEQ ID NO: 457), and LLQGR (SEQ ID NO: 458).

In some embodiments, the antigen-binding protein and the molecular cargo are conjugated via a linker. The linker may be a cleavable or non-cleavable linker.

In some embodiments, the protein-drug conjugate comprises a molecular cargo which comprises a polynucleotide molecule, a carrier, or a small molecule.

In some embodiments, the protein-drug conjugate comprises a polynucleotide molecule. In some embodiments, the polynucleotide molecule is an interfering nucleic acid molecule, a guide RNA, a ribozyme, an aptamer, a mixmer, a multimer, or an mRNA. In some embodiments, the interfering nucleic acid is an siRNA, an shRNA, a miRNA, a gapmer, or an antisense oligonucleotide. In some embodiments, the interfering nucleic acid is an siRNA. In some embodiments, the interfering nucleic acid is an antisense oligonucleotide. In some embodiments, the polynucleotide molecule is a guide RNA. In various embodiments, the polynucleotide molecule comprises one or more modified nucleotides.

In some embodiments, the molecular cargo is an siRNA that inhibits the DMPK, CNBP, Dystrophin, DUX4, ApoE, MAPT, APP, HTT, SOD1, C9orf72, SNCA, LRRK2, PRNP, SLC5A1, SLC16A3, HDAC6, MMP27, MFAP5, FAM64A, BAIAP3, MYH7, TPM1, RBM20, KLHL24, MYL2, or TNNT2 gene or a mutant thereof.

In some embodiments, the siRNA comprises a sense strand of 21 nucleotides in length. In some embodiments, the siRNA comprises an antisense strand of 23 nucleotides in length. In some embodiments, the siRNA comprises two phosphorothioate linkages at the first and second internucleoside linkages at the 5′ end of the sense strand. In some embodiments, the siRNA comprises two phosphorothioate linkages at the first and second internucleoside linkages at the 3′ and/or 5′ ends of the antisense strand.

In some embodiments, the molecular cargo comprises a carrier, such as a lipid-based carrier. In some embodiments, the lipid-based carrier is a lipid nanoparticle (LNP), a liposome, a lipidoid, or a lipoplex. In some embodiments, the lipid-based carrier is a lipid nanoparticle (LNP). In some embodiments, the lipid nanoparticle (LNP) further comprises a polynucleotide molecule and/or a polypeptide molecule.

In some embodiments, the lipid nanoparticle (LNP) comprises one or more components of a gene editing system. In some embodiments, the lipid nanoparticle (LNP) comprises (a) a Cas nuclease, or a nucleic acid encoding the Cas nuclease, and/or (b) a guide RNA, or one or more DNAs encoding the guide RNA. In some embodiments, the Cas nuclease is a Cas9 protein. In some embodiments, the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some embodiments, the nucleic acid encoding the Cas protein is codon-optimized for expression in a mammalian cell. In some embodiments, the nucleic acid encoding the Cas protein is codon-optimized for expression in a human cell. In some embodiments, the nucleic acid encoding the Cas nuclease comprises an mRNA encoding the Cas protein. In some embodiments, the guide RNA is a single guide RNA (sgRNA). In some embodiments, the lipid nanoparticle (LNP) comprises a zinc finger nuclease (ZFN), or a transcription activator-like effector nuclease (TALEN).

In some embodiments, the lipid nanoparticle comprises a cationic lipid, a neutral lipid, a helper lipid, and a stealth lipid. In some embodiments, the neutral lipid is distearoylphosphatidylcholine (DSPC). In some embodiments, the helper lipid is cholesterol. In some embodiments, the stealth lipid is PEG2k-DMG.

In some embodiments, the antigen-binding protein, when not conjugated to a molecular cargo, does not block more than 50% of binding of a human transferrin receptor C-terminal fragment to human holo-transferrin that occurs in the absence of such single chain fragment variable (scFv), antibody or an antigen-binding fragment. In some embodiments, said blocking is as measured in an Enzyme Linked Immunosorbent Assay (ELISA) plate assay wherein human transferrin receptor extracellular domain that is fused to a His6-myc-myc tag is pre-bound to said scFv, antibody or antigen-binding fragment and is then contacted with holo-transferrin which is immobilized to the surface of the plate by binding of an anti-holo-transferrin antibody that is bound to the plate.

In some embodiments, binding of the holo-transferrin and human transferrin receptor extracellular domain in the absence of the antigen-binding protein is measured at a concentration of about 300 pM human transferrin receptor extracellular domain.

In various embodiments, a protein-drug conjugate described herein comprises one or more of the following characteristics: a) Affinity (K_{D}) for binding to human TfR at 25° C. in surface plasmon resonance format of about 41 nM or a higher affinity; b) Affinity (K_{D}) for binding to monkey TfR at 25° C. in surface plasmon resonance format of about 0 nM (no detectable binding) or a higher affinity; c) Ratio of [K_{D} for binding to monkey TfR/K_{D} for binding to human TfR] at 25° C. in surface plasmon resonance format of from 0 to 278; d) Blocks about 3-13% hTfR binding to Human Holo-Tf when in Fab format (IgG1); e) Blocks about 6-13% hTfR binding to Human Holo-Tf when in scFv (V_{K}—V_{H}) format; and/or f) Blocks about 11-26% hTfR binding to Human Holo-Tf when in scFv (V_{H}-V_{L}) format.

In another aspect, provided herein is a pharmaceutical composition comprising a protein-drug conjugate described herein and a pharmaceutically acceptable carrier.

In another aspect, provided herein is a composition or kit comprising a protein-drug conjugate or pharmaceutical composition thereof described herein in association with a further therapeutic agent. In one embodiment, the further therapeutic agent is selected from: alglucosidase alfa, rituximab, methotrexate, Intravenous immunoglobulin (IVIG), avalglucosidase alfa, levalbuterol, an antibiotic, cortisone, prednisone, a bisphosphonate, and palivizumab. In one embodiment, the further therapeutic agent is selected from: a Beta2-adrenergic agonist, a steroid, a bisphosphonate, an infectious disease treatment, a vaccine, and a Pneumococcal vaccine.

In another aspect, provided herein is a complex comprising a protein-drug conjugate described herein bound to a human transferrin receptor polypeptide or antigenic fragment thereof.

In another aspect, provided herein is a method for making a protein-drug conjugate described herein comprising (a) contacting the antigen-binding protein, with a molecular cargo under the conditions favorable for conjugation of the antigen-binding protein to the molecular cargo, and (b) optionally, isolating the protein-drug conjugate produced in step (a). In another aspect provided herein is a protein-drug conjugate which is the product of such a method.

In another aspect, provided herein is a vessel or injection device comprising the protein-drug conjugate described herein.

In another aspect, provided herein is a method for administering a protein-drug conjugate described herein to a subject comprising introducing the protein-drug conjugate into the body of the subject (e.g., brain or muscle). In some embodiments, the protein-drug conjugate is introduced into the body of the subject parenterally (e.g., intravenously).

In some embodiments, the protein-drug conjugate is introduced into the body of the subject via intrathecal, intracerebroventricular, or intraparenchymal injection into the central nervous system.

In another aspect, provided herein is a method for treating or preventing a disease or disorder in a subject in need thereof comprising administering, to the subject, an effective amount of the protein-drug conjugate described herein. In some embodiments, the disease or disorder is a lysosomal storage disease and disorder, a heart disease or disorder, a central nervous system (CNS) disease or disorder, an eye disease or disorder, a brain disease or disorder, a spinal cord disease or disorder, a peripheral nervous system (PNS) disease or disorder, a muscular disease or disorder, a cartilage disease or disorder, a bone growth plate disease or disorder, a kidney disease or disorder, or a blood disease or disorder.

In some embodiments, the disease or disorder is a neurological disease or disorder. In some embodiments, the disease or disorder is lysosomal storage disease, amyloidosis, neuropathy, neurodegenerative disease, seizure, behavioral disorder, leukodystrophy, neuropsychiatric diseases, traumatic brain injury, neurodevelopmental diseases, neuromuscular diseases, ocular disease or disorder, viral or microbial infection, inflammation, ischemia, and cancer.

In some embodiments, the disease or disorder is lysosomal storage disease.

In some embodiments, the disease or disorder is a neurodegenerative disease such as Alzheimer's disease, Huntington's disease, amyotrophic lateral sclerosis, Parkinson's disease, or prion disease. In some embodiments, the molecular cargo of the protein-drug conjugate is an siRNA selected from the group consisting of an siRNA that inhibits the ApoE, MAPT, APP, HTT, SOD1, C9orf72, SNCA, LRRK2, or PRNP gene or a mutant thereof.

In some embodiments, the disease or disorder is a heart disease or disorder. In some embodiments, the heart disease or disorder is heart failure. In some embodiments, the molecular cargo of the protein-drug conjugate is an siRNA selected from the group consisting of an siRNA that inhibits the SLC5A1, SLC16A3, HDAC6, MMP27, MFAP5, FAM64A, BAIAP3, MYH7, TPM1, RBM20, KLHL24, MYL2, or TNNT2 gene or a mutant thereof.

In some embodiments, the disease or disorder is a muscular disease or disorder. In some embodiments, the muscular disease or disorder is myotonic dystrophy, duchenne muscular dystrophy, fascioscapulohumeral muscular dystrophy, facioscapulohumeral muscular dystrophy-type 1, or muscle atrophy. In some embodiments, the molecular cargo of the protein-drug conjugate is an siRNA selected from the group consisting of an siRNA that inhibits the DMPK, CNBP, Dystrophin, or DUX4 gene or a mutant thereof.

In various embodiments, the subject is administered the protein-drug conjugate in association with a further therapeutic agent. In some embodiments, the further therapeutic agent is selected from: alglucosidase alfa, rituximab, methotrexate, Intravenous immunoglobulin (IVIG), avalglucosidase alfa, levalbuterol, an antibiotic, cortisone, prednisone, a bisphosphonate, and palivizumab. In some embodiments, the further therapeutic agent is selected from: a Beta2-adrenergic agonist, a steroid, a bisphosphonate, an infectious disease treatment, a vaccine, and a Pneumococcal vaccine.

In another aspect, provided herein isa method for treating or preventing myotonic dystrophy, duchenne muscular dystrophy, fascioscapulohumeral muscular dystrophy, facioscapulohumeral muscular dystrophy-type 1, in a subject in need thereof comprising administering, to the subject, an effective amount of the protein-drug conjugate described herein. In some embodiments, the molecular cargo of the protein-drug conjugate is an interfering RNA (e.g., siRNA) selected from the group consisting of an interfering RNA (e.g., siRNA) that inhibits the DMPK, CNBP, Dystrophin, or DUX4 gene or a mutant thereof.

In another aspect, the present disclosure provides a method for treating or preventing a neurodegenerative disease such as Alzheimer's disease, Huntington's disease, amyotrophic lateral sclerosis, Parkinson's disease, or prion disease in a subject in need thereof comprising administering, to the subject, an effective amount of the protein-drug conjugate described herein. In some embodiments, the molecular cargo of the protein-drug conjugate is an interfering RNA (e.g., siRNA) selected from the group consisting of an interfering RNA (e.g., siRNA) that inhibits the ApoE, MAPT, APP, HTT, SOD1, C9orf72, SNCA, LRRK2, or PRNP gene or a mutant thereof.

In another aspect, the present disclosure provides a method for treating or preventing a heart disease or disorder such as heart failure in a subject in need thereof comprising administering, to the subject, an effective amount of the protein-drug conjugate described herein. In some embodiments, the molecular cargo of the protein-drug conjugate is an siRNA selected from the group consisting of an siRNA that inhibits the SLC5A1, SLC16A3, HDAC6, MMP27, MFAP5, FAM64A, BAIAP3, MYH7, TPM1, RBM20, KLHL24, MYL2, or TNNT2 gene or a mutant thereof.

In another aspect, provided herein is a method for delivering a molecular cargo to a tissue or cell type in the body of a subject comprising administering, to the subject, an antigen-binding protein that binds specifically to human transferrin receptor or an antigenic-fragment thereof or variant thereof to the subject conjugated to the molecular cargo. In some embodiments, the molecular cargo comprises a polynucleotide molecule, a carrier or a small molecule. In some embodiments, the tissue is brain/spinal cord/CNS; eye; skeletal muscle; adipose tissue; blood/bone marrow; breast; lung/bronchus; colon; uterus; esophagus; heart; kidney; liver; lymph node; ovary; pancreas; placenta; prostate; rectum; skin; peripheral blood mononuclear cell (PBMC); small intestine; spleen; stomach; testis; peripheral nervous system; and/or bone/cartilage/joint. In some embodiments, the cell type and tissue that is associate with the cell type is selected from Table 1-4 herein.

In some embodiments, the method comprises piercing the body of the subject with a needle of a syringe and injecting the antigen-binding protein that binds specifically to transferrin receptor or an antigenic-fragment thereof or variant thereof to the subject conjugated to the molecular cargo into the body of the subject. In some embodiments, the subject suffers from a muscle atrophy condition, metabolic disease, sarcopenia or cachexia.

### BRIEF DESCRIPTION OF THE FIGURES

FIGS. 1A-1C show western blots showing that anti-human TFRC antibody clones deliver GAA to the cerebrum of TfrC^{hum} mice. Each lane=1 mouse. Anti-mouse mTfR:GAA in Wt mice was used as a positive control. Anti-mouse mTfR:GAA in Tfrc^{hum} mice was used as a negative control.

FIG. 2 shows western blots showing that a subset of anti-hTFRC antibody clones deliver mature GAA to the brain parenchyma in scfv:GAA format (delivery by HDD). Anti-mouse mTfR:GAA in Wt mice was used as a positive control. Anti-mouse mTfR:GAA in Tfrc^{hum} mice was used as a negative control.

FIG. 3 shows western blots showing that four selected anti-hTFRC antibody clones deliver mature GAA to the brain parenchyma in scfv:GAA format (AAV8 episomal liver depot gene therapy). Anti-mouse mTfR:GAA in Wt mice was used as a positive control. Anti-mouse mTfR:GAA in Tfrc^{hum} mice was used as a negative control.

FIG. 4 shows western blots showing that three selected episomal AAV8 liver depot anti-hTFRC antibody clones deliver mature GAA to the CNS, heart, and muscle in Gaa^{-/-}/Tfrc^{hum} mice.

FIG. 5 shows that three selected episomal AAV8 liver depot anti-hTFRC antibody clones rescue glycogen storage in CNS, heart, and muscle in Gaa^{-/-}/Tfrc^{hum} mice. Wt untreated mice were a positive control, and Gaa^{-/-} untreated mice were a negative control.

FIGS. 6A-6D show that three selected episomal AAV8 liver depot anti-hTFRC antibody clones rescue glycogen storage in brain thalamus (FIG. 6A), brain cerebral cortex (FIG. 6B), brain hippocampus CA1 (FIG. 6C), and quadricep (FIG. 6D) in Gaa^{-/-}/Tfrc^{hum} mice. Wt untreated mice were a positive control, and Gaa^{-/-} untreated mice were a negative control.

FIG. 7A shows that insertion of anti-hTFRC 12847scfv:GAA delivers mature GAA protein to CNS and muscle of Pompe model mice. FIG. 7B shows that insertion of anti-hTFRC 12847scfv:GAA rescues glycogen storage in CNS and muscle of Pompe model mice. One Way ANOVA *p<0.01; **p<0.001; ***p<0.0001. Untreated Pompe disease model mice and wild type mice were used as controls. Mice injected with a recombinant AAV8 anti-TfR:GAA episomal template were used as a positive control. Mice injected with a recombinant AAV8 anti-TfR:GAA insertion template without LNP-g666 were used as a negative control.

FIG. 8 shows the interaction of Mammarenavirus machupoense GP1 protein (PDB 3KAS), human ferritin (PDB 6GSR), Plasmodium vivax Sal-1 PvRBP2b protein (PDB 6D04), human HFE protein (PDB 1DE4), and human transferrin (PDB 1SUV) molecules superimposed on two TfR molecules in a symmetrical unit. For Mammarenavirus machupoense GP1 protein and human ferritin, only one copy in the symmetrical unit is shown to reduce complexity of the figure for clear view.

FIG. 9 depicts Hydrogen-Deuterium Exchange Mass Spectrometry (HDX) protections for the antibodies tested in HDX-MS experiments can be assigned to 5 regions in TfR (PDB 1SUV).

FIG. 10 illustrates TfR regions protected by REGN17513, a representation of antibodies that cause HDX protections in TfR apical domain that overlap with Mammarenavirus machupoense GP1 protein, human ferritin, and Plasmodium vivax PvRBP2b protein binding sites.

FIG. 11 illustrates TfR regions protected by REGN17510, a representation of antibodies with HDX protections in TfR apical domain that are not shared by other TfR binding partners shown in FIG. 11.

FIG. 12 illustrates TfR regions protected by REGN17515, a representation of antibodies with HDX protections in TfR apical domain that share binding sites with human ferritin and Plasmodium vivax Sal-1 PvRBP2b protein.

FIG. 13 illustrates TfR regions protected by REGN17514, a representation of antibodies with HDX protections in TfR protease-like domain and share binding sites with Plasmodium vivax Sal-1 PvRBP2b protein.

FIG. 14 illustrates TfR regions protected by REGN17508, a representation of antibodies with HDX protections in TfR protease-like domain. This region is not utilized by other TfR interacting molecules shown in FIG. 14.

### DETAILED DESCRIPTION OF THE INVENTION

Provided herein are anti-transferrin receptor (TfR) antigen-binding proteins that are conjugated to a molecular cargo. Such conjugates are useful, for example, for delivery of the molecular cargo to various tissues in the body, including the brain and muscle. For example, anti-TfR protein-drug conjugates exhibiting high affinity to the transferrin receptor and superior blood-brain barrier crossing are provided. Surprisingly, anti-TfR scFvs exhibiting high binding affinity to TfR crossed the BBB more efficiently than that of low affinity binders. This is in contrast to previous findings with mono- and bivalent anti-TFR antibodies, where low affinity antibodies crossed the BBB more effectively. The conjugates described herein have an ability to efficiently deliver molecular cargoes to the brain and muscle and, thus, can be used for treatment of diseases and disorders such as neurological or muscular diseases and disorders.

In accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

A polynucleotide includes DNA and RNA. The present disclosure includes any polynucleotide described herein which is operably linked to a promoter or other expression control sequence.

Transferrin receptor 1 (TfR) is a membrane receptor involved in the control of iron supply to the cell through the binding of transferrin, the major iron-carrier protein. Transferrin receptor 1 is expressed from the TFRC gene. Transferrin receptor 1 may be referred to, herein, as TFRC. This receptor plays a key role in the control of cell proliferation because iron is essential for sustaining ribonucleotide reductase activity, and is the only enzyme that catalyzes the conversion of ribonucleotides to deoxyribonucleotides. Preferably, the TfR is human TfR (hTfR). See e.g., Accession numbers NP_001121620.1; BAD92491.1; and NP_001300894.1.; and e!Ensembl entry: ENSG00000072274. The human transferrin receptor 1 is expressed in several tissues, including but not limited to: cerebral cortex; cerebellum; hippocampus; caudate; parathyroid gland; adrenal gland; bronchus; lung; oral mucosa; esophagus; stomach; duodenum; small intestine; colon; rectum; liver; gallbladder; pancreas; kidney; urinary bladder; testis; epididymis; prostate; vagina; ovary; fallopian tube; endometrium; cervix; placenta; breast; heart muscle; smooth muscle; soft tissue; skin; appendix; lymph node; tonsil; and bone marrow. See also tissues and cell types of Table 1-4 herein. A related transferrin receptor is transferrin receptor 2 (TfR2). Human transferrin receptor 2 bears about 45% sequence identity to human transferrin receptor 1. Trinder & Baker, Transferrin receptor 2: a new molecule in iron metabolism. Int J Biochem Cell Biol. 2003 March; 35(3):292-6. Unless otherwise stated, transferrin receptor as used herein generally refers to transferrin receptor 1 (e.g., human transferrin receptor 1).

Human Transferrin (Tf) is a single chain, 80 kDa member of the anion-binding superfamily of proteins. Transferrin is a 698 amino acid precursor that is divided into a 19 aa signal sequence plus a 679 aa mature segment that typically contains 19 intrachain disulfide bonds. The N- and C-terminal flanking regions (or domains) bind ferric iron through the interaction of an obligate anion (e.g., bicarbonate) and four amino acids (His, Asp, and two Tyr). Apotransferrin (or iron-free) will initially bind one atom of iron at the C-terminus, and this is followed by subsequent iron binding by the N-terminus to form holotransferrin (diferric Tf, Holo-Tf). Through its C-terminal iron-binding domain, holotransferrin will interact with the TfR on the surface of cells where it is internalized into acidified endosomes. Iron dissociates from the Tf molecule within these endosomes, and is transported into the cytosol as ferrous iron. In addition to TfR, transferrin is reported to bind to cubulin, IGFBP3, microbial iron-binding proteins and liver-specific TfR2.

The blood-brain barrier (BBB) is located within the microvasculature of the brain, and it regulates passage of molecules from the blood to the brain. Burkhart et al., Accessing targeted nanoparticles to the brain: the vascular route. Curr Med Chem. 2014; 21(36):4092-9. The transcellular passage through the brain capillary endothelial cells can take place via 1) cell entry by leukocytes; 2) carrier-mediated influx of e.g., glucose by glucose transporter 1 (GLUT-1), amino acids by e.g., the L-type amino acid transporter 1 (LAT-1) and small peptides by e.g., organic anion-transporting peptide-B (OATP-B); 3) paracellular passage of small hydrophobic molecules; 4) adsorption-mediated transcytosis of e.g., albumin and cationized molecules; 5) passive diffusion of lipid soluble, non-polar solutes, including CO₂ and O₂; and 5) receptor-mediated transcytosis of e.g., insulin by the insulin receptor and Tf by the TfR. Johnsen et al., Targeting the transferrin receptor for brain drug delivery, Prog Neurobiol. 2019 October; 181:101665.

### Anti-Human Transferrin Receptor Antigen-Binding Protein Conjugates

Provided herein are anti-hTfR protein-drug conjugates. An anti-hTfR protein-drug conjugate comprises an optional signal peptide, connected to an antigen-binding protein (e.g., an antibody or an antigen-binding fragment of an antibody such as an Fab or scFv) that binds specifically to transferrin receptor, preferably, human transferrin receptor 1 (hTfR) which is conjugated (optionally by a linker) to molecular cargo. The anti-hTfR antigen-binding proteins described herein efficiently cross the blood-brain barrier (BBB) and can, thereby, deliver the conjugated molecular cargo to the brain.

An antigen-binding protein that specifically binds to transferrin receptor and protein-drug conjugates thereof, for example, a tag such as His₆ and/or myc (e.g., human transferrin receptor (e.g., REGN2431) or monkey transferrin receptor (e.g., REGN2054)) binds at about 25° C., e.g., in a surface plasmon resonance assay, with a K_{D} of about 20 nM or a higher affinity. Such an antigen-binding protein may be referred to as “anti-TfR”.

The term “conjugate” means a body in which two substances are linked covalently, or non-covalently. The term “covalently linked” refers to a characteristic of at least two molecules being linked together by way of one or more covalent bond(s). In various embodiments, two molecules can be covalently linked together by a single bond, e.g., a disulfide bridge or a disulfide bond, that operates as a linker between the molecules. In some embodiments, two or more molecules may be covalently linked together by way of a molecule that operates as a linker that joins the at least two molecules together via multiple covalent bonds. In certain embodiments, a linker can be a cleavable linker or a non-cleavable linker. In the conjugate, the two substances may be linked directly or may be linked via a linker. In certain embodiments, one of the two substances is an antigen-binding protein, e.g., an antibody or antigen-binding fragment thereof, and the other is a drug (e.g., a polynucleotide, or a liposome or LNP disclosed herein). In certain embodiments, the linker may be a cleavable linker or may be a non-cleavable linker.

As used herein, the term “antibody-drug conjugate” or “ADC” means a conjugate of an antibody or antigen-binding fragment thereof with a drug (e.g., a polynucleotide, or a liposome or LNP disclosed herein). The affinity to an antigen is imparted to a drug by linking an antibody or antigen-binding fragment thereof with the drug (e.g., a polynucleotide, or a liposome or LNP disclosed herein), thereby increasing the efficiency of delivering the drug to a target site in vivo.

In an embodiment, the assignment of amino acids to each framework or CDR domain in an immunoglobulin is in accordance with the definitions of Sequences of Proteins of Immunological Interest, Kabat et al.; National Institutes of Health, Bethesda, Md.; 5^{th} ed.; NIH Publ. No. 91-3242 (1991); Kabat (1978) Adv. Prot. Chem. 32:1-75; Kabat et al., (1977) J. Biol. Chem. 252:6609-6616; Chothia, et al., (1987) J Mol. Biol. 196:901-917 or Chothia, et al., (1989) Nature 342: 878-883. Thus, also included herein are antibodies and antigen-binding fragments including the CDRs of a V_{H} and the CDRs of a V_{L}, which V_{H} and V_{L} comprise amino acid sequences as set forth herein (see e.g., sequences of Table 1-1, or a variant thereof), wherein the CDRs are as defined according to Kabat and/or Chothia.

Protein-drug conjugates described herein include antibodies that bind specifically to the human transferrin receptor 1. The term “antibody”, as used herein, refers to immunoglobulin molecules comprising four polypeptide chains, two heavy chains (HCs) and two light chains (LCs), inter-connected by disulfide bonds. In an embodiment, each antibody heavy chain (HC) comprises a heavy chain variable region (“HCVR” or “V_{H}”) (e.g., comprising SEQ ID NO: 2; 462; 12; 463; 22; 464; 32; 42; 52; 467; 62; 492; 72; 470; 82; 92; 472; 102; 112; 473; 122; 132; 142; 475; 152; 162; 477; 172; 182; 478; 192; 480; 202; 481; 212; 222; 232; 242; 252; 482; 262; 272; 282; 292; 302; 483 and/or 312 or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 7; 17; 27; 37; 465; 47; 466; 57; 468; 67; 469; 77; 471; 87; 97; 107; 117; 474; 127; 137; 147; 476; 157; 167; 177; 187; 479; 197; 207; 217; 227; 237; 247; 257; 267; 277; 287; 297; 307; 527; 317 and/or 484 or a variant thereof) and a light chain constant region (e.g., human kappa or human lambda). The V_{H} and V_{L} regions can be further subdivided into regions of hypervariability, termed complementarity determining regions (CDR), interspersed with regions that are more conserved, termed framework regions (FR). Each V_{H} and V_{L} comprises three CDRs and four FRs. Anti-TfR antibodies described herein can also be conjugated to a molecular cargo.

An anti-TfR antigen-binding protein described herein may be an antigen-binding fragment of an antibody which may be conjugated to a molecular cargo. The terms “antigen-binding portion” or “antigen-binding fragment” of an antibody, as used herein, refers to an immunoglobulin molecule that binds antigen but that does not include all of the sequences of a full antibody (preferably, the full antibody is an IgG). Non-limiting examples of antigen-binding fragments include: (i) Fab fragments; (ii) F(ab′)₂ fragments; (iii) Fd fragments; (iv) Fv fragments; (v) single-chain Fv (scFv) molecules; and (vi) dAb fragments; consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, one-armed antibodies, domain-deleted antibodies, chimeric antibodies, CDR-grafted antibodies, diabodies, triabodies, tetrabodies, minibodies and small modular immunopharmaceuticals (SMIPs), are also encompassed within the expression “antigen-binding fragment,” as used herein.

In some embodiments, an anti-TfR protein-drug conjugate described herein may comprise an scFv which is conjugated to a molecular cargo. An scFv (single chain fragment variable) has variable regions of heavy (V_{H}) and light (V_{L}) domains (in either order), which, preferably, are joined together by a flexible linker (e.g., peptide linker). The length of the flexible linker used to link both of the V regions may be important for yielding the correct folding of the polypeptide chain. Previously, it has been estimated that the peptide linker must span 3.5 nm (35A) between the carboxy terminus of the variable domain and the amino terminus of the other domain without affecting the ability of the domains to fold and form an intact antigen-binding site (Huston et al., Protein engineering of single-chain Fv analogs and fusion proteins. Methods in Enzymology. 1991; 203:46-88). In an embodiment, the linker comprises an amino acid sequence of such length to separate the variable domains by about 3.5 nm. In an embodiment of the invention, an anti-TfR scFv-drug conjugate includes an scFv comprising the arrangement of variable regions as follows LCVR-HCVR or HCVR-LCVR, wherein the HCVR and LCVR are optionally connected by a linker and the scFv is connected, optionally by a linker, to a molecular cargo (e.g., LCVR-(Gly₄Ser)₃-HCVR-molecular cargo; or LCVR-(Gly₄Ser)₃-HCVR-molecular cargo).

In some embodiments, an anti-TfR protein-drug conjugate described herein may comprise a Fab which is conjugated to a molecular cargo.

In some embodiments, an anti-TfR protein-drug conjugate described herein comprise a bivalent antibody which is conjugated to a molecular cargo.

In some embodiments, an anti-TfR protein-drug conjugate described herein comprises a monovalent or “one-armed” antibody which is conjugated to a molecular cargo. The monovalent or “one-armed” antibodies as used herein refer to immunoglobulin proteins comprising a single variable domain. For example, the one-armed antibody may comprise a single variable domain within a Fab wherein the Fab is linked to at least one Fc fragment. In certain embodiments, the one-armed antibody comprises: (i) a heavy chain comprising a heavy chain constant region and a heavy chain variable region, (ii) a light chain comprising a light chain constant region and a light chain variable region, and (iii) a polypeptide comprising a Fc fragment or a truncated heavy chain. In certain embodiments, the Fc fragment or a truncated heavy chain comprised in the separate polypeptide is a “dummy Fc,” which refers to an Fc fragment that is not linked to an antigen binding domain. The one-armed antibodies described herein may comprise any of the HCVR/LCVR pairs or CDR amino acid sequences as set forth in Table 1-1 herein. One-armed antibodies comprising a full-length heavy chain, a full-length light chain and an additional Fc domain polypeptide can be constructed using standard methodologies (see e.g., WO2010151792, which is incorporated herein by reference in its entirety), wherein the heavy chain constant region differs from the Fc domain polypeptide by at least two amino acids (e.g., H95R and Y96F according to the IMGT exon numbering system; or H435R and Y436F according to the EU numbering system). Such modifications are useful in purification of the monovalent antibodies (see WO2010151792).

An antigen-binding fragment of an antibody will, in an embodiment, comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}-CH1; (ii) V_{H}-CH2; (iii) V_{H}-CH3; (iv) V_{H}-CH1-CH2; (v) V_{H}-CH1-CH2-CH3; (vi) V_{H}-CH2-CH3; (vii) V_{H}-CL; (viii) V_{L}-CH1; (ix) V_{L}-CH2; (x) V_{L}-CH3; (xi) V_{L}-CH1-CH2; (xii) VL-CH1-CH2-CH3; (xiii) V_{L}-CH2-CH3; and (xiv) V_{L}-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)). The present disclosure includes an antigen-binding fragment of an antigen-binding protein such as an antibody set forth herein.

Antigen-binding proteins (e.g., antibodies and antigen-binding fragments) may be monospecific or multi-specific (e.g., bispecific). Multispecific antigen-binding proteins are discussed further herein. The present disclosure includes monospecific as well as multispecific (e.g., bispecific) antigen-binding fragments comprising one or more variable domains from an antigen-binding protein that is specifically set forth herein.

The term “specifically binds” or “binds specifically” refers to those antigen-binding proteins (e.g., antibodies or antigen-binding fragments thereof) having a binding affinity to an antigen, such as human TfR protein, mouse TfR protein or monkey TfR protein, expressed as K_{D}, of at least about 10-9 M (e.g., 0.01, 0.1, 0.2, 0.3, 0.4, 0.5, 0.6, 0.7, 0.8, 0.9 or 1.0 nM), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA. The present disclosure includes antigen-binding proteins that specifically bind to TfR protein. “Anti-TfR” refers to an antigen-binding protein (or other molecule), for example an antibody or antigen-binding fragment thereof, that binds specifically to TfR.

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules (e.g., minor or insignificant amounts of impurity may remain) or to an absence of water, buffers, or salts or to components of a pharmaceutical formulation that includes the antigen-binding proteins (e.g., antibodies or antigen-binding fragments).

The present disclosure includes antigen-binding proteins, e.g., antibodies or antigen-binding fragments, that bind to the same epitope as an antigen-binding protein described herein.

An antigen is a molecule, such as a peptide (e.g., TfR or a fragment thereof (an antigenic fragment)), to which, for example, an antibody or antigen-binding fragment thereof binds. The specific region on an antigen that an antibody recognizes and binds to is called the epitope. Antigen-binding proteins (e.g., antibodies) described herein that specifically bind to such antigens are part of the present disclosure.

The term “epitope” refers to an antigenic determinant (e.g., on TfR) that interacts with a specific antigen-binding site of an antigen-binding protein, e.g., a variable region of an antibody, known as a paratope. A single antigen may have more than one epitope. Thus, different antibodies may bind to different areas on an antigen and may have different biological effects. The term “epitope” may also refer to a site on an antigen to which B and/or T cells respond and/or to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes to which antigen-binding proteins described herein bind may be included in fragments of TfR, for example the extracellular domain thereof. Antigen-binding proteins (e.g., antibodies) described herein that bind to such epitopes are also contemplated.

Methods for determining the epitope of an antigen-binding protein, e.g., antibody or fragment or polypeptide, include alanine scanning mutational analysis, peptide blot analysis (Reineke (2004) Methods Mol. Biol. 248: 443-63), peptide cleavage analysis, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antigen-binding protein (e.g., antibody or fragment or polypeptide) interacts is hydrogen/deuterium exchange detected by mass spectrometry.

See, e.g., Ehring (1999) Analytical Biochemistry 267: 252-259; Engen and Smith (2001) Anal. Chem. 73: 256A-265A.

The present disclosure includes antigen-binding proteins that compete for binding to a TfR epitope as discussed herein, with an antigen-binding protein described herein,. The term “competes” as used herein, refers to an antigen-binding protein (e.g., antibody or antigen-binding fragment thereof) that binds to an antigen (e.g., TfR) and inhibits or blocks the binding of another antigen-binding protein (e.g., antibody or antigen-binding fragment thereof) to the antigen. Unless otherwise stated, the term also includes competition between two antigen-binding proteins e.g., antibodies, in both orientations, i.e., a first antibody that binds antigen and blocks binding by a second antibody and vice versa. Thus, in an embodiment, competition occurs in one such orientation. In certain embodiments, the first antigen-binding protein (e.g., antibody) and second antigen-binding protein (e.g., antibody) may bind to the same epitope. Alternatively, the first and second antigen-binding proteins (e.g., antibodies) may bind to different, but, for example, overlapping or non-overlapping epitopes, wherein binding of one inhibits or blocks the binding of the second antibody, e.g., via steric hindrance. Competition between antigen-binding proteins (e.g., antibodies) may be measured by methods known in the art, for example, by a real-time, label-free bio-layer interferometry assay. Also, binding competition between TfR-binding proteins (e.g., monoclonal antibodies (mAbs)) can be determined using a real time, label-free bio-layer interferometry assay on an Octet RED384 biosensor (Pall ForteBio Corp.).

Typically, an antibody or antigen-binding fragment described herein which is modified in some way retains the ability to specifically bind to TfR, e.g., retains at least 10% of its TfR binding activity (when compared to the parental antibody) when that activity is expressed on a molar basis. Preferably, an antibody or antigen-binding fragment described herein retains at least 20%, 50%, 70%, 80%, 90%, 95% or 100% or more of the TfR binding affinity as the parental antibody. It is also intended that an antibody or antigen-binding fragment described herein may include conservative or non-conservative amino acid substitutions (referred to as “conservative variants” or “function conserved variants” of the antibody) that do not substantially alter its biologic activity.

An anti-TfR antigen-binding protein described herein may be a monoclonal antibody or an antigen-binding fragment of a monoclonal antibody which may be conjugated to a molecular cargo. The present disclosure includes monoclonal anti-TfR antigen-binding proteins, e.g., antibodies and antigen-binding fragments thereof, as well as monoclonal compositions comprising a plurality of isolated monoclonal antigen-binding proteins. The term “monoclonal antibody” or “mAb”, as used herein, refers to a member of a population of substantially homogeneous antibodies, i.e., the antibody molecules comprising the population are identical in amino acid sequence except for possible naturally occurring mutations that may be present in minor amounts. A “plurality” of such monoclonal antibodies and fragments in a composition refers to a concentration of identical (i.e., as discussed above, in amino acid sequence except for possible naturally occurring mutations that may be present in minor amounts) antibodies and fragments which is above that which would normally occur in nature, e.g., in the blood of a host organism such as a mouse or a human.

In an embodiment, an anti-TfR antigen-binding protein, e.g., antibody or antigen-binding fragment (which may be conjugated to a molecular cargo) comprises a heavy chain constant domain, e.g., of the type IgA (e.g., IgA1 or IgA2), IgD, IgE, IgG (e.g., IgG1, IgG2, IgG3 and IgG4) or IgM. In an embodiment, an antigen-binding protein, e.g., antibody or antigen-binding fragment, comprises a light chain constant domain, e.g., of the type kappa or lambda. In an embodiment, a V_{H} as set forth herein is linked to a human heavy chain constant domain (e.g., IgG) and a V_{L} as set forth herein is linked to a human light chain constant domain (e.g., kappa). The present disclosure includes antigen-binding proteins comprising the variable domains set forth herein, which are linked to a heavy and/or light chain constant domain, e.g., as set forth herein.

The present disclosure includes human anti-TfR antigen-binding proteins which may be conjugated to a molecular cargo. The term “human” antigen-binding protein, such as an antibody or antigen-binding fragment, as used herein, includes antibodies and fragments having variable and constant regions derived from human germline immunoglobulin sequences whether in a human cell or grafted into a non-human cell, e.g., a mouse cell. See e.g., U.S. Pat. Nos. 8,502,018; 6,596,541 or 5,789,215. The anti-TfR human mAbs described herein may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse) have been grafted onto human FR sequences. The term includes antibodies recombinantly produced in a non-human mammal or in cells of a non-human mammal. The term is not intended to include natural antibodies directly isolated from a human subject. The present disclosure includes human antigen-binding proteins (e.g., antibodies or antigen-binding fragments thereof described herein).

The present disclosure includes anti-TfR chimeric antigen-binding proteins, e.g., antibodies and antigen-binding fragments thereof (which may be conjugated to a molecular cargo), and methods of use thereof. As used herein, a “chimeric antibody” is an antibody having the variable domain from a first antibody and the constant domain from a second antibody, where the first and second antibodies are from different species. (see e.g., U.S. Pat. No. 4,816,567; and Morrison et al., (1984) Proc. Natl. Acad. Sci. USA 81: 6851-6855). The present disclosure includes chimeric antibodies comprising the variable domains which are set forth herein and a non-human constant domain.

The term “recombinant” anti-TfR antigen-binding proteins, such as antibodies or antigen-binding fragments thereof (which may be conjugated to a molecular cargo), refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a cell (e.g., CHO cells) such as a cellular expression system or isolated from a recombinant combinatorial human antibody library. The present disclosure includes recombinant antigen-binding proteins, such as antibodies and antigen-binding fragments as set forth herein.

An antigen-binding fragment of an antibody will, in an embodiment, comprise less than a full antibody but still binds specifically to antigen, e.g., TfR, e.g., including at least one variable domain. The variable domain may be of any size or amino acid composition and will generally comprise at least one (e.g., 3) CDR(s), which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} and/or V_{L} domain which are bound non-covalently.

A “variant” of a polypeptide, such as an immunoglobulin chain, refers to a polypeptide comprising an amino acid sequence that is at least about 70-99.9% (e.g., at least 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein (e.g., any of SEQ ID nOs: 2; 3; 4; 5; 7; 8; 9; 10; 12; 13; 14; 15; 17; 18; 19; 20; 22; 23; 24; 25; 27; 28; 29; 30; 32; 33; 34; 35; 37; 38; 39; 40; 42; 43; 44; 45; 47; 48; 49; 50; 52; 53; 54; 55; 57; 58; 59; 60; 62; 63; 64; 65; 67; 68; 69; 70; 72; 73; 74; 75; 77; 78; 79; 80; 82; 83; 84; 85; 87; 88; 89; 90; 92; 93; 94; 95; 97; 98; 99; 100; 102; 103; 104; 105; 107; 108; 109; 110; 112; 113; 114; 115; 117; 118; 119; 120; 122; 123; 124; 125; 127; 128; 129; 130; 132; 133; 134; 135; 137; 138; 139; 140; 142; 143; 144; 145; 147; 148; 149; 150; 152; 153; 154; 155; 157; 158; 159; 160; 162; 163; 164; 165; 167; 168; 169; 170; 172; 173; 174; 175; 177; 178; 179; 180; 182; 183; 184; 185; 187; 188; 189; 190; 192; 193; 194; 195; 197; 198; 199; 200; 202; 203; 204; 205; 207; 208; 209; 210; 212; 213; 214; 215; 217; 218; 219; 220; 222; 223; 224; 225; 227; 228; 229; 230; 232; 233; 234; 235; 237; 238; 239; 240; 242; 243; 244; 245; 247; 248; 249; 250; 252; 253; 254; 255; 257; 258; 259; 260; 262; 263; 264; 265; 267; 268; 269; 270; 272; 273; 274; 275; 277; 278; 279; 280; 282; 283; 284; 285; 287; 288; 289; 290; 292; 293; 294; 295; 297; 298; 299; 300; 302; 303; 304; 305; 307; 527; 308; 309; 310; 312; 313; 314; 315; 317; 318; 319; 320; and 328-423); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment) and/or comprising the amino acid sequence but having one or more (e.g., 1, 2, 3, 4, 5, 6, 7, 8, 9 or 10) mutations (e.g., point mutation, insertion, truncation, and/or deletion).

Moreover, a variant of a polypeptide may include a polypeptide such as an immunoglobulin chain which may include the amino acid sequence of the reference polypeptide whose amino acid sequence is specifically set forth herein but for one or more (e.g., 1, 2, 3, 4, 5, 6, 7, 8, 9 or 10) mutations, e.g., one or more missense mutations (e.g., conservative substitutions), non-sense mutations, deletions, or insertions. For example, the present disclosure includes TfR-binding proteins which include an immunoglobulin light chain (or V_{L}) variant comprising the amino acid sequence set forth in SEQ ID NO: 7, 17, 27, 37, 465, 47, 466, 57, 468, 67, 469, 77, 471, 87, 97, 107, 117, 474, 127, 137, 147, 476, 157, 167, 177, 187, 479, 197, 207, 217, 227, 237, 247, 257, 267, 277, 287, 297, 307, 527, 317, 484 but having one or more of such mutations and/or an immunoglobulin heavy chain (or V_{H}) variant comprising the amino acid sequence set forth in SEQ ID NO: 2, 462, 12, 463, 22, 464, 32, 42, 52, 467, 62, 492, 72, 470, 82, 92, 472, 102, 112, 473, 122, 132, 142, 475, 152, 162, 477, 172, 182, 478, 192, 480, 202, 481, 212, 222, 232, 242, 252, 482, 262, 272, 282, 292, 302, 483, 312 but having one or more of such mutations. In an embodiment, a TfR-binding protein includes an immunoglobulin light chain variant comprising CDR-L1, CDR-L2 and CDR-L3 wherein one or more (e.g., 1 or 2 or 3) of such CDRs has one or more of such mutations (e.g., conservative substitutions) and/or an immunoglobulin heavy chain variant comprising CDR-H1, CDR-H2 and CDR-H3 wherein one or more (e.g., 1 or 2 or 3) of such CDRs has one or more of such mutations (e.g., conservative substitutions).

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

A “conservatively modified variant” or a “conservative substitution”, e.g., of an immunoglobulin chain set forth herein, refers to a variant wherein there is one or more substitutions of amino acids in a polypeptide with other amino acids having similar characteristics (e.g., charge, side-chain size, hydrophobicity/hydrophilicity, backbone conformation and rigidity, etc.). Such changes can frequently be made without significantly disrupting the biological activity of the antibody or fragment. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to significantly disrupt biological activity. The present disclosure includes TfR-binding proteins comprising such conservatively modified variant immunoglobulin chains.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Alternatively, a conservative replacement is any change having a positive value in the PAM250 log-likelihood matrix disclosed in Gonnet et al. (1992) Science 256: 1443-45.

Antibodies and antigen-binding fragments described herein comprise immunoglobulin chains including the amino acid sequences specifically set forth herein (and variants thereof) as well as cellular and in vitro post-translational modifications to the antibody or fragment. For example, the present disclosure includes antibodies and antigen-binding fragments thereof that specifically bind to TfR comprising heavy and/or light chain amino acid sequences set forth herein as well as antibodies and fragments wherein one or more asparagine, serine and/or threonine residues is glycosylated, one or more asparagine residues is deamidated, one or more residues (e.g., Met, Trp and/or His) is oxidized, the N-terminal glutamine is pyroglutamate (pyroE) and/or the C-terminal lysine or other amino acid is missing.

In an embodiment, an anti-hTfR protein-drug conjugates (e.g., in scFv, Fab, or other antibody or antigen-binding fragment thereof format) can exhibit one or more of the following characteristics:
Affinity (K_{D}) for binding to human TfR at 25° C. in surface plasmon resonance format of about 41 nM or a higher affinity (e.g., about 1 or 0.1 nM or about 0.18 to about 1.2 nM, or higher);
Affinity (K_{D}) for binding to monkey TfR at 25° C. in surface plasmon resonance format of about 0 nM (no detectable binding) or a higher affinity (e.g., about 20 nM or higher);
Ratio of K_{D} for binding to monkey TfR/human TfR at 25° C. in surface plasmon resonance format of from 0 to 278 (e.g., about 17 or 18);
Blocks about 3, 5, 10 or 13% hTfR (e.g., Hmm-hTFRC such as REGN2431) binding to Human Holo-Tf when in Fab format (IgG1), e.g., no more than about 45% blocking;
a Blocks about 6, 8, 10 or 13% hTfR (e.g., Hmm-hTFRC such as REGN243) binding to Human Holo-Tf when in scFv (V_{K}—V_{H}) format, e.g., no more than about 45% blocking;
Blocks about 11, 17, 23 or26% hTfR (e.g., Hmm-hTFRC such as REGN243) binding to Human Holo-Tf when in scFv (V_{H}-V_{L}) format, e.g., no more than about 45% blocking;
Tfrc^{hum} or Tfrc^{hum/hum} are homozygous knock-in mice.

The amino acid sequences of domains in anti-human transferrin receptor antigen-binding proteins of conjugates described herein are summarized below in Table 1-1. For example, anti-human transferrin receptor 1 antibodies and antigen-binding fragments thereof (e.g., scFvs and Fabs) comprising the HCVR and LCVR of the molecules in Table 1-1; or comprising the CDRs thereof, conjugated to a molecular cargo, are included herein.

TABLE 1-1
SEQ ID NOs of Domains in Antibodies, Antigen-binding Fragments (e.g.,
Fabs or scFv Molecules) in Described Protein-Drug Conjugates.

| | anti-hTfR | | | | | | | | |
|---|---|---|---|---|---|---|---|---|---|
| # | Molecule | HCVR | HCDR1 | HCDR2 | HCDR3 | LCVR | LCDR1 | LCDR2 | LCDR3 |
| 1 | 31874B | 2 or 462 | 3 | 4 | 5 | 7 | 8 | 9 | 10 |
| 2 | 31863B | 12 or 463 | 13 | 14 | 15 | 17 | 18 | 19 | 20 |
| 3 | 69348 | 22 or 464 | 23 | 24 | 25 | 27 | 28 | 29 | 30 |
| 4 | 69340 | 32 | 33 | 34 | 35 | 37 or 465 | 38 | 39 | 40 |
| 5 | 69331 | 42 | 43 | 44 | 45 | 47 or 466 | 48 | 49 | 50 |
| 6 | 69332 | 52 or 467 | 53 | 54 | 55 | 57 or 468 | 58 | 59 | 60 |
| 7 | 69326 | 62 or 492 | 63 | 64 | 65 | 67 or 469 | 68 | 69 | 70 |
| 8 | 69329 | 72 or 470 | 73 | 74 | 75 | 77 or 471 | 78 | 79 | 80 |
| 9 | 69323 | 82 | 83 | 84 | 85 | 87 | 88 | 89 | 90 |
| 10 | 69305 | 92 or 472 | 93 | 94 | 95 | 97 | 98 | 99 | 100 |
| 11 | 69307 | 102 | 103 | 104 | 105 | 107 | 108 | 109 | 110 |
| 12 | 12795B | 112 or 473 | 113 | 114 | 115 | 117 or 474 | 118 | 119 | 120 |
| 13 | 12798B | 122 | 123 | 124 | 125 | 127 | 128 | 129 | 130 |
| 14 | 12799B | 132 | 133 | 134 | 135 | 137 | 138 | 139 | 140 |
| 15 | 12801B | 142 or 475 | 143 | 144 | 145 | 147 or 476 | 148 | 149 | 150 |
| 16 | 12802B | 152 | 153 | 154 | 155 | 157 | 158 | 159 | 160 |
| 17 | 12808B | 162 or 477 | 163 | 164 | 165 | 167 | 168 | 169 | 170 |
| 18 | 12812B | 172 | 173 | 174 | 175 | 177 | 178 | 179 | 180 |
| 19 | 12816B | 182 or 478 | 183 | 184 | 185 | 187 or 479 | 188 | 189 | 190 |
| 20 | 12833B | 192 or 480 | 193 | 194 | 195 | 197 | 198 | 199 | 200 |
| 21 | 12834B | 202 or 481 | 203 | 204 | 205 | 207 | 208 | 209 | 210 |
| 22 | 12835B | 212 | 213 | 214 | 215 | 217 | 218 | 219 | 220 |
| 23 | 12847B | 222 | 223 | 224 | 225 | 227 | 228 | 229 | 230 |
| 24 | 12848B | 232 | 233 | 234 | 235 | 237 | 238 | 239 | 240 |
| 25 | 12843B | 242 | 243 | 244 | 245 | 247 | 248 | 249 | 250 |
| 26 | 12844B | 252 or 482 | 253 | 254 | 255 | 257 | 258 | 259 | 260 |
| 27 | 12845B | 262 | 263 | 264 | 265 | 267 | 268 | 269 | 270 |
| 28 | 12839B | 272 | 273 | 274 | 275 | 277 | 278 | 279 | 280 |
| 29 | 12841B | 282 | 283 | 284 | 285 | 287 | 288 | 289 | 290 |
| 30 | 12850B | 292 | 293 | 294 | 295 | 297 | 298 | 299 | 300 |
| 31 | 69261 | 302 or 483 | 303 | 304 | 305 | 307 or 527 | 308 | 309 | 310 |
| 32 | 69263 | 312 | 313 | 314 | 315 | 317 or 484 | 318 | 319 | 320 |

31874B
HCVR (V_{H}) Nucleotide Sequence
(SEQ ID NO: 1)
GAGGTGCAGCTGGTGGAGTCTGGGGGAGGCTTGGTACAGCCTGGGGGGTCCCTGAGACTCTCCTGTGCAGCCTCT
GGATTCGCCTTTAGCAGCTATGCCATGACCTGGGTCCGACAGGCTCCAGGGAAGGGGCTGGAGTGGGTCTCAGTT
ATCAGTGGTACTGGTGGTAGTACATACTACGCAGACTCCGTGAAGGGCCGGTTCACCATCTCCAGAGACAATTCC
AAGAACACGCTGTATCTACAAATGAACAGCCTGAGAGCCGAGGACACGGCCGTATATTACTGTGCGAAAGGGGGA
GCAGCTCGTAGAATGGAATACTTCCAGTACTGGGGCCAGGGCACCCTGGTCACCGTCTCCTCA
HCVR (V_{H}) Amino Acid Sequence
(SEQ ID NO: 2)
EVQLVESGGGLVQPGGSLRLSCAASGFAFSSYAMTWVRQAPGKGLEWVSVISGTGGSTYYADSVKGRFTISRDNS
KNTLYLQMNSLRAEDTAVYYCAKGGAARRMEYFQYWGQGTLVTVSS
or
(SEQ ID NO: 462)
EVQLVESGGGLVQPGGSLRLSCAASGFAFSSYAMTWVRQAPGKGLEWVSVISGTGGSTYYADSVKGRFTISRDNS
KNTLYLQMNSLRAEDTAVYYCAKGGAARRMEYFQYWGQGTTVTVSS
HCDR1:
(SEQ ID NO: 3)
GFAFSSYA
HCDR2:
(SEQ ID NO: 4)
ISGTGGST
HCDR3:
(SEQ ID NO: 5)
AKGGAARRMEYFQY
LCVR (V_{L}) Nucleotide Sequence
(SEQ ID NO: 6)
GACATCCAGATGACCCAGTCTCCATCCTCCCTGTCTGCATCTGTAGGAGACAGAGTCACCATCACTTGCCGGGCG
AGTCAGGGCATTAGCAATTATTTAGCCTGGTATCAGCAGAAACCAGGGAAAGTTCCTAACCTCCTTATCTATGCT
GCATCCACTTTGCAATCAGGGGTCCCATCTCGATTCAGTGGCAGTGGATCTGGGACAGATTTCACTCTCACCATC
AGCAGCCTGCAGCCTGAAGATGTTGCAACTTATTACTGTCAAAAGTATAACAGTGCCCCTCTCACTTTCGGCGGA
GGGACCAAGGTGGAGATCAAA
LCVR (V_{L}) Amino Acid Sequence
(SEQ ID NO: 7)
DIQMTQSPSSLSASVGDRVTITCRASQGISNYLAWYQQKPGKVPNLLIYAASTLQSGVPSRFSGSGSGTDFTLTI
SSLQPEDVATYYCQKYNSAPLTFGGGTKVEIK
LCDR1:
(SEQ ID NO: 8)
QGISNY
LCDR2:
(SEQ ID NO: 9)
AAS
LCDR3:
(SEQ ID NO: 10)
QKYNSAPLT
31863B
HCVR (V_{H}) Nucleotide Sequence
(SEQ ID NO: 11)
GAGGTGCAGCTGGTGGAGTCTGGGGGAGGCTTGGTACAGCCTGGGGGGTCCCTGAGACTCTCCTGTGCAGCCTCT
GGATTCACCTTTAACAGCTATGCCATGACCTGGGTCCGCCAGGCTCCAGGGAAGGGGCTGGAGTGGGTCTCATTT
ATTGGTGGTAGTACTGGTAACACATACTACGCAGGCTCCGTGAAGGGCCGGTTCACCATCTCCAGCGACAATTCC
AAGAAGACGCTGTATCTGCAAATGAACAGCCTGAGAGCCGAGGACACGGCCGTATATTACTGTGCGAAAGGGGGA
GCAGCTCGTAGAATGGAATACTTCCAGCACTGGGGCCAGGGCACCCTGGTCACCGTCTCCTCA
HCVR (V_{H}) Amino Acid Sequence
(SEQ ID NO: 12)
EVQLVESGGGLVQPGGSLRLSCAASGFTFNSYAMTWVRQAPGKGLEWVSFIGGSTGNTYYAGSVKGRFTISSDNS
KKTLYLQMNSLRAEDTAVYYCAKGGAARRMEYFQHWGQGTLVTVSS
or
(SEQ ID NO: 463)
EVQLVESGGGLVQPGGSLRLSCAASGFTFNSYAMTWVRQAPGKGLEWVSFIGGSTGNTYYAGSVKGRFTISSDNS
KKTLYLQMNSLRAEDTAVYYCAKGGAARRMEYFQHWGQGTTVTVSS
HCDR1:
(SEQ ID NO: 13)
GFTENSYA
HCDR2:
(SEQ ID NO: 14)
IGGSTGNT
HCDR3:
(SEQ ID NO: 15)
AKGGAARRMEYFQH
LCVR (V_{L}) Nucleotide Sequence
(SEQ ID NO: 16)
GACATCCAGATGACCCAGTCTCCATCCTCCCTGTCTGCATCTATAGGAGACAGAGTCACCATCACTTGCCGGGCG
AGTCAGGGCATTAGCAATTATTTAGCCTGGTATCAACAGAAACCAGGGAAAGTTCCTAAGCTCCTGATCTATGCT
GCATCCACTTTGCAATCAGGGGTCCCATCTCGGTTCAGTGGCAGTGGATCTGGGACAGATTTCACTCTCACCATC
AGCAGCCTGCAGCCTGAAGATGTTGCAACTTATTACTGTCAAAACCATAACAGTGTCCCTCTCACTTTCGGCGGA
GGGACCAAGGTGGAGATCAAA
LCVR (V_{L}) Amino Acid Sequence
(SEQ ID NO: 17)
DIQMTQSPSSLSASIGDRVTITCRASQGISNYLAWYQQKPGKVPKLLIYAASTLQSGVPSRFSGSGSGTDFTLTI
SSLQPEDVATYYCQNHNSVPLTFGGGTKVEIK
LCDR1:
(SEQ ID NO: 18)
QGISNY
LCDR2:
(SEQ ID NO: 19)
AAS
LCDR3:
(SEQ ID NO: 20)
QNHNSVPLT
69348
HCVR (V_{H}) Nucleotide Sequence
(SEQ ID NO: 21)
CAGGTGCAGCTGGTGGAGTCTGGGGGAGGCGTGGTCCAGCCTGGGAGGTCCCTGAGACTCTCCTGTGCAGCGTCT
GGATTCACCTTCACTACCTATGGCATGCACTGGGTCCGCCAGGCTCCAGGCAAGGGGCTGGAGTGGGTGGCTGTT
ATATGGTATGATGGAAGTAATAAATATTATGGAGACTCCGTGAAGGGCCGATTCACCATCTCCAGAGACAATTCC
AAGAACACACTGTATCTGCAAATGAACAGCCTGAGAGTCGACGACACGGCTGTTTATTACTGTACGAGAACCCAT
GGCTATACCAGGTCGTCGGACGGTTTTGACTACTGGGGCCAGGGAACCCTGGTCACCGTCTCCTCA
HCVR (V_{H}) Amino Acid Sequence
(SEQ ID NO: 22)
QVQLVESGGGVVQPGRSLRLSCAASGFTFTTYGMHWVRQAPGKGLEWVAVIWYDGSNKYYGDSVKGRFTISRDNS
KNTLYLQMNSLRVDDTAVYYCTRTHGYTRSSDGFDYWGQGTLVTVSS
or
(SEQ ID NO: 464)
EVQLVESGGGVVQPGRSLRLSCAASGFTFTTYGMHWVRQAPGKGLEWVAVIWYDGSNKYYGDSVKGRFTISRDNS
KNTLYLQMNSLRVDDTAVYYCTRTHGYTRSSDGFDYWGQGTMVTVSS
HCDR1:
(SEQ ID NO: 23)
GFTFTTYG
HCDR2:
(SEQ ID NO: 24)
IWYDGSNK
HCDR3:
(SEQ ID NO: 25)
TRTHGYTRSSDGEDY
LCVR (V_{L}) Nucleotide Sequence
(SEQ ID NO: 26)
GACATCCAGATGACCCAGTCTCCATCCTCCCTGTCTGCATCTGTAGGAGACAGAGTCACCATCACTTGCCGGGCA
AGTCAGAGCATTAGAAATGTTTTAGGCTGGTTTCAGCAGAAACCAGGGAAAGCCCCTCAGCGCCTGATCTATGCT
GCATCCAGTTTGCAAAGTGGGGTCCCATCAAGGTTCAGCGGCAGTGGATCTGGGACAGAATTCACTCTCACAATC
AGCAGCCTACAGCCTGAAGATTTTGCAACTTATTACTGTCTACAGCATAATTTTTACCCGCTCACTTTCGGCGGA
GGGACCAAGGTGGAGATCAAA
LCVR (V_{L}) Amino Acid Sequence
(SEQ ID NO: 27)
DIQMTQSPSSLSASVGDRVTITCRASQSIRNVLGWFQQKPGKAPQRLIYAASSLQSGVPSRFSGSGSGTEFTLTI
SSLQPEDFATYYCLQHNFYPLTFGGGTKVEIK
LCDR1:
(SEQ ID NO: 28)
QSIRNV
LCDR2:
(SEQ ID NO: 29)
AAS
LCDR3:
(SEQ ID NO: 30)
LQHNFYPLT
69340
HCVR (V_{H}) Nucleotide Sequence
(SEQ ID NO: 31)
GAAGTGCAGCTGGTGGAGTCTGGGGGAGGCTTGGTACAGCCTGGCAGGTCCCTGAGACTCTCCTGTGCAGCCTCT
GGATTCACCTTTGATGATAAAGCCATGCACTGGGTCCGGCAAGTTCCAGGGAAGGGCCTGGAATGGATCTCAGGT
ATTAGTTGGAATAGTGGTACTATAGGCTATGCGGACTCTGTGAAGGGCCGATTCATCATCTCCAGAGACAACGCC
AAGAACTCCCTGTATCTACAAATGAACAGTCTGAGAGCTGAGGACACGGCCTTGTATTACTGCGCAAAAGATGGA
GATACCAGTGGCTGGTACTGGTACGGTTTGGACGTCTGGGGCCAAGGGACCACGGTCACCGTCTCCTCA
HCVR (V_{H}) Amino Acid Sequence
(SEQ ID NO: 32)
EVQLVESGGGLVQPGRSLRLSCAASGFTFDDKAMHWVRQVPGKGLEWISGISWNSGTIGYADSVKGRFIISRDNA
KNSLYLQMNSLRAEDTALYYCAKDGDTSGWYWYGLDVWGQGTTVTVSS
HCDR1:
(SEQ ID NO: 33)
GFTFDDKA
HCDR2:
(SEQ ID NO: 34)
ISWNSGTI
HCDR3:
(SEQ ID NO: 35)
AKDGDTSGWYWYGLDV
LCVR (V_{L}) Nucleotide Sequence
(SEQ ID NO: 36)
GAAATTGTGTTGACACAGTCTCCTGCCACCCTGTCTTTGTCTCCAGGGGAAAGAGCCACCCTCTCCTGCAGGGCC
AGTCAGAGTGTTAGCAGCTACTTAGCCTGGTACCAACAGAAACCTGGCCAGGCTCCCAGGCTCCTCATCCATGAT
GTATCCAACAGGGCCACTGGCATCCCAGCCAGGTTCAGTGGCAGTGGGTCTGGGACAGACTTCACTCTCACCATC
AGCAGTCTAGAGCCTGAAGATTTTGTAGTTTATTACTGTCAGCAGCGTAGCGACTGGCCCATCACCTTCGGCCAA
GGGACACGACTGGAGATTAAA
LCVR (V_{L}) Amino Acid Sequence
(SEQ ID NO: 37)
EIVLTQSPATLSLSPGERATLSCRASQSVSSYLAWYQQKPGQAPRLLIHDVSNRATGIPARFSGSGSGTDFTLTI
SSLEPEDFVVYYCQQRSDWPITFGQGTRLEIK
OR
(SEQ ID NO: 465)
DIVMTQSPATLSLSPGERATLSCRASQSVSSYLAWYQQKPGQAPRLLIHDVSNRATGIPARFSGSGSGTDFTLTI
SSLEPEDFVVYYCQQRSDWPITFGQGTRLEIK
LCDR1:
(SEQ ID NO: 38)
QSVSSY
LCDR2:
(SEQ ID NO: 39)
DVS
LCDR3:
(SEQ ID NO: 40)
QQRSDWPIT
69331
HCVR (V_{H}) Nucleotide Sequence
(SEQ ID NO: 41)
CAGGTGCAGCTGGTGGAGTCTGGGGGAGGCGTGGTCCAGCCTGGGAGGTCCCTGAGACTCTCCTGTATAGCCTCT
GGATTCACCTTCAGTGTCTATGGCATTCACTGGGTCCGCCAGGCTCCAGGCAAGGGGCTGGAGTGGATGGCAGTA
ATATCACATGATGGAAATATTAAACACTATGCAGACTCCGTGAAGGGCCGATTCACCATCTCCAGAGACAATTCC
AAGAACACGCTGTATCTTCAAATTAACAGCCTGAGAACTGAGGACACGGCTGTGTATTACTGTGCGAAAGATACC
TGGAACTCCCTTGATACTTTTGATATCTGGGGCCAAGGGACAATGGTCACCGTCTCTTCA
HCVR (V_{H}) Amino Acid Sequence
(SEQ ID NO: 42)
QVQLVESGGGVVQPGRSLRLSCIASGFTFSVYGIHWVRQAPGKGLEWMAVISHDGNIKHYADSVKGRFTISRDNS
KNTLYLQINSLRTEDTAVYYCAKDTWNSLDTFDIWGQGTMVTVSS
HCDR1:
(SEQ ID NO: 43)
GFTFSVYG
HCDR2:
(SEQ ID NO: 44)
ISHDGNIK
HCDR3:
(SEQ ID NO: 45)
AKDTWNSLDTEDI
LCVR (V_{L}) Nucleotide Sequence
(SEQ ID NO: 46)
GACATCCAGTTGACCCAGTCTCCATCCTCCCTGTCTGCATCTGTAGGAGACAGAGTCACCATCACTTGCTGGGCC
AGTCAGGGCATTAGCAGTTATTTAGCCTGGTATCAGCAAAAACCAGGGAAAGCCCCTAAGCTCCTGATCTATGCT
GCATCCACTTTGCAAAGTGGGGTCCCATCAAGGTTCAGCGGCAGTGGATCTGGGACAGAATTCACTCTCACAATC
AGCAGCCTGCAGCCTGAAGATTTTGCAACTTATTACTGTCAACAGCTTAATAGTTACCCTCTCACTTTCGGCGGA
GGGACCAAGGTGGAGATCAAA
LCVR (V_{L}) Amino Acid Sequence
(SEQ ID NO: 47)
DIQLTQSPSSLSASVGDRVTITCWASQGISSYLAWYQQKPGKAPKLLIYAASTLQSGVPSRFSGSGSGTEFTLTI
SSLQPEDFATYYCQQLNSYPLTFGGGTKVEIK
or
(SEQ ID NO: 466)
DIQMTQSPSSLSASVGDRVTITCWASQGISSYLAWYQQKPGKAPKLLIYAASTLQSGVPSRFSGSGSGTEFTLTI
SSLQPEDFATYYCQQLNSYPLTFGGGTKVEIK
LCDR1:
(SEQ ID NO: 48)
QGISSY
LCDR2:
(SEQ ID NO: 49)
AAS
LCDR3:
(SEQ ID NO: 50)
QQLNSYPLT
69332
HCVR (V_{H}) Nucleotide Sequence
(SEQ ID NO: 51)
CAGGTCACCTTGAGGGAGTCTGGTCCCGCGCTGGTGAAACCCTCACAGACCCTCACACTGACCTGCACCTTCTCT
GGATTCTCACTCAACACTTATGGGATGTTTGTGAGCTGGATCCGTCAGCCTCCAGGGAAGGCCCTAGAGTGGCTT
GCACACATTCATTGGGATGATGATAAATACTACAGCACATCTCTGAAGACCAGGCTCACCATCTCCAAGGACACC
TCCAAAAACCAGGTGGTCCTTACAATGACCAACATGGACCCTGTGGACACAGCCACGTATTATTGTGCACGGGGG
CACAATAATTTGAACTACATCATCCACTGGGGCCAGGGAACCCTGGTCACCGTCTCCTCA
HCVR (V_{H}) Amino Acid Sequence
(SEQ ID NO: 52)
QVTLRESGPALVKPSQTLTLTCTFSGFSLNTYGMFVSWIRQPPGKALEWLAHIHWDDDKYYSTSLKTRLTISKDT
SKNQVVLTMTNMDPVDTATYYCARGHNNLNYIIHWGQGTLVTVSS
or
(SEQ ID NO: 467)
QVQLVESGPALVKPSQTLTLTCTFSGFSLNTYGMFVSWIRQPPGKALEWLAHIHWDDDKYYSTSLKTRLTISKDT
SKNQVVLTMTNMDPVDTATYYCARGHNNLNYIIHWGQGTLVTVSS
HCDR1:
(SEQ ID NO: 53)
GFSLNTYGME
HCDR2:
(SEQ ID NO: 54)
IHWDDDK
HCDR3:
(SEQ ID NO: 55)
ARGHNNLNYIIH
LCVR (V_{L}) Nucleotide Sequence
(SEQ ID NO: 56)
GCCATCCAGATGACCCAGTCTCCATCCTCCCTGTCTGCATCTGTAGGAGACAGAGTCACCATCACTTGCCGGGCA
AGTCAGGGCATTAGAAATGATTTAGGCTGGTATCAGCAGAAACCAGGGAAAGCCCCTAAGCTCCTGATCTATGCT
GCATCCACTTTACAAAGTGGGGTCCCATCAAGGTTCAGCGGCAGTGGATCTGGCACAGATTTCACTCTCACCATC
AGCAGCCTGCAGCCTGAAGATTTTGCAACTTATTACTGTCTACAAGATTACAATTACCCATTCACTTTCGGCCCT
GGGACCAAAGTGGATATCAAA
LCVR (V_{L}) Amino Acid Sequence
(SEQ ID NO: 57)
AIQMTQSPSSLSASVGDRVTITCRASQGIRNDLGWYQQKPGKAPKLLIYAASTLQSGVPSRFSGSGSGTDETLTI
SSLQPEDFATYYCLQDYNYPFTFGPGTKVDIK
or
(SEQ ID NO: 468)
DILMTQSPSSLSASVGDRVTITCRASQGIRNDLGWYQQKPGKAPKLLIYAASTLQSGVPSRFSGSGSGTDFTLTI
SSLQPEDFATYYCLQDYNYPFTFGPGTKVEIK
LCDR1:
(SEQ ID NO: 58)
QGIRND
LCDR2:
(SEQ ID NO: 59)
AAS
LCDR3:
(SEQ ID NO: 60)
LQDYNYPFT
69326
HCVR (V_{H}) Nucleotide Sequence
(SEQ ID NO: 61)
GAGGTGCAGCTGGTGGAGTCTGGGGGAGGCTTGGTACAGCCTGGAGGGTCCCTGAGACTCTCCTGTGCAGTCTCT
GGATTCATCTTCAGTAGTTATGAAATGAACTGGGTCCGCCAGGCTCCAGGGAAGGGGCTGGAGTGGGTTTCATAC
ATTAGTAGTAGTGGTAGTACCATATTCTACGCAGACTCTGTGAAGGGCCGATTCACCATCTCCAGAGACAACGCC
AAGAACTCACTGTATCTGCAAATGAACAGCCTGAGAGCCGAGGACACGGCTGTTTATTACTGTGTGTCTGGAGTG
GTCCTTTTTGATGTCTGGGGCCAAGGGACAATGGTCACCGTCTCTTCA
HCVR (V_{H}) Amino Acid Sequence
(SEQ ID NO: 62)
EVQLVESGGGLVQPGGSLRLSCAVSGFIFSSYEMNWVRQAPGKGLEWVSYISSSGSTIFYADSVKGRFTISRDNA
KNSLYLQMNSLRAEDTAVYYCVSGVVLFDVWGQGTMVTVSS
or
(SEQ ID NO: 492)
QVQLVESGGGLVQPGGSLRLSCAVSGFIFSSYEMNWVRQAPGKGLEWVSYISSSGSTIFYADSVKGRFTISRDNA
KNSLYLQMNSLRAEDTAVYYCVSGVVLFDVWGQGTMVTVSS
HCDR1:
(SEQ ID NO: 63)
GFIFSSYE
HCDR2:
(SEQ ID NO: 64)
ISSSGSTI
HCDR3:
(SEQ ID NO: 65)
VSGVVLFDV
LCVR (V_{L}) Nucleotide Sequence
(SEQ ID NO: 66)
GAAATAGTGATGACGCAGTCTCCAGCCACCCTGTCTGTGTCTCCGGGGGAAAGAGCCACCCTCTCCTGCAGGGCC
AGTCAGAGTGTTAGCAGCAACTTTGCCTGGTACCAACAGAAACCTGGCCAGGCTCCCAGGCTCCTCATCTATAGT
GCATCCTCCAGGGCCACTGGTATCCCAGTCAGGTTCAGTGGCAGTGGGTCTGGGACAGAGTTCACTCTCACCATC
AGCAGCCTGCAGTCTGAAGATTTTGCAGTTTATTACTGTCAGCAGTATAATATCTGGCCTCGGACGTTCGGCCAA
GGGACCAAGGTGGAAATCAAA
LCVR (V_{L}) Amino Acid Sequence
(SEQ ID NO: 67)
EIVMTQSPATLSVSPGERATLSCRASQSVSSNFAWYQQKPGQAPRLLIYSASSRATGIPVRESGSGSGTEFTLTI
SSLQSEDFAVYYCQQYNIWPRTFGQGTKVEIK
or
(SEQ ID NO: 469)
DIVMTQSPATLSVSPGERATLSCRASQSVSSNFAWYQQKPGQAPRLLIYSASSRATGIPVRESGSGSGTEFTLTI
SSLQSEDFAVYYCQQYNIWPRTFGQGTKVEIK
LCDR1:
(SEQ ID NO: 68)
QSVSSN
LCDR2:
(SEQ ID NO: 69)
SAS
LCDR3:
(SEQ ID NO: 70)
QQYNIWPRT
69329
HCVR (V_{H}) Nucleotide Sequence
(SEQ ID NO: 71)
GAGGTGCAGCTGGTGGAGTCTGGGGGAGGCTTGGTCCAGCCTGGGGGGTCCCTGAGACTCTCCTGTGCAGCCTCT
GGATTCACCTTTAGTAACTATTGGATGACCTGGGTCCGCCAGGCTCCAGGGAAGGGGCTGGAGTGGGTGGCCAAC
ATAAAGGAAGATGGAAGTGAGAAAGACTATGTGGACTCTGTGAAGGGCCGATTCACCATCTCCAGAGACAACGCC
AAGAACTCACTGTATCTGCAAATGAACAGCCTGAGAGGCGAGGACACGGCTGTGTATTACTGTGCGAGAGATGGG
GAGCAGCTCGTCGATTACTACTACTACTACGTTATGGACGTCTGGGGCCAAGGGACCACGGTCACCGTCTCCTCA
HCVR (V_{H}) Amino Acid Sequence
(SEQ ID NO: 72)
EVQLVESGGGLVQPGGSLRLSCAASGFTFSNYWMTWVRQAPGKGLEWVANIKEDGSEKDYVDSVKGRFTISRDNA
KNSLYLQMNSLRGEDTAVYYCARDGEQLVDYYYYYVMDVWGQGTTVTVSS
or
(SEQ ID NO: 470)
QVQLVESGGGLVQPGGSLRLSCAASGFTFSNYWMTWVRQAPGKGLEWVANIKEDGSEKDYVDSVKGRFTISRDNA
KNSLYLQMNSLRGEDTAVYYCARDGEQLVDYYYYYVMDVWGQGTTVTVSS
HCDR1:
(SEQ ID NO: 73)
GFTFSNYW
HCDR2:
(SEQ ID NO: 74)
IKEDGSEK
HCDR3:
(SEQ ID NO: 75)
ARDGEQLVDYYYYYVMDV
LCVR (V_{L}) Nucleotide Sequence
(SEQ ID NO: 76)
GACATCCAGATGACCCAGTCTCCATCTTCCGTGTCTGCATCTGTAGGAGACAGAGTCACCATCACTTGTCGGGCG
AGTCAGGGTATTAGCAGCTGGTTAGCCTGGTATCAGCAGAAACCAGGGAAAGCCCCTAAGCTCCTGATCTATGCT
GCATCCAGTTTGCAAAGTGGGGTCCCATCAAGGTTCAGCGGCAGTGGATCTGGGACAGATTTCACTCTCACCATC
AGCAGCCTGCAGCCTGAAGATTTTGCAACTTACTATTGTCAAAAGGCTAACAGTTTCCCGTACACTTTTGGCCAG
GGGACCAAGCTGGAGATCAAA
LCVR (V_{L}) Amino Acid Sequence
(SEQ ID NO: 77)
DIQMTQSPSSVSASVGDRVTITCRASQGISSWLAWYQQKPGKAPKLLIYAASSLQSGVPSRFSGSGSGTDFTLTI
SSLQPEDFATYYCQKANSFPYTFGQGTKLEIK
or
(SEQ ID NO: 471)
DIQMTQSPSSVSASVGDRVTITCRASQGISSWLAWYQQKPGKAPKLLIYAASSLQSGVPSRESGSGSGTDETLTI
SSLQPEDFATYYCQKANSFPYTFGQGTKVEIK
LCDR1:
(SEQ ID NO: 78)
QGISSW
LCDR2:
(SEQ ID NO: 79)
AAS
LCDR3:
(SEQ ID NO: 80)
QKANSFPYT
69323
HCVR (V_{H}) Nucleotide Sequence
(SEQ ID NO: 81)
GAAGTGCAGCTGGTGGAGTCTGGGGGAGGCTTGGTACAGCCTGGCAGGTCCCTGAGACTCTCCTGTGCAGCCTCT
GGATTCACCTTTGATGACTATGCCATGCACTGGGTCCGGCAAGCTCCAGGGAAGGGCCTGGAGTGGGTCTCAGGT
ATTAGTTGGAATAGTGGTTACATAGGCTATGCGGACTCTGTGAAGGGCCGATTCACCATCTCCAGAGACAACGCC
GAGAACTCCCTACATCTGCAAATGAACAGTCTGAGAGCTGAGGACACGGCCTTGTATTACTGTGCAAGAGGGGGA
TCTACTCTGGTTCGGGGAGTTAAGGGAGGCTACTACGGTATGGACGTCTGGGGCCAAGGGACCACGGTCACCGTC
TCCTCA
HCVR (V_{H}) Amino Acid Sequence
(SEQ ID NO: 82)
EVQLVESGGGLVQPGRSLRLSCAASGFTEDDYAMHWVRQAPGKGLEWVSGISWNSGYIGYADSVKGRFTISRDNA
ENSLHLQMNSLRAEDTALYYCARGGSTLVRGVKGGYYGMDVWGQGTTVTVSS
HCDR1:
(SEQ ID NO: 83)
GFTEDDYA
HCDR2:
(SEQ ID NO: 84)
ISWNSGYI
HCDR3:
(SEQ ID NO: 85)
ARGGSTLVRGVKGGYYGMDV
LCVR (V_{L}) Nucleotide Sequence
(SEQ ID NO: 86)
GACATCCAGATGACCCAGTCTCCATCCTCCCTGTCTGCATCTGTAGGAGACAGAGTCACCATCACTTGCCGGGCA
AGTCAGAGCATAAGTAGCTATTTAAATTGGTATCAGCAGAAACCAGGTAAAGCCCCTAAGGTCCTGATCTATGCT
GCATCCAGTTTGCAAAGTGGGGTCCCATCAAGGTTCAGTGGCAGTGGATCTGGGACAGATTTCACTCTCACCATC
AGCAGTCTGCAACCTGAAGATTTTGCAACTTACTACTGTCAACAGAGTTACAGTATTCCGCTCACTTTCGGCGGA
GGGACCAAGGTGGAGATCAAA
LCVR (V_{L}) Amino Acid Sequence
(SEQ ID NO: 87)
DIQMTQSPSSLSASVGDRVTITCRASQSISSYLNWYQQKPGKAPKVLIYAASSLQSGVPSRFSGSGSGTDFTLTI
SSLQPEDFATYYCQQSYSIPLTFGGGTKVEIK
LCDR1:
(SEQ ID NO: 88)
QSISSY
LCDR2:
(SEQ ID NO: 89)
AAS
LCDR3:
(SEQ ID NO: 90)
QQSYSIPLT
69305
HCVR (V_{H}) Nucleotide Sequence
(SEQ ID NO: 91)
CAGGTGCAGCTGGTGGAGTCTGGGGGAGGCGTGGTCCAGCCTGGGAGGTCCCTGAGACTCTCCTGTGCAGCGTCT
GGATTCACCTTCAGTAGCTATGGCATGCACTGGGTCCGCCAGGCTCCAGGCAAGGGGCTGGAGTGGGTGGCAGTT
ATATGGTATGATGGAAGTAATAAATACTATGCAGACTCCGTGAAGGGCCGATTCACCATCTCCAGAGACATTTCC
AAGAACACGCTGTATCTGCAAATGAACAGCCTGAGAGCCGAGGACACGGCCGTATATTACTGTGCGGGTCAACTG
GATCTCTTCTTTGACTACTGGGGCCAGGGAACCCTGGTCACCGTCTCCTCA
HCVR (V_{H}) Amino Acid Sequence
(SEQ ID NO: 92)
QVQLVESGGGVVQPGRSLRLSCAASGFTFSSYGMHWVRQAPGKGLEWVAVIWYDGSNKYYADSVKGRFTISRDIS
KNTLYLQMNSLRAEDTAVYYCAGOLDLFFDYWGQGTLVTVSS
or
(SEQ ID NO: 472)
EVQLVESGGGVVQPGRSLRLSCAASGFTFSSYGMHWVRQAPGKGLEWVAVIWYDGSNKYYADSVKGRFTISRDIS
KNTLYLQMNSLRAEDTAVYYCAGQLDLFFDYWGQGTLVTVSS
HCDR1:
(SEQ ID NO: 93)
GFTFSSYG
HCDR2:
(SEQ ID NO: 94)
IWYDGSNK
HCDR3:
(SEQ ID NO: 95)
AGQLDLFFDY
LCVR (V_{L}) Nucleotide Sequence
(SEQ ID NO: 96)
GACATCCAGATGACCCAGTCTCCATCCTCCCTGTCTGCATCTGTAGGAGACAGAGTCACCATCACTTGCCGGGCA
AGTCAGAGCATTGACAGGTATTTAAATTGGTATCGGCAGAAACCAGGGAAAGCCCCTAAGCTCCTGATCTATACT
ACATCCAGTTTGCAAAGTGGGGTCCCATCAAGGTTCAGTGGCAGTGGATCTGGGACAGATTTCACTCTCACCCTC
AGCAGTCTGCAGCCTGAAGATTTTGCAACTTACTACTGTCAGCAGAGTTACAGTCCCCCGCTCACTTTCGGCGGA
GGGACCAAGGTGGAGATCAAA
LCVR (V_{L}) Amino Acid Sequence
(SEQ ID NO: 97)
DIQMTQSPSSLSASVGDRVTITCRASQSIDRYLNWYRQKPGKAPKLLIYTTSSLQSGVPSRFSGSGSGTDFTLTL
SSLQPEDFATYYCQQSYSPPLTFGGGTKVEIK
LCDR1:
(SEQ ID NO: 98)
QSIDRY
LCDR2:
(SEQ ID NO: 99)
TTS
LCDR3:
(SEQ ID NO: 100)
QQSYSPPLT
69307
HCVR (V_{H}) Nucleotide Sequence
(SEQ ID NO: 101)
GAGGTGCAGCTGGTGGAGTCTGGGGGAGGCTTGGTCCAGCCTGGGGGGTCCCTGAGACTCTCCTGTACAGCCTCT
GGATTCACCTTTAGTAACTATTGGATGACCTGGGTCCGCCAGGCTCCAGGGAAGGGGCTGGAGTGGGTGGCCAAC
ATAAAGGAAGATGGAAGTGAGAAAGAGTATGTGGACTCTGTGAAGGGCCGGTTCACCATCTCCAGAGACAACGCC
AAGAATTCACTGTATCTGCAAATGAACAGCCTGAGAGGCGAGGACACGGCTGTATATTACTGTGCGAGAGATGGG
GAGCAGCTCGTCGATTACTATTACTACTACGTTATGGACGTCTGGGGCCAAGGGACCACGGTCACCGTCTCCTCA
HCVR (V_{H}) Amino Acid Sequence
(SEQ ID NO: 102)
EVQLVESGGGLVQPGGSLRLSCTASGFTFSNYWMTWVRQAPGKGLEWVANIKEDGSEKEYVDSVKGRFTISRDNA
KNSLYLQMNSLRGEDTAVYYCARDGEQLVDYYYYYVMDVWGQGTTVTVSS
HCDR1:
(SEQ ID NO: 103)
GFTFSNYW
HCDR2:
(SEQ ID NO: 104)
IKEDGSEK
HCDR3:
(SEQ ID NO: 105)
ARDGEQLVDYYYYYVMDV
LCVR (V_{L}) Nucleotide Sequence
(SEQ ID NO: 106)
GACATCCAGATGACCCAGTCTCCATCTTCCGTGTCTGCATCTGTTGGAGACAGAGTCACCATCACTTGTCGGGCG
AGTCAGGGTATTAGCAGCTGGTTAGCCTGGTATCAGCAGAAACCAGGGAAAGCCCCTAAGCTCCTGATCTATGCT
GCATCCAGTTTGCAAAGTGGGGTCCCATCAAGGTTCAGCGGCAGTGGATCTGGGACAGATTTCACTCTCACCATC
AGCAGCCTGCAGCCTGAAGATTTTGCAACTTACTATTGTCAAAAGGCTGACAGTCTCCCGTACGCTTTTGGCCAG
GGGACCAAGCTGGAGATCAAA
LCVR (V_{L}) Amino Acid Sequence
(SEQ ID NO: 107)
DIQMTQSPSSVSASVGDRVTITCRASQGISSWLAWYQQKPGKAPKLLIYAASSLQSGVPSRFSGSGSGTDETLTI
SSLQPEDFATYYCQKADSLPYAFGQGTKLEIK
LCDR1:
(SEQ ID NO: 108)
QGISSW
LCDR2:
(SEQ ID NO: 109)
AAS
LCDR3:
(SEQ ID NO: 110)
QKADSLPYA
12795B
HCVR (V_{H}) Nucleotide Sequence
(SEQ ID NO: 111)
GAGGTGCAGCTGGTGGAGTCTGGGGGAGGCTTGGTTCAGCCTGGGGGGTCCCTGAGACTCTCCTGTGCAACCTCT
GGATTCACCTTTACCAGCTATGACATGAAGTGGGTCCGCCAGGCTCCAGGGCTGGGCCTGGAGTGGGTCTCAGCT
ATTAGTGGTAGTGGTGGTAACACATACTACGCAGACTCCGTGAAGGGCCGGTTCACCATCTCCAGAGACAATTCC
AGGAACACGCTGTATCTGCAAATGAACAGCCTGAGAGCCGAGGACACGGCCGTATATTACTGTACGAGGTCCCAT
GACTTCGGTGCCTTCGACTACTTTGACTACTGGGGCCAGGGAACCCTGGTCACCGTCTCCTCA
HCVR (V_{H}) Amino Acid Sequence
(SEQ ID NO: 112)
EVQLVESGGGLVQPGGSLRLSCATSGFTFTSYDMKWVRQAPGLGLEWVSAISGSGGNTYYADSVKGRFTISRDNS
RNTLYLQMNSLRAEDTAVYYCTRSHDFGAFDYFDYWGQGTLVTVSS
or
(SEQ ID NO: 473)
EVQLVQSGGGLVQPGGSLRLSCATSGFTFTSYDMKWVRQAPGLGLEWVSAISGSGGNTYYADSVKGRFTISRDNS
RNTLYLQMNSLRAEDTAVYYCTRSHDFGAFDYFDYWGQGTMVTVSS
HCDR1:
(SEQ ID NO: 113)
GFTFTSYD
HCDR2:
(SEQ ID NO: 114)
ISGSGGNT
HCDR3:
(SEQ ID NO: 115)
TRSHDFGAFDYEDY
LCVR (V_{L}) Nucleotide Sequence
(SEQ ID NO: 116)
GACATCCAGATGACCCAGTCTCCATCCTCCCTGTCTGCATCTGTGGGAGACAGAGTCACCATCACTTGCCGGGCA
AGTCAGGGCATTAGAGATCATTTTGGCTGGTATCAGCAGAAACCAGGGAAAGCCCCTAAGCGCCTGATCTATGCT
GCATCCAGTTTGCACAGTGGGGTCCCATCAAGGTTCAGCGGCAGTGGATCTGGGACAGAATTCACTCTCACAATC
AGCAGCTTGCAGCCTGAAGATTTTGCAACCTATTACTGTCTACAGTATGATACTTACCCGCTCACTTTCGGCGGA
GGGACCAAGGTGGAGATCAAA
LCVR (V_{L}) Amino Acid Sequence
(SEQ ID NO: 117)
DIQMTQSPSSLSASVGDRVTITCRASQGIRDHFGWYQQKPGKAPKRLIYAASSLHSGVPSRFSGSGSGTEFTLTI
SSLQPEDFATYYCLQYDTYPLTFGGGTKVEIK
or
(SEQ ID NO: 474)
DIQLTQSPSSLSASVGDRVTITCRASQGIRDHFGWYQQKPGKAPKRLIYAASSLHSGVPSRFSGSGSGTEFTLTI
SSLQPEDFATYYCLQYDTYPLTFGGGTKVEIK
LCDR1:
(SEQ ID NO: 118)
QGIRDI
LCDR2:
(SEQ ID NO: 119)
AAS
LCDR3:
(SEQ ID NO: 120)
LQYDTYPLT
12798B
HCVR (V_{H}) Nucleotide Sequence
(SEQ ID NO: 121)
GAAGTGCAGCTGGTGGAGTCTGGGGGAGACTTGGTACAGCCTGGCAGGTCCCTGAGACTCTCCTGTGCAGCCTCT
GGATTCACCTTTGATGATTATGCCATGCACTGGGTCCGGCAAGCTCCAGGGAAGGGCCTGGAGTGGGTCTCAGGT
ATTAGTTGGAATAGTGCTACCAGAGTCTATGCGGACTCTGTGAAGGGCCGATTCACCATCTCCAGAGACAACGCC
AAGAATTTCCTGTATCTGCAAATGAACAGTCTGAGATCTGAGGACACGGCCTTGTATCACTGTGCAAAAGATATG
GATATCTCGCTAGGGTACTACGGTTTGGACGTCTGGGGCCAAGGGACCACGGTCACCGTCTCCTCA
HCVR (V_{H}) Amino Acid Sequence
(SEQ ID NO: 122)
EVQLVESGGDLVQPGRSLRLSCAASGFTFDDYAMHWVRQAPGKGLEWVSGISWNSATRVYADSVKGRFTISRDNA
KNFLYLQMNSLRSEDTALYHCAKDMDISLGYYGLDVWGQGTTVTVSS
HCDR1:
(SEQ ID NO: 123)
GFTFDDYA
HCDR2:
(SEQ ID NO: 124)
ISWNSATR
HCDR3:
(SEQ ID NO: 125)
AKDMDISLGYYGLDV
LCVR (V_{L}) Nucleotide Sequence
(SEQ ID NO: 126)
GAAATAGTGATGACGCAGTCTCCAGCCACCCTGTCTGTGTCTCCAGGGGAAAGAGCCACCCTCTCCTGCAGGGCC
AGTCAGACTGTTAGCAGCAACTTAGCCTGGTATCAGCAGAAACCTGGCCAGGCTCCCAGGCTCCTCATCTATGGT
TCATCCTCCAGGGCCACTGGTATCCCAGCCAGGTTCAGTGGCAGTGGGTCTGGGACAGAGTTCACTCTCACCATC
AGCAGCCTGCAGTCTGAAGATTTTGCAGTTTATTACTGTCAGCAGTATAATAACTGGCCTCCCTACACTTTTGGC
CAGGGGACCAAGCTGGAGATCAAA
LCVR (V_{L}) Amino Acid Sequence
(SEQ ID NO: 127)
EIVMTQSPATLSVSPGERATLSCRASQTVSSNLAWYQQKPGQAPRLLIYGSSSRATGIPARFSGSGSGTEFTLTI
SSLQSEDFAVYYCQQYNNWPPYTFGQGTKLEIK
LCDR1:
(SEQ ID NO: 128)
QTVSSN
LCDR2:
(SEQ ID NO: 129)
GSS
LCDR3:
(SEQ ID NO: 130)
QQYNNWPPYT
12799B
HCVR (V_{H}) Nucleotide Sequence
(SEQ ID NO: 131)
CAGATCACCTTGAAGGAGTCTGGTCCTACGCTGGTGAAACCCACACAGACCCTCACGCTGACCTGCACCTTCTCT
GGGTTCTCACTCAGCACTAGTGGAGTGGGTGTGGTCTGGATCCGTCAGCCCCCCGGAAAGGCCCTGGAGTGGCTT
GCACTCATTTATTGGAATGATCATAAGCGGTACAGCCCATCTCTGGGGAGCAGGCTCACCATCACCAAGGACACC
TCCAAAAACCAGGTGGTCCTTACAATGACCAACATGGACCCTGTGGACACAGCCACATATTACTGTGCACACTAC
AGTGGGAGCTATTCCTACTACTACTATGGTTTGGACGTCTGGGGCCAAGGGACCACGGTCACCGTCTCCTCA
HCVR (V_{H}) Amino Acid Sequence
(SEQ ID NO: 132)
QITLKESGPTLVKPTQTLTLTCTFSGFSLSTSGVGVVWIRQPPGKALEWLALIYWNDHKRYSPSLGSRLTITKDT
SKNQVVLTMTNMDPVDTATYYCAHYSGSYSYYYYGLDVWGQGTTVTVSS
HCDR1:
(SEQ ID NO: 133)
GFSLSTSGVG
HCDR2:
(SEQ ID NO: 134)
IYWNDHK
HCDR3:
(SEQ ID NO: 135)
AHYSGSYSYYYYGLDV
LCVR (V_{L}) Nucleotide Sequence
(SEQ ID NO: 136)
GACATCCAGATGACCCAGTCTCCATCTTCCGTGTCTGCATCTGTAGGAGACAGAGTCACCATCACTTGTCGGGCG
AGTCAGGGTATTGCCAGCTGGTTAGCCTGGTATCAGCAGAAACCAGGGAAAGCCCCTGAGCTCCTGATCTATGCT
GCATCCAGTTTGCAAGGTGGGGTCCCATCAAGGTTCAGCGGCAGTGGATCTGGGACAGATTTCACTCTCACCATC
AGCAGCCTGCAGCCTGAAGATTTTGCAATTTACTATTGTCAACAGGCTAACTATTTCCCGTGGACGTTCGGCCAA
GGGACCAAGGTGGAAATCAAA
LCVR (V_{L}) Amino Acid Sequence
(SEQ ID NO: 137)
DIQMTQSPSSVSASVGDRVTITCRASQGIASWLAWYQQKPGKAPELLIYAASSLQGGVPSRFSGSGSGTDFTLTI
SSLQPEDFAIYYCQQANYFPWTFGQGTKVEIK
LCDR1:
(SEQ ID NO: 138)
QGIASW
LCDR2:
(SEQ ID NO: 139)
AAS
LCDR3:
(SEQ ID NO: 140)
QQANYFPWT
12801B
HCVR (V_{H}) Nucleotide Sequence
(SEQ ID NO: 141)
GAGGTGCAGCTGTTGGAGTCTGGGGGAGCCTTGGTACAGCCTGGGGGGTCCCTGAGACTCTCCTGTGCAGCCTCT
GGATTCACCTTTACCTCCTATGCCATGCACTGGGTCCGCCAGGCTCCAGGGAAGGGTCTGGAGTGGGTCTCATCT
ATTAGAGGTAGTGGTGGTGGCACATACTCCGCAGACTCCGTGAAGGGCCGGTTCACCATCTCCAGAGACAATTCC
AGGGACACTCTATATCTGCAAATGAACAGTGTGAGAGCCGAGGACACGGCCGTTTATTACTGTGCGAGGTCCCAT
GACTACGGTGCCTTCGACTTCTTTGACTACTGGGGCCAGGGAACCCTGGTCACCGTCTCCTCA
HCVR (V_{H}) Amino Acid Sequence
(SEQ ID NO: 142)
EVQLLESGGALVQPGGSLRLSCAASGFTFTSYAMHWVRQAPGKGLEWVSSIRGSGGGTYSADSVKGRFTISRDNS
RDTLYLQMNSVRAEDTAVYYCARSHDYGAFDFFDYWGQGTLVTVSS
or
(SEQ ID NO: 475)
EVQLLESGGALVQPGGSLRLSCAASGFTFTSYAMHWVRQAPGKGLEWVSSIRGSGGGTYSADSVKGRFTISRDNS
RDTLYLQMNSVRAEDTAVYYCARSHDYGAFDFFDYWGQGTTVTVSS
HCDR1:
(SEQ ID NO: 143)
GFTFTSYA
HCDR2:
(SEQ ID NO: 144)
IRGSGGGT
HCDR3:
(SEQ ID NO: 145)
ARSHDYGAFDFFDY
LCVR (V_{L}) Nucleotide Sequence
(SEQ ID NO: 146)
GACATCCAGATGACCCAGTCTCCATCCTCCCTGTCTGCATCTGTAGGAGACAGAGTCACCATCACTTGCCGGGCA
AGTCAGGGCATTAGAACTGATTTAGGCTGGTATCAGCAGAAACCAGGGAAAGCCCCTAAGCGCCTGATCTATGCT
GCATCCAGTTTGCAAAGTGGGGTCCCATCAAGGTTCAGCGGCAGTGGATCTGGGACAGAATTCACTCTCACAATC
AGCAGCCTGCGGCCTGAAGATTTTGCAACTTTTTACTGTCTACAGTATAATAGTTACCCGCTCACTTTCGGCGGA
GGGACCAAGGTGGAGATCAAA
LCVR (V_{L}) Amino Acid Sequence
(SEQ ID NO: 147)
DIQMTQSPSSLSASVGDRVTITCRASQGIRTDLGWYQQKPGKAPKRLIYAASSLQSGVPSRFSGSGSGTEFTLTI
SSLRPEDFATFYCLQYNSYPLTFGGGTKVEIK
or
(SEQ ID NO: 476)
DIQMTQSPSSLSASVGDRVTITCRASQGIRTDLGWYQQKPGKAPKRLIYAASSLQSGVPSRESGSGSGTEFTLTI
SSLRPEDFATFYCLQYNSYPLTFGGGTKVDIK
LCDR1:
(SEQ ID NO: 148)
QGIRTD
LCDR2:
(SEQ ID NO: 149)
AAS
LCDR3:
(SEQ ID NO: 150)
LQYNSYPLT
12802B
HCVR (V_{H}) Nucleotide Sequence
(SEQ ID NO: 151)
CAGGTGCAGCTGGTGGAGTCTGGGGGAGGCTTGGTCAAGCCTGGAGGGTCCCTGAGACTCTCCTGTGCAGCCTCT
GGATTCACCTTCAGTGACTACTTCATGAGCTGGATCCGCCAGGCTCCAGGGAAGGGGCTGGAGTGGGTTTCATAC
ATTAGTAGTACTGGTAGTACCATAAATTATGCAGACTCTGTGAAGGGCCGATTCACCATCTCCAGGGACAATGTC
AAGAATTCACTGTATCTGCAAATGACCAGCCTGAGAGTCGAGGACACGGCCGTGTATTACTGTACGAGAGATAAC
TGGAACTATGAATACTGGGGCCAGGGAACCCTGGTCACCGTCTCCTCA
HCVR (V_{H}) Amino Acid Sequence
(SEQ ID NO: 152)
QVQLVESGGGLVKPGGSLRLSCAASGFTFSDYFMSWIRQAPGKGLEWVSYISSTGSTINYADSVKGRFTISRDNV
KNSLYLQMTSLRVEDTAVYYCTRDNWNYEYWGQGTLVTVSS
HCDR1:
(SEQ ID NO: 153)
GFTFSDYF
HCDR2:
(SEQ ID NO: 154)
ISSTGSTI
HCDR3:
(SEQ ID NO: 155)
TRDNWNYEY
LCVR (V_{L}) Nucleotide Sequence
(SEQ ID NO: 156)
GAAATAGTGATGACGCAGTCTCCAGCCACCCTGTCTGTGTCTCCAGGGGAAAGAGCCACCCTCTCCTGCAGGGCC
AGTCAGAGTGTTAGCATCAACTTAGCCTGGTACCAGCAGAAACCTGGCCAGGCTCCCAGGCTCCTCATCTTTGTT
GCATCCACCAGGGCCACTGGTATCCCAGCCAGGTTCAGTGGCAGTGGGTCTGGGACAGAGTTCACTCTCACCATC
AGCAGCCTGCAGTCTGAAGATTTTGCAACTTATTACTGTCAGCAGTATGATATCTGGCCGTACACTTTTGGCCAG
GGGACCAAGCTGGAGATCAAA
LCVR (V_{L}) Amino Acid Sequence
(SEQ ID NO: 157)
EIVMTQSPATLSVSPGERATLSCRASQSVSINLAWYQQKPGQAPRLLIFVASTRATGIPARFSGSGSGTEFTLTI
SSLQSEDFATYYCQQYDIWPYTFGQGTKLEIK
LCDR1:
(SEQ ID NO: 158)
QSVSIN
LCDR2:
(SEQ ID NO: 159)
VAS
LCDR3:
(SEQ ID NO: 160)
QQYDIWPYT
12808B
HCVR (V_{H}) Nucleotide Sequence
(SEQ ID NO: 161)
CAGCTGCAGCTGCAGGAGTCGGGCCCAGGACTGGTGAAGCCTTCGGAGACCCTGTCCCTCACCTGCACTGTGTCT
GGTGAATCCATCAGCAGTAATACTTACTACTGGGGCTGGATCCGCCAGCCCCCAGGGAAGGGGCTGGAATGGATT
GGGAGTATCGATTATAGTGGGACCACCAATTATAACCCGTCCCTCAAGAGTCGAGTCACCATATCCGTAGACACG
TCCAGGAATCACTTCTCCCTGAGGCTGAGGTCTGTGACCGCCGCAGACACGGCTGTGTATTACTGTGCGAGAGAG
TGGGGAAACTACGGCTACTATTACGGTATGGACGTTTGGGGCCAAGGGACCACGGTCACCGTCTCCTCA
HCVR (V_{H}) Amino Acid Sequence
(SEQ ID NO: 162)
QLQLQESGPGLVKPSETLSLTCTVSGESISSNTYYWGWIRQPPGKGLEWIGSIDYSGTTNYNPSLKSRVTISVDT
SRNHFSLRLRSVTAADTAVYYCAREWGNYGYYYGMDVWGQGTTVTVSS
or
(SEQ ID NO: 477)
QVQLVESGPGLVKPSETLSLTCTVSGESISSNTYYWGWIRQPPGKGLEWIGSIDYSGTTNYNPSLKSRVTISVDT
SRNHFSLRLRSVTAADTAVYYCAREWGNYGYYYGMDVWGQGTTVTVSS
HCDR1:
(SEQ ID NO: 163)
GESISSNTYY
HCDR2:
(SEQ ID NO: 164)
IDYSGTT
HCDR3:
(SEQ ID NO: 165)
AREWGNYGYYYGMDV
LCVR (V_{L}) Nucleotide Sequence
(SEQ ID NO: 166)
GACATCCAGATGACCCAGTCTCCATCCTCCCTGTCTGCATCTGTAGGAGACAGAGTCACCATCAATTGCCGGGCA
AGTCAGGGCATTAGAAATGATTTAGGCTGGTATCAGCAGAAACCAGGGAAAGCCCCTAAGCGCCTGATCTATGCT
GCATCCAGTTTGCAAAGTGGGGTCCCATTAAGGTTCAGTGGCAGTGGATCTGGGACAGAATTCACTCTCACAATC
AACAACCTGCAGCCTGAAGATTTTGCAACTTATTACTGTCTATCGCATAATAGTTACCCGTGGACGTTCGGCCAA
GGGACCAAGGTGGAAATCAAA
LCVR (V_{L}) Amino Acid Sequence
(SEQ ID NO: 167)
DIQMTQSPSSLSASVGDRVTINCRASQGIRNDLGWYQQKPGKAPKRLIYAASSLQSGVPLRFSGSGSGTEFTLTI
NNLQPEDFATYYCLSHNSYPWTFGQGTKVEIK
LCDR1:
(SEQ ID NO: 168)
QGIRND
LCDR2:
(SEQ ID NO: 169)
AAS
LCDR3:
(SEQ ID NO: 170)
LSHNSYPWT
12812B
HCVR (V_{H}) Nucleotide Sequence
(SEQ ID NO: 171)
CAGGTGCAGCTGGTGCAGTCTGGGGCTGAGGTGAAGAAGCCTGGGTCCTCGGTGAGGGTCTCCTGCAAGGCTTCT
AGAGGCACCTTCAGCAGCTATGCTATCAGCTGGGTGCGACAGGCCCCTGGACAAGGCCTTGAGTGGATGGGAGGG
ATCATCCCCATCTTTGGTACAGCAAACTACGCACAGAAGTTCCTGGCCAGAGTCACGATTACCGCGGACGAATCC
ACGAGCACAGCCTACATGGAGCTGAGCAGCCTGAGATCTGAGGACACGGCCGTGTATTACTGTGCGAGAGAGAAG
GGGTGGAACTACTTTGACTACTGGGGCCAGGGAACCCTGGTCACCGTCTCCTCA
HCVR (V_{H}) Amino Acid Sequence
(SEQ ID NO: 172)
QVQLVQSGAEVKKPGSSVRVSCKASRGTFSSYAISWVRQAPGQGLEWMGGIIPIFGTANYAQKFLARVTITADES
TSTAYMELSSLRSEDTAVYYCAREKGWNYFDYWGQGTLVTVSS
HCDR1:
(SEQ ID NO: 173)
RGTFSSYA
HCDR2:
(SEQ ID NO: 174)
ITPIFGTA
HCDR3:
(SEQ ID NO: 175)
AREKGWNYFDY
LCVR (V_{L}) Nucleotide Sequence
(SEQ ID NO: 176)
GACATCCAGATGACCCAGTCTCCACCTTCCGTGTCTGCATCTGTAGGAGACAGAGTCACCATCACTTGTCGGGCG
AGTCAGGGTATTAGCAGCTGGTTAGCCTGGTATCAGCAGAAACCAGGGAAAGCCCCTAAACTCCTGATCTATGCT
GCATCCAGTTTGCAAAGTGGGGTCCCATCAAGGTTCAGCGGCAGTGGATCTGGGACAGATTTCACTCTCACCATC
AGCAGCCTGCAGCCTGAAGATTTTGCAACTTACTATTGTCAACAGGCTAACAGTTTCCCTCGGACGTTCGGCCAA
GGGACCAAGGTGGAAATCAAA
LCVR (V_{L}) Amino Acid Sequence
(SEQ ID NO: 177)
DIQMTQSPPSVSASVGDRVTITCRASQGISSWLAWYQQKPGKAPKLLIYAASSLQSGVPSRESGSGSGTDFTLTI
SSLQPEDFATYYCQQANSFPRTFGQGTKVEIK
LCDR1:
(SEQ ID NO: 178)
QGISSW
LCDR2:
(SEQ ID NO: 179)
AAS
LCDR3:
(SEQ ID NO: 180)
QQANSFPRT
12816B
HCVR (V_{H}) Nucleotide Sequence
(SEQ ID NO: 181)
CAGGTGCAGCTGGTGGAGTCTGGGGGAGGCTTGGTCAAGCCTGGAGGGTCCCTGAGACTCTCCTGTGCAGCCTCT
GGATTCACCTTCAGTGACTACTACATGAACTGGATCCGCCAGGCTCCAGGGAAGGGGCTGGAGTGGGTTTCATAC
ATTAGTAGTAGTGGGACTACCATATACTACGCAGACTCTGTGAAGGGCCGATTCACCATCTCCAGGGACAACGCC
AAGAAATCACTGTATCTGGAGATGAACAGCCTCAGAGCCGAGGACACGGCCGTGTACTACTGTGCGAGAGAGGGG
TACGGTAATGACTACTATTACTACGGTATAGACGTCTGGGGCCAAGGGACCACGGTCACCGTCTCCTCA
HCVR (V_{H}) Amino Acid Sequence
(SEQ ID NO: 182)
QVQLVESGGGLVKPGGSLRLSCAASGFTFSDYYMNWIRQAPGKGLEWVSYISSSGTTIYYADSVKGRFTISRDNA
KKSLYLEMNSLRAEDTAVYYCAREGYGNDYYYYGIDVWGQGTTVTVSS
or
(SEQ ID NO: 478)
EVQLVESGGGLVKPGGSLRLSCAASGFTFSDYYMNWIRQAPGKGLEWVSYISSSGTTIYYADSVKGRFTISRDNA
KKSLYLEMNSLRAEDTAVYYCAREGYGNDYYYYGIDVWGQGTTVTVSS
HCDR1:
(SEQ ID NO: 183)
GFTFSDYY
HCDR2:
(SEQ ID NO: 184)
ISSSGTTI
HCDR3:
(SEQ ID NO: 185)
AREGYGNDYYYYGIDV
LCVR (V_{L}) Nucleotide Sequence
(SEQ ID NO: 186)
GATATTGTGATGACTCAGTCTCCACTCTCCCTGCCCGTCACCCCTGGAGAGCCGGCCTCCATCTCCTGCAGGTCT
AGTCAGAGCCTCCTGCATGGTAATGGATACAACTATTTGACTTGGTACCTGCAGAAGCCAGGGCAGTCTCCACAG
CTCCTGATCTATTTGGGTTCTAATCGGGCCTCCGGGGTCCCTGACAGGTTCAGTGGCAGTGGATCAGGCACAGAT
TTTACACTGAAAATAAGCAGAGTGGAGGCTGAGGATGTTGGGGTTTATTACTGCATGCAAGCTCTACAAACTCCG
TACACTTTTGGCCAGGGGACCAAGCTGGAGATCAAA
LCVR (V_{L}) Amino Acid Sequence
(SEQ ID NO: 187)
DIVMTQSPLSLPVTPGEPASISCRSSQSLLHGNGYNYLTWYLQKPGQSPQLLIYLGSNRASGVPDRFSGSGSGTD
FTLKISRVEAEDVGVYYCMQALQTPYTFGQGTKLEIK
or
(SEQ ID NO: 479)
DIQLTQSPLSLPVTPGEPASISCRSSQSLLHGNGYNYLTWYLQKPGQSPQLLIYLGSNRASGVPDRFSGSGSGTD
FTLKISRVEAEDVGVYYCMQALQTPYTFGQGTKVEIK
LCDR1:
(SEQ ID NO: 188)
QSLLHGNGYNY
LCDR2:
(SEQ ID NO: 189)
LGS
LCDR3:
(SEQ ID NO: 190)
MQALOTPYT
12833B
HCVR (V_{H}) Nucleotide Sequence
(SEQ ID NO: 191)
CAGGTGCAGCTGGTGGAGTCTGGGGGAGGCGTGGTCCAGCCTGGGAGGTCCCTGAGACTCTCCTGTGCAGCCTCT
GGATTCACCTTCAGTAGCTTTGGCATGCACTGGGTCCGCCAGGCTCCAGGCAAGGGGCTGGAGTGGGTGATATTT
ATATCATATGATGGAAGTGATAAATACTATGCAGACTCCGTGAAGGGCCGATTCGCCATCTCCAGAGACAGTTCC
AAGAACACGCTATATCTGCAAATGAACAGCCTGAGAGCTGAGGACACGGCTGTGTATTACTGTGCGAAAGAAAAC
GGTATTTTGACTGATTCCTACGGTATGGACGTCTGGGGCCAAGGGACCACGGTCACCGTCTCCTCA
HCVR (V_{H}) Amino Acid Sequence
(SEQ ID NO: 192)
QVQLVESGGGVVQPGRSLRLSCAASGFTFSSFGMHWVRQAPGKGLEWVIFISYDGSDKYYADSVKGRFAISRDSS
KNTLYLQMNSLRAEDTAVYYCAKENGILTDSYGMDVWGQGTTVTVSS
or
(SEQ ID NO: 480)
EVQLVESGGGVVQPGRSLRLSCAASGFTFSSFGMHWVRQAPGKGLEWVIFISYDGSDKYYADSVKGRFAISRDSS
KNTLYLQMNSLRAEDTAVYYCAKENGILTDSYGMDVWGQGTTVTVSS
HCDR1:
(SEQ ID NO: 193)
GFTFSSFG
HCDR2:
(SEQ ID NO: 194)
ISYDGSDK
HCDR3:
(SEQ ID NO: 195)
AKENGILTDSYGMDV
LCVR (V_{L}) Nucleotide Sequence
(SEQ ID NO: 196)
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAA
LCVR (V_{L}) Amino Acid Sequence
(SEQ ID NO: 197)
DIQMTQSPSSLSASVGDRVTITCRASQSISSYLNWYQQKPGKAPKLLIYAASSLQSGVPSRFSGSGSGTDETLTI
SSLQPEDFATYYCQQSYSTPPITFGQGTRLEIK
LCDR1:
(SEQ ID NO: 198)
QSISSY
LCDR2:
(SEQ ID NO: 199)
AAS
LCDR3:
(SEQ ID NO: 200)
QQSYSTPPIT
12834B
HCVR (V_{H}) Nucleotide Sequence
(SEQ ID NO: 201)
CAGGTTCAGCTGGTGCAGTCTGGAGCTGAGGTGAAGAAGCCTGGGGCCTCTGTGAAGGTCTCCTGCAAGGCTTCT
GGTTACACCTTTACCAGCTATGGTATCAGCTGGGTGCGACAGGCCCCTGGACAAGGGCTTGAGTGGATGGGATGG
ATCAGTGTTTACCATGGTAACACAAACTATGCACAGAAGTTCCAGGGCAGAGTCACCATGACCACAGACACATCC
ACGAGCACAGCCTACATGGAGCTGAGGAGCCTGAGATCTGACGACACGGCCGTGTATTACTGTGCGAGAGAGGGG
TATTACGATTTTTGGAGTGGTTATTACCCTTTTGACTACTGGGGCCAGGGAACCCTGGTCACCGTCTCCTCA
HCVR (V_{H}) Amino Acid Sequence
(SEQ ID NO: 202)
QVQLVQSGAEVKKPGASVKVSCKASGYTFTSYGISWVRQAPGQGLEWMGWISVYHGNTNYAQKFQGRVTMTTDTS
TSTAYMELRSLRSDDTAVYYCAREGYYDFWSGYYPFDYWGQGTLVTVSS
or
(SEQ ID NO: 481)
EVQLVESGAEVKKPGASVKVSCKASGYTFTSYGISWVRQAPGQGLEWMGWISVYHGNTNYAQKFQGRVTMTTDTS
TSTAYMELRSLRSDDTAVYYCAREGYYDFWSGYYPFDYWGQGTTVTVSS
HCDR1:
(SEQ ID NO: 203)
GYTFTSYG
HCDR2:
(SEQ ID NO: 204)
ISVYHGNT
HCDR3:
(SEQ ID NO: 205)
AREGYYDFWSGYYPFDY
LCVR (V_{L}) Nucleotide Sequence
(SEQ ID NO: 206)
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAA
LCVR (V_{L}) Amino Acid Sequence
(SEQ ID NO: 207)
DIQMTQSPSSLSASVGDRVTITCRASQSISSYLNWYQQKPGKAPKLLIYAASSLQSGVPSRFSGSGSGTDETLTI
SSLQPEDFATYYCQQSYSTPPITFGQGTRLEIK
LCDR1:
(SEQ ID NO: 208)
QSISSY
LCDR2:
(SEQ ID NO: 209)
AAS
LCDR3:
(SEQ ID NO: 210)
QQSYSTPPIT
12835B
HCVR (V_{H}) Nucleotide Sequence
(SEQ ID NO: 211)
GAGGTGCAGCTGGTGGAGTCTGGGGGAGGCTTGATACAACCTGGAGGGTCCCTGAGACTCTCCTGTGAAGCCTCT
GGATTCACCTTCAGAAATTATGAAATGAATTGGGTCCGCCAGGCTCCAGGGAAGGGGCTGGAGTGGGTTTCATAT
ATTAGTAGTAGTGGTAATATGAAAGACTACGCAGAGTCTGTGAAGGGCCGATTCACCATCTCCAGAGACAATGTC
AAGAATTCACTGCAGCTGCAAATGAACAGCCTGAGAGTCGAGGACACGGCTGTTTATTACTGTGCGAGAGACGAG
TTTCCTTACGGAATGGACGTCTGGGGCCAAGGGACCACGGTCACCGTCTCCTCA
HCVR (V_{A}) Amino Acid Sequence
(SEQ ID NO: 212)
EVQLVESGGGLIQPGGSLRLSCEASGFTFRNYEMNWVRQAPGKGLEWVSYISSSGNMKDYAESVKGRFTISRDNV
KNSLQLQMNSLRVEDTAVYYCARDEFPYGMDVWGQGTTVTVSS
HCDR1:
(SEQ ID NO: 213)
GFTFRNYE
HCDR2:
(SEQ ID NO: 214)
ISSSGNMK
HCDR3:
(SEQ ID NO: 215)
ARDEFPYGMDV
LCVR (V_{L}) Nucleotide Sequence
(SEQ ID NO: 216)
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAA
LCVR (V_{L}) Amino Acid Sequence
(SEQ ID NO: 217)
DIQMTQSPSSLSASVGDRVTITCRASQSISSYLNWYQQKPGKAPKLLIYAASSLQSGVPSRFSGSGSGTDETLTI
SSLQPEDFATYYCQQSYSTPPITFGQGTRLEIK
LCDR1:
(SEQ ID NO: 218)
QSISSY
LCDR2:
(SEQ ID NO: 219)
AAS
LCDR3:
(SEQ ID NO: 220)
QQSYSTPPIT
12847B
HCVR (V_{H}) Nucleotide Sequence
(SEQ ID NO: 221)
GAAGTGCAGCTGGTGGAGTCTGGGGGAGGCTTGGTTCAGCCTGGCAGGTCCCTGAGACTCTCCTGTGCAGCCTCT
GGATTCACCTTTGATGATTATGCCATGAACTGGGTCCGGCAAGCTCCAGGGAAGGGCCTGGAGTGGGTCTCAGGT
ATTAGTTGGAGTAGTGGTAGCATGGACTATGCGGACTCTGTGAAGGGCCGATTCACCATCTCCAGAGACAACGCC
AAAAACTCCCTGTATCTGCAAATGAACAGTCTGAGAACTGAGGACACGGCCTTATATTACTGTGCAAAAGCTAGG
GAAGTTGGAGACTACTACGGTATGGACGTCTGGGGCCAAGGGACCACGGTCACCGTCTCCTCA
HCVR (V_{H}) Amino Acid Sequence
(SEQ ID NO: 222)
EVQLVESGGGLVQPGRSLRLSCAASGFTEDDYAMNWVRQAPGKGLEWVSGISWSSGSMDYADSVKGRFTISRDNA
KNSLYLQMNSLRTEDTALYYCAKAREVGDYYGMDVWGQGTTVTVSS
HCDR1:
(SEQ ID NO: 223)
GFTFDDYA
HCDR2:
(SEQ ID NO: 224)
ISWSSGSM
HCDR3:
(SEQ ID NO: 225)
AKAREVGDYYGMDV
LCVR (V_{L}) Nucleotide Sequence
(SEQ ID NO: 226)
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAA
LCVR (V_{L}) Amino Acid Sequence
(SEQ ID NO: 227)
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIK
LCDR1:
(SEQ ID NO: 228)
QSISSY
LCDR2:
(SEQ ID NO: 229)
AAS
LCDR3:
(SEQ ID NO: 230)
QQSYSTPPIT
12848B
HCVR (V_{H}) Nucleotide Sequence
(SEQ ID NO: 231)
GAAGTGCAGCTGGTGGAGTCTGGGGGAGGCTTGGTACAGCCTGGCAGGTCCCTGACACTCTCCTGTGCAGCCTCT
GGATTCACCTTTGATAATTTTGGCATGCACTGGGTCCGGCAAGGTCCAGGGAAGGGCCTGGAATGGGTCTCAGGT
CTTACTTGGAATAGTGGTGTCATAGGCTATGCGGACTCTGTGAAGGGCCGATTCACCATCTCCAGAGACAACGCC
AAGAACTCCCTGTATCTGCAAATGAACAGTCTGAGACCTGAGGACACGGCCTTATATTACTGTGCAAAAGATATA
CGGAATTACGGCCCCTTTGACTACTGGGGCCAGGGAACCCTGGTCACCGTCTCCTCA
HCVR (V_{H}) Amino Acid Sequence
(SEQ ID NO: 232)
EVQLVESGGGLVQPGRSLTLSCAASGFTFDNFGMHWVRQGPGKGLEWVSGLTWNSGVIGYADSVKGRFTISRDNA
KNSLYLQMNSLRPEDTALYYCAKDIRNYGPFDYWGQGTLVTVSS
HCDR1:
(SEQ ID NO: 233)
GFTFDNFG
HCDR2:
(SEQ ID NO: 234)
LTWNSGVI
HCDR3:
(SEQ ID NO: 235)
AKDIRNYGPFDY
LCVR (V_{L}) Nucleotide Sequence
(SEQ ID NO: 236)
GAAATTGTGTTGACGCAGTCTCCAGGCACCCTGTCTTTGTCTCCAGGGGAAAGAGCCACCCTCTCCTGCAGGGCC
AGTCAGAGTGTTAGCAGCAGCTACTTAGCCTGGTACCAGCAGAAACCTGGCCAGGCTCCCAGGCTCCTCATCTAT
GGTGCATCCAGCAGGGCCACTGGCATCCCAGACAGGTTCAGTGGCAGTGGGTCTGGGACAGACTTCACTCTCACC
ATCAGCAGACTGGAGCCTGAAGATTTTGCAGTGTATTACTGTCAGCAGTATGGTAGCTCACCTTGGACGTTCGGC
CAAGGGACCAAGGTGGAAATCAAA
LCVR (V_{L}) Amino Acid Sequence
(SEQ ID NO: 237)
EIVLTQSPGTLSLSPGERATLSCRASQSVSSSYLAWYQQKPGQAPRLLIYGASSRATGIPDRFSGSGSGTDFTLT
ISRLEPEDFAVYYCQQYGSSPWTFGQGTKVEIK
LCDR1:
(SEQ ID NO: 238)
QSVSSSY
LCDR2:
(SEQ ID NO: 239)
GAS
LCDR3:
(SEQ ID NO: 240)
QQYGSSPWT
12843B
HCVR (V_{H}) Nucleotide Sequence
(SEQ ID NO: 241)
GAGGTGCAGCTGGTGGAGTCTGGGGGAGGCTTAGTACAGCCTGGAGGGTCCCTAAGACTCTCCTGTGCAGCCTCT
GGATTCACCTTCAATATTTTTGAAATGAACTGGGTCCGCCAGGCTCCAGGGAAGGGGCTGGAGTGGATTTCCTAC
ATTAGTAGTCGTGGAACTACCACATACTACGCAGACTCTGTGAGGGGCCGATTCACCATCTCCAGAGACAACGCC
AAGAACTCACTGTATCTGCAAATGAACAGCCTGAGAGCCGAGGACACGGCTGTTTATTACTGTGCGAGAGATTAT
GAAGCAACAATCCCTTTTGACTTCTGGGGCCAGGGAACCCTGGTCACCGTCTCCTCA
HCVR (V_{H}) Amino Acid Sequence
(SEQ ID NO: 242)
EVQLVESGGGLVQPGGSLRLSCAASGFTENIFEMNWVRQAPGKGLEWISYISSRGTTTYYADSVRGRFTISRDNA
KNSLYLQMNSLRAEDTAVYYCARDYEATIPFDFWGQGTLVTVSS
HCDR1:
(SEQ ID NO: 243)
GFTFNIFE
HCDR2:
(SEQ ID NO: 244)
ISSRGTTT
HCDR3:
(SEQ ID NO: 245)
ARDYEATIPFDF
LCVR (V_{L}) Nucleotide Sequence
(SEQ ID NO: 246)
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAA
LCVR (V_{L}) Amino Acid Sequence
(SEQ ID NO: 247)
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIK
LCDR1:
(SEQ ID NO: 248)
QSISSY
LCDR2:
(SEQ ID NO: 249)
AAS
LCDR3:
(SEQ ID NO: 250)
QQSYSTPPIT
12844B
HCVR (V_{H}) Nucleotide Sequence
(SEQ ID NO: 251)
GAGGTGCAGCTGGTGGAGTCTGGGGGAAGTGTGGTACGGCCTGGGGGGTCCCTGAGACTCTCCTGTGAAGCCTCT
GGATTCACCTTTGATGATTATGGCATGAGCTGGGTCCGCCAAGATCCAGGGAAGGGGCTGGAGTGGGTCTCTGGT
ATTAATTGGAATGGTGATAGAACAAATTATGCAGACTCTGTGAAGGGCCGATTCATCATTTCCAGAGACAACGCC
AAGAACTCTGTGTATCTACAAATGAACAGTCTGAGAGCGGAGGACTCGGCCTTGTATCACTGTGCGAGAGATCAG
GGACTCGGAGTGGCAGCTACCCTTGACTACTGGGGCCAGGGAACCCTGGTCACCGTCTCCTCA
HCVR (V_{H}) Amino Acid Sequence
(SEQ ID NO: 252)
EVQLVESGGSVVRPGGSLRLSCEASGFTFDDYGMSWVRQDPGKGLEWVSGINWNGDRTNYADSVKGRFIISRDNA
KNSVYLQMNSLRAEDSALYHCARDQGLGVAATLDYWGQGTLVTVSS
or
(SEQ ID NO: 482)
EVQLVESGGSVVRPGGSLRLSCEASGFTFDDYGMSWVRQDPGKGLEWVSGINWNGDRTNYADSVKGRFIISRDNA
KNSVYLQMNSLRAEDSALYHCARDQGLGVAATLDYWGQGTMVTVSS
HCDR1:
(SEQ ID NO: 253)
GFTFDDYG
HCDR2:
(SEQ ID NO: 254)
INWNGDRT
HCDR3:
(SEQ ID NO: 255)
ARDQGLGVAATLDY
LCVR (V_{L}) Nucleotide Sequence
(SEQ ID NO: 256)
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAA
LCVR (V_{L}) Amino Acid Sequence
(SEQ ID NO: 257)
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIK
LCDR1:
(SEQ ID NO: 258)
QSISSY
LCDR2:
(SEQ ID NO: 259)
AAS
LCDR3:
(SEQ ID NO: 260)
QQSYSTPPIT
12845B
HCVR (V_{H}) Nucleotide Sequence
(SEQ ID NO: 261)
GAGGTGCAGCTGGTGGAGTCTGGGGGAGGCTTGGTACAGCCTGGAGGGTCCCTGAGACTCTCCTGTGCAGCCTCT
GGATTCACCGTCAGTAATTATGAAATGAACTGGGTCCGCCAGGCTCCAGGGAAGGGGCTGGAGTGGGTTTCATAC
ATTAGTAGTAGTACCAGTAACATATACTACGCAGACTCTGTGAAGGGCCGATTCACCATCTCCAGAGACAACGCC
GAGAACTCACTGTATCTGCAGATGAACAGCCTGAGAGTCGAGGACACGGCTGTTTATTACTGTGTGAGAGATGGG
ATTGTAGTAGTTCCAGTTGGTCGTGGATACTACTATTACGGTTTGGACGTCTGGGGCCAAGGGACCACGGTCACC
GTCTCCTCA
HCVR (V_{H}) Amino Acid Sequence
(SEQ ID NO: 262)
EVQLVESGGGLVQPGGSLRLSCAASGFTVSNYEMNWVRQAPGKGLEWVSYISSSTSNIYYADSVKGRFTISRDNA
ENSLYLQMNSLRVEDTAVYYCVRDGIVVVPVGRGYYYYGLDVWGQGTTVTVSS
HCDR1:
(SEQ ID NO: 263)
GFTVSNYE
HCDR2:
(SEQ ID NO: 264)
ISSSTSNI
HCDR3:
(SEQ ID NO: 265)
VRDGIVVVPVGRGYYYYGLDV
LCVR (V_{L}) Nucleotide Sequence
(SEQ ID NO: 266)
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAA
LCVR (V_{L}) Amino Acid Sequence
(SEQ ID NO: 267)
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIK
LCDR1:
(SEQ ID NO: 268)
QSISSY
LCDR2:
(SEQ ID NO: 269)
AAS
LCDR3:
(SEQ ID NO: 270)
QQSYSTPPIT
12839B
HCVR (V_{H}) Nucleotide Sequence
(SEQ ID NO: 271)
CAGGTGCAGCTGGTGGAGTCTGGGGGAGGCGTGGTCCAGCCTGGAAGGTCCCTGAGACTCTCCTGCGCAGCCTCT
GGATTCCCCTTTAGTAATTATGTCATGTATTGGGTCCGCCAGGCTCCAGGCAAGGGGCTGGAGTGGGTGGCTCTT
ATTTTTTTTGACGGAAAGAAAAACTATCATGCAGACTCCGTGAAGGGCCGATTCACCATAACCAGAGACAATTCC
AAAAATATGTTATATCTGCAAATGAACAGCCTGAGACCTGAGGACGCGGCTGTGTATTACTGTGCGAAAATCCAT
TGTCCTAATGGTGTATGTTACAAGGGGTATTACGGAATGGACGTCTGGGGCCAAGGGACCACGGTCACCGTCTCC
TCA
HCVR (V_{H}) Amino Acid Sequence
(SEQ ID NO: 272)
QVQLVESGGGVVQPGRSLRLSCAASGFPFSNYVMYWVRQAPGKGLEWVALIFFDGKKNYHADSVKGRFTITRDNS
KNMLYLQMNSLRPEDAAVYYCAKIHCPNGVCYKGYYGMDVWGQGTTVTVSS
HCDR1:
(SEQ ID NO: 273)
GFPFSNYV
HCDR2:
(SEQ ID NO: 274)
IFFDGKKN
HCDR3:
(SEQ ID NO: 275)
AKIHCPNGVCYKGYYGMDV
LCVR (V_{L}) Nucleotide Sequence
(SEQ ID NO: 276)
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAA
LCVR (V_{L}) Amino Acid Sequence
(SEQ ID NO: 277)
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIK
LCDR1:
(SEQ ID NO: 278)
QSISSY
LCDR2:
(SEQ ID NO: 279)
AAS
LCDR3:
(SEQ ID NO: 280)
QQSYSTPPIT
12841B
HCVR (V_{H}) Nucleotide Sequence
(SEQ ID NO: 281)
GAGGTGCAGCTGGTGGAGTCTGGGGGAGGCTTGGTCCAGCCTGGGGGGTCCCTAAGACTCTCCTGTGCAGCCTCT
GGATTCACCTTTAGTAACTATTGGATGAACTGGGTCCGCCAGGCTCCAGGGAAGGGACTGGAGTGGGTGGCCAAT
ATAAAAGAAGATGGAGGTAAGAAATTGTATGTGGACTCTGTGAAGGGCCGATTCACCATCTCCAGAGACAACGCC
AAGAACTCACTGTTTCTGCAAATGAACAGCCTGAGAGCCGAGGACACGGCTGTGTATTATTGTGCGAGAGAAGAT
ACAACTTTGGTTGTGGACTACTACTACTACGGTATGGACGTCTGGGGCCAAGGGACCACGGTCACCGTCTCCTCA
HCVR (V_{H}) Amino Acid Sequence
(SEQ ID NO: 282)
EVQLVESGGGLVQPGGSLRLSCAASGFTFSNYWMNWVRQAPGKGLEWVANIKEDGGKKLYVDSVKGRFTISRDNA
KNSLFLQMNSLRAEDTAVYYCAREDTTLVVDYYYYGMDVWGQGTTVTVSS
HCDR1:
(SEQ ID NO: 283)
GFTFSNYW
HCDR2:
(SEQ ID NO: 284)
IKEDGGKK
HCDR3:
(SEQ ID NO: 285)
AREDTTLVVDYYYYGMDV
LCVR (V_{L}) Nucleotide Sequence
(SEQ ID NO: 286)
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAA
LCVR (V_{L}) Amino Acid Sequence
(SEQ ID NO: 287)
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIK
LCDR1:
(SEQ ID NO: 288)
QSISSY
LCDR2:
(SEQ ID NO: 289)
AAS
LCDR3:
(SEQ ID NO: 290)
QQSYSTPPIT
12850B
HCVR (V_{H}) Nucleotide Sequence
(SEQ ID NO: 291)
CAGGTCCAGCTGGTGCAGTCTGGGGCTGAGGTGAAGAAGCCTGGGTCCTCGGTGAAGGTCTCCTGCAAGGCTTCT
GGAGGCACCTTCAACACCTATGCTATCACCTGGGTGCGACAGGCCCCTGGACAAGGGCTTGAATGGATGGGGGGA
ATCATCCCTATCTCTGGCATAGCAGAGTACGCACAGAAGTTCCAGGGCAGAGTCACGATCACCACGGATGACTCC
TCGACCACAGCCTACATGGAACTGAACAGTCTGAGATCTGAGGACACGGCCGTGTATTACTGTGCGAGCTGGAAC
TACGCACTCTACTACTTCTACGGTATGGACGTCTGGGGCCGAGGGACCACGGTCACCGTCTCCTCA
HCVR (V_{H}) Amino Acid Sequence
(SEQ ID NO: 292)
QVQLVQSGAEVKKPGSSVKVSCKASGGTFNTYAITWVRQAPGQGLEWMGGIIPISGIAEYAQKFQGRVTITTDDS
STTAYMELNSLRSEDTAVYYCASWNYALYYFYGMDVWGRGTTVTVSS
HCDR1:
(SEQ ID NO: 293)
GGTFNTYA
HCDR2:
(SEQ ID NO: 294)
IIPISGIA
HCDR3:
(SEQ ID NO: 295)
ASWNYALYYFYGMDV
LCVR (V_{L}) Nucleotide Sequence
(SEQ ID NO: 296)
GAAATTGTGTTGACGCAGTCTCCAGGCACCCTGTCTTTGTCTCCAGGGGAAAGAGCCACCCTCTCCTGCAGGGCC
AGTCAGAGTGTTAGCAGCAGCTACTTAGCCTGGTACCAGCAGAAACCTGGCCAGGCTCCCAGGCTCCTCATCTAT
GGTGCATCCAGCAGGGCCACTGGCATCCCAGACAGGTTCAGTGGCAGTGGGTCTGGGACAGACTTCACTCTCACC
ATCAGCAGACTGGAGCCTGAAGATTTTGCAGTGTATTACTGTCAGCAGTATGGTAGCTCACCTTGGACGTTCGGC
CAAGGGACCAAGGTGGAAATCAAA
LCVR (V_{L}) Amino Acid Sequence
(SEQ ID NO: 297)
EIVLTQSPGTLSLSPGERATLSCRASQSVSSSYLAWYQQKPGQAPRLLIYGASSRATGIPDRESGSGSGTDFTLT
ISRLEPEDFAVYYCQQYGSSPWTFGQGTKVEIK
LCDR1:
(SEQ ID NO: 298)
QSVSSSY
LCDR2:
(SEQ ID NO: 299)
GAS
LCDR3:
(SEQ ID NO: 300)
QQYGSSPWT
69261
HCVR (V_{H}) Nucleotide Sequence
(SEQ ID NO: 301)
CAGGTGCAGCTGGTGGAGTCTGGGGGAGGCTTGGTCAAGCCTGGAGGGTCCCTGAGACTCTCCTGTGCAGCCTCT
GGATTCACCTTCAGTGTCTATTACATGAACTGGATCCGCCAGGCTCCAGGGAAGGGCCTGGAGTGGGTTTCATAC
ATTAGTAGTAGTGGTAGTACCATATACTACGCAGACTCTGTGAAGGGCCGATTCACCATCTCCAGGGACAACGCC
AAGAACTCACTGTATCTCCAAATGAACAGTCTGAGAGCCGAGGACACGGCCGTATATTACTGTGGGAGAGAAGGG
TATAGTGGGACTTATTCTTATTACGGTATGGACGTCTGGGGCCAAGGGACCACGGTCACCGTCTCCTCA
HCVR (V_{H}) Amino Acid Sequence
(SEQ ID NO: 302)
QVQLVESGGGLVKPGGSLRLSCAASGFTFSVYYMNWIRQAPGKGLEWVSYISSSGSTIYYADSVKGRFTISRDNA
KNSLYLQMNSLRAEDTAVYYCGREGYSGTYSYYGMDVWGQGTTVTVSS
or
(SEQ ID NO: 483)
EVQLVESGGGLVKPGGSLRLSCAASGFTFSVYYMNWIRQAPGKGLEWVSYISSSGSTIYYADSVKGRFTISRDNA
KNSLYLQMNSLRAEDTAVYYCGREGYSGTYSYYGMDVWGQGTTVTVSS
HCDR1:
(SEQ ID NO: 303)
GFTFSVYY
HCDR2:
(SEQ ID NO: 304)
ISSSGSTI
HCDR3:
(SEQ ID NO: 305)
GREGYSGTYSYYGMDV
LCVR (V_{L}) Nucleotide Sequence
(SEQ ID NO: 306)
GATATTGTGATGACTCAGTCTCCACTCTCCCTGCCCGTCACCCCTGGAGAGCCGGCCTCCATCTCCTGCAGGTCT
AGTCAGAGCCTCCTGCATAGTAATGGATACAACTATTTGGATTGGTACCTGCAGAAGCCAGGGCAGTCTCCACAG
TTCCTGATCTATTTGGGTTCTAATCGGGCCTCCGGGGTCCCTGACAGGTTCAGTGGCAGTGGATCAGGCACAGAT
TTTACACTGAAAATCAACAGAGTGGAGGCTGAGGATGTTGGGGTTTATTACTGCATGCAAGCTCTACAAACTCCG
TACACTTTTGGCCAGGGGACCAAGCTGGAGATCAAA
LCVR (V_{L}) Amino Acid Sequence
(SEQ ID NO: 307)
DIVMTQSPLSLPVTPGEPASISCRSSQSLLHSNGYNYLDWYLQKPGQSPQFLIYLGSNRASGVPDRFSGSGSGTD
FTLKINRVEAEDVGVYYCMQALQTPYTFGQGTKLEIK
or
(SEQ ID NO: 527)
DIQLTQSPLSLPVTPGEPASISCRSSQSLLHSNGYNYLDWYLQKPGQSPQFLIYLGSNRASGVPDRFSGSGSGTD
FTLKINRVEAEDVGVYYCMQALQTPYTFGQGTKVEIK
LCDR1:
(SEQ ID NO: 308)
QSLLHSNGYNY
LCDR2:
(SEQ ID NO: 309)
LGS
LCDR3:
(SEQ ID NO: 310)
MQALQTPYT
69263
HCVR (V_{H}) Nucleotide Sequence
(SEQ ID NO: 311)
GAAGTGCAGCTGGTGGAGTCTGGGGGAGGGTTGGTACAGCCTGGCAGGTCCCTGAGACTCTCCTGTGCAGTCTCT
GGATTCACCTTTGATGATTATGCCATGCACTGGGTCCGGCAAGCTCCAGGGAAGGGCCTGGAGTGGGTCTCAGGT
ATTAGTTGGAATAGTGGTACCAGAGGATATGCGGACTCTGTGAAGGGCCGATTCACCATCTCCAGAGACAACGCC
AAGAACTCCCTGTATCTGCAAATGAACAGTCTGAGAGGTGAGGACACGGCCTTGTATTACTGTGTAAAAGATATT
ACGATATCCCCCAACTACTACGGTATGGACGTCTGGGGCCAAGGGACCACGGTCACCGTCTCCTCA
HCVR (V_{H}) Amino Acid Sequence
(SEQ ID NO: 312)
EVQLVESGGGLVQPGRSLRLSCAVSGFTFDDYAMHWVRQAPGKGLEWVSGISWNSGTRGYADSVKGRFTISRDNA
KNSLYLQMNSLRGEDTALYYCVKDITISPNYYGMDVWGQGTTVTVSS
HCDR1:
(SEQ ID NO: 313)
GFTFDDYA
HCDR2:
(SEQ ID NO: 314)
ISWNSGTR
HCDR3:
(SEQ ID NO: 315)
VKDITISPNYYGMDV
LCVR (V_{L}) Nucleotide Sequence
(SEQ ID NO: 316)
GACATCCAGATGACCCAGTCTCCATCCTCCCTGTCTGCATCTGTAGGAGACAGAGTCACCATCACTTGCCGGGCG
AGTCAGGACATTAGCCATTATTCAGCCTGGTATCAGCAGAAACCAGGGAAACTTCCTAACCTCCTGATCTATGCT
GCATCCACTTTGCAATCAGGGGTCCCATCTCGGTTCAGTGGCAGTGGATCTGGGACAGATTTCTCTCTCACCACC
AGCAGCCTGCAGCCTGAAGATGTTGCAACTTATTACTGTCAAAAGTATAACAGTGTCCCTCTCACTTTCGGCGGA
GGGACCAAGGTGGAGATCAAA
LCVR (V_{L}) Amino Acid Sequence
(SEQ ID NO: 317)
DIQMTQSPSSLSASVGDRVTITCRASQDISHYSAWYQQKPGKLPNLLIYAASTLQSGVPSRFSGSGSGTDESLTT
SSLQPEDVATYYCQKYNSVPLTFGGGTKVEIK
or
(SEQ ID NO: 484)
DIQLTQSPSSLSASVGDRVTITCRASQDISHYSAWYQQKPGKLPNLLIYAASTLQSGVPSRFSGSGSGTDESLTT
SSLQPEDVATYYCQKYNSVPLTFGGRTKVEIK
LCDR1:
(SEQ ID NO: 318)
QDISHY
LCDR2:
(SEQ ID NO: 319)
AAS
LCDR3:
(SEQ ID NO: 320)
QKYNSVPLT

As discussed, an anti-hTfR scFv protein-drug conjugate (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or69263) comprises an optional signal peptide (e.g., mROR signal sequence), connected to an scFv (e.g., including a V_{L} and a V_{H} optionally connected by a linker), connected to an option linker, connected to a molecular cargo wherein, for example:
(I)
the optional signal peptide is, for example, the signal peptide from Mus musculus Ror1 (e.g., consisting of the amino acids MHRPRRRGTRPPPLALLAALLLAARGADA (SEQ ID NO: 528));
(II)
the scFv comprises (i) a heavy chain variable region that comprises the HCDR1, HCDR2 and HCDR3 of a HCVR comprising the amino acid sequence set forth in SEQ ID NO: 2; 462; 12; 463; 22; 464; 32; 42; 52; 467; 62; 492; 72; 470; 82; 92; 472; 102; 112; 473; 122; 132; 142; 475; 152; 162; 477; 172; 182; 478; 192; 480; 202; 481; 212; 222; 232; 242; 252; 482; 262; 272; 282; 292; 302; 483 or 312;
and/or
(ii) a light chain variable region that comprises the LCDR1, LCDR2 and LCDR3 of a LCVR comprising the amino acid sequence set forth in SEQ ID NO: 7; 17; 27; 37; 465; 47; 466; 57; 468; 67; 469; 77; 471; 87; 97; 107; 117; 474; 127; 137; 147; 476; 157; 167; 177; 187; 479; 197; 207; 217; 227; 237; 247; 257; 267; 277; 287; 297; 307; 527; 317 or 484;
or the scFv comprises:
(1) a HCVR comprising the HCDR1, HCDR2 and HCDR3 of a HCVR that comprises the amino acid sequence set forth in SEQ ID NO: 2 or 462 (or a variant thereof); and a LCVR comprising the LCDR1, LCDR2 and LCDR3 of a LCVR that comprises the amino acid sequence set forth in SEQ ID NO: 7 (or a variant thereof);
(2) a HCVR comprising the HCDR1, HCDR2 and HCDR3 of a HCVR that comprises the amino acid sequence set forth in SEQ ID NO: 12 or 463 (or a variant thereof); and a LCVR comprising the LCDR1, LCDR2 and LCDR3 of a LCVR that comprises the amino acid sequence set forth in SEQ ID NO: 17 (or a variant thereof);
(3) a HCVR comprising the HCDR1, HCDR2 and HCDR3 of a HCVR that comprises the amino acid sequence set forth in SEQ ID NO: 22 or 464 (or a variant thereof); and a LCVR comprising the LCDR1, LCDR2 and LCDR3 of a LCVR that comprises the amino acid sequence set forth in SEQ ID NO: 27 (or a variant thereof);
(4) a HCVR comprising the HCDR1, HCDR2 and HCDR3 of a HCVR that comprises the amino acid sequence set forth in SEQ ID NO: 32 (or a variant thereof); and a LCVR comprising the LCDR1, LCDR2 and LCDR3 of a LCVR that comprises the amino acid sequence set forth in SEQ ID NO: 37 or 465 (or a variant thereof);
(5) a HCVR comprising the HCDR1, HCDR2 and HCDR3 of a HCVR that comprises the amino acid sequence set forth in SEQ ID NO: 42 (or a variant thereof); and a LCVR comprising the LCDR1, LCDR2 and LCDR3 of a LCVR that comprises the amino acid sequence set forth in SEQ ID NO: 47 or 466 (or a variant thereof);
(6) a HCVR comprising the HCDR1, HCDR2 and HCDR3 of a HCVR that comprises the amino acid sequence set forth in SEQ ID NO: 52 or 467 (or a variant thereof); and a LCVR comprising the LCDR1, LCDR2 and LCDR3 of a LCVR that comprises the amino acid sequence set forth in SEQ ID NO: 57 or 468 (or a variant thereof);
(7) a HCVR comprising the HCDR1, HCDR2 and HCDR3 of a HCVR that comprises the amino acid sequence set forth in SEQ ID NO: 62 or 492 (or a variant thereof); and a LCVR comprising the LCDR1, LCDR2 and LCDR3 of a LCVR that comprises the amino acid sequence set forth in SEQ ID NO: 67 or 469 (or a variant thereof);
(8) a HCVR comprising the HCDR1, HCDR2 and HCDR3 of a HCVR that comprises the amino acid sequence set forth in SEQ ID NO: 72 or 470 (or a variant thereof); and a LCVR comprising the LCDR1, LCDR2 and LCDR3 of a LCVR that comprises the amino acid sequence set forth in SEQ ID NO: 77 or 471 (or a variant thereof);
(9) a HCVR comprising the HCDR1, HCDR2 and HCDR3 of a HCVR that comprises the amino acid sequence set forth in SEQ ID NO: 82 (or a variant thereof); and a LCVR comprising the LCDR1, LCDR2 and LCDR3 of a LCVR that comprises the amino acid sequence set forth in SEQ ID NO: 87 (or a variant thereof);
(10) a HCVR comprising the HCDR1, HCDR2 and HCDR3 of a HCVR that comprises the amino acid sequence set forth in SEQ ID NO: 92 or 472 (or a variant thereof); and a LCVR comprising the LCDR1, LCDR2 and LCDR3 of a LCVR that comprises the amino acid sequence set forth in SEQ ID NO: 97 (or a variant thereof);
(11) a HCVR comprising the HCDR1, HCDR2 and HCDR3 of a HCVR that comprises the amino acid sequence set forth in SEQ ID NO: 102 (or a variant thereof); and a LCVR comprising the LCDR1, LCDR2 and LCDR3 of a LCVR that comprises the amino acid sequence set forth in SEQ ID NO: 107 (or a variant thereof);
(12) a HCVR comprising the HCDR1, HCDR2 and HCDR3 of a HCVR that comprises the amino acid sequence set forth in SEQ ID NO: 112 or 473 (or a variant thereof); and a LCVR comprising the LCDR1, LCDR2 and LCDR3 of a LCVR that comprises the amino acid sequence set forth in SEQ ID NO: 117 or 474 (or a variant thereof);
(13) a HCVR comprising the HCDR1, HCDR2 and HCDR3 of a HCVR that comprises the amino acid sequence set forth in SEQ ID NO: 122 (or a variant thereof); and a LCVR comprising the LCDR1, LCDR2 and LCDR3 of a LCVR that comprises the amino acid sequence set forth in SEQ ID NO: 127 (or a variant thereof);
(14) a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof);
(15) a HCVR comprising the HCDR1, HCDR2 and HCDR3 of a HCVR that comprises the amino acid sequence set forth in SEQ ID NO: 142 or 475 (or a variant thereof); and a LCVR comprising the LCDR1, LCDR2 and LCDR3 of a LCVR that comprises the amino acid sequence set forth in SEQ ID NO: 147 or 476 (or a variant thereof);
(16) a HCVR comprising the HCDR1, HCDR2 and HCDR3 of a HCVR that comprises the amino acid sequence set forth in SEQ ID NO: 152 (or a variant thereof); and a LCVR comprising the LCDR1, LCDR2 and LCDR3 of a LCVR that comprises the amino acid sequence set forth in SEQ ID NO: 157 (or a variant thereof);
(17) a HCVR comprising the HCDR1, HCDR2 and HCDR3 of a HCVR that comprises the amino acid sequence set forth in SEQ ID NO: 162 or 477 (or a variant thereof); and a LCVR comprising the LCDR1, LCDR2 and LCDR3 of a LCVR that comprises the amino acid sequence set forth in SEQ ID NO: 167 (or a variant thereof);
(18) a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof);
(19) a HCVR comprising the HCDR1, HCDR2 and HCDR3 of a HCVR that comprises the amino acid sequence set forth in SEQ ID NO: 182 or 478 (or a variant thereof); and a LCVR comprising the LCDR1, LCDR2 and LCDR3 of a LCVR that comprises the amino acid sequence set forth in SEQ ID NO: 187 or 479 (or a variant thereof);
(20) a HCVR comprising the HCDR1, HCDR2 and HCDR3 of a HCVR that comprises the amino acid sequence set forth in SEQ ID NO: 192 or 480 (or a variant thereof); and a LCVR comprising the LCDR1, LCDR2 and LCDR3 of a LCVR that comprises the amino acid sequence set forth in SEQ ID NO: 197 (or a variant thereof);
(21) a HCVR comprising the HCDR1, HCDR2 and HCDR3 of a HCVR that comprises the amino acid sequence set forth in SEQ ID NO: 202 or 481 (or a variant thereof); and a LCVR comprising the LCDR1, LCDR2 and LCDR3 of a LCVR that comprises the amino acid sequence set forth in SEQ ID NO: 207 (or a variant thereof);
(22) a HCVR comprising the HCDR1, HCDR2 and HCDR3 of a HCVR that comprises the amino acid sequence set forth in SEQ ID NO: 212 (or a variant thereof); and a LCVR comprising the LCDR1, LCDR2 and LCDR3 of a LCVR that comprises the amino acid sequence set forth in SEQ ID NO: 217 (or a variant thereof);
(23) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof);
(24) a HCVR comprising the HCDR1, HCDR2 and HCDR3 of a HCVR that comprises the amino acid sequence set forth in SEQ ID NO: 232 (or a variant thereof); and a LCVR comprising the LCDR1, LCDR2 and LCDR3 of a LCVR that comprises the amino acid sequence set forth in SEQ ID NO: 237 (or a variant thereof);
(25) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof);
(26) a HCVR comprising the HCDR1, HCDR2 and HCDR3 of a HCVR that comprises the amino acid sequence set forth in SEQ ID NO: 252 or 482 (or a variant thereof); and a LCVR comprising the LCDR1, LCDR2 and LCDR3 of a LCVR that comprises the amino acid sequence set forth in SEQ ID NO: 257 (or a variant thereof);
(27) a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof);
(28) a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof);
(29) a HCVR comprising the HCDR1, HCDR2 and HCDR3 of a HCVR that comprises the amino acid sequence set forth in SEQ ID NO: 282 (or a variant thereof); and a LCVR comprising the LCDR1, LCDR2 and LCDR3 of a LCVR that comprises the amino acid sequence set forth in SEQ ID NO: 287 (or a variant thereof);
(30) a HCVR comprising the HCDR1, HCDR2 and HCDR3 of a HCVR that comprises the amino acid sequence set forth in SEQ ID NO: 292 (or a variant thereof); and a LCVR comprising the LCDR1, LCDR2 and LCDR3 of a LCVR that comprises the amino acid sequence set forth in SEQ ID NO: 297 (or a variant thereof);
(31) a HCVR comprising the HCDR1, HCDR2 and HCDR3 of a HCVR that comprises the amino acid sequence set forth in SEQ ID NO: 302 or 483 (or a variant thereof); and a LCVR comprising the LCDR1, LCDR2 and LCDR3 of a LCVR that comprises the amino acid sequence set forth in SEQ ID NO: 307 or 527 (or a variant thereof);
(32) a HCVR comprising the HCDR1, HCDR2 and HCDR3 of a HCVR that comprises the amino acid sequence set forth in SEQ ID NO: 312 (or a variant thereof); and a LCVR comprising the LCDR1, LCDR2 and LCDR3 of a LCVR that comprises the amino acid sequence set forth in SEQ ID NO: 317 or 484 (or a variant thereof);
or the scFv comprises:
(a) a HCVR that comprises:
an HCDR1 comprising the amino acid sequence set forth in SEQ ID NO: 3 (or a variant thereof),
an HCDR2 comprising the amino acid sequence set forth in SEQ ID NO: 4 (or a variant thereof), and
an HCDR3 comprising the amino acid sequence set forth in SEQ ID NO: 5 (or a variant thereof); and
a LCVR that comprises:
an LCDR1 comprising the amino acid sequence set forth in SEQ ID NO: 8 (or a variant thereof),
an LCDR2 comprising the amino acid sequence set forth in SEQ ID NO: 9 (or a variant thereof), and
an LCDR3 comprising the amino acid sequence set forth in SEQ ID NO: 10 (or a variant thereof);
(b) a HCVR that comprises:
an HCDR1 comprising the amino acid sequence set forth in SEQ ID NO: 13 (or a variant thereof),
an HCDR2 comprising the amino acid sequence set forth in SEQ ID NO: 14 (or a variant thereof), and
an HCDR3 comprising the amino acid sequence set forth in SEQ ID NO: 15 (or a variant thereof); and
a LCVR that comprises:
an LCDR1 comprising the amino acid sequence set forth in SEQ ID NO: 18 (or a variant thereof),
an LCDR2 comprising the amino acid sequence set forth in SEQ ID NO: 19 (or a variant thereof), and
an LCDR3 comprising the amino acid sequence set forth in SEQ ID NO: 20 (or a variant thereof);
(c) a HCVR that comprises:
an HCDR1 comprising the amino acid sequence set forth in SEQ ID NO: 23 (or a variant thereof),
an HCDR2 comprising the amino acid sequence set forth in SEQ ID NO: 24 (or a variant thereof), and
an HCDR3 comprising the amino acid sequence set forth in SEQ ID NO: 25 (or a variant thereof); and
a LCVR that comprises:
an LCDR1 comprising the amino acid sequence set forth in SEQ ID NO: 28 (or a variant thereof),
an LCDR2 comprising the amino acid sequence set forth in SEQ ID NO: 29 (or a variant thereof), and
an LCDR3 comprising the amino acid sequence set forth in SEQ ID NO: 30 (or a variant thereof);
(d) a HCVR that comprises:
an HCDR1 comprising the amino acid sequence set forth in SEQ ID NO: 33 (or a variant thereof),
an HCDR2 comprising the amino acid sequence set forth in SEQ ID NO: 34 (or a variant thereof), and
an HCDR3 comprising the amino acid sequence set forth in SEQ ID NO: 35 (or a variant thereof); and
a LCVR that comprises:
an LCDR1 comprising the amino acid sequence set forth in SEQ ID NO: 38 (or a variant thereof),
an LCDR2 comprising the amino acid sequence set forth in SEQ ID NO: 39 (or a variant thereof), and
an LCDR3 comprising the amino acid sequence set forth in SEQ ID NO: 40 (or a variant thereof);
(e) a HCVR that comprises:
an HCDR1 comprising the amino acid sequence set forth in SEQ ID NO: 43 (or a variant thereof),
an HCDR2 comprising the amino acid sequence set forth in SEQ ID NO: 44 (or a variant thereof), and
an HCDR3 comprising the amino acid sequence set forth in SEQ ID NO: 45 (or a variant thereof); and
a LCVR that comprises:
an LCDR1 comprising the amino acid sequence set forth in SEQ ID NO: 48 (or a variant thereof),
an LCDR2 comprising the amino acid sequence set forth in SEQ ID NO: 49 (or a variant thereof), and
an LCDR3 comprising the amino acid sequence set forth in SEQ ID NO: 50 (or a variant thereof);
(f) a HCVR that comprises:
an HCDR1 comprising the amino acid sequence set forth in SEQ ID NO: 53 (or a variant thereof),
an HCDR2 comprising the amino acid sequence set forth in SEQ ID NO: 54 (or a variant thereof), and
an HCDR3 comprising the amino acid sequence set forth in SEQ ID NO: 55 (or a variant thereof); and
a LCVR that comprises:
an LCDR1 comprising the amino acid sequence set forth in SEQ ID NO: 58 (or a variant thereof),
an LCDR2 comprising the amino acid sequence set forth in SEQ ID NO: 59 (or a variant thereof), and
an LCDR3 comprising the amino acid sequence set forth in SEQ ID NO: 60 (or a variant thereof);
(g) a HCVR that comprises:
an HCDR1 comprising the amino acid sequence set forth in SEQ ID NO: 63 (or a variant thereof),
an HCDR2 comprising the amino acid sequence set forth in SEQ ID NO: 64 (or a variant thereof), and
an HCDR3 comprising the amino acid sequence set forth in SEQ ID NO: 65 (or a variant thereof); and
a LCVR that comprises:
an LCDR1 comprising the amino acid sequence set forth in SEQ ID NO: 68 (or a variant thereof),
an LCDR2 comprising the amino acid sequence set forth in SEQ ID NO: 69 (or a variant thereof), and
an LCDR3 comprising the amino acid sequence set forth in SEQ ID NO: 70 (or a variant thereof);
(h) a HCVR that comprises:
an HCDR1 comprising the amino acid sequence set forth in SEQ ID NO: 73 (or a variant thereof),
an HCDR2 comprising the amino acid sequence set forth in SEQ ID NO: 74 (or a variant thereof), and
an HCDR3 comprising the amino acid sequence set forth in SEQ ID NO: 75 (or a variant thereof); and
a LCVR that comprises:
an LCDR1 comprising the amino acid sequence set forth in SEQ ID NO: 78 (or a variant thereof),
an LCDR2 comprising the amino acid sequence set forth in SEQ ID NO: 79 (or a variant thereof), and
an LCDR3 comprising the amino acid sequence set forth in SEQ ID NO: 80 (or a variant thereof);
(i) a HCVR that comprises:
an HCDR1 comprising the amino acid sequence set forth in SEQ ID NO: 83 (or a variant thereof),
an HCDR2 comprising the amino acid sequence set forth in SEQ ID NO: 84 (or a variant thereof), and
an HCDR3 comprising the amino acid sequence set forth in SEQ ID NO: 85 (or a variant thereof); and
a LCVR that comprises:
an LCDR1 comprising the amino acid sequence set forth in SEQ ID NO: 88 (or a variant thereof),
an LCDR2 comprising the amino acid sequence set forth in SEQ ID NO: 89 (or a variant thereof), and
an LCDR3 comprising the amino acid sequence set forth in SEQ ID NO: 90 (or a variant thereof);
(j) a HCVR that comprises:
an HCDR1 comprising the amino acid sequence set forth in SEQ ID NO: 93 (or a variant thereof),
an HCDR2 comprising the amino acid sequence set forth in SEQ ID NO: 94 (or a variant thereof), and
an HCDR3 comprising the amino acid sequence set forth in SEQ ID NO: 95 (or a variant thereof); and
a LCVR that comprises:
an LCDR1 comprising the amino acid sequence set forth in SEQ ID NO: 98 (or a variant thereof),
an LCDR2 comprising the amino acid sequence set forth in SEQ ID NO: 99 (or a variant thereof), and
an LCDR3 comprising the amino acid sequence set forth in SEQ ID NO: 100 (or a variant thereof);
(k) a HCVR that comprises:
an HCDR1 comprising the amino acid sequence set forth in SEQ ID NO: 103 (or a variant thereof),
an HCDR2 comprising the amino acid sequence set forth in SEQ ID NO: 104 (or a variant thereof), and
an HCDR3 comprising the amino acid sequence set forth in SEQ ID NO: 105 (or a variant thereof); and
a LCVR that comprises:
an LCDR1 comprising the amino acid sequence set forth in SEQ ID NO: 108 (or a variant thereof),
an LCDR2 comprising the amino acid sequence set forth in SEQ ID NO: 109 (or a variant thereof), and
an LCDR3 comprising the amino acid sequence set forth in SEQ ID NO: 110 (or a variant thereof);
(l) a HCVR that comprises:
an HCDR1 comprising the amino acid sequence set forth in SEQ ID NO: 113 (or a variant thereof),
an HCDR2 comprising the amino acid sequence set forth in SEQ ID NO: 114 (or a variant thereof), and
an HCDR3 comprising the amino acid sequence set forth in SEQ ID NO: 115 (or a variant thereof); and
a LCVR that comprises:
an LCDR1 comprising the amino acid sequence set forth in SEQ ID NO: 118 (or a variant thereof),
an LCDR2 comprising the amino acid sequence set forth in SEQ ID NO: 119 (or a variant thereof), and
an LCDR3 comprising the amino acid sequence set forth in SEQ ID NO: 120 (or a variant thereof);
(m) a HCVR that comprises:
an HCDR1 comprising the amino acid sequence set forth in SEQ ID NO: 123 (or a variant thereof),
an HCDR2 comprising the amino acid sequence set forth in SEQ ID NO: 124 (or a variant thereof), and
an HCDR3 comprising the amino acid sequence set forth in SEQ ID NO: 125 (or a variant thereof); and
a LCVR that comprises:
an LCDR1 comprising the amino acid sequence set forth in SEQ ID NO: 128 (or a variant thereof),
an LCDR2 comprising the amino acid sequence set forth in SEQ ID NO: 129 (or a variant thereof), and
an LCDR3 comprising the amino acid sequence set forth in SEQ ID NO: 130 (or a variant thereof);
(n) a HCVR that comprises:
an HCDR1 comprising the amino acid sequence set forth in SEQ ID NO: 133 (or a variant thereof),
an HCDR2 comprising the amino acid sequence set forth in SEQ ID NO: 134 (or a variant thereof), and
an HCDR3 comprising the amino acid sequence set forth in SEQ ID NO: 135 (or a variant thereof); and
a LCVR that comprises:
an LCDR1 comprising the amino acid sequence set forth in SEQ ID NO: 138 (or a variant thereof),
an LCDR2 comprising the amino acid sequence set forth in SEQ ID NO: 139 (or a variant thereof), and
an LCDR3 comprising the amino acid sequence set forth in SEQ ID NO: 140 (or a variant thereof);
(o) a HCVR that comprises:
an HCDR1 comprising the amino acid sequence set forth in SEQ ID NO: 143 (or a variant thereof),
an HCDR2 comprising the amino acid sequence set forth in SEQ ID NO: 144 (or a variant thereof), and
an HCDR3 comprising the amino acid sequence set forth in SEQ ID NO: 145 (or a variant thereof); and
a LCVR that comprises:
an LCDR1 comprising the amino acid sequence set forth in SEQ ID NO: 148 (or a variant thereof),
an LCDR2 comprising the amino acid sequence set forth in SEQ ID NO: 149 (or a variant thereof), and
an LCDR3 comprising the amino acid sequence set forth in SEQ ID NO: 150 (or a variant thereof);
(p) a HCVR that comprises:
an HCDR1 comprising the amino acid sequence set forth in SEQ ID NO: 153 (or a variant thereof),
an HCDR2 comprising the amino acid sequence set forth in SEQ ID NO: 154 (or a variant thereof), and
an HCDR3 comprising the amino acid sequence set forth in SEQ ID NO: 155 (or a variant thereof); and
a LCVR that comprises:
an LCDR1 comprising the amino acid sequence set forth in SEQ ID NO: 158 (or a variant thereof),
an LCDR2 comprising the amino acid sequence set forth in SEQ ID NO: 159 (or a variant thereof), and
an LCDR3 comprising the amino acid sequence set forth in SEQ ID NO: 160 (or a variant thereof);
(q) a HCVR that comprises:
an HCDR1 comprising the amino acid sequence set forth in SEQ ID NO: 163 (or a variant thereof),
an HCDR2 comprising the amino acid sequence set forth in SEQ ID NO: 164 (or a variant thereof), and
an HCDR3 comprising the amino acid sequence set forth in SEQ ID NO: 165 (or a variant thereof); and
a LCVR that comprises:
an LCDR1 comprising the amino acid sequence set forth in SEQ ID NO: 168 (or a variant thereof),
an LCDR2 comprising the amino acid sequence set forth in SEQ ID NO: 169 (or a variant thereof), and
an LCDR3 comprising the amino acid sequence set forth in SEQ ID NO: 170 (or a variant thereof);
(r) a HCVR that comprises:
an HCDR1 comprising the amino acid sequence set forth in SEQ ID NO: 173 (or a variant thereof),
an HCDR2 comprising the amino acid sequence set forth in SEQ ID NO: 174 (or a variant thereof), and
an HCDR3 comprising the amino acid sequence set forth in SEQ ID NO: 175 (or a variant thereof); and
a LCVR that comprises:
an LCDR1 comprising the amino acid sequence set forth in SEQ ID NO: 178 (or a variant thereof),
an LCDR2 comprising the amino acid sequence set forth in SEQ ID NO: 179 (or a variant thereof), and
an LCDR3 comprising the amino acid sequence set forth in SEQ ID NO: 180 (or a variant thereof);
(s) a HCVR that comprises:
an HCDR1 comprising the amino acid sequence set forth in SEQ ID NO: 183 (or a variant thereof),
an HCDR2 comprising the amino acid sequence set forth in SEQ ID NO: 184 (or a variant thereof), and
an HCDR3 comprising the amino acid sequence set forth in SEQ ID NO: 185 (or a variant thereof); and
a LCVR that comprises:
an LCDR1 comprising the amino acid sequence set forth in SEQ ID NO: 188 (or a variant thereof),
an LCDR2 comprising the amino acid sequence set forth in SEQ ID NO: 189 (or a variant thereof), and
an LCDR3 comprising the amino acid sequence set forth in SEQ ID NO: 190 (or a variant thereof);
(t) a HCVR that comprises:
an HCDR1 comprising the amino acid sequence set forth in SEQ ID NO: 193 (or a variant thereof),
an HCDR2 comprising the amino acid sequence set forth in SEQ ID NO: 194 (or a variant thereof), and
an HCDR3 comprising the amino acid sequence set forth in SEQ ID NO: 195 (or a variant thereof); and
a LCVR that comprises:
an LCDR1 comprising the amino acid sequence set forth in SEQ ID NO: 198 (or a variant thereof),
an LCDR2 comprising the amino acid sequence set forth in SEQ ID NO: 199 (or a variant thereof), and
an LCDR3 comprising the amino acid sequence set forth in SEQ ID NO: 200 (or a variant thereof);
(u) a HCVR that comprises:
an HCDR1 comprising the amino acid sequence set forth in SEQ ID NO: 203 (or a variant thereof),
an HCDR2 comprising the amino acid sequence set forth in SEQ ID NO: 204 (or a variant thereof), and
an HCDR3 comprising the amino acid sequence set forth in SEQ ID NO: 205 (or a variant thereof); and
a LCVR that comprises:
an LCDR1 comprising the amino acid sequence set forth in SEQ ID NO: 208 (or a variant thereof),
an LCDR2 comprising the amino acid sequence set forth in SEQ ID NO: 209 (or a variant thereof), and
an LCDR3 comprising the amino acid sequence set forth in SEQ ID NO: 210 (or a variant thereof);
(v) a HCVR that comprises:
an HCDR1 comprising the amino acid sequence set forth in SEQ ID NO: 213 (or a variant thereof),
an HCDR2 comprising the amino acid sequence set forth in SEQ ID NO: 214 (or a variant thereof), and
an HCDR3 comprising the amino acid sequence set forth in SEQ ID NO: 215 (or a variant thereof); and
a LCVR that comprises:
an LCDR1 comprising the amino acid sequence set forth in SEQ ID NO: 218 (or a variant thereof),
an LCDR2 comprising the amino acid sequence set forth in SEQ ID NO: 219 (or a variant thereof), and
an LCDR3 comprising the amino acid sequence set forth in SEQ ID NO: 220 (or a variant thereof);
(w) a HCVR that comprises:
an HCDR1 comprising the amino acid sequence set forth in SEQ ID NO: 223 (or a variant thereof),
an HCDR2 comprising the amino acid sequence set forth in SEQ ID NO: 224 (or a variant thereof), and
an HCDR3 comprising the amino acid sequence set forth in SEQ ID NO: 225 (or a variant thereof); and
a LCVR that comprises:
an LCDR1 comprising the amino acid sequence set forth in SEQ ID NO: 228 (or a variant thereof),
an LCDR2 comprising the amino acid sequence set forth in SEQ ID NO: 229 (or a variant thereof), and
an LCDR3 comprising the amino acid sequence set forth in SEQ ID NO: 230 (or a variant thereof);
(x) a HCVR that comprises:
an HCDR1 comprising the amino acid sequence set forth in SEQ ID NO: 233 (or a variant thereof),
an HCDR2 comprising the amino acid sequence set forth in SEQ ID NO: 234 (or a variant thereof), and
an HCDR3 comprising the amino acid sequence set forth in SEQ ID NO: 235 (or a variant thereof); and
a LCVR that comprises:
an LCDR1 comprising the amino acid sequence set forth in SEQ ID NO: 238 (or a variant thereof),
an LCDR2 comprising the amino acid sequence set forth in SEQ ID NO: 239 (or a variant thereof), and
an LCDR3 comprising the amino acid sequence set forth in SEQ ID NO: 240 (or a variant thereof);
(y) a HCVR that comprises:
an HCDR1 comprising the amino acid sequence set forth in SEQ ID NO: 243 (or a variant thereof),
an HCDR2 comprising the amino acid sequence set forth in SEQ ID NO: 244 (or a variant thereof), and
an HCDR3 comprising the amino acid sequence set forth in SEQ ID NO: 245 (or a variant thereof); and
a LCVR that comprises:
an LCDR1 comprising the amino acid sequence set forth in SEQ ID NO: 248 (or a variant thereof),
an LCDR2 comprising the amino acid sequence set forth in SEQ ID NO: 249 (or a variant thereof), and
an LCDR3 comprising the amino acid sequence set forth in SEQ ID NO: 250 (or a variant thereof);
(z) a HCVR that comprises:
an HCDR1 comprising the amino acid sequence set forth in SEQ ID NO: 253 (or a variant thereof),
an HCDR2 comprising the amino acid sequence set forth in SEQ ID NO: 254 (or a variant thereof), and
an HCDR3 comprising the amino acid sequence set forth in SEQ ID NO: 255 (or a variant thereof); and
a LCVR that comprises:
an LCDR1 comprising the amino acid sequence set forth in SEQ ID NO: 258 (or a variant thereof),
an LCDR2 comprising the amino acid sequence set forth in SEQ ID NO: 259 (or a variant thereof), and
an LCDR3 comprising the amino acid sequence set forth in SEQ ID NO: 260 (or a variant thereof); (aa) a HCVR that comprises:
an HCDR1 comprising the amino acid sequence set forth in SEQ ID NO: 263 (or a variant thereof),
an HCDR2 comprising the amino acid sequence set forth in SEQ ID NO: 264 (or a variant thereof), and
an HCDR3 comprising the amino acid sequence set forth in SEQ ID NO: 265 (or a variant thereof); and
a LCVR that comprises:
an LCDR1 comprising the amino acid sequence set forth in SEQ ID NO: 268 (or a variant thereof),
an LCDR2 comprising the amino acid sequence set forth in SEQ ID NO: 269 (or a variant thereof), and
an LCDR3 comprising the amino acid sequence set forth in SEQ ID NO: 270 (or a variant thereof); (ab) a HCVR that comprises:
an HCDR1 comprising the amino acid sequence set forth in SEQ ID NO: 273 (or a variant thereof),
an HCDR2 comprising the amino acid sequence set forth in SEQ ID NO: 274 (or a variant thereof), and
an HCDR3 comprising the amino acid sequence set forth in SEQ ID NO: 275 (or a variant thereof); and
a LCVR that comprises:
an LCDR1 comprising the amino acid sequence set forth in SEQ ID NO: 278 (or a variant thereof),
an LCDR2 comprising the amino acid sequence set forth in SEQ ID NO: 279 (or a variant thereof), and
an LCDR3 comprising the amino acid sequence set forth in SEQ ID NO: 280 (or a variant thereof); (ac) a HCVR that comprises:
an HCDR1 comprising the amino acid sequence set forth in SEQ ID NO: 283 (or a variant thereof),
an HCDR2 comprising the amino acid sequence set forth in SEQ ID NO: 284 (or a variant thereof), and
an HCDR3 comprising the amino acid sequence set forth in SEQ ID NO: 285 (or a variant thereof); and
a LCVR that comprises:
an LCDR1 comprising the amino acid sequence set forth in SEQ ID NO: 288 (or a variant thereof),
an LCDR2 comprising the amino acid sequence set forth in SEQ ID NO: 289 (or a variant thereof), and
an LCDR3 comprising the amino acid sequence set forth in SEQ ID NO: 290 (or a variant thereof); (ad) a HCVR that comprises:
an HCDR1 comprising the amino acid sequence set forth in SEQ ID NO: 293 (or a variant thereof),
an HCDR2 comprising the amino acid sequence set forth in SEQ ID NO: 294 (or a variant thereof), and
an HCDR3 comprising the amino acid sequence set forth in SEQ ID NO: 295 (or a variant thereof); and
a LCVR that comprises:
an LCDR1 comprising the amino acid sequence set forth in SEQ ID NO: 298 (or a variant thereof),
an LCDR2 comprising the amino acid sequence set forth in SEQ ID NO: 299 (or a variant thereof), and
an LCDR3 comprising the amino acid sequence set forth in SEQ ID NO: 300 (or a variant thereof);
(ae) a HCVR that comprises:
an HCDR1 comprising the amino acid sequence set forth in SEQ ID NO: 303 (or a variant thereof),
an HCDR2 comprising the amino acid sequence set forth in SEQ ID NO: 304 (or a variant thereof), and
an HCDR3 comprising the amino acid sequence set forth in SEQ ID NO: 305 (or a variant thereof); and
a LCVR that comprises:
an LCDR1 comprising the amino acid sequence set forth in SEQ ID NO: 308 (or a variant thereof),
an LCDR2 comprising the amino acid sequence set forth in SEQ ID NO: 309 (or a variant thereof), and
an LCDR3 comprising the amino acid sequence set forth in SEQ ID NO: 310 (or a variant thereof);
and/or
(af) a HCVR that comprises:
an HCDR1 comprising the amino acid sequence set forth in SEQ ID NO: 313 (or a variant thereof),
an HCDR2 comprising the amino acid sequence set forth in SEQ ID NO: 314 (or a variant thereof), and
an HCDR3 comprising the amino acid sequence set forth in SEQ ID NO: 315 (or a variant thereof); and
a LCVR that comprises:
an LCDR1 comprising the amino acid sequence set forth in SEQ ID NO: 318 (or a variant thereof),
an LCDR2 comprising the amino acid sequence set forth in SEQ ID NO: 319 (or a variant thereof), and
an LCDR3 comprising the amino acid sequence set forth in SEQ ID NO: 320 (or a variant thereof);
or the scFv comprises:
(i) a HCVR that comprises the amino acid sequence set forth in SEQ ID NO: 2 or 462 (or a variant thereof); and a LCVR that comprises the amino acid sequence set forth in SEQ ID NO: 7 (or a variant thereof);
(ii) a HCVR that comprises the amino acid sequence set forth in SEQ ID NO: 12 or 463 (or a variant thereof); and a LCVR that comprises the amino acid sequence set forth in SEQ ID NO: 17 (or a variant thereof);
(iii) a HCVR that comprises the amino acid sequence set forth in SEQ ID NO: 22 or 464 (or a variant thereof); and a LCVR that comprises the amino acid sequence set forth in SEQ ID NO: 27 (or a variant thereof);
(iv) a HCVR that comprises the amino acid sequence set forth in SEQ ID NO: 32 (or a variant thereof); and a LCVR that comprises the amino acid sequence set forth in SEQ ID NO: 37 or 465 (or a variant thereof);
(v) a HCVR that comprises the amino acid sequence set forth in SEQ ID NO: 42 (or a variant thereof); and a LCVR that comprises the amino acid sequence set forth in SEQ ID NO: 47 or 466 (or a variant thereof);
(vi) a HCVR that comprises the amino acid sequence set forth in SEQ ID NO: 52 or 467 (or a variant thereof); and a LCVR that comprises the amino acid sequence set forth in SEQ ID NO: 57 or 468 (or a variant thereof);
(vii) a HCVR that comprises the amino acid sequence set forth in SEQ ID NO: 62 or 492 (or a variant thereof); and a LCVR that comprises the amino acid sequence set forth in SEQ ID NO: 67 or 469 (or a variant thereof);
(viii) a HCVR that comprises the amino acid sequence set forth in SEQ ID NO: 72 or 470 (or a variant thereof); and a LCVR that comprises the amino acid sequence set forth in SEQ ID NO: 77 or 471 (or a variant thereof);
(ix) a HCVR that comprises the amino acid sequence set forth in SEQ ID NO: 82 (or a variant thereof); and a LCVR that comprises the amino acid sequence set forth in SEQ ID NO: 87 (or a variant thereof);
(x) a HCVR that comprises the amino acid sequence set forth in SEQ ID NO: 92 or 472 (or a variant thereof); and a LCVR that comprises the amino acid sequence set forth in SEQ ID NO: 97 (or a variant thereof);
(xi) a HCVR that comprises the amino acid sequence set forth in SEQ ID NO: 102 (or a variant thereof); and a LCVR that comprises the amino acid sequence set forth in SEQ ID NO: 107 (or a variant thereof);
(xii) a HCVR that comprises the amino acid sequence set forth in SEQ ID NO: 112 or 473 (or a variant thereof); and a LCVR that comprises the amino acid sequence set forth in SEQ ID NO: 117 or 474 (or a variant thereof);
(xiii) a HCVR that comprises the amino acid sequence set forth in SEQ ID NO: 122 (or a variant thereof); and a LCVR that comprises the amino acid sequence set forth in SEQ ID NO: 127 (or a variant thereof);
(xiv) a HCVR that comprises the amino acid sequence set forth in SEQ ID NO: 132 (or a variant thereof); and a LCVR that comprises the amino acid sequence set forth in SEQ ID NO: 137 (or a variant thereof);
(xv) a HCVR that comprises the amino acid sequence set forth in SEQ ID NO: 142 or 475 (or a variant thereof); and a LCVR that comprises the amino acid sequence set forth in SEQ ID NO: 147 or 476 (or a variant thereof);
(xvi) a HCVR that comprises the amino acid sequence set forth in SEQ ID NO: 152 (or a variant thereof); and a LCVR that comprises the amino acid sequence set forth in SEQ ID NO: 157 (or a variant thereof);
(xvii) a HCVR that comprises the amino acid sequence set forth in SEQ ID NO: 162 or 477 (or a variant thereof); and a LCVR that comprises the amino acid sequence set forth in SEQ ID NO: 167 (or a variant thereof);
(xviii) a HCVR that comprises the amino acid sequence set forth in SEQ ID NO: 172 (or a variant thereof); and a LCVR that comprises the amino acid sequence set forth in SEQ ID NO: 177 (or a variant thereof);
(xix) a HCVR that comprises the amino acid sequence set forth in SEQ ID NO: 182 or 478 (or a variant thereof); and a LCVR that comprises the amino acid sequence set forth in SEQ ID NO: 187 or 479 (or a variant thereof);
(xx) a HCVR that comprises the amino acid sequence set forth in SEQ ID NO: 192 or 480 (or a variant thereof); and a LCVR that comprises the amino acid sequence set forth in SEQ ID NO: 197 (or a variant thereof);
(xxi) a HCVR that comprises the amino acid sequence set forth in SEQ ID NO: 202 or 481 (or a variant thereof); and a LCVR that comprises the amino acid sequence set forth in SEQ ID NO: 207 (or a variant thereof);
(xxii) a HCVR that comprises the amino acid sequence set forth in SEQ ID NO: 212 (or a variant thereof); and a LCVR that comprises the amino acid sequence set forth in SEQ ID NO: 217 (or a variant thereof);
(xxiii) a HCVR that comprises the amino acid sequence set forth in SEQ ID NO: 222 (or a variant thereof); and a LCVR that comprises the amino acid sequence set forth in SEQ ID NO: 227 (or a variant thereof);
(xxiv) a HCVR that comprises the amino acid sequence set forth in SEQ ID NO: 232 (or a variant thereof); and a LCVR that comprises the amino acid sequence set forth in SEQ ID NO: 237 (or a variant thereof);
(xxv) a HCVR that comprises the amino acid sequence set forth in SEQ ID NO: 242 (or a variant thereof); and a LCVR that comprises the amino acid sequence set forth in SEQ ID NO: 247 (or a variant thereof);
(xxvi) a HCVR that comprises the amino acid sequence set forth in SEQ ID NO: 252 or 482 (or a variant thereof); and a LCVR that comprises the amino acid sequence set forth in SEQ ID NO: 257 (or a variant thereof);
(xxvii) a HCVR that comprises the amino acid sequence set forth in SEQ ID NO: 262 (or a variant thereof); and a LCVR that comprises the amino acid sequence set forth in SEQ ID NO: 267 (or a variant thereof);
(xxviii) a HCVR that comprises the amino acid sequence set forth in SEQ ID NO: 272 (or a variant thereof); and a LCVR that comprises the amino acid sequence set forth in SEQ ID NO: 277 (or a variant thereof);
(xxix) a HCVR that comprises the amino acid sequence set forth in SEQ ID NO: 282 (or a variant thereof); and a LCVR that comprises the amino acid sequence set forth in SEQ ID NO: 287 (or a variant thereof);
(xxx) a HCVR that comprises the amino acid sequence set forth in SEQ ID NO: 292 (or a variant thereof); and a LCVR that comprises the amino acid sequence set forth in SEQ ID NO: 297 (or a variant thereof);
(xxxi) a HCVR that comprises the amino acid sequence set forth in SEQ ID NO: 302 or 483 (or a variant thereof); and a LCVR that comprises the amino acid sequence set forth in SEQ ID NO: 307 or 527 (or a variant thereof); and/or
(xxxii) a HCVR that comprises the amino acid sequence set forth in SEQ ID NO: 312 (or a variant thereof); and a LCVR that comprises the amino acid sequence set forth in SEQ ID NO: 317 or 484 (or a variant thereof);
e.g., wherein the HCVR and LCVR are in either orientation (HCVR-LCVR or LCVR-HCVR),
optionally, wherein the HCVR and LCVR are linked by a linker, e.g., that comprises an amino acid sequence, e.g., about 10 amino acids in length, for example: (Gly₄Ser)ₙ (SEQ ID NO: 426) wherein n is 1, 2, 3, 4, 5, 6, 7, 8, 9 or 10.

In some embodiments, an anti-hTfR scFv described herein, in V_{L}-(Gly₄Ser)₃ (SEQ ID NO: 541)-V_{H} format, comprises an amino acid sequence as set forth in Table 1-2. In other embodiments, scFvs described herein may be in the format V_{H}-(Gly₄Ser)₃ (SEQ ID NO: 541)-V_{L}. Optionally, an anti-hTfR scFv described herein further includes an N-terminal LLQGSG (SEQ ID NO: 452) and/or a C-terminal HHHHHH (SEQ ID NO: 501).

TABLE 1-2
Anti-hTfR scFv Molecules in Described Protein-drug Conjugates.

| | SEQ | |
|---|---|---|
| Antibody | ID | |
| clone | NO | Amino acid sequence (Vk-3xG4S(SEQ ID NO: 541)-Vh) |
| 12795B | 392 | DIQMTQSPSSLSASVGDRVTITCRASQGIRDHFGWYQQKPGKAPKRLIYAA |
| | | SSLHSGVPSRFSGSGSGTEFTLTISSLQPEDFATYYCLQYDTYPLTFGGGTK |
| | | VEIKGGGGSGGGGSGGGGSEVQLVESGGGLVQPGGSLRLSCATSGFTFTS |
| | | YDMKWVRQAPGLGLEWVSAISGSGGNTYYADSVKGRFTISRDNSRNTLYL |
| | | QMNSLRAEDTAVYYCTRSHDFGAFDYFDYWGQGTLVTVSS |
| 12798B | 393 | EIVMTQSPATLSVSPGERATLSCRASQTVSSNLAWYQQKPGQAPRLLIYGS |
| | | SSRATGIPARFSGSGSGTEFTLTISSLQSEDFAVYYCQQYNNWPPYTFGQG |
| | | TKLEIKGGGGSGGGGSGGGGSEVQLVESGGDLVQPGRSLRLSCAASGFTF |
| | | DDYAMHWVRQAPGKGLEWVSGISWNSATRVYADSVKGRFTISRDNAKNFL |
| | | YLQMNSLRSEDTALYHCAKDMDISLGYYGLDVWGQGTTVTVSS |
| 12799B | 394 | DIQMTQSPSSVSASVGDRVTITCRASQGIASWLAWYQQKPGKAPELLIYAAS |
| | | SLQGGVPSRFSGSGSGTDFTLTISSLQPEDFAIYYCQQANYFPWTFGQGTK |
| | | VEIKGGGGSGGGGSGGGGSQITLKESGPTLVKPTQTLTLTCTFSGFSLSTS |
| | | GVGVVWIRQPPGKALEWLALIYWNDHKRYSPSLGSRLTITKDTSKNQVVLT |
| | | MTNMDPVDTATYYCAHYSGSYSYYYYGLDVWGQGTTVTVSS |
| 12801B | 395 | DIQMTQSPSSLSASVGDRVTITCRASQGIRTDLGWYQQKPGKAPKRLIYAAS |
| | | SLQSGVPSRFSGSGSGTEFTLTISSLRPEDFATFYCLQYNSYPLTFGGGTKV |
| | | EIKGGGGSGGGGSGGGGSEVQLLESGGALVQPGGSLRLSCAASGFTFTSY |
| | | AMHWVRQAPGKGLEWVSSIRGSGGGTYSADSVKGRFTISRDNSRDTLYLQ |
| | | MNSVRAEDTAVYYCARSHDYGAFDFFDYWGQGTLVTVSS |
| 12802B | 396 | EIVMTQSPATLSVSPGERATLSCRASQSVSINLAWYQQKPGQAPRLLIFVAS |
| | | TRATGIPARFSGSGSGTEFTLTISSLQSEDFATYYCQQYDIWPYTFGQGTKL |
| | | EIKGGGGSGGGGSGGGGSQVQLVESGGGLVKPGGSLRLSCAASGFTFSD |
| | | YFMSWIRQAPGKGLEWVSYISSTGSTINYADSVKGRFTISRDNVKNSLYLQM |
| | | TSLRVEDTAVYYCTRDNWNYEYWGQGTLVTVSS |
| 12808B | 397 | DIQMTQSPSSLSASVGDRVTINCRASQGIRNDLGWYQQKPGKAPKRLIYAA |
| | | SSLQSGVPLRFSGSGSGTEFTLTINNLQPEDFATYYCLSHNSYPWTFGQGT |
| | | KVEIKGGGGSGGGGSGGGGSQLQLQESGPGLVKPSETLSLTCTVSGESISS |
| | | NTYYWGWIRQPPGKGLEWIGSIDYSGTTNYNPSLKSRVTISVDTSRNHFSLR |
| | | LRSVTAADTAVYYCAREWGNYGYYYGMDVWGQGTTVTVSS |
| 12812B | 398 | DIQMTQSPPSVSASVGDRVTITCRASQGISSWLAWYQQKPGKAPKLLIYAAS |
| | | SLQSGVPSRFSGSGSGTDFTLTISSLQPEDFATYYCQQANSFPRTFGQGTK |
| | | VEIKGGGGSGGGGSGGGGSQVQLVQSGAEVKKPGSSVRVSCKASRGTFS |
| | | SYAISWVRQAPGQGLEWMGGIIPIFGTANYAQKFLARVTITADESTSTAYME |
| | | LSSLRSEDTAVYYCAREKGWNYFDYWGQGTLVTVSS |
| 12816B | 399 | DIVMTQSPLSLPVTPGEPASISCRSSQSLLHGNGYNYLTWYLQKPGQSPQL |
| | | LIYLGSNRASGVPDRFSGSGSGTDFTLKISRVEAEDVGVYYCMQALQTPYTF |
| | | GQGTKLEIKGGGGSGGGGSGGGGSQVQLVESGGGLVKPGGSLRLSCAAS |
| | | GFTFSDYYMNWIRQAPGKGLEWVSYISSSGTTIYYADSVKGRFTISRDNAKK |
| | | SLYLEMNSLRAEDTAVYYCAREGYGNDYYYYGIDVWGQGTTVTVSS |
| 12833B | 400 | DIQMTQSPSSLSASVGDRVTITCRASQSISSYLNWYQQKPGKAPKLLIYAAS |
| | | SLQSGVPSRFSGSGSGTDFTLTISSLQPEDFATYYCQQSYSTPPITFGQGTR |
| | | LEIKGGGGSGGGGSGGGGSQVQLVESGGGVVQPGRSLRLSCAASGFTFS |
| | | SFGMHWVRQAPGKGLEWVIFISYDGSDKYYADSVKGRFAISRDSSKNTLYL |
| | | QMNSLRAEDTAVYYCAKENGILTDSYGMDVWGQGTTVTVSS |
| 12834B | 401 | DIQMTQSPSSLSASVGDRVTITCRASQSISSYLNWYQQKPGKAPKLLIYAAS |
| | | SLQSGVPSRFSGSGSGTDFTLTISSLQPEDFATYYCQQSYSTPPITFGQGTR |
| | | LEIKGGGGSGGGGSGGGGSQVQLVQSGAEVKKPGASVKVSCKASGYTFT |
| | | SYGISWVRQAPGQGLEWMGWISVYHGNTNYAQKFQGRVTMTTDTSTSTA |
| | | YMELRSLRSDDTAVYYCAREGYYDFWSGYYPFDYWGQGTLVTVSS |
| 12835B | 402 | DIQMTQSPSSLSASVGDRVTITCRASQSISSYLNWYQQKPGKAPKLLIYAAS |
| | | SLQSGVPSRFSGSGSGTDFTLTISSLQPEDFATYYCQQSYSTPPITFGQGTR |
| | | LEIKGGGGSGGGGSGGGGSEVQLVESGGGLIQPGGSLRLSCEASGFTFRN |
| | | YEMNWVRQAPGKGLEWVSYISSSGNMKDYAESVKGRFTISRDNVKNSLQL |
| | | QMNSLRVEDTAVYYCARDEFPYGMDVWGQGTTVTVSS |
| 12839B | 403 | DIQMTQSPSSLSASVGDRVTITCRASQSISSYLNWYQQKPGKAPKLLIYAAS |
| | | SLQSGVPSRFSGSGSGTDFTLTISSLQPEDFATYYCQQSYSTPPITFGQGTR |
| | | LEIKGGGGSGGGGSGGGGSQVQLVESGGGVVQPGRSLRLSCAASGFPFS |
| | | NYVMYWVRQAPGKGLEWVALIFFDGKKNYHADSVKGRFTITRDNSKNMLYL |
| | | QMNSLRPEDAAVYYCAKIHCPNGVCYKGYYGMDVWGQGTTVTVSS |
| 12841B | 404 | DIQMTQSPSSLSASVGDRVTITCRASQSISSYLNWYQQKPGKAPKLLIYAAS |
| | | SLQSGVPSRFSGSGSGTDFTLTISSLQPEDFATYYCQQSYSTPPITFGQGTR |
| | | LEIKGGGGSGGGGSGGGGSEVQLVESGGGLVQPGGSLRLSCAASGFTFS |
| | | NYWMNWVRQAPGKGLEWVANIKEDGGKKLYVDSVKGRFTISRDNAKNSLF |
| | | LQMNSLRAEDTAVYYCAREDTTLVVDYYYYGMDVWGQGTTVTVSS |
| 12843B | 405 | DIQMTQSPSSLSASVGDRVTITCRASQSISSYLNWYQQKPGKAPKLLIYAAS |
| | | SLQSGVPSRFSGSGSGTDFTLTISSLQPEDFATYYCQQSYSTPPITFGQGTR |
| | | LEIKGGGGSGGGGSGGGGSEVQLVESGGGLVQPGGSLRLSCAASGFTFNI |
| | | FEMNWVRQAPGKGLEWISYISSRGTTTYYADSVRGRFTISRDNAKNSLYLQ |
| | | MNSLRAEDTAVYYCARDYEATIPFDFWGQGTLVTVSS |
| 12844B | 406 | DIQMTQSPSSLSASVGDRVTITCRASQSISSYLNWYQQKPGKAPKLLIYAAS |
| | | SLQSGVPSRFSGSGSGTDFTLTISSLQPEDFATYYCQQSYSTPPITFGQGTR |
| | | LEIKGGGGSGGGGSGGGGSEVQLVESGGSVVRPGGSLRLSCEASGFTFD |
| | | DYGMSWVRQDPGKGLEWVSGINWNGDRTNYADSVKGRFIISRDNAKNSVY |
| | | LQMNSLRAEDSALYHCARDQGLGVAATLDYWGQGTLVTVSS |
| 12845B | 407 | DIQMTQSPSSLSASVGDRVTITCRASQSISSYLNWYQQKPGKAPKLLIYAAS |
| | | SLQSGVPSRFSGSGSGTDFTLTISSLQPEDFATYYCQQSYSTPPITFGQGTR |
| | | LEIKGGGGSGGGGSGGGGSEVQLVESGGGLVQPGGSLRLSCAASGFTVS |
| | | NYEMNWVRQAPGKGLEWVSYISSSTSNIYYADSVKGRFTISRDNAENSLYL |
| | | QMNSLRVEDTAVYYCVRDGIVVVPVGRGYYYYGLDVWGQGTTVTVSS |
| 12847B | 408 | DIQMTQSPSSLSASVGDRVTITCRASQSISSYLNWYQQKPGKAPKLLIYAAS |
| | | SLQSGVPSRFSGSGSGTDFTLTISSLQPEDFATYYCQQSYSTPPITFGQGTR |
| | | LEIKGGGGSGGGGSGGGGSEVQLVESGGGLVQPGRSLRLSCAASGFTFD |
| | | DYAMNWVRQAPGKGLEWVSGISWSSGSMDYADSVKGRFTISRDNAKNSL |
| | | YLQMNSLRTEDTALYYCAKAREVGDYYGMDVWGQGTTVTVSS |
| 12848B | 409 | EIVLTQSPGTLSLSPGERATLSCRASQSVSSSYLAWYQQKPGQAPRLLIYGA |
| | | SSRATGIPDRFSGSGSGTDFTLTISRLEPEDFAVYYCQQYGSSPWTFGQGT |
| | | KVEIKGGGGSGGGGSGGGGSEVQLVESGGGLVQPGRSLTLSCAASGFTFD |
| | | NFGMHWVRQGPGKGLEWVSGLTWNSGVIGYADSVKGRFTISRDNAKNSLY |
| | | LQMNSLRPEDTALYYCAKDIRNYGPFDYWGQGTLVTVSS |
| 12850B | 410 | EIVLTQSPGTLSLSPGERATLSCRASQSVSSSYLAWYQQKPGQAPRLLIYGA |
| | | SSRATGIPDRFSGSGSGTDFTLTISRLEPEDFAVYYCQQYGSSPWTFGQGT |
| | | KVEIKGGGGSGGGGSGGGGSQVQLVQSGAEVKKPGSSVKVSCKASGGTF |
| | | NTYAITWVRQAPGQGLEWMGGIIPISGIAEYAQKFQGRVTITTDDSSTTAYM |
| | | ELNSLRSEDTAVYYCASWNYALYYFYGMDVWGRGTTVTVSS |
| 31863B | 411 | DIQMTQSPSSLSASIGDRVTITCRASQGISNYLAWYQQKPGKVPKLLIYAAST |
| | | LQSGVPSRFSGSGSGTDFTLTISSLQPEDVATYYCQNHNSVPLTFGGGTKV |
| | | EIKGGGGSGGGGSGGGGSEVQLVESGGGLVQPGGSLRLSCAASGFTFNS |
| | | YAMTWVRQAPGKGLEWVSFIGGSTGNTYYAGSVKGRFTISSDNSKKTLYLQ |
| | | MNSLRAEDTAVYYCAKGGAARRMEYFQHWGQGTLVTVSS |
| 31874B | 412 | DIQMTQSPSSLSASVGDRVTITCRASQGISNYLAWYQQKPGKVPNLLIYAAS |
| | | TLQSGVPSRFSGSGSGTDFTLTISSLQPEDVATYYCQKYNSAPLTFGGGTK |
| | | VEIKGGGGSGGGGSGGGGSEVQLVESGGGLVQPGGSLRLSCAASGFAFS |
| | | SYAMTWVRQAPGKGLEWVSVISGTGGSTYYADSVKGRFTISRDNSKNTLYL |
| | | QMNSLRAEDTAVYYCAKGGAARRMEYFQYWGQGTLVTVSS |
| 69261 | 413 | DIVMTQSPLSLPVTPGEPASISCRSSQSLLHSNGYNYLDWYLQKPGQSPQF |
| | | LIYLGSNRASGVPDRFSGSGSGTDFTLKINRVEAEDVGVYYCMQALQTPYT |
| | | FGQGTKLEIKGGGGSGGGGSGGGGSQVQLVESGGGLVKPGGSLRLSCAA |
| | | SGFTFSVYYMNWIRQAPGKGLEWVSYISSSGSTIYYADSVKGRFTISRDNAK |
| | | NSLYLQMNSLRAEDTAVYYCGREGYSGTYSYYGMDVWGQGTTVTVSS |
| 69263 | 414 | DIQMTQSPSSLSASVGDRVTITCRASQDISHYSAWYQQKPGKLPNLLIYAAS |
| | | TLQSGVPSRFSGSGSGTDFSLTTSSLQPEDVATYYCQKYNSVPLTFGGGTK |
| | | VEIKGGGGSGGGGSGGGGSEVQLVESGGGLVQPGRSLRLSCAVSGFTFD |
| | | DYAMHWVRQAPGKGLEWVSGISWNSGTRGYADSVKGRFTISRDNAKNSLY |
| | | LQMNSLRGEDTALYYCVKDITISPNYYGMDVWGQGTTVTVSS |
| 69305 | 415 | DIQMTQSPSSLSASVGDRVTITCRASQSIDRYLNWYRQKPGKAPKLLIYTTS |
| | | SLQSGVPSRFSGSGSGTDFTLTLSSLQPEDFATYYCQQSYSPPLTFGGGTK |
| | | VEIKGGGGSGGGGSGGGGSQVQLVESGGGVVQPGRSLRLSCAASGFTFS |
| | | SYGMHWVRQAPGKGLEWVAVIWYDGSNKYYADSVKGRFTISRDISKNTLYL |
| | | QMNSLRAEDTAVYYCAGQLDLFFDYWGQGTLVTVSS |
| 69307 | 416 | DIQMTQSPSSVSASVGDRVTITCRASQGISSWLAWYQQKPGKAPKLLIYAAS |
| | | SLQSGVPSRFSGSGSGTDFTLTISSLQPEDFATYYCQKADSLPYAFGQGTK |
| | | LEIKGGGGSGGGGSGGGGSEVQLVESGGGLVQPGGSLRLSCTASGFTFSN |
| | | YWMTWVRQAPGKGLEWVANIKEDGSEKEYVDSVKGRFTISRDNAKNSLYL |
| | | QMNSLRGEDTAVYYCARDGEQLVDYYYYYVMDVWGQGTTVTVSS |
| 69323 | 417 | DIQMTQSPSSLSASVGDRVTITCRASQSISSYLNWYQQKPGKAPKVLIYAAS |
| | | SLQSGVPSRFSGSGSGTDFTLTISSLQPEDFATYYCQQSYSIPLTFGGGTKV |
| | | EIKGGGGSGGGGSGGGGSEVQLVESGGGLVQPGRSLRLSCAASGFTFDD |
| | | YAMHWVRQAPGKGLEWVSGISWNSGYIGYADSVKGRFTISRDNAENSLHL |
| | | QMNSLRAEDTALYYCARGGSTLVRGVKGGYYGMDVWGQGTTVTVSS |
| 69326 | 418 | EIVMTQSPATLSVSPGERATLSCRASQSVSSNFAWYQQKPGQAPRLLIYSA |
| | | SSRATGIPVRFSGSGSGTEFTLTISSLQSEDFAVYYCQQYNIWPRTFGQGTK |
| | | VEIKGGGGSGGGGSGGGGSEVQLVESGGGLVQPGGSLRLSCAVSGFIFSS |
| | | YEMNWVRQAPGKGLEWVSYISSSGSTIFYADSVKGRFTISRDNAKNSLYLQ |
| | | MNSLRAEDTAVYYCVSGVVLFDVWGQGTMVTVSS |
| 69329 | 419 | DIQMTQSPSSVSASVGDRVTITCRASQGISSWLAWYQQKPGKAPKLLIYAAS |
| | | SLQSGVPSRFSGSGSGTDFTLTISSLQPEDFATYYCQKANSFPYTFGQGTK |
| | | LEIKGGGGSGGGGSGGGGSEVQLVESGGGLVQPGGSLRLSCAASGFTFS |
| | | NYWMTWVRQAPGKGLEWVANIKEDGSEKDYVDSVKGRFTISRDNAKNSLY |
| | | LQMNSLRGEDTAVYYCARDGEQLVDYYYYYVMDVWGQGTTVTVSS |
| 69331 | 420 | DIQLTQSPSSLSASVGDRVTITCWASQGISSYLAWYQQKPGKAPKLLIYAAS |
| | | TLQSGVPSRFSGSGSGTEFTLTISSLQPEDFATYYCQQLNSYPLTFGGGTKV |
| | | EIKGGGGSGGGGSGGGGSQVQLVESGGGVVQPGRSLRLSCIASGFTFSVY |
| | | GIHWVRQAPGKGLEWMAVISHDGNIKHYADSVKGRFTISRDNSKNTLYLQIN |
| | | SLRTEDTAVYYCAKDTWNSLDTFDIWGQGTMVTVSS |
| 69332 | 421 | AIQMTQSPSSLSASVGDRVTITCRASQGIRNDLGWYQQKPGKAPKLLIYAAS |
| | | TLQSGVPSRFSGSGSGTDFTLTISSLQPEDFATYYCLQDYNYPFTFGPGTKV |
| | | DIKGGGGSGGGGSGGGGSQVTLRESGPALVKPSQTLTLTCTFSGFSLNTY |
| | | GMFVSWIRQPPGKALEWLAHIHWDDDKYYSTSLKTRLTISKDTSKNQVVLT |
| | | MTNMDPVDTATYYCARGHNNLNYIIHWGQGTLVTVSS |
| 69340 | 422 | EIVLTQSPATLSLSPGERATLSCRASQSVSSYLAWYQQKPGQAPRLLIHDVS |
| | | NRATGIPARFSGSGSGTDFTLTISSLEPEDFVVYYCQQRSDWPITFGQGTRL |
| | | EIKGGGGSGGGGSGGGGSEVQLVESGGGLVQPGRSLRLSCAASGFTFDD |
| | | KAMHWVRQVPGKGLEWISGISWNSGTIGYADSVKGRFIISRDNAKNSLYLQ |
| | | MNSLRAEDTALYYCAKDGDTSGWYWYGLDVWGQGTTVTVSS |
| 69348 | 423 | DIQMTQSPSSLSASVGDRVTITCRASQSIRNVLGWFQQKPGKAPQRLIYAAS |
| | | SLQSGVPSRFSGSGSGTEFTLTISSLQPEDFATYYCLQHNFYPLTFGGGTKV |
| | | EIKGGGGSGGGGSGGGGSQVQLVESGGGVVQPGRSLRLSCAASGFTFTT |
| | | YGMHWVRQAPGKGLEWVAVIWYDGSNKYYGDSVKGRFTISRDNSKNTLYL |
| | | QMNSLRVDDTAVYYCTRTHGYTRSSDGFDYWGQGTLVTVSS |

Fab fragments that bind specifically to human transferrin receptor, optionally conjugated to a molecular cargo (anti-TfR Fab-drug conjugates), are provided herein. Fab fragments typically contain one complete light chain, VL and a constant light domain, e.g., kappa (e.g., RTVAAPSVFIFPPSDEQLKSGTASVVCLLNNFYPREAKVQWKVDNALQSGNSQESVTEQDSKDSTYSLS STLTLSKADYEKHKVYACEVTHQGLSSPVTKSFNRGEC (SEQ ID NO: 424)) and the V_{H} and IgG1 CH1 portion (e.g., ASTKGPSVFPLAPSSKSTSGGTAALGCLVKDYFPEPVTVSWNSGALTSGVHTFPAVLQSSGLYSLSSVV TVPSSSLGTQTYICNVNHKPSNTKVDKKVEPKSCDKTH (SEQ ID NO: 425)) or IgG4 CH1 (e.g., ASTKGPSVFPLAPCSRSTSESTAALGCLVKDYFPEPVTVSWNSGALTSGVHTFPAVLQSSGLYSLSSVVTVPSSS LGTKTYTCNVDHKPSNTKVDKRVESKYGPPLLQGSG (SEQ ID NO: 459), or ASTKGPSVFPLAPCSRSTSESTAALGCLVKDYFPEPVTVSWNSGALTSGVHTFPAVLQSSGLYSLSSVVTVPSSS LGTKTYTCNVDHKPSNTKVDKRVESKYGPP (SEQ ID NO: 493)) of one heavy chain. Fab fragment antibodies can be generated by papain digestion of whole IgG antibodies to remove the entire Fc fragment, including the hinge region. For example, Fab proteins described herein may comprise:
(1) a heavy chain variable region (HCVR) that comprises the amino acid sequence set forth in SEQ ID NO: 2 or 462, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 7, or LCDR1, LCDR2 and LCDR3 of such a LCVR-linked to the CL domain;
(2) a heavy chain variable region (HCVR) that comprises the amino acid sequence set forth in SEQ ID NO: 12 or 463, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 17, or LCDR1, LCDR2 and LCDR3 of such a LCVR-linked to the CL domain;
(3) a heavy chain variable region (HCVR) that comprises the amino acid sequence set forth in SEQ ID NO: 22 or 464, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 27, or LCDR1, LCDR2 and LCDR3 of such a LCVR-linked to the CL domain;
(4) a heavy chain variable region (HCVR) that comprises the amino acid sequence set forth in SEQ ID NO: 32, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 37 or 465, or LCDR1, LCDR2 and LCDR3 of such a LCVR-linked to the CL domain;
(5) a heavy chain variable region (HCVR) that comprises the amino acid sequence set forth in SEQ ID NO: 42, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 47 or 466, or LCDR1, LCDR2 and LCDR3 of such a LCVR-linked to the CL domain;
(6) a heavy chain variable region (HCVR) that comprises the amino acid sequence set forth in SEQ ID NO: 52 or 467, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 57 or 468, or LCDR1, LCDR2 and LCDR3 of such a LCVR-linked to the CL domain;
(7) a heavy chain variable region (HCVR) that comprises the amino acid sequence set forth in SEQ ID NO: 62 or 492, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 67 or 469, or LCDR1, LCDR2 and LCDR3 of such a LCVR-linked to the CL domain;
(8) a heavy chain variable region (HCVR) that comprises the amino acid sequence set forth in SEQ ID NO: 72 or 470, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 77 or 471, or LCDR1, LCDR2 and LCDR3 of such a LCVR-linked to the CL domain;
(9) a heavy chain variable region (HCVR) that comprises the amino acid sequence set forth in SEQ ID NO: 82, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 87, or LCDR1, LCDR2 and LCDR3 of such a LCVR-linked to the CL domain;
(10) a heavy chain variable region (HCVR) that comprises the amino acid sequence set forth in SEQ ID NO: 92 or 472, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 97, or LCDR1, LCDR2 and LCDR3 of such a LCVR-linked to the CL domain;
(11) a heavy chain variable region (HCVR) that comprises the amino acid sequence set forth in SEQ ID NO: 102, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 107, or LCDR1, LCDR2 and LCDR3 of such a LCVR-linked to the CL domain;
(12) a heavy chain variable region (HCVR) that comprises the amino acid sequence set forth in SEQ ID NO: 112 or 473, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 117 or 474, or LCDR1, LCDR2 and LCDR3 of such a LCVR-linked to the CL domain;
(13) a heavy chain variable region (HCVR) that comprises the amino acid sequence set forth in SEQ ID NO: 122, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 127, or LCDR1, LCDR2 and LCDR3 of such a LCVR-linked to the CL domain;
(14) a heavy chain variable region (HCVR) that comprises the amino acid sequence set forth in SEQ ID NO: 132, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 137, or LCDR1, LCDR2 and LCDR3 of such a LCVR-linked to the CL domain;
(15) a heavy chain variable region (HCVR) that comprises the amino acid sequence set forth in SEQ ID NO: 142 or 475, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 147 or 476, or LCDR1, LCDR2 and LCDR3 of such a LCVR-linked to the CL domain;
(16) a heavy chain variable region (HCVR) that comprises the amino acid sequence set forth in SEQ ID NO: 152, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 157, or LCDR1, LCDR2 and LCDR3 of such a LCVR-linked to the CL domain;
(17) a heavy chain variable region (HCVR) that comprises the amino acid sequence set forth in SEQ ID NO: 162 or 477, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 167, or LCDR1, LCDR2 and LCDR3 of such a LCVR-linked to the CL domain;
(18) a heavy chain variable region (HCVR) that comprises the amino acid sequence set forth in SEQ ID NO: 172, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 177, or LCDR1, LCDR2 and LCDR3 of such a LCVR-linked to the CL domain;
(19) a heavy chain variable region (HCVR) that comprises the amino acid sequence set forth in SEQ ID NO: 182 or 478, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 187 or 479, or LCDR1, LCDR2 and LCDR3 of such a LCVR-linked to the CL domain;
(20) a heavy chain variable region (HCVR) that comprises the amino acid sequence set forth in SEQ ID NO: 192 or 480, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 197, or LCDR1, LCDR2 and LCDR3 of such a LCVR-linked to the CL domain;
(21) a heavy chain variable region (HCVR) that comprises the amino acid sequence set forth in SEQ ID NO: 202 or 481, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 207, or LCDR1, LCDR2 and LCDR3 of such a LCVR-linked to the CL domain;
(22) a heavy chain variable region (HCVR) that comprises the amino acid sequence set forth in SEQ ID NO: 212, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 217, or LCDR1, LCDR2 and LCDR3 of such a LCVR-linked to the CL domain;
(23) a heavy chain variable region (HCVR) that comprises the amino acid sequence set forth in SEQ ID NO: 222, or a heavy chain variable region that includes HCDR1, HCDR2 and HCDR3 of such a HCVR-linked to the CH1 domain—and comprising a light chain variable region (LCVR) that comprises the amino acid sequence set forth in SEQ ID NO: 227, or LCDR1, LCDR2 and LCDR3 of such a LCVR-linked to the CL domain;
(24) a heavy chain variable region (HCVR) that comprises the amino acid sequence set forth in SEQ ID NO: 232, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 237, or LCDR1, LCDR2 and LCDR3 of such a LCVR-linked to the CL domain;
(25) a heavy chain variable region (HCVR) that comprises the amino acid sequence set forth in SEQ ID NO: 242, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 247, or LCDR1, LCDR2 and LCDR3 of such a LCVR-linked to the CL domain;
(26) a heavy chain variable region (HCVR) that comprises the amino acid sequence set forth in SEQ ID NO: 252 or 482, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 257, or LCDR1, LCDR2 and LCDR3 of such a LCVR-linked to the CL domain;
(27) a heavy chain variable region (HCVR) that comprises the amino acid sequence set forth in SEQ ID NO: 262, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 267, or LCDR1, LCDR2 and LCDR3 of such a LCVR-linked to the CL domain;
(28) a heavy chain variable region (HCVR) that comprises the amino acid sequence set forth in SEQ ID NO: 272, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 277, or LCDR1, LCDR2 and LCDR3 of such a LCVR-linked to the CL domain;
(29) a heavy chain variable region (HCVR) that comprises the amino acid sequence set forth in SEQ ID NO: 282, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 287, or LCDR1, LCDR2 and LCDR3 of such a LCVR-linked to the CL domain;
(30) a heavy chain variable region (HCVR) that comprises the amino acid sequence set forth in SEQ ID NO: 292, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 297, or LCDR1, LCDR2 and LCDR3 of such a LCVR-linked to the CL domain;
(31) a heavy chain variable region (HCVR) that comprises the amino acid sequence set forth in SEQ ID NO: 302 or 483, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 307 or 527, or LCDR1, LCDR2 and LCDR3 of such a LCVR-linked to the CL domain; and/or
(32) a heavy chain variable region (HCVR) that comprises the amino acid sequence set forth in SEQ ID NO: 312, or a heavy chain variable region that includes HCDR1, HCDR2 and HCDR3 of such a HCVR-linked to the CH1 domain—and
a light chain variable region (LCVR) that comprises the amino acid sequence set forth in SEQ ID NO: 317 or 484, or LCDR1, LCDR2 and LCDR3 of such a LCVR-linked to the CL domain;
e.g., wherein CH1 is from IgG1 or IgG4;
e.g., wherein CH1 is SEQ ID NO: 425, 459, or 493.

The heavy and light chains of anti-hTfR Fabs in anti-hTfR protein-drug conjugates described herein are set forth below.

(1) 31874B
Fab Light Chain
(SEQ ID NO: 328)
DIQMTQSPSSLSASVGDRVTITCRASQGISNYLAWYQQKPGKVPNLLIYAASTLQSGVPSRFSGSGSGTDFTLTI
SSLQPEDVATYYCQKYNSAPLTFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 329)
EVQLVESGGGLVQPGGSLRLSCAASGFAFSSYAMTWVRQAPGKGLEWVSVISGTGGSTYYADSVKGRFTISRDNS
KNTLYLQMNSLRAEDTAVYYCAKGGAARRMEYFQYWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCD
KTH
(2) 31863B
Fab Light Chain
(SEQ ID NO: 330)
DIQMTQSPSSLSASIGDRVTITCRASQGISNYLAWYQQKPGKVPKLLIYAASTLQSGVPSRFSGSGSGTDFTLTI
SSLQPEDVATYYCQNHNSVPLTFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 331)
EVQLVESGGGLVQPGGSLRLSCAASGFTFNSYAMTWVRQAPGKGLEWVSFIGGSTGNTYYAGSVKGRFTISSDNS
KKTLYLQMNSLRAEDTAVYYCAKGGAARRMEYFQHWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCD
KTH
(3) 69348
Fab Light Chain
(SEQ ID NO: 332)
DIQMTQSPSSLSASVGDRVTITCRASQSIRNVLGWFQQKPGKAPQRLIYAASSLQSGVPSRFSGSGSGTEFTLTI
SSLOPEDFATYYCLQHNFYPLTFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 333)
QVQLVESGGGVVQPGRSLRLSCAASGFTFTTYGMHWVRQAPGKGLEWVAVIWYDGSNKYYGDSVKGRFTISRDNS
KNTLYLQMNSLRVDDTAVYYCTRTHGYTRSSDGFDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKSC
DKTH
(4) 69340
Fab Light Chain
(SEQ ID NO: 334)
EIVLTQSPATLSLSPGERATLSCRASQSVSSYLAWYQQKPGQAPRLLIHDVSNRATGIPARFSGSGSGTDFTLTI
SSLEPEDFVVYYCQQRSDWPITFGQGTRLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 335)
EVQLVESGGGLVQPGRSLRLSCAASGFTFDDKAMHWVRQVPGKGLEWISGISWNSGTIGYADSVKGRFIISRDNA
KNSLYLQMNSLRAEDTALYYCAKDGDTSGWYWYGLDVWGQGTTVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEPKS
CDKTH
(5) 69331
Fab Light Chain
(SEQ ID NO: 336)
DIQLTQSPSSLSASVGDRVTITCWASQGISSYLAWYQQKPGKAPKLLIYAASTLQSGVPSRFSGSGSGTEFTLTI
SSLQPEDFATYYCQQLNSYPLTFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 337)
QVQLVESGGGVVQPGRSLRLSCIASGFTFSVYGIHWVRQAPGKGLEWMAVISHDGNIKHYADSVKGRFTISRDNS
KNTLYLQINSLRTEDTAVYYCAKDTWNSLDTFDIWGQGTMVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSCDK
TH
(6) 69332
Fab Light Chain
(SEQ ID NO: 338)
AIQMTQSPSSLSASVGDRVTITCRASQGIRNDLGWYQQKPGKAPKLLIYAASTLQSGVPSRFSGSGSGTDFTLTI
SSLQPEDFATYYCLQDYNYPFTFGPGTKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 339)
QVTLRESGPALVKPSQTLTLTCTFSGFSLNTYGMFVSWIRQPPGKALEWLAHIHWDDDKYYSTSLKTRLTISKDT
SKNQVVLTMTNMDPVDTATYYCARGHNNLNYIIHWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSCDK
TH
(7) 69326
Fab Light Chain
(SEQ ID NO: 340)
EIVMTQSPATLSVSPGERATLSCRASQSVSSNFAWYQQKPGQAPRLLIYSASSRATGIPVRFSGSGSGTEFTLTI
SSLQSEDFAVYYCQQYNIWPRTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 341)
EVQLVESGGGLVQPGGSLRLSCAVSGFIFSSYEMNWVRQAPGKGLEWVSYISSSGSTIFYADSVKGRFTISRDNA
KNSLYLQMNSLRAEDTAVYYCVSGVVLFDVWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTH
(8) 69329
Fab Light Chain
(SEQ ID NO: 342)
DIQMTQSPSSVSASVGDRVTITCRASQGISSWLAWYQQKPGKAPKLLIYAASSLQSGVPSRFSGSGSGTDFTLTI
SSLQPEDFATYYCQKANSFPYTFGQGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 343)
EVQLVESGGGLVQPGGSLRLSCAASGFTFSNYWMTWVRQAPGKGLEWVANIKEDGSEKDYVDSVKGRFTISRDNA
KNSLYLQMNSLRGEDTAVYYCARDGEQLVDYYYYYVMDVWGQGTTVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEP
KSCDKTH
(9) 69323
Fab Light Chain
(SEQ ID NO: 344)
DIQMTQSPSSLSASVGDRVTITCRASQSISSYLNWYQQKPGKAPKVLIYAASSLQSGVPSRFSGSGSGTDFTLTI
SSLQPEDFATYYCQQSYSIPLTFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 345)
EVQLVESGGGLVQPGRSLRLSCAASGFTEDDYAMHWVRQAPGKGLEWVSGISWNSGYIGYADSVKGRFTISRDNA
ENSLHLQMNSLRAEDTALYYCARGGSTLVRGVKGGYYGMDVWGQGTTVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKV
EPKSCDKTH
(10) 69305
Fab Light Chain
(SEQ ID NO: 346)
DIQMTQSPSSLSASVGDRVTITCRASQSIDRYLNWYRQKPGKAPKLLIYTTSSLQSGVPSRFSGSGSGTDFTLTL
SSLQPEDFATYYCQQSYSPPLTFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 347)
QVQLVESGGGVVQPGRSLRLSCAASGFTFSSYGMHWVRQAPGKGLEWVAVIWYDGSNKYYADSVKGRFTISRDIS
KNTLYLQMNSLRAEDTAVYYCAGQLDLFFDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTH
(11) 69307
Fab Light Chain
(SEQ ID NO: 348)
DIQMTQSPSSVSASVGDRVTITCRASQGISSWLAWYQQKPGKAPKLLIYAASSLQSGVPSRESGSGSGTDETLTI
SSLQPEDFATYYCQKADSLPYAFGQGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 349)
EVQLVESGGGLVQPGGSLRLSCTASGFTFSNYWMTWVRQAPGKGLEWVANIKEDGSEKEYVDSVKGRFTISRDNA
KNSLYLQMNSLRGEDTAVYYCARDGEQLVDYYYYYVMDVWGQGTTVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEP
KSCDKTH
(12) 12795B
Fab Light Chain
(SEQ ID NO: 350)
DIQMTQSPSSLSASVGDRVTITCRASQGIRDHFGWYQQKPGKAPKRLIYAASSLHSGVPSRFSGSGSGTEFTLTI
SSLQPEDFATYYCLQYDTYPLTFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 351)
EVQLVESGGGLVQPGGSLRLSCATSGFTFTSYDMKWVRQAPGLGLEWVSAISGSGGNTYYADSVKGRFTISRDNS
RNTLYLQMNSLRAEDTAVYYCTRSHDFGAFDYFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCD
KTH
(13) 12798B
Fab Light Chain
(SEQ ID NO: 352)
EIVMTQSPATLSVSPGERATLSCRASQTVSSNLAWYQQKPGQAPRLLIYGSSSRATGIPARFSGSGSGTEFTLTI
SSLQSEDFAVYYCQQYNNWPPYTFGQGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
Fab Heavy Chain
(SEQ ID NO: 353)
EVQLVESGGDLVQPGRSLRLSCAASGFTFDDYAMHWVRQAPGKGLEWVSGISWNSATRVYADSVKGRFTISRDNA
KNFLYLQMNSLRSEDTALYHCAKDMDISLGYYGLDVWGQGTTVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKSC
DKTH;
(SEQ ID NO: 485)
EVQLVESGGDLVQPGRSLRLSCAASGFTFDDYAMHWVRQAPGKGLEWVSGISWNSATRVYADSVKGRFTISRDNA
KNFLYLQMNSLRSEDTALYHCAKDMDISLGYYGLDVWGQGTTVTVSSASTKGPSVFPLAPCSRSTSESTAALGCL
VKDYFPEPVTVSWNSGALTSGVHTFPAVLQSSGLYSLSSVVTVPSSSLGTKTYTCNVDHKPSNTKVDKRVESKYG
PPLLQGSG;
or
(SEQ ID NO: 494)
EVQLVESGGDLVQPGRSLRLSCAASGFTFDDYAMHWVRQAPGKGLEWVSGISWNSATRVYADSVKGRFTISRDNA
KNFLYLQMNSLRSEDTALYHCAKDMDISLGYYGLDVWGQGTTVTVSSASTKGPSVFPLAPCSRSTSESTAALGCL
VKDYFPEPVTVSWNSGALTSGVHTFPAVLQSSGLYSLSSVVTVPSSSLGTKTYTCNVDHKPSNTKVDKRVESKYG
PP
(14) 12799B
Fab Light Chain
(SEQ ID NO: 354)
DIQMTQSPSSVSASVGDRVTITCRASQGIASWLAWYQQKPGKAPELLIYAASSLQGGVPSRFSGSGSGTDFTLTI
SSLQPEDFAIYYCQQANYFPWTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 355)
QITLKESGPTLVKPTQTLTLTCTFSGFSLSTSGVGVVWIRQPPGKALEWLALIYWNDHKRYSPSLGSRLTITKDT
SKNQVVLTMTNMDPVDTATYYCAHYSGSYSYYYYGLDVWGQGTTVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVEPK
SCDKTH;
(SEQ ID NO: 486)
QITLKESGPTLVKPTQTLTLTCTFSGFSLSTSGVGVVWIRQPPGKALEWLALIYWNDHKRYSPSLGSRLTITKDT
SKNQVVLTMTNMDPVDTATYYCAHYSGSYSYYYYGLDVWGQGTTVTVSSASTKGPSVFPLAPCSRSTSESTAALG
CLVKDYFPEPVTVSWNSGALTSGVHTFPAVLQSSGLYSLSSVVTVPSSSLGTKTYTCNVDHKPSNTKVDKRVESK
YGPPLLQGSG;
or
(SEQ ID NO: 495)
QITLKESGPTLVKPTQTLTLTCTFSGFSLSTSGVGVVWIRQPPGKALEWLALIYWNDHKRYSPSLGSRLTITKDT
SKNQVVLTMTNMDPVDTATYYCAHYSGSYSYYYYGLDVWGQGTTVTVSSASTKGPSVFPLAPCSRSTSESTAALG
CLVKDYFPEPVTVSWNSGALTSGVHTFPAVLQSSGLYSLSSVVTVPSSSLGTKTYTCNVDHKPSNTKVDKRVESK
YGPP
(15) 12801B
Fab Light Chain
(SEQ ID NO: 356)
DIQMTQSPSSLSASVGDRVTITCRASQGIRTDLGWYQQKPGKAPKRLIYAASSLQSGVPSRFSGSGSGTEFTLTI
SSLRPEDFATFYCLQYNSYPLTFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 357)
EVQLLESGGALVQPGGSLRLSCAASGFTFTSYAMHWVRQAPGKGLEWVSSIRGSGGGTYSADSVKGRFTISRDNS
RDTLYLQMNSVRAEDTAVYYCARSHDYGAFDFFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCD
KTH
(16) 12802B
Fab Light Chain
(SEQ ID NO: 358)
EIVMTQSPATLSVSPGERATLSCRASQSVSINLAWYQQKPGQAPRLLIFVASTRATGIPARFSGSGSGTEFTLTI
SSLQSEDFATYYCQQYDIWPYTFGQGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 359)
QVQLVESGGGLVKPGGSLRLSCAASGFTFSDYFMSWIRQAPGKGLEWVSYISSTGSTINYADSVKGRFTISRDNV
KNSLYLQMTSLRVEDTAVYYCTRDNWNYEYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTH
(17) 12808B
Fab Light Chain
(SEQ ID NO: 360)
DIQMTQSPSSLSASVGDRVTINCRASQGIRNDLGWYQQKPGKAPKRLIYAASSLQSGVPLRFSGSGSGTEFTLTI
NNLQPEDFATYYCLSHNSYPWTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 361)
QLQLQESGPGLVKPSETLSLTCTVSGESISSNTYYWGWIRQPPGKGLEWIGSIDYSGTTNYNPSLKSRVTISVDT
SRNHFSLRLRSVTAADTAVYYCAREWGNYGYYYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEPKS
CDKTH
(18) 12812B
Fab Light Chain
(SEQ ID NO: 362)
DIQMTQSPPSVSASVGDRVTITCRASQGISSWLAWYQQKPGKAPKLLIYAASSLQSGVPSRFSGSGSGTDFTLTI
SSLQPEDFATYYCQQANSFPRTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 363)
QVQLVQSGAEVKKPGSSVRVSCKASRGTFSSYAISWVRQAPGQGLEWMGGIIPIFGTANYAQKFLARVTITADES
TSTAYMELSSLRSEDTAVYYCAREKGWNYFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
(19) 12816B
Fab Light Chain
(SEQ ID NO: 364)
DIVMTQSPLSLPVTPGEPASISCRSSQSLLHGNGYNYLTWYLQKPGQSPQLLIYLGSNRASGVPDRESGSGSGTD
FTLKISRVEAEDVGVYYCMQALQTPYTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSENRGEC
Fab Heavy Chain
(SEQ ID NO: 365)
QVQLVESGGGLVKPGGSLRLSCAASGFTFSDYYMNWIRQAPGKGLEWVSYISSSGTTIYYADSVKGRFTISRDNA
KKSLYLEMNSLRAEDTAVYYCAREGYGNDYYYYGIDVWGQGTTVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEPKS
CDKTH
(20) 12833B
Fab Light Chain
(SEQ ID NO: 366)
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
Fab Heavy Chain
(SEQ ID NO: 367)
QVQLVESGGGVVQPGRSLRLSCAASGFTFSSFGMHWVRQAPGKGLEWVIFISYDGSDKYYADSVKGRFAISRDSS
KNTLYLQMNSLRAEDTAVYYCAKENGILTDSYGMDVWGQGTTVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKSC
DKTH
(21) 12834B
Fab Light Chain
(SEQ ID NO: 368)
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
Fab Heavy Chain
(SEQ ID NO: 369)
QVQLVQSGAEVKKPGASVKVSCKASGYTFTSYGISWVRQAPGQGLEWMGWISVYHGNTNYAQKFQGRVTMTTDTS
TSTAYMELRSLRSDDTAVYYCAREGYYDFWSGYYPFDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVEPK
SCDKTH
(22) 12835B
Fab Light Chain
(SEQ ID NO: 370)
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
Fab Heavy Chain
(SEQ ID NO: 371)
EVQLVESGGGLIQPGGSLRLSCEASGFTFRNYEMNWVRQAPGKGLEWVSYISSSGNMKDYAESVKGRFTISRDNV
KNSLQLQMNSLRVEDTAVYYCARDEFPYGMDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
(23) 12847B
Fab Light Chain
(SEQ ID NO: 372)
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
Fab Heavy Chain
(SEQ ID NO: 373)
EVQLVESGGGLVQPGRSLRLSCAASGFTFDDYAMNWVRQAPGKGLEWVSGISWSSGSMDYADSVKGRFTISRDNA
KNSLYLQMNSLRTEDTALYYCAKAREVGDYYGMDVWGQGTTVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCD
KTH;
(SEQ ID NO: 487)
EVQLVESGGGLVQPGRSLRLSCAASGFTEDDYAMNWVRQAPGKGLEWVSGISWSSGSMDYADSVKGRFTISRDNA
KNSLYLQMNSLRTEDTALYYCAKAREVGDYYGMDVWGQGTTVTVSSASTKGPSVFPLAPCSRSTSESTAALGCLV
KDYFPEPVTVSWNSGALTSGVHTFPAVLQSSGLYSLSSVVTVPSSSLGTKTYTCNVDHKPSNTKVDKRVESKYGP
PLLQGSG;
or
(SEQ ID NO: 496)
EVQLVESGGGLVQPGRSLRLSCAASGFTFDDYAMNWVRQAPGKGLEWVSGISWSSGSMDYADSVKGRFTISRDNA
KNSLYLQMNSLRTEDTALYYCAKAREVGDYYGMDVWGQGTTVTVSSASTKGPSVFPLAPCSRSTSESTAALGCLV
KDYFPEPVTVSWNSGALTSGVHTFPAVLQSSGLYSLSSVVTVPSSSLGTKTYTCNVDHKPSNTKVDKRVESKYGP
P
(24) 12848B
Fab Light Chain
(SEQ ID NO: 374)
EIVLTQSPGTLSLSPGERATLSCRASQSVSSSYLAWYQQKPGQAPRLLIYGASSRATGIPDRFSGSGSGTDFTLT
ISRLEPEDFAVYYCQQYGSSPWTFGQGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
Fab Heavy Chain
(SEQ ID NO: 375)
EVQLVESGGGLVQPGRSLTLSCAASGFTFDNFGMHWVRQGPGKGLEWVSGLTWNSGVIGYADSVKGRFTISRDNA
KNSLYLQMNSLRPEDTALYYCAKDIRNYGPFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
H
(25) 12843B
Fab Light Chain
(SEQ ID NO: 376)
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
Fab Heavy Chain
(SEQ ID NO: 377)
EVQLVESGGGLVQPGGSLRLSCAASGFTFNIFEMNWVRQAPGKGLEWISYISSRGTTTYYADSVRGRFTISRDNA
KNSLYLQMNSLRAEDTAVYYCARDYEATIPFDFWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
H;
(SEQ ID NO: 488)
EVQLVESGGGLVQPGGSLRLSCAASGFTENIFEMNWVRQAPGKGLEWISYISSRGTTTYYADSVRGRFTISRDNA
KNSLYLQMNSLRAEDTAVYYCARDYEATIPFDFWGQGTLVTVSSASTKGPSVFPLAPCSRSTSESTAALGCLVKD
YFPEPVTVSWNSGALTSGVHTFPAVLQSSGLYSLSSVVTVPSSSLGTKTYTCNVDHKPSNTKVDKRVESKYGPPL
LQGSG;
or
(SEQ ID NO: 497)
EVQLVESGGGLVQPGGSLRLSCAASGFTFNIFEMNWVRQAPGKGLEWISYISSRGTTTYYADSVRGRFTISRDNA
KNSLYLQMNSLRAEDTAVYYCARDYEATIPFDFWGQGTLVTVSSASTKGPSVFPLAPCSRSTSESTAALGCLVKD
YFPEPVTVSWNSGALTSGVHTFPAVLQSSGLYSLSSVVTVPSSSLGTKTYTCNVDHKPSNTKVDKRVESKYGPP
(26) 12844B
Fab Light Chain
(SEQ ID NO: 378)
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
Fab Heavy Chain
(SEQ ID NO: 379)
EVQLVESGGSVVRPGGSLRLSCEASGFTEDDYGMSWVRQDPGKGLEWVSGINWNGDRTNYADSVKGRFIISRDNA
KNSVYLQMNSLRAEDSALYHCARDQGLGVAATLDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCD
KTH
(27) 12845B
Fab Light Chain
(SEQ ID NO: 380)
DIQMTQSPSSLSASVGDRVTITCRASQSISSYLNWYQQKPGKAPKLLIYAASSLOSGVPSRFSGSGSGTDFTLTI
SSLQPEDFATYYCQQSYSTPPITFGQGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
Fab Heavy Chain
(SEQ ID NO: 381)
EVQLVESGGGLVQPGGSLRLSCAASGFTVSNYEMNWVRQAPGKGLEWVSYISSSTSNIYYADSVKGRFTISRDNA
ENSLYLQMNSLRVEDTAVYYCVRDGIVVVPVGRGYYYYGLDVWGQGTTVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKK
VEPKSCDKTH;
(SEQ ID NO: 489)
EVQLVESGGGLVQPGGSLRLSCAASGFTVSNYEMNWVRQAPGKGLEWVSYISSSTSNIYYADSVKGRFTISRDNA
ENSLYLQMNSLRVEDTAVYYCVRDGIVVVPVGRGYYYYGLDVWGQGTTVTVSSASTKGPSVFPLAPCSRSTSEST
AALGCLVKDYFPEPVTVSWNSGALTSGVHTFPAVLQSSGLYSLSSVVTVPSSSLGTKTYTCNVDHKPSNTKVDKR
VESKYGPPLLQGSG;
or
(SEQ ID NO: 498)
EVQLVESGGGLVQPGGSLRLSCAASGFTVSNYEMNWVRQAPGKGLEWVSYISSSTSNIYYADSVKGRFTISRDNA
ENSLYLQMNSLRVEDTAVYYCVRDGIVVVPVGRGYYYYGLDVWGQGTTVTVSSASTKGPSVFPLAPCSRSTSEST
AALGCLVKDYFPEPVTVSWNSGALTSGVHTFPAVLQSSGLYSLSSVVTVPSSSLGTKTYTCNVDHKPSNTKVDKR
VESKYGPP
(28) 12839B
Fab Light Chain
(SEQ ID NO: 382)
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
Fab Heavy Chain
(SEQ ID NO: 383)
QVQLVESGGGVVQPGRSLRLSCAASGFPFSNYVMYWVRQAPGKGLEWVALIFFDGKKNYHADSVKGRFTITRDNS
KNMLYLQMNSLRPEDAAVYYCAKIHCPNGVCYKGYYGMDVWGQGTTVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVDKKVE
PKSCDKTH;
(SEQ ID NO: 490)
QVQLVESGGGVVQPGRSLRLSCAASGFPFSNYVMYWVRQAPGKGLEWVALIFFDGKKNYHADSVKGRFTITRDNS
KNMLYLQMNSLRPEDAAVYYCAKIHCPNGVCYKGYYGMDVWGQGTTVTVSSASTKGPSVFPLAPCSRSTSESTAA
LGCLVKDYFPEPVTVSWNSGALTSGVHTFPAVLQSSGLYSLSSVVTVPSSSLGTKTYTCNVDHKPSNTKVDKRVE
SKYGPPLLQGSG
(SEQ ID NO: 499)
QVQLVESGGGVVQPGRSLRLSCAASGFPFSNYVMYWVRQAPGKGLEWVALIFFDGKKNYHADSVKGRFTITRDNS
KNMLYLQMNSLRPEDAAVYYCAKIHCPNGVCYKGYYGMDVWGQGTTVTVSSASTKGPSVFPLAPCSRSTSESTAA
LGCLVKDYFPEPVTVSWNSGALTSGVHTFPAVLQSSGLYSLSSVVTVPSSSLGTKTYTCNVDHKPSNTKVDKRVE
SKYGPP
(29) 12841B
Fab Light Chain
(SEQ ID NO: 384)
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
Fab Heavy Chain
(SEQ ID NO: 385)
EVQLVESGGGLVQPGGSLRLSCAASGFTFSNYWMNWVRQAPGKGLEWVANIKEDGGKKLYVDSVKGRFTISRDNA
KNSLFLQMNSLRAEDTAVYYCAREDTTLVVDYYYYGMDVWGQGTTVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEP
KSCDKTH
(30) 12850B
Fab Light Chain
(SEQ ID NO: 386)
EIVLTQSPGTLSLSPGERATLSCRASQSVSSSYLAWYQQKPGQAPRLLIYGASSRATGIPDRFSGSGSGTDFTLT
ISRLEPEDFAVYYCQQYGSSPWTFGQGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
Fab Heavy Chain
(SEQ ID NO: 387)
QVQLVQSGAEVKKPGSSVKVSCKASGGTFNTYAITWVRQAPGQGLEWMGGIIPISGIAEYAQKFQGRVTITTDDS
STTAYMELNSLRSEDTAVYYCASWNYALYYFYGMDVWGRGTTVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKSC
DKTH
(31) 69261
Fab Light Chain
(SEQ ID NO: 388)
DIVMTQSPLSLPVTPGEPASISCRSSQSLLHSNGYNYLDWYLQKPGQSPQFLIYLGSNRASGVPDRFSGSGSGTD
FTLKINRVEAEDVGVYYCMQALQTPYTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSENRGEC
Fab Heavy Chain
(SEQ ID NO: 389)
QVQLVESGGGLVKPGGSLRLSCAASGFTFSVYYMNWIRQAPGKGLEWVSYISSSGSTIYYADSVKGRFTISRDNA
KNSLYLQMNSLRAEDTAVYYCGREGYSGTYSYYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEPKS
CDKTH
(32) 69263
Fab Light Chain
(SEQ ID NO: 390)
DIQMTQSPSSLSASVGDRVTITCRASQDISHYSAWYQQKPGKLPNLLIYAASTLQSGVPSRESGSGSGTDESLTT
SSLQPEDVATYYCQKYNSVPLTFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
Fab Heavy Chain
(SEQ ID NO: 391)
EVQLVESGGGLVQPGRSLRLSCAVSGFTFDDYAMHWVRQAPGKGLEWVSGISWNSGTRGYADSVKGRFTISRDNA
KNSLYLQMNSLRGEDTALYYCVKDITISPNYYGMDVWGQGTTVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKSC
DKTH

In some embodiments, the anti-hTfR proteins described herein may comprise any of the exemplary hIgG1 heavy chain sequences provided in Table 1-3.

TABLE 1-3
hIgG1 Heavy Chain Sequences

| anti- | |
|---|---|
| hTfR: | |
| Molecule | HC Full hIgG1 sequence |
| 31874B | EVQLVESGGGLVQPGGSLRLSCAASGFAFSSYAMTWVRQAPGKGLEWVSVISGTGGSTY |
| | YADSVKGRFTISRDNSKNTLYLQMNSLRAEDTAVYYCAKGGAARRMEYFQYWGQGTLVT |
| | VSSASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTCPPCPAP |
| | ELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK(SEQ ID NO: 543) |
| 31863B | EVQLVESGGGLVQPGGSLRLSCAASGFTFNSYAMTWVRQAPGKGLEWVSFIGGSTGNTY |
| | YAGSVKGRFTISSDNSKKTLYLQMNSLRAEDTAVYYCAKGGAARRMEYFQHWGQGTLVT |
| | VSSASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTCPPCPAP |
| | ELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK(SEQ ID NO: 544) |
| 69348 | QVQLVESGGGVVQPGRSLRLSCAASGFTFTTYGMHWVRQAPGKGLEWVAVIWYDGSNKY |
| | YGDSVKGRFTISRDNSKNTLYLQMNSLRVDDTAVYYCTRTHGYTRSSDGFDYWGQGTLV |
| | TVSSASTKGPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK(SEQ ID NO: 545) |
| 69340 | EVQLVESGGGLVQPGRSLRLSCAASGFTFDDKAMHWVRQVPGKGLEWISGISWNSGTIG |
| | YADSVKGRFIISRDNAKNSLYLQMNSLRAEDTALYYCAKDGDTSGWYWYGLDVWGQGTT |
| | VTVSSASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK(SEQ ID NO: 546) |
| 69331 | QVQLVESGGGVVQPGRSLRLSCIASGFTFSVYGIHWVRQAPGKGLEWMAVISHDGNIKH |
| | YADSVKGRFTISRDNSKNTLYLQINSLRTEDTAVYYCAKDTWNSLDTFDIWGQGTMVTV |
| | SSASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKKVEPKSCDKTHTCPPCPAPE |
| | LLGGPSVFLEPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK(SEQ ID NO: 547) |
| 69332 | QVTLRESGPALVKPSQTLTLTCTFSGFSLNTYGMFVSWIRQPPGKALEWLAHIHWDDDK |
| | YYSTSLKTRLTISKDTSKNQVVLTMTNMDPVDTATYYCARGHNNLNYIIHWGQGTLVTV |
| | SSASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKKVEPKSCDKTHTCPPCPAPE |
| | LLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK(SEQ ID NO: 548) |
| 69326 | EVQLVESGGGLVQPGGSLRLSCAVSGFIFSSYEMNWVRQAPGKGLEWVSYISSSGSTIF |
| | YADSVKGRFTISRDNAKNSLYLQMNSLRAEDTAVYYCVSGVVLFDVWGQGTMVTVSSAS |
| | TKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTHTCPPCPAPELLGG |
| | PSVFLFPPKPKDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK(SEQ ID NO: 549) |
| 69329 | EVQLVESGGGLVQPGGSLRLSCAASGFTFSNYWMTWVRQAPGKGLEWVANIKEDGSEKD |
| | YVDSVKGRFTISRDNAKNSLYLQMNSLRGEDTAVYYCARDGEQLVDYYYYYVMDVWGQG |
| | TTVTVSSASTKGPSVFPLAPSSKSTSGGTAALGCLVKDYFPEPVTVSWNSGALTSGVHT |
| | FPAVLQSSGLYSLSSVVTVPSSSLGTQTYICNVNHKPSNTKVDKKVEPKSCDKTHTCPP |
| | CPAPELLGGPSVFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK(SEQ ID NO: 550) |
| 69323 | EVQLVESGGGLVQPGRSLRLSCAASGFTFDDYAMHWVRQAPGKGLEWVSGISWNSGYIG |
| | YADSVKGRFTISRDNAENSLHLQMNSLRAEDTALYYCARGGSTLVRGVKGGYYGMDVWG |
| | QGTTVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKKVEPKSCDKTHTC |
| | PPCPAPELLGGPSVFLFPPKPKDTLMISRTPEVTCVVVDVSHEDPEVKFNWYVDGVEVH |
| | NAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK(SEQ ID NO: |
| | 551) |
| 69305 | QVQLVESGGGVVQPGRSLRLSCAASGFTFSSYGMHWVRQAPGKGLEWVAVIWYDGSNKY |
| | YADSVKGRFTISRDISKNTLYLQMNSLRAEDTAVYYCAGQLDLFFDYWGQGTLVTVSSA |
| | STKGPSVFPLAPSSKSTSGGTAALGCLVKDYFPEPVTVSWNSGALTSGVHTFPAVLQSS |
| | GLYSLSSVVTVPSSSLGTQTYICNVNHKPSNTKVDKKVEPKSCDKTHTCPPCPAPELLG |
| | GPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK(SEQ ID NO: 552) |
| 69307 | EVQLVESGGGLVQPGGSLRLSCTASGFTFSNYWMTWVRQAPGKGLEWVANIKEDGSEKE |
| | YVDSVKGRFTISRDNAKNSLYLQMNSLRGEDTAVYYCARDGEQLVDYYYYYVMDVWGQG |
| | TTVTVSSASTKGPSVFPLAPSSKSTSGGTAALGCLVKDYFPEPVTVSWNSGALTSGVHT |
| | FPAVLQSSGLYSLSSVVTVPSSSLGTQTYICNVNHKPSNTKVDKKVEPKSCDKTHTCPP |
| | CPAPELLGGPSVFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK(SEQ ID NO: 553) |
| 12795B | EVQLVESGGGLVQPGGSLRLSCATSGFTFTSYDMKWVRQAPGLGLEWVSAISGSGGNTY |
| | YADSVKGRFTISRDNSRNTLYLQMNSLRAEDTAVYYCTRSHDFGAFDYFDYWGQGTLVT |
| | VSSASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTCPPCPAP |
| | ELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK(SEQ ID NO: 554) |
| 12798B | EVQLVESGGDLVQPGRSLRLSCAASGFTFDDYAMHWVRQAPGKGLEWVSGISWNSATRV |
| | YADSVKGRFTISRDNAKNFLYLQMNSLRSEDTALYHCAKDMDISLGYYGLDVWGQGTTV |
| | TVSSASTKGPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK(SEQ ID NO: 555) |
| 12799B | QITLKESGPTLVKPTQTLTLTCTFSGFSLSTSGVGVVWIRQPPGKALEWLALIYWNDHK |
| | RYSPSLGSRLTITKDTSKNQVVLTMTNMDPVDTATYYCAHYSGSYSYYYYGLDVWGQGT |
| | TVTVSSASTKGPSVFPLAPSSKSTSGGTAALGCLVKDYFPEPVTVSWNSGALTSGVHTF |
| | PAVLQSSGLYSLSSVVTVPSSSLGTQTYICNVNHKPSNTKVDKKVEPKSCDKTHTCPPC |
| | PAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK(SEQ ID NO: 556) |
| 12801B | EVQLLESGGALVQPGGSLRLSCAASGFTFTSYAMHWVRQAPGKGLEWVSSIRGSGGGTY |
| | SADSVKGRFTISRDNSRDTLYLQMNSVRAEDTAVYYCARSHDYGAFDFFDYWGQGTLVT |
| | VSSASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTCPPCPAP |
| | ELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK(SEQ ID NO: 557) |
| 12802B | QVQLVESGGGLVKPGGSLRLSCAASGFTFSDYFMSWIRQAPGKGLEWVSYISSTGSTIN |
| | YADSVKGRFTISRDNVKNSLYLQMTSLRVEDTAVYYCTRDNWNYEYWGQGTLVTVSSAS |
| | TKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTHTCPPCPAPELLGG |
| | PSVFLFPPKPKDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK(SEQ ID NO: 558) |
| 12808B | QLQLQESGPGLVKPSETLSLTCTVSGESISSNTYYWGWIRQPPGKGLEWIGSIDYSGTT |
| | NYNPSLKSRVTISVDTSRNHFSLRLRSVTAADTAVYYCAREWGNYGYYYGMDVWGQGTT |
| | VTVSSASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK(SEQ ID NO: 559) |
| 12812B | QVQLVQSGAEVKKPGSSVRVSCKASRGTFSSYAISWVRQAPGQGLEWMGGIIPIFGTAN |
| | YAQKFLARVTITADESTSTAYMELSSLRSEDTAVYYCAREKGWNYFDYWGQGTLVTVSS |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK(SEQ ID NO: 560) |
| 12816B | QVQLVESGGGLVKPGGSLRLSCAASGFTFSDYYMNWIRQAPGKGLEWVSYISSSGTTIY |
| | YADSVKGRFTISRDNAKKSLYLEMNSLRAEDTAVYYCAREGYGNDYYYYGIDVWGQGTT |
| | VTVSSASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDKTHTCPPCP |
| | APELLGGPSVFLFPPKPKDTLMISRTPEVTCVWVDVSHEDPEVKFNWYVDGVEVHNAKT |
| | KPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK(SEQ ID NO: 561) |
| 12833B | QVQLVESGGGVVQPGRSLRLSCAASGFTFSSFGMHWVRQAPGKGLEWVIFISYDGSDKY |
| | YADSVKGRFAISRDSSKNTLYLQMNSLRAEDTAVYYCAKENGILTDSYGMDVWGQGTTV |
| | TVSSASTKGPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK(SEQ ID NO: 562) |
| 12834B | QVQLVQSGAEVKKPGASVKVSCKASGYTFTSYGISWVRQAPGQGLEWMGWISVYHGNTN |
| | YAQKFQGRVTMTTDTSTSTAYMELRSLRSDDTAVYYCAREGYYDFWSGYYPFDYWGQGT |
| | LVTVSSASTKGPSVFPLAPSSKSTSGGTAALGCLVKDYFPEPVTVSWNSGALTSGVHTF |
| | PAVLQSSGLYSLSSVVTVPSSSLGTQTYICNVNHKPSNTKVDKKVEPKSCDKTHTCPPC |
| | PAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK(SEQ ID NO: 563) |
| 12835B | EVQLVESGGGLIQPGGSLRLSCEASGFTFRNYEMNWVRQAPGKGLEWVSYISSSGNMKD |
| | YAESVKGRFTISRDNVKNSLQLQMNSLRVEDTAVYYCARDEFPYGMDVWGQGTTVTVSS |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK(SEQ ID NO: 564) |
| 12847B | EVQLVESGGGLVQPGRSLRLSCAASGFTFDDYAMNWVRQAPGKGLEWVSGISWSSGSMD |
| | YADSVKGRFTISRDNAKNSLYLQMNSLRTEDTALYYCAKAREVGDYYGMDVWGQGTTVT |
| | VSSASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTCPPCPAP |
| | ELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK(SEQ ID NO: 565) |
| 12848B | EVQLVESGGGLVQPGRSLTLSCAASGFTFDNFGMHWVRQGPGKGLEWVSGLTWNSGVIG |
| | YADSVKGRFTISRDNAKNSLYLQMNSLRPEDTALYYCAKDIRNYGPFDYWGQGTLVTVS |
| | SASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKKVEPKSCDKTHTCPPCPAPEL |
| | LGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLT |
| | VDKSRWQQGNVFSCSVMHEALHNHYTQKSLSLSPGK(SEQ ID NO: 566) |
| 12843B | EVQLVESGGGLVQPGGSLRLSCAASGFTFNIFEMNWVRQAPGKGLEWISYISSRGTTTY |
| | YADSVRGRFTISRDNAKNSLYLQMNSLRAEDTAVYYCARDYEATIPFDFWGQGTLVTVS |
| | SASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKKVEPKSCDKTHTCPPCPAPEL |
| | LGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLT |
| | VDKSRWQQGNVFSCSVMHEALHNHYTQKSLSLSPGK(SEQ ID NO: 567) |
| 12844B | EVQLVESGGSVVRPGGSLRLSCEASGFTFDDYGMSWVRQDPGKGLEWVSGINWNGDRTN |
| | YADSVKGRFIISRDNAKNSVYLQMNSLRAEDSALYHCARDQGLGVAATLDYWGQGTLVT |
| | VSSASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTCPPCPAP |
| | ELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK(SEQ ID NO: 568) |
| 12845B | EVQLVESGGGLVQPGGSLRLSCAASGFTVSNYEMNWVRQAPGKGLEWVSYISSSTSNIY |
| | YADSVKGRFTISRDNAENSLYLQMNSLRVEDTAVYYCVRDGIVWVPVGRGYYYYGLDVW |
| | GQGTTVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKKVEPKSCDKTHT |
| | CPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK(SEQ ID NO: |
| | 569) |
| 12839B | QVQLVESGGGVVQPGRSLRLSCAASGFPFSNYVMYWVRQAPGKGLEWVALIFFDGKKNY |
| | HADSVKGRFTITRDNSKNMLYLQMNSLRPEDAAVYYCAKIHCPNGVCYKGYYGMDVWGQ |
| | GTTVTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKSCDKTHTCP |
| | PCPAPELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPRE |
| | PQVYTLPPSRDELTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK(SEQ ID NO: 570) |
| 12841B | EVQLVESGGGLVQPGGSLRLSCAASGFTFSNYWMNWVRQAPGKGLEWVANIKEDGGKKL |
| | YVDSVKGRFTISRDNAKNSLFLQMNSLRAEDTAVYYCAREDTTLVVDYYYYGMDVWGQG |
| | TTVTVSSASTKGPSVFPLAPSSKSTSGGTAALGCLVKDYFPEPVTVSWNSGALTSGVHT |
| | FPAVLQSSGLYSLSSVVTVPSSSLGTQTYICNVNHKPSNTKVDKKVEPKSCDKTHTCPP |
| | CPAPELLGGPSVFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK(SEQ ID NO: 571) |
| 12850B | QVQLVQSGAEVKKPGSSVKVSCKASGGTFNTYAITWVRQAPGQGLEWMGGIIPISGIAE |
| | YAQKFQGRVTITTDDSSTTAYMELNSLRSEDTAVYYCASWNYALYYFYGMDVWGRGTTV |
| | TVSSASTKGPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK(SEQ ID NO: 572) |
| 69261 | QVQLVESGGGLVKPGGSLRLSCAASGFTFSVYYMNWIRQAPGKGLEWVSYISSSGSTIY |
| | YADSVKGRFTISRDNAKNSLYLQMNSLRAEDTAVYYCGREGYSGTYSYYGMDVWGQGTT |
| | VTVSSASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK(SEQ ID NO: 573) |
| 69263 | EVQLVESGGGLVQPGRSLRLSCAVSGFTFDDYAMHWVRQAPGKGLEWVSGISWNSGTRG |
| | YADSVKGRFTISRDNAKNSLYLQMNSLRGEDTALYYCVKDITISPNYYGMDVWGQGTTV |
| | TVSSASTKGPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK(SEQ ID NO: 574) |

In some embodiments, the anti-TfR proteins described herein may comprise a IgG1 heavy chain constant domain comprising the amino acid sequence of ASTKGPSVFPLAPSSKSTSGGTAALGCLVKDYFPEPVTVSWNSGALTSGVHTFPAVLQ SSGLYSLSSVVTVPSSSLGTQTYICNVNHKPSNTKVDKKVEPKSCDKTHTCPPCPAPEL LGGPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTL PPSRDELTKNQVSLTCLVKGFYPSDIAVEWESNGQPENNYKTTPPVLDSDGSFFLYSK LTVDKSRWQQGNVFSCSVMHEALHNHYTQKSLSLSPGK (SEQ ID NO: 575), or a variant thereof.

“31874B”; “31863B”; “69348”; “69340”; “69331”; “69332”; “69326”; “69329”; “69323”; “69305”; “69307”; “12795B”; “12798B”; “12799B”; “12801B”; “12802B”; “12808B”; “12812B”; “12816B”; “12833B”; “12834B”; “12835B”; “12847B”; “12848B”; “12843B”; “12844B”; “12845B”; “12839B”; “12841B”; “12850B”; “69261”; and “69263” refer to anti-TfR protein-drug conjugates, e.g., anti-TfR scFv or anti-TfR Fab, comprising a light chain variable region comprising the amino acid sequence set forth in SEQ ID NO: 7; 17; 27; 37; 465; 47; 466; 57; 468; 67; 469; 77; 471; 87; 97; 107; 117; 474; 127; 137; 147; 476; 157; 167; 177; 187; 479; 197; 207; 217; 227; 237; 247; 257; 267; 277; 287; 297; 307; 527; 317 or 484 (or a variant thereof), and a heavy chain variable region comprising the amino acid sequence set forth in SEQ ID NO: 2; 462; 12; 463; 22; 464; 32; 42; 52; 467; 62; 492; 72; 470; 82; 92; 472; 102; 112; 473; 122; 132; 142; 475; 152; 162; 477; 172; 182; 478; 192; 480; 202; 481; 212; 222; 232; 242; 252; 482; 262; 272; 282; 292; 302; 483 or 312 (or a variant thereof), which, in the case of an scFv, can be fused together (in either order), e.g., by a peptide linker (e.g., (G₄S)₃ (SEQ ID NO: 541)), respectively; or that comprise a V_{H} that comprises the CDRs thereof (CDR-H1 (or a variant thereof), CDR-H2 (or a variant thereof) and CDR-H3 (or a variant thereof)) and/or a V_{L} that comprises the CDRs thereof (CDR-L1 (or a variant thereof), CDR-L2 (or a variant thereof) and CDR-L3 (or a variant thereof)), wherein the V_{H} fused to the V_{L} or the V_{L} fused to the V_{H}, in the case of an scFv, can be conjugated, e.g., by a linker, to a molecular cargo.

The term “fused” or “tethered” with regard to fused polypeptides refers to polypeptides joined directly or indirectly (e.g., via a linker or other polypeptide).

In some embodiments, the anti-TfR antigen-binding protein described herein comprises a humanized antibody or antigen binding fragment thereof, murine antibody or antigen binding fragment thereof, chimeric antibody or antigen binding fragment thereof, monoclonal antibody or antigen binding fragment thereof (e.g., monovalent Fab′, divalent Fab2, F(ab)′3 fragments, single-chain variable fragment (scFv), bis-scFv, (scFv)2, diabody, bivalent antibody, one-armed antibody, minibody, nanobody, triabody, tetrabody, disulfide stabilized Fv protein (dsFv), single-domain antibody (sdAb), Ig NAR, camelid antibody or antigen binding fragment thereof, bispecific antibody or biding fragment thereof, (e.g., bisscFv, or a bi-specific T-cell engager (BiTE)), trispecific antibody (e.g., F(ab)′3 fragments or a triabody), or a chemically modified derivative thereof.

The term “humanized antibody”, as used herein, includes antibodies in which CDR sequences derived from the germline of another mammalian species, such as a mouse, have been grafted onto human framework sequences, or otherwise modified to increase their similarity to antibody variants produced naturally in humans.

In some cases, the anti-TfR antigen-binding protein is an antibody which comprises one or more mutations in a framework region, e.g., in the CH1 domain, CH2 domain, CH3 domain, hinge region, or a combination thereof. In some embodiments, the one or more mutations are to stabilize the antibody and/or to increase half-life. In some embodiments, the one or more mutations are to modulate Fc receptor interactions, to reduce or eliminate Fc effector functions such as FcγR, antibody-dependent cell-mediated cytotoxicity (ADCC), or complement-dependent cytotoxicity (CDC). In additional embodiments, the one or more mutations are to modulate glycosylation.

In some embodiments, one, two or more mutations (e.g., amino acid substitutions) are introduced into the Fc region of an antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity. In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., PCT Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo. In some embodiments, the Fc region comprises a mutation at residue position L234, L235, or a combination thereof. In some embodiments, the mutations comprise L234 and L235. In some embodiments, the mutations comprise L234A and L235A.

The anti-TfR antibodies and antigen-binding fragments described herein may be modified after translation, e.g., glycosylated.

For example, antibodies and antigen-binding fragments described herein may be glycosylated (e.g., N-glycosylated and/or O-glycosylated) or aglycosylated. Typically, antibodies and antigen-binding fragments are glycosylated at the conserved residue N297 of the IgG Fc domain. Some antibodies and fragments include one or more additional glycosylation sites in a variable region. In an embodiment, the glycosylation site is in the following context: FN₂₉₇S or YN₂₉₇S.

In an embodiment, said glycosylation is any one or more of three different N-glycan types: high mannose, complex and/or hybrid that are found on IgGs with their respective linkage. Complex and hybrid types exist with core fucosylation, addition of a fucose residue to the innermost N-acetylglucosamine, and without core fucosylation.

In some cases, the anti-TfR antigen-binding protein is an aglycosylated antibody, i.e., an antibody that does not comprise a glycosylation sequence that might interfere with a transglutamination reaction, for instance an antibody that does not have a saccharide group at N297 on one or more heavy chains according to the EU numbering system (or position N180 with reference to the amino acid sequence of SEQ ID NO: 575). In particular embodiments, an antibody heavy chain has an N297 mutation (or position N180 with reference to the amino acid sequence of SEQ ID NO: 575). In particular embodiments, an antibody heavy chain has an N297Q or an N297D mutation (or N180Q or an N180D mutation with reference to the amino acid sequence of SEQ ID NO: 575). The N-linked glycan found at position 297 can be found as a core structure, common to all IgG found in human beings and rodents. Antibodies comprising such above-described mutations can be prepared by site-directed mutagenesis to remove or disable a glycosylation sequence or by site-directed mutagenesis to insert a glutamine residue at site apart from any interfering glycosylation site or any other interfering structure. Such antibodies also can be isolated from natural or artificial sources. Aglycosylated antibodies also include antibodies comprising a T299 or S298P or other mutations, or combinations of mutations that result in a lack of glycosylation.

In some cases, the antigen-binding protein is a deglycosylated antibody, i.e., an antibody in which a saccharide group at is removed to facilitate transglutaminase-mediated conjugation. Saccharides include, but are not limited to, N-linked oligosaccharides. In some embodiments, deglycosylation is performed at residue N180 (with reference to the amino acid sequence of SEQ ID NO: 575). In some embodiments, deglycosylation is performed at residue N297 chains according to the EU numbering system. In some embodiments, removal of saccharide groups is accomplished enzymatically, included but not limited to via PNGase.

In an embodiment, an antibody or fragment described herein is afucosylated.

The antibodies and antigen-binding fragments described herein may also be post-translationally modified in other ways including, for example: Glu or Gln cyclization at N-terminus; Loss of positive N-terminal charge; Lys variants at C-terminus; Deamidation (Asn to Asp); Isomerization (Asp to isoAsp); Deamidation (GIn to Glu); Oxidation (Cys, His, Met, Tyr, Trp); and/or Disulfide bond heterogeneity (Shuffling, thioether and trisulfide formation).

In some embodiments, an antibody disclosed herein comprises Q295 which can be native to the antibody heavy chain sequence. In some embodiments, an antibody heavy chain disclosed herein may comprise Q295. In some embodiments, an antibody heavy chain disclosed herein may comprise Q295 and an amino acid substitution N297D.

According to certain embodiments of the present disclosure, anti-TfR antibodies and antigen-binding fragments are provided comprising an Fc domain comprising one or more mutations which enhance or diminish antibody binding to the FcRn receptor, e.g., at acidic pH as compared to neutral pH. For example, the present disclosure includes anti-TfR antibodies comprising a mutation in the CH2 or a CH3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal.

Non-limiting examples of such Fc modifications include, e.g., a modification at position:
250 (e.g., E or Q);
250 and 428 (e.g., L or F);
252 (e.g., L/Y/F/W or T),
254 (e.g., S or T), and/or
256 (e.g., S/R/Q/E/D or T);
and/or a modification at position:
428 and/or 433 (e.g., H/L/R/S/P/Q or K), and/or
434 (e.g., A, W, H, F or Y);
and/or a modification at position:
250 and/or 428;
and/or a modification at position:
307 or 308 (e.g., 308F, V308F), and/or 434.

In an embodiment, the modification comprises:
a 428L (e.g., M428L) and 434S (e.g., N434S) modification;
a 428L, 2591 (e.g., V259I), and 308F (e.g., V308F) modification;
a 433K (e.g., H433K) and a 434 (e.g., 434Y) modification;
a 252, 254, and 256 (e.g., 252Y, 254T, and 256E) modification;
a 250Q and 428L modification (e.g., T250Q and M428L); and/or
a 307 and/or 308 modification (e.g., 308F or 308P).

For example, the present disclosure includes anti-TfR antibodies comprising an Fc domain comprising one or more pairs or groups of mutations selected from the group consisting of:
250Q and 248L (e.g., T250Q and M248L);
252Y, 254T and 256E (e.g., M252Y, S254T and T256E);
2571 and 3111 (e.g., P2571 and Q3111);
2571 and 434H (e.g., P2571 and N434H);
376V and 434H (e.g., D376V and N434H);
307A, 380A and 434A (e.g., T307A, E380A and N434A);
428L and 434S (e.g., M428L and N434S); and
433K and 434F (e.g., H433K and N434F).

In yet another embodiment, the modification comprises a 265A (e.g., D265A) and/or a 297A (e.g., N297A) modification.

In an embodiment, the heavy chain constant domain is gamma4 comprising an S228P and/or S108P mutation. See Angal et al., A single amino acid substitution abolishes the heterogeneity of chimeric mouse/human (IgG4) antibody, Mol Immunol. 1993 January; 30(1):105-108.

All possible combinations of the foregoing Fc domain mutations, and other mutations within the antibody variable domains disclosed herein, are contemplated within the scope of the present disclosure.

The anti-TfR antibodies described herein may comprise a modified Fc domain having reduced effector function. As used herein, a “modified Fc domain having reduced effector function” means any Fc portion of an immunoglobulin that has been modified, mutated, truncated, etc., relative to a wild-type, naturally occurring Fc domain such that a molecule comprising the modified Fc exhibits a reduction in the severity or extent of at least one effect selected from the group consisting of cell killing (e.g., ADCC and/or CDC), complement activation, phagocytosis and opsonization, relative to a comparator molecule comprising the wild-type, naturally occurring version of the Fc portion. In certain embodiments, a “modified Fc domain having reduced effector function” is an Fc domain with reduced or attenuated binding to an Fc receptor (e.g., FcγR).

In certain embodiments, the modified Fc domain is a variant IgG1 Fc or a variant IgG4 Fc comprising a substitution in the hinge region. For example, a modified Fc for use in the context of the present disclosure may comprise a variant IgG1 Fc wherein at least one amino acid of the IgG1 Fc hinge region is replaced with the corresponding amino acid from the IgG2 Fc hinge region. Alternatively, a modified Fc for use in the context of the present disclosure may comprise a variant IgG4 Fc wherein at least one amino acid of the IgG4 Fc hinge region is replaced with the corresponding amino acid from the IgG2 Fc hinge region. Non-limiting, exemplary modified Fc regions that can be used in the context of the present disclosure are set forth in US Patent Application Publication No. 2014/0243504, the disclosure of which is hereby incorporated by reference in its entirety, as well as any functionally equivalent variants of the modified Fc regions set forth therein.

The present disclosure also includes antigen-binding proteins, antibodies or antigen-binding fragments, comprising a HCVR set forth herein and a chimeric heavy chain constant (CH) region, wherein the chimeric CH region comprises segments derived from the CH regions of more than one immunoglobulin isotype. For example, the antibodies of the disclosure may comprise a chimeric CH region comprising part or all of a CH2 domain derived from a human IgG1, human IgG2 or human IgG4 molecule, combined with part or all of a CH3 domain derived from a human IgG1, human IgG2 or human IgG4 molecule. According to certain embodiments, the antibodies of the disclosure comprise a chimeric CH region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric CH region as described herein may, in certain embodiments, exhibit modified Fc effector functions without adversely affecting the therapeutic or pharmacokinetic properties of the antibody. (See, e.g., WO2014/022540).

Other modified Fc domains and Fc modifications that can be used in the context of the present disclosure include any of the modifications as set forth in US2014/0171623; U.S. Pat. No. 8,697,396; US2014/0134162; WO2014/043361, the disclosures of which are hereby incorporated by reference in their entireties. Methods of constructing antibodies or other antigen-binding fusion proteins comprising a modified Fc domain as described herein are known in the art.

In some embodiments, the anti-TfR antibodies and antigen-binding fragments described herein comprise an Fc domain comprising one or more mutations in the CH2 and/or CH3 regions that generate a separate TfR binding site.

In an embodiment, the CH2 region comprises one or more amino acid mutations, or a combination thereof, selected from the following: a) position 47 is Glu, Gly, Gln, Ser, Ala, Asn, Tyr, or Trp; position 49 is Ile, Val, Asp, Glu, Thr, Ala, or Tyr; position 56 is Asp, Pro, Met, Leu, Ala, Asn, or Phe; position 58 is Arg, Ser, Ala, or Gly; position 59 is Tyr, Trp, Arg, or Val; position 60 is Glu; position 61 is Trp or Tyr; position 62 is Gln, Tyr, His, Ile, Phe, Val, or Asp; and position 63 is Leu, Trp, Arg, Asn, Tyr, or Val; b) position 39 is Pro, Phe, Ala, Met, or Asp; position 40 is Gln, Pro, Arg, Lys, Ala, Ile, Leu, Glu, Asp, or Tyr; position 41 is Thr, Ser, Gly, Met, Val, Phe, Trp, or Leu; position 42 is Pro, Val, Ala, Thr, or Asp; position 43 is Pro, Val, or Phe; position 44 is Trp, Gln, Thr, or Glu; position 68 is Glu, Val, Thr, Leu, or Trp; position 70 is Tyr, His, Val, or Asp; position 71 is Thr, His, Gln, Arg, Asn, or Val; and position 72 is Tyr, Asn, Asp, Ser, or Pro; c) position 41 is Val or Asp; position 42 is Pro, Met, or Asp; position 43 is Pro or Trp; position 44 is Arg, Trp, Glu, or Thr; position 45 is Met, Tyr, or Trp; position 65 is Leu or Trp; position 66 is Thr, Val, Ile, or Lys; position 67 is Ser, Lys, Ala, or Leu; position 69 is His, Leu, or Pro; and position 73 is Val or Trp; or d) position 45 is Trp, Val, Ile, or Ala; position 47 is Trp or Gly; position 49 is Tyr, Arg, or Glu; position 95 is Ser, Arg, or Gln; position 97 is Val, Ser, or Phe; position 99 is Ile, Ser, or Trp; position 102 is Trp, Thr, Ser, Arg, or Asp; position 103 is Trp; and position 104 is Ser, Lys, Arg, or Val; wherein the substitutions and the positions are determined with reference to amino acids 4-113 of

(SEQ ID NO: 540)
PCPAPELLGGPSVELFPPKPKDTLMISRTPEVTCVVVDVSHEDPEVKENW
YVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSV
MHEALHNHYTQKSLSLSPGK.

In an embodiment, the CH3 region comprises one or more amino acid mutations, or a combination thereof, selected from the following: position 153 is Trp, Leu, or Glu; position 157 is Tyr or Phe; position 159 is Thr; position 160 is Glu; position 161 is Trp; position 162 is Ser, Ala, Val, or Asn; position 163 is Ser or Asn; position 186 is Thr or Ser; position 188 is Glu or Ser; position 189 is Glu; and position 194 is Phe; or b) position 118 is Phe or IIe; position 119 is Asp, Glu, Gly, Ala, or Lys; position 120 is Tyr, Met, Leu, Ile, or Asp; position 122 is Thr or Ala; position 210 is Gly; position 211 is Phe; position 212 is His, Tyr, Ser, or Phe; and position 213 is Asp; wherein the substitutions and the positions are determined with reference to amino acids 114-220 of SEQ ID NO: 540.

In some embodiments, the CH3 region comprises one or more mutations, or a combination thereof, selected from the following: position 384 is Leu, Tyr, Met, or Val; position 386 is Leu, Thr, His, or Pro; position 387 is Val, Pro, or an acidic amino acid; position 388 is Trp; position 389 is Val, Ser, or Ala; position 413 is Glu, Ala, Ser, Leu, Thr, or Pro; position 416 is Thr or an acidic amino acid; and position 421 is Trp, Tyr, His, or Phe, according to EU numbering. In an embodiment, the CH3 region comprises one or more amino acid mutations, or a combination thereof, selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe, according to EU numbering.

In some embodiments, the CH3 region comprises one or more mutations, or a combination thereof, selected from the following: a) Phe at position 382, Tyr at position 383, Asp at position 384, Asp at position 385, Ser at position 386, Lys at position 387, Leu at position 388, Thr at position 389, Pro at position 419, Arg at position 420, Gly at position 421, Leu at position 422, Ala at position 424, Glu at position 426, Tyr at position 438, Leu at position 440, Gly at position 442, and Glu at position 443; b) Phe at position 382, Tyr at position 383, Gly at position 384, N at position 385, Ala at position 386, Lys at position 387, Thr at position 389, Leu at position 422, Ala at position 424, Glu at position 426, Tyr at position 438, Leu at position 440; c) Phe at position 382, Tyr at position 383, Glu at position 384, Ala at position 385, Lys at position 387, Leu at position 388, Leu at position 422, Ala at position 424, Glu at position 426, Tyr at position 438, Leu at position 440; d) Phe at position 382, Glu at position 384, Ser at position 386, Lys at position 387, Thr at position 389, Leu at position 422, Ala at position 424, Glu at position 426, Tyr at position 438, Leu at position 440; e) Phe at position 382, Gly at position 384, Ala at position 385, Lys at position 387, Ser at position 389, Leu at position 422, Ala at position 424, Glu at position 426, Tyr at position 438, Leu at position 440; f) Phe at position 382, Gly at position 384, Ala at position 385, Lys at position 387, Leu at position 388, Thr at position 389, Leu at position 422, Ala at position 424, Glu at position 426, Tyr at position 438, Leu at position 440; wherein the positions are determined according to EU numbering.

Additional mutations in CH2 and/or CH3 regions that can introduce non-native TfR binding sites into the antigen-binding proteins descried herein include those described in US Patent Application Publication Nos. 2020/0223935, 2020/0369746, 2021/0130485, 2022/0017634; and PCT Application Publications Nos. WO2023/279099, WO2023/114499 and WO2023/114510, which are incorporated herein by reference in their entireties.

Also provided herein is a vessel (e.g., a plastic or glass vial, e.g., with a cap or a chromatography column, hollow bore needle or a syringe cylinder) comprising an anti-TfR protein-drug conjugates, e.g., anti-TfR scFv-drug conjugates or anti-TfR Fab-drug conjugates, described herein, e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841 B; 12850B; 69261; and 69263.

Also provided herein is an injection device comprising an anti-TfR protein-drug conjugate, e.g., anti-TfR scFv drug conjugates or anti-TfR Fab drug conjugates described herein, e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, or a pharmaceutical composition thereof. The injection device may be packaged into a kit. An injection device is a device that introduces a substance into the body of a subject via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., comprising the protein-drug conjugate or a pharmaceutical composition thereof), a needle for piercing skin and/or blood vessels or other tissue for injection of the fluid; and a plunger for pushing the fluid out of the cylinder and through the needle bore and into the body of the subject.

Further provided herein are methods for administering an anti-TfR protein-drug conjugate, e.g., anti-TfR scFv-drug conjugates or anti-TfR Fab-drug conjugates described herein, e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841 B; 12850B; 69261; or 69263, to a subject, comprising introducing the protein-drug conjugate into the body of the subject (e.g., a human), for example, parenterally (e.g., intravenously). For example, the method comprises piercing the body of the subject with a needle of a syringe and injecting the antigen-binding protein into the body of the subject, e.g., into the vein, artery, tumor, muscular tissue or subcutis of the subject.

Further provided herein are methods for delivering a molecular cargo wherein the molecular cargo is conjugated to, e.g., an antigen-binding protein described herein, e.g., anti-TfR scFv anti-TfR Fab described herein, e.g., 31874B; 31863B; 69348; 69340; 69331; 69332; 69326; 69329; 69323; 69305; 69307; 12795B; 12798B; 12799B; 12801 B; 12802B; 12808B; 12812B; 12816B; 12833B; 12834B; 12835B; 12847B; 12848B; 12843B; 12844B; 12845B; 12839B; 12841B; 12850B; 69261; or 69263, to a targeted tissue in a subject (e.g., any of the tissues or cell types or cell types in or associated with the corresponding tissues as set forth in Table 1-4 below; or brain, cerebral cortex; cerebellum; hippocampus; caudate; parathyroid gland; adrenal gland; bronchus; lung; oral mucosa; esophagus; stomach; duodenum; small intestine; colon; rectum; liver; gallbladder; pancreas; kidney; urinary bladder; testis; epididymis; prostate; vagina; ovary; fallopian tube; endometrium; cervix; placenta; breast; muscle, (e.g., heart muscle; skeletal muscle, smooth muscle and/or endothelial vasculature thereof); soft tissue; skin; appendix; lymph node; tonsil; and/or bone marrow), comprising introducing the protein-drug conjugate into the body of the subject (e.g., a human), for example, parenterally (e.g., intravenously). For example, the method comprises piercing the body of the subject with a needle of a syringe and injecting the protein-drug conjugate into the body of the subject, e.g., into the vein, artery, tumor, muscular tissue or subcutis of the subject. For example, the protein-drug conjugate may be introduced into the subject via intrathecal, intracerebroventricular, or intraparenchymal injection into the central nervous system.

TABLE 1-4
Tissue And Cell Types Which Can Be Targeted
For Delivery Of A Molecular Cargo Using A
Described Anti-TfR Antigen-Binding Protein.

| Target tissue | Cell types |
|---|---|
| Brain/Spinal cord/CNS | endothelial cells |
| | neurons (all types) |
| | oligodendrocytes (and precursors) |
| | pericytes |
| | meninges/leptomeningeal cells |
| | arachnoid barrier cells |
| | peripheral glia |
| | astrocytes |
| | glia |
| | Schwann cells |
| | ependymal cells |
| | microglia |
| Eye | rod photoreceptor cells |
| | muller glia cells |
| | bipolar cells |
| | cone photoreceptor cells |
| | endothelial cells |
| | cornea |
| | sclera |
| | optic nerve |
| | pupillary sphincter |
| Skeletal Muscle | skeletal myocytes |
| | fibroblasts |
| | endothelial cells |
| | macrophages |
| | satellite cells |
| Adipose tissue | Adipocytes |
| | fibroblasts |
| | T-cells |
| | Macrophages |
| | B-cells |
| | Dendritic cells |
| Blood/Bone marrow | T-cells |
| | B-cells |
| | Macrophages |
| | erythroid cells |
| | plasmid cells |
| | dendritic cells |
| Breast | glandular cells |
| | T-cells |
| | fibroblasts |
| | macrophages |
| | endothelial cells |
| | myoepithelial cells |
| | Adipocytes |
| Lung/Bronchus | Basal respiratory cells |
| | respiratory cilliated cells |
| | club cells |
| | smooth muscle cells |
| | ionocytes |
| | macrophages |
| | alveolar cells (type 1 & 2) |
| | T-cells |
| | endothelial cells |
| Colon | distal enterocytes |
| | intestinal goblet cells |
| | undifferentiated cells |
| | T-cells |
| | Paneth cells |
| | B-cells |
| | enteroednocrine cells |
| Uterus | glandular and luminal cells |
| | endometrial stromal cells |
| | endothelial cells |
| | smooth muscle cells |
| | T-cells |
| | macrophages |
| Esophagus | fibroblasts |
| | squamous epithelial cells |
| | endothelial cells |
| | smooth muscle cells |
| | macrophages |
| | plasma cells |
| | T-cells |
| Heart | cardiomyocytes |
| | endothelial cells |
| | fibroblasts |
| | macrophages |
| | T-cells |
| | B-cells |
| | Dendritic cells |
| Kidney | proximal tubular cells |
| | T-cells |
| | macrophages |
| | collecting duct cells |
| | B-cells |
| | glomeruli |
| | fibroblasts |
| Liver | hepatocytes |
| | B-cells |
| | erythroid cells |
| Lymph node | B-cells |
| | T-cells |
| Ovary | granulosa cells |
| | fibroblasts |
| | smooth muscle cells |
| | macrophages |
| | T-cells |
| | theca cells |
| | fibroblasts |
| Pancreas | ductal cells |
| | pancreatic endocrine cells |
| | smooth muscle cells |
| | endothelial cells |
| | macrophages |
| | exocrine glandular cells |
| | monocytes |
| Placenta | cytotrophoblasts |
| | extravillous trophoblasts |
| | fibroblasts |
| | hofbauer cells |
| | endothelial cells |
| Prostate | basal prostatic cells |
| | prostatic glandular cells |
| | urothelial cells |
| | endothelial cells |
| | fibroblasts |
| | smooth muscle cells |
| | macrophages |
| | T-cells |
| Rectum | undifferentiated cells |
| | intestinal goblet cells |
| | Paneth cells |
| | distal enterocytes |
| | enteroednocrine cells |
| Skin | langerhans cells |
| | fibroblasts |
| | endothelial cells |
| | basal keratinocytes |
| | suprabasal keratinocytes |
| | T-cells |
| | smooth muscle cells |
| | melanocytes |
| PBMC | monocytes |
| | T-cells |
| | NK-cells |
| | dendritic cells |
| Small intestine | proximal enterocytes |
| | undifferentiated cells |
| | intestinal goblet cells |
| | Paneth cells |
| Spleen | B-cells |
| | T-cells |
| | plasma cells |
| | macrophages |
| Stomach | B-cells |
| | T-cells |
| | gastric mucus-secreting cells |
| | plasma cells |
| | fibroblasts |
| | macrophages |
| Testis | Leydig cells |
| | late spermatids |
| | spermatogonia |
| | early spermatids |
| | macrophages |
| | spermatocytes |
| | peritubular cells |
| | Sertoli cells |
| | endothelial cells |
| Peripheral nervous | motor neurons |
| system | sensory neurons |
| | Schwann cells |
| | dorsal root ganglion |
| Bone/cartilage/joint | chondrocytes |
| | chondroblasts |
| | mesenchymal cells |
| | osteoblasts |
| | osteoclasts |

### Molecular Cargoes

In some aspects, the present disclosure includes methods and compositions for delivering a conjugated molecular cargo to a cell or tissue. In certain aspects the antigen-binding protein that binds specifically to transferrin receptor (TfR) disclosed herein, e.g., an scFv, an antibody or an antigen-binding fragment thereof, may be conjugated (e.g., covalently conjugated) to the molecular cargo.

As used herein, the term “molecular cargo” refers to a molecule that operates to effect a biological outcome. As a non-limiting example, the molecular cargo may operate to modulate the transcription of a DNA sequence, to modulate the expression of a protein, or to modulate the activity of a protein, to delete or disrupt an endogenous gene (or fragment thereof), to insert an exogenous gene (or fragment thereof), or to replace an endogenous gene (or fragment thereof) with an exogenous gene (or fragment thereof). In various embodiments, the molecular cargo may comprise a polynucleotide. In various embodiments, the molecular cargo comprises a lipid nanoparticle, liposome, or non-lipid nanoparticle described herein, which optionally comprises one or more polynucleotide and/or a protein molecules. In various embodiments, the molecular cargo may comprise a small molecule.

In some embodiments, the anti-TfR antibody or an antigen-binding fragment thereof disclosed herein may be used, for example, to deliver the conjugated molecular cargo to a cell or a tissue that expresses TfR1 (e.g., the brain or the muscle) for diagnosing and or treating a disease (e.g., a neurological disease or muscular disease). In some embodiments, the molecular cargoes conjugated to the anti-TfR antibody or antigen-binding fragment thereof may be taken up by, e.g., endothelial cells, via binding to the transferrin receptor, which may be endocytosed, e.g., via clathrin-mediated endocytosis. In some embodiments, the anti-TfR antibody or an antigen-binding fragment thereof described herein can exhibit superior activity, e.g., in delivering a molecular cargo into a target tissue (e.g., brain, spinal cord, muscle, spleen, heart, or lung) or a target cell (e.g., a brain cell, or a myocyte). In some embodiments, the anti-TfR antibody or an antigen-binding fragment thereof may be effective in delivering a molecular cargo into one or more brain cells, such as a neuron (e.g., motor neuron, sensory neuron), an astrocyte, a glial cell (e.g., oligodendrocytes, microglia), and/or other cells in the brain and/or spinal cord.

In some embodiments, the molecular cargo comprises a polynucleotide molecule. The terms “polynucleotide” and “nucleic acid” are used interchangeably herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with optional substitutions, e.g., methoxy or 2′ halide substitutions. In some embodiments, polynucleotides up to about 30 nucleotides in length can be referred to herein as an “oligonucleotide”. Oligonucleotides may be of a variety of different lengths, e.g., depending on the form. In some embodiments, an oligonucleotide is 7, 8, 9, 10, 11, 12, 13, 14, 15, 16, 17, 18, 19, 20, 21, 22, 23, 24, 25, 26, 27, 28, 29, 30, or more nucleotides in length. In some embodiments, the oligonucleotide is 8 to 30 nucleotides in length, 10 to 15 nucleotides in length, 10 to 20 nucleotides in length, 15 to 25 nucleotides in length, 21 to 23 nucleotides in lengths.

In some embodiments, the molecular cargo described herein may comprise a carrier, such as a liposome or lipid nanoparticle (LNP). A lipid particle, e.g., a liposome or lipid nanoparticle disclosed herein, may include a lipid formulation that can be used to deliver a therapeutic nucleic acid (e.g., gRNA) to a target site of interest (e.g., cell, tissue, organ, and the like). Without wishing to be bound by theory, carriers may be used, e.g., as a means for delivery of a polynucleotide disclosed herein and/or a protein disclosed herein. In some embodiments, a carrier (e.g., liposome or LNP) may be useful for the delivery of a nucleic acid (e.g., DNA or RNA), protein (e.g., RNA-guided DNA binding agent), or a combination thereof. By way of a non-limiting example, a carrier (e.g., liposome or LNP) may be used to deliver various components of a gene editing system, for example, a CRISPR/Cas system or additional gene editing systems described herein.

In some embodiments, the molecular cargo comprises a small molecule. A small molecule (SM) can entercells easily because it has a low molecularweight (typically, up to about 1 kDa). Once inside the cells, it can affect other molecules, such as proteins, and may, for example, cause cancer cells to die. This is different from many large molecular weight molecules such as antibodies. An example, of a small molecule that may be conjugated to an anti-TfR antigen-binding protein, to form an anti-TfR:SM conjugate. Moreover, anti-cancer SMs may be delivered by way of anti-TfR-mediated delivery. Such anti-cancer SMs can include, for example, cytotoxic agents, alkylating agents (e.g., platinum containing drugs), antimetabolites (5-fluorouracil), topoisomerases (e.g., topotecan), anthracyclines (e.g., doxorubicin), and plant alkaloids (e.g., vinblastine). Other small molecule cargos may include Miglustat.

Exemplary molecular cargoes are described in further detail herein, however, it should be appreciated that the exemplary molecular cargoes provided herein are not intended to be limiting.

### Polynucleotide Molecules

Non-limiting examples of polynucleotide molecules that are useful as molecular cargoes in the protein-drug conjugates described herein include, but are not limited to, interfering nucleic acids (e.g., shRNAs, siRNAs, microRNAs, antisense oligonucleotides), gapmers, mixmers, ribozymes, phosphorodiamidite morpholinos, peptide nucleic acids, aptamers, and guide nucleic acids (e.g., Cas9 guide RNAs), mRNAs, etc. In various embodiments, a polynucleotide may comprise one or more modified nucleotides. In various embodiments, a polynucleotide may comprise one or more modified inter-nucleotide linkage. Polynucleotides may be single-stranded or double-stranded.

In some embodiments, the molecular cargo comprises at least one polynucleotide molecule. In some embodiments, the molecular cargo comprises at least 2, at least 3, at least 4, at least 5, or at least 10 polynucleotide molecules.

In some embodiments, the polynucleotide molecule is DNA. In some embodiments, the polynucleotide molecule is RNA.

In various embodiments, a polynucleotide described herein (e.g., interfering nucleic acid or guide RNA) may comprise a region of complementarity to a target nucleic acid which can be in the range of 8 to 15, 8 to 30, 8 to 40, or 10 to 50, or 5 to 50, or 5 to 40 nucleotides in length. In certain embodiments, a region of complementarity of a polynucleotide to a target nucleic acid may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity may be complementary with at least 10 consecutive nucleotides of a target nucleic acid. In some embodiments, a polynucleotide may contain 1, 2, 3, 4 or 5 base mismatches compared to the portion of the consecutive nucleotides of target nucleic acid. In some embodiments the polynucleotide may have up to 3 mismatches over 15 bases, or up to 4 mismatches over 10 bases. In some embodiments, the polynucleotide is complementary (e.g., at least 80%, at least 85% at least 90%, at least 95%, or 100%) to a target sequence of any one of the polynucleotides described herein. In various embodiments, such target sequence may be 100% complementary to the polynucleotide described herein. In some embodiments, any one or more of the thymine bases (T's) in any one of the polynucleotides described herein may be uracil bases (U's), and/or any one or more of the U's may be T's. A target sequence described herein may comprise a sequence of nucleic acid in a target gene that has complementarity to the guide sequence of the gRNA. The interaction of the target sequence and the guide sequence directs an RNA-guided DNA-binding agent (e.g., Cas protein) to bind, and potentially nick or cleave (depending on the activity of the agent), within the target sequence.

The polynucleotides described herein may be modified, e.g., comprise a modified nucleotide, a modified internucleoside linkage, and/or a modified sugar moiety, or combinations thereof. In addition, polynucleotides can possess one or more of the following properties: have improved cell uptake compared to unmodified polynucleotides; are not toxic to cells or mammals are not immune stimulatory; avoid pattern recognition receptors do not mediate alternative splicing; are nuclease resistant; have improved endosomal exit internally in a cell; or minimizes TLR stimulation. Any of the various modified chemistries or formats of polynucleotides disclosed herein may be combined with together. As a non-limiting example, one, two, three, four, five, six, seven, eight or more different types of modifications may be included within the same polynucleotide.

In various embodiments, particular nucleotide modification(s) may be used that render a polynucleotide into which the modification(s) are incorporated more resistant to nuclease digestion than the native oligoribonucleotide or oligodeoxynucleotide molecules, such modified polynucleotides survive intact for a longer time than unmodified polynucleotides. Exemplary modified polynucleotides include those comprising modified backbones, for example, modified internucleoside linkages such as, methyl phosphonates, phosphotriesters, phosphorothioates short chain alkyl or cycloalkyl intersugar linkages heterocyclic intersugar linkages or short chain heteroatomic or. As such, polynucleotides described herein may be stabilized against nucleolytic degradation, e.g., via incorporation of a modification, e.g., a nucleotide modification.

In various embodiments, a polynucleotide may be of up to 50 nucleotides in length in which 2 to 10, 2 to 15, 2 to 16, 2 to 17, 2 to 18, 2 to 19, 2 to 20, 2 to 25, 2 to 30, 2 to 40, or 2 to 45, nucleotides of the polynucleotide may be modified nucleotides. The polynucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the polynucleotide can be modified nucleotides. In some embodiments, the polynucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the polynucleotide are modified nucleotides. In some embodiments, the polynucleotides can have every nucleotide except 1, 2, 3, 4, 5, 6, 7, 8, 9, 10, 11, 12, 13, 14, or 15 nucleotides modified.

In various embodiments, the polynucleotide disclosed herein may comprise at least one nucleoside, e.g., modified at the 2′ position of the sugar. In some embodiments, all of the nucleosides in the polynucleotide are 2′-modified nucleosides. In some embodiments, a polynucleotide comprises at least one 2′-modified nucleoside.

In various embodiments, the polynucleotide disclosed herein may one or more non-bicyclic 2′-modified nucleosides, e.g., 2′-O-dimethylaminoethyloxyethyl (2′-O-DMAEOE)2′-O-methyl (2′-O-Me), 2′-O-dimethylaminoethyl (2′-O-DMAOE), 2′-O-methoxyethyl (2′-MOE), 2′-deoxy, 2′-O—N-methylacetamido (2′-O-NMA) modified nucleoside, 2′-fluoro (2′-F), 2′-O-aminopropyl (2′-O-AP), or 2′-O-dimethylaminopropyl (2′-O-DMAP).

In some embodiments, the polynucleotide described herein may comprise one or more 2′-4′ bicyclic nucleosides in which the ribose ring may comprise a bridge moiety, e.g., connecting two atoms in the ring (e.g., connecting the 2′-O atom to the 4′-C atom via an ethylene (ENA) bridge, a methylene (LNA) bridge, or a (S)-constrained ethyl (cEt) bridge). Non-limiting examples of ENAs are disclosed in PCT Publication No. WO 2005/042777; Morita et al., Nucleic Acid Res., Suppl 1:241-242, 2001; Koizumi, Curr. Opin. Mol. Ther., 8:144-149, 2006, Surono et al., Hum. Gene Ther., 15:749-757, 2004; and Horie et al., Nucleic Acids Symp. Ser (Oxf), 49:171-172, 2005; the disclosures of which are incorporated herein by reference in their entireties. Non-limiting examples of LNAs are disclosed in PCT Patent Application Publication No. WO2008/043753, the contents of which are incorporated herein by reference in its entirety. Non-limiting examples of cEt are disclosed in in U.S. Pat. Nos. 7,569,686, 7,101,993, and 7,399,845 each of which is herein incorporated by reference in its entirety.

In various embodiments, the polynucleotide described herein may comprise a modified nucleoside disclosed in, for example, U.S. Pat. Nos. 8,022,193; 7,569,686; 7,399,845; 7,741,457; 7,335,765; 7,816,333; 8,957,201; 7,314,923, the entire contents of each of which are incorporated herein by reference for all purposes.

In various embodiments, the polynucleotide comprises at least one modified nucleoside that results in an increase in Tm of the polynucleotide in a range of 1° C. to 10° C. compared with a polynucleotide that does not have the at least one modified nucleoside. The polynucleotide may have a plurality of modified nucleosides that result in a total increase in Tm of the polynucleotide in a range of 2° C., 3° C., 4° C., 5° C., 6° C., 7° C., 8° C., 9° C., 10° C., 15° C., 20° C., 25° C., 30° C., 35° C., 40° C., 45° C., 50° C., 55° C., 60° C. or more as compared to a polynucleotide which does not have the modified nucleoside.

In some embodiments, the polynucleotide may comprise a mix of nucleosides of different kinds. A polynucleotide may comprise a mix of deoxyribonucleosides or ribonucleosides and 2′-O-Me modified nucleosides. A polynucleotide may comprise a mix of 2′-4′ bicyclic nucleosides and 2′-MOE, 2′-fluoro, or 2′-O-Me modified nucleosides. A polynucleotide may comprise a mix of non-bicyclic 2′-modified nucleosides (e.g., 2′-MOE, 2′-fluoro, or 2′-O-Me) and 2′-4′ bicyclic nucleosides (e.g., LNA, ENA, cEt). A polynucleotide may comprise a mix of 2′-deoxyribonucleosides or ribonucleosides and 2′-fluoro modified nucleosides. A polynucleotide may comprise a mix of 2′-fluoro modified nucleosides and 2′-O-Me modified nucleosides.

In various embodiments, the oligonucleotide may comprise alternating nucleosides of different types. In certain embodiments, the oligonucleotide may comprise alternating deoxyribonucleosides or ribonucleosides and 2′-O-Me modified nucleosides. In certain embodiments, a polynucleotide may comprise alternating 2′-deoxyribonucleosides or ribonucleosides and 2′-fluoro modified nucleosides. In certain embodiments, the oligonucleotide may comprise alternating 2′-fluoro modified nucleosides and 2′-O-Me modified nucleosides. In certain embodiments, the oligonucleotide may comprise alternating 2′-4′ bicyclic nucleosides and 2′-MOE, 2′-fluoro, or 2′-O-Me modified nucleosides. In certain embodiments, the oligonucleotide may comprise alternating non-bicyclic 2′-modified nucleosides (e.g., 2′-MOE, 2′-fluoro, or 2′-O-Me) and 2′-4′ bicyclic nucleosides (e.g., LNA, ENA, cEt).

In various embodiments, a polynucleotide described herein may comprise one or more abasic residues, a 5-vinylphosphonate modification, and/or one or more inverted abasic residues.

In various embodiments, the oligonucleotide may comprise a phosphorothioate or other modified internucleoside linkage. In various embodiments, the oligonucleotide may comprise phosphorothioate internucleoside linkages. In various embodiments, the oligonucleotide comprises phosphorothioate internucleoside linkages between at least two nucleotides. In various embodiments, the oligonucleotide comprises phosphorothioate internucleoside linkages between all nucleotides. By way of a non-limiting example, in certain embodiments, oligonucleotides comprise modified internucleoside linkages at the first, second, and/or (e.g., and) third internucleoside linkage at the 5′ or 3′ end of the nucleotide sequence.

Non-limiting examples of phosphorus-containing linkages include aminoalkylphosphotriesters phosphorothioates, chiral phosphorothioates, phosphotriesters, phosphorodithioates, methyl and other alkyl phosphonates comprising 3′alkylene phosphonates and chiral phosphonates, phosphinates, phosphoramidates comprising 3-amino phosphoramidate and aminoalkylphosphoramidates, thionoalkylphosphonates, thionophosphoramidates, thionoalkylphosphotriesters, and boranophosphates having normal 3-5′ linkages, 2′-5′ linked analogs of these, and those having inverted polarity wherein the adjacent pairs of nucleoside units are linked 3-5′ to 5′-3′ or 2′-5′ to 5′-2′; see U.S. Pat. Nos. 5,625,050; 4,469,863; 4,476,301; 5,023,243; 5,550,111; 5,177,196; 5,587,361; 5,188,897; 5,264,423; 5,276,019; 5,519,126; 5,278,302; 5,286,717; 5,321,131; 5,399,676; 5,405,939; 5,453,496; 5,455, 233; 5,466,677; 5,476,925; 5,536,821; 5,541,306; 5,563, 253; 5,571,799; and 3,687,808.

In various embodiments, a polynucleotide described herein may have heteroatom backbones, e.g., or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497), morpholino backbones (see Summerton and Weller, U.S. Pat. No. 5,034,506); amide backbones (see De Mesmaeker et al. Ace. Chem. Res. 1995, 28:366-374); or MMI or methylene(methylimino) backbones.

Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 6-O -methylguanine, 4-thio-pyrimidines, 4-amino-pyrimidines, 4-dimethylhydrazine-pyrimidines, and 4-O-alkyl-pyrimidines; U.S. Pat. No. 5,378,825 and PCT Publication No. WO 93/13121). For general discussion see Adams et al, The Biochemistry of the Nucleic Acids 5-36, 11th ed., 1992. Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional nucleosides with 2′ methoxy substituents, or polymers containing both conventional nucleotides and one or more nucleotide analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42): 13233-41). RNA and DNA have different sugar moieties and can differ by the presence of uracil or analogs thereof in RNA and thymine or analogs thereof in DNA.

### Interfering Nucleic Acids

In some embodiments, a conjugated molecular cargo may comprise a polynucleotide molecule(s) which is capable of modifying expression of one or more genes (e.g., inhibiting gene expression and/or translation, modulating RNA splicing or inducing exon skipping) in a target cell. In some embodiments, the polynucleotide molecule may be an interfering nucleic acid molecule, e.g., an siRNA, an shRNA, a miRNA, or an antisense oligonucleotide (ASO), that targets, e.g., an RNA (e.g., an mRNA).

In certain embodiments, interfering nucleic acid molecules that selectively target and inhibit the activity or expression of a product (e.g., an mRNA product) of a targeted gene are used in compositions and methods described herein. An interfering nucleic acid molecule may inhibit the expression or activity of a product (e.g., an mRNA product) of at least one targeted gene by at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, or 100%. An agent disclosed herein may comprise a nucleobase sequence that is at least 80%, at least 85%, at least 90%, at least 95%, or 100% complementarity to a product (e.g., an mRNA product) of at least targeted gene. Without wishing to be bound by theory, “complementarity” of nucleic acids can mean that a nucleotide sequence in one strand of nucleic acid, due to orientation of its nucleobase groups, forms hydrogen bonds with another sequence on an opposing nucleic acid strand. The complementary bases in DNA are typically A with T and C with G. In RNA, they are typically C with G and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm (melting temperature) of hybridized strands, or by empirical determination of Tm by using routine methods. Tm includes the temperature at which a population of hybridization complexes formed between two nucleic acid strands are 50% denatured (i.e., a population of double-stranded nucleic acid molecules becomes half dissociated into single strands). At a temperature below the Tm, formation of a hybridization complex is favored, whereas at a temperature above the Tm, melting or separation of the strands in the hybridization complex is favored. Tm may be estimated for a nucleic acid having a known G+C content in an aqueous 1 M NaCl solution by using, e.g., Tm=81.5+0.41 (% G+C), although other known Tm computations take into account nucleic acid structural characteristics.

Interfering nucleic acids can include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence.

Typically, at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acid molecule is single-stranded RNA. In some embodiments, the interfering nucleic acid molecule is double-stranded RNA. The double-stranded RNA molecule may have a 1-3 nucleotide 3′ and/or 5′ overhang in either a sense strand and/or an antisense strand. In some embodiments, the double-stranded RNA molecule has a 2 nucleotide 3′ overhang. In some embodiments, the two RNA strands are connected via a hairpin structure, forming a shRNA molecule. shRNA molecules can contain hairpins derived from microRNA molecules.

Interfering nucleic acid molecules described herein can contain RNA bases, non-RNA bases or a mixture of RNA bases and non-RNA bases. For example, interfering nucleic acid molecules described herein can be primarily composed of RNA bases or modified RNA bases, but also contain DNA bases, modified DNA bases, and/or non-naturally occurring nucleotides. The term “ribonucleotide” or “nucleotide” can, in the case of a modified RNA or nucleotide surrogate, also refer to a modified nucleotide, or surrogate replacement moiety at one or more positions.

In some embodiments, the interfering nucleic acid molecule is a small interfering RNAs (siRNA), also known as short interfering RNA or silencing RNA. siRNAs are a class of double-stranded RNA molecules, typically about 20-25 base pairs in length that target nucleic acids (e.g., mRNAs) for degradation via the RNA interference (RNAi) pathway in cells. Such siRNA molecules typically include a region of sufficient homology to the target region, and are of sufficient length in terms of nucleotides, such that the siRNA molecules down-regulate target nucleic acid. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

Specificity of siRNA molecules may be measured via the binding of the antisense strand of the molecule to its target RNA. Effective siRNA molecules are often fewer than 30 to 35 base pairs in length, e.g., to prevent stimulation of non-specific RNA interference pathways in the cell by way of the interferon response, however longer siRNA may also be effective. In various embodiments, the siRNA molecules are 5, 6, 7, 8, 9, 10, 11, 12, 13, 14, 15, 16, 17, 18, 19, 20, 21, 22, 23, 24, 25, 26, 27, 28, 29, or30 base pairs in length. In various embodiments, the siRNA molecules are about 35 to about 70 more base pairs in length. In some embodiments, the siRNA molecules are more than 70 base pairs in length. In some embodiments, the siRNA molecules are 8 to 40 base pairs in length, 10 to 20 base pairs in length, 10 to 30 base pairs in length, 15 to 20 base pairs in length, 19 to 23 base pairs in length, 21 to 24 base pairs in length. In some embodiments, the sense and antisense strands of the siRNA molecules are each independently about 19 to about 24 nucleotides in length. In some embodiments, the sense strand of an siRNA molecule is 23 nucleotides in length and the antisense strand is 21 nucleotides in length. In some embodiments, both the sense strand and the antisense strand of an siRNA molecule are 21 nucleotides in length.

After selection of a suitable target RNA sequence, siRNA molecules that comprise a nucleotide sequence complementary to all or a portion of the target sequence, i.e., an antisense sequence, may be designed and prepared using suitable methods (see, e.g., U.S. Patent Publication Nos. 2004/0077574 and 2008/0081791 and PCT Publication No. WO 2004/016735). In some embodiments, the siRNA molecule may be single-stranded (i.e. a ssRNA molecule comprising just an antisense strand) or double stranded (i.e. a dsRNA molecule comprising an antisense strand and a complementary sense strand that hybridizes to form the dsRNA). In various embodiments, the siRNA molecules may comprise a duplex, asymmetric duplex, hairpin or asymmetric hairpin secondary structure, comprising self-complementary sense and/or antisense strands.

In various embodiments, the antisense strand of the siRNA molecule is 5, 6, 7, 8, 9, 10, 11, 12, 13, 14, 15, 16, 17, 18, 19, 20, 21, 22, 23, 24, 25, 26, 27, 28, 29, or 30 nucleotides in length. In various embodiment, the antisense strand of the siRNA molecule is about 35 to about 70 nucleotides in length. In various embodiment, the antisense strand of the siRNA molecule is more than 70 nucleotides in length. In some embodiments, the antisense strand is 8 to 40 nucleotides in length, 10 to 20 nucleotides in length, 10 to 30 nucleotides in length, 15 to 20 nucleotides in length, 19 to 23 nucleotides in length, or 21 to 24 nucleotides in length.

In some embodiments, the sense strand of the siRNA molecule is 5, 6, 7, 8, 9, 10, 11, 12, 13, 14, 15, 16, 17, 18, 19, 20, 21, 22, 23, 24, 25, 26, 27, 28, 29, or 30 nucleotides in length. In various embodiments, the sense strand of the siRNA molecule is about 30 to about 70 nucleotides in length. In various embodiments, the sense strand of the siRNA molecule more than 70 nucleotides in length. In some embodiments, the sense strand is 8 to 40 nucleotides in length, 10 to 20 nucleotides in length, 10 to 30 nucleotides in length, 15 to 20 nucleotides in length, 19 to 23 nucleotides in length, 21 to 24 nucleotides in length.

In various embodiments, siRNA molecules can comprise an antisense strand comprising a region of complementarity to a target region in a target mRNA. In some embodiments, the region of complementarity is at least 70%, at least 75%, at least 80%, at least 85%, at least 90%, at least 91%, at least 92%, at least 93%, at least 94%, at least 95%, at least 96%, at least 97%, at least 98%, at least 99% or 100% complementary to a target region in a target mRNA. In some embodiments, the target region may comprise a region of consecutive nucleotides in the target mRNA. In some embodiments, it may not be requisite for a region of complementarity to be 100% complementary to that of its target to be specifically hybridizable or specific for a target RNA sequence.

In some embodiments, siRNA molecules disclosed herein may comprise an antisense strand that comprises a region of complementarity to a target RNA sequence and the region of complementarity is in the range of 8 to 20, 8 to 35, 8 to 45, or 10 to 50, or 5 to 55, or 5 to 40 nucleotides in length. In some embodiments, a region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, or more consecutive nucleotides of a target RNA sequence. In some embodiments, siRNA molecules comprise an antisense strand having a nucleotide sequence that contains no more than 1, 2, 3, 4, 5, 6, 7, 8, 9, or 10 base mismatches compared to the portion of the consecutive nucleotides of target RNA sequence. In some embodiments, siRNA molecules comprise a nucleotide sequence that has up to 3 mismatches over 15 bases, or up to 4 mismatches over 10 bases with a target sequence.

In some embodiments, siRNA molecules comprises an antisense strand having a nucleotide sequence that has up 0, 1, 2, or 3 mismatches over 15-22 bases with a target sequence. In some embodiments, siRNA molecules comprises an antisense strand having a nucleotide sequence that has 0, 1, or 2 mismatches over 15-22 bases with a target sequence. In some embodiments, siRNA molecules comprises an antisense strand having a nucleotide sequence that has 0 or 1 mismatch over 15-22 bases with a target sequence. In some embodiments, siRNA molecules comprises an antisense strand having a nucleotide sequence that has 0 mismatches over 15-22 bases with a target sequence.

In various embodiments, siRNA molecules may comprise an antisense strand comprising a nucleotide sequence that is at least 70%, at least 75%, at least 85%, at least 90%, at least 95%, or 100% complementary to the target RNA sequence of the antisense oligonucleotides disclosed herein. In some embodiments, siRNA molecules comprise an antisense strand comprising a nucleotide sequence that is at least 70%, at least 75%, at least 85%, at least 90%, at least 95%, or 100% identical to any of the antisense oligonucleotides provided herein. In some embodiments, siRNA molecules comprise an antisense strand comprising at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, or more consecutive nucleotides of any of the antisense oligonucleotides provided herein.

In some embodiments, double-stranded siRNA can comprise sense and anti-sense RNA strands that are different lengths or the same length. In some embodiments, double-stranded siRNA molecules may also be generated from a single oligonucleotide in a stem-loop structure. The self-complementary sense and antisense regions of the siRNA molecule having a stem-loop structure may be linked by means of a nucleic acid based or a non-nucleic acid-based linker. In some embodiments, an siRNA having a stem-loop structure comprises a circular single-stranded RNA having two or more loop structures and a stem comprising self-complementary sense and antisense strands. In some embodiments, the circular RNA may be processed in vivo or in vitro to produce an active siRNA molecule which may be capable of mediating RNAi. Small hairpin RNA (shRNA) molecules are therefore also contemplated herein. Such molecules may comprise a specific antisense sequence together with the reverse complement (sense) sequence, which may be separated by a spacer or loop sequence in some instances. A reverse complement described herein may comprise a sequence that is a complement sequence of a reference sequence, wherein the complement sequence is written in the reverse orientation. Due to codon usage redundancy, a reverse complement can diverge from a reference sequence that encodes the same polypeptide. As used herein, “reverse complement” also includes sequences that are, e.g., at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the reverse complement sequence of a reference sequence. Cleavage of the spacer or loop can provide a single-stranded RNA molecule and its reverse complement, such that they may anneal to form a dsRNA molecule. In various embodiments, additional optional processing steps may result in removal or addition of 1, 2, 3, 4, 5 or more nucleotides from the 3′ end and/or the 5′ end of one or both strands. A spacer may be of a suitable length to allow the antisense and sense sequences to anneal and form a double-stranded structure or stem prior to cleavage of the spacer. In certain embodiments subsequent optional processing steps may result in removal or addition of 1, 2, 3, 4, 5 or more nucleotides from the 3′ end and/or the 5′ end of one or both strands. In some embodiments, a spacer sequence can be an unrelated nucleotide sequence that may be, e.g., situated between two complementary nucleotide sequence regions that, when annealed into a double-stranded nucleic acid, can comprise a shRNA.

The length of the siRNA molecules can vary from about 10 to about 120 nucleotides depending on the type of siRNA molecule being designed. Generally, between about 10 and about 55 of these nucleotides may be complementary to the RNA target sequence. For instance, when the siRNA is a double-stranded siRNA or single-stranded siRNA, the length can vary from about 10 to about 55 nucleotides, whereas when the siRNA is a shRNA or circular molecule, the length can vary from about 30 nucleotides to about 110 nucleotides.

In various embodiments, an siRNA molecule can comprise a 3 overhang at one end of the molecule. In some embodiments, the other end can be blunt-ended or may also comprise an overhang (e.g., 5′ and/or 3). When the siRNA molecule comprises an overhang at both ends of the molecule, the length of the overhangs may be different or the same. In some embodiments, an siRNA molecule described herein may comprises 3′ overhangs of about 1 to about 3 nucleotides on both ends of the molecule. In some embodiments, the siRNA molecule comprises 3′ overhangs of about 1 to about 3 nucleotides on both the sense strand and the antisense strand. In some embodiments, the siRNA molecule comprises 3′ overhangs of about 1 to about 3 nucleotides on the antisense strand. In some embodiments, the siRNA molecule may comprise 3′ overhangs of about 1 to about 3 nucleotides on the sense strand.

In various embodiments, the siRNA molecule comprises one or more modified nucleotides (e.g., 1, 2, 3, 4, 5, 6, 7, 8, 9, 10, 11, 12, 13, 14, 15 or more). In some embodiments, all of the nucleotides of the sense strand and/or the antisense strand of the siRNA molecule are modified. In certain embodiments, the siRNA molecule can comprise one or more modified nucleotides and/or one or more modified internucleotide linkages. In some embodiments, the siRNA molecule may comprise modified internucleotide linkages at the first and second internucleoside linkages at the 5′ end of the siRNA molecule sense strand. In some embodiments, the siRNA molecule may comprise modified internucleotide linkages at the first and second internucleoside linkages at the 5′ and 3′ ends of the siRNA molecule antisense strand. In some embodiments, the siRNA molecule may comprise modified internucleotide linkages at the first and second internucleoside linkages at the 5′ end of the siRNA molecule sense strand and at the first and second internucleoside linkages at the 5′ and 3′ ends of the siRNA molecule antisense strand.

In some embodiments, the modified nucleotide may comprise a modified sugar moiety (e.g., a 2′ modified nucleotide). In some embodiments, the siRNA molecule can comprise one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In various embodiments, each nucleotide of the siRNA molecule can a modified nucleotide (e.g., a 2′-modified nucleotide). In some embodiments, the siRNA molecule may comprise one or more phosphorodiamidate morpholinos. In some embodiments, each nucleotide of the siRNA molecule consists of a phosphorodiamidate morpholino.

In various embodiments, the siRNA molecule may comprise a phosphorothioate or other modified internucleotide linkage. In various embodiments, the siRNA molecule may comprise, e.g., a phosphorothioate internucleoside linkage(s). In some embodiments, the siRNA molecule may comprise a phosphorothioate internucleoside linkage(s) between two or more nucleotides. In some embodiments, the siRNA molecule may comprise a phosphorothioate internucleoside linkage(s) between all nucleotides. In some embodiments, the siRNA molecule may comprise modified internucleotide linkages at the first, second, and/or third internucleoside linkage at the 5′ or 3′ end of the siRNA molecule. In some embodiments, the siRNA molecule may comprise modified internucleotide linkages at the first and second internucleoside linkages at the 5′ and/or 3′ end of the siRNA molecule. In some embodiments, the siRNA molecule may comprise modified internucleotide linkages at the first and second internucleoside linkages at the 5′ end of the siRNA molecule sense strand. In some embodiments, the siRNA molecule may comprise modified internucleotide linkages at the first and second internucleoside linkages at the 5′ and 3′ ends of the siRNA molecule antisense strand. In some embodiments, the siRNA molecule may comprise modified internucleotide linkages at the first and second internucleoside linkages at the 5′ end of the siRNA molecule sense strand and at the first and second internucleoside linkages at the 5′ and 3′ ends of the siRNA molecule antisense strand. In some embodiments, the siRNA molecule may comprise modified internucleotide linkages at the first internucleoside linkage at the 5′ and 3′ ends of the siRNA molecule sense strand, at the first, second, and third internucleoside linkages at the 5′ end of the siRNA molecule antisense strand, and at the first internucleoside linkage at the 3′ end of the siRNA molecule antisense strand.

In various embodiments, the modified internucleotide linkages may comprise phosphorus-containing linkages. In some embodiments, phosphorus-containing linkages which may be used in the methods or compositions described herein include, without limitation, chiral phosphorothioates, phosphorothioates, phosphorodithioates, aminoalkylphosphotriesters, phosphotriesters, methyl and other alkyl phosphonates comprising 3′alkylene phosphonates and chiral phosphonates, phosphoramidates comprising 3′-amino phosphoramidate and aminoalkylphosphoramidates, phosphinates, thionoalkylphosphonates, thionophosphoramidates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 5,625,050; 3,687,808; 4,469,863; 4,476,301; 5,177,196; 5,455, 233; 5,264,423; 5,276,019; 5,278,302; 5,286,717; 5,321,131; 5,399,676; 5,405,939; 5,519,126; 5,453,496; 5,466,677; 5,476,925; 5,536,821; 5,023,243; 5,541,306; 5,550,111; 5,563, 253; 5,571,799; 5,587,361; and 5,188,897.

Any of the various modified formats or chemistries of siRNA molecules disclosed herein may be combined together. For example, without limitation, 1, 2, 3, 4, 5, 6, 7, 8, 9, or 10 or more different types of modifications may be included within the same siRNA molecule.

In various embodiments, the antisense strand may comprise one or more modified nucleotides (e.g., 1, 2, 3, 4, 5, 6, 7, 8, 9, 10, 11, 12, 13, 14, 15 or more). In some embodiments, the antisense strand may comprise one or more modified nucleotides and/or one or more modified internucleotide linkage(s). In some embodiments, the modified nucleotide may comprise a modified sugar moiety (e.g., a 2′ modified nucleotide). In some embodiments, the antisense strand comprises one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In various embodiments, each nucleotide of the antisense strand can be a modified nucleotide (e.g., a 2′-modified nucleotide). In some embodiments, the antisense strand may comprise one or more phosphorodiamidate morpholinos. In some embodiments, the antisense strand consists of a phosphorodiamidate morpholino oligomer (PMO).

In some embodiments, antisense strand contains a phosphorothioate or other modified internucleotide linkage. In some embodiments, the antisense strand may comprise phosphorothioate internucleoside linkage(s). In some embodiments, the antisense strand may comprise phosphorothioate internucleoside linkage(s) between two or more nucleotides. In some embodiments, the antisense strand may comprise phosphorothioate internucleoside linkage(s) between all nucleotides. In some embodiments, the antisense strand may comprise modified internucleotide linkages at the first, second, and/or third nucleotide at the 5′ or 3′ end of the antisense strand. In some embodiments, the antisense strand may comprise modified internucleotide linkages at the first and second nucleotide positions (e.g., between the first and second and between the second and third nucleotides) at the 5′ and 3′ ends of the antisense strand.

In various embodiments, the modified internucleotide linkages may comprise phosphorus-containing linkages of the antisense strand. In some embodiments, phosphorus-containing linkages which may be used in methods and compositions described herein include, without limitation, chiral phosphorothioates, phosphorothioates, phosphorodithioates, aminoalkylphosphotriesters, phosphotriesters, methyl and other alkyl phosphonates comprising 3′alkylene phosphonates and chiral phosphonates, phosphoramidates comprising 3′-amino phosphoramidate and aminoalkylphosphoramidates, phosphinates, thionoalkylphosphonates, thionophosphoramidates, thionoalkylphosphotriesters, and boranophosphates having normal 3-5′ linkages, 2′-5′ linked analogs of these, and those having inverted polarity wherein the adjacent pairs of nucleoside units are linked 3-5′ to 5′-3′ or 2′-5′ to 5′-2′; see U.S. Pat. Nos. 5,625,050; 3,687,808; 4,469,863; 4,476,301; 5,177,196; 5,455, 233; 5,264,423; 5,276,019; 5,278,302; 5,286,717; 5,321,131; 5,399,676; 5,405,939; 5,519,126; 5,453,496; 5,466,677; 5,476,925; 5,536,821; 5,023,243; 5,541,306; 5,550,111; 5,563, 253; 5,571,799; 5,587,361; and 5,188,897.

Any of the modified formats or chemistries of the antisense strand disclosed herein may be combined together. For example, without limitation, 1, 2, 3, 4, 5, 6, 7, 8, 9, or 10 or more different types of modifications may be included within the same antisense strand.

In some embodiments, the sense strand comprises one or more modified nucleotides (e.g., 1, 2, 3, 4, 5, 6, 7, 8, 9, 11, 12, 13, 14, 15 or more). In some embodiments, the antisense strand may comprise one or more modified nucleotides and/or one or more modified internucleotide linkage(s). In some embodiments, the modified nucleotide may comprise a modified sugar moiety (e.g., a 2′ modified nucleotide). In some embodiments, the antisense strand comprises one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In various embodiments, each nucleotide of the antisense strand can be a modified nucleotide (e.g., a 2′-modified nucleotide). In some embodiments, the antisense strand may comprise one or more phosphorodiamidate morpholinos. In some embodiments, the antisense strand consists of a phosphorodiamidate morpholino oligomer (PMO).

In some embodiments, the sense strand contains a phosphorothioate or other modified internucleotide linkage. In some embodiments, the sense strand may comprise phosphorothioate internucleoside linkage(s). In some embodiments, the sense strand may comprise phosphorothioate internucleoside linkage(s) between two or more nucleotides. In some embodiments, the sense strand may comprise phosphorothioate internucleoside linkages between all nucleotides. For example, in some embodiments, the sense strand comprises modified internucleotide linkages at the first, second, and/or third nucleotide at the 5′ or 3′ end of the sense strand. In some embodiments, the sense strand may comprise modified internucleotide linkages at the first and second nucleotide positions (e.g., between the first and second and between the second and third nucleotides) at the 5′ end of the sense strand.

In various embodiments, the modified internucleotide linkages may comprise phosphorus-containing linkages of the sense strand. In some embodiments, phosphorus-containing linkages which may be used in the methods and compositions described herein include, without limitation, chiral phosphorothioates, phosphorothioates, phosphorodithioates, aminoalkylphosphotriesters, phosphotriesters, methyl and other alkyl phosphonates comprising 3′alkylene phosphonates and chiral phosphonates, phosphoramidates comprising 3-amino phosphoramidate and aminoalkylphosphoramidates, phosphinates, thionoalkylphosphonates, thionophosphoramidates, thionoalkylphosphotriesters, and boranophosphates having normal 3-5′ linkages, 2′-5′ linked analogs of these, and those having inverted polarity wherein the adjacent pairs of nucleoside units are linked 3-5′ to 5′-3′ or 2′-5′ to 5′-2′; see U.S. Pat. Nos. 5,625,050; 3,687,808; 4,469,863; 4,476,301; 5,177,196; 5,455, 233; 5,264,423; 5,276,019; 5,278,302; 5,286,717; 5,321,131; 5,399,676; 5,405,939; 5,519,126; 5,453,496; 5,466,677; 5,476,925; 5,536,821; 5,023,243; 5,541,306; 5,550,111; 5,563, 253; 5,571,799; 5,587,361; and 5,188,897.

Any of the modified chemistries or formats of the sense strand described herein can be combined together. For example, without limitation, 1, 2, 3, 4, 5, 6, 7, 8, 9, or 10 or more different types of modifications may be included within the same sense strand.

In various embodiments, the antisense and/or sense strand of the siRNA molecule may comprise one or more modifications capable of enhancing or reducing, e.g., RNA-induced silencing complex (RISC) loading. In some embodiments, the antisense strand of the siRNA molecule may comprise one or more modifications capable of enhancing RISC loading. In various embodiments, the sense strand of the siRNA molecule may comprise one or more modifications capable of reducing RISC loading and/or reducing off-target effects. In various embodiments, the antisense strand of the siRNA molecule may comprise a 2′-O—methoxyethyl (2′-MOE) modification. In some embodiments, the addition of the 2′-O-methoxyethyl (2′-MOE) group, e.g., at the cleavage site may improve the silencing activity and/or specificity of siRNAs, e.g., by facilitating the oriented RNA-induced silencing complex (RISC) loading of the modified strand, e.g., as disclosed in Song et al., (2017) Mol Ther Nucleic Acids 9:242-250, incorporated herein by reference in its entirety. In various embodiments, the antisense strand of the siRNA molecule may comprise a 2′-O-Me-phosphorodithioate modification. In some embodiment, the 2′-O-Me-phosphorodithioate modification may increase RISC loading, e.g., as disclosed in Wu et al., (2014) Nat Commun 5:3459, incorporated herein by reference in its entirety.

In various embodiments, the sense strand of the siRNA molecule may comprise a 5′-nitroindole modification. In some embodiments, the 5′-nitroindole modification may decrease the RNAi potency of the sense strand and/or reduces off-target effects, e.g., as disclosed in Zhang et al., (2012) Chembiochem 13(13): 1940-1945, incorporated herein by reference in its entirety. In various embodiments, the sense strand may comprise a 2′-O-methyl (2′-O-Me) modification. In some embodiments, the 2′-O-Me modification may reduce RISC loading and/or the off-target effects of the sense strand, e.g., as disclosed in Zheng et al., FASEB (2013) 27(10): 4017-4026, incorporated herein by reference in its entirety. In various embodiments, the sense strand of the siRNA molecule may be fully substituted with morpholino, 2′-MOE and/or 2′-O-Me residues, and may not be recognized by RISC, e.g., as disclosed in Kole et al., (2012) Nature reviews. Drug Discovery 11(2): 125-140, incorporated herein by reference in its entirety.

In various embodiments, the sense strand of the siRNA molecule may comprise a 5′-morpholino modification. In various embodiments, the 5′-morpholino modification may reduce RISC loading of the sense strand and/or improves RNAi activity and/or antisense strand selection, e.g., as disclosed in Kumar et al., (2019) Chem Commun (Camb) 55(35):5139-5142, incorporated herein by reference in its entirety. In various embodiments, the sense strand of the siRNA molecule may be modified, for example, with a synthetic RNA-like high affinity nucleotide analogue called Locked Nucleic Acid (LNA) that may reduce RISC loading of the sense strand and promote antisense strand incorporation into RISC, e.g., as disclosed in Elman et al., (2005) Nucleic Acids Res. 33(1): 439-447, incorporated herein by reference in its entirety. In various embodiments, the sense strand of the siRNA molecule may comprise a 5′ unlocked nucleic acid (UNA) modification. In various embodiments, the 5′ unlocked nucleic acid (UNA) modification may reduce RISC loading of the sense strand and/or improve silencing capability of the antisense strand, e.g., as disclosed in Snead et al., (2013) Mol Ther Nucleic Acids 2(7):e103, incorporated herein by reference in its entirety.

In some embodiments, the antisense strand of the siRNA molecule may comprise a 2′-MOE modification and/or the sense strand may comprise an 2′-O-Me modification (see e.g., Song et al., (2017) Mol Ther Nucleic Acids 9:242-250). In some embodiments at least one (e.g., at least 2, at least 3, at least 4, at least 5, at least 6, at least 5, at least 8, at least 9, at least 10 or more) siRNA molecule may be conjugated, for example, covalently to an anti-TfR antigen-binding protein described herein. In some embodiments, the anti-TfR antigen-binding protein may be conjugated to the 5′ end of the sense strand of the siRNA molecule. In some embodiments, the anti-TfR antigen-binding protein may be conjugated to the 3′ end of the sense strand of the siRNA molecule. In some embodiments, the the anti-TfR antigen-binding protein may be conjugated internally to the sense strand of the siRNA molecule. In some embodiments, the anti-TfR antigen-binding protein may be conjugated to the 5′ end of the antisense strand of the siRNA molecule. In some embodiments, the anti-TfR antigen-binding protein may be conjugated to the 3′ end of the antisense strand of the siRNA molecule. In some embodiments, the anti-TfR antigen-binding protein be conjugated internally to the antisense strand of the siRNA molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-termini of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (e.g., C3-C12 (e.g., C3, C6, C9, C12), abasic, tri ethylene glycol, hexaethylene glycol), biotin or fluorescein reagents that come as phosphoramidites and that have another DMT-protected hydroxyl group, allowing multiple couplings during RNA synthesis.

In some embodiments, the sense strand is 23 nucleotides in length and the antisense strand is 21 nucleotides in length. In some embodiments, the sense strand is 23 nucleotides in length and the antisense strand is 21 nucleotides in length, wherein the 3′ and 5′ terminal nucleotide positions of the sense strand are inverted abasic residues. The sense strand 3′ and 5′ terminal inverted abasic residues may be overhangs. The inverted abasic residues may be linked via a 3′-3′ phosphodiester linkage. In some embodiments, the antisense strand of the siRNA molecule contains 1-2 phosphorothioate linkages at the 3′ and/or 5′ ends. In some embodiments, the antisense strand contain two or three phosphorothioate internucleotide linkages at the 5′-terminus and 1 phosphorothioate internucleotide linkage at the 3′-terminus. The siRNA molecule may be linked to a targeting moiety at the 5′ or 3′ end of the sense strand.

In some embodiments, the sense strand is 21 nucleotides in length and the antisense strand is 23 nucleotides in length, wherein the antisense strand contains a 2 nucleobase 3′ overhang. In some embodiments, the antisense strand of the siRNA molecule contains 1-3 phosphorothioate linkages at the 3′ and 5′ ends and the sense strand of the siRNA molecule contains 1-2 phosphorothioate linkages at the 5′ end. In some embodiments, the antisense strand of the siRNA molecule contains 2-3 phosphorothioate linkages at the 5′ end and 2 phosphorothioate linkages at the 3′, and the sense strand of the siRNA molecule contains 2 phosphorothioate linkages at the 5′ end. The siRNA molecule may be linked to a targeting moiety at the 5′ or 3′ end of the sense strand.

In some embodiments, the interfering nucleic acid molecule is a short hairpin RNA (shRNA). A “small hairpin RNA” or “short hairpin RNA” or “shRNA” described herein may include a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure may be cleaved by the cellular machinery into siRNA, which is then bound to the RNA-induced silencing complex (RISC).

Non-limiting examples of shRNAs include a double-stranded polynucleotide molecule assembled from a single-stranded molecule, where the sense and antisense regions are linked by a nucleic acid-based or non-nucleic acid-based linker, and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

Additional embodiments related to the shRNAs, as well as methods of designing and synthesizing such shRNAs, are described in U.S. Patent Publication No. 2011/0071208, the disclosure of which is herein incorporated by reference in its entirety for all purposes.

In some embodiments, the interfering nucleic acid molecule is a microRNA (miRNA). miRNAs represent a large group of small RNAs produced naturally in organisms, some of which regulate the expression of target genes. miRNAs are short hairpin RNAs about 18 to about 25 nucleotides in length that function in RNA silencing and post-translational regulation of gene expression. Typically, miRNAs are generated from large RNA precursors (termed pri-miRNAs) that are processed in the nucleus into approximately 70 nucleotide pre-miRNAs, which fold into imperfect stem-loop structures. These pre-miRNAs typically undergo an additional processing step within the cytoplasm where mature miRNAs of 18-25 nucleotides in length are excised from one side of the pre-miRNA hairpin by an RNase III enzyme, Dicer. miRNAs are not translated into proteins, but instead bind to specific messenger RNAs, thereby blocking translation. In some embodiments, miRNAs base-pair imprecisely with their targets to inhibit translation.

miRNAs as described herein can include pri-miRNA, pre-miRNA, mature miRNA or fragments of variants thereof that retain the biological activity of mature miRNA. In some embodiments, the size range of the miRNA can be from 21 nucleotides to 170 nucleotides. In one embodiment, the size range of the miRNA is from 70 to 170 nucleotides in length. In another embodiment, mature miRNAs of from 21 to 25 nucleotides in length can be used.

In certain embodiments, the interfering nucleic acid molecule is an antisense oligonucleotide (ASO). An ASO can down regulate a target by inducing RNase H endonuclease cleavage of a target RNA, by steric hindrance of ribosomal activity, by inhibiting 5′ cap formation, or by altering splicing. An ASO can be, but is not limited to, a gapmer or a morpholino. An antisense oligonucleotide typically comprises a short nucleotide sequence which is substantially complementary to a target nucleotide sequence in a pre-mRNA molecule, heterogeneous nuclear RNA (hnRNA) or mRNA molecule. The degree of complementarity (or substantial complementarity) of the antisense sequence is preferably such that a molecule comprising the antisense sequence can form a stable double stranded hybrid with the target nucleotide sequence in the RNA molecule under physiological conditions. Antisense oligonucleotides are often synthetic and chemically modified.

Antisense oligonucleotides may be 100% complementary to the target sequence, or may include mismatches, e.g., to improve selective targeting of allele containing the disease-associated mutation, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability.

In some embodiments, an interfering nucleic acid molecule described herein is a gapmer. A “Gapmer” is oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” A gapmer can have 5′ and 3′ wings each having 2-6 nucleotides and a gap having 7-12 nucleotides. In some embodiments, a gapmer can have a 3-10-3 configuration or a 5-10-5 configuration.

A gapmer commonly has the formula 5′-X—Y—Z-3′, with X and Z as flanking regions around a gap region Y. In some embodiments, flanking region X of formula 5′-X—Y—Z-3′ is also called X region, flanking sequence X, 5′ wing region X, or 5′ wing segment. In some embodiments, flanking region Z of formula 5′-X—Y—Z-3′ is also called Z region, flanking sequence Z, 3′ wing region Z, or 3′ wing segment. In some embodiments, gap region Y of formula 5′-X—Y—Z-3′ is also called Y region, Y segment, gap-segment Y, gap segment, or gap region. In some embodiments, each nucleoside in the gap region Y is a 2′-deoxyribonucleoside, and neither the 5′ wing region X or the 3′ wing region Z comprises any 2′-deoxyribonucleosides.

In some embodiments, the gap region of the gapmer polynucleotide may contain modified nucleotides known to be acceptable for efficient RNase H action in addition to DNA nucleotides, such as C4′-substituted nucleotides, acyclic nucleotides, and arabino-configured nucleotides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, or at least five or more nucleotides. In some embodiments, each internucleotide linkage in the gap segment comprises a phosphorothioate linkage. In some embodiments, the gap region and two flanking regions each independently comprise modified internucleoside linkages (e.g., phosphorothioate internucleoside linkages or other linkages) between at least two, at least three, at least four, or at least five or more nucleotides. In some embodiments, each internucleotide linkage in the 5′ or 3′ wing region comprises a phosphorothioate linkage. In some embodiments, each internucleotide linkage in the gapmer comprises a phosphorothioate linkage.

In some embodiments, the Y region may comprise a contiguous stretch of nucleotides, e.g., a region of 5 or more DNA nucleotides, which can be capable of recruiting an RNase including but not limited to Rnase H. In some embodiments, the gapmer may bind to a target nucleic acid such that an Rnase is recruited to cleave the target nucleic acid. In some embodiments, the Y region may be flanked both 5′ and 3′ by regions X and Z comprising high-affinity modified nucleosides, e.g., 1-10 high-affinity modified nucleosides. Exemplary high affinity modified nucleosides include, without limitation, 2′-4′ bicyclic nucleosides (e.g., LNA, cEt, ENA) and 2′-modified nucleosides (e.g., 2′-MOE, 2′O-Me, 2′-F). In some embodiments, the flanking sequences X and Z may be of 1-30 nucleotides, 1-20 nucleotides, 1-10 nucleotides, or 1-5 nucleotides in length. The flanking sequences X and Z may be of similar length or of dissimilar lengths. In some embodiments, the flanking sequences X and Z are each 5 nucleotides in length. In some embodiments, the flanking sequences X and Z are each 3 nucleotides in length. In some embodiments, the gap-segment Y may be a nucleotide sequence of 5-30 nucleotides, 5-20 nucleotides, or 5-10 nucleotides in length. In some embodiments, the gap segment is 10 nucleotides in length.

A gapmer may be produced using suitable methods. Preparation of gapmers is described in, for example, U.S. Pat. Nos. 10,260,069; 10,017,764; 9,695,418; 9,428,534; 9,428,534; 9,045,754; 8,580,756; 8,580,756; 7,750,131; 7,683,036; 7,569,686; 7,432,250; 7,399,845; 7,101,993; 7,015,315; 5,898,031; 5,700,922; 5,652,356; 5,652,355; 5,623,065; 5,565,350; 5,491,133; 5,403,711; 5,366,878; 5,256,775; 5,220,007; 5,149,797; and 5,013,830; U.S. Patent Publication Nos. US2010/0197762, US2005/0074801, US2009/0221685, US2009/0286969, and US2011/0112170; PCT Publication Nos. WO2005/023825, WO2004/069991, WO2008/049085 and WO2009/090182, each of which is herein incorporated by reference in its entirety.

In some embodiments, a gapmer is 10-50 nucleosides in length. For example, a gapmer may be 10-50, 10-45, 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35, or 35-40 nucleosides in length. In some embodiments, a gapmer is 10, 11, 12, 13, 14, 15, 16, 17, 18, 19,20, 21, 22, 23, 24, 25, 26, 27, 28, 29, 30, 31, 32, 33, 34, 35, 36, 37, 38, 39, or 40 nucleosides in length. In some embodiments, a gapmer is about 16 to about 20 nucleosides in length. In some embodiments, a gapmer is 16 nucleotides in length. In some embodiments, a gapmer is 20 nucleotides in length.

In some embodiments, the 5′ wing region and the 3′ wing region of a gapmer are independently 1-20 nucleosides (e.g., 1, 2, 3, 4, 5, 6, 7, 8, 9, 10, 11, 12, 13, 14, 15, 16, 17, 18, 19, or 20 nucleosides) long. For example, the 5′ wing region and the 3′ wing region of the gapmer may be independently 1-20, 1-15, 1-10, 1-7, 1-5, 1-3, 1-2, 2-5, 2-7, 3-5, 3-7, 5-20, 5-15, 5-10, 10-20, 10-15, or 15-20 nucleosides long. In some embodiments, the 5′ wing region and the 3′ wing region of the gapmer are of the same length. In some embodiments, the 5′ wing region and the 3′ wing region of a gapmer are of different lengths. In some embodiments, the 5′ wing region is longer than the 3′ wing region of a gapmer. In some embodiments, the 5′ wing region is shorter than the 3′ wing region of the gapmer.

In some embodiments, the gap region in a gapmer is 5-20 nucleosides in length. For example, the gap region Y may be 5-20, 5-15, 5-10, 10-20, 10-15, or 15-20 nucleosides in length. In some embodiments, the gap region is 5, 6, 7, 8, 9, 10, 11, 12, 13, 14, 15, 16, 17, 18, 19, or 20 nucleosides in length. In some embodiments, one or more nucleosides in the gap region Y is a 2′-deoxyribonucleoside. In some embodiments, every nucleotide in the gap region is a deoxyribonucleoside. In some embodiments, one or more of the nucleosides in the gap region is a modified nucleoside (e.g., a 2′ modified nucleoside such as those described herein). In some embodiments, one or more cytosines in the gap region Y are 5-methyl-cytosines. In some embodiments, every cytosine in the gap region Y is a 5-methyl-cytosine. In some embodiments, every cytosine in a gapmer is a 5-methyl-cytosine.

In some embodiments, one or more nucleosides in the 5 wing region or the 3′ wing region of a gapmer are modified nucleotides. In some embodiments, the modified nucleotide may be a 2′-modified nucleoside, e.g., 2′-4′ bicyclic nucleoside or a non-bicyclic 2′-modified nucleoside. In some embodiments, the nucleoside may be a 2′-4′ bicyclic nucleoside (e.g., LNA, cEt, or ENA) or a non-bicyclic 2′-modified nucleoside (e.g., 2′-fluoro (2′-F), 2′-O-methyl (2′-O-Me), 2′-O-dimethylaminoethyl (2′-O-DMAOE), 2′-O-dimethylaminopropyl (2′-O-DMAP), 2′-O-methoxyethyl (2′-MOE), 2′-O-aminopropyl (2′-O-AP), 2′-O-dimethylaminoethyloxyethyl (2′-O-DMAEOE), or 2′-O—N-methylacetamido (2′-O-NMA)). In some embodiments, every nucleotide in a wing region is a modified nucleotide. In some embodiments, every nucleotide in a wing region is a 2′-MOE, LNA or cET nucleotide.

In some embodiments, a gapmer described herein may comprises one or more modified nucleoside linkages in each of the X, Y, and Z regions. In some embodiments, each internucleoside linkage may comprise phosphorothioate linkage. In some embodiments, each of the X, Y, and Z regions independently comprises a combination of phosphodiester linkages and phosphorothioate linkages. In some embodiments, each internucleoside linkage in the gap region Y may be a phosphorothioate linkage, the 5′ wing region X comprises a combination of phosphorothioate linkages and phosphodiester linkages, and the 3′ wing region Z comprises a combination of phosphorothioate linkages and phosphodiester linkages.

In some embodiments, each nucleotide in the gap region of a gapmer is a deoxyribonucleotide and each nucleotide in a wing region is a 2′-MOE nucleotide. In some embodiments, each nucleotide in the gap region of a gapmer is a deoxyribonucleotide, each nucleotide in a wing region is a 2′-MOE nucleotide, and every cytosine in the gapmer is a 5-methyl-cytosine. In some embodiments, each nucleotide in the gap region of a gapmer is a deoxyribonucleotide, each nucleotide in a wing region is a 2′-MOE nucleotide, every cytosine in the gapmer is a 5-methyl-cytosine and every internucleotide linkage is a phosphorothioate linkage.

In some embodiments, each nucleotide in the gap region of a gapmer is a deoxyribonucleotide and each nucleotide in a wing region is a LNA nucleotide. In some embodiments, each nucleotide in the gap region of a gapmer is a deoxyribonucleotide, each nucleotide in a wing region is a LNA nucleotide, and every cytosine in the gapmer is a 5-methyl-cytosine. In some embodiments, each nucleotide in the gap region of a gapmer is a deoxyribonucleotide, each nucleotide in a wing region is a LNA nucleotide, every cytosine in the gapmer is a 5-methyl-cytosine and every internucleotide linkage is a phosphorothioate linkage. In some embodiments, each nucleotide in the gap region of a gapmer is a deoxyribonucleotide and each nucleotide in a wing region is a cET nucleotide. In some embodiments, each nucleotide in the gap region of a gapmer is a deoxyribonucleotide, each nucleotide in a wing region is a cET nucleotide, and every cytosine in the gapmer is a 5-methyl-cytosine. In some embodiments, each nucleotide in the gap region of a gapmer is a deoxyribonucleotide, each nucleotide in a wing region is a cET nucleotide, every cytosine in the gapmer is a 5-methyl-cytosine and every internucleotide linkage is a phosphorothioate linkage.

The interfering nucleic acids can employ a variety of oligonucleotide chemistries. Examples of oligonucleotide chemistries include, without limitation, peptide nucleic acid (PNA), locked nucleic acid (LNA), phosphorothioate, 2′-O-Me-modified oligonucleotides, and morpholino chemistries, including combinations of any of the foregoing. In general, PNA and LNA chemistries can utilize shorter targeting sequences because of their relatively high target binding strength relative to 2′-O-Me oligonucleotides. Phosphorothioate and 2′-O-Me-modified chemistries are often combined to generate 2′-O-Me-modified oligonucleotides having a phosphorothioate backbone. See, e.g., PCT Publication Nos. WO/2013/112053 and WO/2009/008725, incorporated by reference in their entireties.

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications. The backbone is uncharged, resulting in PNA/DNA or PNA/RNA duplexes that exhibit greater than normal thermal stability. PNAs are not recognized by nucleases or proteases.

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™ has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 5,539,082; 5,714,331; and 5,719,262, 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

Interfering nucleic acids described herein may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230. Compounds provided herein may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties. Alternatively, non-phosphorous containing linkers may be employed. In some embodiments, an antisense oligonucleotides comprises an LNA containing compound where each LNA subunit is separated by a DNA subunit. Certain compounds are composed of alternating LNA and DNA subunits where the intersubunit linker is phosphorothioate.

“Phosphorothioates” (or S-oligos) are a variant of normal DNA in which one of the nonbridging oxygens is replaced by a sulfur. The sulfurization of the internucleotide bond reduces the action of endo- and exonucleases including 5′ to 3′ and 3′ to 5′ DNA POL 1 exonuclease, nucleases SI and PI, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1,2-bensodithiol-3-one 1,1-dioxide (BDTD) (see, e.g., Iyer et al., J. Org. Chem. 55, 4693-4699, 1990). The latter methods avoid the problem of elemental sulfur's insolubility in most organic solvents and the toxicity of carbon disulfide. The TETD and BDTD methods also yield higher purity phosphorothioates.

“2′ O-Me oligonucleotides” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphothioate oligonucleotides (PTOs) for further stabilization. 2′-O-Me oligonucleotides (phosphodiester or phosphothioate) can be synthesized according to routine techniques in the art (see, e.g., Yoo et al., Nucleic Acids Res. 32:2008-16, 2004).

Interfering nucleic acid molecules can be prepared, for example, by chemical synthesis, in vitro transcription, or digestion of long dsRNA by RNase III or Dicer. These can be introduced into cells by transfection, electroporation, or other methods known in the art. See Hannon, G J, 2002, Nature 418: 244-251; Bernstein E et al., 2002, RNA 7: 1509-1521; Hutvagner G et al., Curr. Opin. Genetics & Development 12: 225-232; Brummelkamp, 2002, Science 296: 550-553; Lee N S, et al. 2002. Nature Biotechnol. 20:500-505; Miyagishi M, and Taira K. 2002. Nature Biotechnol. 20:497-500; Paddison P J, et al., 2002. Genes & Dev. 16:948-958; Paul C P, et al., 2002. Nature Biotechnol. 20:505-508; Sui G et al., 2002. Proc. Natl. Acad. Sci. USA 99(6):5515-5520; Yu J-Y et al., 2002. Proc. Natl. Acad. Sci. USA 99(9):6047-6052. Each of the foregoing is incorporated by reference in its entirety.

### Guide RNAs

In some embodiments, a conjugated molecular cargo comprises a guide RNA or a DNA encoding a guide RNA. A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and Cas Φ, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUUUUAGAGCUAUGCU (SEQ ID NO: 321) or GUUUUAGAGCUAUGCUGUUUUG (SEQ ID NO: 322). Any of the DNA-targeting segments disclosed herein can be joined to the 5′ end of SEQ ID NO: 321 or 322 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences (e.g., for use with S. pyogenes Cas9) comprise, consist essentially of, or consist of any one of AGCAUAGCAAGUUAAAAUAAGGCUAGUCCGUUAUCAACUUGAAAAAGUGGCACCG AGUCGGUGCUUU (SEQ ID NO: 323), AAACAGCAUAGCAAGUUAAAAUAAGGCUAGUCCGUUAUCAACUUGAAAAAGUGGC ACCGAGUCGGUGCUUUU (SEQ ID NO: 324), or GUUGGAACCAUUCAAAACAGCAUAGCAAGUUAAAAUAAGGCUAGUCCGUUAUCAA CUUGAAAAAGUGGCACCGAGUCGGUGC (SEQ ID NO: 325).

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, or 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of: GUUUUAGAGCUAGAAAUAGCAAGUUAAAAUAAGGCUAGUCCGUUAUCAACUUGAA AAAGUGGCACCGAGUCGGUGCU (version 1; SEQ ID NO: 427); GUUGGAACCAUUCAAAACAGCAUAGCAAGUUAAAAUAAGGCUAGUCCGUUAUCAA CUUGAAAAAGUGGCACCGAGUCGGUGC (version 2; SEQ ID NO: 325); GUUUUAGAGCUAGAAAUAGCAAGUUAAAAUAAGGCUAGUCCGUUAUCAACUUGAA AAAGUGGCACCGAGUCGGUGC (version 3; SEQ ID NO: 429); and GUUUAAGAGCUAUGCUGGAAACAGCAUAGCAAGUUUAAAUAAGGCUAGUCCGUU AUCAACUUGAAAAAGUGGCACCGAGUCGGUGC (version 4; SEQ ID NO: 430); GUUUUAGAGCUAGAAAUAGCAAGUUAAAAUAAGGCUAGUCCGUUAUCAACUUGAA AAAGUGGCACCGAGUCGGUGCUUUUUUU (version 5; SEQ ID NO: 431); GUUUUAGAGCUAGAAAUAGCAAGUUAAAAUAAGGCUAGUCCGUUAUCAACUUGAA AAAGUGGCACCGAGUCGGUGCUUUU (version 6; SEQ ID NO: 432); GUUUAAGAGCUAUGCUGGAAACAGCAUAGCAAGUUUAAAUAAGGCUAGUCCGUU AUCAACUUGAAAAAGUGGCACCGAGUCGGUGCUUUUUU (version 7; SEQ ID NO: 433); or GUUUUAGAGCUAGAAAUAGCAAGUUAAAAUAAGGCUAGUCCGUUAUCAACUUGG CACCGAGUCGGUGC (version 8; SEQ ID NO: 434). In some guide sgRNAs, the four terminal U residues of version 6 are not present. In some sgRNAs, only 1, 2, or 3 of the four terminal U residues of version 6 are present. Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

In some cases, a guide RNA for use in a transcriptional activation system comprising a dCas9-VP64 fusion protein paired with MS2-p65-HSF1 can be used. Guide RNAs in such systems can be designed with aptamer sequences appended to sgRNA tetraloop and stem-loop 2 designed to bind dimerized MS2 bacteriophage coat proteins. See, e.g., Konermann et al. (2015) Nature 517(7536):583-588, herein incorporated by reference in its entirety for all purposes.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such at hose listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively or additionally, a modified gRNA can comprise at least one modified residue at or near the 5′ end. Alternatively or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

The gRNAs disclosed herein can comprise a backbone modification in which the phosphate group of a modified residue can be modified by replacing one or more of the oxygens with a different substituent. The modification can include the wholesale replacement of an unmodified phosphate moiety with a modified phosphate group as described herein. Backbone modifications of the phosphate backbone can also include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (Rp) or the “S” configuration (Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group (a sugar modification). For example, the 2′ hydroxyl group (OH) can be modified (e.g., replaced with a number of different oxy or deoxy substituents. Modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). The 2′ hydroxyl group modification can be 2′-O-Me. Likewise, the 2′ hydroxyl group modification can be a 2′-fluoro modification, which replaces the 2′ hydroxyl group with a fluoride. The 2′ hydroxyl group modification can include locked nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). The 2′ hydroxyl group modification can include unlocked nucleic acids (UNA) in which the ribose ring lacks the C2′—C3′ bond. The 2′ hydroxyl group modification can include the methoxyethyl group (MOE), (OCH₂CH₂OCH₃, e.g., a PEG derivative).

Deoxy 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH₂CH₂NH)ₙCH₂CH₂—amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form (e.g. L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22(9):2227-2235, each of which is herein incorporated by reference in its entirety for all purposes. Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively or additionally, the at least one modification can comprise a phosphorothioate (PS) bond between nucleotides. Alternatively or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

In one example, a modified gRNA can comprise the following sequence: mN*mN*mN*NNNNNNNNNNNNNNNNNGUUUUAGAmGmCmUmAmGmAmAmAmUm AmGmCAAGUUAAAAUAAGGCUAGUCCGUUAUCAmAmCmUmUmGmAmAmAmAmA mGmUmGmGmCmAmCmCmGmAmGmUmCmGmGmUmGmCmU*mU*mU*mU (SEQ ID NO: 435), where “N” may be any natural or non-natural nucleotide. The totality of N residues can comprise a DNA-targeting segment as described herein. The terms “mA,” “mC,” “mU,” and “mG” denote a nucleotide (A, C, U, and G, respectively) that has been modified with 2′-O-Me. The symbol “*” depicts a phosphorothioate modification. In certain embodiments, A, C, G, U, and N independently denote a ribose sugar, i.e., 2′-OH. In certain embodiments in the context of a modified sequence, A, C, G, U, and N denote a ribose sugar, i.e., 2′-OH. A phosphorothioate linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos. The terms A*, C*, U*, or G* denote a nucleotide that is linked to the next (e.g., 3′) nucleotide with a phosphorothioate bond. The terms “mA*,” “mC*,” “mU*,” and “mG*” denote a nucleotide (A, C, U, and G, respectively) that has been substituted with 2′-O-Me and that is linked to the next (e.g., 3′) nucleotide with a phosphorothioate bond.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Abasic nucleotides refer to those which lack nitrogenous bases. Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In one example, one or more of the first three, four, or five nucleotides at the 5′ terminus, and one or more of the last three, four, or five nucleotides at the 3′ terminus are modified. The modification can be, for example, a 2′-O-Me, 2′-F, inverted abasic nucleotide, phosphorothioate bond, or other nucleotide modification well known to increase stability and/or performance.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

Guide RNAs can be provided in any form. For example, the gRNA can be conjugated to the anti-TfR antigen-binding protein disclosed herein, such as an scFv or an antibody or an antigen-binding fragment thereof, in the form of RNA, either as two molecules (separate crRNA and tracrRNA) or as one molecule (sgRNA), and optionally in the form of a complex with a Cas protein.

The gRNA can be conjugated to the anti-TfR antigen-binding protein disclosed herein, such as an scFv or an antibody or an antigen-binding fragment thereof, in the form of DNA encoding the gRNA. The DNA encoding the gRNA can encode a single RNA molecule (sgRNA) or separate RNA molecules (e.g, separate crRNA and tracrRNA). In the latter case, the DNA encoding the gRNA can be provided as one DNA molecule or as separate DNA molecules encoding the crRNA and tracrRNA, respectively.

Multiple gRNAs can be conjugated to the anti-TfR antigen-binding protein disclosed herein, such as an scFv or an antibody or an antigen-binding fragment thereof. The gRNAs can be the same or different gRNAs, or can target the same gene or different genes. In some embodiments, 1, 2, 3, 4, 5 or more guide RNAs are conjugated to the anti-TfR antigen-binding protein disclosed herein, such as an scFv or an antibody or an antigen-binding fragment thereof.

Alternatively, the gRNA, either in the form of RNA or DNA, may be incorporated into a carrier (e.g., liposomes or LNPs) which is conjugated to the anti-TfR antigen-binding protein disclosed herein, such as an scFv or an antibody or an antigen-binding fragment thereof. The carrier can further comprise a Cas protein, such as a Cas9 protein, or a nucleic acid (e.g., mRNA) encoding a Cas protein. Carriers such as liposomes or lipid nanoparticles are described in further detail below.

Multiple gRNAs can be incorporated into a carrier (e.g., liposome or LNP) which is conjugated to the anti-TfR antigen-binding protein disclosed herein, such as an scFv or an antibody or an antigen-binding fragment thereof. The gRNAs can be the same or different gRNAs, or can target the same gene or different genes. In some embodiments, 1, 2, 3, 4, 5 or more guide RNAs are incorporated into a carrier (e.g., liposome or LNP) which is conjugated to the anti-TfR antigen-binding protein disclosed herein, such as an scFv or an antibody or an antigen-binding fragment thereof.

When a gRNA is provided in the form of DNA, the gRNA after being delivered to the target cell can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand”.

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

The target sequence (e.g., guide RNA target sequence) for the DNA-binding protein can be anywhere within a targeted gene that is suitable for altering expression of the targeted gene. As one example, the target sequence can be within a regulatory element, such as an enhancer or promoter, or can be in proximity to a regulatory element. For example, the target sequence can be within about 10, 20, 30, 40, 50, 100, 200, 300, 400, 500, or 1,000 nucleotides of the start codon.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g, 3 base pairs) upstream or downstream of the PAM sequence (e.g., within the guide RNA target sequence). In the case of SpCas9, the PAM sequence (i.e., on the non-complementary strand) can be 5′-NiGG-3′, where Ni is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN2-3′, where N2 is any DNA nucleotide and is immediately 5′ of the sequence to which the DNA-targeting segment of the guide RNA hybridizes on the complementary strand of the target DNA. In some such cases, Ni and N2 can be complementary and the Ni—N2 base pair can be any base pair (e.g., Ni=C and N2=G; Ni=G and N2=C; Ni=A and N2=T; or Ni=T, and N2=A). In the case of Cas9 from S. aureus, the PAM can be NNGRRT (SEQ ID NO: 503) or NNGRR (SEQ ID NO: 504), where N can A, G, C, or T, and R can be G or A. In the case of Cas9 from C. jejuni, the PAM can be, for example, NNNNACAC (SEQ ID NO: 505) or NNNNRYAC (SEQ ID NO: 506), where N can be A, G, C, or T, and R can be G or A. In some cases (e.g., for FnCpf1), the PAM sequence can be upstream of the 5′ end and have the sequence 5′-TTN-3. In the case of DpbCasX, the PAM can have the sequence 5′-TTCN-3′. In the case of CasΦ, the PAM can have the sequence 5′-TBN-3′, wherein B is G, T, or C.

An example of a guide RNA target sequence is a 20-nucleotide DNA sequence immediately preceding an NGG motif recognized by an SpCas9 protein. The guanine at the 5′ end can facilitate transcription by RNA polymerase in cells. Other examples of guide RNA target sequences plus PAMs can include two guanine nucleotides at the 5′ end to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length, including the 5′ G or GG and the 3′ GG or NGG. Yet other guide RNA target sequences plus PAMs can have between 14 and 20 nucleotides in length.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g., Cas9) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

### Other Types of Polynucleotide Molecules

In some embodiments, a molecular cargo, e.g., a polynucleotide molecule described herein may comprise a ribozyme (ribonucleic acid enzyme). Without wishing to be bound by theory, a ribozyme is a molecule, commonly an RNA molecule, that is capable of performing specific biochemical reactions, akin to the action of protein enzymes. Ribozymes comprise molecules possessing catalytic activities such as, but not limited to, the capacity to cleave at specific phosphodiester linkages in RNA molecules to which they have hybridized, e.g., RNA-containing substrates, lncRNAs, mRNAs, and ribozymes.

Ribozymes may take on one of several physical structures, one such structure is termed “hammerhead”. A hammerhead ribozyme can comprise, e.g., a catalytic core comprising nine conserved bases, two regions complementary to the target RNA flanking regions the catalytic core, and a double-stranded stem and loop structure (stem-loop II). The flanking regions may permit the binding of the ribozyme to the target RNA, in particular, by forming double-stranded stems I and III. Cleavage may occur in trans (cleavage of an RNA substrate other than that containing the ribozyme) or in cis (cleavage of the same RNA molecule that contains the hammerhead motif) adjacent to a specific ribonucleotide triplet by a transesterification reaction from a 3′,5′-phosphate diester to a 2′,3′-cyclic phosphate diester. In certain embodiments, this catalytic activity may require the presence of specific, highly conserved sequences in the catalytic region of the ribozyme.

Modifications in ribozyme structure can include the replacement or substitution of non-core portions of the molecule with non-nucleotidic molecules. As a non-limiting example, Ma et al. (Biochem. (1993) 32:1751-1758; Nucleic Acids Res. (1993) 21:2585-2589) replaced the six-nucleotide loop of the TAR ribozyme hairpin with non-nucleotidic, ethylene glycol-related linkers. Thomson et al. (Nucleic Acids Res. (1993) 21:5600-5603) replaced loop II with linear, non-nucleotidic linkers of 13, 17, and 19 atoms in length. Benseler et al. (J. Am. Chem. Soc. (1993) 115:8483-8484) describes hammerhead-like molecules where two of the base pairs of stem II, and all four of the nucleotides of loop II may be replaced with non-nucleoside linkers based on bis(propanediol) phosphate, hexaethylene glycol, bis(triethylene glycol) phosphate, propanediol, or tris(propanediol)bisphosphate.

Ribozyme polynucleotides may be generated using any of various suitable methods known in the art (see, e.g., U.S. Pat. Nos. 5,436,143 and 5,650,502; and PCT Publications Nos. WO94/13688; WO91/18624, WO92/01806; and WO 92/07065) or can be obtained from commercial sources (e.g., US Biochemicals), the contents of each of which are incorporated herein by reference in their entirety. In some embodiments, the ribozyme polynucleotide described herein can incorporate nucleotide analogs, e.g., to increase the resistance of the oligonucleotide to degradation by nucleases in a cell. The ribozyme may be synthesized in any known manner, e.g., by use of a commercially available synthesizer produced, e.g., by Applied Biosystems, Inc. or Milligen. The ribozyme RNA sequences maybe synthesized conventionally, for example, by using RNA polymerases such as T7 or SP6. The ribozyme may also be produced in recombinant vectors by suitable means.

In some embodiments, internucleotidic phosphorus atoms of the polynucleotide molecules disclosed herein may be chiral, and the properties of the polynucleotides by adjusted based on the configuration of the chiral phosphorus atoms.

In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43, the contents of which are incorporated herein by reference in their entirety). In some embodiments, phosphorothioate-containing oligonucleotides may comprise nucleoside units that can be joined together by either substantially all Rp or substantially all Sp phosphorothioate inter-sugar linkages. In some embodiments, such phosphorothioate oligonucleotides comprising substantially chirally pure inter-sugar linkages may be produced via chemical synthesis or enzymatic approaches, as disclosed, e.g., in U.S. Pat. No. 5,587,261, the contents of which are incorporated herein by reference in their entirety. In some embodiments, chirally controlled polynucleotide molecules described may provide selective cleavage patterns of a target nucleic acid. As a non-limiting example, a chirally controlled polynucleotide molecule may provide single site cleavage within a complementary sequence of a nucleic acid, as disclosed, for example, in US Patent Publication No. 2017/0037399, the contents of which are incorporated herein by reference in their entirety.

In some embodiments, the polynucleotide molecule described herein may be a morpholino-based compound. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties). Morpholino-based oligomeric compounds are also described in, e.g., U.S. Pat. No. 5,034,506, and Genesis, volume 30, issue 3, 2001; Heasman, J., Dev. Biol., 2002, 243, 209-214; Dwaine A. Braasch and David R. Corey, Biochemistry, 2002, 41(14), 4503-4510); Nasevicius et al., Nat. Genet., 2000, 26, 216-220; Lacerra et al., Proc. Natl. Acad. Sci., 2000, 97, 9591-9596, the disclosures of which are incorporated herein by reference in their entireties.

In some embodiments, a polynucleotide molecule described herein may comprise an aptamer. An aptamer may comprise any nucleic acid which specifically binds specifically to a target, e.g., protein or nucleic acid in a cell. In some embodiments, the aptamer is a DNA aptamer or an RNA aptamer. In some embodiments, a nucleic acid aptamer may comprise a single-stranded RNA (ssDNA or ssRNA) or DNA. In certain embodiments, a single-stranded nucleic acid aptamer may form loop(s) and/or helice(s) structures. The nucleic acid that forms the nucleic acid aptamer may comprise naturally occurring nucleotides, modified nucleotides with hydrocarbon or PEG linkers inserted between one or more nucleotides, modified nucleotides, naturally occurring nucleotides with hydrocarbon linkers (e.g., an alkylene) or a polyether linker (e.g., a PEG linker) inserted between one or more nucleotides, or a combination of thereof. Aptamers and method of producing aptamers are described in, e.g., U.S. Pat. Nos. 8,318,438, 5,650,275; 5,683,867; 5,670,637; 5,696,249; 5,789,157; 5,843,653; 5,270,163; 5,567,588, 5,864,026; 5,989,823; 6,569,630; and PCT Publication No. WO 99/31275, Lorsch and Szostak, 1996; Jayasena, 1999; each incorporated herein by reference.

In some embodiments, a polynucleotide molecule described herein may be a mixmer or comprise a mixmer sequence pattern. In some embodiments, mixmers can be polynucleotides that comprise both naturally and non-naturally occurring nucleosides or comprise two different types of non-naturally occurring nucleosides commonly in an alternating pattern. Mixmers may have higher binding affinity than unmodified polynucleotides and may be used, in particular, to specifically bind a target molecule, e.g., to block a binding site on the target molecule. In some embodiments, mixmers may not recruit an RNase to a target molecule and hence do not promote cleavage of the target molecule. Such polynucleotides that may be incapable of recruiting, e.g., RNase H have been described, e.g., see WO2007/112753 or WO2007/112754.

In some embodiments, a mixmer disclosed herein may comprise a repeating pattern of naturally occurring nucleosides and nucleoside analogues, or, e.g., one type of nucleoside analogue and a second type of nucleoside analogue. Yet, a mixmer need not comprise a repeating pattern and may instead comprise any arrangement of modified naturally occurring nucleosides and nucleosides or any arrangement of one type of modified nucleoside and a second type of modified nucleoside. Such repeating pattern, may, for example comprise every second or every third nucleoside as a modified nucleoside, e.g., LNA. In certain embodiments, the remaining nucleosides may be naturally occurring nucleosides, e.g., DNA, or may be a 2′ substituted nucleoside analogue, e.g., 2′ fluoro analogues or 2′-MOE, or any other some modified nucleoside(s) disclosed herein. It is understood that the repeating pattern of modified nucleoside, such as LNA units, may be combined with modified nucleoside at fixed positions (e.g., at the 5′ and/or 3′ termini).

In some embodiments, a mixmer may not comprise a region of more than 6. more than 5, more than 4, more than 3, or more than 2 consecutive naturally occurring nucleosides (e.g., DNA nucleosides). In some embodiments, the mixmer may comprise at least a region comprising at least two consecutive modified nucleosides, for example, at least two consecutive LNAs. In some embodiments, the mixmer may comprise at least a region consisting of at least three consecutive modified nucleoside units, e.g., at least three consecutive LNAs.

In some embodiments, the mixmer may not comprise a region of more than 8, more than 7, more than 6, more than 5, more than 4, more than 3, or more than 2 consecutive nucleoside analogues, e.g., LNAs. In some embodiments, LNA units may be replaced with other nucleoside analogues including, but not limited to, those referred to herein.

In some embodiments, mixmers may be designed to comprise a mixture of affinity enhancing modified nucleosides, such as, without limitation, in LNA nucleosides and 2′-O-Me nucleosides. In some embodiments, a mixmer may comprise modified internucleoside linkages (e.g., phosphorothioate internucleoside linkages or other linkages) between at least two, at least three, at least four, at least five, at least six or more nucleosides.

In some embodiments, a mixmer may comprise one or more morpholino nucleosides. In some embodiments, a mixmer may comprise morpholino nucleosides mixed (e.g., in an alternating manner) with one or more other nucleosides (e.g., DNA, RNA nucleosides) or modified nucleosides (e.g., 2′-O-Me nucleosides, LNA).

In some embodiments, mixmers may be useful for splice correcting or exon skipping, for example, as described in Chen S. et al., Molecules 2016, 21, 1582, Touznik A., et al., Scientific Reports, volume 7, Article number: 3672 (2017), the contents of each which are incorporated herein by reference.

A mixmer may be produced using any suitable method. Preparation of mixmers is described in, for example, U.S. Pat. No. 7,687,617, and U.S. Patent Application Publication Nos. US2012/0322851, US2009/0209748, US2009/0298916, US2006/0128646, and US2011/0077288. Additional examples of multimers are described, for example, in U.S. Pat. No. 5,693,773, US Patent Application Publication Nos. 2015/0247141; 2015/0315588; US 2011/0158937; the contents of each of which are incorporated herein by reference in their entireties.

In some embodiments, polynucleotide molecules comprising molecular cargos disclosed herein may comprise multimers (e.g., concatemers) of two or more polynucleotide molecules connected, e.g., by a linker. Polynucleotides in a multimer may be the same or different (e.g., targeting different sites on the same gene different genes or products thereof).

In some embodiments, multimers may comprise two or more polynucleotide molecules linked together by a cleavable linker. In some embodiments, multimers may comprise two or more polynucleotide molecules linked together, e.g., by a non-cleavable linker. In some embodiments, a multimer may comprise 2, 3, 4, 5, 6, 7, 8, 9, 10, 11, 12, 13, 14, 15, 16, 17, 18, 19, 20 or more polynucleotide molecules linked together. In some embodiments, a multimer may comprises 2 to 5, 2 to 10, 4 to 20 or 5 to 30 polynucleotide molecules linked together.

In some embodiments, a multimer may comprises two or more polynucleotide molecules linked in a linear arrangement, e.g., end-to-end. In some embodiments, a multimer may comprises two or more polynucleotide molecules linked end-to-end via a polynucleotide-based linker (e.g., an abasic linker, a poly-dT linker). In some embodiments, a multimer comprises a 3′ end of one polynucleotide linked to a 3′ end of another polynucleotide. In some embodiments, a multimer may comprise a 5′ end of one polynucleotide linked to a 3′ end of another polynucleotide. In some embodiments, a multimer comprises a 5′ end of one polynucleotide linked to a 5′ end of another polynucleotide. In some embodiments, multimers may comprise a branched structure comprising multiple polynucleotides linked together by a branching linker.

In some embodiments, a polynucleotide molecule of the present disclosure can target splicing. In some embodiments, the polynucleotide can targets splicing by inducing exon skipping and restoring the reading frame within a gene. For example, without limitation, the oligonucleotide may induce skipping of an exon encoding a frameshift mutation and/or an exon that encodes a premature stop codon. In some embodiments, a polynucleotide may induce exon skipping by, e.g., blocking spliceosome recognition of a splice site. In some embodiments, a polynucleotide molecule disclosed herein may induce inclusion of an exon by targeting a splice site inhibitory sequence. In some embodiments, the oligonucleotide promotes inclusion of a particular exon. In some embodiments, exon skipping results in a truncated but functional protein compared to the reference protein.

In some embodiments, the polynucleotide molecule described herein may be a messenger RNA (mRNA). mRNAs comprise an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof. Bases of an mRNA can be modified bases such as pseudouridine, N-1-methyl-pseudouridine, or other naturally occurring or non-naturally occurring bases.

### Liposomes, Lipid Nanoparticles and Other Carriers

In some embodiments, a conjugated molecular cargo described herein comprises a carrier, for example, a lipid-based carrier, such as a lipid nanoparticle (LNP), a liposome, a lipidoid, or a lipoplex, a polymeric nanoparticle, an inorganic nanoparticle, a peptide carrier, a nanoparticle mimic, or a nanotube.

In some embodiments, a conjugated molecular cargo described herein comprises a liposome or LNP. Liposomes and LNPs are vesicles including one or more lipid bilayers. In some embodiments, a liposome or LNP includes two or more concentric bilayers separated by aqueous compartments. Lipid bilayers can be functionalized and/or crosslinked to one another. Lipid bilayers can include one or more proteins, polysaccharides or other molecules.

Lipid formulations can protect biological molecules from degradation while improving their cellular uptake. Liposomes or LNPs are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such liposomes or LNPs can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as distearoylphosphatidylcholine (DSPC). In another example, the other components can comprise a helper lipid such as cholesterol, an optional neutral lipid such as DSPC, and a stealth lipid such as S010, S024, S027, S031, or S033.

Liposomes are amphiphilic lipids which can form bilayers in an aqueous environment to encapsulate an aqueous core. The polypeptide (e.g., Cas protein) or polynucleotide (e.g., guide RNA) may be incorporated into the aqueous core. These lipids can have an anionic, cationic or zwitterionic hydrophilic head group. Liposomes can be formed from a single lipid or from a mixture of lipids. A mixture may comprise (1) a mixture of anionic lipids; (2) a mixture of cationic lipids; (3) a mixture of zwitterionic lipids; (4) a mixture of anionic lipids and cationic lipids; (5) a mixture of anionic lipids and zwitterionic lipids; (6) a mixture of zwitterionic lipids and cationic lipids; or (7) a mixture of anionic lipids, cationic lipids and zwitterionic lipids. Similarly, a mixture may comprise both saturated and unsaturated lipids. Exemplary phospholipids include, but are not limited to, phosphatidylethanolamines, phosphatidylcholines, phosphatidylserines, and phosphatidylglycerols. Cationic lipids include, but are not limited to, 1,2-distearyloxy-N,N-dimethyl-3-aminopropane (DSDMA), dioleoyl trimethylammonium propane (DOTAP), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids include dodecylphosphocholine, DPPC, and DOPC.

The liposomes or LNPs may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes.

In some examples, the liposomes or LNPs comprise cationic lipids. In some examples, the liposomes or LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., WO 2019/067992, WO 2017/173054, WO 2015/095340, and WO 2014/136086, each of which is herein incorporated by reference in its entirety for all purposes. In some examples, the LNPs comprise molar ratios of a cationic lipid amine to RNA phosphate (N:P) of about 4.5, about 5.0, about 5.5, about 6.0, or about 6.5. In some examples, the terms cationic and ionizable in the context of LNP lipids are interchangeable (e.g., wherein ionizable lipids are cationic depending on the pH).

The lipid for encapsulation and endosomal escape can be a cationic lipid. The lipid can also be a biodegradable lipid, such as a biodegradable ionizable lipid. One example of a suitable lipid is Lipid A or LPO1, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl]4-(dimethylamino)butanoate or Dlin-MC3-DMA (MC3))).

Additional suitable cationic lipids include, but are not limited to 1,2-DiLinoleyloxy-N,N-dimethylaminopropane (DLinDMA), 1,2-Dilinolenyloxy-N,N-dimethylaminopropane (DLenDMA), dioctadecyldimethylammonium (DODMA), distearyldimethylammonium (DSDMA), N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N(N′,N′-dimethylaminoethane)-carbamoyl)cholesterol (DC-Chol), and N-(1,2-dimyristyloxyprop-3-yl)-N,N-dimethyl-N-hydroxyethyl ammonium bromide (DMRIE). For example, cationic lipids that have a positive charge at below physiological pH include, but are not limited to, DODAP, DODMA, and DMDMA. In some embodiments, the cationic lipids comprise C18 alkyl chains, ether linkages between the head group and alkyl chains, and 0 to 3 double bonds. Such lipids include, e.g., DSDMA, DLinDMA, DLenDMA, and DODMA. The cationic lipids may comprise ether linkages and pH titratable head groups. Such lipids include, e.g., DODMA. Additional cationic lipids are described in U.S. Pat. Nos. 7,745,651; 5,208,036; 5,264,618; 5,279,833; 5,283,185; 5,753,613; and 5,785,992, incorporated herein by reference.

In some embodiments, the cationic lipids may comprise a protonatable tertiary amine head group. Such lipids are referred to herein as ionizable lipids. Ionizable lipids refer to lipid species comprising an ionizable amine head group and typically comprising a pKa of less than about 7. In environments with an acidic pH, the ionizable amine head group is protonated such that the ionizable lipid preferentially interacts with negatively charged molecules (e.g., nucleic acids such as the recombinant polynucleotides described herein) thus facilitating liposome or LNP assembly and encapsulation. Therefore, in some embodiments, ionizable lipids can increase the loading of nucleic acids into liposomes or LNPs. In environments where the pH is greater than about 7 (e.g., physiologic pH of 7.4), the ionizable lipid comprises a neutral charge. When particles comprising ionizable lipids are taken up into the low pH environment of an endosome (e.g., pH<7), the ionizable lipid is again protonated and associates with the anionic endosomal membranes, promoting release of the contents encapsulated by the particle.

In some embodiments, the liposomes or LNPs may comprise one or more non-cationic helper lipids. Exemplary helper lipids include (1,2-dilauroyl-sn-glycero-3-phosphoethanolamine) (DLPE), 1,2-diphytanoyl-sn-glycero-3-phosphoethanolamine (D iPPE), 1,2-distearoyl-sn-glycero-3-phosphocholine (DSPC), 1,2-dipalmitoyl-sn-glycero-3-phosphocholine (DPPC), 1,2-dioleyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), (1,2-dioleoyl-sn-glycero-3-phospho-(1′-rac-glycerol) (DOPG), 1,2-distearoyl-sn-glycero-3-phosphoethanolamine (DSPE), ceramides, sphingomyelins, and cholesterol.

Some such lipids suitable for use in the liposomes or LNPs described herein are biodegradable in vivo. Examples of biodegradable lipids include, but are not limited to, (9Z,12Z)-3-((4,4-bis(octyloxy)butanoyl)oxy)-2-((((3-20 (diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl(9Z,12Z)-octadeca-9,12-dienoate) or another ionizable lipid. See, e.g., PCT Publication Nos. WO2017/173054, WO2015/095340, and WO2014/136086. In some embodiments, the term cationic and ionizable in the context of liposome or LNP lipids is interchangeable, e.g., wherein ionizable lipids are cationic depending on the pH.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the liposomes or LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the liposomes or LNPs. Suitable stealth lipids include lipids having a hydrophilic head group linked to a lipid moiety.

The hydrophilic head group of stealth lipid can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide. The term PEG means any polyethylene glycol or other polyalkylene ether polymer. In certain liposome or LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (I-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N—[methoxy(polyethylene glycol)-2000](PEG2k-DMG), 1,2-distearoyl-sn-glycero-3-phosphoethanolamine-N-[methoxy(polyethylene glycol)-2000](PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy(polyethylene glycol)-2000](PEG2k-DSA). In one particular example, the stealth lipid may be PEG2k-DMG.

In some embodiments, the liposomes or LNPs may further comprise one or more of PEG-modified lipids that comprise a poly(ethylene)glycol chain of up to 5 kDa in length covalently attached to a lipid comprising one or more C6-C20 alkyls. In some embodiments, the liposomes or LNPs further comprise 1,2-Distearoyl-sn-glycero-3-phosphoethanolamine-Poly(ethylene glycol) (DSPE-PEG), or 1,2-distearoyl-sn-glycero-3-phosphoethanolamine-N-[amino(polyethylene glycol)](DSPE-PEG-amine). In some embodiments, the PEG-modified lipid comprises about 0.1% to about 1% of the total lipid content in a lipid nanoparticle. In some embodiments, the PEG-modified lipid comprises about 0.1%, about 0.2% about 0.3%, about 0.4%, about 0.5%, about 0.6%, about 0.7%, about 0.8%, about 0.9%, or about 1.0%, of the total lipid content in the liposome or lipid nanoparticle.

In some embodiments, a liposome or LNP described herein may comprise a conjugated lipid that inhibits aggregation of lipid particles. Such lipid conjugates include, but are not limited to, PEG-lipid conjugates such as, e.g, PEG coupled to dialkyloxypropyls (e.g, PEG-DAA conjugates), PEG coupled to diacylglycerols (e.g, PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)—lipid conjugates (e.g., POZ-DAA conjugates), polyamide oligomers (e.g, ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain embodiments, non-ester containing linker moieties, such as amides or carbamates, are used.

The liposomes or LNPs can comprise different respective molar ratios of the component lipids in the formulation. The mol-% of the CCD lipid may be, for example, from about 30 mol-% to about 60 mol-%. The mol-% of the helper lipid may be, for example, from about 30 mol-% to about 60 mol-%. The mol-% of the neutral lipid may be, for example, from about 1 mol-% to about 20 mol-%. The mol-% of the stealth lipid may be, for example, from about 1 mol-% to about 10 mol-%

The liposomes or LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100. The N/P ratio can also be from about 4 to about 6.

In some embodiments, the liposome or LNP can comprise a nuclease agent (e.g., CRISPR/Cas system, ZFN, or TALEN), can comprise a polynucleotide molecule (e.g., guide RNA), can comprise a nucleic acid construct encoding a polypeptide of interest (e.g., multidomain therapeutic protein), or can comprise both a nuclease agent (e.g., a CRISPR/Cas system) and a nucleic acid construct encoding a polypeptide of interest (e.g., a donor template for use in gene editing). Regarding CRISPR/Cas systems, the liposomes or LNPs can comprise the Cas protein in any form (e.g., protein, DNA, or mRNA) and/or can comprise the guide RNA(s) in any form (e.g., DNA or RNA). In one example, the liposomes or LNPs comprise the Cas protein in the form of mRNA (e.g., a modified RNA as described herein) and the guide RNA(s) in the form of RNA (e.g., a modified guide RNA as disclosed herein). As another example, the liposomes or LNPs can comprise the Cas protein in the form of protein and the guide RNA(s) in the form of RNA). In one example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end and/or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity.

In certain liposomes or LNPs, the cargo can include a guide RNA or a nucleic acid encoding a guide RNA. In certain liposomes or LNPs, the cargo can include an mRNA encoding a Cas nuclease, such as Cas9, and a guide RNA or a nucleic acid encoding a guide RNA. In certain liposomes or LNPs, the cargo can include a nucleic acid construct encoding a polypeptide of interest (e.g., multidomain therapeutic protein) as described elsewhere herein. In certain liposomes or LNPs, the cargo can include an mRNA encoding a Cas nuclease, such as Cas9, a guide RNA or a nucleic acid encoding a guide RNA, and a nucleic acid construct encoding a polypeptide of interest (e.g., multidomain therapeutic protein). In some liposomes or LNPs, the lipid component comprises an amine lipid such as a biodegradable, ionizable lipid. In some instances, the lipid component comprises biodegradable, ionizable lipid, cholesterol, DSPC, and PEG-DMG. For example, Cas9 mRNA and gRNA can be delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG.

In some liposomes or LNPs, the cargo can comprise Cas mRNA (e.g., Cas9 mRNA) and gRNA. The Cas mRNA and gRNAs can be in different ratios. For example, the LNP formulation can include a ratio of Cas mRNA to gRNA nucleic acid ranging from about 25:1 to about 1:25. Alternatively, the liposome or LNP formulation can include a ratio of Cas mRNA to gRNA nucleic acid of from about 2:1 to about 1:2. In specific examples, the ratio of Cas mRNA to gRNA can be about 2:1.

In some liposomes or LNPs, the cargo can comprise a nucleic acid construct encoding a polypeptide of interest (e.g., multidomain therapeutic protein) and gRNA. The nucleic acid construct encoding a polypeptide of interest (e.g., multidomain therapeutic protein) and gRNAs can be in different ratios. For example, the liposome or LNP formulation can include a ratio of nucleic acid construct to gRNA nucleic acid ranging from about 25:1 to about 1:25.

A specific example of a suitable LNP has a nitrogen-to-phosphate (N/P) ratio of about 4.5 and contains biodegradable cationic lipid, cholesterol, DSPC, and PEG2k-DMG in an about 45:44:9:2 molar ratio (about 45:about 44:about 9:about 2). The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235, herein incorporated by reference in its entirety for all purposes. The Cas9 mRNA can be in an about 1:1 (about 1:about 1) ratio by weight to the guide RNA. Another specific example of a suitable LNP contains Dlin-MC3-DMA (MC3), cholesterol, DSPC, and PEG-DMG in an about 50:38.5:10:1.5 molar ratio (about 50:about 38.5:about 10:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP has a nitrogen-to-phosphate (N/P) ratio of about 6 and contains biodegradable cationic lipid, cholesterol, DSPC, and PEG2k-DMG in an about 50:38:9:3 molar ratio (about 50:about 38:about 9:about 3). The biodegradable cationic lipid can be Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 (about 2:about 1) ratio by weight to the guide RNA.

Another specific example of a suitable LNP has a nitrogen-to-phosphate (N/P) ratio of about 3 and contains a cationic lipid, a structural lipid, cholesterol (e.g., cholesterol (ovine) (Avanti 700000)), and PEG2k-DMG (e.g., PEG-DMG 2000 (NOF America-SUNBRIGHT® GM-020 (DMG-PEG)) in an about 50:10:38.5:1.5 ratio (about 50:about 10:about 38.5:about 1.5) or an about 47:10:42:1 ratio (about 47:about 10:about 42:about 1). The structural lipid can be, for example, DSPC (e.g., DSPC (Avanti 850365)), SOPC, DOPC, or DOPE. The cationic/ionizable lipid can be, for example, Dlin-MC3-DMA (e.g., Dlin-MC3-DMA (Biofine International)). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP contains Dlin-MC3-DMA, DSPC, cholesterol, and a PEG lipid in an about 45:9:44:2 ratio (about 45:about 9:about 44:about 2). Another specific example of a suitable LNP contains Dlin-MC3-DMA, DOPE, cholesterol, and PEG lipid or PEG DMG in an about 50:10:39:1 ratio (about 50:about 10:about 39:about 1). Another specific example of a suitable LNP has Dlin-MC3-DMA, DSPC, cholesterol, and PEG2k-DMG at an about 55:10:32.5:2.5 ratio (about 55:about 10:about 32.5:about 2.5). Another specific example of a suitable LNP has Dlin-MC3-DMA, DSPC, cholesterol, and PEG-DMG in an about 50:10:38.5:1.5 ratio (about 50:about 10:about 38.5:about 1.5). Another specific example of a suitable LNP has Dlin-MC3-DMA, DSPC, cholesterol, and PEG-DMG in an about 50:10:38.5:1.5 ratio (about 50:about 10:about 38.5:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Other examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes.

Dynamic Light Scattering (“DLS”) can be used to characterize the polydispersity index (“PDI”) and size of the liposomes and LNPs. In some embodiments, the PDI may range from about 0.005 to about 0.75. In some embodiments, the PDI may range from about 0.01 to about 0.5. In some embodiments, the PDI may range from about 0.02 to about 0.4. In some embodiments, the PDI may range from about 0.03 to about 0.35. In some embodiments, the PDI may range from about 0.1 to about 0.35.

The LNPs disclosed herein may have a size of about 1 to about 250 nm. In some embodiments, the LNPs may have a size of about 10 to about 200 nm. In some embodiments, the LNPs may have a size of about 20 to about 150 nm. In some embodiments, the LNPs may have a size of about 50 to about 150 nm. In some embodiments, the LNPs may have a size of about 50 to about 100 nm. In some embodiments, the LNPs may have a size of about 50 to about 120 nm. In some embodiments, the LNPs may have a size of about 75 to about 150 nm. In some embodiments, the LNPs may have a size of about 30 to about 200 nm. In some embodiments, the average sizes (diameters) of the fully formed nanoparticles are measured by dynamic light scattering on a Malvern Zetasizer (e.g., the nanoparticle sample may be diluted in phosphate buffered saline (PBS) so that the count rate is approximately 200-400 kcts, and the data may be presented as a weighted-average of the intensity measure).

In some embodiments, the liposomes or LNPs may be formed with an average encapsulation efficiency ranging from about 50% to about 100%. In some embodiments, the liposomes or LNPs may be formed with an average encapsulation efficiency ranging from about 50% to about 70%. In some embodiments, the liposomes or LNPs may be formed with an average encapsulation efficiency ranging from about 70% to about 90%. In some embodiments, the liposomes or LNPs may be formed with an average encapsulation efficiency ranging from about 90% to about 100%. In some embodiments, the liposomes or LNPs may be formed with an average encapsulation efficiency ranging from about 75% to about 95%.

In addition to liposomes and LNPs, an anti-TfR antigen-binding protein disclosed herein, such as an scFv or an antibody or an antigen-binding fragment thereof, may be conjugated to other carriers for delivery of nucleic acid and/protein molecules. Examples of other suitable carriers include, but are not limited to, lipoids and lipoplexes, particulate or polymeric nanoparticles, inorganic nanoparticles, peptide carriers, nanoparticle mimics, nanotubes, conjugates, immune stimulating complexes (ISCOM), virus-like particles (VLPs), self-assembling proteins, or emulsion delivery systems such as cationic submicron oil-in-water emulsions.

Polymeric microparticles or nanoparticles can also be used to encapsulate or adsorb a polypeptide (e.g., Cas protein) or polynucleotide (e.g., guide RNA). The particles may be substantially non-toxic and biodegradable. The particles useful for delivering a polynucleotide (e.g., guide RNA) may have an optimal size and zeta potential. For example, the microparticles may have a diameter in the range of 0.02 μm to 8 μm. In the instances when the composition has a population of micro- or nanoparticles with different diameters, at least 80%, 85%, 90%, or 95% of those particles ideally have diameters in the range of 0.03-7 μm. The particles may also have a zeta potential of between 40-100 mV, in order to provide maximal adsorption of the polynucleotide (e.g., guide RNA) to the particles.

Non-toxic and biodegradable polymers include, but are not limited to, poly(ahydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, one or more natural polymers such as a polysaccharide, for example pullulan, alginate, inulin, and chitosan, and combinations thereof. In some embodiments, the particles are formed from poly(ahydroxy acids), such as a poly(lactides) (PLA), poly(g-glutamic acid) (g-PGA), poly(ethylene glycol) (PEG), polystyrene, copolymers of lactide and glycolide such as a poly(D,L-lactide-co-glycolide) (PLG), and copolymers of D,L-lactide and caprolactone. Useful PLG polymers can include those having a lactide/glycolide molar ratio ranging, for example, from 20:80 to 80:20 e.g., 25:75, 40:60, 45:55, 55:45, 60:40, 75:25. Useful PLG polymers include those having a molecular weight between, for example, 5,000-200,000 Da e.g., between 10,000-100,000, 20,000-70,000, 40,000-50,000 Da.

The polymeric nanoparticle may also form hydrogel nanoparticles, hydrophilic three-dimensional polymer networks with favorable properties including flexible mesh size, large surface area for multivalent conjugation, high water content, and high loading capacity for antigens. Polymers such as Poly(L-lactic acid) (PLA), PLGA, PEG, and polysaccharides are suitable for forming hydrogel nanoparticles.

For example, the inorganic nanoparticles may be calcium phosphate nanoparticles, silicon nanoparticles or gold nanoparticles. Inorganic nanoparticles typically have a rigid structure and comprise a shell in which a polypeptide or polynucleotide is encapsulated or a core to which the polypeptide or polynucleotide may be covalently attached. The core may comprise one or more atoms such as gold (Au), silver (Ag), copper (Cu) atoms, Au/Ag, Au/Cu, Au/Ag/Cu, Au/Pt, Au/Pd or Au/Ag/Cu/Pd or calcium phosphate (CaP).

Other molecules suitable for complexing with the polypeptides or polynucleotides of the disclosure include cationic molecules, such as, polyamidoamine, dendritic polylysine, polyethylene irinine or polypropylene imine, polylysine, chitosan, DNA-gelatin coarcervates, DEAE dextran, dendrimers, or polyethylenimine (PEI).

In some embodiments, polypeptides or polynucleotides of the present disclosure can be conjugated to nanoparticles. Nanoparticles that may be used for conjugation with antigens and/or antibodies of the present disclosure include but not are limited to chitosan-shelled nanoparticles, carbon nanotubes, PEGylated liposomes, poly(d,1-lactide-co-glycolide)/montmorillonite (PLGA/MMT) nanoparticles, poly(lactide-co-glycolide) (PLGA) nanoparticles, poly-(malic acid)-based nanoparticles, and other inorganic nanoparticles (e.g., nanoparticles made of magnesium-aluminium layered double hydroxides with disuccinimidyl carbonate (DSC), and TiO₂ nanoparticles). Nanoparticles can be developed and conjugated to an antigens and/or antibodies contained in a composition for targeting virus-infected cells.

Oil-in-water emulsions may also be used for delivering a polypeptide or polynucleotide (e.g., mRNA) to a subject. Examples of oils useful for making the emulsions include animal (e.g., fish) oil or vegetable oil (e.g., nuts, grains and seeds). The oil may be biodegradable and biocompatible. Exemplary oils include, but are not limited to, tocopherols and squalene, a shark liver oil which is a branched, unsaturated terpenoid and combinations thereof. Terpenoids are branched chain oils that are synthesized biochemically in 5-carbon isoprene units.

The aqueous component of the emulsion can be water or can be water in which additional components have been added. For example, it may include salts to form a buffer e.g., citrate or phosphate salts, such as sodium salts. Exemplary buffers include a borate buffer, a citrate buffer, a histidine buffer a phosphate buffer, a Tris buffer, or a succinate buffer.

In some embodiments, the oil-in water emulsions include one or more cationic molecules. For example, a cationic lipid can be included in the emulsion to provide a positively charged droplet surface to which negatively-charged polynucleotide (e.g., mRNA) can attach. Exemplary cationic lipids include, but are not limited to: 1,2-dioleoyloxy-3-(trimethylammonio)propane (DOTAP), 1,2-Dimyristoyl-3-Trimethyl-AmmoniumPropane (DMTAP), 3′-[N—(N′,N′-Dimethylaminoethane)-carbamoyl]Cholesterol (DC Cholesterol), dimethyldioctadecyl-ammonium (DDA e.g., the bromide), dipalmitoyl(C16:0)trimethyl ammonium propane (DPTAP), distearoyltrimethylammonium propane (DSTAP). Other useful cationic lipids include benzalkonium chloride (BAK), benzethonium chloride, cholesterol hemisuccinate choline ester, lipopolyamines (e.g., dioctadecylamidoglycylspermine (DOGS), dipalmitoyl phosphatidylethanol-amidospermine (DPPES)), cetramide, cetylpyridinium chloride (CPC), cetyl trimethylammonium chloride (CTAC), cationic derivatives of cholesterol (e.g., cholesteryl-3.beta.-oxysuccinamidoethylenetrimethylammonium salt, cholesteryl-3.beta.-oxysuccinamidoethylene-dimethylamine, cholesteryl-3.beta.-carboxyamidoethylenetrimethylammonium salt, and cholesteryl-3.beta.-carboxyamidoethylenedimethylamine), N,N′,N′-polyoxyethylene (10)-N-tallow-1,3-diaminopropane, dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemetha-naminium chloride (DEBDA), cholesteryl (4′-trimethylammonio) butanoate), N-alkyl pyridinium salts (e.g., cetylpyridinium bromide and cetylpyridinium chloride), N-alkylpiperidinium salts, dicationic bolaform electrolytes (C12Me6; C12BU6), dialkylglycetylphosphorylcholine, lysolecithin, L-alpha.dioleoylphosphatidylethanolamine, lipopoly-L (or D)-lysine (LPLL, LPDL), poly(L (or D)-lysine conjugated to N-glutarylphosphatidylethanolamine, dialkyldimethylammonium salts, [1-(2,3-dioleyloxy)-propyl]-N,N,N,trimethylammonium chloride, 1,2-diacyl-3-(trimethylammonio) propane (acyl group can be dimyristoyl, dipalmitoyl, distearoyl, or dioleoyl), 1,2-diacyl-3 (dimethylammonio)propane (acyl group can be dimyristoyl, dipalmitoyl, distearoyl, or dioleoyl), 1,2-dioleoyl-3-(4′-trimethyl-ammonio)butanoyl-sn-glycerol, 1,2-dioleoyl 3-succinyl-sn-glycerol choline ester, didodecyl glutamate ester with pendant amino group (C GluPhCnN), and ditetradecyl glutamate ester with pendant amino group (C14GluCnN+).

In some embodiments, in addition to the oil and cationic lipid, an emulsion can also include a non-ionic surfactant and/or a zwitterionic surfactant. Examples of useful surfactants include, but are not limited to: the polyoxyethylene sorbitan esters surfactants, e.g., polysorbate 20 and polysorbate 80; copolymers of ethylene oxide, propylene oxide, and/or butylene oxide, linear block copolymers; phospholipids, e.g., phosphatidylcholine; polyoxyethylene fatty ethers derived from lauryl, cetyl, stearyl and oleyl alcohols; polyoxyethylene-9-lauryl ether; octoxynols; (octylphenoxy)polyethoxyethanol; and sorbitan esters.

In some embodiments, a polynucleotide described herein may be incorporated into polynucleotide complexes, such as, but not limited to, nanoparticles (e.g., polynucleotide self-assembled nanoparticles, polymer-based self-assembled nanoparticles, inorganic nanoparticles, lipid nanoparticles, semiconductive/metallic nanoparticles), gels and hydrogels, polynucleotide complexes with cations and anions, microparticles, and any combination thereof. The polynucleotide complexes may be conjugated to an anti-TfR antigen-binding protein described herein, e.g., via linkage to the polynucleotide or nanoparticle/hydrogel/microparticle.

In some embodiments, the polynucleotides disclosed herein may be formulated as self-assembled nanoparticles. As a non-limiting example, polynucleotides may be used to make nanoparticles which may be used in a delivery system for the polynucleotides (See e.g., PCT Publication No. WO2012/125987). In some embodiments, the polynucleotide self-assembled nanoparticles may comprise a core of the polynucleotides disclosed herein and a polymer shell. The polymer shell may be any of the polymers described herein and are known in the art. In an additional embodiment, the polymer shell may be used to protect the polynucleotides in the core.

In some embodiments, self-assembled nanoparticles may be microsponges formed of long polymers of polynucleotide hairpins which form into crystalline “pleated” sheets before self-assembling into microsponges. These microsponges are densely-packed sponge like microparticles which may function as an efficient carrier and may be able to deliver cargo to a cell. The microsponges may be from 1 μm to 300 nm in diameter. The microsponges may be complexed with other agents known in the art to form larger microsponges. As a non-limiting example, the microsponge may be complexed with an agent to form an outer layer to promote cellular uptake such as polycation polyethyleneime (PEI). This complex can form a 250-nm diameter particle that can remain stable at high temperatures (150° C.) (Grabow and Jaegar, Nature Materials 2012, 11:269-269). Additionally, these microsponges may be able to exhibit an extraordinary degree of protection from degradation by ribonucleases. In an embodiment, the polymer-based self-assembled nanoparticles such as, but not limited to, microsponges, may be fully programmable nanoparticles. The geometry, size and stoichiometry of the nanoparticle may be precisely controlled to create the optimal nanoparticle for delivery of cargo such as, but not limited to, polynucleotides.

In some embodiments, a polynucleotide disclosed herein may be formulated in inorganic nanoparticles (see U.S. Pat. No. 8,257,745). The inorganic nanoparticles may include, but are not limited to, clay substances that are water swellable. As a non-limiting example, the inorganic nanoparticle may include synthetic smectite clays which are made from simple silicates (See U.S. Pat. Nos. 5,585,108 and 8,257,745).

In some embodiments, a polynucleotide disclosed herein may be formulated in water-dispersible nanoparticle comprising a semiconductive or metallic material (U.S. Patent Application Publication No. 2012/0228565; herein incorporated by reference in its entirety) or formed in a magnetic nanoparticle (U.S. Patent Application Publication No. 2012/0265001 and 2012/0283503). The water-dispersible nanoparticles may be hydrophobic nanoparticles or hydrophilic nanoparticles.

In some embodiments, the polynucleotides disclosed herein may be encapsulated into any hydrogel known in the art which may form a gel when injected into a subject. Hydrogels are a network of polymer chains that are hydrophilic, and are sometimes found as a colloidal gel in which water is the dispersion medium. Hydrogels are highly absorbent (they can contain over 99% water) natural or synthetic polymers. Hydrogels also possess a degree of flexibility very similar to natural tissue, due to their significant water content. The hydrogel described herein may be used to encapsulate lipid nanoparticles which are biocompatible, biodegradable and/or porous.

As a non-limiting example, the hydrogel may be an aptamer-functionalized hydrogel. The aptamer-functionalized hydrogel may be programmed to release one or more polynucleotides using polynucleotide hybridization. (Battig et al., J. Am. Chem. Society. 2012 134:12410-12413). In some embodiments, the polynucleotide may be encapsulated in a lipid nanoparticle and then the lipid nanoparticle may be encapsulated into a hydrogel.

In some embodiments, the polynucleotides disclosed herein may be encapsulated into a fibrin gel, fibrin hydrogel or fibrin glue. In another embodiment, the polynucleotides may be formulated in a lipid nanoparticle or a rapidly eliminated lipid nanoparticle prior to being encapsulated into a fibrin gel, fibrin hydrogel or a fibrin glue. In yet another embodiment, the polynucleotides may be formulated as a lipoplex prior to being encapsulated into a fibrin gel, hydrogel or a fibrin glue. Fibrin gels, hydrogels and glues comprise two components, a fibrinogen solution and a thrombin solution which is rich in calcium (See e.g., Spicer and Mikos, Journal of Controlled Release 2010. 148: 49-55; Kidd et al. Journal of Controlled Release 2012. 157:80-85). The concentration of the components of the fibrin gel, hydrogel and/or glue can be altered to change the characteristics, the network mesh size, and/or the degradation characteristics of the gel, hydrogel and/or glue such as, but not limited to changing the release characteristics of the fibrin gel, hydrogel and/or glue. (See e.g., Spicer and Mikos, Journal of Controlled Release 2010. 148: 49-55; Kidd et al. Journal of Controlled Release 2012. 157:80-85; Catelas et al. Tissue Engineering 2008. 14:119-128). This feature may be advantageous when used to deliver the polynucleotide disclosed herein. (See e.g., Kidd et al. Journal of Controlled Release 2012. 157:80-85; Catelas et al. Tissue Engineering 2008. 14:119-128).

In some embodiments, a polynucleotide disclosed herein may include cations or anions. In one embodiment, the formulations include metal cations such as, but not limited to, Zn²⁺, Ca²⁺, Cu²⁺, Mg²⁺ and combinations thereof. As a non-limiting example, formulations may include polymers and a polynucleotide complexed with a metal cation (See U.S. Pat. Nos. 6,265,389 and 6,555,525).

In some embodiments, a polynucleotide may be formulated in nanoparticles and/or microparticles. These nanoparticles and/or microparticles may be molded into any size shape and chemistry. As an example, the nanoparticles and/or microparticles may be made using the PRINT@technology by LIQUIDA TECHNOLOGIES (Morrisville, N.C.) (See e.g., International Pub. Publication No. WO2007/024323).

In some embodiments, the polynucleotides disclosed herein may be formulated in NanoJackets and NanoLiposomes by Keystone Nano (State College, Pa.). NanoJackets are made of compounds that are naturally found in the body including calcium, phosphate and may also include a small amount of silicates. Nanojackets may range in size from 5 to 50 nm and may be used to deliver hydrophilic and hydrophobic compounds such as, but not limited to, polynucleotides, primary constructs and/or polynucleotide. NanoLiposomes are made of lipids such as, but not limited to, lipids which naturally occur in the body. NanoLiposomes may range in size from 60-80 nm and may be used to deliver hydrophilic and hydrophobic compounds such as, but not limited to, polynucleotides, primary constructs and/or polynucleotide. In one aspect, the polynucleotides disclosed herein are formulated in a NanoLiposome such as, but not limited to, Ceramide NanoLiposomes.

### Gene Editing System

In various embodiments, a molecular cargo described herein can include a gene editing system or components of such systems. Various known gene editing systems can be used in the methods and compositions described herein, including, e.g., a Clustered Regularly Interspersed Short Palindromic Repeats (CRISPR)/Cas system; zinc finger nuclease (ZFN) system; transcription activator-like effector nuclease (TALEN) system, or systems using meganucleases, restriction endonucleases, or recombinases. Generally, these gene editing systems are used to modify a genome within a cell by inducing a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA (gRNA) to guide specific cleavage or nicking of a target DNA sequence. Further, targeted nucleases have been developed, and additional nucleases are being developed, for example based on the Argonaute system (e.g., from T. thermophilus, known as ‘TtAgo’, see Swarts et al (2014) Nature 507(7491): 258-261), which also may have the potential for uses in genome editing and gene therapy.

Deletion of DNA may be performed using a gene editing system to knock-out or disrupt a target gene. A knock-out can be a gene knock-down or the gene can be knocked out by a mutation such as, a point mutation, an insertion, a deletion, a frameshift, or a missense mutation by techniques known in the art. Alternatively, a knock-in of an exogenous gene or replacement of a defective gene with a corrective gene can also be achieved with a gene editing system. In such instances, a donor template carrying an heterologous gene to be inserted into a genomic locus is provided along with a gene editing system. The donor template would typically include homology arms corresponding to the genomic locus which is targeted by a gene editing system.

There are various ways to incorporate a gene editing system or component(s) thereof (e.g., Cas protein, guide RNA) to an anti-TfR protein-drug conjugate described herein. In some embodiments, a gene editing system or component(s) thereof (e.g., Cas protein or nucleic acid (e.g., mRNA or DNA) encoding the Cas protein, guide RNA or a DNA encoding the guide RNA) are loaded to a carrier described, such as a liposome or LNP, which is conjugated to an anti-TfR antigen-binding protein described herein. In some embodiments, a guide RNA or a DNA encoding the guide RNA is conjugated to an anti-TfR antigen-binding protein described herein. In some embodiments, a gene editing nuclease (e.g., Cas protein, ZFN, TALEN) or one or more nucleic acids (e.g., mRNA or DNA) encoding the gene editing nuclease is conjugated to anti-TfR antigen-binding protein described herein. In some embodiments, both a guide RNA (or DNA encoding the guide DNA) and a Cas protein (or nucleic acid (e.g., mRNA or DNA) encoding the Cas protein) may be conjugated to an anti-TfR antigen-binding protein described herein. In some embodiments, a guide RNA (or DNA encoding the guide RNA) is conjugated to an anti-TfR antigen-binding protein described herein, and a Cas protein (or nucleic acid (e.g., mRNA or DNA) encoding the Cas protein) is loaded to a carrier described, such as a liposome or LNP, which is conjugated to an anti-TfR antigen-binding protein described herein. In some embodiments, a Cas protein (or nucleic acid (e.g., mRNA or DNA) encoding the Cas protein) is conjugated to an anti-TfR antigen-binding protein described herein, and a guide RNA (or DNA encoding the guide RNA) is loaded to a carrier described, such as a liposome or LNP, which is conjugated to an anti-TfR antigen-binding protein described herein.

In some embodiments, the molecular cargo disclosed herein can comprise a CRISPR/Cas system or components of such systems. CRISPR/Cas systems include transcripts and other elements involved in the expression of, or directing the activity of, Cas genes. A CRISPR/Cas system can be, for example, a type I, a type II, or a type III system. Alternatively, a CRISPR/Cas system can be a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed cleavage of nucleic acids.

Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (e.g., assigned UniProt accession number Q99ZW2) is an exemplary Cas9 protein. Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacterjejuni (CjCas9) (e.g., assigned UniProt accession number Q0P897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, WO 2019/067910, WO 2020/082042, US 2020/0270617, WO 2020/082041, US 2020/0268906, WO 2020/082046, and US 2020/0289628, each of which is herein incorporated by reference in its entirety for all purposes. Specific examples of ORFs and Cas9 amino acid sequences are provided in Table 30 at paragraph [0449] WO 2019/067910, and specific examples of Cas9 mRNAs and ORFs are provided in paragraphs [0214]-[0234] of WO 2019/067910. See also WO 2020/082046 A2 (pp. 84-85) and Table 24 in WO 2020/069296, each of which is herein incorporated by reference in its entirety for all purposes.

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number AOQ7Q2) is an exemplary Cpf1 protein.

Another example of a Cas protein is CasX (Cas12e). CasX is an RNA-guided DNA endonuclease that generates a staggered double-strand break in DNA. CasX is less than 1000 amino acids in size. Exemplary CasX proteins are from Deltaproteobacteria (DpbCasX or DpbCas12e) and Planctomycetes (PlmCasX or PlmCas12e). Like Cpf1, CasX uses a single RuvC active site for DNA cleavage. See, e.g., Liu et al. (2019) Nature 566(7743):218-223, herein incorporated by reference in its entirety for all purposes.

Another example of a Cas protein is CasΦ (CasPhi or Cas12j), which is uniquely found in bacteriophages. CasΦ is less than 1000 amino acids in size (e.g., 700-800 amino acids). CasΦ cleavage generates staggered 5′ overhangs. A single RuvC active site in CasΦ is capable of crRNA processing and DNA cutting. See, e.g., Pausch et al. (2020) Science 369(6501):333-337, herein incorporated by reference in its entirety for all purposes.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Likewise, CasX and CasΦ generally comprise a single RuvC-like domain that cleaves both strands of a target DNA. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more or all of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If both of the nuclease domains are deleted or mutated, the resulting Cas protein (e.g., Cas9) will have a reduced ability to cleave both strands of a double-stranded DNA (e.g., a nuclease-null or nuclease-inactive Cas protein, or a catalytically dead Cas protein (dCas)). If none of the nuclease domains is deleted or mutated in a Cas9 protein, the Cas9 protein will retain double-strand-break-inducing activity. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes. If all of the nuclease domains are deleted or mutated in a Cas protein (e.g., both of the nuclease domains are deleted or mutated in a Cas9 protein), the resulting Cas protein (e.g., Cas9) will have a reduced ability to cleave both strands of a double-stranded DNA (e.g., a nuclease-null or nuclease-inactive Cas protein). One specific example is a D10A/H840A S. pyogenes Cas9 double mutant or a corresponding double mutant in a Cas9 from another species when optimally aligned with S. pyogenes Cas9. Another specific example is a D10A/N863A S. pyogenes Cas9 double mutant or a corresponding double mutant in a Cas9 from another species when optimally aligned with S. pyogenes Cas9.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) or a substitution at position D10 (e.g., D10A substitution) to generate a Cas nickase. See, e.g., WO 2016/106236, herein incorporated by reference in its entirety for all purposes. Examples of inactivating mutations in the catalytic domains of Nme2Cas9 are also known (e.g., D16A or H588A). Examples of inactivating mutations in the catalytic domains of St1Cas9 are also known (e.g., D9A, D598A, H599A, or N622A). Examples of inactivating mutations in the catalytic domains of St3Cas9 are also known (e.g., D10A or N870A). Examples of inactivating mutations in the catalytic domains of GjCas9 are also known (e.g., combination of D8A or H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Examples of inactivating mutations in the catalytic domains of CasX proteins are also known. With reference to CasX proteins from Deltaproteobacteria, D672A, E769A, and D935A (individually or in combination) or corresponding positions in other CasX orthologs are inactivating. See, e.g., Liu et al. (2019) Nature 566(7743):218-223, herein incorporated by reference in its entirety for all purposes.

Examples of inactivating mutations in the catalytic domains of CasΦ proteins are also known. For example, D371A and D394A, alone or in combination, are inactivating mutations. See, e.g., Pausch et al. (2020) Science 369(6501):333-337, herein incorporated by reference in its entirety for all purposes.

Cas proteins can also be operably linked to heterologous polypeptides as fusion proteins. For example, a Cas nuclease can be fused to a cleavage domain, an epigenetic modification domain, a transcriptional activation domain, or a transcriptional repressor domain. See WO 2014/089290, herein incorporated by reference in its entirety for all purposes. Examples of transcriptional activation domains include a herpes simplex virus VP 16 activation domain, VP64 (which is a tetrameric derivative of VP 16), a NFKB p65 activation domain, p53 activation domains 1 and 2, a CREB (cAMP response element binding protein) activation domain, an E2A activation domain, and an NFAT (nuclear factor of activated T-cells) activation domain. Other examples include activation domains from Octl, Oct-2A, SP1, AP-2, CTF1, P300, CBP, PCAF, SRC1, PvALF, ERF-2, OsGAI, HALF-1, C1, API, ARF-5, ARF-6, ARF-7, ARF-8, CPRF1, CPRF4, MYC-RP/GP, TRAB1PC4, and HSF1. See, e.g., US 2016/0237456, EP3045537, and WO 2011/146121, each of which is incorporated by reference in its entirety for all purposes.

In some cases, a transcriptional activation system can be used comprising a dCas9-VP64 fusion protein paired with MS2-p65-HSFI. Guide RNAs in such systems can be designed with aptamer sequences appended to sgRNA tetraloop and stem-loop 2 designed to bind dimerized MS2 bacteriophage coat proteins. See, e.g., Konermann et al. (2015) Nature 517(7536):583-588, herein incorporated by reference in its entirety for all purposes.

Examples of transcriptional repressor domains include inducible cAMP early repressor (ICER) domains, Kruppel-associated box A (KRAB-A) repressor domains, YY 1 glycine rich repressor domains, Spl-like repressors, E(spl) repressors, IKB repressor, and MeCP2. Other examples include transcriptional repressor domains from A/B, KOX, TGF-beta-inducible early gene (TIEG), v-erbA, SID, SID4X, MBD2, MBD3, DNMT1, DNMG3A, DNMT3B, Rb, ROM2, See, e.g., EP3045537 and WO 2011/146121, each of which is incorporated by reference in its entirety for all purposes. Cas nucleases can also be fused to a heterologous polypeptide providing increased or decreased stability. The fused domain or heterologous polypeptide can be located at the N-terminus, the C-terminus, or internally within the Cas nuclease.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 436) or PKKKRRV (SEQ ID NO: 437). The NLS may be a bipartite sequence, such as the NLS of nucleoplasmin, KRPAATKKAGQAKKKK (SEQ ID NO: 428). In a specific example, a single PKKKRKV (SEQ ID NO: 436) NLS may be linked at the C-terminus of the Cas protein. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge). Examples of codon-optimized Cas9 coding sequences, Cas9 mRNAs, and Cas9 protein sequences include those described in WO2013/176772, WO2014/065596, WO2016/106121, and WO2019/067910 are hereby incorporated by reference. In particular, the Cas9 coding sequences and Cas9 amino acid sequences of the table at paragraph [0449] WO2019/067910, and the Cas9 mRNAs and coding sequences of paragraphs [0214]-[0234] of WO2019/067910 are hereby incorporated by reference. When a nucleic acid encoding the Cas protein is introduced into the cell, the Cas protein can be transiently, conditionally, or constitutively expressed in the cell.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery. In preferred embodiments, promotors are accepted by regulatory authorities for use in humans. In certain embodiments, promotors drive expression in a liver cell.

Different promoters can be used to drive Cas expression or Cas9 expression. In some methods, small promoters are used so that the Cas or Cas9 coding sequence can fit into an AAV construct. For example, Cas or Cas9 and one or more gRNAs (e.g., 1 gRNA or 2 gRNAs or 3 gRNAs or 4 gRNAs) can be delivered via LNP-mediated delivery (e.g., in the form of RNA). Different promoters can be used to drive expression of the gRNA, such as a U6 promoter or the small tRNA Gln. Likewise, different promoters can be used to drive Cas9 expression.

Cas proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding Cas proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding Cas proteins can also be polyadenylated (to comprise a poly(A) tail). mRNA encoding Cas proteins can also be modified to include pseudouridine (e.g., can be fully substituted with pseudouridine). As another example, capped and polyadenylated Cas mRNA containing N1-methyl pseudouridine can be used. As another example, Cas mRNA fully substituted with pseudouridine can be used (i.e., all standard uracil residues are replaced with pseudouridine, a uridine isomer in which the uracil is attached with a carbon-carbon bond rather than nitrogen-carbon). Likewise, Cas mRNAs can be modified by depletion of uridine using synonymous codons. For example, capped and polyadenylated Cas mRNA fully substituted with pseudouridine can be used.

Cas mRNAs can comprise a modified uridine at least at one, a plurality of, or all uridine positions. The modified uridine can be a uridine modified at the 5 position (e.g., with a halogen, methyl, or ethyl). The modified uridine can be a pseudouridine modified at the 1 position (e.g., with a halogen, methyl, or ethyl). The modified uridine can be, for example, pseudouridine, N1-methyl-pseudouridine, 5-methoxyuridine, 5-iodouridine, or a combination thereof. In some examples, the modified uridine is 5-methoxyuridine. In some examples, the modified uridine is 5-iodouridine. In some examples, the modified uridine is pseudouridine. In some examples, the modified uridine is N1-methyl-pseudouridine. In some examples, the modified uridine is a combination of pseudouridine and N1-methyl-pseudouridine. In some examples, the modified uridine is a combination of pseudouridine and 5-methoxyuridine. In some examples, the modified uridine is a combination of N1-methyl pseudouridine and 5-methoxyuridine. In some examples, the modified uridine is a combination of 5-iodouridine and N1-methyl-pseudouridine. In some examples, the modified uridine is a combination of pseudouridine and 5-iodouridine. In some examples, the modified uridine is a combination of 5-iodouridine and 5-methoxyuridine.

Cas mRNAs disclosed herein can also comprise a 5′ cap, such as a Cap0, Cap1, or Cap2. A 5′ cap is generally a 7-methylguanine ribonucleotide (which may be further modified, e.g., with respect to ARCA) linked through a 5′-triphosphate to the 5′ position of the first nucleotide of the 5′-to-3′ chain of the mRNA (i.e., the first cap-proximal nucleotide). In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc. Natl. Acad. Sci. U.S.A. 111(33):12025-30 and Abbas et al. (2017) Proc. Natl. Acad. Sci. U.S.A. 114(11):E2106-E2115, each of which is herein incorporated by reference in its entirety for all purposes. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as non-self by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al. (2001) RNA 7:1486-1495, herein incorporated by reference in its entirety for all purposes.

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo and Moss (1990) Proc. Natl. Acad. Sci. U.S.A. 87:4023-4027 and Mao and Shuman (1994) J. Biol. Chem. 269:24472-24479, each of which is herein incorporated by reference in its entirety for all purposes.

Cas mRNAs can further comprise a poly-adenylated (poly-A or poly(A) or poly-adenine) tail. The poly-A tail can, for example, comprise at least 20, at least 30, at least 40, at least 50, at least 60, at least 70, at least 80, at least 90, or at least 100 adenines, and optionally up to 300 adenines. For example, the poly-A tail can comprise 95, 96, 97, 98, 99, or 100 adenine nucleotides.

In some embodiments, a CRISPR/Cas system can be used to create a site of insertion at a desired locus within a host genome, at which site a construct disclosed herein can be inserted to express one or more polypeptides of interest. Methods of designing suitable guide RNAs that target any desired locus of a host genome for insertion are well known in the art. A construct comprising a transgene may be heterologous with respect to its insertion site, for example, insertion of a heterologous transgene into a “safe harbor” locus. A construct comprising a transgene may be non-heterologous with respect to its insertion site, for example, insertion of a wild-type transgene into its endogenous locus.

Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences.

Such safe harbor loci can offer an open chromatin configuration in all tissues and can be ubiquitously expressed during embryonic development and in adults. See, e.g., Zambrowicz et al. (1997) Proc. Natl. Acad. Sci. U.S.A. 94:3789-3794, herein incorporated by reference in its entirety for all purposes. In addition, the safe harbor loci can be targeted with high efficiency, and safe harbor loci can be disrupted with no overt phenotype. Examples of safe harbor loci include ALB, CCR5, HPRT, AAVS1 (PPP1 R12C), Rosa (e.g., Rosa26), AngptiS, ApoC3, ASGR2, FIX (F9), G6PC, Gys2, HGD, Lp(a), Pcsk9, SERPINA1, TF, and TTR. See, e.g., U.S. Pat. Nos. 7,888,121; 7,972,854; 7,914,796; 7,951,925; 8,110,379; 8,409,861; 8,586,526; and US Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes. Other examples of target genomic loci include an ALB locus, a EESYR locus, a SARS locus, position 188,083,272 of human chromosome 1 or its non-human mammalian orthologue, position 3,046,320 of human chromosome 10 or its non-human mammalian orthologue, position 67, 328,980 of human chromosome 17 or its non-human mammalian orthologue, an adeno-associated virus site 1 (AAVS1) on chromosome, a naturally occurring site of integration of AAV virus on human chromosome 19 or its non-human mammalian orthologue, a chemokine receptor 5 (CCR5) gene, a chemokine receptor gene encoding an HIV-1 coreceptor, or a mouse Rosa26 locus or its non-murine mammalian orthologue.

In some embodiments, the heterologous gene may be inserted into a safe harbor locus and use the safe harbor locus's endogenous signal sequence. In some embodiments, the heterologous gene may comprise its own signal sequence, may be inserted into the safe harbor locus, and may further use the safe harbor locus's endogenous signal sequence. In some embodiments, the gene may comprise its own signal sequence and an internal ribosomal entry site (IRES), may be inserted into the safe harbor locus, and may further use the safe harbor locus's endogenous signal sequence. In some embodiments, the gene may comprise its own signal sequence and IRES, may be inserted into the safe harbor locus, and does not use the safe harbor locus's endogenous signal sequence. In some embodiments, the gene may be inserted into the safe harbor locus and may comprise an IRES and does not use any signal sequence.

In some methods, two or more nuclease agents can be used. For example, two or more nuclease agents can be used, each targeting a nuclease target sequence including or proximate to the start codon. As another example, two nuclease agents can be used, one targeting a nuclease target sequence including or proximate to the start codon, and one targeting a nuclease target sequence including or proximate to the stop codon, wherein cleavage by the nuclease agents can result in deletion of the coding region between the two nuclease target sequences. As yet another example, three or more nuclease agents can be used, with one or more (e.g., two) targeting nuclease target sequences including or proximate to the start codon, and one or more (e.g., two) targeting nuclease target sequences including or proximate to the stop codon, wherein cleavage by the nuclease agents can result in deletion of the coding region between the nuclease target sequences including or proximate to the start codon and the nuclease target sequence including or proximate to the stop codon.

In some embodiments, CRISPR/Cas systems used in the compositions and methods disclosed herein can be non-naturally occurring.

In some embodiments, the Cas protein (e.g., Cas9) may be complexed with a gRNA to form a ribonucleoprotein complex (RNP). In some embodiments, a molecular cargo (e.g., liposome or LNP) described herein comprises a ribonucleoprotein complex (RNP) comprising a Cas protein (e.g., Cas9) and a gRNA.

In some embodiments, a molecular cargo (e.g., liposomes and LNPs) described herein may comprise one or more components from gene editing systems other than a CRISPR/Cas system. In some embodiments, the molecular cargo is a nuclease, such as Zinc-finger nuclease (ZFN) or a TALEN, which is effective to bind and modify at a target gene.

Any nuclease molecular cargo that induces a nick or double-strand break into a desired target sequence or any DNA-binding protein that binds to a desired target sequence can be used in the methods and compositions disclosed herein. A naturally occurring or native nuclease molecular cargo can be employed so long as the nuclease molecular cargo induces a nick or double-strand break in a desired target sequence. Likewise, a naturally occurring or native DNA-binding protein can be employed so long as the DNA-binding protein binds to the desired target sequence. Alternatively, a modified or engineered nuclease molecular cargo or DNA-binding protein can be employed. An “engineered nuclease molecular cargo or DNA-binding protein” includes a nuclease molecular cargo or DNA-binding protein that is engineered (modified or derived) from its native form to specifically recognize a desired target sequence. Thus, an engineered nuclease molecular cargo or DNA-binding protein can be derived from a native, naturally occurring nuclease molecular cargo or DNA-binding protein or it can be artificially created or synthesized. The engineered nuclease molecular cargo or DNA-binding protein can recognize a target sequence, for example, wherein the target sequence is not a sequence that would have been recognized by a native (non-engineered or non-modified) nuclease molecular cargo or DNA-binding protein. The modification of the nuclease molecular cargo or DNA-binding protein can be as little as one amino acid in a protein cleavage molecular cargo or one nucleotide in a nucleic acid cleavage molecular cargo. Producing a nick or double-strand break in a target sequence or other DNA can be referred to herein as “cutting” or “cleaving” the target sequence or other DNA.

Active variants and fragments of nuclease molecular cargoes or DNA-binding proteins (i.e., an engineered nuclease molecular cargo or DNA-binding protein) are also provided. Such active variants can comprise at least 65%, 70%, 75%, 80%, 85%, 90%, 91%, 92%, 93%, 94%, 95%, 96%, 97%, 98%, 99% or more sequence identity to the native nuclease molecular cargo or DNA-binding protein, wherein the active variants retain the ability to cut at a desired target sequence and hence retain nick or double-strand-break-inducing activity or retain the ability to bind a desired target sequence. For example, any of the nuclease molecular cargoes described herein can be modified from a native endonuclease sequence and designed to recognize and induce a nick or double-strand break at a target sequence that was not recognized by the native nuclease molecular cargo. Thus, some engineered nucleases have a specificity to induce a nick or double-strand break at a target sequence that is different from the corresponding native nuclease molecular cargo target sequence. Assays for nick or double-strand-break-inducing activity are known and generally measure the overall activity and specificity of the endonuclease on DNA substrates containing the target sequence. The target sequence can be endogenous (or native) to the cell or the target sequence can be exogenous to the cell. A target sequence that is exogenous to the cell is not naturally occurring in the genome of the cell. The target sequence can also exogenous to the polynucleotides of interest that one desires to be positioned at the target locus. In some cases, the target sequence is present only once in the genome of the host cell.

Active variants and fragments of the exemplified target sequences are also provided. Such active variants can comprise at least 65%, 70%, 75%, 80%, 85%, 90%, 91%, 92%, 93%, 94%, 95%, 96%, 97%, 98%, 99% or more sequence identity to the given target sequence, wherein the active variants retain biological activity and hence are capable of being recognized and cleaved by a nuclease molecular cargo in a sequence-specific manner. Assays to measure the double-strand break of a target sequence by a nuclease molecular cargo are known (e.g., TAQMAN® qPCR assay, Frendewey et al. (2010) Methods in Enzymology 476:295-307, herein incorporated by reference in its entirety for all purposes).

The length of the target sequence can vary, and includes, for example, target sequences that are about 30-36 bp for a zinc finger nuclease (ZFN) pair (about 15-18 bp for each ZFN), about 36 bp for a Transcription Activator-Like Effector (TALE) protein or Transcription Activator-Like Effector Nuclease (TALEN), or about 20 bp for a CRISPR/Cas9 guide RNA.

The target sequence of the DNA-binding protein or nuclease molecular cargo can be positioned anywhere in or near the target genomic locus. The target sequence can be located within a coding region of a gene, or within regulatory regions that influence the expression of the gene. A target sequence of the DNA-binding protein or nuclease molecular cargo can be located in an intron, an exon, a promoter, an enhancer, a regulatory region, or any non-protein coding region.

One type of DNA-binding protein that can be employed in the various methods and compositions disclosed herein is a Transcription Activator-Like Effector (TALE). A TALE can be fused or linked to, for example, an epigenetic modification domain, a transcriptional activation domain, or a transcriptional repressor domain. Examples of such domains are described with respect to Cas proteins, below, and can also be found, for example, in WO 2011/145121, herein incorporated by reference in its entirety for all purposes. Correspondingly, one type of nuclease molecular cargo that can be employed in the various methods and compositions disclosed herein is a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease such as Fokl. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See WO 2010/079430; Morbitzer et al. (2010) Proc. Natl. Acad. Sci. U.S.A. 107 (50:21617-21622; Scholze & Boch (2010) Virulence 1:428-432; Christian et al. (2010) Genetics 186:757-761; Li et al. (2011) Nucleic Acids Res. 39(I):359-372; and Miller et al. (2011) Nature Biotechnology 29: 143-148, each of which is herein incorporated by reference in its entirety for all purposes.

The non-specific DNA cleavage domain from the end of the Fokl endonuclease can be used to construct hybrid nucleases that are active in a yeast assay. These remolecular cargoes are also active in plant cells and in animal cells. The Fokl domain functions as a dimer, requiring two constructs with unique DNA binding domains for sites in the target genome with proper orientation and spacing. Both the number of amino acid residues between the TALEN DNA binding domain and the Fokl cleavage domain and the number of bases between the two individual TALEN binding sites are parameters for achieving high levels of activity. The number of amino acid residues between the TALEN DNA binding domain and the Fokl cleavage domain may be modified by introduction of a spacer (distinct from the spacer sequence) between the plurality of TAL effector repeat sequences and the Fokl endonuclease domain. The spacer sequence may be 12 to 30 nucleotides.

The relationship between amino acid sequence and DNA recognition of the TALEN binding domain allows for designable proteins. In this case artificial gene synthesis is problematic because of improper annealing of the repetitive sequence found in the TALE binding domain. One solution to this is to use a publicly available software program (DNAWorks) to calculate oligonucleotides suitable for assembly in a two-step PCR; oligonucleotide assembly followed by whole gene amplification. A number of modular assembly schemes for generating engineered TALE constructs have also been reported. Both methods offer a systematic approach to engineering DNA binding domains that is conceptually similar to the modular assembly method for generating zinc finger DNA recognition domains.

Once the TALEN genes have been assembled, they are inserted into plasmids; the plasmids are then used to transfect the target cell where the gene products are expressed and enter the nucleus to access the genome. TALENs can be used to edit genomes by inducing double-strand breaks (DSB), which cells respond to with repair mechanisms.

Examples of suitable TAL nucleases, and methods for preparing suitable TAL nucleases, are disclosed, e.g., in US 2011/0239315 A1, US 2011/0269234 A1, US 2011/0145940 A1, US 2003/0232410 A1, US 2005/0208489 A1, US 2005/0026157 A1, US 2005/0064474 A1, US 2006/0188987 A1, and US 2006/0063231 A1, each of which is herein incorporated by reference in its entirety for all purposes. In some embodiments, TAL effector nucleases are engineered that cut in or near a target nucleic acid sequence in, for example, a genomic locus of interest, wherein the target nucleic acid sequence is at or near a sequence to be modified.

In some TALENs, each monomer of the TALEN comprises 33-35 TAL repeats that recognize a single base pairvia two hypervariable residues. In some TALENs, the nuclease molecular cargo is a chimeric protein comprising a TAL-repeat-based DNA binding domain operably linked to an independent nuclease such as a Fokl endonuclease. For example, the nuclease molecular cargo can comprise a first TAL-repeat-based DNA binding domain and a second TAL-repeat-based DNA binding domain, wherein each of the first and the second TAL-repeat-based DNA binding domains is operably linked to a Fokl nuclease, wherein the first and the second TAL-repeat-based DNA binding domain recognize two contiguous target DNA sequences in each strand of the target DNA sequence separated by a spacer sequence of varying length (12-20 bp), and wherein the Fokl nuclease subunits dimerize to create an active nuclease that makes a double strand break at a target sequence.

Transcription Activator-Like Effector Nucleases (TALENs) are artificial restriction enzymes generated by fusing the TAL effector DNA binding domain to a DNA cleavage domain. These remolecular cargoes enable efficient, programmable, and specific DNA cleavage and represent powerful tools for genome editing in situ. Transcription activator-like effectors (TALEs) can be quickly engineered to bind practically any DNA sequence. The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See U.S. Pat. Nos. 8,586,363; 8,450,471; 8,440,431; 8,440,432; and 8,697,853, all of which are incorporated by reference herein in their entirety.

Another example of a DNA-binding protein is a zinc finger protein. Such zinc finger proteins can be linked or fused to, for example, an epigenetic modification domain, a transcriptional activation domain, or a transcriptional repressor domain. Examples of such domains are described with respect to Cas proteins, below, and can also be found, for example, in WO 2011/145121, herein incorporated by reference in its entirety for all purposes. Correspondingly, another example of a nuclease molecular cargo that can be employed in the various methods and compositions disclosed herein is a zinc-finger nuclease (ZFN). In some ZFNs, each monomer of the ZFN comprises three or more zinc finger-based DNA binding domains, wherein each zinc finger-based DNA binding domain binds to a 3 bp subsite. In other ZFNs, the ZFN is a chimeric protein comprising a zinc finger-based DNA binding domain operably linked to an independent nuclease such as a Fokl endonuclease. For example, the nuclease molecular cargo can comprise a first ZFN and a second ZFN, wherein each of the first ZFN and the second ZFN is operably linked to a Fokl nuclease subunit, wherein the first and the second ZFN recognize two contiguous target DNA sequences in each strand of the target DNA sequence separated by about 5-7 bp spacer, and wherein the Fokl nuclease subunits dimerize to create an active nuclease that makes a double strand break. See, e.g., US 2006/0246567; US 2008/0182332; US 2002/0081614; US 2003/0021776; WO 2002/057308 A2; US 2013/0123484; US 2010/0291048; WO 2011/017293 A2; and Gaj et al. (2013) Trends in Biotechnology 31(7):397-405, each of which is herein incorporated by reference in its entirety for all purposes.

### Conjugation of Molecular Cargo to Antigen-Binding Protein

In some embodiments, a molecular cargo described herein, e.g., a polynucleotide molecule described herein, or a liposome or LNP, is conjugated to an anti-TfR antigen-binding protein for delivery to a site of interest (e.g., brain or muscle). In some embodiments, the anti-TfR antigen-binding protein is conjugated to at least one molecular cargo (e.g., polynucleotide molecule, or liposome or LNP).

In some embodiments, an anti-TfR antigen-binding protein is conjugated to the 5′ terminus of a polynucleotide molecule, the 3′ terminus of a polynucleotide molecule, an internal site on a polynucleotide molecule, or in any combinations thereof.

In some embodiments, the anti-TfR antigen-binding protein is conjugated to at least one molecular cargo (e.g., at least one polynucleotide molecule and/or liposome or LNP). In some embodiments, the anti-TfR antigen-binding protein is conjugated to at least 2, 3, 4, 5, 6, 7, 8, 10, 12, 16, 20, 24, 30 or more molecular cargoes described herein (e.g., at least 2, 3, 4, 5, 6, 7, 8, 10, 12, 16, 20, 24, 30 or more polynucleotide molecules, and/or liposomes or LNPs).

In some embodiments, a protein-drug conjugate described herein comprises an anti-TfR antibody conjugated to one siRNA molecule. In some embodiments, a protein-drug conjugate described herein comprises an anti-TfR antibody conjugated to two siRNA molecules.

In some embodiments, a protein-drug conjugate described herein comprises an anti-TfR scFv conjugated to one siRNA molecule. In some embodiments, a protein-drug conjugate described herein comprises an anti-TfR scFv conjugated to two siRNA molecules.

In some embodiments, a protein-drug conjugate described herein comprises an anti-TfR Fab conjugated to one siRNA molecule. In some embodiments, a protein-drug conjugate described herein comprises an anti-TfR Fab conjugated to two siRNA molecules.

In some embodiments, a protein-drug conjugate described herein comprises an anti-TfR one-armed antibody conjugated to one siRNA molecule. In some embodiments, a protein-drug conjugate described herein comprises an anti-TfR one-armed antibody conjugated to two siRNA molecules.

In some embodiments, the anti-TfR antigen-binding protein is conjugated to a molecular cargo (e.g., polynucleotide molecule, or liposome or LNP) non-specifically. In some embodiments, the anti-TfR antigen-binding protein is conjugated to a molecular cargo (e.g., polynucleotide molecule, or liposome or LNP) via a lysine residue or a cysteine residue, in a non-site-specific manner. In some embodiments, the anti-TfR antigen-binding protein is conjugated to a molecular cargo (e.g., polynucleotide molecule, or liposome or LNP) via a lysine residue (e.g., lysine residue present in the anti-TfR antigen-binding protein) in a non-site specific manner. In some cases, the anti-TfR antigen-binding protein is conjugated to a molecular cargo (e.g., polynucleotide molecule, or liposome or LNP) via a cysteine residue (e.g., cysteine residue present in the anti-TfR antigen-binding protein) in a non-site-specific manner.

In some embodiments, the anti-TfR antigen-binding protein is conjugated to a molecular cargo (e.g., polynucleotide molecule, or liposome or LNP) in a site-specific manner. In some embodiments, the anti-TfR antigen-binding protein is conjugated to a molecular cargo (e.g., polynucleotide molecule, or liposome or LNP) through a lysine residue, a cysteine residue, at the N-terminus, at the C-terminus, an unnatural amino acid, or an enzyme-modified or enzyme-catalyzed residue, via a site-specific manner. In some embodiments, the anti-TfR antigen-binding protein is conjugated to a molecular cargo (e.g., polynucleotide molecule, or liposome or LNP) through a lysine residue (e.g., lysine residue present in the anti-TfR antigen-binding protein) via a site-specific manner. In some embodiments, the anti-TfR antigen-binding protein is conjugated to a molecular cargo (e.g., polynucleotide molecule, or liposome or LNP) through a cysteine residue (e.g., cysteine residue present in the anti-TfR antigen-binding protein) via a site-specific manner. In some embodiments, the anti-TfR antigen-binding protein is conjugated to a molecular cargo (e.g., polynucleotide molecule, or liposome or LNP) at the N-terminus via a site-specific manner. In some embodiments, the anti-TfR antigen-binding protein is conjugated to a molecular cargo (e.g., polynucleotide molecule, or liposome or LNP) at the C-terminus via a site-specific manner. In some embodiments, the anti-TfR antigen-binding protein is conjugated to a molecular cargo (e.g., polynucleotide molecule, or liposome or LNP) through an unnatural amino acid via a site-specific manner. In some embodiments, the anti-TfR antigen-binding protein is conjugated to a molecular cargo (e.g., polynucleotide molecule, or liposome or LNP) through an enzyme-modified or enzyme-catalyzed residue via a site-specific manner.

In some embodiments, one or more molecular cargoes (e.g., polynucleotide molecule, and/or liposome or LNP) is conjugated to an anti-TfR antigen-binding protein. In some embodiments, about 1, 2, 3, 4, 5, 6, 7, 8, 9, 10, 11, 12, 13, 14, 15, 16, 20, 24, 30, 36 or more molecular cargoes (e.g., polynucleotide molecule, and/or liposome or LNP) are conjugated to one anti-TfR antigen-binding protein. In some embodiments, 1 molecular cargo is conjugated to one anti-TfR antigen-binding protein. In some embodiments, 2 molecular cargoes are conjugated to one anti-TfR antigen-binding protein. In some embodiments, 3 molecular cargoes are conjugated to one anti-TfR antigen-binding protein. In some embodiments, 4 molecular cargoes are conjugated to one anti-TfR antigen-binding protein. In some embodiments, 5 molecular cargoes are conjugated to one anti-TfR antigen-binding protein. In some embodiments, 6 molecular cargoes are conjugated to one anti-TfR antigen-binding protein. In some embodiments, 7 molecular cargoes are conjugated to one anti-TfR antigen-binding protein. In some embodiments, 8 molecular cargoes are conjugated to one anti-TfR antigen-binding protein. In some embodiments, 9 molecular cargoes are conjugated to one anti-TfR antigen-binding protein. In some embodiments, 10 molecular cargoes are conjugated to one anti-TfR antigen-binding protein. In some embodiments, 11 molecular cargoes are conjugated to one anti-TfR antigen-binding protein. In some embodiments, 12 molecular cargoes are conjugated to one anti-TfR antigen-binding protein. In some embodiments, 13 molecular cargoes are conjugated to one anti-TfR antigen-binding protein. In some embodiments, 14 molecular cargoes are conjugated to one anti-TfR antigen-binding protein. In some embodiments, 15 molecular cargoes are conjugated to one anti-TfR antigen-binding protein. In some embodiments, 16 molecular cargoes are conjugated to one anti-TfR antigen-binding protein. In some cases, the one or more molecular cargoes are the same. In other cases, the one or more molecular cargoes are different.

In some embodiments, the number of molecular cargoes conjugated to an anti-TfR antigen-binding protein forms a ratio. In some embodiments, the ratio is referred to as a DAR (drug-to-antibody) ratio, in which the drug as referred to herein is a molecular cargo described herein (e.g., polynucleotide molecule, or liposome or LNP). In some embodiments, the DAR ratio of the molecular cargo to anti-TfR antigen-binding protein is about 1, 2, 3, 4, 5, 6, 7, 8, 9, 10, 11, 12, 13, 14, 15, 16, 20, 24, 30, 36 or greater. In some embodiments, the DAR ratio of the molecular cargo to anti-TfR antigen-binding protein is about 1 or greater. In some embodiments, the DAR ratio of the molecular cargo to anti-TfR antigen-binding protein is about 2 or greater. In some embodiments, the DAR ratio of the molecular cargo to anti-TfR antigen-binding protein is about 3 or greater. In some embodiments, the DAR ratio of the molecular cargo to anti-TfR antigen-binding protein is about 4 or greater. In some embodiments, the DAR ratio of the molecular cargo to anti-TfR antigen-binding protein is about 5 or greater. In some embodiments, the DAR ratio of the molecular cargo to anti-TfR antigen-binding protein is about 6 or greater. In some embodiments, the DAR ratio of the molecular cargo to anti-TfR antigen-binding protein is about 7 or greater. In some embodiments, the DAR ratio of the molecular cargo to anti-TfR antigen-binding protein is about 8 or greater. In some embodiments, the DAR ratio of the molecular cargo to anti-TfR antigen-binding protein is about 9 or greater. In some embodiments, the DAR ratio of the molecular cargo to anti-TfR antigen-binding protein is about 10 or greater. In some embodiments, the DAR ratio of the molecular cargo to anti-TfR antigen-binding protein is about 11 or greater. In some embodiments, the DAR ratio of the molecular cargo to anti-TfR antigen-binding protein is about 12 or greater. In some embodiments, the DAR ratio of the molecular cargo to anti-TfR antigen-binding protein is about 16 or greater. In some embodiments, the DAR ratio of the molecular cargo to anti-TfR antigen-binding protein is about 20 or greater. In some embodiments, the DAR ratio of the molecular cargo to anti-TfR antigen-binding protein is about 24 or greater.

In some embodiments, the DAR ratio of the molecular cargo to anti-TfR antigen-binding protein is about 1, 2, 3, 4, 5, 6, 7, 8, 9, 10, 11, 12, 13, 14, 15, 16, 20, 24, 30, or 36. In some embodiments, the DAR ratio of the molecular cargo to anti-TfR antigen-binding protein is about 1. In some embodiments, the DAR ratio of the molecular cargo to anti-TfR antigen-binding protein is about 2. In some embodiments, the DAR ratio of the molecular cargo to anti-TfR antigen-binding protein is about 3. In some embodiments, the DAR ratio of the molecular cargo to anti-TfR antigen-binding protein is about 4. In some embodiments, the DAR ratio of the molecular cargo to anti-TfR antigen-binding protein is about 5. In some embodiments, the DAR ratio of the molecular cargo to anti-TfR antigen-binding protein is about 6. In some embodiments, the DAR ratio of the molecular cargo to anti-TfR antigen-binding protein is about 7. In some embodiments, the DAR ratio of the molecular cargo to anti-TfR antigen-binding protein is about 8. In some embodiments, the DAR ratio of the molecular cargo to anti-TfR antigen-binding protein is about 9. In some embodiments, the DAR ratio of the molecular cargo to anti-TfR antigen-binding protein is about 10. In some embodiments, the DAR ratio of the molecular cargo to anti-TfR antigen-binding protein is about 11. In some embodiments, the DAR ratio of the molecular cargo to anti-TfR antigen-binding protein is about 12. In some embodiments, the DAR ratio of the molecular cargo to anti-TfR antigen-binding protein is about 13. In some embodiments, the DAR ratio of the molecular cargo to anti-TfR antigen-binding protein is about 14. In some embodiments, the DAR ratio of the molecular cargo to anti-TfR antigen-binding protein is about 15. In some embodiments, the DAR ratio of the molecular cargo to anti-TfR antigen-binding protein is about 16.

In some embodiments, liposome or LNP functionalization with binding moieties is carried out via the adsorption phenomenon, covalent-nature binding, or binding by the use of adapter molecules or linkers.

### Adsorption

This phenomenon is a non-covalent immobilization strategy that comprises physical adsorption and ionic binding. Physical adsorption occurs via weak interactions such as hydrogen bonding, electrostatic, hydrophobic and Van der Waals attractive forces, while ionic binding occurs between the opposite charges of the anti-TfR antigen-binding protein and liposome or LNP surfaces. However, when compared to other methodologies such as covalent binding, adsorption provides less stability. On the other hand, the fact that the interaction is non-covalent may allow easier release of the cargo in the tumor tissue.

### Covalent Strategies

Covalent binding requires prior activation of the LNPs. In some embodiments, covalent strategies occur via carbodiimide chemistry, maleimide chemistry or “click chemistry”, as discussed in detail below.

### Conjugation Chemistry

In some embodiments, a molecular cargo described herein (e.g., a polynucleotide molecule described herein, or a liposome or LNP) is conjugated to an anti-TfR antigen-binding protein. In some embodiments, a molecular cargo described herein (e.g., a polynucleotide molecule described herein, or a liposome or LNP) is conjugated to an anti-TfR antigen-binding protein directly. In some embodiments, a molecular cargo described herein (e.g., a polynucleotide molecule described herein, or a liposome or LNP) is conjugated to an anti-TfR antigen-binding protein via a linker covalently connecting the anti-TfR antigen-binding protein with the molecular cargo. In some embodiments, the anti-TfR antigen-binding protein is an antibody or antigen binding fragment thereof (e.g., scFv, Fab, or one-armed antibody).

In some embodiments, the molecular cargo described herein (e.g., a polynucleotide molecule described herein, or a liposome or LNP) is conjugated to the anti-TfR antigen-binding protein by a chemical ligation process. In some embodiments, the molecular cargo described herein (e.g., a polynucleotide molecule described herein, or a liposome or LNP) is conjugated to the anti-TfR antigen-binding protein by a native ligation. In some embodiments, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some embodiments, the conjugation is as described in U.S. Pat. No. 8,936,910. In some embodiments, the molecular cargo described herein (e.g., a polynucleotide molecule described herein, or a liposome or LNP) is conjugated to the anti-TfR antigen-binding protein either site-specifically or non-specifically via native ligation chemistry.

In some embodiments, the molecular cargo described herein (e.g., a polynucleotide molecule described herein, or a liposome or LNP) is conjugated to the anti-TfR antigen-binding protein by a site-directed method utilizing a “traceless” coupling technology (Philochem). In some embodiments, the “traceless” coupling technology utilizes an N-terminal 1,2-aminothiol group on the anti-TfR antigen-binding protein which is then conjugated with a molecular cargo described herein (e.g., a polynucleotide molecule described herein, or a liposome or LNP) containing an aldehyde group. (see Casi et al., “Site-specific traceless coupling of potent cytotoxic drugs to recombinant antibodies for pharmacodelivery,” JACS 134(13): 5887-5892 (2012)).

In some embodiments, the molecular cargo described herein (e.g., a polynucleotide molecule described herein, or a liposome or LNP) is conjugated to the anti-TfR antigen-binding protein by a site-directed method utilizing an unnatural amino acid incorporated into the anti-TfR antigen-binding protein. In some embodiments, the unnatural amino acid comprises p-acetylphenylalanine (pAcPhe). In some embodiments, the keto group of pAcPhe is selectively coupled to an alkoxy-amine derivatived conjugating moiety to form an oxime bond. (see Axup et al., “Synthesis of site-specific antibody-drug conjugates using unnatural amino acids, “PNAS 109(40): 16101-16106 (2012)).

In some embodiments, the molecular cargo described herein (e.g., a polynucleotide molecule described herein, or a liposome or LNP) is conjugated to the anti-TfR antigen-binding protein by a site-directed method utilizing an enzyme-catalyzed process. In some embodiments, the site-directed method utilizes SMARTag™ technology (Catalent, Inc.). In some embodiments, the SMARTag™ technology comprises generation of a formylglycine (FGIy) residue from cysteine by formylglycine-generating enzyme (FGE) through an oxidation process under the presence of an aldehyde tag and the subsequent conjugation of FGIy to an alkylhydraine-functionalized molecular cargo described herein (e.g., a polynucleotide molecule described herein, or a liposome or LNP) via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013))

In some embodiments, the enzyme-catalyzed process comprises transglutaminase (TG), e.g., microbial transglutaminase (mTG). In some cases, the molecular cargo described herein (e.g., a polynucleotide molecule described herein, or a liposome or LNP) is conjugated to the anti-TfR antigen-binding protein utilizing a microbial transglutaminase-catalyzed process. In some embodiments, mTG catalyzes the formation of a covalent bond between the amide side chain of a glutamine within the recognition sequence and a primary amine of a functionalized molecular cargo described herein (e.g., a polynucleotide molecule described herein, or a liposome or LNP). In some embodiments, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some embodiments, a sequence of amino acids comprising an acceptor glutamine residue are incorporated into (e.g., appended to) a polypeptide sequence, under suitable conditions, for recognition by a TG. This sequence leads to cross-linking by the TG through a reaction between an amino acid side chain within the sequence of amino acids and a reaction partner. The recognition tag may be a peptide sequence that is not naturally present in the polypeptide comprising the TG recognition tag. In some embodiments, the TG recognition tag comprises at least one Gln. In some embodiments, the TGase recognition tag comprises an amino acid sequence XXQX (SEQ ID NO: 438), wherein X is any amino acid (e.g., conventional amino acid Leu, Ala, Gly, Ser, Val, Phe, Tyr, His, Arg, Asn, Glu, Asp, Cys, Gln, Ile, Met, Pro, Thr, Lys, or Trp or nonconventional amino acid). In some embodiments, the acyl donor glutamine-containing tag comprises an amino acid sequence selected from the group consisting of LLQGG (SEQ ID NO: 439), LLQG (SEQ ID NO: 440), LSLSQG (SEQ ID NO: 441), gGGLLQGG (SEQ ID NO: 442), gLLQG (SEQ ID NO: 443), LLQ (SEQ ID NO: 444), gSPLAQSHGG (SEQ ID NO: 445), gLLQGGG (SEQ ID NO: 446), gLLQGG (SEQ ID NO: 447), gLLQ (SEQ ID NO: 448), LLQLLQGA (SEQ ID NO: 449), LLQGA (SEQ ID NO: 450), LLQYQGA (SEQ ID NO: 451), LLQGSG (SEQ ID NO: 452), LLQYQG (SEQ ID NO: 453), LLQLLQG (SEQ ID NO: 454), SLLQG (SEQ ID NO: 455), LLQLQ (SEQ ID NO: 456), LLQLLQ (SEQ ID NO: 457), and LLQGR (SEQ ID NO: 458). See, e.g., PCT Publication No. WO2012/059882. In some embodiments, the acyl donor glutamine-containing tag is present at the N-terminus of the antigen-binding protein. In some embodiments, the acyl donor glutamine-containing tag is present at the C-terminus of the antigen-binding protein. In some embodiments, the acyl donor glutamine-containing tag is present both at the N-terminus and the C-terminus of the antigen-binding protein.

In some embodiments, the molecular cargo described herein (e.g., a polynucleotide molecule described herein, or a liposome or LNP) is conjugated to the anti-TfR antigen-binding protein by a method as described in PCT Publication No. WO2014/140317, which utilizes a sequence-specific transpeptidase. In some embodiments, the molecular cargo described herein (e.g., a polynucleotide molecule described herein, or a liposome or LNP) is conjugated to the anti-TfR antigen-binding protein by a method as described in U.S. Patent Publication Nos. 2015/0105539 and 2015/0105540.

In some embodiments, the molecular cargo described herein (e.g., a polynucleotide molecule described herein, or a liposome or LNP) is conjugated to the anti-TfR antigen-binding protein utilizing Azide-Alkyne Cycloaddition (CuAAC) click chemistry. Azides and alkynes can undergo catalyst free [3+2]cycloaddition by a using the reaction of activated alkynes with azides. Such catalyst-free [3+2]cycloaddition can be used in the methods described herein to conjugate an anti-TfR antigen-binding protein and the molecular cargo described herein (e.g., a polynucleotide molecule described herein, or a liposome or LNP). Alkynes can be activated by ring strain such as, by way of example only, eight-membered ring structures, or nine-membered, appending electron-withdrawing groups to such alkyne rings, or alkynes can be activated by the addition of a Lewis acid such as, by way of example only, Au(I) or Au(III).

Alkynes activated by ring strain have been described and used in “copperless” [3+2]cycloaddition. For example, the cyclooctynes and difluorocyclooctynes described by Agard et al., J. Am. Chem. Soc., 126 (46):15046-15047 (2004), the dibenzocyclooctynes described by Boons et al., PCT International Publication No. WO 2009/067663 A1 (2009), the aza-dibenzocyclooctynes described by Debets et al., Chem. Comm., 46:97-99 (2010), and the cyclononynes described by Dommerholt et al., Angew. Chem. 122:9612-9615 (2010)). In some embodiments, a tetrazine (Tzn)-activated anti-TfR antigen-binding protein may be cross-linked to a trans-cyclooctene (TCO)-activated molecular cargo described herein (e.g., a polynucleotide molecule described herein, or a liposome or LNP). In some embodiments, a TCO-activated anti-TfR antigen-binding protein may be crosslinked to a Tzn-activated molecular cargo described herein (e.g., a polynucleotide molecule described herein, or a liposome or LNP).

### Linkers

Complexes described herein generally comprise a linker that connects a binding agent to a molecular cargo (e.g., a polynucleotide molecule, a liposome or an LNP). A linker comprises at least one covalent bond. In some embodiments, a linker may be a single bond, e.g., a disulfide bond or disulfide bridge, that connects a binding agent to a polynucleotide molecule, or a liposome or LNP. However, in some embodiments, a linker may connect a binding agent to a polynucleotide molecule, or a liposome or LNP through multiple covalent bonds. A linker is generally stable in vitro and in vivo, and may be stable in certain cellular environments. Additionally, generally a linker does not negatively impact the functional properties of either the binding agent or the polynucleotide molecule, or a liposome or LNP. Examples and methods of synthesis of linkers are known in the art (see, e.g. Kline, T. et al. “Methods to Make Homogenous Antibody Drug Conjugates.” Pharmaceutical Research, 2015, 32:11, 3480-3493; Jain, N. et al. “Current ADC Linker Chemistry” Pharm Res. 2015, 32:11, 3526-3540; McCombs, J. R. and Owen, S. C., “Antibody Drug Conjugates: Design and Selection of Linker, Payload and Conjugation Chemistry” AAPS J. 2015, 17:2, 339-351).

A precursor to a linker typically will contain two different reactive species that allow for attachment to both the binding agent and a polynucleotide molecule, or a liposome or LNP. In some embodiments, the two different reactive species may be a nucleophile and/or an electrophile. In some embodiments, a linker is connected to a binding agent via conjugation to a lysine residue or a cysteine residue of the binding agent. In some embodiments, a linker is connected to a cysteine residue of a muscle-targeting agent via a maleimide-containing linker, wherein optionally the maleimide-containing linker comprises a maleimidocaproyl or maleimidomethyl cyclohexane-1-carboxylate group. In some embodiments, a linker is connected to a cysteine residue of a muscle-targeting agent or thiol functionalized molecular cargo via a 3-arylpropionitrile functional group. In some embodiments, a linker is connected to a binding agent and/or a polynucleotide molecule or an LNP via an amide bond, a hydrazide, a triazole, a thioether or a disulfide bond.

In some embodiments, a linker described herein is a cleavable linker or a non-cleavable linker. In some embodiments, the linker is a cleavable linker. In other embodiments, the linker is a non-cleavable linker.

### Cleavable Linkers

A cleavable linker may be a protease-sensitive linker, a pH-sensitive linker, or a glutathione-sensitive linker. These linkers are generally cleavable only intracellularly and are preferably stable in extracellular environments.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include 3-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline dipeptide sequence. In some embodiments, a protease-sensitive linker can be cleaved by a lysosomal protease, e.g. cathepsin B, and/or an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by a disulfide exchange reaction with a glutathione species inside a cell. In some embodiments, the disulfide moiety further comprises at least one amino acid, e.g. a cysteine residue.

### Non-Cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyneazide, a peptide sequence comprising a LPXT sequence, a thioether, a biotin, a biphenyl, repeating units of polyethylene glycol or equivalent compounds, acid esters, acid amides, sulfamides, and/or an alkoxy-amine linker. In some embodiments, sortase-mediated ligation will be utilized to covalently link a muscle-targeting agent comprising a LPXT sequence to a molecular cargo comprising a (G), sequence (see, e.g. Proft T. Sortase-mediated protein ligation: an emerging biotechnology tool for protein modification and immobilization. Biotechnol Lett. 2010, 32(1):1-10).

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S; an optionally substituted heterocyclylene further comprising at least one heteroatom selected from N, O, and S; an imino, an optionally substituted nitrogen species, an optionally substituted oxygen species, an optionally substituted sulfur species, or a poly(alkylene oxide), e.g. polyethylene oxide or polypropylene oxide.

In some cases, the linker is a non-polymeric linker. A non-polymeric linker refers to a linker that does not contain a repeating unit of monomers generated by a polymerization process. Exemplary non-polymeric linkers include, but are not limited to, C1-C30 alkyl group (e.g., a C5, C4, C 3, C 2, or C1 alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C1-C30 alkyl group (e.g., a C5, C4, C 3, C 2, or C1 alkyl group), a homobifunctional cross linker, a heterobifunctional cross linker, a peptide linker, a traceless linker, a self-immolative linker, a maleimide-based linker, or a combination thereof. In additional cases, the non-polymeric linker does not comprise more than two of the same type of linkers, e.g., more than two homobifunctional cross linkers, or more than two peptide linkers. In further cases, the non-polymeric linker optionally comprises one or more reactive functional groups. In one embodiment, the linker has a structure

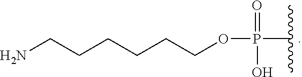 [SMILES: *P(=O)(O)OCCCCCCN]

In some cases, the non-polymeric linker does not encompass a polyalkylene oxide (e.g., PEG). In some cases, the non-polymeric linker does not encompass a PEG. In some embodiments, the linker comprises a homobifunctional linker. Exemplary homobifunctional linkers include, but are not limited to, organoazide, organoalkyne, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido) butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-113-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, a,a′-p-diaminodiphenyl, diiodo-p-xylene sulfonic acid, N,N′-ethylene-bis(iodoacetamide), or N,N′-hexamethylene-bis(iodoacetamide). In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio) propionate (sPDP), long-chain N-succinimidyl 3-(2-pyridyldithio) propionate (LC-sPDP), water-soluble-long-chain N-succinimidyl 3-(2-pyridyldithio) propionate (sulfo-LCsPDP), succinimidyloxycarbonyl-a-methyl-a-(2-pyridyldithio) toluene (sMPT), sulfosuccinimidy1-6-[a-methyl-a-(2-pyridyldithio)toluamido]hexanoate (sulfo-LC-sMPT), succinimidyl-4-(N-maleimidomethyl) cyclohexane-1-car-boxylate (sMCC), sulfosuccinimidyl-4-(N-maleimidomethyl) cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MB s), N-succinimidyl (4-iodoacteyl)aminobenzoate (slAB), sulfosuccinimidyl (4-iodoacteyl)aminobenzoate (sulfo-slAB), succinimidyl-4-(p-maleimidophenyl)butyrate (sMPB), sulfosuccinimidyl-4-(p-maleimidophenyl)butyrate (sulfo-sMPB), N-(y-maleimidobutyryloxy)succinimide ester (GMBs), N-(y-maleimidobutyryloxy)sulfosuccinimide ester (sulfo-GMBs), succinimidyl 6-((iodoacetyl)amino)hexanoate (slAX), succinimidyl 6-[6-(((iodoacetyl)amino) hexanoyl)amino]hexanoate (slAXX), succinimidyl 4-(((iodoacetyl) amino)methyl)cyclohexane-I-carboxylate (slAC), succinimidyl 6-((((4-iodoacetyl)amino)methyl)cyclohexane-1-carbonyl)amino) hexanoate (slACX), p-nitrophenyl iodoacetate (NPIA), carbonyl-reactive and sulfhydrylreactive cross-linkers such as 4-(4-N-maleimidophenyl) butyric acid hydrazide (MPBH), 4-(N-maleimidomethyl) cyclohexane-I-carboxyl-hydrazide-8 (M2C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido2′-nitrophenylamino)hexanoate (sANPAH), sulfo succinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidopheny1)1,3′-dithiopropionate (sADP), N-sulfosuccinimidyl(4-azidophenyl)-1,3′-dithiopropionate (sulfo-sADP), sulfosuccinimidyl 4-(p-azidophenyl) butyrate (sulfo-sAPB), sulfosuccinimidyl 2-(7-azido-4-methylcoumarin-3-acetamide)ethy1-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive crosslinkers such as 1-(p-Azidosalicylamido)-4-(iodoacetamido) butane (AsIB), N-[4-(p-azidosalicylamido)buty1]-3′-(2′-pyridyldithio)propionamide (APDP), benzophenone-4-iodoacetamide, benzophenone-4-maleimide carbonylreactive and photoreactive cross-linkers such as p-azidobenzoyl hydrazide (ABH), carboxylate-reactive and photoreactive cross-linkers such as 4-(p-azidosalicylamido) butylamine (AsBA), and arginine-reactive and photoreactive cross-linkers such as p-azidophenyl glyoxal (APG).

In some embodiments, the linker comprises a reactive functional group. In some cases, the reactive functional group comprises a nucleophilic group that is reactive to an electrophilic group present on an anti-TfR antigen-binding protein. Exemplary electrophilic groups include carbonyl groups such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide.

In some embodiments, the linker comprises a maleimide group. In some embodiments, the maleimide group is also referred to as a maleimide spacer. In some embodiments, the maleimide group further encompasses a caproic acid, forming maleimidocaproyl (mc). In some cases, the linker comprises maleimidocaproyl (mc). In some cases, the linker is maleimidocaproyl (mc). In other embodiments, the maleimide group comprises a maleimidomethyl group, such as succinimidy1-4-(N-maleimidomethyl)cyclohexane-I-carboxylate (sMCC) or sulfosuccinimidy1-4-(N-maleimidomethyl)cyclohexane-1-carboxylate (sulfo-sMCC) described above.

In some embodiments, the maleimide group is a self-stabilizing maleimide. In some embodiments, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some embodiments, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some embodiments, the linker comprises a self-stabilizing maleimide. In some embodiments, the linker is a self-stabilizing maleimide.

In some embodiments, the linker comprises at least one azide moiety, e.g., as part of an organoazide moiety. In some embodiments, the linker comprises at least one alkyne moiety, e.g., as part of an organoalkyne moiety. In one embodiment, the alkyne is an activated alkyne. In some embodiments, the linker comprises a trizole (e.g., formed via a 1,3-cycloaddition reaction of an azide and an alkyne). In some embodiments, the linker comprises a Diels-Alder adduct.

In some embodiments, the linker comprises a peptide moiety. In some embodiments, the peptide moiety comprises at least 2, 3, 4, 5, or 6 more amino acid residues. In some embodiments, the peptide moiety comprises at most 2, 3, 4, 5, 6, 7, or 8 amino acid residues. In some embodiments, the peptide moiety comprises about 2, about 3, about 4, about 5, or about 6 amino acid residues. In some embodiments, the peptide moiety is a cleavable peptide moiety (e.g., either enzymatically or chemically). In some embodiments, the peptide moiety is a non-cleavable peptide moiety. In some embodiments, the peptide moiety comprises Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some embodiments, the linker comprises a peptide moiety such as: Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some cases, the linker comprises Val-Cit. In some cases, the linker is Val-Cit.

In some embodiments, the linker comprises a benzoic acid group, or its derivatives thereof. In some embodiments, the benzoic acid group or its derivatives thereof comprise paraaminobenzoic acid (PABA). In some embodiments, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some embodiments, the maleimide group is maleimidocaproyl (mc). In some embodiments, the peptide group is val-cit. In some embodiments, the benzoic acid group is PABA. In some embodiments, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some embodiments, the linker comprises a linker described in U.S. Pat. No. 9,089,614 or PCT Publication No. WO 2015/038426.

In some embodiments, the linker is a dendritic type linker. In some embodiments, the dendritic type linker comprises a branching, multifunctional linker moiety. In some embodiments, the dendritic type linker is used to increase the molar ratio of polynucleotide B to the anti-TfR antigen-binding protein. In some embodiments, the dendritic type linker comprises PAMAM dendrimers. In some embodiments, the dendritic type linker comprises triazoles. In some embodiments, the triazoles are connected by PEG links. In some embodiments, the linkers are as described in WO 2022/015656.

In some embodiments, the linker is a traceless linker or a linker in which after cleavage does not leave behind a linker moiety (e.g., an atom or a linker group) to an anti-TfR antigen-binding protein or a polynucleotide B. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some embodiments, the linker is a traceless linker described in Blaney, et al., “Traceless solid-phase organic synthesis,” Chem. Rev. 102: 2607-2024 (2002). In some embodiments, a linker is a traceless linker as described in U.S. Pat. No. 6,821,783.

In some embodiments, the linker is a linker described in U.S. Pat. Nos. 6,884,869; 7,498,298; 8,288,352; 8,609,105; or 8,697,688; U.S. Patent Publication Nos. US2014/0127239; US2013/028919; US2014/286970; US2013/0309256; US2015/037360; and US2014/0294851; or International Application Publication Nos. WO2015/057699; WO2014/080251; WO2014/197854; WO2014/145090; WO2014/177042, WO2022/015656.

In some embodiments, a linker is a bond, i.e., a linker is absent. In some cases, a linker is a non-polymeric linker. In some cases, a linker is a polymeric linker.

In some embodiments, the linker comprises an alkyl group. In some embodiments, the linker comprises a C1-C30 alkyl group, or a C1-C24 alkyl group, or a C1-C20 alkyl group, or a C1-C16 alkyl group, or a C1-C12 alkyl group, or a C1-C10 alkyl group, or a C1-C8 alkyl group, or a C1-C6 alkyl group, or a C1-C4 alkyl group. In some cases, a linker is a C1-C6 alkyl group, such as for example, a C 3, C 4, C 3, C 2, or C1 alkyl group. In some cases, the C1-C6 alkyl group is an unsubstituted C1-C6 alkyl group. As used in the context of a linker, alkyl means a saturated straight or branched hydrocarbon radical containing up to six carbon atoms. In some embodiments, the linker comprises a homobifunctional linker or a heterobifunctional linker described supra.

In some cases, a linker is an oligomeric or a polymeric linker. In some embodiments, a linker is a natural or synthetic oligomer or polymer, consisting of branched or unbranched monomers, and/or cross-linked network of monomers in two or three dimensions. In some embodiments, the linker comprises a polysaccharide, lignin, rubber, or polyalkylen oxide (e.g., polyethylene glycol).

In some embodiments, the at least one polymeric linker includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylene terephthalate (also known as poly(ethylene terephthalate), PET, PETG, or PETE), polytetramethylene glycol (PTG), or polyurethane as well as mixtures thereof. In some embodiments, the linker comprises polyalkylene oxide. In some embodiments, the linker comprises PEG. In some embodiments, the linker comprises polyethylene imide (PEI) or hydroxy ethyl starch (HES).

In some embodiments, the linker comprises a polyalkylene oxide (e.g., PEG) comprising discrete ethylene oxide units. In some cases, the linker comprises between about 2 and about 48 ethylene oxide units. In some cases, the polymer moiety C comprises about 2, about 3, about 4, about 5, about 6, about 7, about 8, about 9, about 10, about 11, about 12, about 13, about 14, about 15, about 16, about 17, about 18, about 19, about 20, about 24, about 30, about 36, about 42, or about 48 ethylene oxide units.

In some embodiments, the anti-TfR antigen-binding protein is conjugated to the molecular cargo described herein (e.g., a polynucleotide molecule described herein, or a liposome or LNP) using a protamine linker, as disclosed in the U.S. Patent Application Publication Nos. US2002/0132990, US2004/0023902, US2007/012152, and US2010/0209440. In some embodiments, a protamine linker encompassed for use in the protein-drug conjugates described herein comprises a sequence disclosed in US 2010/0209440.

Acid cleavable linkers can also be used in the protein-drug conjugates described herein and include, but are not limited to, bismaleimideothoxy propane, adipic acid dihydrazide linkers (see, e.g., Fattom et al., Infection & Immun. 60:584 589, 1992) and acid labile transferrin conjugates that contain a sufficient portion of transferrin to permit entry into the intracellular transferrin cycling pathway (see, e.g., Welhoner et al., J. Biol. Chem. 266:4309 4314, 1991). Conjugates linked via acid cleavable linkers should be preferentially cleaved in acidic intracellular compartments, such as the endosome.

Photocleavable linkers can also be used with in the protein-drug conjugates described herein. Photocleavable linkers are cleaved upon exposure to light (see, e.g., Goldmacher et al., Bioconj. Chem. 3:104-107, 1992), thereby releasing the targeted agent upon exposure to light. (Hazum et al., Proc. Eur. Pept. Symp., 16th, Brunfeldt, K (Ed), pp. 105 110, 1981; nitrobenzyl group as a photocleavable protective group for cysteine; Yen et al., Makromol. Chem 190:69 82, 1989; water soluble photocleavable copolymers, including hydroxypropylmethacrylamide copolymer, glycine copolymer, fluorescein copolymer and methylrhodamine copolymer; and Senter et al., Photochem. Photobiol. 42:231 237, 1985; nitrobenzyloxy carbonyl chloride cross linking reagents that produce photocleavable linkages), relevant portions incorporated herein by reference. Such linkers are particularly useful in treating dermatological or ophthalmic conditions. In addition, other tissues, such as blood vessels that can be exposed to light using fiber-optics during angioplasty in the prevention or treatment of restenosis may benefit from the use of photocleavable linkers. After administration of the conjugate, the body part is exposed to light, resulting in release of the targeted moiety from the conjugate. Heat sensitive linkers would also have similar applicability.

In one embodiment, the linker has a structure

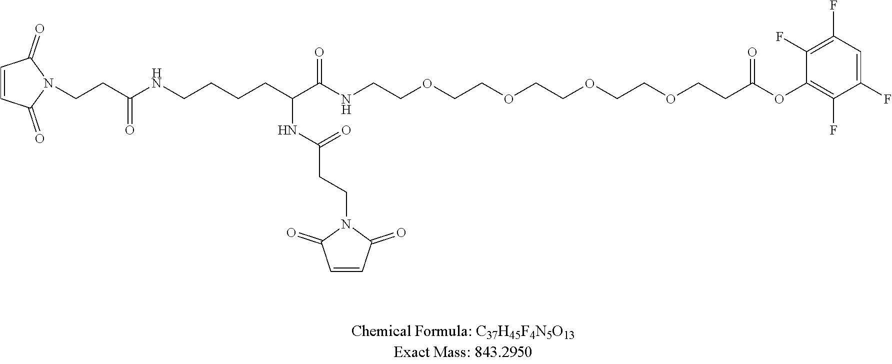 [SMILES: O=C(CCN1C(=O)C=CC1=O)NCCCCC(NC(=O)CCN1C(=O)C=CC1=O)C(=O)NCCOCCOCCOCCOCCC(=O)Oc1c(F)c(F)cc(F)c1F]

### Polynucleotides and Methods of Making

Also provided herein is any polynucleotide (e.g., DNA or RNA) encoding an anti-TfR protein-drug conjugate, e.g., anti-TfR scFv-drug conjugate or anti-TfR Fab-drug conjugate (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263) or polypeptide portion(s) thereof, optionally, which is operably linked to a promoter or other expression control sequence. Polypeptides encoded by such polynucleotides are also provided herein.

Nucleotide sequences of HCVRs and LCVRs of anti-hTfR protein-drug conjugates set forth herein are summarized below in Table 1-5. Polynucleotides encoding an anti-hTfR protein-drug conjugates, or polypeptide portion(s) thereof, that include one or more of the HCVRs and/or LCVRs set forth in Table 1-5 are also provided herein.

TABLE 1-5
Nucleotide Sequences encoding Domains in Antibodies,
Antigen-binding Fragments (e.g., Fabs or scFv Molecules)
in Described Protein-drug Conjugates.

| Anti-hTfR Molecule | HCVR | LCVR |
|---|---|---|
| 31874B | 1 | 6 |
| 31863B | 11 | 16 |
| 69348 | 21 | 26 |
| 69340 | 31 | 36 |
| 69331 | 41 | 46 |
| 69332 | 51 | 56 |
| 69326 | 61 | 66 |
| 69329 | 71 | 76 |
| 69323 | 81 | 86 |
| 69305 | 91 | 96 |
| 69307 | 101 | 106 |
| 12795B | 111 | 116 |
| 12798B | 121 | 126 |
| 12799B | 131 | 136 |
| 12801B | 141 | 146 |
| 12802B | 151 | 156 |
| 12808B | 161 | 166 |
| 12812B | 171 | 176 |
| 12816B | 181 | 186 |
| 12833B | 191 | 196 |
| 12834B | 201 | 206 |
| 12835B | 211 | 216 |
| 12847B | 221 | 226 |
| 12848B | 231 | 236 |
| 12843B | 241 | 246 |
| 12844B | 251 | 256 |
| 12845B | 261 | 266 |
| 12839B | 271 | 276 |
| 12841B | 281 | 286 |
| 12850B | 291 | 296 |
| 69261 | 301 | 306 |
| 69263 | 311 | 316 |

In some embodiments, the nucleic acid molecule comprises a polynucleotide sequence selected from any of the LCVR nucleic acid sequences listed in Table 1-2, or a substantially similar sequence thereof having at least 90%, at least 95%, at least 98% or at least 99% sequence identity thereto.

For example, provided herein is a polynucleotide encoding an anti-hTfR protein-drug conjugate, or polypeptide portion(s) thereof, that includes:
a polynucleotide encoding a HCVR that comprises the nucleotide sequence set forth in SEQ ID NO: 1, and a LCVR that comprises the nucleotide sequence set forth in SEQ ID NO: 6;
a polynucleotide encoding a HCVR that comprises the nucleotide sequence set forth in SEQ ID NO: 11, and a LCVR that comprises the nucleotide sequence set forth in SEQ ID NO: 16;
a polynucleotide encoding a HCVR that comprises the nucleotide sequence set forth in SEQ ID NO: 21, and a LCVR that comprises the nucleotide sequence set forth in SEQ ID NO: 26;
a polynucleotide encoding a HCVR that comprises the nucleotide sequence set forth in SEQ ID NO: 31, and a LCVR that comprises the nucleotide sequence set forth in SEQ ID NO: 36;
a polynucleotide encoding a HCVR that comprises the nucleotide sequence set forth in SEQ ID NO: 41, and a LCVR that comprises the nucleotide sequence set forth in SEQ ID NO: 46;
a polynucleotide encoding a HCVR that comprises the nucleotide sequence set forth in SEQ ID NO: 51, and a LCVR that comprises the nucleotide sequence set forth in SEQ ID NO: 56;
a polynucleotide encoding a HCVR that comprises the nucleotide sequence set forth in SEQ ID NO: 61, and a LCVR that comprises the nucleotide sequence set forth in SEQ ID NO: 66;
a polynucleotide encoding a HCVR that comprises the nucleotide sequence set forth in SEQ ID NO: 71, and a LCVR that comprises the nucleotide sequence set forth in SEQ ID NO: 76;
a polynucleotide encoding a HCVR that comprises the nucleotide sequence set forth in SEQ ID NO: 81, and a LCVR that comprises the nucleotide sequence set forth in SEQ ID NO: 86;
a polynucleotide encoding a HCVR that comprises the nucleotide sequence set forth in SEQ ID NO: 91, and a LCVR that comprises the nucleotide sequence set forth in SEQ ID NO: 96;
a polynucleotide encoding a HCVR that comprises the nucleotide sequence set forth in SEQ ID NO: 101, and a LCVR that comprises the nucleotide sequence set forth in SEQ ID NO: 106;
a polynucleotide encoding a HCVR that comprises the nucleotide sequence set forth in SEQ ID NO: 111, and a LCVR that comprises the nucleotide sequence set forth in SEQ ID NO: 116;
a polynucleotide encoding a HCVR that comprises the nucleotide sequence set forth in SEQ ID NO: 121, and a LCVR that comprises the nucleotide sequence set forth in SEQ ID NO: 126;
a polynucleotide encoding a HCVR that comprises the nucleotide sequence set forth in SEQ ID NO: 131, and a LCVR that comprises the nucleotide sequence set forth in SEQ ID NO: 136;
a polynucleotide encoding a HCVR that comprises the nucleotide sequence set forth in SEQ ID NO: 141, and a LCVR that comprises the nucleotide sequence set forth in SEQ ID NO: 146;
a polynucleotide encoding a HCVR that comprises the nucleotide sequence set forth in SEQ ID NO: 151, and a LCVR that comprises the nucleotide sequence set forth in SEQ ID NO: 156;
a polynucleotide encoding a HCVR that comprises the nucleotide sequence set forth in SEQ ID NO: 161, and a LCVR that comprises the nucleotide sequence set forth in SEQ ID NO: 166;
a polynucleotide encoding a HCVR that comprises the nucleotide sequence set forth in SEQ ID NO: 171, and a LCVR that comprises the nucleotide sequence set forth in SEQ ID NO: 176;
a polynucleotide encoding a HCVR that comprises the nucleotide sequence set forth in SEQ ID NO: 181, and a LCVR that comprises the nucleotide sequence set forth in SEQ ID NO: 186;
a polynucleotide encoding a HCVR that comprises the nucleotide sequence set forth in SEQ ID NO: 191, and a LCVR that comprises the nucleotide sequence set forth in SEQ ID NO: 196;
a polynucleotide encoding a HCVR that comprises the nucleotide sequence set forth in SEQ ID NO: 201, and a LCVR that comprises the nucleotide sequence set forth in SEQ ID NO: 206;
a polynucleotide encoding a HCVR that comprises the nucleotide sequence set forth in SEQ ID NO: 211, and a LCVR that comprises the nucleotide sequence set forth in SEQ ID NO: 216;
a polynucleotide encoding a HCVR that comprises the nucleotide sequence set forth in SEQ ID NO: 221, and a LCVR that comprises the nucleotide sequence set forth in SEQ ID NO: 226;
a polynucleotide encoding a HCVR that comprises the nucleotide sequence set forth in SEQ ID NO: 231, and a LCVR that comprises the nucleotide sequence set forth in SEQ ID NO: 236;
a polynucleotide encoding a HCVR that comprises the nucleotide sequence set forth in SEQ ID NO: 241, and a LCVR that comprises the nucleotide sequence set forth in SEQ ID NO: 246;
a polynucleotide encoding a HCVR that comprises the nucleotide sequence set forth in SEQ ID NO: 251, and a LCVR that comprises the nucleotide sequence set forth in SEQ ID NO: 256;
a polynucleotide encoding a HCVR that comprises the nucleotide sequence set forth in SEQ ID NO: 261, and a LCVR that comprises the nucleotide sequence set forth in SEQ ID NO: 266;
a polynucleotide encoding a HCVR that comprises the nucleotide sequence set forth in SEQ ID NO: 271, and a LCVR that comprises the nucleotide sequence set forth in SEQ ID NO: 276;
a polynucleotide encoding a HCVR that comprises the nucleotide sequence set forth in SEQ ID NO: 281, and a LCVR that comprises the nucleotide sequence set forth in SEQ ID NO: 286;
a polynucleotide encoding a HCVR that comprises the nucleotide sequence set forth in SEQ ID NO: 291, and a LCVR that comprises the nucleotide sequence set forth in SEQ ID NO: 296;
a polynucleotide encoding a HCVR that comprises the nucleotide sequence set forth in SEQ ID NO: 301, and a LCVR that comprises the nucleotide sequence set forth in SEQ ID NO: 306; or
a polynucleotide encoding a HCVR that comprises the nucleotide sequence set forth in SEQ ID NO: 311, and a LCVR that comprises the nucleotide sequence set forth in SEQ ID NO: 316;
wherein the HCVR and LCVR are in either order.

In some embodiments the nucleic acid molecule comprises a polynucleotide sequence selected from any of the HCDR1 nucleic acid sequences listed in Table 1-2, or a substantially similar sequence thereof having at least 90%, at least 95%, at least 98% or at least 99% sequence identity thereto.

In some embodiments the nucleic acid molecule comprises a polynucleotide sequence selected from any of the HCDR2 nucleic acid sequences listed in Table 1-2, or a substantially similar sequence thereof having at least 90%, at least 95%, at least 98% or at least 99% sequence identity thereto.

In some embodiments the nucleic acid molecule comprises a polynucleotide sequence selected from any of the HCDR3 nucleic acid sequences listed in Table 1-2, or a substantially similar sequence thereof having at least 90%, at least 95%, at least 98% or at least 99% sequence identity thereto.

In some embodiments the nucleic acid molecule comprises a polynucleotide sequence selected from any of the LCDR1 nucleic acid sequences listed in Table 1-2, or a substantially similar sequence thereof having at least 90%, at least 95%, at least 98% or at least 99% sequence identity thereto.

In some embodiments the nucleic acid molecule comprises a polynucleotide sequence selected from any of the LCDR2 nucleic acid sequences listed in Table 1-2, or a substantially similar sequence thereof having at least 90%, at least 95%, at least 98% or at least 99% sequence identity thereto.

In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide described herein. Promoters which may be used to control gene expression include, but are not limited to, cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the beta-lactamase promoter (VIIIa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Ga/4 promoter, the ADC (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

Polynucleotides described herein may include polynucleotides encoding polypeptide chains which are variants of those whose nucleotide sequence is specifically set forth herein. A “variant” of a polynucleotide refers to a polynucleotide comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence that is set forth herein (see e.g., the nucleotide sequences of Table 1-5); when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1, −2; gap costs: linear). In an embodiment, a variant of a nucleotide sequence specifically set forth herein comprises one or more (e.g., 1, 2, 3, 4, 5, 6, 7, 8, 9, 10, 11 or 12) point mutations, insertions (e.g., in frame insertions) or deletions (e.g., in frame deletions) of one or more nucleotides. Such mutations may, in an embodiment, be missense or nonsense mutations.

Eukaryotic and prokaryotic host cells, including mammalian cells, may be used as hosts for expression of polypeptide portion(s) (e.g., antigen-binding protein) of an anti-TfR protein-drug conjugate. Such host cells are well known in the art and many are available from the American Type Culture Collection (ATCC). These host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Further provided herein is an isolated host cell (e.g., a CHO cell or any type of host cell set forth above) comprising an anti-TfR protein-drug conjugate such as 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263.

Also provided herein is a cell which is expressing TfR or an antigenic fragment or fusion thereof which is bound by an antigen-binding protein described herein (e.g., an antibody or antigen-binding fragment thereof), for example, wherein the cell is in the body of a subject or is in vitro. In addition, the present disclosure also provides a complex comprising a TfR-binding protein, e.g., antibody or antigen-binding fragment thereof, as discussed herein complexed with TfR polypeptide or an antigenic fragment thereof or fusion thereof and/or with a secondary antibody or antigen-binding fragment thereof (e.g., detectably labeled secondary antibody) that binds specifically to the anti-TfR antibody or fragment. In an embodiment, the complex is in vitro (e.g., is immobilized to a solid substrate) or is in the body of a subject.

In an embodiment, a myc tag has the amino acid sequence EQKLISEEDLGG (SEQ ID NO: 500), a His₆ or hexahis or hexahistidine tag has the amino acid sequence HHHHHH (SEQ ID NO: 501), an mmh tag has the amino acid sequence EQKLISEEDLGGEQKLISEEDLHHHHHH (SEQ ID NO: 461) and a mouse Fc tag has the amino acid sequence EPRGPTIKPCPPCKCPAPNLLGGPSVFIFPPKIKDVLMISLSPIVTCVVVDVSEDDPDVQI SWFVNNVEVHTAQTQTHREDYNSTLRVVSALPIQHQDWMSGKEFKCKVNNKDLPAPI ERTISKPKGSVRAPQVYVLPPPEEEMTKKQVTLTCMVTDFMPEDIYVEWTNNGKTELN YKNTEPVLDSDGSYFMYSKLRVEKKNWVERNSYSCSVVHEGLHNHHTTKSFSRTPGK (SEQ ID NO: 502).

In addition, provided herein is a complex comprising an anti-TfR protein-drug conjugate, as discussed herein complexed with a transferrin receptor polypeptide or an antigenic fragment thereof or fusion thereof and/or with a secondary antibody or antigen-binding fragment thereof (e.g., detectably labeled secondary antibody) that binds specifically to the anti-TfR protein-drug conjugate. In an embodiment, the complex is in vitro (e.g., is immobilized to a solid substrate) or is in the body of a subject.

Anti-TfR antigen-binding proteins in the protein-drug conjugates disclosed herein may be produced recombinantly in an E. coli/T7 expression system. In this embodiment, polynucleotides encoding the anti-TfR antigen-binding proteins described herein (e.g., 31874B; 31863B; 69348; 69340; 69331; 69332; 69326; 69329; 69323; 69305; 69307; 12795B; 12798B; 12799B; 128011B; 12802B; 12808B; 12812B; 12816B; 12833B; 12834B; 12835B; 12847B; 12848B; 12843B; 12844B; 12845B; 12839B; 128411B; 12850B; 69261; or 69263) may be inserted into a pET-based plasmid and expressed in the E. coli/T7 system. For example, methods for expressing anti-TfR antigen-binding proteins in a host cell (e.g., bacterial host cell such as E. coli such as BL21 or BL21 DE3) may comprise expressing T7 RNA polymerase in the cell which also includes a polynucleotide encoding the anti-TfR antigen-binding protein that is operably linked to a T7 promoter. For example, in an embodiment, a bacterial host cell, such as an E. coli, includes a polynucleotide encoding the T7 RNA polymerase gene operably linked to a lac promoter and expression of the polymerase and the chain is induced by incubation of the host cell with IPTG (isopropyl-beta-D-thiogalactopyranoside). See U.S. Pat. Nos. 4,952,496 and 5,693,489 or Studier & Moffatt, Use of bacteriophage T7 RNA polymerase to direct selective high-level expression of cloned genes, J. Mol. Biol. 1986 May 5; 189(1): 113-30.

There are several methods by which to produce recombinant antibodies which are known in the art. One example of a method for recombinant production of antibodies is disclosed in U.S. Pat. No. 4,816,567.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, polynucleotides may be introduced into mammalian cells by viral vectors. Methods of transforming cells are well known in the art. See, for example, U.S. Pat. Nos. 4,399,216; 4,912,040; 4,740,461 and 4,959,455. Thus, recombinant methods for making an anti-TfR antigen-binding proteins described herein may comprise (i) introducing, into a host cell, one or more polynucleotides encoding the anti-TfR antigen-binding proteins, for example, wherein the polynucleotide is in a vector; and/or integrates into the host cell chromosome and/or is operably linked to a promoter; (ii) culturing the host cell (e.g., CHO or Pichia or Pichia pastoris) under conditions favorable to expression of the polynucleotide and, (iii) optionally, isolating the anti-TfR antigen-binding proteins from the host cell and/or medium in which the host cell is grown. When making an antigen-binding protein (e.g., antibody or antigen-binding fragment) comprising more than one immunoglobulin chain, e.g., an antibody that comprises two heavy immunoglobulin chains and two light immunoglobulin chains, co-expression of the chains in a single host cell leads to association of the chains, e.g., in the cell or on the cell surface or outside the cell if such chains are secreted, so as to form the antigen-binding protein (e.g., antibody or antigen-binding fragment). The methods described herein include those wherein only a heavy immunoglobulin chain or only a light immunoglobulin chain or both (e.g., any of those discussed herein including mature fragments and/or variable domains thereof) are expressed in a cell. Such single chains are useful, for example, as intermediates in the expression of an antibody or antigen-binding fragment that includes such a chain. For example, also included herein are anti-TfR antigen-binding proteins which are the product of the production methods set forth herein, and, optionally, the purification methods set forth herein.

In an embodiment, a method for making an anti-TfR antigen-binding proteins, includes a method of purifying the anti-TfR antigen-binding proteins, e.g., by column chromatography, precipitation and/or filtration. As discussed, the product of such a method also is also provided herein.

### Preparation of Human Antibodies

The anti-TfR antibodies and antigen-binding fragments described herein can be fully human antibodies and fragments. Methods for generating monoclonal antibodies, including fully human monoclonal antibodies are known in the art. Any such known methods can be used in the methods described herein to make human antibodies that specifically bind to TfR.

Using VELOCIMMUNE™ technology, for example, or any other similar known method for generating fully human monoclonal antibodies, high affinity chimeric antibodies to TfR are initially isolated having a human variable region and a mouse constant region. As in the experimental section below, the antibodies are characterized and selected for desirable characteristics, including affinity, ligand blocking activity, selectivity, epitope, etc. If necessary, mouse constant regions are replaced with a desired human constant region, for example wild-type or modified IgG1 or IgG4, to generate a fully human anti-TfR antibody. While the constant region selected may vary according to specific use, high affinity antigen-binding and target specificity characteristics reside in the variable region. In certain instances, fully human anti-TfR antibodies are isolated directly from antigen-positive B cells. See, for example, U.S. Pat. No. 6,596,541, Regeneron Pharmaceuticals, VELOCIMMUNE®.

### Treatment and Administration

Provided herein are anti-TfR protein-drug conjugates (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or69263) which can be used, for example, for delivering a molecular cargo to the body of a subject, e.g., for treating or preventing a disease or disorder in the body (e.g., the brain or muscle) of the subject.

In one aspect, provided herein is a pharmaceutical composition comprising protein-drug conjugate together with a pharmaceutically acceptable carrier and/or excipient. The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The pharmaceutical compositions described herein may be in any suitable form (depending upon the desired method of administering to a patient). Suitable compositions and methods of administration are known to those skilled in the art, for example see, Johnson et al., Blood. 2009; 114(3):535-46.

The pharmaceutical compositions may comprise the protein-drug conjugates described herein either in the free form or in the form of a pharmaceutically acceptable salt. The term “pharmaceutically acceptable salt” as used herein refers to a derivative of the disclosed protein-drug conjugates wherein the protein-drug conjugates is modified by making acid or base salts of the agent. For example, acid salts are prepared from the free base (typically wherein the neutral form of the drug has a neutral —NH₂ group) involving reaction with a suitable acid. Suitable acids for preparing acid salts include both organic acids, e.g., acetic acid, benzoic acid, citric acid, propionic acid, glycolic acid, trifluoroacetic acid, pyruvic acid, oxalic acid, malic acid, malonic acid, maleic acid, succinic acid, fumaric acid, tartaric acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid phosphoric acid and the like. Conversely, preparation of basic salts of acid moieties which may be present on a protein-drug conjugates are prepared using a pharmaceutically acceptable base such as sodium hydroxide, potassium hydroxide, ammonium hydroxide, calcium hydroxide, trimethylamine or the like.

In some embodiments, the protein-drug conjugates described herein may be present in a solution at a concentration of about 1 μg/mL to 50 mg/mL, for example, about 0.1 mg/mL to 10 mg/mL, about 0.2 mg/mL to 5 mg/mL, about 0.5 mg/mL to 8 mg/mL, about 0.8 mg/mL to 12 mg/mL, about 1 mg/mL to 15 mg/mL, about 2 mg/mL to 20 mg/mL, or about 5 mg/mL to 25 mg/mL, or about 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 1.25 mg/mL, 1.5 mg/mL, 1.75 mg/mL, 2 mg/mL, 2.25 mg/mL, 2. 5 mg/mL, 2.75 mg/mL, 3 mg/mL, 3.25 mg/mL, 3. 5 mg/mL, 3.75 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL or 20 mg/mL.

The pharmaceutical composition may be adapted for administration by any appropriate route such as, e.g., parenteral (including subcutaneous, intramuscular, or intravenous), intrathecal, intracerebroventricular, intraparenchymal injections into the central nervous system, enteral (including oral or rectal), inhalation, or intranasal routes.

Such compositions may be prepared by any method known in the art of pharmacy, for example, by mixing the active ingredient with the carrier(s) or excipient(s) under sterile conditions.

In addition, disclosed herein are pharmaceutical dosage forms comprising the protein-drug conjugates described herein.

Pharmaceutical compositions based on the protein-drug conjugates disclosed herein can be formulated in any conventional manner using one or more physiologically acceptable carriers and/or excipients. The protein-drug conjugates may be formulated for administration by, for example, injection, inhalation, or insulation (either through the mouth or the nose) or by oral, buccal, parenteral or rectal administration, or by administration directly to an organ or tissue.

The pharmaceutical compositions can be formulated for a variety of modes of administration, including systemic, topical, or localized administration. Techniques and formulations can be found in, for example, Remington's Pharmaceutical Sciences, Meade Publishing Co., Easton, Pa. For systemic administration, injection is preferred, including intramuscular, intravenous, intraperitoneal, and subcutaneous. For the purposes of injection, the pharmaceutical compositions can be formulated in liquid solutions, preferably in physiologically compatible buffers, such as Hank's solution or Ringer's solution. In addition, the pharmaceutical compositions may be formulated in solid form and redissolved or suspended immediately prior to use. Lyophilized forms of the pharmaceutical composition are also suitable.

In some embodiments, the pharmaceutical compositions described herein may be lyophilized. As a non-limiting example, the obtained lyophilizate can be reconstituted into a hydrous composition by adding a hydrous solvent. In some embodiments, the hydrous composition may be able to be directly administered parenterally (e.g., intravenously) to a patient. Therefore, a further embodiment is a hydrous pharmaceutical composition, obtainable via reconstitution of the lyophilizate with a hydrous solvent.

In some embodiments, the pharmaceutical composition disclosed herein may comprise a lyophilized formulation. As a non-limiting example, the lyophilization formulation may comprise protein-drug conjugates described herein, mannitol, and/or TWEEN 80®. As another non-limiting example, the lyophilization formulation may comprise the protein-drug conjugates disclosed herein, mannitol and poloxamer 188. In some embodiments, the pharmaceutical composition may comprise a lyophilization formulation comprising a reconstituted-liquid composition.

In some embodiments, pharmaceutical compositions described herein may provide a formulation with an enhanced solubility and/or moistening of the lyophilizate over previously known compositions. As a non-limiting example, enhanced solubility and/or moistening of the lyophilizate may be achieved using an appropriate composition of excipients. In this way, pharmaceutical compositions described herein comprising protein-drug conjugates described herein may be developed to show a desired shelf stability at (e.g., at −20° C., +5° C., or +25° C.) and can be easily resolubilized such that the lyophilizate can be completely dissolved through the use of a buffer or other excipients from seconds up to two or more minutes, with or without the use of an of ultrasonic homogenizer. Furthermore, the composition can be easily provided to a patient in need of treatment via any appropriate delivery route disclosed herein, e.g., parenteral (including subcutaneous, intramuscular, or intravenous), enteral (including oral or rectal), inhalation, or intranasal routes. As a non-limiting example, the pH-value of the resulting solution may be between pH 2.7 and pH 9.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulfate). The tablets can also be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. ationd oil, oily esters, ethyl alcohol or fractionated vegetable oils); and preservatives (e.g. methyl or propyl-p-hydroxybenzoates or sorbic acid). The preparations can also contain buffer salts, flavoring, coloring and sweetening agents as appropriate.

The pharmaceutical compositions can be formulated for parenteral administration by injection, e.g. by bolus injection or continuous infusion. Formulations for injection can be presented in a unit dosage form, e.g. in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

Additionally, the pharmaceutical compositions can also be formulated as a depot preparation. These long-acting formulations can be administered by implantation (e.g. subcutaneously or intramuscularly) or by intramuscular injection. Thus, for example, the compounds may be formulated with suitable polymeric or hydrophobic materials (e.g. as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology can include microspheres having a precapillary size, which can be injected via a coronary catheter into any selected part of an organ without causing inflammation or ischemia. The administered therapeutic is men slowly released from the microspheres and absorbed by the surrounding cells present in the selected tissue.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts, and fusidic acid derivatives. In addition, detergents may be used to facilitate permeation. Transmucosal administration can occur using nasal sprays or suppositories. For topical administration, the vector particles described herein can be formulated into ointments, salves, gels, or creams as generally known in the art. A wash solution can also be used locally to treat an injury or inflammation in order to accelerate healing.

Pharmaceutical forms suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g. refrigeration and freezing) and must be preserved against the contaminating action of microorganisms, such as bacteria and fungi.

A protein-drug conjugate described herein can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutically acceptable carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents known in the art. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating the active compounds or constructs in the required amount in the appropriate solvent with various of the other ingredients enumerated above, as required, followed by filtered sterilization.

Upon formulation, solutions can be administered in a manner compatible with the dosage formulation and in such amount as is therapeutically effective. The formulations are easily administered in a variety of dosage forms, such as the type of injectable solutions described above, but slow-release capsules or microparticles and microspheres and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intratumorally, intramuscular, subcutaneous and intraperitoneal administration. In this context, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion.

The person responsible for administration will, in any event, determine the appropriate dose for the individual subject. For example, a subject may be administered the protein-drug conjugates described herein on a daily or weekly basis for a time period or on a monthly, bi-yearly or yearly basis.

In addition to the compounds formulated for parenteral administration, such as intravenous, intratumorally, intradermal or intramuscular injection, other pharmaceutically acceptable forms include, e.g., tablets or other solids for oral administration; liposomal formulations; time release capsules; biodegradable and any other form currently used.

One may also use intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 7.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines and are used for asthma prophylaxis.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will include an inert diluent or assimilable edible carrier, or they may be enclosed in hard or soft-shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup of elixir may contain the active compounds sucrose as a sweetening agent methyl and propylparabens as preservatives, a dye and flavoring, such as cherry or orange flavor.

Compositions may include administration to a subject intravenously, intratumorally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a cream, or in a lipid composition.

Compositions as disclosed herein can also include adjuvants such as aluminum salts and other mineral adjuvants, tensoactive agents, bacterial derivatives, vehicles and cytokines. Adjuvants can also have antagonizing immunomodulating properties. Compositions and methods as disclosed herein can also include adjuvant therapy.

The pharmaceutical compositions described herein may be administered directly into the patient, into the affected organ or systemically i.d., i.m., s.c., i.p. and i.v., or applied ex vivo to cells derived from the patient or a human cell line which are subsequently administered to the patient, or used in vitro to select a subpopulation of cells derived from the patient, which are then re-administered to the patient.

In some embodiments, anti-TfR protein-drug conjugates described herein are used for treating or preventing a disease or disorder, such as but not limited to, a lysosomal storage disease and disorder, a heart disease or disorder, a central nervous system (CNS) disease or disorder, an eye disease or disorder, a brain disease or disorder, a spinal cord disease or disorder, a peripheral nervous system (PNS) disease or disorder, a muscular disease or disorder (e.g., skeletal muscle disease or disorder), a cartilage disease or disorder, a bone growth plate disease or disorder, a kidney disease or disorder, and a blood disease or disorder.

In some embodiments, anti-TfR protein-drug conjugates described herein are used for treating or preventing a neurological disease or disorder. Examples of neurological diseases or disorders include, but are not limited to, lysosomal storage diseases, amyloidosis, neuropathy, neurodegenerative diseases, leukodystrophy, neuropsychiatric diseases, traumatic brain injury, neurodevelopmental diseases, and neuromuscular diseases, seizure, behavioral disorders, ocular diseases or disorders, viral or microbial infections, inflammation, ischemia, and cancer. Specific examples of neurological disorders include, but are not limited to, neurodegenerative diseases (e.g., Lewy body disease, olivopontocerebellar atrophy, multiple system atrophy, postpoliomyelitis syndrome, Parkinson's disease, striatonigral degeneration, Shy-Draeger syndrome, tauopathies (e.g., Alzheimer disease and supranuclear palsy)), prion diseases (e.g., bovine spongiform encephalopathy, kuru, Creutzfeldt-Jakob syndrome, Gerstmann-Straussler-Scheinker disease, chronic wasting disease, fatal familial insomnia, and scrapie), bulbar palsy, motor neuron disease, and nervous system heterodegenerative disorders (e.g., Alexander's disease, Cockayne syndrome, Canavan disease, hepatolenticular degeneration, Huntington's disease, Halervorden-Spatz syndrome, neuronal ceroid-lipofuscinosis, Turette's syndrome, Menkes kinky hair syndrome, Iafora disease, Rett syndrome, Lesch-Nyhan syndrome, and Unverricht-Lundborg syndrome), dementia (e.g., Pick's disease and spinocerebellar ataxia), cancer (e.g., central nervous system (CNS) cancers, including brain metastases resulting from cancer elsewhere in the body).

In some embodiments, anti-TfR protein-drug conjugates described herein are used for treating or preventing a muscular disease or disorder. Examples of neurological diseases or disorders include, but are not limited to, centronuclear myopathy (CNM), Duchenne muscular dystrophy (DMD), facioscapulohumeral muscular dystrophy (FSHD), familial hypertrophic cardiomyopathy, Fibrodysplasia Ossificans Progressiva (FOP), Friedreich's ataxia (FRDA), inclusion body myopathy 2, Iaing distal myopathy, myofibrillar myopathy, myotonia congenita (e.g., Thomsen Disease), myotonic dystrophy type I, myotonic dystrophy type II, myotubular myopathy, oculopharyngeal muscular dystrophy, Pompe disease, glycogen storage disease, paramyotonia congenita, and muscle atrophy. In some embodiments, the muscular disease or disorder is muscle atrophy. Muscle atrophy may be due to a chronic illness, including acquired immunodeficiency syndrome (AIDS), congestive heart failure, cancer, chronic obstructive pulmonary disease, and renal failure, or muscle disuse.

In some embodiments, anti-TfR protein-drug conjugates described herein are used for treating or preventing a muscular disease or disorder which is Myotonic dystrophy, Duchenne muscular dystrophy (DMD), Fascioscapulohumeral muscular dystrophy (e.g., Type 1), or muscle atrophy (cachexia, sarcopenia).

Myotonic muscular dystrophy is a multi-system disorder that affects the skeletal muscles (the muscles that move the limbs and trunk) as well as smooth muscles (the muscles that control the digestive system) and cardiac muscles of the heart. Symptoms of myotonic dystrophy might include difficulty releasing one's grip (myotonia), weakness of muscles in the hands and feet, difficulty swallowing and abnormal heart rhythms. Non-muscle symptoms may also include learning difficulties, daytime sleepiness, infertility and early cataracts. There are two known forms of this disease (Myotonic Dystrophy Type 1 (DM1) and Myotonic Dystrophy Type 2 (DM2)). Both are caused by abnormal expansions of repeated areas of genes. In Myotonic Dystrophy Type 1, the repeat expansion enlarges with each generation, frequently leading to earlier onset and increased severity of symptoms with each affected generation. Myotonic Dystrophy Type 1 therefore frequently affects children in families with this disorder. In DM1, the abnormal DNA expansion is in the DMPK (dystrophia myotonica protein kinase) gene. An underlying cause of DM2 is an expanded DNA section in the ZNF9 (zinc finger 9) gene, also known as CNBP gene. Thus, the methods provided herein include methods for treating or preventing myotonic muscular dystrophy (DM), for example, DM1 or DM2, by administering an antigen-binding protein conjugated to a molecular cargo which is effective at treating DM, e.g., an interfering RNA (e.g., siRNA or antisense oligonucleotide) that targets the mutant DMPK or CNBP gene.

Duchenne muscular dystrophy (DMD) is a genetic disorder characterized by progressive muscle degeneration and weakness due to the alterations of a protein called dystrophin that helps keep muscle cells intact. Muscle weakness is the principal symptom of DMD. DMD symptom onset is typically in early childhood, usually between ages 2 and 3. The disease primarily affects boys, but in rare cases it can affect girls. Thus, the methods provided herein include methods for treating or preventing DMD, by administering an antigen-binding protein conjugated to a molecular cargo which is effective at treating DMD, e.g., an interfering RNA (e.g., siRNA or antisense oligonucleotide) that targets the mutant dystrophin gene.

Facioscapulohumeral muscular dystrophy (FSHD) typically presents with weakness of the facial muscles, the stabilizers of the scapula, or the dorsiflexors of the foot. Severity is highly variable. Weakness is slowly progressive and approximately 20% of affected individuals eventually require a wheelchair. Chromosome 4 contains D4Z4 repeat units. In someone with FSHD, the number of D4Z4 repeat units is reduced, which causes DUX4 to be unnecessarily activated and produce DUX4 protein. DUX4 protein is thought to contribute to muscle wasting, inflammation and damage inside the muscle cells of someone with FSHD. The methods provided herein include methods for treating or preventing FSHD, by administering an antigen-binding protein conjugated to a molecular cargo which is effective at treating FSHD, e.g., an interfering RNA (e.g., siRNA or antisense oligonucleotide) that targets the mutant DUX4 gene.

Moreover, the methods provided herein include methods for treating or preventing muscle atrophy conditions or metabolic diseases (e.g., cachexia or sarcopenia), by administering an antigen-binding protein conjugated to a molecular cargo which is effective at treating such a condition or disease, e.g., a growth factor, neurotrophic factor, disease modifying muscle protein, and/or metabolic protein.

In some embodiments, the methods provided herein include methods for treating or preventing a neurodegenerative disease such as Alzheimer's disease, Huntington's disease, amyotrophic lateral sclerosis, Parkinson's disease, or prion disease.

In one embodiment, provided herein is a method for treating or preventing Alzheimer's disease by administering an antigen-binding protein conjugated to a molecular cargo which is effective at treating Alzheimer's disease, e.g., an interfering RNA (e.g., siRNA or antisense oligonucleotide) that targets the ApoE gene (encoding apolipoprotein E), MAPT gene (encoding microtubule-associated protein tau), or APP gene (encoding amyloid precursor protein), or a mutant thereof.

In one embodiment, provided herein is a method for treating or preventing Huntington's disease by administering an antigen-binding protein conjugated to a molecular cargo which is effective at treating Huntington's disease, e.g., an interfering RNA (e.g., siRNA or antisense oligonucleotide) that targets the HTT gene (encoding Huntingtin), or a mutant thereof.

In one embodiment, provided herein is a method for treating or preventing amyotrophic lateral sclerosis by administering an antigen-binding protein conjugated to a molecular cargo which is effective at treating amyotrophic lateral sclerosis, e.g., an interfering RNA (e.g., siRNA or antisense oligonucleotide) that targets the SOD1 gene (encoding superoxide dismutase type 1), or C9orf72 gene (chromosome 9 open reading frame 72), or a mutant thereof.

In one embodiment, provided herein is a method for treating or preventing Parkinson's disease by administering an antigen-binding protein conjugated to a molecular cargo which is effective at treating Parkinson's disease, e.g., an interfering RNA (e.g., siRNA or antisense oligonucleotide) that targets the SNCA gene (encoding alpha-synuclein), or LRRK2 gene (encoding Leucine-rich repeat kinase 2), or a mutant thereof.

In one embodiment, provided herein is a method for treating or preventing prion disease by administering an antigen-binding protein conjugated to a molecular cargo which is effective at treating prion disease, e.g., an interfering RNA (e.g., siRNA or antisense oligonucleotide) that targets the PRNP gene (encoding the prion protein), or a mutant thereof.

In some embodiments, the methods provided herein include methods for treating or preventing a heart disease or disorder, such as heart failure. In one embodiment, provided herein is a method for treating or preventing heart failure by administering an antigen-binding protein conjugated to a molecular cargo which is effective at treating prion disease, e.g., an interfering RNA (e.g., siRNA or antisense oligonucleotide) that targets the SLC5A1 gene (encoding solute carrier family 5 member 1), SLC16A3 gene (encoding solute carrier family 16 member 3), HDAC6 gene (encoding histone deacetylase), MMP27 gene (encoding matrix metallopeptidase 27), MFAP5 gene (encoding a 25-kD microfibril-associated glycoprotein), FAM64A gene (encoding family with sequence similarity 64 member A, also known as RCS1, PIMREG, or CATS), BAIAP3 gene (encoding BAI1 associated protein 3), MYH7 gene (encoding beta-myosin heavy chain), TPM1 gene (encoding tropomyosin 1), RBM20 gene (encoding RNA binding motif protein 20), KLHL24 gene (encoding kelch like family member 24), MYL2 gene (encoding myosin regulatory light chain 2), TNNT2 gene (encoding cardiac troponin T), or a mutant thereof. In some embodiments, the interfering RNA (e.g., siRNA or antisense oligonucleotide) mediates knockdown of the SLC5A1, SLC16A3, HDAC6, MMP27, MFAP5, FAM64A, or BAIAP3 gene. In some embodiments, the interfering RNA (e.g., siRNA or antisense oligonucleotide) mediates allele-specific mRNA silencing (targeting dominant-negative mutations) in the MYH7, TPM1, RBM20, KLHL24, MYL2, or TNNT2 gene.

In some embodiments, anti-TfR protein-drug conjugates described herein are used for treating or preventing a lysosomal storage disorder. “Lysosomal storage diseases” (LSDs) include any disorder resulting from a defect in lysosome function. The most well-known lysosomal disease includes Tay-Sachs, Gaucher, and Niemann-Pick disease. The pathogeneses of the diseases are ascribed to the buildup of incomplete degradation products in the lysosome, sometimes due to loss of protein function. Lysosomal storage diseases may be caused by loss-of-function or attenuating variants in the proteins whose normal function is to degrade or coordinate degradation of lysosomal contents. The proteins affiliated with lysosomal storage diseases include enzymes, receptors and other transmembrane proteins (e.g., NPC1), post-translational modifying proteins (e.g., sulfatase), membrane transport proteins, and non-enzymatic cofactors and other soluble proteins (e.g., GM2 ganglioside activator). Thus, lysosomal storage diseases encompass more than those disorders caused by defective enzymes per se, and include any disorder caused by any molecular defect.

LSDs include sphingolipidoses (heterogeneous group of inherited disorders of lipid metabolism affecting primarily the central nervous system), a mucopolysaccharidoses (a group of inherited lysosomal storage disorders), and glycogen storage diseases. In some embodiments, the LSD is any one or more of Fabry disease, Gaucher disease type I, Gaucher disease type II, Gaucher disease type III, Niemann-Pick disease type A, Niemann-Pick disease type BGM1-gangliosidosis, Sandhoff disease, Tay-Sachs disease, GM2-activator deficiency, GM3-gangliosidosis, metachromatic leukodystrophy, sphingolipid-activator deficiency, Schele disease, Hurler-Sceie disease, Hurler disease, Hunter disease, Sanfilippo A, Sanfilippo B, Sanfilippo C, Sanfilippo D, Morquio syndrome A, Morquio syndrome B, Maroteaux-Lamy disease, Sly disease, MPS IX, and Pompe disease. Thus, methods described herein also include method for treating or preventing any such LSD in a patient by administering an effective amount of anti-TfR protein-drug conjugate to the patient.

The nature of the molecular lesion in a lysosomal storage disease affects the severity of the disease in many cases, i.e., complete loss-of-function may be associated with pre-natal or neo-natal onset, and involves severe symptoms; partial loss-of-function may be associated with milder (relatively) and later-onset disease. Only a small percentage of activity may need to be restored to have to correct metabolic defects in deficient cells. Lysosomal storage diseases are generally described in Desnick & Schuchman, “Enzyme replacement therapy for lysosomal diseases: lessons from 20 years of experience and remaining challenges,” 13 Annu. Rev. Genomics Hum. Genet. 307-35, 2012).

Other non-limiting examples of lysosomal storage diseases include: sphingolipidoses; ceramidase deficiency, such as Farber disease, Krabbe disease (e.g., infantile onset or late onset); galactosialidosis; gangliosidoses, such as alpha-galactosidase (e.g., Fabry disease, or Schindler disease), beta-galactosidase/GM1 gangliosidosis (e.g., infantile, juvenile or adult/chronic), GM2 gangliosidosis (e.g., AB variant, activator deficiency, Sandhoff disease (e.g., infantile, juvenile or adult inset); Tay-Sachs disease (e.g., juvenile hexosaminidase A deficiency, chronic hexosaminidase A deficiency); glucocerebrosidase deficiency, such as Gaucher disease (e.g., Type I, Type II, Type III); sphingomyelinase deficiency, such as lysosomal acid lipase deficiency (e.g., early onset or late onset), and Niemann-Pick disease (e.g., Type A or Type B); sulfatidosis, such as metachromatic leukodystrophy (e.g., Saposin B deficiency), and multiple sulfatase deficiency; mucopolysaccharidoses, including Type I (e.g., MPS I Hurler syndrome, MPS I S Scheie syndrome, MPS I H—S Hurler-Scheie syndrome), Type II (Hunter syndrome), Type III (Sanfilippo syndrome) (e.g., MPS III A (Type A), MPS III B (Type B), MPS III C (Type C), MPS III D (Type D)), Type IV (Morquio) (e.g., MPS IVA (Type A), MPS IVB (Type B)), Type VI (Maroteaux-Lamy syndrome), Type VII (Sly syndrome), and Type IX (hyaluronidase deficiency); mucolipidosis, including Type I (sialidosis), Type II (I-cell disease), Type III (pseudo-Hurler polydystrophy /phosphotransferase deficiency), and Type IV (mucolipidin 1 deficiency); lipidoses, including Niemann-Pick disease (e.g., type C, type D), neuronal ceroid lipofuscinoses (e.g., Type 1 Santavuori-Haltia disease/infantile NCL (CLN1 PPT1), Type 2 Jansky-Bielschowsky disease/late infantile NCL (CLN2/LINCL TPP1), Type 3 Batten-Spielmeyer-Vogt disease/juvenile NCL (CLN3), Type 4 Kufs disease/adult NCL (CLN4), Type 5 Finnish Variant/late infantile (CLN5), Type 6 Late infantile variant (CLN6), Type 7 CLN7, Type 8 Northern epilepsy (CLN8), Type 8 Turkish late infantile (CLN8), Type 9 German/Serbian late infantile (unknown), Type 10 Congenital cathepsin D deficiency (CTSD)), and Wolman disease; glycoprotein disorders, such as alpha-mannosidosis, beta-mannosidosis, aspartylglucosaminuria, or fucosidosis; Lysosomal transport diseases, such as cystinosis, pycnodysostosis, Salla disease/sialic acid storage disease, and infantile free sialic acid storage disease; glycogen storage diseases, such as Type II Pompe disease, Type Ilb Danon disease; and other types such as cholesteryl ester storage disease.

The symptoms of lysosomal storage diseases can vary depending on the particular disorder and other variables such as the age of onset, and can be mild to severe. The symptoms can include developmental delay, seizures, dementia, movement disorders, deafness, and/or blindness. Some individuals with lysosomal storage diseases may have enlarged livers or spleens, pulmonary and cardiac problems, and bones that grow abnormally.

Patients with lysosomal storage diseases can be screened by a biochemical assay such as an assay that determines the level of enzyme(s) associated with a particular lysosomal storage disease. In some families where the disease-causing mutations are known, and in certain genetic isolates, mutation analysis may be performed. In addition, after a diagnosis is made by biochemical means, mutation analysis may be performed for certain disorders.

Accordingly, the methods provided herein include methods for treating or preventing a disease or disorder, such as a neurological or muscular disease or disorder described above, in a subject in need thereof, by administering a therapeutically effective amount of an anti-TfR protein-drug conjugate (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263) to the subject.

As used herein, the term “subject” refers to a mammal (e.g., rat, mouse, cat, dog, cow, sheep, horse, goat, rabbit), preferably a human, for example, in need of prevention and/or treatment of a disease or disorder, such as a neurological or muscular disease or disorder described herein. In an embodiment, a subject has been diagnosed as suffering from a disease or disorder, such as a neurological or muscular disease or disorder described herein.

Further disclosed herein are combinations including an anti-TfR protein-drug conjugate described herein (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263), in association with one or more further therapeutic agents. The anti-TfR protein-drug conjugate and the further therapeutic agent can be in a single composition or in separate compositions. For example, in an embodiment, the further therapeutic agent is alglucosidase alfa (e.g., Myozyme or Lumizyme), Rituximab, Methotrexate, Intravenous immunoglobulin (IVIG), avalglucosidase alfa-ngpt (e.g., Nexviazyme), a selective beta agonist (e.g., levalbuterol), an antibiotic, a steroid (e.g., cortisone or prednisone), a bisphosphonate, an infectious disease treatment (e.g., an antibiotic, a vaccine (e.g., Pneumococcal vaccine), palivizumab)

Methods for treating or preventing a disease or disorder in a subject in need of said treatment or prevention by administering an anti-TfR protein-drug conjugate, e.g., 12799B, 12839B, 12843B or 12847B, in association with a further therapeutic agent are also provided. Compositions comprising the anti-TfR protein-drug conjugate in association with one or more further therapeutic agents also form part described herein.

The term “in association with” indicates that components, an anti-TfR protein-drug conjugate described herein, along with another agent such as methotrexate, can be formulated into a single composition, e.g., for simultaneous delivery, or formulated separately into two or more compositions (e.g., a kit including each component). Each component can be administered to a subject at a different time than when the other component is administered; for example, each administration may be given non-simultaneously (e.g., separately or sequentially) at intervals over a given period of time. Moreover, the separate components may be administered to a subject by the same or by a different route.

An effective or therapeutically effective dose of anti-TfR protein-drug conjugate described herein for treating or preventing a disease or disorder, such as a neurological or muscular disease or disorder described herein, refers to the amount of anti-TfR protein-drug conjugate sufficient to alleviate one or more signs and/or symptoms of the disease or condition in the treated subject, whether by inducing the regression or elimination of such signs and/or symptoms or by inhibiting the progression of such signs and/or symptoms.

A symptom is a manifestation of disease apparent to the patient himself, while a sign is a manifestation of disease that the physician perceives. Reduction, fully or in part, of a sign or symptom may be referred to as alleviation of the sign or symptom.

In an embodiment, an effective or therapeutically effective dose of anti-TfR protein-drug conjugate is about 1 mg/kg and 50 mg/kg body weight. The dose amount may vary depending upon the age and the size of a subject to be administered, target disease, conditions, route of administration, and the like. In certain embodiments, the initial dose may be followed by administration of a second or a plurality of subsequent doses of antigen-binding protein in an amount that can be approximately the same or less or more than that of the initial dose, wherein the subsequent doses are separated by days or weeks.

In some embodiments, the anti-TfR protein-drug conjugate, or pharmaceutical compositions thereof, may be administered in accordance with a repeat dosing regimen wherein the anti-TfR protein-drug conjugate may be administered a first time (e.g., in an initial dose) and then re-administered any number of subsequent times thereafter at any amount over the time course of treatment of a subject. For example, the anti-TfR protein-drug conjugate may be re-administered once, twice, three times, four times, five times, six times, seven times, eight times, nine times, ten times, or more, over the time course of the treatment of a subject which can occur over any number of days, weeks, or years.

In some embodiments, the anti-TfR protein-drug conjugate, or pharmaceutical compositions thereof, may be administered in accordance with a stepwise dosing regimen. Stepwise dosing of a composition can refer to breaking up (i.e., dividing) dosing of the same composition over multiple administrations. In some embodiments, the dosing of the same composition is broken up once, twice, three times, four times, five times, six times, seven times, eight times, nine times, ten times, or more, over the time course of the treatment of a subject which can occur over any number of days, weeks, or years. In some embodiments, when a stepwise dose regimen is used in the administration of an anti-TfR protein-drug conjugate, the stepwise dosing regimen may result in a gradual increase in therapeutic transgene levels with each administration of the anti-TfR protein-drug conjugate.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Generation, Selection and Characterization of Immunoglobulin Molecules

Anti-human transferrin receptor (hTfR) antibodies were generated and screened for the ability to bind hTfR and for lack of strong blocking of human transferrin-hTfR binding.

Anti-hTfR Generation. VelocImmune mice were immunized with a recombinant protein comprising human transferrin receptor extracellular domain fused at N-terminus to a 6-His tag (referred to as human 6xHis-TfR) as immunogen via subcutaneous footpad injection with Alum:CpG adjuvant. Mice bleeds were collected prior to the initial immunization injection and post-boost injections, and the immune sera were subjected to antibody titer determination using a human TfR specific enzyme-linked immunosorbent assay (ELISA). In this assay serum samples in serial dilutions were added to the immunogen coated plates and plate-bound mouse IgG were detected using an HRP-conjugated anti-mouse IgG antibody. Titer of a tested serum sample is defined as the extrapolated dilution factor of the sample that produces a binding signal two times of the signal of the buffer alone control sample. The mice with optimal anti-TfR antibody titers were selected and subjected to a final boost 3-5 days prior to euthanasia and splenocytes from these mice were harvested and subject to antibody isolation using B cell sorting technology (BST).

TfR specific antibodies of isolated antibodies were isolated and characterized. Two hundred and fourteen TfR-binding antibodies were cloned into single chain fragment variables (scFvs) in complementary orientations with either the variable heavy chain followed by the variable light chain (V_{H}-V_{K}), or the variable light chain followed by the variable heavy chain (V_{K}-V_{H}), and as fragment antigen-binding regions (Fabs). Conditioned media of CHO cell culture containing the scFvs or Fabs were tested for the ability to bind hTfR proteins and hTfR-expressing cells.

### Example 2: Binding Kinetics of 32 Anti-hTfR Primary Supernatants from CHO

Biacore binding kinetics assays were conducted for the interaction of 32 anti-human TfR IgG1 monoclonal antibodies from CHO supernatants with TfR reagents at 25° C.

TABLE 2-1
Monoclonal Antibody Clones Tested

| mAb# | AbID# | Source |
|---|---|---|
| 1 | 12795B | primary supernatant |
| 2 | 12798B | primary supernatant |
| 3 | 12799B | primary supernatant |
| 4 | 12801B | primary supernatant |
| 5 | 12802B | primary supernatant |
| 6 | 12808B | primary supernatant |
| 7 | 12812B | primary supernatant |
| 8 | 12834B | primary supernatant |
| 9 | 12835B | primary supernatant |
| 10 | 12839B | primary supernatant |
| 11 | 12841B | primary supernatant |
| 12 | 12843B | primary supernatant |
| 13 | 12844B | primary supernatant |
| 14 | 12845B | primary supernatant |
| 15 | 12847B | primary supernatant |
| 16 | 12848B | primary supernatant |
| 17 | 12850B | primary supernatant |
| 18 | 31863B | primary supernatant |
| 19 | 31874B | primary supernatant |
| 20 | 12816B | primary supernatant |
| 21 | 12833B | primary supernatant |
| 22 | 69261 | primary supernatant |
| 23 | 69263 | primary supernatant |
| 24 | 69305 | primary supernatant |
| 25 | 69307 | primary supernatant |
| 26 | 69323 | primary supernatant |
| 27 | 69326 | primary supernatant |
| 28 | 69329 | primary supernatant |
| 29 | 69331 | primary supernatant |
| 30 | 69332 | primary supernatant |
| 31 | 69340 | primary supernatant |
| 32 | 69348 | primary supernatant |
| 33 | REGN1945 | |

Reagents used:
REGN2431 (hmm.hTfRC; 79210 g/mol molecular weight), having the amino acid sequence:

(SEQ ID NO: 460)
HHHHHHEQKLISEEDLGGEQKLISEEDLCKGVEPKTECERLAGTESPVRE
EPGEDEPAARRLYWDDLKRKLSEKLDSTDFTGTIKLLNENSYVPREAGSQ
KDENLALYVENQFREFKLSKVWRDQHFVKIQVKDSAQNSVIIVDKNGRLV
YLVENPGGYVAYSKAATVTGKLVHANFGTKKDFEDLYTPVNGSIVIVRAG
KITFAEKVANAESLNAIGVLIYMDQTKFPIVNAELSFFGHAHLGTGDPYT
PGFPSFNHTQFPPSRSSGLPNIPVQTISRAAAEKLFGNMEGDCPSDWKTD
STCRMVTSESKNVKLTVSNVLKEIKILNIFGVIKGFVEPDHYVVVGAQRD
AWGPGAAKSGVGTALLLKLAQMFSDMVLKDGFQPSRSIIFASWSAGDEGS
VGATEWLEGYLSSLHLKAFTYINLDKAVLGTSNFKVSASPLLYTLIEKTM
QNVKHPVTGQFLYQDSNWASKVEKLTLDNAAFPFLAYSGIPAVSFCFCED
TDYPYLGTTMDTYKELIERIPELNKVARAAAEVAGQFVIKLTHDVELNLD
YERYNSQLLSFVRDLNQYRADIKEMGLSLQWLYSARGDFFRATSRLTTDF
GNAEKTDRFVMKKLNDRVMRVEYHFLSPYVSPKESPFRHVFWGSGSHTLP
ALLENLKLRKQNNGAFNETLERNQLALATWTIQGAANALSGDVWDIDNE
F.

REGN2054 (mf TFRC ecto-mmh; 78500 g/mol molecular weight): monomeric monkey (cyno) Tfrc ectodomain (amino acids C89-F760, Accession #:XP_045243212.1) with a c-terminal myc-myc-hexahistidine tag containing a GG linker (underlined) between the 2 myc epitope sequences

| (SEQ ID NO: 461) | |
|---|---|
| | (EQKLISEEDLGGEQKLISEEDLHHHHHH). |

Equilibrium dissociation constants (K_{D}) for the interaction of anti-TfR monoclonal antibodies with human and fascicularis monkey TfR ecto domain recombinant proteins were determined using a real-time surface plasmon resonance (SPR) based Biacore S200 biosensor. All binding studies were performed in 10 mM HEPES, 150 mM NaCl, 3 mM EDTA, and 0.05% v/v surfactant Tween-20, pH 7.4 (HBS-EP) running buffer at 37° C. The Biacore CM5 sensor surface was first derivatized by amine coupling with a monoclonal mouse anti-human Fc antibody (REGN2567) followed by a step to capture anti-TfR monoclonal antibodies in CHO conditioned media. Human TfR extracellular domain expressed with a C-terminal myc-myc-hexahistidine tag (hTFR-mmh; REGN2431) or monkey TfR extracellular domain expressed with a C-terminal myc-myc-hexahistidine tag (mfTFR-mmh; REGN2054) at concentrations of 100 nM in HBS-EP running buffer were injected at a flow rate of 50 μL/min for 2 minutes. The dissociation of TfR bound to anti-TfR monoclonal antibodies was monitored for 3 minutes in HBS-EP running buffer. At the end of each cycle, the anti-TfR monoclonal antibodies capture surface was regenerated using a 12-see injection of 20 mM H₃PO₄. The association rate (ka) and dissociation rate (kd) were determined by fitting the real-time binding sensorgrams to a 1:1 binding model with mass transport limitation using Scrubber 2.0c software. The dissociation equilibrium constant (K_{D}) and dissociative half-life (t %) were calculated from the kinetic rate constants as:

$${K}_{D} ( M ) = \frac{kd}{ka} , and t 1 / 2 ( min ) = \frac{ln ( 2 )}{60 * kd}$$

The equilibrium binding constant and the kinetic binding constants are summarized in Tables 2-2 and Table 2-3 for human TfR and monkey TfR, respectively. At 25° C., anti-TfR monoclonal antibodies bound to hTfR-mmh with K_{D} values ranging from 65.6 pM to 41 nM, as shown in Table 2-2. Anti-TfR monoclonal antibodies bound to mfTfR-mmh with K_{D} values ranging from 1.16 nM to 20.5 nM, as shown in Table 2-3.

Results are set forth below.

TABLE 2-2
Equilibrium and kinetic binding parameters for the interaction of hTFR-
mmh with anti-TfR monoclonal antibodies (bivalent IgG) at 25° C.

| | mAb | 100 nM | | | | |
|---|---|---|---|---|---|---|
| | Capture | Ag Bound | kₐ | k_{d} | K_{D} | t1/2 |
| Molecule | Level (RU) | (RU) | (1/Ms) | (1/s) | (M) | (min) |
| 12795B | 3344 | 883 | 1.22E+05 | <1.00E−05 | 8.23E−11 | >1155^{#} |
| 12798B | 4552 | 1151 | 1.63E+05 | 6.33E−05 | 3.87E−10 | 182.6 |
| 12799B | 2388 | 699 | 7.76E+04 | 6.62E−05 | 8.51E−10 | 174.5 |
| 12801B | 4720 | 598 | 8.19E+04 | 4.19E−05 | 5.13E−10 | 276.0 |
| 12802B | 2828 | 903 | 1.23E+05 | 2.92E−05 | 2.36E−10 | 395.4 |
| 12808B | 4336 | 964 | 1.19E+05 | 1.07E−04 | 8.94E−10 | 108.5 |
| 12812B | 2222 | 11 | 4.33E+05 | 1.31E−03 | 3.02E−09 | 8.8 |
| 12834B | 3837 | 11 | 6.00E+05 | 4.39E−03 | 7.00E−09 | 2.6 |
| 12835B | 3276 | 1146 | 1.34E+05 | 4.17E−04 | 3.11E−09 | 27.7 |
| 12839B | 5192 | 660 | 1.18E+05 | 5.33E−05 | 4.48E−10 | 216.9 |
| 12841B | 2895 | 1151 | 1.73E+05 | 1.16E−04 | 6.70E−10 | 99.8 |
| 12843B | 3080 | 854 | 9.42E+04 | 1.68E−04 | 1.78E−09 | 68.8 |
| 12844B | 2869 | 946 | 1.31E+05 | 1.22E−04 | 9.35E−10 | 95.0 |
| 12845B | 2911 | 1104 | 1.68E+05 | 1.99E−04 | 1.18E−09 | 57.9 |
| 12847B | 4425 | 895 | 1.05E+05 | 1.71E−04 | 1.62E−09 | 67.6 |
| 12848B | 4530 | 823 | 1.01E+05 | 4.99E−05 | 4.94E−10 | 231.6 |
| 12850B | 2990 | 725 | 8.37E+04 | 1.76E−05 | 2.15E−10 | 656.8 |
| 31863B | 5490 | 1083 | 1.52E+05 | <1.00E−05 | 6.56E−11 | >1155^{#} |
| 31874B | 5594 | 904 | 1.38E+05 | 1.15E−04 | 8.36E−10 | 100.5 |
| 12816B | 3377 | 357 | 5.79E+04 | 3.36E−04 | 5.80E−09 | 34.3 |
| 12833B | 3972 | 409 | 7.20E+04 | 2.52E−04 | 3.51E−09 | 45.9 |
| 69261 | 3546 | 462 | 7.00E+04 | 8.08E−05 | 1.16E−09 | 142.9 |
| 69263 | 180 | 150 | 1.77E+05 | 1.15E−03 | 6.50E−09 | 10.0 |
| 69305 | 2726 | 6 | NB* | NB* | NB* | NB* |
| 69307 | 2349 | 1285 | 1.90E+05 | 4.26E−05 | 2.26E−10 | 271.1 |
| 69323 | 4506 | 465 | 7.34E+04 | 1.80E−04 | 2.45E−09 | 64.1 |
| 69326 | 1709 | 680 | 1.15E+05 | 2.56E−04 | 2.22E−09 | 45.1 |
| 69329 | 1573 | 1100 | 1.92E+05 | 1.86E−04 | 9.70E−10 | 62.2 |
| 69331 | 4617 | 87 | 1.14E+05 | 4.66E−03 | 4.10E−08 | 2.5 |
| 69332 | 3915 | 9 | NB* | NB* | NB* | NB* |
| 69340 | 3226 | 999 | 1.44E+05 | 6.23E−05 | 4.29E−10 | 185.4 |
| 69348 | 2848 | 680 | 1.06E+05 | 1.72E−04 | 1.62E−09 | 67.2 |
| REGN1945 | 4011 | 6 | NB | NB | NB | NB |

^{#}indicates no dissociation was observed under the current experimental conditions and the kd value was manually fixed at 1.00E−05 s⁻¹ while fitting the real time binding sensorgrams.
*NB indicates that no binding was observed under the current experimental conditions.

TABLE 2-3
Equilibrium and kinetic binding parameters for the interaction of mfTfR-
mmh with anti-TfR monoclonal antibodies (bivalent IgG) at 25° C.

| | mAb | 100 nM | | | | |
|---|---|---|---|---|---|---|
| | Capture | Ag Bound | kₐ | k_{d} | K_{D} | t1/2 |
| Molecule | Level (RU) | (RU) | (1/Ms) | (1/s) | (M) | (min) |
| 12795B | 3334 | 364 | 6.10E+04 | 7.13E−05 | 1.16E−09 | 162.0 |
| 12798B | 4542 | 508 | 7.90E+04 | 1.29E−03 | 1.63E−08 | 9.0 |
| 12799B | 2384 | 651 | 8.64E+04 | 1.62E−04 | 1.88E−09 | 71.2 |
| 12801B | 4684 | 159 | 6.40E+04 | 6.53E−04 | 1.02E−08 | 17.7 |
| 12802B | 2819 | 464 | 7.05E+04 | 5.29E−04 | 7.51E−09 | 21.8 |
| 12808B | 4329 | 626 | 7.85E+04 | 4.54E−04 | 5.78E−09 | 25.5 |
| 12812B | 2220 | 1 | NB* | NB* | NB* | NB* |
| 12834B | 3820 | 5 | NB* | NB* | NB* | NB* |
| 12835B | 3262 | 254 | 9.51E+04 | 1.68E−03 | 1.77E−08 | 6.9 |
| 12839B | 5170 | 11 | NB* | NB* | NB* | NB* |
| 12841B | 2888 | 4 | NB* | NB* | NB* | NB* |
| 12843B | 3072 | 399 | 7.90E+04 | 1.18E−03 | 1.50E−08 | 9.8 |
| 12844B | 2857 | 437 | 7.59E+04 | 7.35E−04 | 9.68E−09 | 15.7 |
| 12845B | 2906 | 13 | NB* | NB* | NB* | NB* |
| 12847B | 4420 | 702 | 9.92E+04 | 3.33E−04 | 3.36E−09 | 34.7 |
| 12848B | 4524 | 261 | 5.29E+04 | 9.62E−04 | 1.82E−08 | 12.0 |
| 12850B | 2985 | 87 | 1.01E+05 | 1.57E−03 | 1.55E−08 | 7.3 |
| 31863B | 5480 | 145 | 1.63E+05 | 2.98E−03 | 1.83E−08 | 3.9 |
| 31874B | 5557 | 26 | 6.91E+05 | 9.96E−03 | 1.44E−08 | 1.2 |
| 12816B | 3376 | 315 | 6.52E+04 | 4.85E−04 | 7.44E−09 | 23.8 |
| 12833B | 3966 | 346 | 6.92E+04 | 4.86E−04 | 7.02E−09 | 23.8 |
| 69261 | 3537 | 331 | 6.83E+04 | 4.03E−04 | 5.90E−09 | 28.7 |
| 69263 | 181 | 77 | 1.97E+05 | 3.78E−03 | 1.92E−08 | 3.1 |
| 69305 | 2725 | −7 | NB* | NB* | NB* | NB* |
| 69307 | 2344 | 3 | NB* | NB* | NB* | NB* |
| 69323 | 4500 | 24 | 5.58E+05 | 1.06E−02 | 1.90E−08 | 1.1 |
| 69326 | 1707 | 9 | NB* | NB* | NB* | NB* |
| 69329 | 1571 | 8 | NB* | NB* | NB* | NB* |
| 69331 | 4611 | 26 | 4.89E+05 | 1.00E−02 | 2.05E−08 | 1.2 |
| 69332 | 3897 | 1 | NB* | NB* | NB* | NB* |
| 69340 | 3219 | 634 | 8.92E+04 | 7.23E−04 | 8.11E−09 | 16.0 |
| 69348 | 2851 | 433 | 9.60E+04 | 4.61E−04 | 4.80E−09 | 25.1 |
| REGN1945 | 4009 | 4 | NB | NB | NB | NB |

*NB indicates that no binding was observed under the current experimental conditions.

### Example 3: Anti-TfR Antibodies Blocking Human TfRC Monomer Binding to Human Holo-Transferrin by ELISA

An ELISA-based blocking assay was developed to determine the ability of anti-Transferrin Receptor (TfR) antibodies to block the binding of human Transferrin Receptor to human holo-transferrin ligand.

TABLE 3-1
Reagents

| Reagent | Source |
|---|---|
| Human Transferrin | R&D Systems |
| polyclonal goat IgG | |
| antibody | |
| Human Holo- | R&D Systems |
| Transferrin protein | |
| His-myc-myc- | Regeneron |
| hTFRC ecto | |
| HRP conjugated c- | Novus Biologicals |
| Myc polyclonal | |
| rabbit IgG antibody | |
| 1X PBS | Irvine Scientific |
| 1X PBST (0.05% | Sigma |
| tween-20 in PBS) | |
| BSA: albumin | Sigma |
| solution from bovine | |
| serum, 30% | |
| 3-3′,5-5′- | BD Biosciences |
| tetramethylbenzidine | |
| (TMB) Substrate A | |
| 3-3′,5-5′- | BD Biosciences |
| tetramethylbenzidine | |
| (TMB) Substrate B | |
| 2N Sulfuric Acid | VWR |
| Reacti-Bind 96-well | ThermoFisher Scientific |
| plates corner notch | |
| VWR 96-Well Deep | VWR |
| Well Plates 0.5 ML | |
| Aquamax Plate | Molecular Devices |
| Washer 2000 | |
| VICTOR ™ X4 | PerkinElmer |
| Multilabel Plate | |
| Reader | |

The human Transferrin Receptor recombinant protein, hTFRC, used in the experiment was comprised of hTfR extracellular domain (amino acids C89-F760) expressed with an N-terminal 6-Histidine-myc-myc tag (Hmm.hTfrc (REGN2431): Monomeric human Tfrc ectodomain (amino acids C89-F760, Accession #: NP_001121620.1) with an N-terminal hexahistidine-myc-myc-tag containing a GG linker (underlined) between the 2 myc epitope sequences (HHHHHHEQKLISEEDLGGEQKLISEEDL) (amino acids 1-28 of SEQ ID NO: 460)). The human holo-transferrin ligand protein (holo-Tf) isolated from human plasma was purchased from R&D Systems.

In the blocking assay, the anti-human Transferrin goat IgG polyclonal antibody (anti-hTf pAb) was passively absorbed at a concentration of 2 micrograms/mL in PBS on a 96-well microtiter plate overnight at 4° C. Nonspecific binding sites were subsequently blocked using a 0.5% (w/v) solution of BSA in PBS for 1 hour at room temperature. To the same plate, human holo-Tf was then added at a concentration of 1 micrograms/mL in PBS+0.5% BSA for 2 hours at room temperature. In a separate set of 96-well microtiter plates, solutions of 300 pM Hmm-hTFRC were mixed with TFRC antibody supernatants at 2-fold dilution. After a 1-hour incubation, the mixtures were transferred to the human holo-Tf microtiter plates. After another hour incubation at room temperature, plates were washed, and plate-bound Hmm-hTFRC was detected with horseradish peroxidase (HRP) conjugated rabbit anti-Myc polyclonal antibody. The plates were developed using TMB substrate solution according to the manufacturer's recommended procedure and absorbance at 450 nm was measured on a Victor™ Multilabel Plate Reader.

Percent blocking for the tested anti-TfR antibodies was calculated using the formula below:

$$\% Blocking = 100 - [ \text{} \frac{( Test antibody - Buffer alone without Hmm - hTFRC )}{( Hmm - hTFRC alone - Buffer alone without Hmm - hTFRC )} ] \times 100$$

Antibodies that blocked binding of Hmm-hTFRC to human holo-Tf equal or more than 50% were classified as blockers.

The ability of the anti-TfR antibody to block human TFRC binding to human holo-Tf was evaluated using an ELISA-based blocking assay. In this assay, a fixed concentration of Hmm-hTFRC was pre-incubated with anti-TfR antibody containing supernatant before binding to plate immobilized human holo-Tf protein, and the plate-bound Hmm-hTFRC was detected with HRP-conjugated c-Myc specific rabbit polyclonal antibodies.

Thirty-two anti-TfR antibodies cloned into single chain fragment variables (scFvs) in complementary orientations with either the variable heavy chain followed by the variable light chain (V_{H}—VK), or the variable light chain followed by the variable heavy chain (V_{K}—V_{H}) and also as fragment antigen-binding regions (Fabs). All ninety-six anti-TfR antibody supernatants were tested for the ability to block human TFRC binding to human holo-Tf. Ninety-fouranti-TfR antibody supernatants showed no or low blocking activitywith percentage blocking ranging from 0% to 45%, and these antibodies (Fabs or scFvs formats) were classified as non-blockers (Table 3-2). Only two Fab supernatants had blocking activity greater than 50%, with % blocking values of 64% and 78% respectively.

TABLE 3-2
Summary of Anti-TfR scFv and Fab Supernatants Ability to
Block Human TFRC binding to Immobilized Human Holo-Tf

Blocking of Hmm-hTFRC Binding to
Human Holo-Tf, % Blocking

| | | scFv (V_{K}-V_{H}) | scFV (V_{H}-V_{K}) |
|---|---|---|---|
| AbPID | Fab Format | Format | Format |
| 12795B | 44 | 23 | 45 |
| 12798B | 10 | 10 | 0 |
| 12799B | 5 | 10 | 26 |
| 12801B | 45 | 27 | 37 |
| 12802B | 15 | 17 | 11 |
| 12808B | 16 | 18 | 19 |
| 12812B | 14 | 12 | 19 |
| 12816B | 14 | 14 | 16 |
| 12833B | 64 | 40 | 22 |
| 12834B | −2 | 11 | −3 |
| 12835B | 78 | 37 | 45 |
| 12839B | 13 | 6 | 23 |
| 12841B | 29 | 1 | 10 |
| 12843B | 10 | 8 | 17 |
| 12844B | 20 | 10 | 12 |
| 12845B | 11 | 3 | 18 |
| 12847B | 3 | 13 | 11 |
| 12848B | 13 | 9 | 19 |
| 12850B | 18 | 8 | 10 |
| 31863B | 24 | 7 | 13 |
| 31874B | 16 | −1 | 14 |
| 69261 | 11 | 16 | 19 |
| 69263 | 14 | 4 | 14 |
| 69305 | 3 | 5 | 3 |
| 69307 | 12 | 12 | 9 |
| 69323 | 12 | 17 | 7 |
| 69326 | −2 | 12 | 18 |
| 69329 | 8 | 19 | 25 |
| 69331 | 9 | 13 | 7 |
| 69332 | 18 | 22 | 6 |
| 69340 | 3 | 13 | 6 |
| 69348 | 40 | 16 | 0 |

### Example 4. Evaluation of Blood-Brain-Barrier Crossing of Anti-hTfR scFv Molecules

Anti-human transferrin receptor (hTfR) antibodies were generated and screened for the ability to bind hTfR and for lack of strong blocking of human transferrin-hTfR binding. Based on this initial analysis, 32 variable sequences were chosen. Domains in anti-hTfR antibodies, antigen-binding fragments (e.g., Fabs) or scFv molecules used in fusion proteins comprise the as described in Table 1-1. Sequences for the 32 anti-hTfR scFvs are disclosed in Table 1-2.

The anti-hTfR scFvs are fused to the mature peptide of human acid alpha-glucosidase (GAA). The acid alpha-glucosidase hydrolyzes alpha-1,4 linkages between the D-glucose units of glycogen, maltose, and isomaltose. The amino acid sequence for mature peptide of human alpha-glucosidase used in the fusion proteins comprises:

(SEQ ID NO: 491)
AHPGRPRAVPTQCDVPPNSRFDCAPDKAITQEQCEARGCCYIPAKQGLQG
AQMGQPWCFFPPSYPSYKLENLSSSEMGYTATLTRTTPTFFPKDILTLRL
DVMMETENRLHFTIKDPANRRYEVPLETPHVHSRAPSPLYSVEFSEEPFG
VIVRRQLDGRVLLNTTVAPLFFADQFLQLSTSLPSQYITGLAEHLSPLML
STSWTRITLWNRDLAPTPGANLYGSHPFYLALEDGGSAHGVFLLNSNAMD
VVLQPSPALSWRSTGGILDVYIFLGPEPKSVVQQYLDVVGYPFMPPYWGL
GFHLCRWGYSSTAITRQVVENMTRAHFPLDVQWNDLDYMDSRRDFTFNKD
GFRDFPAMVQELHQGGRRYMMIVDPAISSSGPAGSYRPYDEGLRRGVFIT
NETGQPLIGKVWPGSTAFPDFTNPTALAWWEDMVAEFHDQVPFDGMWIDM
NEPSNFIRGSEDGCPNNELENPPYVPGVVGGTLQAATICASSHQFLSTHY
NLHNLYGLTEAIASHRALVKARGTRPFVISRSTFAGHGRYAGHWTGDVWS
SWEQLASSVPEILQFNLLGVPLVGADVCGFLGNTSEELCVRWTQLGAFYP
FMRNHNSLLSLPQEPYSFSEPAQQAMRKALTLRYALLPHLYTLFHQAHVA
GETVARPLFLEFPKDSSTWTVDHQLLWGEALLITPVLQAGKAEVTGYFPL
GTWYDLQTVPVEALGSLPPPPAAPREPAIHSEGQWVTLPAPLDTINVHLR
AGYIIPLQGPGLTTTESRQQPMALAVALTKGGEARGELFWDDGESLEVLE
RGAYTQVIFLARNNTIVNELVRVTSEGAGLQLQKVTVLGVATAPQQVLSN
GVPVSNFTYSPDTKVLDICVSLLMGEQFLVSWC.

In order to validate the anti-human TFRC antibodies that were screened for binding in vitro, in vivo mouse studies were performed in TfrC^{hum/hum} knock-in mice to evaluate blood-brain-barrier (BBB) crossing. Eleven clones that had mature hGAA protein in brain homogenate detected by western blot were selected from this first screen of 31 antibodies.

GAA fusions by hydrodynamic delivery (HDD). Human TFRC knock-in mice were injected with DNA plasmids expressing the various anti-hTFRC antibodies in the anti-hTFRCscfv:2xG4S (SEQ ID NO: 542):hGAA format under the liver-specific mouse TTR promoter. Mice received 50 μg of DNA in 0.9% sterile saline diluted to 10% of the mouse's body weight (0.1 mL/g body weight). 48 hours post-injection, tissues were dissected from mice immediately after sacrifice by CO₂ asphyxiation, snap frozen in liquid nitrogen, and stored at −80° C.

Tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XPO4200BOX, LC2675, LC3675, LC2676). Gels were transferred to low-fluorescence polyvinylidene fluoridev (PVDF) membrane (IPFL07810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (ab137068, Abcam, Cambridge, MA, USA), or anti-GAPDH (ab9484, Abcam, Cambridge, MA, USA) and the appropriate secondary (926-32213 or 925-68070, LI-COCR, Lincoln, NE, USA). Blots were imaged with a LI-COCR Odyssey CLx.

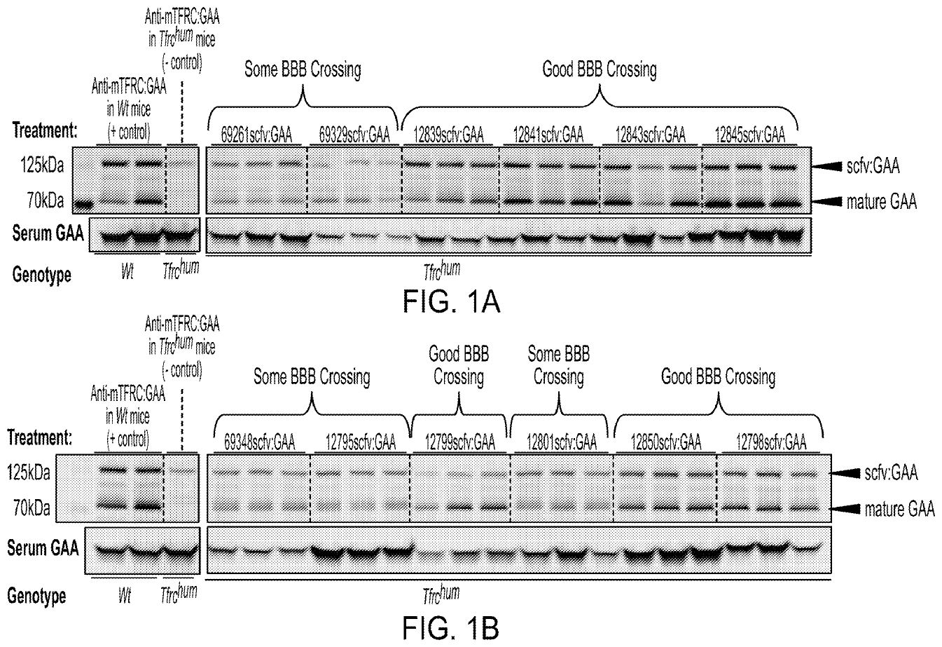

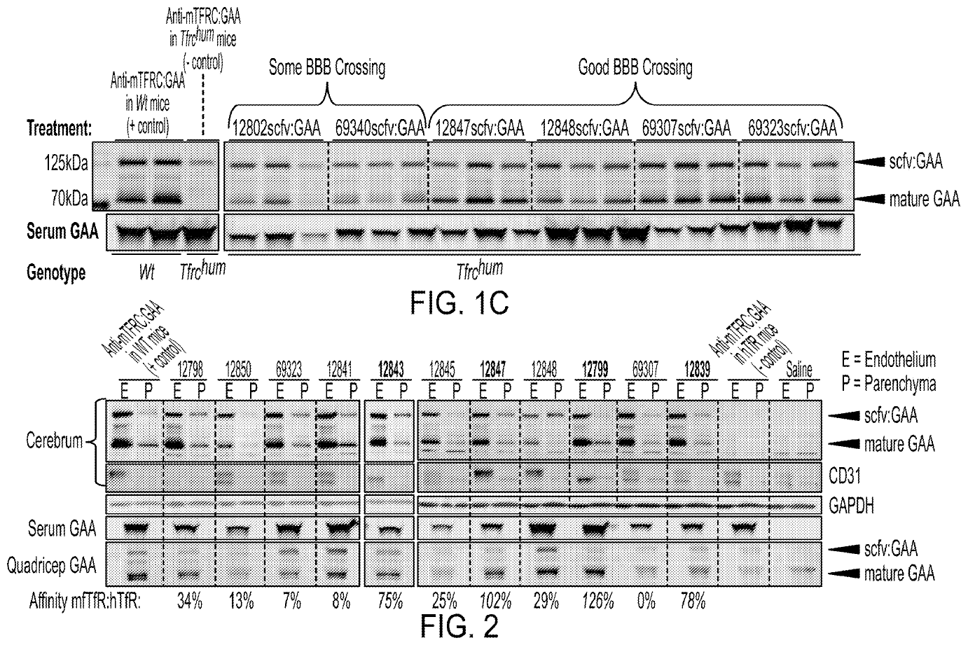

Protein band intensity was quantified in LI-COCR Image Studio software. The quantification of the mature 77 kDa GAA band for each sample was determined by first normalizing to the lane's TPS signal, then normalizing to GAA levels in the serum (loading control and liver expression control, respectively). Values were then compared to the positive control group anti-mouse TFRCscfv:hGAA in Wt mice, and negative control group anti-mTFRCscfv:hGAA in TfrcT^{hum/hum} mice (FIGS. 1A-1C, Table 4-1).

TABLE 4-1
Quantification of mature hGAA protein in brain homogenate
from mice treated HDD with anti-hTFRCscfv:hGAA plasmids.

| | | Mature hGAA protein in brain |
|---|---|---|
| Treatment group | Genotype | (normalized to positive control) |
| anti-mTFRCscfv:hGAA (positive control) | Wt | 1.00 ± 0.43* |
| anti-mTFRCscfv:hGAA (negative control) | Tfrc^{hum/hum} | 0.02 ± 0.03 |
| 69261scfv:hGAA | Tfrc^{hum/hum} | 0.67 ± 0.50 |
| 69307scfv:hGAA | Tfrc^{hum/hum} | 1.08 ± 0.19 |
| 69323scfv:hGAA | Tfrc^{hum/hum} | 0.91 ± 0.46 |
| 69329scfv:hGAA | Tfrc^{hum/hum} | 0.65 ± 0.13 |
| 69340scfv:hGAA | Tfrc^{hum/hum} | 0.55 ± 0.58 |
| 69348scfv:hGAA | Tfrc^{hum/hum} | 0.50 ± 0.05 |
| 12795scfv:hGAA | Tfrc^{hum/hum} | 0.27 ± 0.20 |
| 12798scfv:hGAA | Tfrc^{hum/hum} | 0.72 ± 0.42 |
| 12799scfv:hGAA | Tfrc^{hum/hum} | 1.05 ± 0.51* |
| 12801scfv:hGAA | Tfrc^{hum/hum} | 0.49 ± 0.18 |
| 12802scfv:hGAA | Tfrc^{hum/hum} | 0.29 ± 0.27 |
| 12839scfv:hGAA | Tfrc^{hum/hum} | 1.29 ± 0.27** |
| 12841scfv:hGAA | Tfrc^{hum/hum} | 1.72 ± 0.06*** |
| 12843scfv:hGAA | Tfrc^{hum/hum} | 1.79 ± 0.85*** |
| 12845scfv:hGAA | Tfrc^{hum/hum} | 3.08 ± 0.92*** |
| 12847scfv:hGAA | Tfrc^{hum/hum} | 1.24 ± 0.30 |
| 12848scfv:hGAA | Tfrc^{hum/hum} | 0.59 ± 0.16 |
| 12850scfv:hGAA | Tfrc^{hum/hum} | 0.47 ± 0.05 |

Data were quantified from western blot as arbitrary units (FIG. 1AC). All values are mean±SD, n=3-6 per group. One Way ANOVA vs. negative control anti-mTFRCscfv:hGAA in Tfrc^{hum/hum} mice; *p<0.05; **p<0.005; ***p<0.0001.

Capillary depletion of brain samples following HDD of anti-hTFRCscfv:hGAA plasmids. Selected anti-hTFRCscfv:hGAA from Table 4-1 were tested in a secondary screen in Tfrc^{hum} mice to determine whether hGAA was present in the brain parenchyma, and not trapped in the BBB endothelial cells. Four scFvs (12799, 12839, 12843, and 12847) were selected from this screen based on mature hGAA in the parenchyma fraction on western blot, as well as high affinity to cynomolgus TFRC.

Animals were treated HDD as detailed above. 48 hours post-injection, mice were perfused with 30 mL 0.9% saline immediately after sacrifice by CO₂ asphyxiation. A 2 mm coronal slice of cerebrum was taken between bregma and −2 mm bregma and placed in 700 μL physiological buffer (10 mM HEPES, 4 mM KCl, 2.8 mM CaCl₂, 1 mM MgSO₄, 1 mM NaH₂PO₄, 10 mM D-glucose in 0.9% saline pH 7.4) on ice. Brain slices were gently homogenized on ice with a glass dounce homogenizer. An equivalent volume of 26% dextran (MW 70,000 Da) in physiological buffer was added (final 13% dextran) and homogenized 10 more strokes. Parenchyma (supernatant) and endothelial (pellet) fractions were separated by centrifugation at 5,400 g for 15 min at 4° C. Anti-hGAA western blot was performed on fractions as detailed above (FIG. 2, Table 4-2). Blots were also probed with anti-CD31 endothelial marker (Abcam ab182982).

TABLE 4-2
Quantification of mature hGAA protein in brain parenchyma fractions and BBB
endothelial fractions of mice treated HDD with anti-hTFRCscfv:hGAA plasmids.

| | | Mature hGAA protein | Mature hGAA | |
|---|---|---|---|---|
| | | in brain parenchyma | protein in brain | Affinity to |
| | | (normalized to | endothelium | mfTFRC (% |
| | | positive | (normalized to | of hTFRC |
| Treatment group | Genotype | control) | positive control) | binding) |
| anti- | Wt | 1.00 | 5.82 | ND |
| mTFRCscfv:hGAA | | | | |
| (positive control) | | | | |
| anti- | Tfrc^{hum/hum} | 0.00 | 0.01 | ND |
| mTFRCscfv:hGAA | | | | |
| (negative control) | | | | |
| 69307scfv:hGAA | Tfrc^{hum/hum} | 1.24 | 10.73 | 0% |
| 69323scfv:hGAA | Tfrc^{hum/hum} | 0.62 | 4.18 | 7% |
| 12798scfv:hGAA | Tfrc^{hum/hum} | 0.91 | 8.37 | 34% |
| 12799scfv:hGAA | Tfrc^{hum/hum} | 0.44 | 3.99 | 126% |
| 12839scfv:hGAA | Tfrc^{hum/hum} | 0.05 | 0.84 | 78% |
| 12841scfv:hGAA | Tfrc^{hum/hum} | 0.78 | 4.23 | 8% |
| 12843scfv:hGAA | Tfrc^{hum/hum} | 1.13 | 12.99 | 75% |
| 12845scfv:hGAA | Tfrc^{hum/hum} | 2.04 | 13.06 | 25% |
| 12847scfv:hGAA | Tfrc^{hum/hum} | 0.60 | 4.96 | 102% |
| 12848scfv:hGAA | Tfrc^{hum/hum} | 0.17 | 1.24 | 29% |
| 12850scfv:hGAA | Tfrc^{hum/hum} | 0.22 | 2.25 | 13% |

hGAA protein was quantified from western blot as arbitrary units (FIG. 2). n=1 per group. Affinity to cynomolgus macaque TFRC Luminex data, calculated as percent of binding to hTFRC: (mfTFRC binding÷hTFRC binding)×100.

TABLE 4-3
Quantification of hGAA protein in quadricep of mice
treated HDD with anti-hTFRCscfv:hGAA plasmids.

| | | hGAA protein in quadricep |
|---|---|---|
| Treatment group | Genotype | (normalized to positive control) |
| Saline (vehicle) | Tfrc^{hum/hum} | 0.38 ± 0.25 |
| anti-mTFRCscfv:hGAA (positive control) | Wt | 1.07 ± 0.27 |
| anti-mTFRCscfv:hGAA (negative control) | Tfrc^{hum/hum} | 0.56 ± 0.17 |
| 69307scfv:hGAA | Tfrc^{hum/hum} | 0.58 ± 0.18 |
| 69323scfv:hGAA | Tfrc^{hum/hum} | 1.10 ± 0.19 |
| 12798scfv:hGAA | Tfrc^{hum/hum} | 1.33 ± 0.56 |
| 12799scfv:hGAA | Tfrc^{hum/hum} | 0.67 ± 0.18 |
| 12839scfv:hGAA | Tfrc^{hum/hum} | 1.80 ± 0.18 |
| 12841scfv:hGAA | Tfrc^{hum/hum} | 1.15 ± 0.12 |
| 12843scfv:hGAA | Tfrc^{hum/hum} | 1.78 ± 0.43 |
| 12845scfv:hGAA | Tfrc^{hum/hum} | 1.70 ± 1.33 |
| 12847scfv:hGAA | Tfrc^{hum/hum} | 7.74 ± 9.42 |
| 12848scfv:hGAA | Tfrc^{hum/hum} | 0.82 ± 0.18 |
| 12850scfv:hGAA | Tfrc^{hum/hum} | 0.76 ± 0.34 |

Data were quantified from western blot as arbitrary units (FIG. 2). All values are mean±SD, n=2-4 per group.

Capillary depletion of mouse brain samples following liver-depot AAV8 anti-hTFRCscfv:hGAA treatment. To confirm the HDD screen findings in a more long-term treatment model, treated Tfrc^{hum} mice were tested with selected anti-hTFRCscfv:GAA delivered as episomal liver depot AAV8 anti-hTFRCscfv:GAA under the TTR promoter. It was found that all 4 anti-hTFRCscfv:GAA delivered mature hGAA to the brain parenchyma when delivered as AAV8.

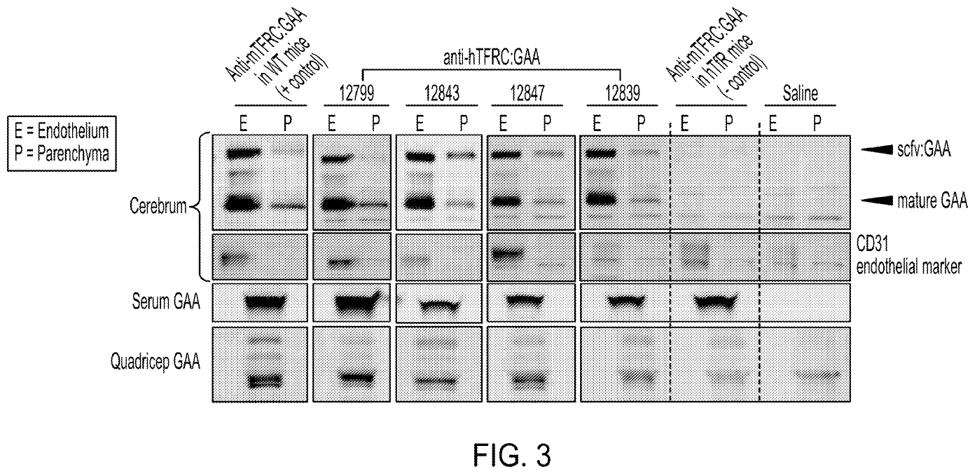

AAVproduction and in vivo transduction. Recombinant AAV8 (AAV2/8) was produced in HEK293 cells. Cells were transfected with three plasmids encoding adenovirus helper genes, AAV8 rep and cap genes, and recombinant AAV genomes containing transgenes flanked by AAV2 inverted terminal repeats (ITRs). On day 5, cells and medium were collected, centrifuged, and processed for AAV purification. Cell pellets were lysed by freeze-thaw and cleared by centrifugation. Processed cell lysates and medium were overlaid onto iodixanol gradients columns and centrifuged in an ultracentrifuge. Virus fractions were removed from the interface between the 40% and 60% iodixanol solutions and exchanged into 1×PBS with desalting columns. AAV vg were quantified by ddPCR. AAVs were diluted in PBS+0.001% F-68 Pluronic immediately prior to injection. Tfrc^{hum} mice were dosed with 3e12 vg/kg body weight in a volume of ˜100 μL. Mice were sacrificed 4 weeks post injection and capillary depletion and western blotting were performed as described above (FIG. 3, Table 4-4).

TABLE 4-4
Quantification of mature hGAA protein in brain parenchyma fractions and BBB endothelial
fractions of mice treated with liver-depot AAV8 anti-hTFRCscfv:hGAA.

| | | Mature hGAA protein in brain | Mature hGAA protein in brain |
|---|---|---|---|
| | | parenchyma (normalized to | endothelium (normalized to |
| Treatment group | Genotype | positive control) | positive control) |
| anti-mTFRCscfv:hGAA | Wt | 1.00 | 1.00 |
| (positive control) | | | |
| anti-mTFRCscfv:hGAA | Tfrc^{hum/hum} | 0.02 | 0.01 |
| (negative control) | | | |
| 12799scfv:hGAA | Tfrc^{hum/hum} | 0.94 | 0.94 |
| 12839scfv:hGAA | Tfrc^{hum/hum} | 0.49 | 0.62 |
| 12843scfv:hGAA | Tfrc^{hum/hum} | 0.61 | 0.63 |
| 12847scfv:hGAA | Tfrc^{hum/hum} | 1.90 | 1.33 |

Data were quantified from western blot as arbitrary units (FIG. 3). n=1 per group.

Rescue of glycogen storage phenotype in Gaa^{-/-}/Tfrc^{hum} mice with AAV8 episomal liver depot anti-hTFRCscfv:GAA. Three of the anti-hTFRCscfv:GAA from the above experiment were tested in Pompe disease model mice to determine whether hTFRCscfv:GAA rescued the glycogen storage phenotype. It was found that all three (12839, 12843, 12847) normalized glycogen to Wt levels.

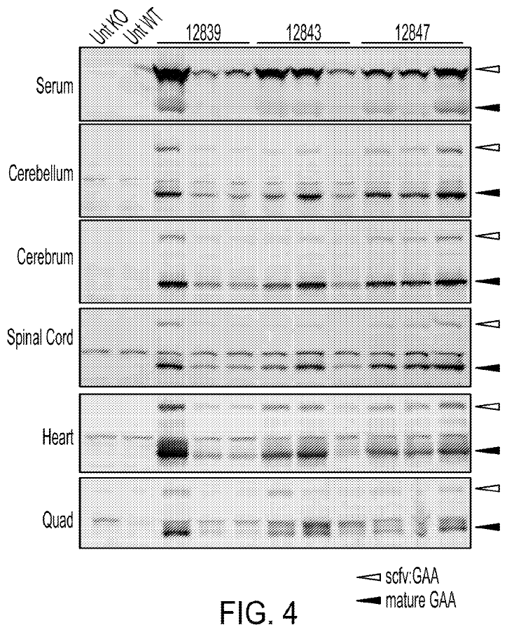

AAV production and in vivo transduction were performed as above. Gaa^{-/-}/Tfrc^{hum} mice were dosed with 2e12 vg/kg AAV8. Tissues were harvested 4 weeks post-injection and flash-frozen as above. hGAA Western blot was performed as above (FIG. 4, Table 4-5).

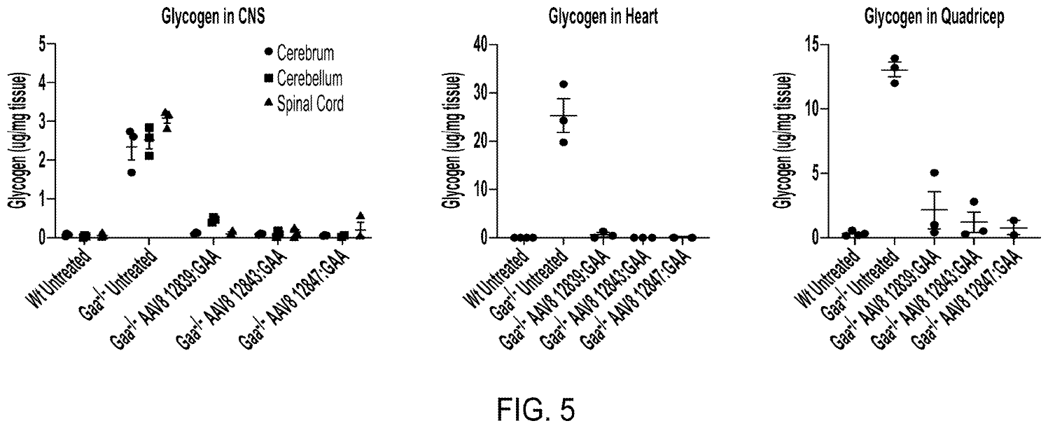

Glycogen quantification (Table 4-6, FIG. 5). Tissues were dissected from mice immediately after sacrifice by CO₂ asphyxiation, snap frozen in liquid nitrogen, and stored at −80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer's instructions (K646, BioVision, Milpitas, CA, USA).

TABLE 4-5
Quantification of hGAA protein in tissues of Gaa^{-/-}/
Tfrc^{hum} mice treated with liver-depot AAV8 anti-hTFRCscfv:hGAA.

| | | | | | | Spinal | | |
|---|---|---|---|---|---|---|---|---|
| Treatment group | n | Serum * | Liver * | Cerebrum ** | Cerebellum ** | Cord ** | Heart ** | Quadricep ** |
| Gaa^{-/-} Untreated | 1 | 0.00 | 0.02 | 0.00 | 0.00 | 0.00 | 0.02 | 0.01 |
| Gaa^{-/-} | 3 | 2.42 ± | 1.63 ± | 0.14 ± | 0.13 ± | 0.19 ± | 0.53 ± | 0.14 ± |
| 12839scfv:hGAA | | 2.41 | 0.96 | 0.12 | 0.12 | 0.19 | 0.52 | 0.16 |
| Gaa^{-/-} | 3 | 2.07 ± | 2.23 ± | 0.17 ± | 0.11 ± | 0.17 ± | 0.49 ± | 0.18 ± |
| 12843scfv:hGAA | | 1.35 | 0.08 | 0.07 | 0.05 | 0.09 | 0.31 | 0.06 |
| Gaa^{-/-} | 3 | 1.56 ± | 1.40 ± | 0.25 ± | 0.21 ± | 0.42 ± | 0.58 ± | 0.19 ± |
| 12847scfv:hGAA | | 0.71 | 0.13 | 0.04 | 0.09 | 0.19 | 0.17 | 0.08 |

Data were quantified from western blot as arbitrary units (FIG. 4). All values are mean ± SD, n = 1-3 per group.
* Total hGAA protein;
** Mature hGAA protein.

TABLE 4-6
Quantification of glycogen in tissues of Gaa^{—/—}/Tfrc^{hum}
mice treated with liver-depot AAV8 anti-hTFRCscfv:hGAA.

| Treatment group | Cerebrum | Cerebellum | Spinal Cord | Heart | Quadricep |
|---|---|---|---|---|---|
| Wt Untreated | 0.06 ± 0.04* | 0.01 ± 0.04* | 0.05 ± 0.05* | 0.08 ± 0.02* | 0.34 ± 0.19* |
| Gaa—/— Untreated | 2.34 ± 0.58 | 2.51 ± 0.38 | 3.08 ± 0.23 | 25.30 ± 6.06 | 13.05 ± 0.98 |
| Gaa—/— 12839scfv:hGAA | 0.11 ± 0.03* | 0.46 ± 0.08* | 0.08 ± 0.10* | 0.68 ± 0.68* | 2.15 ± 2.52* |
| Gaa—/— 12843scfv:hGAA | 0.09 ± 0.02* | 0.09 ± 0.08* | 0.13 ± 0.13* | 0.09 ± 0.01* | 1.22 ± 1.39* |
| Gaa—/— 12847scfv:hGAA | 0.05 ± 0.01* | 0.02 ± 0.03* | 0.20 ± 0.33* | 0.11 ± 0.11* | 0.80 ± 0.79* |

Rescue of glycogen storage in brain and muscle in Gaa^{-/-}/Tfrc^{hum} mice with AAV8 episomal liver depot anti-hTFRCscfv:GAA. Three selected anti-hTFRCscfv:GAA (12799, 12843, and 12847) were tested in Pompe disease model mice to determine whether hTFRCscfv:GAA rescued the glycogen storage phenotype. In this experiment, histology was performed on brain and muscle sections to visualize glycogen in the tissues. It was found that all three selected anti-hTFRCscfv:GAA reduced glycogen staining in the brain and muscle. 12847scfv:GAA was selected for further analysis based on these data.

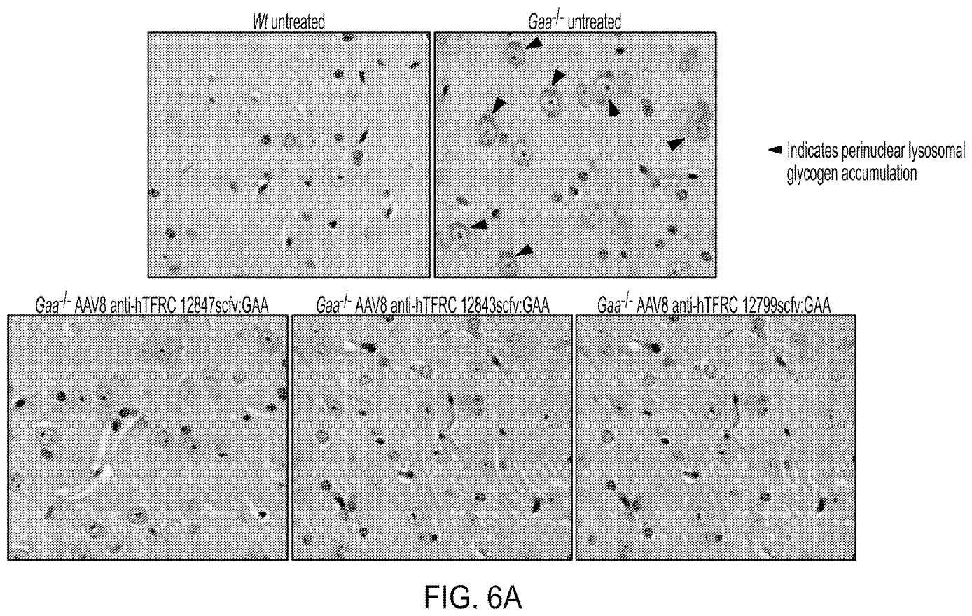

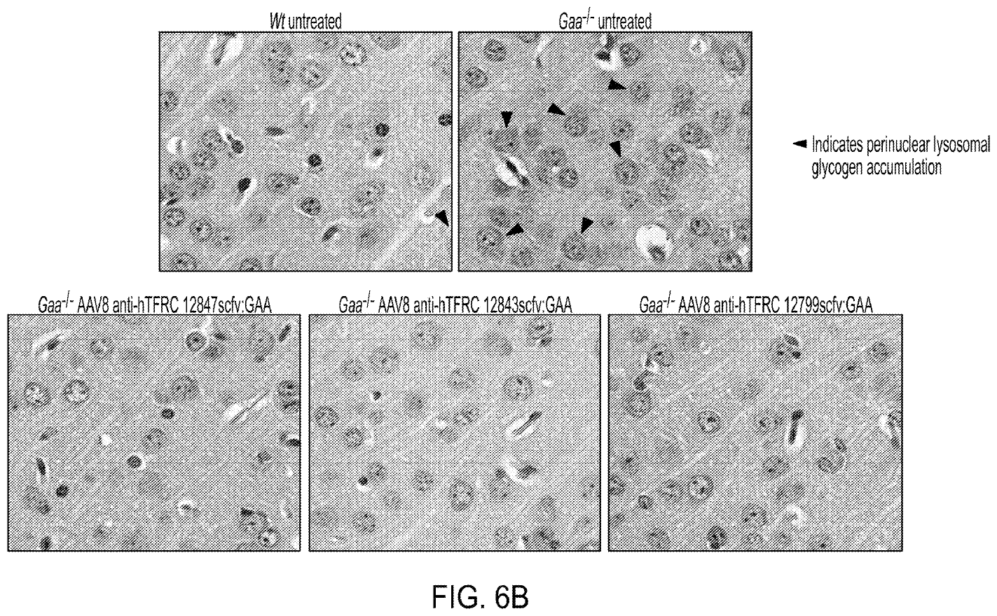

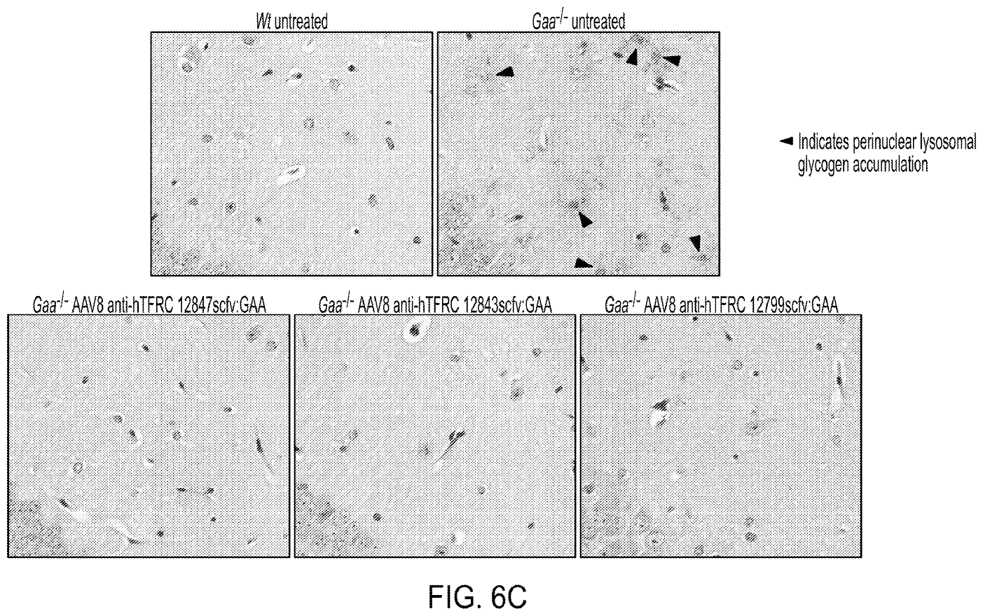

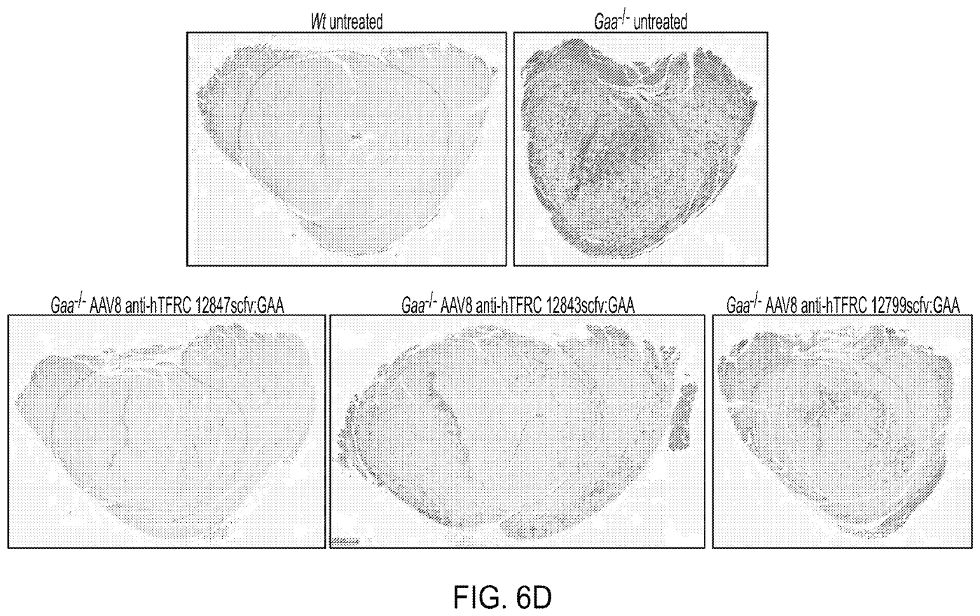

AAV production and in vivo transduction were performed as above. Three-month old Gaa^{-/-}/Tfrc^{hum} mice were dosed with 4e11 vg/kg AAV8. 4 weeks post-injection, tissues were frozen for glycogen analysis as above (Table 4-7). For histology, animals were perfused with saline (0.9% NaCl), and tissues were drop-fixed overnight in 10% Normal Buffered Formalin. Tissues were washed 3× in PBS and stored in PBS/0.01% sodium azide until embedding. Tissues were embedded in paraffin and 5 um sections were cut from brain (coronal, −2 mm bregma) and quadricep (fiber cross-section). Sections were stained with Periodic Acid-Schiff and Hematoxylin using standard protocols (FIGS. 6A-6D).

TABLE 4-7
Quantification of glycogen in tissues of Gaa^{—/—}/Tfrc^{hum}
mice treated with liver-depot AAV8 anti-hTFRCscfv:hGAA.

| Treatment group | Cerebellum | Quadricep |
|---|---|---|
| Wt Untreated | 0.02 ± 0.03* | 0.55 ± 0.10* |
| Gaa—/— Untreated | 1.91 ± 0.26 | 12.19 ± 3.02 |
| Gaa—/— 12799scfv:hGAA | 0.10 ± 0.06* | 1.34 ± 0.9* |
| Gaa—/— 12843scfv:hGAA | 0.09 ± 0.06* | 1.09 ± 1.27* |
| Gaa—/— 12847scfv:hGAA | 0.07 ± 0.06* | 0.72 ± 0.64* |

All values are glycogen μg/mg tissue, mean ± SD, n = 5-8 per group. One Way ANOVA *p < 0.0001 vs. Gaa^{-/-} Untreated group.

Insertion of anti-hTFRC 12847scfv:GAA in Gaa^{-/-}/Tfrc^{hum} mice. The selected anti-hTFRC 12847scfv:GAA was tested in Pompe disease model mice by albumin insertion to determine whether the results can be replicated with episomal AAV8 liver depot expression. Albumin insertion of 12847scfv:GAA delivered mature hGAA protein to the brain and muscle, and rescued the glycogen storage phenotype in Gaa^{-/-}/Tfrc^{hum} mice. These data were produced with the native 12847scfv:GAA sequence that is not optimized.

12847scfv:GAA was compared to the muscle-targeted anti-hCD63scfv:GAA in Gaa^{-/-}/Cd63^{hum} mice. In this particular experiment, the expression of anti-hCD63scfv:GAA was lower than usual and does not deliver as much GAA protein to the muscle nor normalize glycogen as it usually does. This may make it appear that anti-hCD63scfv:GAA is less effective than 12847scfv:GAA in the muscle but in most experiments, they were found to be comparable in the muscle.

AA Vproduction. A promoterless AAV genome plasmid was created with the 12847scfv:GAA sequence and the mouse albumin exon 1 splice acceptor site at the 3′ end. Recombinant AAV8 (AAV2/8) was produced in HEK293 cells. Cells were transfected with three plasmids encoding adenovirus helper genes, AAV8 rep and cap genes, and recombinant AAV genomes containing transgenes flanked by AAV2 inverted terminal repeats (ITRs). On day 5, cells and medium were collected, centrifuged, and processed for AAV purification. Cell pellets were lysed by freeze-thaw and cleared by centrifugation. Processed cell lysates and medium were overlaid onto iodixanol gradients columns and centrifuged in an ultracentrifuge. Virus fractions were removed from the interface between the 40% and 60% iodixanol solutions and exchanged into 1×PBS with desalting columns. AAV vg were quantified by ddPCR.

In vivo CRISPR/Cas9 insertion into the albumin locus. 3-month old Gaa^{-/-}/Tfrc^{hum} mice were dosed via tail vein injection with 3e12 vg/kg AAV8 12847scfv:GAA and 3 mg/kg LNP gRNA/Cas9 mRNA diluted in PBS+0.001% F-68 Pluronic. Mice were sacrificed 3 weeks post injection. Negative control mice received insertion AAV8 without LNP. Positive control mice were dosed with 4e11 vg/kg episomal liver depot AAV8 12847scfv:GAA under the TTR promoter (phenotype rescue data previously shown). Tissues were dissected from mice immediately after sacrifice by CO₂ asphyxiation, snap frozen in liquid nitrogen, and stored at −80° C. Blood was collected from mice by cardiac puncture immediately following CO₂ asphyxiation and serum was separated using serum separator tubes (BD Biosciences, 365967).

TABLE 4-8
Treatment Groups and Controls.

| Treatment group | Genotype | Function |
|---|---|---|
| Wt Untreated | Tfrc^{hum} | Normal untreated mouse control |
| Gaa^{—/—} untreated | Gaa^{—/—}/Tfrc^{hum} | Untreated Pompe disease mouse |
| Gaa^{—/—} insertion AAV only | Gaa^{—/—}/Tfrc^{hum} | Negative control for insertion (no Cas9/gRNA delivered) |
| Gaa^{—/—} episomal AAV8 | Gaa^{—/—}/Tfrc^{hum} | Positive control, previously shown rescue of glycogen |
| TTR 12847scfv:hGAA | | storage phenotype |
| Gaa^{—/—} insertion | Gaa^{—/—}/Tfrc^{hum} | Experimental insertion group |
| 12847scfv:hGAA | | |
| Gaa^{—/—} untreated | Gaa^{—/—}/Cd63^{hum} | Untreated Pompe disease mouse (CD63 humanized) |
| Gaa^{—/—} insertion anti- | Gaa^{—/—}/Cd63^{hum} | Negative control for BBB-crossing (muscle targeted) |
| CD63scfv:hGAA | | |

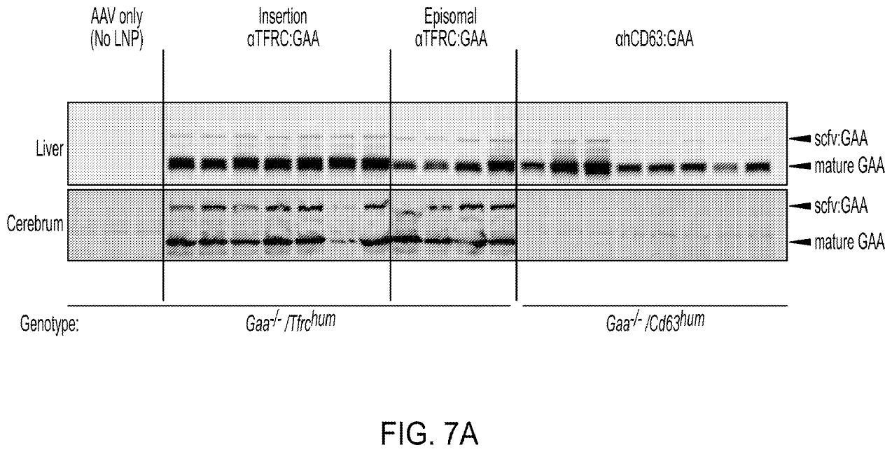

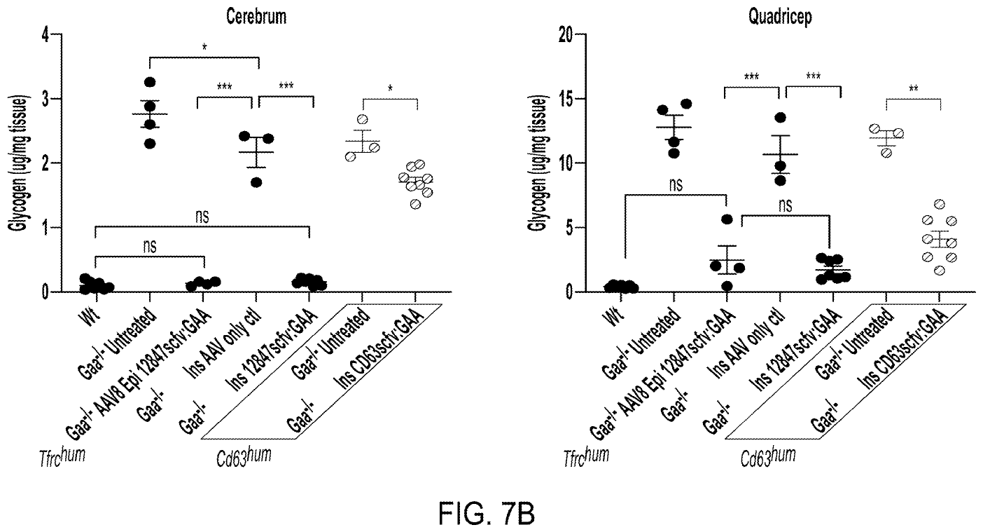

Western blot (Table 4-9, FIG. 7A). Tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XPO4200BOX, LC2675, LC3675, LC2676). Gels were transferred to low-fluorescence polyvinylidene fluoridev (PVDF) membrane (IPFL07810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (abl37068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx.

Protein band intensity was quantified in LI-COR Image Studio software. The quantification of the mature 77 kDa GAA band for each sample was determined by normalizing to the lane's TPS signal (loading control).

Glycogen quantification (Table 4-10, FIG. 7B). Tissues were dissected from mice immediately after sacrifice by CO₂ asphyxiation, snap frozen in liquid nitrogen, and stored at −80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer's instructions (K646, BioVision, Milpitas, CA, USA).

TABLE 4-9
Quantification of hGAA protein in tissues of Gaa^{—/—}/Tfrc^{hum}
mice treated with insertion anti-hTFRC 12847scfv:hGAA.

| | | | Cerebrum | Quadricep |
|---|---|---|---|---|
| | Liver | Serum | mature | mature |
| Treatment group | total hGAA | total hGAA | hGAA | hGAA |
| Gaa^{—/—} insertion AAV only negative control | 0.02 ± 0.003 | 0.03 ± 0.02 | 0.002 ± 0.001 | 0.006 ± 0.002 |
| Gaa^{—/—} episomal AAV8 TTR 12847scfv:hGAA | 2.35 ± 0.72 | 3.65 ± 2.09 | 0.49 ± 0.20^{§§} | 0.148 ± 0.043^{§§} |
| Gaa^{—/—} insertion 12847scfv:hGAA | 4.31 ± 0.87* | 3.47 ± 2.37 | 0.57 ± 0.26^{§§} | 0.141 ± 0.062^{§§} |
| Gaa^{—/—} insertion anti-CD63scfv:hGAA | 2.67 ± 1.04* | 0.93 ± 0.55* | 0.01 ± 0.003 | 0.060 ± 0.037 |

All values are glycogen μg/mg tissue, mean ± SD, n = 5-8 per group. One Way ANOVA *p < 0.005 vs. Gaa^{-/-} episomal AAV8 TTR 12847scfv:GAA group; ^{§§}p < 0.001 vs. AAV only negative control group.

TABLE 4-10
Quantification of glycogen in tissues of Gaa^{—/—}/Tfrc^{hum}
mice treated with insertion anti-hTFRC 12847scfv:hGAA.

| Treatment group | Cerebrum | Quadricep |
|---|---|---|
| Wt untreated | 0.10 ± 0.07 | 0.37 ± 0.13 |
| Gaa^{—/—}/Tfrc^{hum} untreated (Tfrc^{hum}) | 2.76 ± 0.41 | 12.75 ± 1.88 |
| Gaa^{—/—}/Tfrc^{hum} insertion AAV only | 2.17 ± 0.40* | 10.64 ± 2.56 |
| Gaa^{—/—}/Tfrc^{hum} episomal AAV8 TTR 12847scfv:hGAA | 0.13 ± 0.03***^{§} | 2.44 ± 2.21***^{§} |
| Gaa^{—/—}/Tfrc^{hum} insertion 12847scfv:hGAA | 0.16 ± 0.05***^{§} | 1.67 ± 0.76***^{§} |
| Gaa^{—/—}/Cd63^{hum} untreated | 2.34 ± 0.30 | 11.91 ± 1.01 |
| Gaa^{—/—}/Cd63^{hum} insertion anti-CD63scfv:hGAA | 1.71 ± 0.20* | 4.06 ± 0.13** |

All values are glycogen μg/mg tissue, mean ± SD, n = 5-8 per group. One Way ANOVA *p < 0.005 vs. Gaa^{-/-} /Cd63^{hum} untreated group; **p < 0.001 vs. Gaa^{-/-}/Cd63^{hum} unrteated group;
***p =21 0.0001 vs. Gaa^{-/-}Tfrc^{hum} untreated group; ^{§}non-significant vs. Wt untreated group.

To assess whether glycogen reduction translates into improved muscle function, the mice are tested on grip strength apparatuses at a time point post-administration. Limb grip strength is measured with a force meter (Columbus Instruments, Columbus, OH, USA). All tests are performed in triplicate.

In summary, the anti-TfR:GAA protein expressed from hepatocytes are successfully delivered to muscle cells and CNS cells in the mice, demonstrating superior muscle targeting ability and blood-brain barrier crossing ability of the anti-TfR scFvs.

### Example 5. MRNA Knockdown in Target Tissues Following Systemic Delivery of Antibody-Oligonucleotide Conjugate (AOC)

To enhance muscle- and/or brain-specific delivery (e.g., muscle- and/or brain-specific delivery) delivery of therapeutic oligonucleotides, antibodies targeting human transferrin receptor (hTfR), are developed. Conjugation of oligonucleotides (e.g., siRNA, ASO) to these anti-hTfR antibodies, antibody variants, and/or antibody fragments, can provide efficient gene knockdown or exon skipping in various tissues such as for the treatment of various diseases or disorders, e.g., muscular and/or neurological disease(s) or disorder(s) described herein. Table 5-1 shows a description of anti-hTfR antibody siRNA conjugates for testing in the present Example. It should be noted that any of various anti-hTfR antibodies, or variants, or fragments thereof disclosed herein (exemplified as “REGN hTfR Ab1” and “REGN hTfR Ab2” in the Table below) may be tested. The control siRNA conjugates listed in the table below are merely non-limiting examples of such controls.

TABLE 5-1
Anti-hTfR antibody siRNA conjugates for testing

| REGN ID | Description |
|---|---|
| REGN hTfR Ab1-M3463- | hTfR hlgG1 antibody 1 conjugated with siRNA |
| siRNA1 | |
| REGN hTfR Ab2-M3463- | hTfR hlgG1 antibody 2 conjugated with siRNA |
| siRNA1 | |
| Isotype control Ab-M3463- | Isotype control hlgG1 antibody conjugated with siRNA |
| siRNA1 | |
| Control Ab1-M3463-siRNA1 | Isotype control rlgG2a antibody conjugated with siRNA |
| Control Ab2-M3463-siRNA1 | rlgG2a antibody targeting a different target conjugated |
| | with siRNA |

Table 5-2 provides specific non-limiting examples of anti-hTfR antibody siRNA conjugates which may be tested in accordance with the methods set forth herein.

TABLE 5-2
Non-limiting examples of anti-hTfR antibody siRNA conjugates
Conjugates

REGN17514 (H1H12847B)-M3463-siRNA1
REGN17512 (H1H12843B)-M3463-siRNA1
REGN17510 (H1H12839B)-M3463-siRNA1
REGN17508 (H1H12799B)-M3463-siRNA1
REGN17513 (H1H12845B)-M3463-siRNA1
REGN17708 (H1H12812B)-M3463-siRNA1

M3463 is an example linker that can be used in the antibody siRNA conjugates described herein. The structure of M3463 is shown below (purchased from Broadpharm, BP-22617).

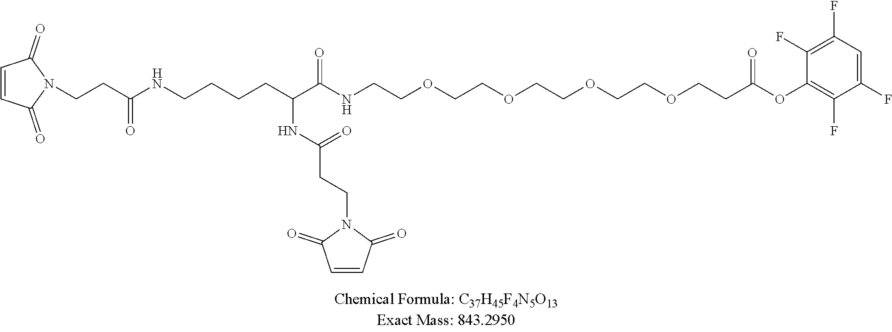 [SMILES: O=C(CCN1C(=O)C=CC1=O)NCCCCC(NC(=O)CCN1C(=O)C=CC1=O)C(=O)NCCOCCOCCOCCOCCC(=O)Oc1c(F)c(F)cc(F)c1F]

Anti-hTfR antibody is conjugated with siRNA against any one of various targets described herein, e.g., DMPK, CNBP, Dystrophin, DUX4, ApoE, MAPT, APP, HTT, SOD1, C9orf72, SNCA, LRRK2, PRNP, SLC5A1, SLC16A3, HDAC6, MMP27, MFAP5, FAM64A, BAIAP3, MYH7, TPM1, RBM20, KLHL24, MYL2, or TNNT2 gene or a mutant thereof, to test antibody-oligonucleotide conjugate-targeted knockdown of the target gene or silencing of the disease-specific allele in a specific tissue, e.g., muscle, heart and/or brain, in mice.

The conjugation reaction consists of 2 steps: For the first step, a transglutaminase enzyme is used to add N3 (azido group) to the antibody. For the transglutaminase enzyme to work, sugar residues at Asparagine (N297) are removed by changing N297 to aspartate (D). For the second step, click chemistry is used to conjugate siRNA.

The siRNA against the target gene (siRNA1) comprises the nucleotide sequence that targets the target gene. The control reagent is a non-targeting antibody conjugated with the same siRNA, and the comparator molecules is an antibody against a different target and its associated isotype control conjugated with siRNA. Seven-week-old C57BL6 male mice are maintained on chow diet and are tail vein injected with PBS, or about 0.1 to about 5.0 mg/kg of the antibody siRNA conjugates of Table 5-1. Five mice per group are used for experiments. Bleeds for detection of circulating antibody are performed at baseline (1 week before dosing), 2 hours, 1 day, 4 days and 7 days post-dosing.

Seven days after injection, mice are anesthetized by isoflurane and then sacrificed by cervical dislocation. Gastrocnemius, tibialis anterior, soleus, quadriceps, diaphragm, brain, spinal cord, retina, and peripheral nerve (e.g., femoral nerve) are collected in RNAlater, as well as the heart, liver, spleen, kidney, and lungs. After resting in RNAlater for 4 hours, samples are frozen at −20° C. until RNA isolation.

Fc ELISAs—To determine circulating antibody levels, high binding 96-well clear plates (Thermo Scientific Pierce, catalog #15041) are coated overnight with antibodies against human and rat IgG (Jackson Immunoresearch, Catalog #109-005-098, 112-005-167). Plates are washed, blocked, washed again, and then incubated with diluted serum samples from bleeds described before. Plates are washed again and incubated with antibodies against human and rat IgG conjugated with horseradish peroxidase. Plates are washed and incubated with TMB substrate followed by sulfuric acid to stop the reaction, and then are read on an optical plate reader at 450 nm wavelength.

RNA isolation—RNA is purified from samples in RNAlater using the automated MagMax (Thermo Scientific) protocol.

cDNA synthesis/qPCR—250 ng-1 μg of RNA sample is treated with DNAse I (Thermo Scientific Catalog #EN0521) and then reverse transcribed using the SuperScript VILO Master Mix (Invitrogen Catalog #11755-050). cDNA is diluted 10-fold with nuclease-free H₂O, and then assayed by qPCR. TaqMan assays are used to determine expression of the target gene with the TaqMan Gene Expression Master Mix (Applied Biosystems, Catalog #4369106) on the QuantStudio 6 Flex System (Applied Biosystems).

### Example 6. Epitope Mapping for Transferrin (TfR) Antibodies

Hydrogen-Deuterium Exchange Mass Spectrometry (HDX-MS) was performed to delineate regions in mouse and human Transferrin (m/hTfR) involved in binding of anti-Transferrin Receptor (TfR) antibodies. The anti-TfR monoclonal antibodies tested are described in Table 6-1. The reagents used and corresponding lot numbers are set forth in Table 6-2.

TABLE 6-1
Monoclonal Antibody Clones Tested

| REGN# | AbPID | Lot # |
|---|---|---|
| REGN17507 | H1H12798B | L1 |
| REGN17508 | H1H12799B | L1 |
| REGN17509 | H1H12835B | L1 |
| REGN17510 | H1H12839B | L1 |
| REGN17511 | H1H12841B | L1 |
| REGN17512 | H1H12843B | L1 |
| REGN17513 | H1H12845B | L1 |
| REGN17514 | H1H12847B | L1 |
| REGN17515 | H1H12848B | L1 |
| REGN17516 | H1H12850B | L1 |
| REGN17517 | H1H31874B | L1 |

TABLE 6-2
Reagents Used and Lot Numbers

| REGN# | Lot # | Description |
|---|---|---|
| REGN2120 | 03-121015 | hTfR(C89-F763).mmh |
| REGN2431 | L1 | hmm.hTfR(C89-F763) |

A general description of the HDX-MS method is set forth in, e.g., Ehring (1999) Analytical Biochemistry 267(2):252-259; and Engen and Smith (2001) Anal. Chem. 73:256A-265A. The experiment was performed on a customized HDX automation system (NovaBioAssays, MA) coupled to a Q Exactive HF mass spectrometer (Thermo Fisher Scientific, MA).

PBS-D₂O buffer was prepared by dissolving one PBS tablet in 100 mL 99.9% D₂O to form solution of 10 mM sodium phosphate, 137 mM NaCl, 3 mM KCl, pD 7.0 (equivalent to pH 7.4 at 25° C.). To initiate deuterium exchange, 10 μL of protein sample (hTfR alone, or hTfR in mixture with either of the monoclonal mAbs listed above, see, e.g., Table 6-1) was diluted with 90 μL PBS-D₂O buffer. After 5 minutes or 10 minutes, deuterium exchange was quenched by adding 100 μL quenching buffer (0.5 M TCEP, 4 M guanidine hydrochloride, pH 2.08) followed by 90 second incubation at 20° C. The quenched samples were digested by online pepsin/protease XIII column (NovaBioAssays, MA) at room temperature with 100 μL/min 0.1% formic acid in water. Peptic peptides were trapped by an ACQUITY UPLC Peptide BEH C18 VanGuard Pre-column (2.1×5 mm, Waters, MA) and further separated by an ACQUITY UPLC Peptide BEH C18 column (2.1 x 50 mm, Waters, MA) at −5° C., using 10-minute or 15-minute gradients with 0.1% formic acid in water and 0.1% formic acid in acetonitrile as mobile phases at 200 μL/min. Eluted peptides were analyzed by the mass spectrometer in LC-MS/MS or LC-MS mode.

A set of non-deuterated samples was prepared in PBS—H₂O buffer and analyzed with the method described above to identify peptide sequences and determine peptide masses without deuterium exchange. The LC-MS/MS data of non-deuterated samples were searched against a database containing sequences of hTfR, pepsin and protease XIII using the Byonic search engine (Protein Metrics, CA) with parameters for non-specific enzymatic digestion. The identified peptide list was then imported into the HDExaminer software (Sierra Analytics, CA) together with LC-MS data from all deuterated samples to calculate the deuterium uptake percentage (D %) of individual peptides from hTfR. Differences in deuterium uptake were calculated as ΔD %=D % of hTfR-mAb−D % of hTfR. Differences were considered significant if ΔD %<−5% (equivalent to |AD|>5% and ΔD %<0, averaged from 2 replicates). Mass spectra of peptides showing significant differences were examined manually to ensure that correct isotopic patterns were used for D % calculations by the software.

Two TfR protein constructs were used in HDX-MS experiments by reason of reagent availability and antibody specificity: hTfR(C89-F763).mmh, and hmm.hTfR(C89-F763). HDX data were obtained on 88%-95% of amino acids in hTfR with mmh tag. The numerical range provided before each amino acid sequence in the list below indicates the amino acid (aa) residue positions in hTfR which are protected by the indicated antibody. These amino acid residue positions are indicative of antibody binding sites on hTfR and does not provide residue-level contacts between them. Due to the nature of HDX-MS technique, the regions obtained by HDX-MS may be larger or smaller than actual contacts determined by high-resolution structural studies such as X-ray crystallography and cryogenic electron microscopy methods.

REGN17507 (H1H12798B) protects the following regions in hTfR:

| | 146-167 |
|---|---|
| (SEQ ID NO: 507) | |
| | LLNENSYVPREAGSQKDENLAL; |
| | 281-295 |
| (SEQ ID NO: 508) | |
| | IYMDQTKFPIVNAEL; |
| | and |
| | 572-576 |
| (SEQ ID NO: 509) | |
| | TYKEL. |

REGN17508 (H1H12799B) protects the following regions in hTfR:

| | 128-146 |
|---|---|
| (SEQ ID NO: 510) | |
| | KRKLSEKLDSTDFTGTIKL; |
| | 503-522 |
| (SEQ ID NO: 511) | |
| | YTLIEKTMQNVKHPVTGQFL; |
| | and |
| | 576-592 |
| (SEQ ID NO: 512) | |
| | LIERIPELNKVARAAAE. |

REGN17509 (H1H12835B) protects the following region in hTfR:

147-165
(SEQ ID NO: 513)
LNENSYVPREAGSQKDENL.

REGN17510 (H1H12839B) protects the following region in hTfR:

238-246
(SEQ ID NO: 514)
GTKKDFEDL.

REGN17511 (H1H12841B) protects the following region in hTfR:

199-224
(SEQ ID NO: 515)
SVIIVDKNGRLVYLVENPGGYVAYSK.

REGN17512 (H1H12843B) protects the following regions in hTfR:

146-164
(SEQ ID NO: 516)
LLNENSYVPREAGSQKDEN;
284-295
(SEQ ID NO: 517)
DQTKFPIVNAEL;
and
572-585
(SEQ ID NO: 518)
TYKELIERIPELNK.

REGN17513 (H1H12845B) protects the following region in hTfR:

199-222
(SEQ ID NO: 519)
SVIIVDKNGRLVYLVENPGGYVAY.

REGN17514 (H1H12847B) protects the following regions in hTfR:

146-164
(SEQ ID NO: 516)
LLNENSYVPREAGSQKDEN;
and
572-585
(SEQ ID NO: 518)
TYKELIERIPELNK.

REGN17515 (H1H12848B) protects the following regions in hTfR:

281-295
(SEQ ID NO: 508)
IYMDQTKFPIVNAEL;
and
346-365
(SEQ ID NO: 520)
FGNMEGDCPSDWKTDSTCRM.

REGN17516 (H1H12850B) protects the following regions in hTfR:

146-167
(SEQ ID NO: 507)
LLNENSYVPREAGSQKDENLAL;
212-232
(SEQ ID NO: 522)
LVENPGGYVAYSKAATVTGKL;
281-297
(SEQ ID NO: 523)
IYMDQTKFPIVNAELSF;
337-345
(SEQ ID NO: 524)
ISRAAAEKL;
366-383
(SEQ ID NO: 525)
VTSESKNVKLTVSNVLKE;
and
557-572
(SEQ ID NO: 526)
FCEDTDYPYLGTTMDT

REGN17517 (H1H31874B) protects the following region in hTfR:

243-246
(SEQ ID NO: 521)
FEDL.

The minimal amino acid sequence in hTfR which is protected by the above-listed anti-TfR antibodies (i.e., the minimal epitope sequence), numerical range indicating the amino acid (aa) residue positions in hTfR which are protected each antibody, as well as the conformational or linear nature of each minimal epitope are described in Table 6-3. Each of the minimal epitopes is bound by its corresponding antibody at one or more amino acid residues within the minimal epitope sequence.

TABLE 6-3
Minimal epitope sequences in hTfR protected by anti-TfR antibodies

| | | | Amino | | |
|---|---|---|---|---|---|
| | | | acid | | |
| | Epitope | | residue | Amino acid | SEQ |
| Antibody ID | No. | Class | positions | sequence | ID NO |
| REGN17507 | 1 | conformational | 146-149 | LLNE | 529 |
| (H1H12798B) | | | | | |
| REGN17507 | 2 | conformational | 572-576 | TYKEL | 509 |
| (H1H12798B) | | | | | |
| REGN17508 | 1 | conformational | 136-143 | DSTDFTGT | 530 |
| (H1H12799B) | | | | | |
| REGN17508 | 2 | conformational | 513-521 | VKHPVTGQF | 531 |
| (H1H12799B) | | | | | |
| REGN17508 | 3 | conformational | 577-583 | IERIPEL | 532 |
| (H1H12799B) | | | | | |
| REGN17509 | 1 | linear | 147-164 | LNENSYVPREAGSQKDEN | 533 |
| (H1H12835B) | | | | | |
| REGN17510 | 1 | linear | 243-246 | FEDL | 521 |
| (H1H12839B) | | | | | |
| REGN17511 | 1 | linear | 202-209 | IVDKNGRL | 534 |
| (H1H12841B) | | | | | |
| REGN17512 | 1 | conformational | 146-149 | LLNE | 529 |
| (H1H12843B) | | | | | |
| REGN17512 | 2 | conformational | 572-576 | TYKEL | 509 |
| (H1H12843B) | | | | | |
| REGN17513 | 1 | linear | 202-211 | IVDKNGRLVY | 535 |
| (H1H12845B) | | | | | |
| REGN17514 | 1 | conformational | 146-149 | LLNE | 529 |
| (H1H12847B) | | | | | |
| REGN17514 | 2 | conformational | 572-576 | TYKEL | 509 |
| (H1H12847B) | | | | | |
| REGN17515 | 1 | linear | 284-288 | DQTKF | 536 |
| (H1H12848B) | | | | | |
| REGN17516 | 1 | conformational | 212-218 | LVENPGGY | 537 |
| (H1H12850B) | | | | | |
| REGN17516 | 2 | conformational | 289-297 | PIVNAELSF | 538 |
| (H1H12850B) | | | | | |
| REGN17516 | 3 | conformational | 564-572 | PYLGTTMDT | 539 |
| (H1H12850B) | | | | | |
| REGN17517 | 1 | linear | 243-246 | FEDL | 521 |
| (H1H31874B) | | | | | |

The extracellular unit of hTfR is structurally categorized into three domains, the helical, protease-like and apical domains (PDB 1SUV).

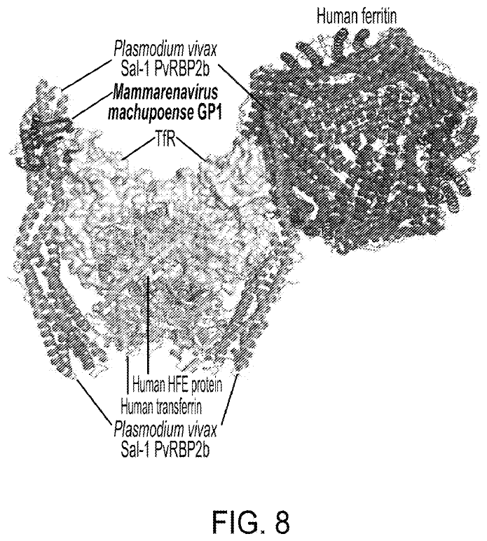

Structural studies of TfR in complex with a variety of molecules that have identified TfR binding sites, including Mammarenavirus machupoense GP1 protein (PDB 3KAS), canine parvovirus (PDB 2NSU), human ferritin (PDB 6GSR), Plasmodium vivax Sal-1 PvRBP2b (PDB 6D04), human HFE protein (PDB 1DE4), human transferrin (PDB 1SUV), etc. FIG. 8 shows the interactions of the above-listed molecules superimposed on a single TfR molecule.

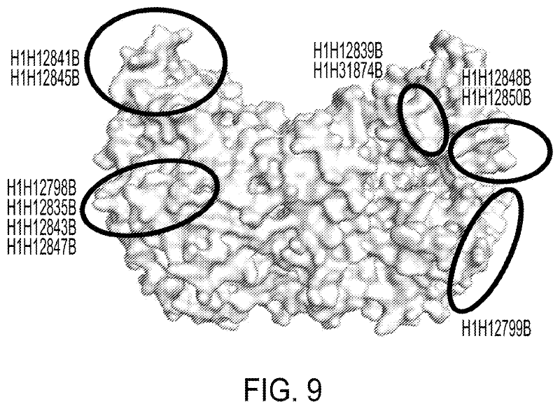

HDX protections for the antibodies tested in HDX-MS experiments can be assigned to 5 regions in TfR (PDB 1SUV) as depicted in FIG. 9.

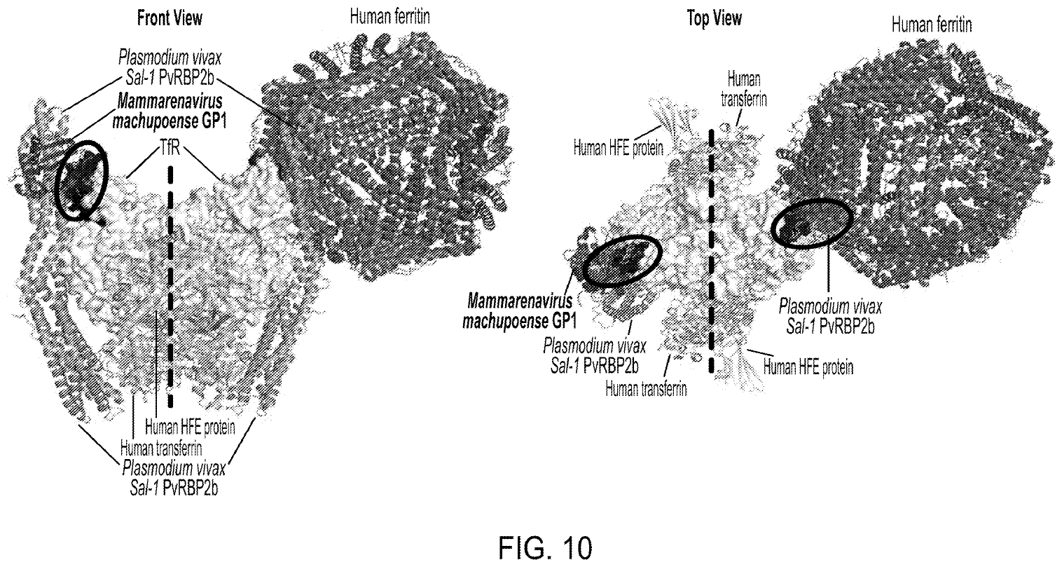

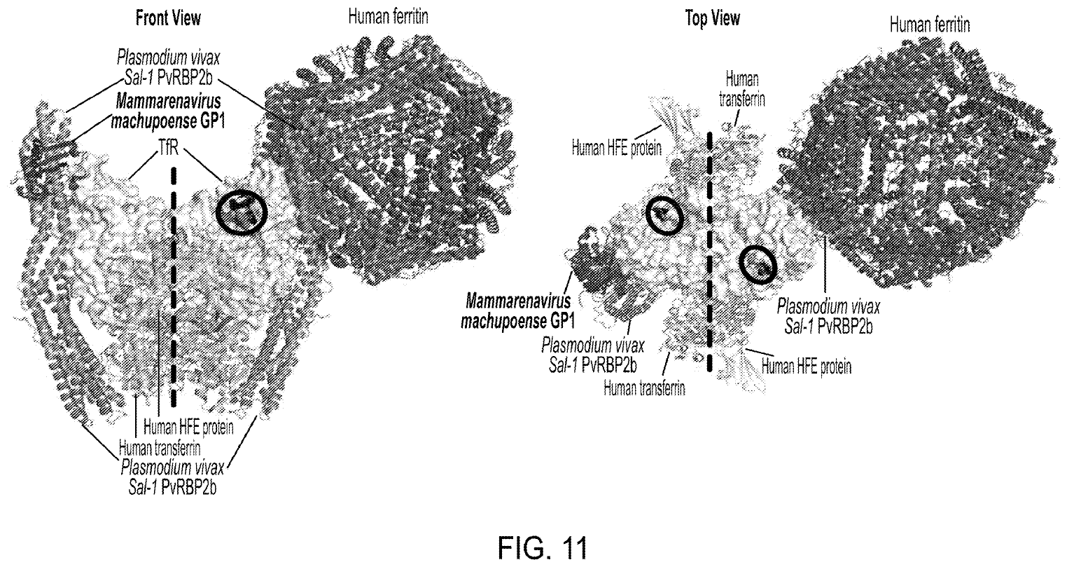

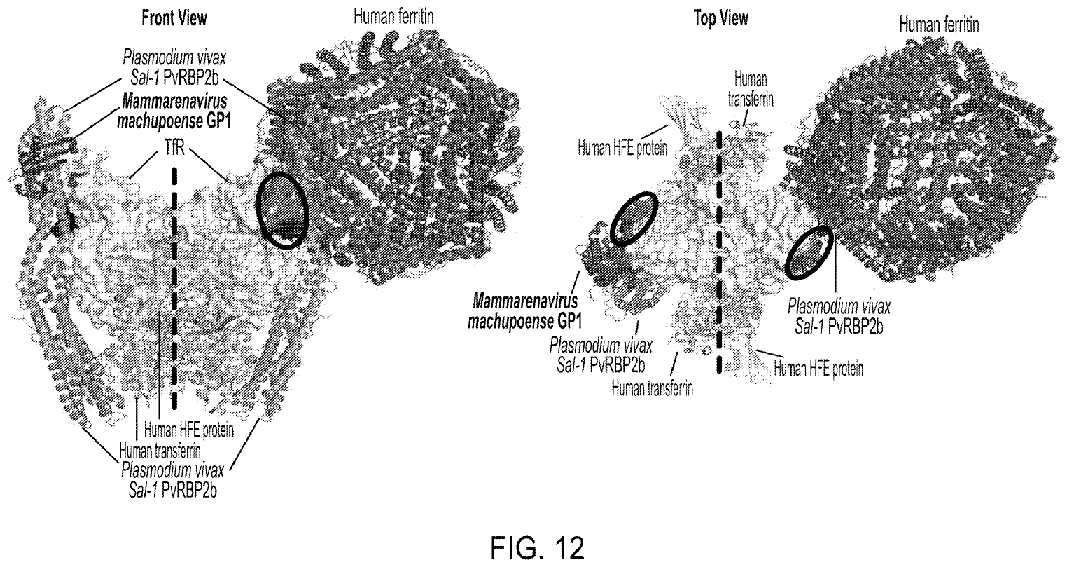

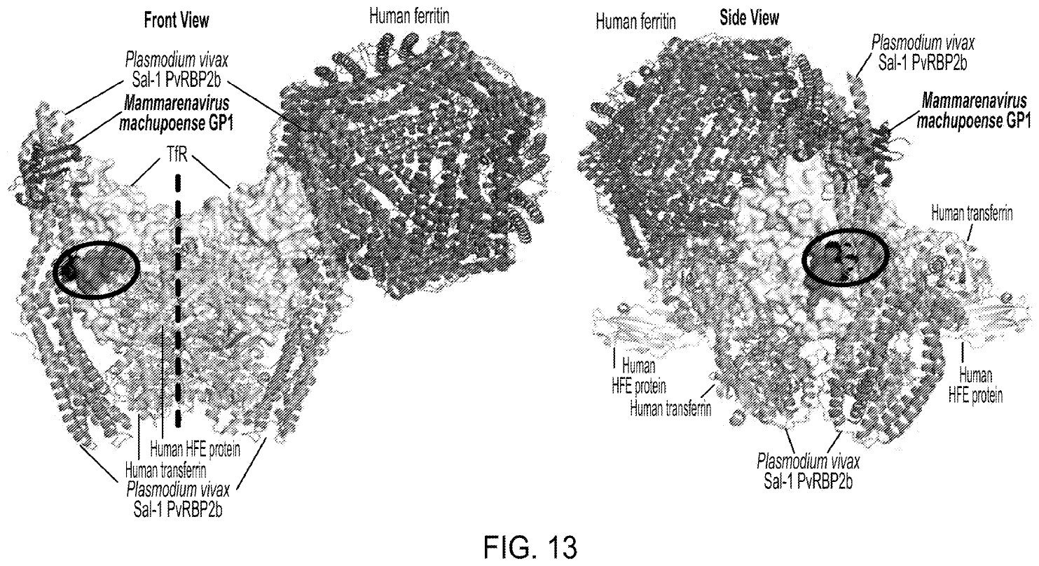

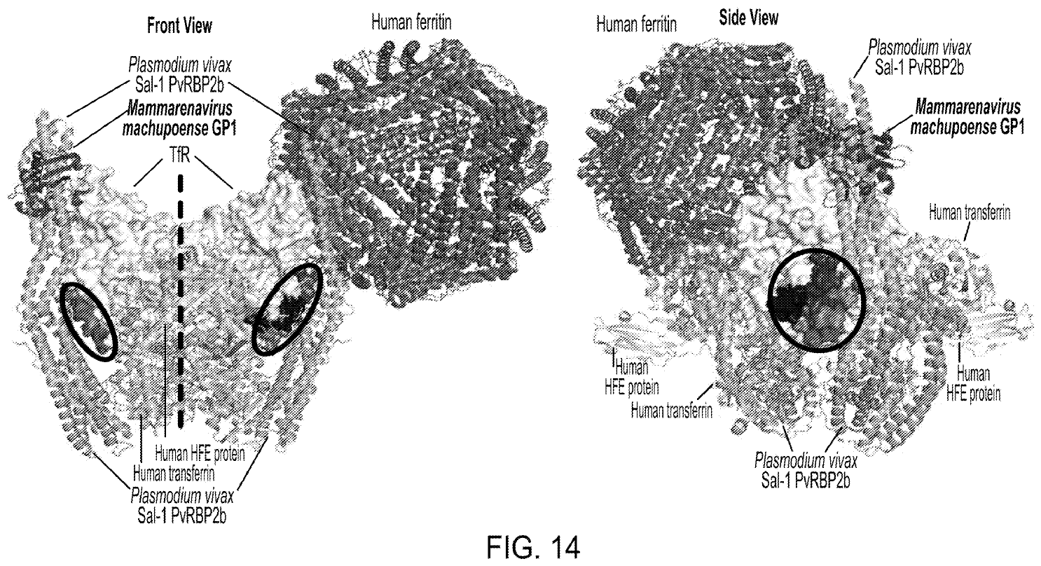

Tabulated summaries of data of the present Example are described in Tables 6-4 to Table 6-8. FIGS. 10-14 correspond to the tables below.

TABLE 6-4
Antibodies that show HDX protections in TfR apical domain and
overlap with Mammarenavirus machupoense GP1, canine parvovirus,
human ferritin, and plasmodium vivax Sal-1 PvRBP2b binding sites

| | | | m/h TfR residues with | |
|---|---|---|---|---|
| | | | significant changes in | Sequence |
| Antibody | REGN# | Antigen | deuterium % | coverage |
| H1H12841B | REGN17511 | hTfR.mmh | 199-224 | ~92.9% |
| | | | SVIIVDKNGRLVYLVENPGG | |
| | | | YVAYSK | |
| | | | (SEQ ID NO: 515) | |
| H1H12845B | REGN17513 | hmm.hTfR | 199-222 | ~88.1% |
| | | | SVIIVDKNGRLVYLVENPGG | |
| | | | YVAY | |
| | | | (SEQ ID NO: 519]) | |

TABLE 6-5
Antibodies with HDX protections in TfR apical domain that are not
shared by other TfR binding partners listed in Table 6-3

| | | | m/h TfR residues with | |
|---|---|---|---|---|
| | | | significant changes in | Sequence |
| Antibody | REGN# | Antigen | deuterium % | coverage |
| H1H31874B | REGN17517 | hTfR.mmh | 243-246 | ~92.9% |
| | | | FEDL | |
| | | | (SEQ ID NO: 521) | |
| H1H12839B | REGN17510 | hmm.hTfR | 238-246 | ~88.3% |
| | | | GTKKDFEDL | |
| | | | (SEQ ID NO: 514) | |

TABLE 6-6
Antibodies with HDX protections in TfR apical domain that share
binding sites with human ferritin and plasmodium vivax Sal-1 PvRBP2b

| | | | m/h TfR residues with | |
|---|---|---|---|---|
| | | | significant changes in | Sequence |
| Antibody | REGN# | Antigen | deuterium % | coverage |
| H1H12848B | REGN17515 | hTfR.mmh | 281-295 | ~92.9% |
| | | | IYMDQTKFPIVNAEL | |
| | | | (SEQ ID NO: 508) | |
| | | | 346-365 | |
| | | | FGNMEGDCPSDWKTDSTC | |
| | | | RM | |
| | | | (SEQ ID NO: 520) | |
| H1H12850B | REGN17516 | hTfR.mmh | 146-167 | ~92.9% |
| | | | LLNENSYVPREAGSQKDEN | |
| | | | LAL | |
| | | | (SEQ ID NO: 507) | |
| | | | 212-232 | |
| | | | LVENPGGYVAYSKAATVTG | |
| | | | KL | |
| | | | (SEQ ID NO: 522) | |
| | | | 281-297 | |
| | | | IYMDQTKFPIVNAELSF | |
| | | | (SEQ ID NO: 523) | |
| | | | 337-345 | |
| | | | ISRAAAEKL | |
| | | | (SEQ ID NO: 524) | |
| | | | 366-383 | |
| | | | VTSESKNVKLTVSNVLKE | |
| | | | (SEQ ID NO: 525) | |
| | | | 557-572 | |
| | | | FCEDTDYPYLGTTMDT | |
| | | | (SEQ ID NO: 526) | |

TABLE 6-7
Antibodies with HDX protections in TfR protease-like domain and
share binding sites with plasmodium vivax Sal-1 PvRBP2b

| | | | m/h TfR residues with | |
|---|---|---|---|---|
| | | | significant changes in | Sequence |
| Antibody | REGN# | Antigen | deuterium % | coverage |
| H1H12798B | REGN17507 | hmm.hTfR | 146-167 | ~87.0% |
| | | | LLNENSYVPREAGSQKDEN | |
| | | | LAL | |
| | | | (SEQ ID NO: 507) | |
| | | | 281-295 | |
| | | | IYMDQTKFPIVNAEL | |
| | | | (SEQ ID NO: 508) | |
| | | | 572-576 | |
| | | | TYKEL | |
| | | | (SEQ ID NO: 509) | |
| H1H12843B | REGN17512 | hmm.hTfR | 146-164 | ~88.3% |
| | | | LLNENSYVPREAGSQKDEN | |
| | | | (SEQ ID NO: 516) | |
| | | | 284-295 | |
| | | | DQTKFPIVNAEL | |
| | | | (SEQ ID NO: 517) | |
| | | | 572-585 | |
| | | | TYKELIERIPELNK | |
| | | | (SEQ ID NO: 518) | |
| H1H12847B | REGN17514 | hmm.hTfR | 146-164 | ~88.1% |
| | | | LLNENSYVPREAGSQKDEN | |
| | | | (SEQ ID NO: 516) | |
| | | | 572-585 | |
| | | | TYKELIERIPELNK | |
| | | | (SEQ ID NO: 518) | |
| H1H12835B | REGN17509 | hTfR.mmh | 147-165 | ~92.9% |
| | | | LNENSYVPREAGSQKDENL | |
| | | | (SEQ ID NO: 513) | |

TABLE 6-8
Antibodies with HDX protections in TfR protease-like domain. This region
is not utilized by other TfR interacting molecules listed in Table 6-6.

| | | | m/h TfR residues with | |
|---|---|---|---|---|
| | | | significant changes in | Sequence |
| Antibody | REGN# | Antigen | deuterium % | coverage |
| H1H12799B | REGN17508 | hmm.hTfR | 128-146 | ~88.7% |
| | | | KRKLSEKLDSTDFTGTIKL | |
| | | | (SEQ ID NO: 510) | |
| | | | 503-522 | |
| | | | YTLIEKTMQNVKHPVTGQFL | |
| | | | (SEQ ID NO: 511) | |
| | | | 576-592 | |
| | | | LIERIPELNKVARAAAE | |
| | | | (SEQ ID NO: 512) | |

### REFERENCES

1. Ehring (1999) Analytical Biochemistry 267(2):252-259
2. Engen and Smith (2001) Anal. Chem. 73:256A-265A

All references cited herein are incorporated by reference to the same extent as if each individual publication, database entry (e.g., Genbank sequences or GenelD entries), patent application, or patent, was specifically and individually indicated to be incorporated by reference. This statement of incorporation by reference is intended by Applicants to relate to each and every individual publication, database entry (e.g., Genbank sequences or GenelD entries), patent application, or patent, each of which is clearly identified in even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

We claim:

1. A protein-drug conjugate comprising an antigen-binding protein that binds specifically to human transferrin receptor, wherein the antigen-binding protein is conjugated to a molecular cargo, and wherein the antigen-binding protein binds to human transferrin receptor with a K_{D} of about 41 nM or a stronger affinity.

2. The protein-drug conjugate of claim 1 wherein the antigen binding protein comprises an antibody or antigen-binding fragment thereof.

3. The protein-drug conjugate of claim 2, wherein the antigen-binding protein is selected from a humanized antibody or antigen binding fragment thereof, human antibody or antigen binding fragment thereof, murine antibody or antigen binding fragment thereof, chimeric antibody or antigen binding fragment thereof, monovalent Fab′, divalent Fab2, F(ab)′3 fragments, single-chain fragment variable (scFv), bis-scFv, (scFv)2, diabody, bivalent antibody, one-armed antibody, minibody, nanobody, triabody, tetrabody, disulfide stabilized Fv protein (dsFv), single-domain antibody (sdAb), Ig NAR, single heavy chain antibody, bispecific antibody or binding fragment thereof, bi-specific T-cell engager (BiTE), trispecific antibody, or chemically modified derivatives thereof.

4. The protein-drug conjugate of any one of claim 1-3, wherein the antigen-binding protein comprises a fragment antigen-binding region (Fab).

5. The protein-drug conjugate of any one of claim 1-3, wherein the antigen-binding protein comprises a single chain fragment variable (scFv).

6. The protein-drug conjugate of claim 5, wherein the scFv comprises a heavy chain variable region (HCVR or V_{H}) and a light chain variable region (LCVR or V_{L}) arranged in the following orientation from N-terminus to C-terminus: HCVR-LCVR.

7. The protein-drug conjugate of claim 5, wherein the scFv comprises a HCVR and a LCVR arranged in the following orientation from N-terminus to C-terminus: LCVR-HCVR.

8. The protein-drug conjugate of any one of claims 5-7 wherein said scFv variable regions are connected by a linker.

9. The protein-drug conjugate of claim 8, wherein the linker is a peptide linker.

10. The protein-drug conjugate of claim 9 wherein the peptide linker is -(GGGGS)ₙ-(SEQ ID NO: 426); wherein n is 1, 2, 3, 4, 5, 6, 7, 8, 9 or 10.

11. The protein-drug conjugate of any one of claim 1-3, wherein the antigen-binding protein comprises a bivalent antibody.

12. The protein-drug conjugate of any one of claim 1-3, wherein the antigen-binding protein comprises a one-armed antibody.

13. The protein-drug conjugate of any one of claim 1-12 wherein the antigen-binding protein binds to human transferrin receptor with a K_{D} of about 3 nM or a stronger affinity.

14. The protein-drug conjugate of claim 13 wherein the antigen-binding protein binds to human transferrin receptor with a K_{D} of about 0.45 nM to 3 nM.

15. The protein-drug conjugate of any one of claim 1-13, wherein the antigen-binding protein comprises:
(i) a HCVR that comprises the HCDR1, HCDR2 and HCDR3 of a HCVR comprising the amino acid sequence set forth in SEQ ID NO: 2; 12; 22; 32; 42; 52; 62; 72; 82; 92; 102; 112; 122; 132; 142; 152; 162; 172; 182; 192; 202; 212; 222; 232; 242; 252; 262; 272; 282; 292; 302; or 312 (or a variant thereof);
and/or
(ii) a LCVR that comprises the LCDR1, LCDR2 and LCDR3 of a LCVR comprising the amino acid sequence set forth in SEQ ID NO: 7; 17; 27; 37; 47; 57; 67; 77; 87; 97; 107; 117; 127; 137; 147; 157; 167; 177; 187; 197; 207; 217; 227; 237; 247; 257; 267; 277; 287; 297; 307; or 317 (or a variant thereof).

16. The protein-drug conjugate of any one of claim 1-15, wherein the antigen-binding protein comprises:
(1) a HCVR comprising the HCDR1, HCDR2 and HCDR3 of a HCVR that comprises the amino acid sequence set forth in SEQ ID NO: 2 (or a variant thereof); and a LCVR comprising the LCDR1, LCDR2 and LCDR3 of a LCVR that comprises the amino acid sequence set forth in SEQ ID NO: 7 (or a variant thereof);
(2) a HCVR comprising the HCDR1, HCDR2 and HCDR3 of a HCVR that comprises the amino acid sequence set forth in SEQ ID NO: 12 (or a variant thereof); and a LCVR comprising the LCDR1, LCDR2 and LCDR3 of a LCVR that comprises the amino acid sequence set forth in SEQ ID NO: 17 (or a variant thereof);
(3) a HCVR comprising the HCDR1, HCDR2 and HCDR3 of a HCVR that comprises the amino acid sequence set forth in SEQ ID NO: 22 (or a variant thereof); and a LCVR comprising the LCDR1, LCDR2 and LCDR3 of a LCVR that comprises the amino acid sequence set forth in SEQ ID NO: 27 (or a variant thereof);
(4) a HCVR comprising the HCDR1, HCDR2 and HCDR3 of a HCVR that comprises the amino acid sequence set forth in SEQ ID NO: 32 (or a variant thereof); and a LCVR comprising the LCDR1, LCDR2 and LCDR3 of a LCVR that comprises the amino acid sequence set forth in SEQ ID NO: 37 or 465 (or a variant thereof);
(5) a HCVR comprising the HCDR1, HCDR2 and HCDR3 of a HCVR that comprises the amino acid sequence set forth in SEQ ID NO: 42 (or a variant thereof); and a LCVR comprising the LCDR1, LCDR2 and LCDR3 of a LCVR that comprises the amino acid sequence set forth in SEQ ID NO: 47 (or a variant thereof);
(6) a HCVR comprising the HCDR1, HCDR2 and HCDR3 of a HCVR that comprises the amino acid sequence set forth in SEQ ID NO: 52 (or a variant thereof); and a LCVR comprising the LCDR1, LCDR2 and LCDR3 of a LCVR that comprises the amino acid sequence set forth in SEQ ID NO: 57 (or a variant thereof);
(7) a HCVR comprising the HCDR1, HCDR2 and HCDR3 of a HCVR that comprises the amino acid sequence set forth in SEQ ID NO: 62 (or a variant thereof); and a LCVR comprising the LCDR1, LCDR2 and LCDR3 of a LCVR that comprises the amino acid sequence set forth in SEQ ID NO: 67 (or a variant thereof);
(8) a HCVR comprising the HCDR1, HCDR2 and HCDR3 of a HCVR that comprises the amino acid sequence set forth in SEQ ID NO: 72 (or a variant thereof); and a LCVR comprising the LCDR1, LCDR2 and LCDR3 of a LCVR that comprises the amino acid sequence set forth in SEQ ID NO: 77 (or a variant thereof);
(9) a HCVR comprising the HCDR1, HCDR2 and HCDR3 of a HCVR that comprises the amino acid sequence set forth in SEQ ID NO: 82 (or a variant thereof); and a LCVR comprising the LCDR1, LCDR2 and LCDR3 of a LCVR that comprises the amino acid sequence set forth in SEQ ID NO: 87 (or a variant thereof);
(10) a HCVR comprising the HCDR1, HCDR2 and HCDR3 of a HCVR that comprises the amino acid sequence set forth in SEQ ID NO: 92 (or a variant thereof); and a LCVR comprising the LCDR1, LCDR2 and LCDR3 of a LCVR that comprises the amino acid sequence set forth in SEQ ID NO: 97 (or a variant thereof);
(11) a HCVR comprising the HCDR1, HCDR2 and HCDR3 of a HCVR that comprises the amino acid sequence set forth in SEQ ID NO: 102 (or a variant thereof); and a LCVR comprising the LCDR1, LCDR2 and LCDR3 of a LCVR that comprises the amino acid sequence set forth in SEQ ID NO: 107 (or a variant thereof);
(12) a HCVR comprising the HCDR1, HCDR2 and HCDR3 of a HCVR that comprises the amino acid sequence set forth in SEQ ID NO: 112 (or a variant thereof); and a LCVR comprising the LCDR1, LCDR2 and LCDR3 of a LCVR that comprises the amino acid sequence set forth in SEQ ID NO: 117 (or a variant thereof);
(13) a HCVR comprising the HCDR1, HCDR2 and HCDR3 of a HCVR that comprises the amino acid sequence set forth in SEQ ID NO: 122 (or a variant thereof); and a LCVR comprising the LCDR1, LCDR2 and LCDR3 of a LCVR that comprises the amino acid sequence set forth in SEQ ID NO: 127 (or a variant thereof);
(14) a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof);
(15) a HCVR comprising the HCDR1, HCDR2 and HCDR3 of a HCVR that comprises the amino acid sequence set forth in SEQ ID NO: 142 (or a variant thereof); and a LCVR comprising the LCDR1, LCDR2 and LCDR3 of a LCVR that comprises the amino acid sequence set forth in SEQ ID NO: 147 (or a variant thereof);
(16) a HCVR comprising the HCDR1, HCDR2 and HCDR3 of a HCVR that comprises the amino acid sequence set forth in SEQ ID NO: 152 (or a variant thereof); and a LCVR comprising the LCDR1, LCDR2 and LCDR3 of a LCVR that comprises the amino acid sequence set forth in SEQ ID NO: 157 (or a variant thereof);
(17) a HCVR comprising the HCDR1, HCDR2 and HCDR3 of a HCVR that comprises the amino acid sequence set forth in SEQ ID NO: 162 (or a variant thereof); and a LCVR comprising the LCDR1, LCDR2 and LCDR3 of a LCVR that comprises the amino acid sequence set forth in SEQ ID NO: 167 (or a variant thereof);
(18) a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof);
(19) a HCVR comprising the HCDR1, HCDR2 and HCDR3 of a HCVR that comprises the amino acid sequence set forth in SEQ ID NO: 182 (or a variant thereof); and a LCVR comprising the LCDR1, LCDR2 and LCDR3 of a LCVR that comprises the amino acid sequence set forth in SEQ ID NO: 187 (or a variant thereof);
(20) a HCVR comprising the HCDR1, HCDR2 and HCDR3 of a HCVR that comprises the amino acid sequence set forth in SEQ ID NO: 192 (or a variant thereof); and a LCVR comprising the LCDR1, LCDR2 and LCDR3 of a LCVR that comprises the amino acid sequence set forth in SEQ ID NO: 197 (or a variant thereof);
(21) a HCVR comprising the HCDR1, HCDR2 and HCDR3 of a HCVR that comprises the amino acid sequence set forth in SEQ ID NO: 202 (or a variant thereof); and a LCVR comprising the LCDR1, LCDR2 and LCDR3 of a LCVR that comprises the amino acid sequence set forth in SEQ ID NO: 207 (or a variant thereof);
(22) a HCVR comprising the HCDR1, HCDR2 and HCDR3 of a HCVR that comprises the amino acid sequence set forth in SEQ ID NO: 212 (or a variant thereof); and a LCVR comprising the LCDR1, LCDR2 and LCDR3 of a LCVR that comprises the amino acid sequence set forth in SEQ ID NO: 217 (or a variant thereof);
(23) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof);
(24) a HCVR comprising the HCDR1, HCDR2 and HCDR3 of a HCVR that comprises the amino acid sequence set forth in SEQ ID NO: 232 (or a variant thereof); and a LCVR comprising the LCDR1, LCDR2 and LCDR3 of a LCVR that comprises the amino acid sequence set forth in SEQ ID NO: 237 (or a variant thereof);
(25) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof);
(26) a HCVR comprising the HCDR1, HCDR2 and HCDR3 of a HCVR that comprises the amino acid sequence set forth in SEQ ID NO: 252 (or a variant thereof); and a LCVR comprising the LCDR1, LCDR2 and LCDR3 of a LCVR that comprises the amino acid sequence set forth in SEQ ID NO: 257 (or a variant thereof);
(27) a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof);
(28) a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof);
(29) a HCVR comprising the HCDR1, HCDR2 and HCDR3 of a HCVR that comprises the amino acid sequence set forth in SEQ ID NO: 282 (or a variant thereof); and a LCVR comprising the LCDR1, LCDR2 and LCDR3 of a LCVR that comprises the amino acid sequence set forth in SEQ ID NO: 287 (or a variant thereof);
(30) a HCVR comprising the HCDR1, HCDR2 and HCDR3 of a HCVR that comprises the amino acid sequence set forth in SEQ ID NO: 292 (or a variant thereof); and a LCVR comprising the LCDR1, LCDR2 and LCDR3 of a LCVR that comprises the amino acid sequence set forth in SEQ ID NO: 297 (or a variant thereof);
(31) a HCVR comprising the HCDR1, HCDR2 and HCDR3 of a HCVR that comprises the amino acid sequence set forth in SEQ ID NO: 302 (or a variant thereof); and a LCVR comprising the LCDR1, LCDR2 and LCDR3 of a LCVR that comprises the amino acid sequence set forth in SEQ ID NO: 307 (or a variant thereof); and/or
(32) a HCVR comprising the HCDR1, HCDR2 and HCDR3 of a HCVR that comprises the amino acid sequence set forth in SEQ ID NO: 312 (or a variant thereof); and a LCVR comprising the LCDR1, LCDR2 and LCDR3 of a LCVR that comprises the amino acid sequence set forth in SEQ ID NO: 317 (or a variant thereof).

17. The protein-drug conjugate of any one of claim 1-16, wherein the antigen-binding protein comprises:
(a) a HCVR that comprises:
an HCDR1 comprising the amino acid sequence set forth in SEQ ID NO: 3 (or a variant thereof),
an HCDR2 comprising the amino acid sequence set forth in SEQ ID NO: 4 (or a variant thereof), and
an HCDR3 comprising the amino acid sequence set forth in SEQ ID NO: 5 (or a variant thereof); and
a LCVR that comprises:
an LCDR1 comprising the amino acid sequence set forth in SEQ ID NO: 8 (or a variant thereof),
an LCDR2 comprising the amino acid sequence set forth in SEQ ID NO: 9 (or a variant thereof), and
an LCDR3 comprising the amino acid sequence set forth in SEQ ID NO: 10 (or a variant thereof);
(b) a HCVR that comprises:
an HCDR1 comprising the amino acid sequence set forth in SEQ ID NO: 13 (or a variant thereof),
an HCDR2 comprising the amino acid sequence set forth in SEQ ID NO: 14 (or a variant thereof), and
an HCDR3 comprising the amino acid sequence set forth in SEQ ID NO: 15 (or a variant thereof); and
a LCVR that comprises:
an LCDR1 comprising the amino acid sequence set forth in SEQ ID NO: 18 (or a variant thereof),
an LCDR2 comprising the amino acid sequence set forth in SEQ ID NO: 19 (or a variant thereof), and
an LCDR3 comprising the amino acid sequence set forth in SEQ ID NO: 20 (or a variant thereof);
(c) a HCVR that comprises:
an HCDR1 comprising the amino acid sequence set forth in SEQ ID NO: 23 (or a variant thereof),
an HCDR2 comprising the amino acid sequence set forth in SEQ ID NO: 24 (or a variant thereof), and
an HCDR3 comprising the amino acid sequence set forth in SEQ ID NO: 25 (or a variant thereof); and
a LCVR that comprises:
an LCDR1 comprising the amino acid sequence set forth in SEQ ID NO: 28 (or a variant thereof),
an LCDR2 comprising the amino acid sequence set forth in SEQ ID NO: 29 (or a variant thereof), and
an LCDR3 comprising the amino acid sequence set forth in SEQ ID NO: 30 (or a variant thereof);
(d) a HCVR that comprises:
an HCDR1 comprising the amino acid sequence set forth in SEQ ID NO: 33 (or a variant thereof),
an HCDR2 comprising the amino acid sequence set forth in SEQ ID NO: 34 (or a variant thereof), and
an HCDR3 comprising the amino acid sequence set forth in SEQ ID NO: 35 (or a variant thereof); and
a LCVR that comprises:
an LCDR1 comprising the amino acid sequence set forth in SEQ ID NO: 38 (or a variant thereof),
an LCDR2 comprising the amino acid sequence set forth in SEQ ID NO: 39 (or a variant thereof), and
an LCDR3 comprising the amino acid sequence set forth in SEQ ID NO: 40 (or a variant thereof);
(e) a HCVR that comprises:
an HCDR1 comprising the amino acid sequence set forth in SEQ ID NO: 43 (or a variant thereof),
an HCDR2 comprising the amino acid sequence set forth in SEQ ID NO: 44 (or a variant thereof), and
an HCDR3 comprising the amino acid sequence set forth in SEQ ID NO: 45 (or a variant thereof); and
a LCVR that comprises:
an LCDR1 comprising the amino acid sequence set forth in SEQ ID NO: 48 (or a variant thereof),
an LCDR2 comprising the amino acid sequence set forth in SEQ ID NO: 49 (or a variant thereof), and
an LCDR3 comprising the amino acid sequence set forth in SEQ ID NO: 50 (or a variant thereof);
(f) a HCVR that comprises:
an HCDR1 comprising the amino acid sequence set forth in SEQ ID NO: 53 (or a variant thereof),
an HCDR2 comprising the amino acid sequence set forth in SEQ ID NO: 54 (or a variant thereof), and
an HCDR3 comprising the amino acid sequence set forth in SEQ ID NO: 55 (or a variant thereof); and
a LCVR that comprises:
an LCDR1 comprising the amino acid sequence set forth in SEQ ID NO: 58 (or a variant thereof),
an LCDR2 comprising the amino acid sequence set forth in SEQ ID NO: 59 (or a variant thereof), and
an LCDR3 comprising the amino acid sequence set forth in SEQ ID NO: 60 (or a variant thereof);
(g) a HCVR that comprises:
an HCDR1 comprising the amino acid sequence set forth in SEQ ID NO: 63 (or a variant thereof),
an HCDR2 comprising the amino acid sequence set forth in SEQ ID NO: 64 (or a variant thereof), and
an HCDR3 comprising the amino acid sequence set forth in SEQ ID NO: 65 (or a variant thereof); and
a LCVR that comprises:
an LCDR1 comprising the amino acid sequence set forth in SEQ ID NO: 68 (or a variant thereof),
an LCDR2 comprising the amino acid sequence set forth in SEQ ID NO: 69 (or a variant thereof), and
an LCDR3 comprising the amino acid sequence set forth in SEQ ID NO: 70 (or a variant thereof);
(h) a HCVR that comprises:
an HCDR1 comprising the amino acid sequence set forth in SEQ ID NO: 73 (or a variant thereof),
an HCDR2 comprising the amino acid sequence set forth in SEQ ID NO: 74 (or a variant thereof), and
an HCDR3 comprising the amino acid sequence set forth in SEQ ID NO: 75 (or a variant thereof); and
a LCVR that comprises:
an LCDR1 comprising the amino acid sequence set forth in SEQ ID NO: 78 (or a variant thereof),
an LCDR2 comprising the amino acid sequence set forth in SEQ ID NO: 79 (or a variant thereof), and
an LCDR3 comprising the amino acid sequence set forth in SEQ ID NO: 80 (or a variant thereof);
(i) a HCVR that comprises:
an HCDR1 comprising the amino acid sequence set forth in SEQ ID NO: 83 (or a variant thereof),
an HCDR2 comprising the amino acid sequence set forth in SEQ ID NO: 84 (or a variant thereof), and
an HCDR3 comprising the amino acid sequence set forth in SEQ ID NO: 85 (or a variant thereof); and
a LCVR that comprises:
an LCDR1 comprising the amino acid sequence set forth in SEQ ID NO: 88 (or a variant thereof),
an LCDR2 comprising the amino acid sequence set forth in SEQ ID NO: 89 (or a variant thereof), and
an LCDR3 comprising the amino acid sequence set forth in SEQ ID NO: 90 (or a variant thereof);
(j) a HCVR that comprises:
an HCDR1 comprising the amino acid sequence set forth in SEQ ID NO: 93 (or a variant thereof),
an HCDR2 comprising the amino acid sequence set forth in SEQ ID NO: 94 (or a variant thereof), and
an HCDR3 comprising the amino acid sequence set forth in SEQ ID NO: 95 (or a variant thereof); and
a LCVR that comprises:
an LCDR1 comprising the amino acid sequence set forth in SEQ ID NO: 98 (or a variant thereof),
an LCDR2 comprising the amino acid sequence set forth in SEQ ID NO: 99 (or a variant thereof), and
an LCDR3 comprising the amino acid sequence set forth in SEQ ID NO: 100 (or a variant thereof);
(k) a HCVR that comprises:
an HCDR1 comprising the amino acid sequence set forth in SEQ ID NO: 103 (or a variant thereof),
an HCDR2 comprising the amino acid sequence set forth in SEQ ID NO: 104 (or a variant thereof), and
an HCDR3 comprising the amino acid sequence set forth in SEQ ID NO: 105 (or a variant thereof); and
a LCVR that comprises:
an LCDR1 comprising the amino acid sequence set forth in SEQ ID NO: 108 (or a variant thereof),
an LCDR2 comprising the amino acid sequence set forth in SEQ ID NO: 109 (or a variant thereof), and
an LCDR3 comprising the amino acid sequence set forth in SEQ ID NO: 110 (or a variant thereof);
(l) a HCVR that comprises:
an HCDR1 comprising the amino acid sequence set forth in SEQ ID NO: 113 (or a variant thereof),
an HCDR2 comprising the amino acid sequence set forth in SEQ ID NO: 114 (or a variant thereof), and
an HCDR3 comprising the amino acid sequence set forth in SEQ ID NO: 115 (or a variant thereof); and
a LCVR that comprises:
an LCDR1 comprising the amino acid sequence set forth in SEQ ID NO: 118 (or a variant thereof),
an LCDR2 comprising the amino acid sequence set forth in SEQ ID NO: 119 (or a variant thereof), and
an LCDR3 comprising the amino acid sequence set forth in SEQ ID NO: 120 (or a variant thereof);
(m) a HCVR that comprises:
an HCDR1 comprising the amino acid sequence set forth in SEQ ID NO: 123 (or a variant thereof),
an HCDR2 comprising the amino acid sequence set forth in SEQ ID NO: 124 (or a variant thereof), and
an HCDR3 comprising the amino acid sequence set forth in SEQ ID NO: 125 (or a variant thereof); and
a LCVR that comprises:
an LCDR1 comprising the amino acid sequence set forth in SEQ ID NO: 128 (or a variant thereof),
an LCDR2 comprising the amino acid sequence set forth in SEQ ID NO: 129 (or a variant thereof), and
an LCDR3 comprising the amino acid sequence set forth in SEQ ID NO: 130 (or a variant thereof);
(n) a HCVR that comprises:
an HCDR1 comprising the amino acid sequence set forth in SEQ ID NO: 133 (or a variant thereof),
an HCDR2 comprising the amino acid sequence set forth in SEQ ID NO: 134 (or a variant thereof), and
an HCDR3 comprising the amino acid sequence set forth in SEQ ID NO: 135 (or a variant thereof); and
a LCVR that comprises:
an LCDR1 comprising the amino acid sequence set forth in SEQ ID NO: 138 (or a variant thereof),
an LCDR2 comprising the amino acid sequence set forth in SEQ ID NO: 139 (or a variant thereof), and
an LCDR3 comprising the amino acid sequence set forth in SEQ ID NO: 140 (or a variant thereof);
(o) a HCVR that comprises:
an HCDR1 comprising the amino acid sequence set forth in SEQ ID NO: 143 (or a variant thereof),
an HCDR2 comprising the amino acid sequence set forth in SEQ ID NO: 144 (or a variant thereof), and
an HCDR3 comprising the amino acid sequence set forth in SEQ ID NO: 145 (or a variant thereof); and
a LCVR that comprises:
an LCDR1 comprising the amino acid sequence set forth in SEQ ID NO: 148 (or a variant thereof),
an LCDR2 comprising the amino acid sequence set forth in SEQ ID NO: 149 (or a variant thereof), and
an LCDR3 comprising the amino acid sequence set forth in SEQ ID NO: 150 (or a variant thereof);
(p) a HCVR that comprises:
an HCDR1 comprising the amino acid sequence set forth in SEQ ID NO: 153 (or a variant thereof),
an HCDR2 comprising the amino acid sequence set forth in SEQ ID NO: 154 (or a variant thereof), and
an HCDR3 comprising the amino acid sequence set forth in SEQ ID NO: 155 (or a variant thereof); and
a LCVR that comprises:
an LCDR1 comprising the amino acid sequence set forth in SEQ ID NO: 158 (or a variant thereof),
an LCDR2 comprising the amino acid sequence set forth in SEQ ID NO: 159 (or a variant thereof), and
an LCDR3 comprising the amino acid sequence set forth in SEQ ID NO: 160 (or a variant thereof);
(q) a HCVR that comprises:
an HCDR1 comprising the amino acid sequence set forth in SEQ ID NO: 163 (or a variant thereof),
an HCDR2 comprising the amino acid sequence set forth in SEQ ID NO: 164 (or a variant thereof), and
an HCDR3 comprising the amino acid sequence set forth in SEQ ID NO: 165 (or a variant thereof); and
a LCVR that comprises:
an LCDR1 comprising the amino acid sequence set forth in SEQ ID NO: 168 (or a variant thereof),
an LCDR2 comprising the amino acid sequence set forth in SEQ ID NO: 169 (or a variant thereof), and
an LCDR3 comprising the amino acid sequence set forth in SEQ ID NO: 170 (or a variant thereof);
(r) a HCVR that comprises:
an HCDR1 comprising the amino acid sequence set forth in SEQ ID NO: 173 (or a variant thereof),
an HCDR2 comprising the amino acid sequence set forth in SEQ ID NO: 174 (or a variant thereof), and
an HCDR3 comprising the amino acid sequence set forth in SEQ ID NO: 175 (or a variant thereof); and
a LCVR that comprises:
an LCDR1 comprising the amino acid sequence set forth in SEQ ID NO: 178 (or a variant thereof),
an LCDR2 comprising the amino acid sequence set forth in SEQ ID NO: 179 (or a variant thereof), and
an LCDR3 comprising the amino acid sequence set forth in SEQ ID NO: 180 (or a variant thereof);
(s) a HCVR that comprises:
an HCDR1 comprising the amino acid sequence set forth in SEQ ID NO: 183 (or a variant thereof),
an HCDR2 comprising the amino acid sequence set forth in SEQ ID NO: 184 (or a variant thereof), and
an HCDR3 comprising the amino acid sequence set forth in SEQ ID NO: 185 (or a variant thereof); and
a LCVR that comprises:
an LCDR1 comprising the amino acid sequence set forth in SEQ ID NO: 188 (or a variant thereof),
an LCDR2 comprising the amino acid sequence set forth in SEQ ID NO: 189 (or a variant thereof), and
an LCDR3 comprising the amino acid sequence set forth in SEQ ID NO: 190 (or a variant thereof);
(t) a HCVR that comprises:
an HCDR1 comprising the amino acid sequence set forth in SEQ ID NO: 193 (or a variant thereof),
an HCDR2 comprising the amino acid sequence set forth in SEQ ID NO: 194 (or a variant thereof), and
an HCDR3 comprising the amino acid sequence set forth in SEQ ID NO: 195 (or a variant thereof); and
a LCVR that comprises:
an LCDR1 comprising the amino acid sequence set forth in SEQ ID NO: 198 (or a variant thereof),
an LCDR2 comprising the amino acid sequence set forth in SEQ ID NO: 199 (or a variant thereof), and
an LCDR3 comprising the amino acid sequence set forth in SEQ ID NO: 200 (or a variant thereof);
(u) a HCVR that comprises:
an HCDR1 comprising the amino acid sequence set forth in SEQ ID NO: 203 (or a variant thereof),
an HCDR2 comprising the amino acid sequence set forth in SEQ ID NO: 204 (or a variant thereof), and
an HCDR3 comprising the amino acid sequence set forth in SEQ ID NO: 205 (or a variant thereof); and
a LCVR that comprises:
an LCDR1 comprising the amino acid sequence set forth in SEQ ID NO: 208 (or a variant thereof),
an LCDR2 comprising the amino acid sequence set forth in SEQ ID NO: 209 (or a variant thereof), and
an LCDR3 comprising the amino acid sequence set forth in SEQ ID NO: 210 (or a variant thereof);
(v) a HCVR that comprises:
an HCDR1 comprising the amino acid sequence set forth in SEQ ID NO: 213 (or a variant thereof),
an HCDR2 comprising the amino acid sequence set forth in SEQ ID NO: 214 (or a variant thereof), and
an HCDR3 comprising the amino acid sequence set forth in SEQ ID NO: 215 (or a variant thereof); and
a LCVR that comprises:
an LCDR1 comprising the amino acid sequence set forth in SEQ ID NO: 218 (or a variant thereof),
an LCDR2 comprising the amino acid sequence set forth in SEQ ID NO: 219 (or a variant thereof), and
an LCDR3 comprising the amino acid sequence set forth in SEQ ID NO: 220 (or a variant thereof);
(w) a HCVR that comprises:
an HCDR1 comprising the amino acid sequence set forth in SEQ ID NO: 223 (or a variant thereof),
an HCDR2 comprising the amino acid sequence set forth in SEQ ID NO: 224 (or a variant thereof), and
an HCDR3 comprising the amino acid sequence set forth in SEQ ID NO: 225 (or a variant thereof); and
a LCVR that comprises:
an LCDR1 comprising the amino acid sequence set forth in SEQ ID NO: 228 (or a variant thereof),
an LCDR2 comprising the amino acid sequence set forth in SEQ ID NO: 229 (or a variant thereof), and
an LCDR3 comprising the amino acid sequence set forth in SEQ ID NO: 230 (or a variant thereof);
(x) a HCVR that comprises:
an HCDR1 comprising the amino acid sequence set forth in SEQ ID NO: 233 (or a variant thereof),
an HCDR2 comprising the amino acid sequence set forth in SEQ ID NO: 234 (or a variant thereof), and
an HCDR3 comprising the amino acid sequence set forth in SEQ ID NO: 235 (or a variant thereof); and
a LCVR that comprises:
an LCDR1 comprising the amino acid sequence set forth in SEQ ID NO: 238 (or a variant thereof),
an LCDR2 comprising the amino acid sequence set forth in SEQ ID NO: 239 (or a variant thereof), and
an LCDR3 comprising the amino acid sequence set forth in SEQ ID NO: 240 (or a variant thereof);
(y) a HCVR that comprises:
an HCDR1 comprising the amino acid sequence set forth in SEQ ID NO: 243 (or a variant thereof),
an HCDR2 comprising the amino acid sequence set forth in SEQ ID NO: 244 (or a variant thereof), and
an HCDR3 comprising the amino acid sequence set forth in SEQ ID NO: 245 (or a variant thereof); and
a LCVR that comprises:
an LCDR1 comprising the amino acid sequence set forth in SEQ ID NO: 248 (or a variant thereof),
an LCDR2 comprising the amino acid sequence set forth in SEQ ID NO: 249 (or a variant thereof), and
an LCDR3 comprising the amino acid sequence set forth in SEQ ID NO: 250 (or a variant thereof);
(z) a HCVR that comprises:
an HCDR1 comprising the amino acid sequence set forth in SEQ ID NO: 253 (or a variant thereof),
an HCDR2 comprising the amino acid sequence set forth in SEQ ID NO: 254 (or a variant thereof), and
an HCDR3 comprising the amino acid sequence set forth in SEQ ID NO: 255 (or a variant thereof); and
a LCVR that comprises:
an LCDR1 comprising the amino acid sequence set forth in SEQ ID NO: 258 (or a variant thereof),
an LCDR2 comprising the amino acid sequence set forth in SEQ ID NO: 259 (or a variant thereof), and
an LCDR3 comprising the amino acid sequence set forth in SEQ ID NO: 260 (or a variant thereof);
(aa) a HCVR that comprises:
an HCDR1 comprising the amino acid sequence set forth in SEQ ID NO: 263 (or a variant thereof),
an HCDR2 comprising the amino acid sequence set forth in SEQ ID NO: 264 (or a variant thereof), and
an HCDR3 comprising the amino acid sequence set forth in SEQ ID NO: 265 (or a variant thereof); and
a LCVR that comprises:
an LCDR1 comprising the amino acid sequence set forth in SEQ ID NO: 268 (or a variant thereof),
an LCDR2 comprising the amino acid sequence set forth in SEQ ID NO: 269 (or a variant thereof), and
an LCDR3 comprising the amino acid sequence set forth in SEQ ID NO: 270 (or a variant thereof);
(ab) a HCVR that comprises:
an HCDR1 comprising the amino acid sequence set forth in SEQ ID NO: 273 (or a variant thereof),
an HCDR2 comprising the amino acid sequence set forth in SEQ ID NO: 274 (or a variant thereof), and
an HCDR3 comprising the amino acid sequence set forth in SEQ ID NO: 275 (or a variant thereof); and
a LCVR that comprises:
an LCDR1 comprising the amino acid sequence set forth in SEQ ID NO: 278 (or a variant thereof),
an LCDR2 comprising the amino acid sequence set forth in SEQ ID NO: 279 (or a variant thereof), and
an LCDR3 comprising the amino acid sequence set forth in SEQ ID NO: 280 (or a variant thereof);
(ac) a HCVR that comprises:
an HCDR1 comprising the amino acid sequence set forth in SEQ ID NO: 283 (or a variant thereof),
an HCDR2 comprising the amino acid sequence set forth in SEQ ID NO: 284 (or a variant thereof), and
an HCDR3 comprising the amino acid sequence set forth in SEQ ID NO: 285 (or a variant thereof); and
a LCVR that comprises:
an LCDR1 comprising the amino acid sequence set forth in SEQ ID NO: 288 (or a variant thereof),
an LCDR2 comprising the amino acid sequence set forth in SEQ ID NO: 289 (or a variant thereof), and
an LCDR3 comprising the amino acid sequence set forth in SEQ ID NO: 290 (or a variant thereof);
(ad) a HCVR that comprises:
an HCDR1 comprising the amino acid sequence set forth in SEQ ID NO: 293 (or a variant thereof),
an HCDR2 comprising the amino acid sequence set forth in SEQ ID NO: 294 (or a variant thereof), and
an HCDR3 comprising the amino acid sequence set forth in SEQ ID NO: 295 (or a variant thereof); and
a LCVR that comprises:
an LCDR1 comprising the amino acid sequence set forth in SEQ ID NO: 298 (or a variant thereof),
an LCDR2 comprising the amino acid sequence set forth in SEQ ID NO: 299 (or a variant thereof), and
an LCDR3 comprising the amino acid sequence set forth in SEQ ID NO: 300 (or a variant thereof);
(ae) a HCVR that comprises:
an HCDR1 comprising the amino acid sequence set forth in SEQ ID NO: 303 (or a variant thereof),
an HCDR2 comprising the amino acid sequence set forth in SEQ ID NO: 304 (or a variant thereof), and
an HCDR3 comprising the amino acid sequence set forth in SEQ ID NO: 305 (or a variant thereof); and
a LCVR that comprises:
an LCDR1 comprising the amino acid sequence set forth in SEQ ID NO: 308 (or a variant thereof),
an LCDR2 comprising the amino acid sequence set forth in SEQ ID NO: 309 (or a variant thereof), and
an LCDR3 comprising the amino acid sequence set forth in SEQ ID NO: 310 (or a variant thereof);
and/or
(af) a HCVR that comprises:
an HCDR1 comprising the amino acid sequence set forth in SEQ ID NO: 313 (or a variant thereof),
an HCDR2 comprising the amino acid sequence set forth in SEQ ID NO: 314 (or a variant thereof), and
an HCDR3 comprising the amino acid sequence set forth in SEQ ID NO: 315 (or a variant thereof); and
a LCVR that comprises:
an LCDR1 comprising the amino acid sequence set forth in SEQ ID NO: 318 (or a variant thereof),
an LCDR2 comprising the amino acid sequence set forth in SEQ ID NO: 319 (or a variant thereof), and
an LCDR3 comprising the amino acid sequence set forth in SEQ ID NO: 320 (or a variant thereof).

18. The protein-drug conjugate of any one of claim 1-17, wherein the antigen-binding protein comprises:
(i) a HCVR that comprises the amino acid sequence set forth in SEQ ID NO: 2 (or a variant thereof); and a LCVR that comprises the amino acid sequence set forth in SEQ ID NO: 7 (or a variant thereof);
(ii) a HCVR that comprises the amino acid sequence set forth in SEQ ID NO: 12 (or a variant thereof); and a LCVR that comprises the amino acid sequence set forth in SEQ ID NO: 17 (or a variant thereof);
(iii) a HCVR that comprises the amino acid sequence set forth in SEQ ID NO: 22 (or a variant thereof); and a LCVR that comprises the amino acid sequence set forth in SEQ ID NO: 27 (or a variant thereof);
(iv) a HCVR that comprises the amino acid sequence set forth in SEQ ID NO: 32 (or a variant thereof); and a LCVR that comprises the amino acid sequence set forth in SEQ ID NO: 37 (or a variant thereof);
(v) a HCVR that comprises the amino acid sequence set forth in SEQ ID NO: 42 (or a variant thereof); and a LCVR that comprises the amino acid sequence set forth in SEQ ID NO: 47 (or a variant thereof);
(vi) a HCVR that comprises the amino acid sequence set forth in SEQ ID NO: 52 (or a variant thereof); and a LCVR that comprises the amino acid sequence set forth in SEQ ID NO: 57 (or a variant thereof);
(vii) a HCVR that comprises the amino acid sequence set forth in SEQ ID NO: 62 (or a variant thereof); and a LCVR that comprises the amino acid sequence set forth in SEQ ID NO: 67 (or a variant thereof);
(viii) a HCVR that comprises the amino acid sequence set forth in SEQ ID NO: 72 (or a variant thereof); and a LCVR that comprises the amino acid sequence set forth in SEQ ID NO: 77 (or a variant thereof);
(ix) a HCVR that comprises the amino acid sequence set forth in SEQ ID NO: 82 (or a variant thereof); and a LCVR that comprises the amino acid sequence set forth in SEQ ID NO: 87 (or a variant thereof);
(x) a HCVR that comprises the amino acid sequence set forth in SEQ ID NO: 92 (or a variant thereof); and a LCVR that comprises the amino acid sequence set forth in SEQ ID NO: 97 (or a variant thereof);
(xi) a HCVR that comprises the amino acid sequence set forth in SEQ ID NO: 102 (or a variant thereof); and a LCVR that comprises the amino acid sequence set forth in SEQ ID NO: 107 (or a variant thereof);
(xii) a HCVR that comprises the amino acid sequence set forth in SEQ ID NO: 112 (or a variant thereof); and a LCVR that comprises the amino acid sequence set forth in SEQ ID NO: 117 (or a variant thereof);
(xiii) a HCVR that comprises the amino acid sequence set forth in SEQ ID NO: 122 (or a variant thereof); and a LCVR that comprises the amino acid sequence set forth in SEQ ID NO: 127 (or a variant thereof);
(xiv) a HCVR that comprises the amino acid sequence set forth in SEQ ID NO: 132 (or a variant thereof); and a LCVR that comprises the amino acid sequence set forth in SEQ ID NO: 137 (or a variant thereof);
(xv) a HCVR that comprises the amino acid sequence set forth in SEQ ID NO: 142 (or a variant thereof); and a LCVR that comprises the amino acid sequence set forth in SEQ ID NO: 147 (or a variant thereof);
(xvi) a HCVR that comprises the amino acid sequence set forth in SEQ ID NO: 152 (or a variant thereof); and a LCVR that comprises the amino acid sequence set forth in SEQ ID NO: 157 (or a variant thereof);
(xvii) a HCVR that comprises the amino acid sequence set forth in SEQ ID NO: 162 (or a variant thereof); and a LCVR that comprises the amino acid sequence set forth in SEQ ID NO: 167 (or a variant thereof);
(xviii) a HCVR that comprises the amino acid sequence set forth in SEQ ID NO: 172 (or a variant thereof); and a LCVR that comprises the amino acid sequence set forth in SEQ ID NO: 177 (or a variant thereof);
(xix) a HCVR that comprises the amino acid sequence set forth in SEQ ID NO: 182 (or a variant thereof); and a LCVR that comprises the amino acid sequence set forth in SEQ ID NO: 187 (or a variant thereof);
(xx) a HCVR that comprises the amino acid sequence set forth in SEQ ID NO: 192 (or a variant thereof); and a LCVR that comprises the amino acid sequence set forth in SEQ ID NO: 197 (or a variant thereof);
(xxi) a HCVR that comprises the amino acid sequence set forth in SEQ ID NO: 202 (or a variant thereof); and a LCVR that comprises the amino acid sequence set forth in SEQ ID NO: 207 (or a variant thereof);
(xxii) a HCVR that comprises the amino acid sequence set forth in SEQ ID NO: 212 (or a variant thereof); and a LCVR that comprises the amino acid sequence set forth in SEQ ID NO: 217 (or a variant thereof);
(xxiii) a HCVR that comprises the amino acid sequence set forth in SEQ ID NO: 222 (or a variant thereof); and a LCVR that comprises the amino acid sequence set forth in SEQ ID NO: 227 (or a variant thereof);
(xxiv) a HCVR that comprises the amino acid sequence set forth in SEQ ID NO: 232 (or a variant thereof); and a LCVR that comprises the amino acid sequence set forth in SEQ ID NO: 237 (or a variant thereof);
(xxv) a HCVR that comprises the amino acid sequence set forth in SEQ ID NO: 242 (or a variant thereof); and a LCVR that comprises the amino acid sequence set forth in SEQ ID NO: 247 (or a variant thereof);
(xxvi) a HCVR that comprises the amino acid sequence set forth in SEQ ID NO: 252 (or a variant thereof); and a LCVR that comprises the amino acid sequence set forth in SEQ ID NO: 257 (or a variant thereof);
(xxvii) a HCVR that comprises the amino acid sequence set forth in SEQ ID NO: 262 (or a variant thereof); and a LCVR that comprises the amino acid sequence set forth in SEQ ID NO: 267 (or a variant thereof);
(xxviii) a HCVR that comprises the amino acid sequence set forth in SEQ ID NO: 272 (or a variant thereof); and a LCVR that comprises the amino acid sequence set forth in SEQ ID NO: 277 (or a variant thereof);
(xxix) a HCVR that comprises the amino acid sequence set forth in SEQ ID NO: 282 (or a variant thereof); and a LCVR that comprises the amino acid sequence set forth in SEQ ID NO: 287 (or a variant thereof);
(xxx) a HCVR that comprises the amino acid sequence set forth in SEQ ID NO: 292 (or a variant thereof); and a LCVR that comprises the amino acid sequence set forth in SEQ ID NO: 297 (or a variant thereof);
(xxxi) a HCVR that comprises the amino acid sequence set forth in SEQ ID NO: 302 (or a variant thereof); and a LCVR that comprises the amino acid sequence set forth in SEQ ID NO: 307 (or a variant thereof); and/or
(xxxii) a HCVR that comprises the amino acid sequence set forth in SEQ ID NO: 312 (or a variant thereof); and a LCVR that comprises the amino acid sequence set forth in SEQ ID NO: 317 (or a variant thereof).

19. The protein-drug conjugate of any one of claim 1-4 and 11-18 wherein the antigen-binding protein comprises:
i. a heavy chain region that comprises the amino acid sequence set forth in SEQ ID NO: 329 (or a variant thereof); and a light chain that comprises the amino acid sequence set forth in SEQ ID NO: 328 (or a variant thereof);
ii. a heavy chain region that comprises the amino acid sequence set forth in SEQ ID NO: 331 (or a variant thereof); and a light chain that comprises the amino acid sequence set forth in SEQ ID NO: 330 (or a variant thereof);
iii. a heavy chain region that comprises the amino acid sequence set forth in SEQ ID NO: 333 (or a variant thereof); and a light chain that comprises the amino acid sequence set forth in SEQ ID NO: 332 (or a variant thereof);
iv. a heavy chain region that comprises the amino acid sequence set forth in SEQ ID NO: 335 (or a variant thereof); and a light chain that comprises the amino acid sequence set forth in SEQ ID NO: 334 (or a variant thereof);
v. a heavy chain region that comprises the amino acid sequence set forth in SEQ ID NO: 337 (or a variant thereof); and a light chain that comprises the amino acid sequence set forth in SEQ ID NO: 336 (or a variant thereof);
vi. a heavy chain region that comprises the amino acid sequence set forth in SEQ ID NO: 339 (or a variant thereof); and a light chain that comprises the amino acid sequence set forth in SEQ ID NO: 338 (or a variant thereof);
vii. a heavy chain region that comprises the amino acid sequence set forth in SEQ ID NO: 341 (or a variant thereof); and a light chain that comprises the amino acid sequence set forth in SEQ ID NO: 340 (or a variant thereof);
viii. a heavy chain region that comprises the amino acid sequence set forth in SEQ ID NO: 343 (or a variant thereof); and a light chain that comprises the amino acid sequence set forth in SEQ ID NO: 342 (or a variant thereof);
ix. a heavy chain region that comprises the amino acid sequence set forth in SEQ ID NO: 345 (or a variant thereof); and a light chain that comprises the amino acid sequence set forth in SEQ ID NO: 344 (or a variant thereof);
x. a heavy chain region that comprises the amino acid sequence set forth in SEQ ID NO: 347 (or a variant thereof); and a light chain that comprises the amino acid sequence set forth in SEQ ID NO: 346 (or a variant thereof);
xi. a heavy chain region that comprises the amino acid sequence set forth in SEQ ID NO: 349 (or a variant thereof); and a light chain that comprises the amino acid sequence set forth in SEQ ID NO: 348 (or a variant thereof);
xii. a heavy chain region that comprises the amino acid sequence set forth in SEQ ID NO: 351 (or a variant thereof); and a light chain that comprises the amino acid sequence set forth in SEQ ID NO: 350 (or a variant thereof);
xiii. a heavy chain region that comprises the amino acid sequence set forth in SEQ ID NO: 353 (or a variant thereof); and a light chain that comprises the amino acid sequence set forth in SEQ ID NO: 352 (or a variant thereof);
xiv. a heavy chain region that comprises the amino acid sequence set forth in SEQ ID NO: 355 (or a variant thereof); and a light chain that comprises the amino acid sequence set forth in SEQ ID NO: 354 (or a variant thereof);
xv. a heavy chain region that comprises the amino acid sequence set forth in SEQ ID NO: 357 (or a variant thereof); and a light chain that comprises the amino acid sequence set forth in SEQ ID NO: 356 (or a variant thereof);
xvi. a heavy chain region that comprises the amino acid sequence set forth in SEQ ID NO: 359 (or a variant thereof); and a light chain that comprises the amino acid sequence set forth in SEQ ID NO: 358 (or a variant thereof);
xvii. a heavy chain region that comprises the amino acid sequence set forth in SEQ ID NO: 361 (or a variant thereof); and a light chain that comprises the amino acid sequence set forth in SEQ ID NO: 360 (or a variant thereof);
xviii. a heavy chain region that comprises the amino acid sequence set forth in SEQ ID NO: 363 (or a variant thereof); and a light chain that comprises the amino acid sequence set forth in SEQ ID NO: 362 (or a variant thereof);
xix. a heavy chain region that comprises the amino acid sequence set forth in SEQ ID NO: 365 (or a variant thereof); and a light chain that comprises the amino acid sequence set forth in SEQ ID NO: 364 (or a variant thereof);
xx. a heavy chain region that comprises the amino acid sequence set forth in SEQ ID NO: 367 (or a variant thereof); and a light chain that comprises the amino acid sequence set forth in SEQ ID NO: 366 (or a variant thereof);
xxi. a heavy chain region that comprises the amino acid sequence set forth in SEQ ID NO: 369 (or a variant thereof); and a light chain that comprises the amino acid sequence set forth in SEQ ID NO: 368 (or a variant thereof);
xxii. a heavy chain region that comprises the amino acid sequence set forth in SEQ ID NO: 371 (or a variant thereof); and a light chain that comprises the amino acid sequence set forth in SEQ ID NO: 370 (or a variant thereof);
xxiii. a heavy chain region that comprises the amino acid sequence set forth in SEQ ID NO: 373 (or a variant thereof); and a light chain that comprises the amino acid sequence set forth in SEQ ID NO: 372 (or a variant thereof);
xxiv. a heavy chain region that comprises the amino acid sequence set forth in SEQ ID NO: 375 (or a variant thereof); and a light chain that comprises the amino acid sequence set forth in SEQ ID NO: 374 (or a variant thereof);
xxv. a heavy chain region that comprises the amino acid sequence set forth in SEQ ID NO: 377 (or a variant thereof); and a light chain that comprises the amino acid sequence set forth in SEQ ID NO: 376 (or a variant thereof);
xxvi. a heavy chain region that comprises the amino acid sequence set forth in SEQ ID NO: 379 (or a variant thereof); and a light chain that comprises the amino acid sequence set forth in SEQ ID NO: 378 (or a variant thereof);
xxvii. a heavy chain region that comprises the amino acid sequence set forth in SEQ ID NO: 381 (or a variant thereof); and a light chain that comprises the amino acid sequence set forth in SEQ ID NO: 380 (or a variant thereof);
xxviii. a heavy chain region that comprises the amino acid sequence set forth in SEQ ID NO: 383 (or a variant thereof); and a light chain that comprises the amino acid sequence set forth in SEQ ID NO: 382 (or a variant thereof);
xxix. a heavy chain region that comprises the amino acid sequence set forth in SEQ ID NO: 385 (or a variant thereof); and a light chain that comprises the amino acid sequence set forth in SEQ ID NO: 384 (or a variant thereof);
xxx. a heavy chain region that comprises the amino acid sequence set forth in SEQ ID NO: 387 (or a variant thereof); and a light chain that comprises the amino acid sequence set forth in SEQ ID NO: 386 (or a variant thereof);
xxxi. a heavy chain region that comprises the amino acid sequence set forth in SEQ ID NO: 389 (or a variant thereof); and a light chain that comprises the amino acid sequence set forth in SEQ ID NO: 388 (or a variant thereof); or
xxxii. a heavy chain region that comprises the amino acid sequence set forth in SEQ ID NO: 391 (or a variant thereof); and a light chain that comprises the amino acid sequence set forth in SEQ ID NO: 390 (or a variant thereof).

20. The protein-drug conjugate of any one of claim 1-3 and 11-23, wherein the antigen-binding protein comprises:
i. a heavy chain that comprises the amino acid sequence set forth in SEQ ID NO: 543 (or a variant thereof); and a light chain that comprises the amino acid sequence set forth in SEQ ID NO: 328 (or a variant thereof);
ii. a heavy chain that comprises the amino acid sequence set forth in SEQ ID NO: 544 (or a variant thereof); and a light chain that comprises the amino acid sequence set forth in SEQ ID NO: 330 (or a variant thereof);
iii. a heavy chain that comprises the amino acid sequence set forth in SEQ ID NO: 545 (or a variant thereof); and a light chain that comprises the amino acid sequence set forth in SEQ ID NO: 332 (or a variant thereof);
iv. a heavy chain that comprises the amino acid sequence set forth in SEQ ID NO: 546 (or a variant thereof); and a light chain that comprises the amino acid sequence set forth in SEQ ID NO: 334 (or a variant thereof);
v. a heavy chain that comprises the amino acid sequence set forth in SEQ ID NO: 547 (or a variant thereof); and a light chain that comprises the amino acid sequence set forth in SEQ ID NO: 336 (or a variant thereof);
vi. a heavy chain that comprises the amino acid sequence set forth in SEQ ID NO: 548 (or a variant thereof); and a light chain that comprises the amino acid sequence set forth in SEQ ID NO: 338 (or a variant thereof);
vii. a heavy chain that comprises the amino acid sequence set forth in SEQ ID NO: 549 (or a variant thereof); and a light chain that comprises the amino acid sequence set forth in SEQ ID NO: 340 (or a variant thereof);
viii. a heavy chain that comprises the amino acid sequence set forth in SEQ ID NO: 550 (or a variant thereof); and a light chain that comprises the amino acid sequence set forth in SEQ ID NO: 342 (or a variant thereof);
ix. a heavy chain that comprises the amino acid sequence set forth in SEQ ID NO: 551 (or a variant thereof); and a light chain that comprises the amino acid sequence set forth in SEQ ID NO: 344 (or a variant thereof);
x. a heavy chain that comprises the amino acid sequence set forth in SEQ ID NO: 552 (or a variant thereof); and a light chain that comprises the amino acid sequence set forth in SEQ ID NO: 346 (or a variant thereof);
xi. a heavy chain that comprises the amino acid sequence set forth in SEQ ID NO: 553 (or a variant thereof); and a light chain that comprises the amino acid sequence set forth in SEQ ID NO: 348 (or a variant thereof);
xii. a heavy chain that comprises the amino acid sequence set forth in SEQ ID NO: 554 (or a variant thereof); and a light chain that comprises the amino acid sequence set forth in SEQ ID NO: 350 (or a variant thereof);
xiii. a heavy chain that comprises the amino acid sequence set forth in SEQ ID NO: 555 (or a variant thereof); and a light chain that comprises the amino acid sequence set forth in SEQ ID NO: 352 (or a variant thereof);
xiv. a heavy chain that comprises the amino acid sequence set forth in SEQ ID NO: 556 (or a variant thereof); and a light chain that comprises the amino acid sequence set forth in SEQ ID NO: 354 (or a variant thereof);
xv. a heavy chain that comprises the amino acid sequence set forth in SEQ ID NO: 557 (or a variant thereof); and a light chain that comprises the amino acid sequence set forth in SEQ ID NO: 356 (or a variant thereof);
xvi. a heavy chain that comprises the amino acid sequence set forth in SEQ ID NO: 558 (or a variant thereof); and a light chain that comprises the amino acid sequence set forth in SEQ ID NO: 358 (or a variant thereof);
xvii. a heavy chain that comprises the amino acid sequence set forth in SEQ ID NO: 559 (or a variant thereof); and a light chain that comprises the amino acid sequence set forth in SEQ ID NO: 360 (or a variant thereof);
xviii. a heavy chain that comprises the amino acid sequence set forth in SEQ ID NO: 560 (or a variant thereof); and a light chain that comprises the amino acid sequence set forth in SEQ ID NO: 362 (or a variant thereof);
xix. a heavy chain that comprises the amino acid sequence set forth in SEQ ID NO: 561 (or a variant thereof); and a light chain that comprises the amino acid sequence set forth in SEQ ID NO: 364 (or a variant thereof);
xx. a heavy chain that comprises the amino acid sequence set forth in SEQ ID NO: 562 (or a variant thereof); and a light chain that comprises the amino acid sequence set forth in SEQ ID NO: 366 (or a variant thereof);
xxi. a heavy chain that comprises the amino acid sequence set forth in SEQ ID NO: 563 (or a variant thereof); and a light chain that comprises the amino acid sequence set forth in SEQ ID NO: 368 (or a variant thereof);
xxii. a heavy chain that comprises the amino acid sequence set forth in SEQ ID NO: 564 (or a variant thereof); and a light chain that comprises the amino acid sequence set forth in SEQ ID NO: 370 (or a variant thereof);
xxiii. a heavy chain that comprises the amino acid sequence set forth in SEQ ID NO: 565 (or a variant thereof); and a light chain that comprises the amino acid sequence set forth in SEQ ID NO: 372 (or a variant thereof);
xxiv. a heavy chain that comprises the amino acid sequence set forth in SEQ ID NO: 566 (or a variant thereof); and a light chain that comprises the amino acid sequence set forth in SEQ ID NO: 374 (or a variant thereof);
xxv. a heavy chain that comprises the amino acid sequence set forth in SEQ ID NO: 567 (or a variant thereof); and a light chain that comprises the amino acid sequence set forth in SEQ ID NO: 376 (or a variant thereof);
xxvi. a heavy chain that comprises the amino acid sequence set forth in SEQ ID NO: 568 (or a variant thereof); and a light chain that comprises the amino acid sequence set forth in SEQ ID NO: 378 (or a variant thereof);
xxvii. a heavy chain that comprises the amino acid sequence set forth in SEQ ID NO: 569 (or a variant thereof); and a light chain that comprises the amino acid sequence set forth in SEQ ID NO: 380 (or a variant thereof);
xxviii. a heavy chain that comprises the amino acid sequence set forth in SEQ ID NO: 570 (or a variant thereof); and a light chain that comprises the amino acid sequence set forth in SEQ ID NO: 382 (or a variant thereof);
xxix. a heavy chain that comprises the amino acid sequence set forth in SEQ ID NO: 571 (or a variant thereof); and a light chain that comprises the amino acid sequence set forth in SEQ ID NO: 384 (or a variant thereof);
xxx. a heavy chain that comprises the amino acid sequence set forth in SEQ ID NO: 572 (or a variant thereof); and a light chain that comprises the amino acid sequence set forth in SEQ ID NO: 386 (or a variant thereof);
xxxi. a heavy chain that comprises the amino acid sequence set forth in SEQ ID NO: 573 (or a variant thereof); and a light chain that comprises the amino acid sequence set forth in SEQ ID NO: 388 (or a variant thereof); or
xxxii. a heavy chain that comprises the amino acid sequence set forth in SEQ ID NO: 574 (or a variant thereof); and a light chain that comprises the amino acid sequence set forth in SEQ ID NO: 390 (or a variant thereof).

21. The protein-drug conjugate of any one of claim 1-16, wherein the antigen-binding protein comprises:
(1) a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof);
(2) a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof);
(3) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof);
(4) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof);
(5) a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof); or
(6) a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof).

22. The protein-drug conjugate of any one of claim 1-17 and 21, wherein the antigen-binding protein comprises:
(a) a HCVR that comprises:
an HCDR1 comprising the amino acid sequence set forth in SEQ ID NO: 133 (or a variant thereof),
an HCDR2 comprising the amino acid sequence set forth in SEQ ID NO: 134 (or a variant thereof), and
an HCDR3 comprising the amino acid sequence set forth in SEQ ID NO: 135 (or a variant thereof); and
a LCVR that comprises:
an LCDR1 comprising the amino acid sequence set forth in SEQ ID NO: 138 (or a variant thereof),
an LCDR2 comprising the amino acid sequence set forth in SEQ ID NO: 139 (or a variant thereof), and
an LCDR3 comprising the amino acid sequence set forth in SEQ ID NO: 140 (or a variant thereof);
(b) a HCVR that comprises:
an HCDR1 comprising the amino acid sequence set forth in SEQ ID NO: 173 (or a variant thereof),
an HCDR2 comprising the amino acid sequence set forth in SEQ ID NO: 174 (or a variant thereof), and
an HCDR3 comprising the amino acid sequence set forth in SEQ ID NO: 175 (or a variant thereof); and
a LCVR that comprises:
an LCDR1 comprising the amino acid sequence set forth in SEQ ID NO: 178 (or a variant thereof),
an LCDR2 comprising the amino acid sequence set forth in SEQ ID NO: 179 (or a variant thereof), and
an LCDR3 comprising the amino acid sequence set forth in SEQ ID NO: 180 (or a variant thereof);
(c) a HCVR that comprises:
an HCDR1 comprising the amino acid sequence set forth in SEQ ID NO: 223 (or a variant thereof),
an HCDR2 comprising the amino acid sequence set forth in SEQ ID NO: 224 (or a variant thereof), and
an HCDR3 comprising the amino acid sequence set forth in SEQ ID NO: 225 (or a variant thereof); and
a LCVR that comprises:
an LCDR1 comprising the amino acid sequence set forth in SEQ ID NO: 228 (or a variant thereof),
an LCDR2 comprising the amino acid sequence set forth in SEQ ID NO: 229 (or a variant thereof), and
an LCDR3 comprising the amino acid sequence set forth in SEQ ID NO: 230 (or a variant thereof);
(d) a HCVR that comprises:
an HCDR1 comprising the amino acid sequence set forth in SEQ ID NO: 243 (or a variant thereof),
an HCDR2 comprising the amino acid sequence set forth in SEQ ID NO: 244 (or a variant thereof), and
an HCDR3 comprising the amino acid sequence set forth in SEQ ID NO: 245 (or a variant thereof); and
a LCVR that comprises:
an LCDR1 comprising the amino acid sequence set forth in SEQ ID NO: 248 (or a variant thereof),
an LCDR2 comprising the amino acid sequence set forth in SEQ ID NO: 249 (or a variant thereof), and
an LCDR3 comprising the amino acid sequence set forth in SEQ ID NO: 250 (or a variant thereof);
(e) a HCVR that comprises:
an HCDR1 comprising the amino acid sequence set forth in SEQ ID NO: 263 (or a variant thereof),
an HCDR2 comprising the amino acid sequence set forth in SEQ ID NO: 264 (or a variant thereof), and
an HCDR3 comprising the amino acid sequence set forth in SEQ ID NO: 265 (or a variant thereof); and
a LCVR that comprises:
an LCDR1 comprising the amino acid sequence set forth in SEQ ID NO: 268 (or a variant thereof),
an LCDR2 comprising the amino acid sequence set forth in SEQ ID NO: 269 (or a variant thereof), and
an LCDR3 comprising the amino acid sequence set forth in SEQ ID NO: 270 (or a variant thereof); or
(f) a HCVR that comprises:
an HCDR1 comprising the amino acid sequence set forth in SEQ ID NO: 273 (or a variant thereof),
an HCDR2 comprising the amino acid sequence set forth in SEQ ID NO: 274 (or a variant thereof), and
an HCDR3 comprising the amino acid sequence set forth in SEQ ID NO: 275 (or a variant thereof); and
a LCVR that comprises:
an LCDR1 comprising the amino acid sequence set forth in SEQ ID NO: 278 (or a variant thereof),
an LCDR2 comprising the amino acid sequence set forth in SEQ ID NO: 279 (or a variant thereof), and
an LCDR3 comprising the amino acid sequence set forth in SEQ ID NO: 280 (or a variant thereof).

23. The protein-drug conjugate of any one of claim 1-18 and 21-22 which comprises:
(i) a HCVR that comprises the amino acid sequence set forth in SEQ ID NO: 132 (or a variant thereof); and a LCVR that comprises the amino acid sequence set forth in SEQ ID NO: 137 (or a variant thereof);
(ii) a HCVR that comprises the amino acid sequence set forth in SEQ ID NO: 172 (or a variant thereof); and a LCVR that comprises the amino acid sequence set forth in SEQ ID NO: 177 (or a variant thereof);
(iii) a HCVR that comprises the amino acid sequence set forth in SEQ ID NO: 222 (or a variant thereof); and a LCVR that comprises the amino acid sequence set forth in SEQ ID NO: 227 (or a variant thereof);
(iv) a HCVR that comprises the amino acid sequence set forth in SEQ ID NO: 242 (or a variant thereof); and a LCVR that comprises the amino acid sequence set forth in SEQ ID NO: 247 (or a variant thereof);
(v) a HCVR that comprises the amino acid sequence set forth in SEQ ID NO: 262 (or a variant thereof); and a LCVR that comprises the amino acid sequence set forth in SEQ ID NO: 267 (or a variant thereof); or
(vi) a HCVR that comprises the amino acid sequence set forth in SEQ ID NO: 272 (or a variant thereof); and a LCVR that comprises the amino acid sequence set forth in SEQ ID NO: 277 (or a variant thereof).

24. The protein-drug conjugate of any one of claims 1-4 and 11-23 wherein the antigen-binding protein comprises:
(A) a heavy chain region that comprises the amino acid sequence set forth in SEQ ID NO: 355 (or a variant thereof); and a light chain that comprises the amino acid sequence set forth in SEQ ID NO: 354 (or a variant thereof);
(B) a heavy chain region that comprises the amino acid sequence set forth in SEQ ID NO: 363 (or a variant thereof); and a light chain that comprises the amino acid sequence set forth in SEQ ID NO: 362 (or a variant thereof);
(C) a heavy chain region that comprises the amino acid sequence set forth in SEQ ID NO: 373 (or a variant thereof); and a light chain that comprises the amino acid sequence set forth in SEQ ID NO: 372 (or a variant thereof);
(D) a heavy chain region that comprises the amino acid sequence set forth in SEQ ID NO: 377 (or a variant thereof); and a light chain that comprises the amino acid sequence set forth in SEQ ID NO: 376 (or a variant thereof);
(E) a heavy chain region that comprises the amino acid sequence set forth in SEQ ID NO: 381 (or a variant thereof); and a light chain that comprises the amino acid sequence set forth in SEQ ID NO: 380 (or a variant thereof); or
(F) a heavy chain region that comprises the amino acid sequence set forth in SEQ ID NO: 383 (or a variant thereof); and a light chain that comprises the amino acid sequence set forth in SEQ ID NO: 382 (or a variant thereof).

25. The protein-drug conjugate of any one of claims 1-4 and 11-24 wherein the antigen-binding protein comprises:
(I) a heavy chain that comprises the amino acid sequence set forth in SEQ ID NO: 556 (or a variant thereof); and a light chain that comprises the amino acid sequence set forth in SEQ ID NO: 354 (or a variant thereof);
(II) a heavy chain that comprises the amino acid sequence set forth in SEQ ID NO: 560 (or a variant thereof); and a light chain that comprises the amino acid sequence set forth in SEQ ID NO: 362 (or a variant thereof);
(Ill) a heavy chain that comprises the amino acid sequence set forth in SEQ ID NO: 565 (or a variant thereof); and a light chain that comprises the amino acid sequence set forth in SEQ ID NO: 372 (or a variant thereof);
(IV) a heavy chain that comprises the amino acid sequence set forth in SEQ ID NO: 367 (or a variant thereof); and a light chain that comprises the amino acid sequence set forth in SEQ ID NO: 376 (or a variant thereof);
(V) a heavy chain that comprises the amino acid sequence set forth in SEQ ID NO: 569 (or a variant thereof); and a light chain that comprises the amino acid sequence set forth in SEQ ID NO: 380 (or a variant thereof); or
(VI) a heavy chain that comprises the amino acid sequence set forth in SEQ ID NO: 570 (or a variant thereof); and a light chain that comprises the amino acid sequence set forth in SEQ ID NO: 382 (or a variant thereof).

26. The protein-drug conjugate of any one of claim 1-25, wherein the antigen-binding protein binds to the same epitope on human transferrin receptor as an antibody comprising an HCVR/LCVR amino acid sequence pair as set forth in Table 1-1.

27. The protein-drug conjugate of any one of claim 1-25, wherein the antigen-binding protein competes for binding to human transferrin receptor with an antibody comprising an HCVR/LCVR amino acid sequence pair as set forth in Table 1-1.

28. A protein-drug conjugate comprising an antigen-binding protein that binds specifically to human transferrin receptor (hTfR), wherein the antigen-binding protein is conjugated to a molecular cargo and comprises an antibody or antigen-binding fragment thereof, wherein the antibody or antigen-binding fragment thereof binds to one or more epitopes of hTfR selected from:
a. an epitope comprising the sequence LLNE (SEQ ID NO: 529) and/or an epitope comprising the sequence TYKEL (SEQ ID NO: 509);
b. an epitope comprising the sequence DSTDFTGT (SEQ ID NO: 530) and/or an epitope comprising the sequence VKHPVTGQF (SEQ ID NO: 531) and/or an epitope comprising the sequence IERIPEL (SEQ ID NO: 532);
c. an epitope comprising the sequence LNENSYVPREAGSQKDEN (SEQ ID NO: 533);
d. an epitope comprising the sequence FEDL (SEQ ID NO: 521);
e. an epitope comprising the sequence IVDKNGRL (SEQ ID NO: 534);
f. an epitope comprising the sequence IVDKNGRLVY (SEQ ID NO: 535);
g. an epitope comprising the sequence DQTKF (SEQ ID NO: 536);
h. an epitope comprising the sequence LVENPGGY (SEQ ID NO: 537) and/or an epitope comprising the sequence PIVNAELSF (SEQ ID NO: 538) and/or an epitope comprising the sequence PYLGTTMDT (SEQ ID NO: 539);
i. an epitope comprising the sequence LLNENSYVPREAGSQKDENLAL (SEQ ID NO: 507) and/or an epitope comprising the sequence IYMDQTKFPIVNAEL (SEQ ID NO: 508) and/or an epitope comprising the sequence TYKEL (SEQ ID NO: 509);
j. an epitope comprising the sequence KRKLSEKLDSTDFTGTIKL (SEQ ID NO: 510) and/or an epitope comprising the sequence YTLIEKTMQNVKHPVTGQFL (SEQ ID NO: 511) and/or an epitope comprising the sequence LIERIPELNKVARAAAE (SEQ ID NO: 512);
k. an epitope comprising the sequence LNENSYVPREAGSQKDENL (SEQ ID NO: 513);
l. an epitope comprising the sequence GTKKDFEDL (SEQ ID NO: 514);
m. an epitope comprising the sequence SVIIVDKNGRLVYLVENPGGYVAYSK (SEQ ID NO: 515);
n. an epitope comprising the sequence LLNENSYVPREAGSQKDEN (SEQ ID NO: 516) and/or an epitope comprising the sequence DQTKFPIVNAEL (SEQ ID NO: 517) and/or an epitope comprising the sequence TYKELIERIPELNK (SEQ ID NO: 518);
o. an epitope comprising the sequence LLNENSYVPREAGSQKDEN (SEQ ID NO: 516) and/or an epitope comprising the sequence TYKELIERIPELNK (SEQ ID NO: 518);
p. an epitope comprising the sequence SVIIVDKNGRLVYLVENPGGYVAY (SEQ ID NO: 519);
q. an epitope comprising the sequence IYMDQTKFPIVNAEL (SEQ ID NO: 508) and/or an epitope comprising the sequence FGNMEGDCPSDWKTDSTCRM (SEQ ID NO: 520);
r. an epitope comprising the sequence LLNENSYVPREAGSQKDENLAL (SEQ ID NO: 507) and/or an epitope comprising the sequence LVENPGGYVAYSKAATVTGKL (SEQ ID NO: 522) and/or an epitope comprising the sequence IYMDQTKFPIVNAELSF (SEQ ID NO: 523) and/or an epitope comprising the sequence ISRAAAEKL (SEQ ID NO: 524) and/or an epitope comprising the sequence VTSESKNVKLTVSNVLKE (SEQ ID NO: 525) and/or an epitope comprising the sequence FCEDTDYPYLGTTMDT (SEQ ID NO: 526);
s. an epitope comprised within or overlapping with the sequence LLNENSYVPREAGSQKDENLAL (SEQ ID NO: 507) and/or an epitope comprised within or overlapping with the sequence IYMDQTKFPIVNAEL (SEQ ID NO: 508) and/or an epitope comprised within or overlapping with the sequence TYKEL (SEQ ID NO: 509);
t. an epitope comprised within or overlapping with the sequence KRKLSEKLDSTDFTGTIKL (SEQ ID NO: 510) and/or an epitope comprised within or overlapping with the sequence YTLIEKTMQNVKHPVTGQFL (SEQ ID NO: 511) and/or an epitope comprised within or overlapping with the sequence LIERIPELNKVARAAAE (SEQ ID NO: 512);
u. an epitope comprised within or overlapping with the sequence LNENSYVPREAGSQKDENL (SEQ ID NO: 513);
v. an epitope comprised within or overlapping with the sequence GTKKDFEDL (SEQ ID NO: 514);
w. an epitope comprised within or overlapping with the sequence SVIIVDKNGRLVYLVENPGGYVAYSK (SEQ ID NO: 515);
x. an epitope comprised within or overlapping with the sequence LLNENSYVPREAGSQKDEN (SEQ ID NO: 516) and/or an epitope comprised within or overlapping with the sequence DQTKFPIVNAEL (SEQ ID NO: 517) and/or an epitope comprised within or overlapping with the sequence TYKELIERIPELNK (SEQ ID NO: 518);
y. an epitope comprised within or overlapping with the sequence LLNENSYVPREAGSQKDEN (SEQ ID NO: 516) and/or an epitope comprised within or overlapping with the sequence TYKELIERIPELNK (SEQ ID NO: 518);
z. an epitope comprised within or overlapping with the sequence SVIIVDKNGRLVYLVENPGGYVAY (SEQ ID NO: 519;
aa. an epitope comprised within or overlapping with the sequence IYMDQTKFPIVNAEL (SEQ ID NO: 508) and/or an epitope comprised within or overlapping with the sequence FGNMEGDCPSDWKTDSTCRM (SEQ ID NO: 520); and
bb. an epitope comprised within or overlapping with the sequence LLNENSYVPREAGSQKDENLAL (SEQ ID NO: 507) and/or an epitope comprised within or overlapping with the sequence LVENPGGYVAYSKAATVTGKL (SEQ ID NO: 522) and/or an epitope comprised within or overlapping with the sequence IYMDQTKFPIVNAELSF (SEQ ID NO: 523) and/or an epitope comprised within or overlapping with the sequence ISRAAAEKL (SEQ ID NO: 524) and/or an epitope comprised within or overlapping with the sequence VTSESKNVKLTVSNVLKE (SEQ ID NO: 525) and/or an epitope comprised within or overlapping with the sequence FCEDTDYPYLGTTMDT (SEQ ID NO: 526).

29. The protein-drug conjugate of claim 28, wherein the antibody or antigen-binding fragment thereof binds to one or more epitopes of hTfR selected from:
a. an epitope consisting of the sequence LLNE (SEQ ID NO: 529) and/or an epitope consisting of the sequence TYKEL (SEQ ID NO: 509);
b. an epitope consisting of the sequence DSTDFTGT (SEQ ID NO: 530) and/or an epitope consisting of the sequence VKHPVTGQF (SEQ ID NO: 531) and/or an epitope consisting of the sequence IERIPEL (SEQ ID NO: 532);
c. an epitope consisting of the sequence LNENSYVPREAGSQKDEN (SEQ ID NO: 533);
d. an epitope consisting of the sequence FEDL (SEQ ID NO: 521);
e. an epitope consisting of the sequence IVDKNGRL (SEQ ID NO: 534);
f. an epitope consisting of the sequence IVDKNGRLVY (SEQ ID NO: 535);
g. an epitope consisting of the sequence DQTKF (SEQ ID NO: 536);
h. an epitope consisting of the sequence LVENPGGY (SEQ ID NO: 537) and/or an epitope consisting of the sequence PIVNAELSF (SEQ ID NO: 538) and/or an epitope consisting of the sequence PYLGTTMDT (SEQ ID NO: 539);
i. an epitope consisting of the sequence LLNENSYVPREAGSQKDENLAL (SEQ ID NO: 507) and/or an epitope consisting of the sequence IYMDQTKFPIVNAEL (SEQ ID NO: 508) and/or an epitope consisting of the sequence TYKEL (SEQ ID NO: 509);
j. an epitope consisting of the sequence KRKLSEKLDSTDFTGTIKL (SEQ ID NO: 510) and/or an epitope consisting of the sequence YTLIEKTMQNVKHPVTGQFL (SEQ ID NO: 511) and/or an epitope consisting of the sequence LIERIPELNKVARAAAE (SEQ ID NO: 512);
k. an epitope consisting of the sequence LNENSYVPREAGSQKDENL (SEQ ID NO: 513);
l. an epitope consisting of the sequence GTKKDFEDL (SEQ ID NO: 514);
m. an epitope consisting of the sequence SVIIVDKNGRLVYLVENPGGYVAYSK (SEQ ID NO: 515);
n. an epitope consisting of the sequence LLNENSYVPREAGSQKDEN (SEQ ID NO: 516) and/or an epitope consisting of the sequence DQTKFPIVNAEL (SEQ ID NO: 517) and/or an epitope consisting of the sequence TYKELIERIPELNK (SEQ ID NO: 518);
o. an epitope consisting of the sequence LLNENSYVPREAGSQKDEN (SEQ ID NO: 516) and/or an epitope consisting of the sequence TYKELIERIPELNK (SEQ ID NO: 518);
p. an epitope consisting of the sequence SVIIVDKNGRLVYLVENPGGYVAY (SEQ ID NO: 519);
q. an epitope consisting of the sequence IYMDQTKFPIVNAEL (SEQ ID NO: 508) and/or an epitope consisting of the sequence FGNMEGDCPSDWKTDSTCRM (SEQ ID NO: 520); and
r. an epitope consisting of the sequence LLNENSYVPREAGSQKDENLAL (SEQ ID NO: 507) and/or an epitope consisting of the sequence LVENPGGYVAYSKAATVTGKL (SEQ ID NO: 522) and/or an epitope consisting of the sequence IYMDQTKFPIVNAELSF (SEQ ID NO: 523) and/or an epitope consisting of the sequence ISRAAAEKL (SEQ ID NO: 524) and/or an epitope consisting of the sequence VTSESKNVKLTVSNVLKE (SEQ ID NO: 525) and/or an epitope consisting of the sequence FCEDTDYPYLGTTMDT (SEQ ID NO: 526).

30. The protein-drug conjugate of claim 28 or claim 29, wherein the antigen-binding protein is selected from a humanized antibody or antigen binding fragment thereof, human antibody or antigen binding fragment thereof, murine antibody or antigen binding fragment thereof, chimeric antibody or antigen binding fragment thereof, monovalent Fab′, divalent Fab2, F(ab)′3 fragments, single-chain fragment variable (scFv), bis-scFv, (scFv)2, diabody, bivalent antibody, one-armed antibody, minibody, nanobody, triabody, tetrabody, disulfide stabilized Fv protein (dsFv), single-domain antibody (sdAb), Ig NAR, single heavy chain antibody, bispecific antibody or biding fragment thereof, bi-specific T-cell engager (BiTE), trispecific antibody, or chemically modified derivatives thereof.

31. The protein-drug conjugate of any one of claim 1-30, wherein said molecular cargo is conjugated to:
(i) a HCVR of the antigen-binding protein,
(ii) a LCVR of the antigen-binding protein,
(iii) a heavy chain of the antigen-binding protein, and/or
(iv) a light chain of the antigen-binding protein.

32. The protein-drug conjugate of any one of claim 1-31, wherein said molecular cargo is conjugated to antigen-binding protein via a glutamine residue and/or a lysine residue.

33. The protein-drug conjugate of claim 32, wherein the glutamine residue is:
(i) introduced to the N-terminus and/or C-terminus of a heavy chain of the antigen-binding protein,
(ii) introduced to the N-terminus and/or C-terminus of a light chain of the antigen-binding protein,
(iii) naturally present in a CH2 or CH3 domain of the antigen-binding protein,
(iv) introduced to the antigen-binding protein by modifying one or more amino acids, and/or
(v) Q295 or mutated from N297 to Q297 (N297Q).

34. The protein-drug conjugate of claim 32 or 33, wherein the antigen-binding protein comprises a glutamine-containing tag, and the molecular cargo is conjugated to the antigen-binding protein via a glutamine residue of the glutamine-containing tag.

35. The protein-drug conjugate of claim 34, wherein the glutamine-containing tag comprises an amino acid sequence selected from the group consisting of LLQGG (SEQ ID NO: 439), LLQG (SEQ ID NO: 440), LSLSQG (SEQ ID NO: 441), gGGLLQGG (SEQ ID NO: 442), gLLQG (SEQ ID NO: 443), LLQ (SEQ ID NO: 444), gSPLAQSHGG (SEQ ID NO: 445), gLLQGGG (SEQ ID NO: 446), gLLQGG (SEQ ID NO: 447), gLLQ (SEQ ID NO: 448), LLQLLQGA (SEQ ID NO: 449), LLQGA (SEQ ID NO: 450), LLQYQGA (SEQ ID NO: 451), LLQGSG (SEQ ID NO: 452), LLQYQG (SEQ ID NO: 453), LLQLLQG (SEQ ID NO: 454), SLLQG (SEQ ID NO: 455), LLQLQ (SEQ ID NO: 456), LLQLLQ (SEQ ID NO: 457), and LLQGR (SEQ ID NO: 458).

36. The protein-drug conjugate of any one of claim 1-35 wherein said antigen-binding protein and said molecular cargo are conjugated via a linker.

37. The protein-drug conjugate of any one of claim 1-36, wherein the molecular cargo comprises a polynucleotide molecule, a carrier, or a small molecule.

38. The protein-drug conjugate of any one of claim 1-37, wherein the molecular cargo comprises a polynucleotide molecule.

39. The protein-drug conjugate of claim 38, wherein the polynucleotide molecule is an interfering nucleic acid molecule, a guide RNA, a ribozyme, an aptamer, a mixmer, a multimer, or an mRNA.

40. The protein-drug conjugate of claim 39, wherein the interfering nucleic acid molecule is an siRNA, an shRNA, a miRNA, an antisense oligonucleotide, or a gapmer.

41. The protein-drug conjugate of claim 40, wherein the interfering nucleic acid is an siRNA.

42. The protein-drug conjugate of claim 40, wherein the siRNA inhibits the DMPK, CNBP, Dystrophin, DUX4, ApoE, MAPT, APP, HTT, SOD1, C9orf72, SNCA, LRRK2, PRNP, SLC5A1, SLC16A3, HDAC6, MMP27, MFAP5, FAM64A, BAIAP3, MYH7, TPM1, RBM20, KLHL24, MYL2, or TNNT2 gene or a mutant thereof.

43. The protein-drug conjugate of claim 41 or 42, wherein the siRNA comprises a sense strand of 21 nucleotides in length.

44. The protein-drug conjugate of any one of claims 41-43, wherein the siRNA comprises an antisense strand of 23 nucleotides in length.

45. The protein-drug conjugate of any one of claims 41-44, wherein the siRNA comprises two phosphorothioate linkages at the first and second internucleoside linkages at the 5′ end of the sense strand.

46. The protein-drug conjugate of any one of claims 41-45, wherein the siRNA comprises two phosphorothioate linkages at the first and second internucleoside linkages at the 3′ and/or 5′ ends of the antisense strand.

47. The protein-drug conjugate of claim 40, wherein the interfering nucleic acid is an antisense oligonucleotide.

48. The protein-drug conjugate of claim 39, wherein the polynucleotide molecule is a guide RNA.

49. The protein-drug conjugate of any one of claims 39-48, wherein the polynucleotide molecule comprises one or more modified nucleotides.

50. The protein-drug conjugate of any one of claim 1-37, wherein the molecular cargo comprises a carrier.

51. The protein-drug conjugate of claim 50, wherein the molecular cargo comprises a lipid-based carrier.

52. The protein-drug conjugate of claim 51, wherein the lipid-based carrier is a lipid nanoparticle (LNP), a liposome, a lipidoid, or a lipoplex.

53. The protein-drug conjugate of claim 52, wherein the lipid-based carrier is a LNP.

54. The protein-drug conjugate of claim 53, wherein the LNP further comprises a polynucleotide molecule and/or a polypeptide molecule.

55. The protein-drug conjugate of claim 53 or 54, wherein the LNP comprises one or more components of a gene editing system.

56. The protein-drug conjugate of claim 55, wherein LNP comprises (a) a Cas nuclease, or a nucleic acid encoding the Cas nuclease, and/or (b) a guide RNA, or one or more DNAs encoding the guide RNA.

57. The protein-drug conjugate of claim 56, wherein the Cas nuclease is a Cas9 protein.

58. The protein-drug conjugate of claim 57, wherein the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

59. The protein-drug conjugate of any one of claims 56-58, wherein the nucleic acid encoding the Cas protein is codon-optimized for expression in a mammalian cell.

60. The protein-drug conjugate of claim 59, wherein the nucleic acid encoding the Cas protein is codon-optimized for expression in a human cell.

61. The protein-drug conjugate of any one of claims 56-60, wherein the nucleic acid encoding the Cas nuclease comprises an mRNA.

62. The protein-drug conjugate of any one of claims 39 and 56-61, wherein the guide RNA is a single guide RNA (sgRNA).

63. The protein-drug conjugate of claim 55, wherein the LNP comprises a zinc finger nuclease (ZFN), or a transcription activator-like effector nuclease (TALEN).

64. The protein-drug conjugate of any one of claims 53-63, wherein the lipid nanoparticle comprises a cationic lipid, a neutral lipid, a helper lipid, and a stealth lipid.

65. The protein-drug conjugate of claim 64, wherein the neutral lipid is distearoylphosphatidylcholine (DSPC).

66. The protein-drug conjugate of any one of claims 64-65, wherein the helper lipid is cholesterol.

67. The protein-drug conjugate of any one of claims 64-66, wherein the stealth lipid is PEG2k-DMG.

68. The protein-drug conjugate of any one of claim 1-67 wherein the antigen-binding protein, when not conjugated to a molecular cargo, does not block more than 50% of binding of a human transferrin receptor C-terminal fragment to human holo-transferrin that occurs in the absence of such antigen-binding protein.

69. The protein-drug conjugate of claim 68 wherein said blocking is as measured in an Enzyme Linked Immunosorbent Assay (ELISA) plate assay wherein human transferrin receptor extracellular domain that is fused to a His6-myc-myc tag is pre-bound to said antigen-binding protein and is then contacted with holo-transferrin which is immobilized to the surface of the plate by binding of an anti-holo-transferrin antibody that is bound to the plate.

70. The protein-drug conjugate of any one of claims 68-70 wherein binding of the holo-transferrin and human transferrin receptor extracellular domain in the absence of the antigen-binding protein is measured at a concentration of about 300 pM human transferrin receptor extracellular domain.

71. The protein-drug conjugate of any one of claim 1-70, wherein the antigen-binding protein has one or more of the following characteristics:
a. Affinity (K_{D}) for binding to monkey TfR at 25° C. in surface plasmon resonance format of about 0 nM (no detectable binding) or a higher affinity;
b. Ratio of [K_{D} for binding to monkey TfR/K_{D} for binding to human TfR] at 25° C. in surface plasmon resonance format of from 0 to 278;
c. Blocks about 3-13% hTfR binding to Human Holo-Tf when in Fab format (IgG1);
d. Blocks about 6-13% hTfR binding to Human Holo-Tf when in scFv (V_{K}—V_{H}) format; and/or
e. Blocks about 11-26% hTfR binding to Human Holo-Tf when in scFv (V_{H}-V_{L}) format.

72. A pharmaceutical composition comprising the protein-drug conjugate of any one of claims 1-70 and a pharmaceutically acceptable carrier.

73. A composition or kit comprising the protein-drug conjugate or pharmaceutical composition thereof of any one of claim 1-70 in association with a further therapeutic agent.

74. The composition or kit of claim 73 wherein the further therapeutic agent is selected from: alglucosidase alfa, rituximab, methotrexate, Intravenous immunoglobulin (IVIG), avalglucosidase alfa, levalbuterol, an antibiotic, cortisone, prednisone, a bisphosphonate, and palivizumab.

75. The composition or kit of claim 73 or 74 wherein the further therapeutic agent is selected from: a Beta2-adrenergic agonist, a steroid, a bisphosphonate, an infectious disease treatment, a vaccine, and a Pneumococcal vaccine.

76. A complex comprising the protein-drug conjugate of any one of claim 1-70 bound to a human transferrin receptor polypeptide or a fragment thereof.

77. A method for making a protein-drug conjugate of any one of claim 1-70 comprising
(a) contacting the antigen-binding protein, with the molecular cargo under the conditions favorable for conjugation of the antigen-binding protein to the molecular cargo, and
(b) optionally, isolating the protein-drug conjugate produced in step (a).

78. A protein-drug conjugate which is the product of a method of claim 77.

79. A vessel or injection device comprising the protein-drug conjugate of any one of claims 1-70 and 78.

80. A method for administering a protein-drug conjugate of any one of claim 1-70 and 78 to a subject comprising introducing the protein-drug conjugate into the body of the subject.

81. The method of claim 80 wherein said protein-drug conjugate is introduced into the body of the subject parenterally.

82. The method of claim 81 wherein said protein-drug conjugate is introduced into the body of the subject intravenously.

83. The method of claim 80 wherein said protein-drug conjugate is introduced into the body of the subject via intrathecal, intracerebroventricular, or intraparenchymal injection into the central nervous system.

84. A method for treating or preventing a disease or disorder in a subject in need thereof comprising administering, to the subject, an effective amount of the protein-drug conjugate of any one of claim 1-70 and 78.

85. The method of claim 84, wherein the disease or disorder is a lysosomal storage disease and disorder, a heart disease or disorder, a central nervous system (CNS) disease or disorder, an eye disease or disorder, a brain disease or disorder, a spinal cord disease or disorder, a peripheral nervous system (PNS) disease or disorder, a muscular disease or disorder, a cartilage disease or disorder, a bone growth plate disease or disorder, a kidney disease or disorder, or a blood disease or disorder.

86. The method of claim 84 wherein the disease or disorder is a neurological disease or disorder.

87. The method of claim 86 wherein the neurological disease or disorder is lysosomal storage disease, amyloidosis, neuropathy, neurodegenerative disease, seizure, behavioral disorder, leukodystrophy, neuropsychiatric diseases, traumatic brain injury, neurodevelopmental diseases, neuromuscular diseases, ocular disease or disorder, viral or microbial infection, inflammation, ischemia, and cancer.

88. The method of any one of claims 84-87 wherein the disease or disorder is lysosomal storage disease.

89. The method of claim 87 wherein the neurodegenerative disease is Alzheimer's disease, Huntington's disease, amyotrophic lateral sclerosis, Parkinson's disease, or prion disease.

90. The method of claim 89 wherein the molecular cargo of the protein-drug conjugate is an siRNA selected from the group consisting of an siRNA that inhibits the ApoE, MAPT, APP, HTT, SOD1, C9orf72, SNCA, LRRK2, or PRNP gene or a mutant thereof.

91. The method of claim 85 wherein the disease or disorder is a heart disease or disorder.

92. The method of claim 91 wherein the heart disease or disorder is heart failure.

93. The method of claim 92 wherein the molecular cargo of the protein-drug conjugate is an siRNA selected from the group consisting of an siRNA that inhibits the SLC5A1, SLC16A3, HDAC6, MMP27, MFAP5, FAM64A, BAIAP3, MYH7, TPM1, RBM20, KLHL24, MYL2, or TNNT2 gene or a mutant thereof.

94. The method of claim 85 wherein the disease or disorder is a muscular disease or disorder.

95. The method of claim 94 wherein the muscular disease or disorder is myotonic dystrophy, duchenne muscular dystrophy, fascioscapulohumeral muscular dystrophy, facioscapulohumeral muscular dystrophy-type 1, or muscle atrophy.

96. The method of claim 95 wherein the molecular cargo of the protein-drug conjugate is an siRNA selected from the group consisting of an siRNA that inhibits the DMPK, CNBP, Dystrophin, or DUX4 gene or a mutant thereof.

97. The method of any one of claims 84-96 wherein the subject is administered the protein-drug conjugate in association with a further therapeutic agent.

98. The method of claim 97 wherein the further therapeutic agent is selected from: alglucosidase alfa, rituximab, methotrexate, Intravenous immunoglobulin (IVIG), avalglucosidase alfa, levalbuterol, an antibiotic, cortisone, prednisone, a bisphosphonate, and palivizumab.

99. The method of claim 97 wherein the further therapeutic agent is selected from: a Beta2-adrenergic agonist, a steroid, a bisphosphonate, an infectious disease treatment, a vaccine, and a Pneumococcal vaccine.

100. A method for delivering a molecular cargo to a tissue or cell type in the body of a subject comprising administering, to the subject, an antigen-binding protein that binds specifically to human transferrin receptor or an antigenic-fragment thereof or variant thereof to the subject conjugated to the molecular cargo.

101. The method of claim 100 wherein the molecular cargo comprises a polynucleotide molecule, a carrier or a small molecule.

102. The method of any one of claims 100-101 wherein the tissue is brain/spinal cord/CNS; eye; skeletal muscle; adipose tissue; blood/bone marrow; breast; lung/bronchus; colon; uterus; esophagus; heart; kidney; liver; lymph node; ovary; pancreas; placenta; prostate; rectum; skin; peripheral blood mononuclear cell (PBMC); small intestine; spleen; stomach; testis; peripheral nervous system; and/or bone/cartilage/joint.

103. The method of any one of claims 100-102 wherein the cell type and tissue that is associate with the cell type is as follows:

| | |
|---|---|
| (1) Brain/Spinal cord/CNS tissue | Endothelial cells |
| | Neurons (all types) |
| | Oligodendrocytes (and/or precursors) |
| | Pericytes |
| | Meninges/leptomeningeal cells |
| | Arachnoid barrier cells |
| | Peripheral glia |
| | Astrocytes |
| | Glia |
| | Schwann cells |
| | Ependymal cells |
| | Microglia; |
| (2) Eye tissue | Rod photoreceptor cells |
| | Muller glia cells |
| | Bipolar cells |
| | Cone photoreceptor cells |
| | Endothelial cells |
| | Cornea |
| | Sclera |
| | Optic nerve |
| | Pupillary sphincter; |
| (3) Skeletal Muscle tissue | Skeletal myocytes |
| | Fibroblasts |
| | Endothelial cells |
| | Macrophages |
| | Satellite cells; |
| (4) Adipose tissue | Adipocytes |
| | Fibroblasts |
| | T-cells |
| | Macrophages |
| | B-cells |
| | Dendritic cells; |
| (5) Blood/Bone marrow tissue | T-cells |
| | B-cells |
| | Macrophages |
| | Erythroid cells |
| | Plasmid cells |
| | Dendritic cells; |
| (6) Breast tissue | Glandular cells |
| | T-cells |
| | Fibroblasts |
| | Macrophages |
| | Endothelial cells |
| | Myoepithelial cells |
| | Adipocytes; |
| (7) Lung/Bronchus tissue | Basal respiratory cells |
| | Respiratory cilliated cells |
| | Club cells |
| | Smooth muscle cells |
| | Ionocytes |
| | Macrophages |
| | Alveolar cells (type 1 and/or 2) |
| | T-cells |
| | Endothelial cells; |
| (8) Colon tissue | Distal enterocytes |
| | Intestinal goblet cells |
| | Undifferentiated cells |
| | T-cells |
| | Paneth cells |
| | B-cells |
| | Enteroednocrine cells; |
| (9) Uterus tissue | Glandular and luminal cells |
| | Endometrial stromal cells |
| | Endothelial cells |
| | Smooth muscle cells |
| | T-cells |
| | Macrophages; |
| (10) Esophagus tissue | Fibroblasts |
| | Squamous epithelial cells |
| | Endothelial cells |
| | Smooth muscle cells |
| | Macrophages |
| | Plasma cells |
| | T-cells; |
| (11) Heart tissue | Cardiomyocytes |
| | Endothelial cells |
| | Fibroblasts |
| | Macrophages |
| | T-cells |
| | B-cells |
| | Dendritic cells; |
| (12) Kidney tissue | Proximal tubular cells |
| | T-cells |
| | Macrophages |
| | Collecting duct cells |
| | B-cells |
| | Glomeruli |
| | Fibroblasts; |
| (13) Liver tissue | Hepatocytes |
| | B-cells |
| | Erythroid cells; |
| (14) Lymph node tissue | B-cells |
| | T-cells; |
| (15) Ovary tissue | Granulosa cells |
| | Fibroblasts |
| | Smooth muscle cells |
| | Macrophages |
| | T-cells |
| | Theca cells |
| | Fibroblasts; |
| (16) Pancreas tissue | Ductal cells |
| | Pancreatic endocrine cells |
| | Smooth muscle cells |
| | Endothelial cells |
| | Macrophages |
| | Exocrine glandular cells |
| | Monocytes; |
| (17) Placenta tissue | Cytotrophoblasts |
| | Extravillous trophoblasts |
| | Fibroblasts |
| | Hofbauer cells |
| | Endothelial cells; |
| (18) Prostate tissue | Basal prostatic cells |
| | Prostatic glandular cells |
| | Urothelial cells |
| | Endothelial cells |
| | Fibroblasts |
| | Smooth muscle cells |
| | Macrophages |
| | T-cells; |
| (19) Rectum tissue | Undifferentiated cells |
| | Intestinal goblet cells |
| | Paneth cells |
| | Distal enterocytes |
| | Enteroednocrine cells; |
| (20) Skin tissue | Langerhans cells |
| | Fibroblasts |
| | Endothelial cells |
| | Basal keratinocytes |
| | Suprabasal keratinocytes |
| | T-cells |
| | Smooth muscle cells |
| | Melanocytes; |
| (21) PBMC tissue | Monocytes |
| | T-cells |
| | NK-cells |
| | Dendritic cells; |
| (22) Small intestine tissue | Proximal enterocytes |
| | Undifferentiated cells |
| | Intestinal goblet cells |
| | Paneth cells; |
| (23) Spleen tissue | B-cells |
| | T-cells |
| | Plasma cells |
| | Macrophages; |
| (24) Stomach tissue | B-cells |
| | T-cells |
| | Gastric mucus-secreting |
| | Plasma cells |
| | Fibroblasts |
| | Macrophages; |
| (25) Testes tissue | Leydig cells |
| | Late spermatids |
| | Spermatogonia |
| | Early spermatids |
| | Macrophages |
| | Spermatocytes |
| | Peritubular cells |
| | Sertoli cells |
| | Endothelial cells; |
| (26) Peripheral nervous system | Motor neurons |
| tissue | Sensory neurons |
| | Schwann cells |
| | Dorsal root ganglion; |
| (27) Bone/cartilage/joint tissue | Chondrocytes |
| | Chondroblasts |
| | Mesenchymal cells |
| | Osteoblasts |
| | Osteoclasts. |

104. The method of any one of claims 84-103 wherein the method comprises piercing the body of the subject with a needle of a syringe and injecting the antigen-binding protein that binds specifically to transferrin receptor or an antigenic-fragment thereof or variant thereof to the subject conjugated to the molecular cargo into the body of the subject.

105. The method of any one of claims 84-104 wherein the subject suffers from a muscle atrophy condition, metabolic disease, sarcopenia or cachexia.


SEQUENCE LISTING (575 sequences)

SEQ ID NO: 1 - DNA, 363 bp, synthetic construct
  source 1..363: mol_type = other DNA
gaggtgcagctggtggagtctgggggaggcttggtacagcctggggggtccctgagactctcctgtgcagcctctggattcgcctttagcagctatgccatgacctgggtccgacaggctccagggaaggggctggagtgggtctcagttatcagtggtactggtggtagtacatactacgcagactccgtgaagggccggttcaccatctccagagacaattccaagaacacgctgtatctacaaatgaacagcctgagagccgaggacacggccgtatattactgtgcgaaagggggagcagctcgtagaatggaatacttccagtactggggccagggcaccctggtcaccgtctcctca

SEQ ID NO: 2 - protein, 121 aa, synthetic construct
EVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTLVTVSS

SEQ ID NO: 3 - protein, 8 aa, synthetic construct
GFAFSSYA

SEQ ID NO: 4 - protein, 8 aa, synthetic construct
ISGTGGST

SEQ ID NO: 5 - protein, 14 aa, synthetic construct
AKGGAARRMEYFQY

SEQ ID NO: 6 - DNA, 321 bp, synthetic construct
  source 1..321: mol_type = other DNA
gacatccagatgacccagtctccatcctccctgtctgcatctgtaggagacagagtcaccatcacttgccgggcgagtcagggcattagcaattatttagcctggtatcagcagaaaccagggaaagttcctaacctccttatctatgctgcatccactttgcaatcaggggtcccatctcgattcagtggcagtggatctgggacagatttcactctcaccatcagcagcctgcagcctgaagatgttgcaacttattactgtcaaaagtataacagtgcccctctcactttcggcggagggaccaaggtggagatcaaa

SEQ ID NO: 7 - protein, 107 aa, synthetic construct
DIQMTQSPSSLSASVGDRVTITCRASQGISNYLAWYQQKPGKVPNLLIYAASTLQSGVPSRFSGSGSGTDFTLTISSLQPEDVATYYCQKYNSAPLTFGGGTKVEIK

SEQ ID NO: 8 - protein, 6 aa, synthetic construct
QGISNY

SEQ ID NO: 9 - skipped

SEQ ID NO: 10 - protein, 9 aa, synthetic construct
QKYNSAPLT

SEQ ID NO: 11 - DNA, 363 bp, synthetic construct
  source 1..363: mol_type = other DNA
gaggtgcagctggtggagtctgggggaggcttggtacagcctggggggtccctgagactctcctgtgcagcctctggattcacctttaacagctatgccatgacctgggtccgccaggctccagggaaggggctggagtgggtctcatttattggtggtagtactggtaacacatactacgcaggctccgtgaagggccggttcaccatctccagcgacaattccaagaagacgctgtatctgcaaatgaacagcctgagagccgaggacacggccgtatattactgtgcgaaagggggagcagctcgtagaatggaatacttccagcactggggccagggcaccctggtcaccgtctcctca

SEQ ID NO: 12 - protein, 121 aa, synthetic construct
EVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTLVTVSS

SEQ ID NO: 13 - protein, 8 aa, synthetic construct
GFTFNSYA

SEQ ID NO: 14 - protein, 8 aa, synthetic construct
IGGSTGNT

SEQ ID NO: 15 - protein, 14 aa, synthetic construct
AKGGAARRMEYFQH

SEQ ID NO: 16 - DNA, 321 bp, synthetic construct
  source 1..321: mol_type = other DNA
gacatccagatgacccagtctccatcctccctgtctgcatctataggagacagagtcaccatcacttgccgggcgagtcagggcattagcaattatttagcctggtatcaacagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaaccataacagtgtccctctcactttcggcggagggaccaaggtggagatcaaa

SEQ ID NO: 17 - protein, 107 aa, synthetic construct
DIQMTQSPSSLSASIGDRVTITCRASQGISNYLAWYQQKPGKVPKLLIYAASTLQSGVPSRFSGSGSGTDFTLTISSLQPEDVATYYCQNHNSVPLTFGGGTKVEIK

SEQ ID NO: 18 - protein, 6 aa, synthetic construct
QGISNY

SEQ ID NO: 19 - skipped

SEQ ID NO: 20 - protein, 9 aa, synthetic construct
QNHNSVPLT

SEQ ID NO: 21 - DNA, 366 bp, synthetic construct
  source 1..366: mol_type = other DNA
caggtgcagctggtggagtctgggggaggcgtggtccagcctgggaggtccctgagactctcctgtgcagcgtctggattcaccttcactacctatggcatgcactgggtccgccaggctccaggcaaggggctggagtgggtggctgttatatggtatgatggaagtaataaatattatggagactccgtgaagggccgattcaccatctccagagacaattccaagaacacactgtatctgcaaatgaacagcctgagagtcgacgacacggctgtttattactgtacgagaacccatggctataccaggtcgtcggacggttttgactactggggccagggaaccctggtcaccgtctcctca

SEQ ID NO: 22 - protein, 122 aa, synthetic construct
QVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTLVTVSS

SEQ ID NO: 23 - protein, 8 aa, synthetic construct
GFTFTTYG

SEQ ID NO: 24 - protein, 8 aa, synthetic construct
IWYDGSNK

SEQ ID NO: 25 - protein, 15 aa, synthetic construct
TRTHGYTRSSDGFDY

SEQ ID NO: 26 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagaaatgttttaggctggtttcagcagaaaccagggaaagcccctcagcgcctgatctatgctgcatccagtttgcaaagtggggtcccatcaaggttcagcggcagtggatctgggacagaattcactctcacaatcagcagcctacagcctgaagattttgcaacttattactgtctacagcataatttttacccgctcactttcggcggagggaccaaggtggagatcaaa

SEQ ID NO: 27 - protein, 107 aa, synthetic construct
DIQMTQSPSSLSASVGDRVTITCRASQSIRNVLGWFQQKPGKAPQRLIYAASSLQSGVPSRFSGSGSGTEFTLTISSLQPEDFATYYCLQHNFYPLTFGGGTKVEIK

SEQ ID NO: 28 - protein, 6 aa, synthetic construct
QSIRNV

SEQ ID NO: 29 - skipped

SEQ ID NO: 30 - protein, 9 aa, synthetic construct
LQHNFYPLT

SEQ ID NO: 31 - DNA, 369 bp, synthetic construct
  source 1..369: mol_type = other DNA
gaagtgcagctggtggagtctgggggaggcttggtacagcctggcaggtccctgagactctcctgtgcagcctctggattcacctttgatgataaagccatgcactgggtccggcaagttccagggaagggcctggaatggatctcaggtattagttggaatagtggtactataggctatgcggactctgtgaagggccgattcatcatctccagagacaacgccaagaactccctgtatctacaaatgaacagtctgagagctgaggacacggccttgtattactgcgcaaaagatggagataccagtggctggtactggtacggtttggacgtctggggccaagggaccacggtcaccgtctcctca

SEQ ID NO: 32 - protein, 123 aa, synthetic construct
EVQLVESGGGLVQPGRSLRLSCAASGFTFDDKAMHWVRQVPGKGLEWISGISWNSGTIGYADSVKGRFIISRDNAKNSLYLQMNSLRAEDTALYYCAKDGDTSGWYWYGLDVWGQGTTVTVSS

SEQ ID NO: 33 - protein, 8 aa, synthetic construct
GFTFDDKA

SEQ ID NO: 34 - protein, 8 aa, synthetic construct
ISWNSGTI

SEQ ID NO: 35 - protein, 16 aa, synthetic construct
AKDGDTSGWYWYGLDV

SEQ ID NO: 36 - DNA, 321 bp, synthetic construct
  source 1..321: mol_type = other DNA
gaaattgtgttgacacagtctcctgccaccctgtctttgtctccaggggaaagagccaccctctcctgcagggccagtcagagtgttagcagctacttagcctggtaccaacagaaacctggccaggctcccaggctcctcatccatgatgtatccaacagggccactggcatcccagccaggttcagtggcagtgggtctgggacagacttcactctcaccatcagcagtctagagcctgaagattttgtagtttattactgtcagcagcgtagcgactggcccatcaccttcggccaagggacacgactggagattaaa

SEQ ID NO: 37 - protein, 107 aa, synthetic construct
EIVLTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIK

SEQ ID NO: 38 - protein, 6 aa, synthetic construct
QSVSSY

SEQ ID NO: 39 - skipped

SEQ ID NO: 40 - protein, 9 aa, synthetic construct
QQRSDWPIT

SEQ ID NO: 41 - DNA, 360 bp, synthetic construct
  source 1..360: mol_type = other DNA
caggtgcagctggtggagtctgggggaggcgtggtccagcctgggaggtccctgagactctcctgtatagcctctggattcaccttcagtgtctatggcattcactgggtccgccaggctccaggcaaggggctggagtggatggcagtaatatcacatgatggaaatattaaacactatgcagactccgtgaagggccgattcaccatctccagagacaattccaagaacacgctgtatcttcaaattaacagcctgagaactgaggacacggctgtgtattactgtgcgaaagatacctggaactcccttgatacttttgatatctggggccaagggacaatggtcaccgtctcttca

SEQ ID NO: 42 - protein, 120 aa, synthetic construct
QVQLVESGGGVVQPGRSLRLSCIASGFTFSVYGIHWVRQAPGKGLEWMAVISHDGNIKHYADSVKGRFTISRDNSKNTLYLQINSLRTEDTAVYYCAKDTWNSLDTFDIWGQGTMVTVSS

SEQ ID NO: 43 - protein, 8 aa, synthetic construct
GFTFSVYG

SEQ ID NO: 44 - protein, 8 aa, synthetic construct
ISHDGNIK

SEQ ID NO: 45 - protein, 13 aa, synthetic construct
AKDTWNSLDTFDI

SEQ ID NO: 46 - DNA, 321 bp, synthetic construct
  source 1..321: mol_type = other DNA
gacatccagttgacccagtctccatcctccctgtctgcatctgtaggagacagagtcaccatcacttgctgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccctctcactttcggcggagggaccaaggtggagatcaaa

SEQ ID NO: 47 - protein, 107 aa, synthetic construct
DIQLTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 48 - protein, 6 aa, synthetic construct
QGISSY

SEQ ID NO: 49 - skipped

SEQ ID NO: 50 - protein, 9 aa, synthetic construct
QQLNSYPLT

SEQ ID NO: 51 - DNA, 360 bp, synthetic construct
  source 1..360: mol_type = other DNA
caggtcaccttgagggagtctggtcccgcgctggtgaaaccctcacagaccctcacactgacctgcaccttctctggattctcactcaacacttatgggatgtttgtgagctggatccgtcagcctccagggaaggccctagagtggcttgcacacattcattgggatgatgataaatactacagcacatctctgaagaccaggctcaccatctccaaggacacctccaaaaaccaggtggtccttacaatgaccaacatggaccctgtggacacagccacgtattattgtgcacgggggcacaataatttgaactacatcatccactggggccagggaaccctggtcaccgtctcctca

SEQ ID NO: 52 - protein, 120 aa, synthetic construct
QVTLRESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSS

SEQ ID NO: 53 - protein, 10 aa, synthetic construct
GFSLNTYGMF

SEQ ID NO: 54 - protein, 7 aa, synthetic construct
IHWDDDK

SEQ ID NO: 55 - protein, 12 aa, synthetic construct
ARGHNNLNYIIH

SEQ ID NO: 56 - DNA, 321 bp, synthetic construct
  source 1..321: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaagctcctgatctatgctgcatccactttacaaagtggggtcccatcaaggttcagcggcagtggatctggcacagatttcactctcaccatcagcagcctgcagcctgaagattttgcaacttattactgtctacaagattacaattacccattcactttcggccctgggaccaaagtggatatcaaa

SEQ ID NO: 57 - protein, 107 aa, synthetic construct
AIQMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVDIK

SEQ ID NO: 58 - protein, 6 aa, synthetic construct
QGIRND

SEQ ID NO: 59 - skipped

SEQ ID NO: 60 - protein, 9 aa, synthetic construct
LQDYNYPFT

SEQ ID NO: 61 - DNA, 348 bp, synthetic construct
  source 1..348: mol_type = other DNA
gaggtgcagctggtggagtctgggggaggcttggtacagcctggagggtccctgagactctcctgtgcagtctctggattcatcttcagtagttatgaaatgaactgggtccgccaggctccagggaaggggctggagtgggtttcatacattagtagtagtggtagtaccatattctacgcagactctgtgaagggccgattcaccatctccagagacaacgccaagaactcactgtatctgcaaatgaacagcctgagagccgaggacacggctgtttattactgtgtgtctggagtggtcctttttgatgtctggggccaagggacaatggtcaccgtctcttca

SEQ ID NO: 62 - protein, 116 aa, synthetic construct
EVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSS

SEQ ID NO: 63 - protein, 8 aa, synthetic construct
GFIFSSYE

SEQ ID NO: 64 - protein, 8 aa, synthetic construct
ISSSGSTI

SEQ ID NO: 65 - protein, 9 aa, synthetic construct
VSGVVLFDV

SEQ ID NO: 66 - DNA, 321 bp, synthetic construct
  source 1..321: mol_type = other DNA
gaaatagtgatgacgcagtctccagccaccctgtctgtgtctccgggggaaagagccaccctctcctgcagggccagtcagagtgttagcagcaactttgcctggtaccaacagaaacctggccaggctcccaggctcctcatctatagtgcatcctccagggccactggtatcccagtcaggttcagtggcagtgggtctgggacagagttcactctcaccatcagcagcctgcagtctgaagattttgcagtttattactgtcagcagtataatatctggcctcggacgttcggccaagggaccaaggtggaaatcaaa

SEQ ID NO: 67 - protein, 107 aa, synthetic construct
EIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIK

SEQ ID NO: 68 - protein, 6 aa, synthetic construct
QSVSSN

SEQ ID NO: 69 - skipped

SEQ ID NO: 70 - protein, 9 aa, synthetic construct
QQYNIWPRT

SEQ ID NO: 71 - DNA, 375 bp, synthetic construct
  source 1..375: mol_type = other DNA
gaggtgcagctggtggagtctgggggaggcttggtccagcctggggggtccctgagactctcctgtgcagcctctggattcacctttagtaactattggatgacctgggtccgccaggctccagggaaggggctggagtgggtggccaacataaaggaagatggaagtgagaaagactatgtggactctgtgaagggccgattcaccatctccagagacaacgccaagaactcactgtatctgcaaatgaacagcctgagaggcgaggacacggctgtgtattactgtgcgagagatggggagcagctcgtcgattactactactactacgttatggacgtctggggccaagggaccacggtcaccgtctcctca

SEQ ID NO: 72 - protein, 125 aa, synthetic construct
EVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSS

SEQ ID NO: 73 - protein, 8 aa, synthetic construct
GFTFSNYW

SEQ ID NO: 74 - protein, 8 aa, synthetic construct
IKEDGSEK

SEQ ID NO: 75 - protein, 18 aa, synthetic construct
ARDGEQLVDYYYYYVMDV

SEQ ID NO: 76 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaaaaggctaacagtttcccgtacacttttggccaggggaccaagctggagatcaaa

SEQ ID NO: 77 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKLEIK

SEQ ID NO: 78 - protein, 6 aa, synthetic construct
QGISSW

SEQ ID NO: 79 - skipped

SEQ ID NO: 80 - protein, 9 aa, synthetic construct
QKANSFPYT

SEQ ID NO: 81 - DNA, 381 bp, synthetic construct
  source 1..381: mol_type = other DNA
gaagtgcagctggtggagtctgggggaggcttggtacagcctggcaggtccctgagactctcctgtgcagcctctggattcacctttgatgactatgccatgcactgggtccggcaagctccagggaagggcctggagtgggtctcaggtattagttggaatagtggttacataggctatgcggactctgtgaagggccgattcaccatctccagagacaacgccgagaactccctacatctgcaaatgaacagtctgagagctgaggacacggccttgtattactgtgcaagagggggatctactctggttcggggagttaagggaggctactacggtatggacgtctggggccaagggaccacggtcaccgtctcctca

SEQ ID NO: 82 - protein, 127 aa, synthetic construct
EVQLVESGGGLVQPGRSLRLSCAASGFTFDDYAMHWVRQAPGKGLEWVSGISWNSGYIGYADSVKGRFTISRDNAENSLHLQMNSLRAEDTALYYCARGGSTLVRGVKGGYYGMDVWGQGTTVTVSS

SEQ ID NO: 83 - protein, 8 aa, synthetic construct
GFTFDDYA

SEQ ID NO: 84 - protein, 8 aa, synthetic construct
ISWNSGYI

SEQ ID NO: 85 - protein, 20 aa, synthetic construct
ARGGSTLVRGVKGGYYGMDV

SEQ ID NO: 86 - DNA, 321 bp, synthetic construct
  source 1..321: mol_type = other DNA
gacatccagatgacccagtctccatcctccctgtctgcatctgtaggagacagagtcaccatcacttgccgggcaagtcagagcataagtagctatttaaattggtatcagcagaaaccaggtaaagcccctaaggtcctgatctatgctgcatccagtttgcaaagtggggtcccatcaaggttcagtggcagtggatctgggacagatttcactctcaccatcagcagtctgcaacctgaagattttgcaacttactactgtcaacagagttacagtattccgctcactttcggcggagggaccaaggtggagatcaaa

SEQ ID NO: 87 - protein, 107 aa, synthetic construct
DIQMTQSPSSLSASVGDRVTITCRASQSISSYLNWYQQKPGKAPKVLIYAASSLQSGVPSRFSGSGSGTDFTLTISSLQPEDFATYYCQQSYSIPLTFGGGTKVEIK

SEQ ID NO: 88 - protein, 6 aa, synthetic construct
QSISSY

SEQ ID NO: 89 - skipped

SEQ ID NO: 90 - protein, 9 aa, synthetic construct
QQSYSIPLT

SEQ ID NO: 91 - DNA, 351 bp, synthetic construct
  source 1..351: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacatttccaagaacacgctgtatctgcaaatgaacagcctgagagccgaggacacggccgtatattactgtgcgggtcaactggatctcttctttgactactggggccagggaaccctggtcaccgtctcctca

SEQ ID NO: 92 - protein, 117 aa, synthetic construct
QVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSS

SEQ ID NO: 93 - protein, 8 aa, synthetic construct
GFTFSSYG

SEQ ID NO: 94 - protein, 8 aa, synthetic construct
IWYDGSNK

SEQ ID NO: 95 - protein, 10 aa, synthetic construct
AGQLDLFFDY

SEQ ID NO: 96 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattgacaggtatttaaattggtatcggcagaaaccagggaaagcccctaagctcctgatctatactacatccagtttgcaaagtggggtcccatcaaggttcagtggcagtggatctgggacagatttcactctcaccctcagcagtctgcagcctgaagattttgcaacttactactgtcagcagagttacagtcccccgctcactttcggcggagggaccaaggtggagatcaaa

SEQ ID NO: 97 - protein, 107 aa, synthetic construct
DIQMTQSPSSLSASVGDRVTITCRASQSIDRYLNWYRQKPGKAPKLLIYTTSSLQSGVPSRFSGSGSGTDFTLTLSSLQPEDFATYYCQQSYSPPLTFGGGTKVEIK

SEQ ID NO: 98 - protein, 6 aa, synthetic construct
QSIDRY

SEQ ID NO: 99 - skipped

SEQ ID NO: 100 - protein, 9 aa, synthetic construct
QQSYSPPLT

SEQ ID NO: 101 - DNA, 375 bp, synthetic construct
  source 1..375: mol_type = other DNA
gaggtgcagctggtggagtctgggggaggcttggtccagcctggggggtccctgagactctcctgtacagcctctggattcacctttagtaactattggatgacctgggtccgccaggctccagggaaggggctggagtgggtggccaacataaaggaagatggaagtgagaaagagtatgtggactctgtgaagggccggttcaccatctccagagacaacgccaagaattcactgtatctgcaaatgaacagcctgagaggcgaggacacggctgtatattactgtgcgagagatggggagcagctcgtcgattactattactactacgttatggacgtctggggccaagggaccacggtcaccgtctcctca

SEQ ID NO: 102 - protein, 125 aa, synthetic construct
EVQLVESGGGLVQPGGSLRLSCTASGFTFSNYWMTWVRQAPGKGLEWVANIKEDGSEKEYVDSVKGRFTISRDNAKNSLYLQMNSLRGEDTAVYYCARDGEQLVDYYYYYVMDVWGQGTTVTVSS

SEQ ID NO: 103 - protein, 8 aa, synthetic construct
GFTFSNYW

SEQ ID NO: 104 - protein, 8 aa, synthetic construct
IKEDGSEK

SEQ ID NO: 105 - protein, 18 aa, synthetic construct
ARDGEQLVDYYYYYVMDV

SEQ ID NO: 106 - DNA, 321 bp, synthetic construct
  source 1..321: mol_type = other DNA
gacatccagatgacccagtctccatcttccgtgtctgcatctgttggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaaaaggctgacagtctcccgtacgcttttggccaggggaccaagctggagatcaaa

SEQ ID NO: 107 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKADSLPYAFGQGTKLEIK

SEQ ID NO: 108 - protein, 6 aa, synthetic construct
QGISSW

SEQ ID NO: 109 - skipped

SEQ ID NO: 110 - protein, 9 aa, synthetic construct
QKADSLPYA

SEQ ID NO: 111 - DNA, 363 bp, synthetic construct
  source 1..363: mol_type = other DNA
gaggtgcagctggtggagtctgggggaggcttggttcagcctggggggtccctgagactctcctgtgcaacctctggattcacctttaccagctatgacatgaagtgggtccgccaggctccagggctgggcctggagtgggtctcagctattagtggtagtggtggtaacacatactacgcagactccgtgaagggccggttcaccatctccagagacaattccaggaacacgctgtatctgcaaatgaacagcctgagagccgaggacacggccgtatattactgtacgaggtcccatgacttcggtgccttcgactactttgactactggggccagggaaccctggtcaccgtctcctca

SEQ ID NO: 112 - protein, 121 aa, synthetic construct
EVQLVESGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTLVTVSS

SEQ ID NO: 113 - protein, 8 aa, synthetic construct
GFTFTSYD

SEQ ID NO: 114 - protein, 8 aa, synthetic construct
ISGSGGNT

SEQ ID NO: 115 - protein, 14 aa, synthetic construct
TRSHDFGAFDYFDY

SEQ ID NO: 116 - DNA, 321 bp, synthetic construct
  source 1..321: mol_type = other DNA
gacatccagatgacccagtctccatcctccctgtctgcatctgtgggagacagagtcaccatcacttgccgggcaagtcagggcattagagatcattttggctggtatcagcagaaaccagggaaagcccctaagcgcctgatctatgctgcatccagtttgcacagtggggtcccatcaaggttcagcggcagtggatctgggacagaattcactctcacaatcagcagcttgcagcctgaagattttgcaacctattactgtctacagtatgatacttacccgctcactttcggcggagggaccaaggtggagatcaaa

SEQ ID NO: 117 - protein, 107 aa, synthetic construct
DIQMTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIK

SEQ ID NO: 118 - protein, 6 aa, synthetic construct
QGIRDH

SEQ ID NO: 119 - skipped

SEQ ID NO: 120 - protein, 9 aa, synthetic construct
LQYDTYPLT

SEQ ID NO: 121 - DNA, 366 bp, synthetic construct
  source 1..366: mol_type = other DNA
gaagtgcagctggtggagtctgggggagacttggtacagcctggcaggtccctgagactctcctgtgcagcctctggattcacctttgatgattatgccatgcactgggtccggcaagctccagggaagggcctggagtgggtctcaggtattagttggaatagtgctaccagagtctatgcggactctgtgaagggccgattcaccatctccagagacaacgccaagaatttcctgtatctgcaaatgaacagtctgagatctgaggacacggccttgtatcactgtgcaaaagatatggatatctcgctagggtactacggtttggacgtctggggccaagggaccacggtcaccgtctcctca

SEQ ID NO: 122 - protein, 122 aa, synthetic construct
EVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSS

SEQ ID NO: 123 - protein, 8 aa, synthetic construct
GFTFDDYA

SEQ ID NO: 124 - protein, 8 aa, synthetic construct
ISWNSATR

SEQ ID NO: 125 - protein, 15 aa, synthetic construct
AKDMDISLGYYGLDV

SEQ ID NO: 126 - DNA, 324 bp, synthetic construct
  source 1..324: mol_type = other DNA
gaaatagtgatgacgcagtctccagccaccctgtctgtgtctccaggggaaagagccaccctctcctgcagggccagtcagactgttagcagcaacttagcctggtatcagcagaaacctggccaggctcccaggctcctcatctatggttcatcctccagggccactggtatcccagccaggttcagtggcagtgggtctgggacagagttcactctcaccatcagcagcctgcagtctgaagattttgcagtttattactgtcagcagtataataactggcctccctacacttttggccaggggaccaagctggagatcaaa

SEQ ID NO: 127 - protein, 108 aa, synthetic construct
EIVMTQSPATLSVSPGERATLSCRASQTVSSNLAWYQQKPGQAPRLLIYGSSSRATGIPARFSGSGSGTEFTLTISSLQSEDFAVYYCQQYNNWPPYTFGQGTKLEIK

SEQ ID NO: 128 - protein, 6 aa, synthetic construct
QTVSSN

SEQ ID NO: 129 - skipped

SEQ ID NO: 130 - protein, 10 aa, synthetic construct
QQYNNWPPYT

SEQ ID NO: 131 - DNA, 372 bp, synthetic construct
  source 1..372: mol_type = other DNA
cagatcaccttgaaggagtctggtcctacgctggtgaaacccacacagaccctcacgctgacctgcaccttctctgggttctcactcagcactagtggagtgggtgtggtctggatccgtcagccccccggaaaggccctggagtggcttgcactcatttattggaatgatcataagcggtacagcccatctctggggagcaggctcaccatcaccaaggacacctccaaaaaccaggtggtccttacaatgaccaacatggaccctgtggacacagccacatattactgtgcacactacagtgggagctattcctactactactatggtttggacgtctggggccaagggaccacggtcaccgtctcctca

SEQ ID NO: 132 - protein, 124 aa, synthetic construct
QITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSS

SEQ ID NO: 133 - protein, 10 aa, synthetic construct
GFSLSTSGVG

SEQ ID NO: 134 - protein, 7 aa, synthetic construct
IYWNDHK

SEQ ID NO: 135 - protein, 16 aa, synthetic construct
AHYSGSYSYYYYGLDV

SEQ ID NO: 136 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattgccagctggttagcctggtatcagcagaaaccagggaaagcccctgagctcctgatctatgctgcatccagtttgcaaggtggggtcccatcaaggttcagcggcagtggatctgggacagatttcactctcaccatcagcagcctgcagcctgaagattttgcaatttactattgtcaacaggctaactatttcccgtggacgttcggccaagggaccaaggtggaaatcaaa

SEQ ID NO: 137 - protein, 107 aa, synthetic construct
DIQMTQSPSSVSASVGDRVTITCRASQGIASWLAWYQQKPGKAPELLIYAASSLQGGVPSRFSGSGSGTDFTLTISSLQPEDFAIYYCQQANYFPWTFGQGTKVEIK

SEQ ID NO: 138 - protein, 6 aa, synthetic construct
QGIASW

SEQ ID NO: 139 - skipped

SEQ ID NO: 140 - protein, 9 aa, synthetic construct
QQANYFPWT

SEQ ID NO: 141 - DNA, 363 bp, synthetic construct
  source 1..363: mol_type = other DNA
gaggtgcagctgttggagtctgggggagccttggtacagcctggggggtccctgagactctcctgtgcagcctctggattcacctttacctcctatgccatgcactgggtccgccaggctccagggaagggtctggagtgggtctcatctattagaggtagtggtggtggcacatactccgcagactccgtgaagggccggttcaccatctccagagacaattccagggacactctatatctgcaaatgaacagtgtgagagccgaggacacggccgtttattactgtgcgaggtcccatgactacggtgccttcgacttctttgactactggggccagggaaccctggtcaccgtctcctca

SEQ ID NO: 142 - protein, 121 aa, synthetic construct
EVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTLVTVSS

SEQ ID NO: 143 - protein, 8 aa, synthetic construct
GFTFTSYA

SEQ ID NO: 144 - protein, 8 aa, synthetic construct
IRGSGGGT

SEQ ID NO: 145 - protein, 14 aa, synthetic construct
ARSHDYGAFDFFDY

SEQ ID NO: 146 - DNA, 321 bp, synthetic construct
  source 1..321: mol_type = other DNA
gacatccagatgacccagtctccatcctccctgtctgcatctgtaggagacagagtcaccatcacttgccgggcaagtcagggcattagaactgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcggcctgaagattttgcaactttttactgtctacagtataatagttacccgctcactttcggcggagggaccaaggtggagatcaaa

SEQ ID NO: 147 - protein, 107 aa, synthetic construct
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVEIK

SEQ ID NO: 148 - protein, 6 aa, synthetic construct
QGIRTD

SEQ ID NO: 149 - skipped

SEQ ID NO: 150 - protein, 9 aa, synthetic construct
LQYNSYPLT

SEQ ID NO: 151 - DNA, 348 bp, synthetic construct
  source 1..348: mol_type = other DNA
caggtgcagctggtggagtctgggggaggcttggtcaagcctggagggtccctgagactctcctgtgcagcctctggattcaccttcagtgactacttcatgagctggatccgccaggctccagggaaggggctggagtgggtttcatacattagtagtactggtagtaccataaattatgcagactctgtgaagggccgattcaccatctccagggacaatgtcaagaattcactgtatctgcaaatgaccagcctgagagtcgaggacacggccgtgtattactgtacgagagataactggaactatgaatactggggccagggaaccctggtcaccgtctcctca

SEQ ID NO: 152 - protein, 116 aa, synthetic construct
QVQLVESGGGLVKPGGSLRLSCAASGFTFSDYFMSWIRQAPGKGLEWVSYISSTGSTINYADSVKGRFTISRDNVKNSLYLQMTSLRVEDTAVYYCTRDNWNYEYWGQGTLVTVSS

SEQ ID NO: 153 - protein, 8 aa, synthetic construct
GFTFSDYF

SEQ ID NO: 154 - protein, 8 aa, synthetic construct
ISSTGSTI

SEQ ID NO: 155 - protein, 9 aa, synthetic construct
TRDNWNYEY

SEQ ID NO: 156 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcatcaacttagcctggtaccagcagaaacctggccaggctcccaggctcctcatctttgttgcatccaccagggccactggtatcccagccaggttcagtggcagtgggtctgggacagagttcactctcaccatcagcagcctgcagtctgaagattttgcaacttattactgtcagcagtatgatatctggccgtacacttttggccaggggaccaagctggagatcaaa

SEQ ID NO: 157 - protein, 107 aa, synthetic construct
EIVMTQSPATLSVSPGERATLSCRASQSVSINLAWYQQKPGQAPRLLIFVASTRATGIPARFSGSGSGTEFTLTISSLQSEDFATYYCQQYDIWPYTFGQGTKLEIK

SEQ ID NO: 158 - protein, 6 aa, synthetic construct
QSVSIN

SEQ ID NO: 159 - skipped

SEQ ID NO: 160 - protein, 9 aa, synthetic construct
QQYDIWPYT

SEQ ID NO: 161 - DNA, 369 bp, synthetic construct
  source 1..369: mol_type = other DNA
cagctgcagctgcaggagtcgggcccaggactggtgaagccttcggagaccctgtccctcacctgcactgtgtctggtgaatccatcagcagtaatacttactactggggctggatccgccagcccccagggaaggggctggaatggattgggagtatcgattatagtgggaccaccaattataacccgtccctcaagagtcgagtcaccatatccgtagacacgtccaggaatcacttctccctgaggctgaggtctgtgaccgccgcagacacggctgtgtattactgtgcgagagagtggggaaactacggctactattacggtatggacgtttggggccaagggaccacggtcaccgtctcctca

SEQ ID NO: 162 - protein, 123 aa, synthetic construct
QLQLQESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSS

SEQ ID NO: 163 - protein, 10 aa, synthetic construct
GESISSNTYY

SEQ ID NO: 164 - protein, 7 aa, synthetic construct
IDYSGTT

SEQ ID NO: 165 - protein, 15 aa, synthetic construct
AREWGNYGYYYGMDV

SEQ ID NO: 166 - DNA, 321 bp, synthetic construct
  source 1..321: mol_type = other DNA
gacatccagatgacccagtctccatcctccctgtctgcatctgtaggagacagagtcaccatcaattgccgggcaagtcagggcattagaaatgatttaggctggtatcagcagaaaccagggaaagcccctaagcgcctgatctatgctgcatccagtttgcaaagtggggtcccattaaggttcagtggcagtggatctgggacagaattcactctcacaatcaacaacctgcagcctgaagattttgcaacttattactgtctatcgcataatagttacccgtggacgttcggccaagggaccaaggtggaaatcaaa

SEQ ID NO: 167 - protein, 107 aa, synthetic construct
DIQMTQSPSSLSASVGDRVTINCRASQGIRNDLGWYQQKPGKAPKRLIYAASSLQSGVPLRFSGSGSGTEFTLTINNLQPEDFATYYCLSHNSYPWTFGQGTKVEIK

SEQ ID NO: 168 - protein, 6 aa, synthetic construct
QGIRND

SEQ ID NO: 169 - skipped

SEQ ID NO: 170 - protein, 9 aa, synthetic construct
LSHNSYPWT

SEQ ID NO: 171 - DNA, 354 bp, synthetic construct
  source 1..354: mol_type = other DNA
caggtgcagctggtgcagtctggggctgaggtgaagaagcctgggtcctcggtgagggtctcctgcaaggcttctagaggcaccttcagcagctatgctatcagctgggtgcgacaggcccctggacaaggccttgagtggatgggagggatcatccccatctttggtacagcaaactacgcacagaagttcctggccagagtcacgattaccgcggacgaatccacgagcacagcctacatggagctgagcagcctgagatctgaggacacggccgtgtattactgtgcgagagagaaggggtggaactactttgactactggggccagggaaccctggtcaccgtctcctca

SEQ ID NO: 172 - protein, 118 aa, synthetic construct
QVQLVQSGAEVKKPGSSVRVSCKASRGTFSSYAISWVRQAPGQGLEWMGGIIPIFGTANYAQKFLARVTITADESTSTAYMELSSLRSEDTAVYYCAREKGWNYFDYWGQGTLVTVSS

SEQ ID NO: 173 - protein, 8 aa, synthetic construct
RGTFSSYA

SEQ ID NO: 174 - protein, 8 aa, synthetic construct
IIPIFGTA

SEQ ID NO: 175 - protein, 11 aa, synthetic construct
AREKGWNYFDY

SEQ ID NO: 176 - DNA, 321 bp, synthetic construct
  source 1..321: mol_type = other DNA
gacatccagatgacccagtctccaccttccgtgtctgcatctgtaggagacagagtcaccatcacttgtcgggcgagtcagggtattagcagctggttagcctggtatcagcagaaaccagggaaagcccctaaactcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccctcggacgttcggccaagggaccaaggtggaaatcaaa

SEQ ID NO: 177 - protein, 107 aa, synthetic construct
DIQMTQSPPSVSASVGDRVTITCRASQGISSWLAWYQQKPGKAPKLLIYAASSLQSGVPSRFSGSGSGTDFTLTISSLQPEDFATYYCQQANSFPRTFGQGTKVEIK

SEQ ID NO: 178 - protein, 6 aa, synthetic construct
QGISSW

SEQ ID NO: 179 - skipped

SEQ ID NO: 180 - protein, 9 aa, synthetic construct
QQANSFPRT

SEQ ID NO: 181 - DNA, 369 bp, synthetic construct
  source 1..369: mol_type = other DNA
caggtgcagctggtggagtctgggggaggcttggtcaagcctggagggtccctgagactctcctgtgcagcctctggattcaccttcagtgactactacatgaactggatccgccaggctccagggaaggggctggagtgggtttcatacattagtagtagtgggactaccatatactacgcagactctgtgaagggccgattcaccatctccagggacaacgccaagaaatcactgtatctggagatgaacagcctcagagccgaggacacggccgtgtactactgtgcgagagaggggtacggtaatgactactattactacggtatagacgtctggggccaagggaccacggtcaccgtctcctca

SEQ ID NO: 182 - protein, 123 aa, synthetic construct
QVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSS

SEQ ID NO: 183 - protein, 8 aa, synthetic construct
GFTFSDYY

SEQ ID NO: 184 - protein, 8 aa, synthetic construct
ISSSGTTI

SEQ ID NO: 185 - protein, 16 aa, synthetic construct
AREGYGNDYYYYGIDV

SEQ ID NO: 186 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatggtaatggatacaactatttgacttggtacctgcagaagccagggcagtctccacagctcctgatctatttgggttctaatcgggcctccggggtccctgacaggttcagtggcagtggatcaggcacagattttacactgaaaataagcagagtggaggctgaggatgttggggtttattactgcatgcaagctctacaaactccgtacacttttggccaggggaccaagctggagatcaaa

SEQ ID NO: 187 - protein, 112 aa, synthetic construct
DIVMTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKLEIK

SEQ ID NO: 188 - protein, 11 aa, synthetic construct
QSLLHGNGYNY

SEQ ID NO: 189 - skipped

SEQ ID NO: 190 - protein, 9 aa, synthetic construct
MQALQTPYT

SEQ ID NO: 191 - DNA, 366 bp, synthetic construct
  source 1..366: mol_type = other DNA
caggtgcagctggtggagtctgggggaggcgtggtccagcctgggaggtccctgagactctcctgtgcagcctctggattcaccttcagtagctttggcatgcactgggtccgccaggctccaggcaaggggctggagtgggtgatatttatatcatatgatggaagtgataaatactatgcagactccgtgaagggccgattcgccatctccagagacagttccaagaacacgctatatctgcaaatgaacagcctgagagctgaggacacggctgtgtattactgtgcgaaagaaaacggtattttgactgattcctacggtatggacgtctggggccaagggaccacggtcaccgtctcctca

SEQ ID NO: 192 - protein, 122 aa, synthetic construct
QVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSS

SEQ ID NO: 193 - protein, 8 aa, synthetic construct
GFTFSSFG

SEQ ID NO: 194 - protein, 8 aa, synthetic construct
ISYDGSDK

SEQ ID NO: 195 - protein, 15 aa, synthetic construct
AKENGILTDSYGMDV

SEQ ID NO: 196 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 197 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 198 - protein, 6 aa, synthetic construct
QSISSY

SEQ ID NO: 199 - skipped

SEQ ID NO: 200 - protein, 10 aa, synthetic construct
QQSYSTPPIT

SEQ ID NO: 201 - DNA, 372 bp, synthetic construct
  source 1..372: mol_type = other DNA
caggttcagctggtgcagtctggagctgaggtgaagaagcctggggcctctgtgaaggtctcctgcaaggcttctggttacacctttaccagctatggtatcagctgggtgcgacaggcccctggacaagggcttgagtggatgggatggatcagtgtttaccatggtaacacaaactatgcacagaagttccagggcagagtcaccatgaccacagacacatccacgagcacagcctacatggagctgaggagcctgagatctgacgacacggccgtgtattactgtgcgagagaggggtattacgatttttggagtggttattacccttttgactactggggccagggaaccctggtcaccgtctcctca

SEQ ID NO: 202 - protein, 124 aa, synthetic construct
QVQLVQSGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTLVTVSS

SEQ ID NO: 203 - protein, 8 aa, synthetic construct
GYTFTSYG

SEQ ID NO: 204 - protein, 8 aa, synthetic construct
ISVYHGNT

SEQ ID NO: 205 - protein, 17 aa, synthetic construct
AREGYYDFWSGYYPFDY

SEQ ID NO: 206 - DNA, 324 bp, synthetic construct; identical to SEQ ID NO: 196
  source 1..324: mol_type = other DNA

SEQ ID NO: 207 - protein, 108 aa, synthetic construct; identical to SEQ ID NO: 197

SEQ ID NO: 208 - protein, 6 aa, synthetic construct
QSISSY

SEQ ID NO: 209 - skipped

SEQ ID NO: 210 - protein, 10 aa, synthetic construct
QQSYSTPPIT

SEQ ID NO: 211 - DNA, 354 bp, synthetic construct
  source 1..354: mol_type = other DNA
gaggtgcagctggtggagtctgggggaggcttgatacaacctggagggtccctgagactctcctgtgaagcctctggattcaccttcagaaattatgaaatgaattgggtccgccaggctccagggaaggggctggagtgggtttcatatattagtagtagtggtaatatgaaagactacgcagagtctgtgaagggccgattcaccatctccagagacaatgtcaagaattcactgcagctgcaaatgaacagcctgagagtcgaggacacggctgtttattactgtgcgagagacgagtttccttacggaatggacgtctggggccaagggaccacggtcaccgtctcctca

SEQ ID NO: 212 - protein, 118 aa, synthetic construct
EVQLVESGGGLIQPGGSLRLSCEASGFTFRNYEMNWVRQAPGKGLEWVSYISSSGNMKDYAESVKGRFTISRDNVKNSLQLQMNSLRVEDTAVYYCARDEFPYGMDVWGQGTTVTVSS

SEQ ID NO: 213 - protein, 8 aa, synthetic construct
GFTFRNYE

SEQ ID NO: 214 - protein, 8 aa, synthetic construct
ISSSGNMK

SEQ ID NO: 215 - protein, 11 aa, synthetic construct
ARDEFPYGMDV

SEQ ID NO: 216 - DNA, 324 bp, synthetic construct; identical to SEQ ID NO: 196
  source 1..324: mol_type = other DNA

SEQ ID NO: 217 - protein, 108 aa, synthetic construct; identical to SEQ ID NO: 197

SEQ ID NO: 218 - protein, 6 aa, synthetic construct
QSISSY

SEQ ID NO: 219 - skipped

SEQ ID NO: 220 - protein, 10 aa, synthetic construct
QQSYSTPPIT

SEQ ID NO: 221 - DNA, 363 bp, synthetic construct
  source 1..363: mol_type = other DNA
gaagtgcagctggtggagtctgggggaggcttggttcagcctggcaggtccctgagactctcctgtgcagcctctggattcacctttgatgattatgccatgaactgggtccggcaagctccagggaagggcctggagtgggtctcaggtattagttggagtagtggtagcatggactatgcggactctgtgaagggccgattcaccatctccagagacaacgccaaaaactccctgtatctgcaaatgaacagtctgagaactgaggacacggccttatattactgtgcaaaagctagggaagttggagactactacggtatggacgtctggggccaagggaccacggtcaccgtctcctca

SEQ ID NO: 222 - protein, 121 aa, synthetic construct
EVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSS

SEQ ID NO: 223 - protein, 8 aa, synthetic construct
GFTFDDYA

SEQ ID NO: 224 - protein, 8 aa, synthetic construct
ISWSSGSM

SEQ ID NO: 225 - protein, 14 aa, synthetic construct
AKAREVGDYYGMDV

SEQ ID NO: 226 - DNA, 324 bp, synthetic construct; identical to SEQ ID NO: 196
  source 1..324: mol_type = other DNA

SEQ ID NO: 227 - protein, 108 aa, synthetic construct; identical to SEQ ID NO: 197

SEQ ID NO: 228 - protein, 6 aa, synthetic construct
QSISSY

SEQ ID NO: 229 - skipped

SEQ ID NO: 230 - protein, 10 aa, synthetic construct
QQSYSTPPIT

SEQ ID NO: 231 - DNA, 357 bp, synthetic construct
  source 1..357: mol_type = other DNA
gaagtgcagctggtggagtctgggggaggcttggtacagcctggcaggtccctgacactctcctgtgcagcctctggattcacctttgataattttggcatgcactgggtccggcaaggtccagggaagggcctggaatgggtctcaggtcttacttggaatagtggtgtcataggctatgcggactctgtgaagggccgattcaccatctccagagacaacgccaagaactccctgtatctgcaaatgaacagtctgagacctgaggacacggccttatattactgtgcaaaagatatacggaattacggcccctttgactactggggccagggaaccctggtcaccgtctcctca

SEQ ID NO: 232 - protein, 119 aa, synthetic construct
EVQLVESGGGLVQPGRSLTLSCAASGFTFDNFGMHWVRQGPGKGLEWVSGLTWNSGVIGYADSVKGRFTISRDNAKNSLYLQMNSLRPEDTALYYCAKDIRNYGPFDYWGQGTLVTVSS

SEQ ID NO: 233 - protein, 8 aa, synthetic construct
GFTFDNFG

SEQ ID NO: 234 - protein, 8 aa, synthetic construct
LTWNSGVI

SEQ ID NO: 235 - protein, 12 aa, synthetic construct
AKDIRNYGPFDY

SEQ ID NO: 236 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 237 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 238 - protein, 7 aa, synthetic construct
QSVSSSY

SEQ ID NO: 239 - skipped

SEQ ID NO: 240 - protein, 9 aa, synthetic construct
QQYGSSPWT

SEQ ID NO: 241 - DNA, 357 bp, synthetic construct
  source 1..357: mol_type = other DNA
gaggtgcagctggtggagtctgggggaggcttagtacagcctggagggtccctaagactctcctgtgcagcctctggattcaccttcaatatttttgaaatgaactgggtccgccaggctccagggaaggggctggagtggatttcctacattagtagtcgtggaactaccacatactacgcagactctgtgaggggccgattcaccatctccagagacaacgccaagaactcactgtatctgcaaatgaacagcctgagagccgaggacacggctgtttattactgtgcgagagattatgaagcaacaatcccttttgacttctggggccagggaaccctggtcaccgtctcctca

SEQ ID NO: 242 - protein, 119 aa, synthetic construct
EVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSS

SEQ ID NO: 243 - protein, 8 aa, synthetic construct
GFTFNIFE

SEQ ID NO: 244 - protein, 8 aa, synthetic construct
ISSRGTTT

SEQ ID NO: 245 - protein, 12 aa, synthetic construct
ARDYEATIPFDF

SEQ ID NO: 246 - DNA, 324 bp, synthetic construct; identical to SEQ ID NO: 196
  source 1..324: mol_type = other DNA

SEQ ID NO: 247 - protein, 108 aa, synthetic construct; identical to SEQ ID NO: 197

SEQ ID NO: 248 - protein, 6 aa, synthetic construct
QSISSY

SEQ ID NO: 249 - skipped

SEQ ID NO: 250 - protein, 10 aa, synthetic construct
QQSYSTPPIT

SEQ ID NO: 251 - DNA, 363 bp, synthetic construct
  source 1..363: mol_type = other DNA
gaggtgcagctggtggagtctgggggaagtgtggtacggcctggggggtccctgagactctcctgtgaagcctctggattcacctttgatgattatggcatgagctgggtccgccaagatccagggaaggggctggagtgggtctctggtattaattggaatggtgatagaacaaattatgcagactctgtgaagggccgattcatcatttccagagacaacgccaagaactctgtgtatctacaaatgaacagtctgagagcggaggactcggccttgtatcactgtgcgagagatcagggactcggagtggcagctacccttgactactggggccagggaaccctggtcaccgtctcctca

SEQ ID NO: 252 - protein, 121 aa, synthetic construct
EVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTLVTVSS

SEQ ID NO: 253 - protein, 8 aa, synthetic construct
GFTFDDYG

SEQ ID NO: 254 - protein, 8 aa, synthetic construct
INWNGDRT

SEQ ID NO: 255 - protein, 14 aa, synthetic construct
ARDQGLGVAATLDY

SEQ ID NO: 256 - DNA, 324 bp, synthetic construct; identical to SEQ ID NO: 196
  source 1..324: mol_type = other DNA

SEQ ID NO: 257 - protein, 108 aa, synthetic construct; identical to SEQ ID NO: 197

SEQ ID NO: 258 - protein, 6 aa, synthetic construct
QSISSY

SEQ ID NO: 259 - skipped

SEQ ID NO: 260 - protein, 10 aa, synthetic construct
QQSYSTPPIT

SEQ ID NO: 261 - DNA, 384 bp, synthetic construct
  source 1..384: mol_type = other DNA
gaggtgcagctggtggagtctgggggaggcttggtacagcctggagggtccctgagactctcctgtgcagcctctggattcaccgtcagtaattatgaaatgaactgggtccgccaggctccagggaaggggctggagtgggtttcatacattagtagtagtaccagtaacatatactacgcagactctgtgaagggccgattcaccatctccagagacaacgccgagaactcactgtatctgcagatgaacagcctgagagtcgaggacacggctgtttattactgtgtgagagatgggattgtagtagttccagttggtcgtggatactactattacggtttggacgtctggggccaagggaccacggtcaccgtctcctca

SEQ ID NO: 262 - protein, 128 aa, synthetic construct
EVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSS

SEQ ID NO: 263 - protein, 8 aa, synthetic construct
GFTVSNYE

SEQ ID NO: 264 - protein, 8 aa, synthetic construct
ISSSTSNI

SEQ ID NO: 265 - protein, 21 aa, synthetic construct
VRDGIVVVPVGRGYYYYGLDV

SEQ ID NO: 266 - DNA, 324 bp, synthetic construct; identical to SEQ ID NO: 196
  source 1..324: mol_type = other DNA

SEQ ID NO: 267 - protein, 108 aa, synthetic construct; identical to SEQ ID NO: 197

SEQ ID NO: 268 - protein, 6 aa, synthetic construct
QSISSY

SEQ ID NO: 269 - skipped

SEQ ID NO: 270 - protein, 10 aa, synthetic construct
QQSYSTPPIT

SEQ ID NO: 271 - DNA, 378 bp, synthetic construct
  source 1..378: mol_type = other DNA
caggtgcagctggtggagtctgggggaggcgtggtccagcctggaaggtccctgagactctcctgcgcagcctctggattcccctttagtaattatgtcatgtattgggtccgccaggctccaggcaaggggctggagtgggtggctcttattttttttgacggaaagaaaaactatcatgcagactccgtgaagggccgattcaccataaccagagacaattccaaaaatatgttatatctgcaaatgaacagcctgagacctgaggacgcggctgtgtattactgtgcgaaaatccattgtcctaatggtgtatgttacaaggggtattacggaatggacgtctggggccaagggaccacggtcaccgtctcctca

SEQ ID NO: 272 - protein, 126 aa, synthetic construct
QVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSS

SEQ ID NO: 273 - protein, 8 aa, synthetic construct
GFPFSNYV

SEQ ID NO: 274 - protein, 8 aa, synthetic construct
IFFDGKKN

SEQ ID NO: 275 - protein, 19 aa, synthetic construct
AKIHCPNGVCYKGYYGMDV

SEQ ID NO: 276 - DNA, 324 bp, synthetic construct; identical to SEQ ID NO: 196
  source 1..324: mol_type = other DNA

SEQ ID NO: 277 - protein, 108 aa, synthetic construct; identical to SEQ ID NO: 197

SEQ ID NO: 278 - protein, 6 aa, synthetic construct
QSISSY

SEQ ID NO: 279 - skipped

SEQ ID NO: 280 - protein, 10 aa, synthetic construct
QQSYSTPPIT

SEQ ID NO: 281 - DNA, 375 bp, synthetic construct
  source 1..375: mol_type = other DNA
gaggtgcagctggtggagtctgggggaggcttggtccagcctggggggtccctaagactctcctgtgcagcctctggattcacctttagtaactattggatgaactgggtccgccaggctccagggaagggactggagtgggtggccaatataaaagaagatggaggtaagaaattgtatgtggactctgtgaagggccgattcaccatctccagagacaacgccaagaactcactgtttctgcaaatgaacagcctgagagccgaggacacggctgtgtattattgtgcgagagaagatacaactttggttgtggactactactactacggtatggacgtctggggccaagggaccacggtcaccgtctcctca

SEQ ID NO: 282 - protein, 125 aa, synthetic construct
EVQLVESGGGLVQPGGSLRLSCAASGFTFSNYWMNWVRQAPGKGLEWVANIKEDGGKKLYVDSVKGRFTISRDNAKNSLFLQMNSLRAEDTAVYYCAREDTTLVVDYYYYGMDVWGQGTTVTVSS

SEQ ID NO: 283 - protein, 8 aa, synthetic construct
GFTFSNYW

SEQ ID NO: 284 - protein, 8 aa, synthetic construct
IKEDGGKK

SEQ ID NO: 285 - protein, 18 aa, synthetic construct
AREDTTLVVDYYYYGMDV

SEQ ID NO: 286 - DNA, 324 bp, synthetic construct; identical to SEQ ID NO: 196
  source 1..324: mol_type = other DNA

SEQ ID NO: 287 - protein, 108 aa, synthetic construct; identical to SEQ ID NO: 197

SEQ ID NO: 288 - protein, 6 aa, synthetic construct
QSISSY

SEQ ID NO: 289 - skipped

SEQ ID NO: 290 - protein, 10 aa, synthetic construct
QQSYSTPPIT

SEQ ID NO: 291 - DNA, 366 bp, synthetic construct
  source 1..366: mol_type = other DNA
caggtccagctggtgcagtctggggctgaggtgaagaagcctgggtcctcggtgaaggtctcctgcaaggcttctggaggcaccttcaacacctatgctatcacctgggtgcgacaggcccctggacaagggcttgaatggatggggggaatcatccctatctctggcatagcagagtacgcacagaagttccagggcagagtcacgatcaccacggatgactcctcgaccacagcctacatggaactgaacagtctgagatctgaggacacggccgtgtattactgtgcgagctggaactacgcactctactacttctacggtatggacgtctggggccgagggaccacggtcaccgtctcctca

SEQ ID NO: 292 - protein, 122 aa, synthetic construct
QVQLVQSGAEVKKPGSSVKVSCKASGGTFNTYAITWVRQAPGQGLEWMGGIIPISGIAEYAQKFQGRVTITTDDSSTTAYMELNSLRSEDTAVYYCASWNYALYYFYGMDVWGRGTTVTVSS

SEQ ID NO: 293 - protein, 8 aa, synthetic construct
GGTFNTYA

SEQ ID NO: 294 - protein, 8 aa, synthetic construct
IIPISGIA

SEQ ID NO: 295 - protein, 15 aa, synthetic construct
ASWNYALYYFYGMDV

SEQ ID NO: 296 - DNA, 324 bp, synthetic construct; identical to SEQ ID NO: 236
  source 1..324: mol_type = other DNA

SEQ ID NO: 297 - protein, 108 aa, synthetic construct; identical to SEQ ID NO: 237

SEQ ID NO: 298 - protein, 7 aa, synthetic construct
QSVSSSY

SEQ ID NO: 299 - skipped

SEQ ID NO: 300 - protein, 9 aa, synthetic construct
QQYGSSPWT

SEQ ID NO: 301 - DNA, 369 bp, synthetic construct
  source 1..369: mol_type = other DNA
caggtgcagctggtggagtctgggggaggcttggtcaagcctggagggtccctgagactctcctgtgcagcctctggattcaccttcagtgtctattacatgaactggatccgccaggctccagggaagggcctggagtgggtttcatacattagtagtagtggtagtaccatatactacgcagactctgtgaagggccgattcaccatctccagggacaacgccaagaactcactgtatctccaaatgaacagtctgagagccgaggacacggccgtatattactgtgggagagaagggtatagtgggacttattcttattacggtatggacgtctggggccaagggaccacggtcaccgtctcctca

SEQ ID NO: 302 - protein, 123 aa, synthetic construct
QVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSS

SEQ ID NO: 303 - protein, 8 aa, synthetic construct
GFTFSVYY

SEQ ID NO: 304 - protein, 8 aa, synthetic construct
ISSSGSTI

SEQ ID NO: 305 - protein, 16 aa, synthetic construct
GREGYSGTYSYYGMDV

SEQ ID NO: 306 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagttcctgatctatttgggttctaatcgggcctccggggtccctgacaggttcagtggcagtggatcaggcacagattttacactgaaaatcaacagagtggaggctgaggatgttggggtttattactgcatgcaagctctacaaactccgtacacttttggccaggggaccaagctggagatcaaa

SEQ ID NO: 307 - protein, 112 aa, synthetic construct
DIVMTQSPLSLPVTPGEPASISCRSSQSLLHSNGYNYLDWYLQKPGQSPQFLIYLGSNRASGVPDRFSGSGSGTDFTLKINRVEAEDVGVYYCMQALQTPYTFGQGTKLEIK

SEQ ID NO: 308 - protein, 11 aa, synthetic construct
QSLLHSNGYNY

SEQ ID NO: 309 - skipped

SEQ ID NO: 310 - protein, 9 aa, synthetic construct
MQALQTPYT

SEQ ID NO: 311 - DNA, 366 bp, synthetic construct
  source 1..366: mol_type = other DNA
gaagtgcagctggtggagtctgggggagggttggtacagcctggcaggtccctgagactctcctgtgcagtctctggattcacctttgatgattatgccatgcactgggtccggcaagctccagggaagggcctggagtgggtctcaggtattagttggaatagtggtaccagaggatatgcggactctgtgaagggccgattcaccatctccagagacaacgccaagaactccctgtatctgcaaatgaacagtctgagaggtgaggacacggccttgtattactgtgtaaaagatattacgatatcccccaactactacggtatggacgtctggggccaagggaccacggtcaccgtctcctca

SEQ ID NO: 312 - protein, 122 aa, synthetic construct
EVQLVESGGGLVQPGRSLRLSCAVSGFTFDDYAMHWVRQAPGKGLEWVSGISWNSGTRGYADSVKGRFTISRDNAKNSLYLQMNSLRGEDTALYYCVKDITISPNYYGMDVWGQGTTVTVSS

SEQ ID NO: 313 - protein, 8 aa, synthetic construct
GFTFDDYA

SEQ ID NO: 314 - protein, 8 aa, synthetic construct
ISWNSGTR

SEQ ID NO: 315 - protein, 15 aa, synthetic construct
VKDITISPNYYGMDV

SEQ ID NO: 316 - DNA, 321 bp, synthetic construct
  source 1..321: mol_type = other DNA
gacatccagatgacccagtctccatcctccctgtctgcatctgtaggagacagagtcaccatcacttgccgggcgagtcaggacattagccattattcagcctggtatcagcagaaaccagggaaacttcctaacctcctgatctatgctgcatccactttgcaatcaggggtcccatctcggttcagtggcagtggatctgggacagatttctctctcaccaccagcagcctgcagcctgaagatgttgcaacttattactgtcaaaagtataacagtgtccctctcactttcggcggagggaccaaggtggagatcaaa

SEQ ID NO: 317 - protein, 107 aa, synthetic construct
DIQMTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGGTKVEIK

SEQ ID NO: 318 - protein, 6 aa, synthetic construct
QDISHY

SEQ ID NO: 319 - skipped

SEQ ID NO: 320 - protein, 9 aa, synthetic construct
QKYNSVPLT

SEQ ID NO: 321 - RNA, 16 nt, synthetic construct
  source 1..16: mol_type = other RNA
gttttagagctatgct

SEQ ID NO: 322 - RNA, 22 nt, synthetic construct
  source 1..22: mol_type = other RNA
gttttagagctatgctgttttg

SEQ ID NO: 323 - RNA, 67 nt, synthetic construct
  source 1..67: mol_type = other RNA
agcatagcaagttaaaataaggctagtccgttatcaacttgaaaaagtggcaccgagtcggtgcttt

SEQ ID NO: 324 - RNA, 72 nt, synthetic construct
  source 1..72: mol_type = other RNA
aaacagcatagcaagttaaaataaggctagtccgttatcaacttgaaaaagtggcaccgagtcggtgctttt

SEQ ID NO: 325 - RNA, 82 nt, synthetic construct
  source 1..82: mol_type = other RNA
gttggaaccattcaaaacagcatagcaagttaaaataaggctagtccgttatcaacttgaaaaagtggcaccgagtcggtgc

SEQ ID NO: 326 - protein, 118 aa, synthetic construct
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 327 - protein, 107 aa, synthetic construct
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELK

SEQ ID NO: 328 - protein, 214 aa, synthetic construct
DIQMTQSPSSLSASVGDRVTITCRASQGISNYLAWYQQKPGKVPNLLIYAASTLQSGVPSRFSGSGSGTDFTLTISSLQPEDVATYYCQKYNSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 329 - protein, 228 aa, synthetic construct
EVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 330 - protein, 214 aa, synthetic construct
DIQMTQSPSSLSASIGDRVTITCRASQGISNYLAWYQQKPGKVPKLLIYAASTLQSGVPSRFSGSGSGTDFTLTISSLQPEDVATYYCQNHNSVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 331 - protein, 228 aa, synthetic construct
EVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 332 - protein, 214 aa, synthetic construct
DIQMTQSPSSLSASVGDRVTITCRASQSIRNVLGWFQQKPGKAPQRLIYAASSLQSGVPSRFSGSGSGTEFTLTISSLQPEDFATYYCLQHNFYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 333 - protein, 229 aa, synthetic construct
QVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 334 - protein, 214 aa, synthetic construct
EIVLTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 335 - protein, 230 aa, synthetic construct
EVQLVESGGGLVQPGRSLRLSCAASGFTFDDKAMHWVRQVPGKGLEWISGISWNSGTIGYADSVKGRFIISRDNAKNSLYLQMNSLRAEDTALYYCAKDGDTSGWYWYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 336 - protein, 214 aa, synthetic construct
DIQLTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 337 - protein, 227 aa, synthetic construct
QVQLVESGGGVVQPGRSLRLSCIASGFTFSVYGIHWVRQAPGKGLEWMAVISHDGNIKHYADSVKGRFTISRDNSKNTLYLQINSLRTEDTAVYYCAKDTWNSLDTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 338 - protein, 214 aa, synthetic construct
AIQMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 339 - protein, 227 aa, synthetic construct
QVTLRESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 340 - protein, 214 aa, synthetic construct
EIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 341 - protein, 223 aa, synthetic construct
EVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 342 - protein, 214 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 343 - protein, 232 aa, synthetic construct
EVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 344 - protein, 214 aa, synthetic construct
DIQMTQSPSSLSASVGDRVTITCRASQSISSYLNWYQQKPGKAPKVLIYAASSLQSGVPSRFSGSGSGTDFTLTISSLQPEDFATYYCQQSYSIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 345 - protein, 234 aa, synthetic construct
EVQLVESGGGLVQPGRSLRLSCAASGFTFDDYAMHWVRQAPGKGLEWVSGISWNSGYIGYADSVKGRFTISRDNAENSLHLQMNSLRAEDTALYYCARGGSTLVRGVKGGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 346 - protein, 214 aa, synthetic construct
DIQMTQSPSSLSASVGDRVTITCRASQSIDRYLNWYRQKPGKAPKLLIYTTSSLQSGVPSRFSGSGSGTDFTLTLSSLQPEDFATYYCQQSYSPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 347 - protein, 224 aa, synthetic construct
QVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 348 - protein, 214 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKADSLPYAFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 349 - protein, 232 aa, synthetic construct
EVQLVESGGGLVQPGGSLRLSCTASGFTFSNYWMTWVRQAPGKGLEWVANIKEDGSEKEYVDSVKGRFTISRDNAKNSLYLQMNSLRGEDTAVYYCARDGEQLVDYYYYYVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 350 - protein, 214 aa, synthetic construct
DIQMTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 351 - protein, 228 aa, synthetic construct
EVQLVESGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 352 - protein, 215 aa, synthetic construct
EIVMTQSPATLSVSPGERATLSCRASQTVSSNLAWYQQKPGQAPRLLIYGSSSRATGIPARFSGSGSGTEFTLTISSLQSEDFAVYYCQQYNNWPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 353 - protein, 229 aa, synthetic construct
EVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 354 - protein, 214 aa, synthetic construct
DIQMTQSPSSVSASVGDRVTITCRASQGIASWLAWYQQKPGKAPELLIYAASSLQGGVPSRFSGSGSGTDFTLTISSLQPEDFAIYYCQQANYFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 355 - protein, 231 aa, synthetic construct
QITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 356 - protein, 214 aa, synthetic construct
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 357 - protein, 228 aa, synthetic construct
EVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 358 - protein, 214 aa, synthetic construct
EIVMTQSPATLSVSPGERATLSCRASQSVSINLAWYQQKPGQAPRLLIFVASTRATGIPARFSGSGSGTEFTLTISSLQSEDFATYYCQQYDIWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 359 - protein, 223 aa, synthetic construct
QVQLVESGGGLVKPGGSLRLSCAASGFTFSDYFMSWIRQAPGKGLEWVSYISSTGSTINYADSVKGRFTISRDNVKNSLYLQMTSLRVEDTAVYYCTRDNWNYEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 360 - protein, 214 aa, synthetic construct
DIQMTQSPSSLSASVGDRVTINCRASQGIRNDLGWYQQKPGKAPKRLIYAASSLQSGVPLRFSGSGSGTEFTLTINNLQPEDFATYYCLSHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 361 - protein, 230 aa, synthetic construct
QLQLQESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 362 - protein, 214 aa, synthetic construct
DIQMTQSPPSVSASVGDRVTITCRASQGISSWLAWYQQKPGKAPKLLIYAASSLQSGVPSRFSGSGSGTDFTLTISSLQPEDFATYYCQQANSFPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 363 - protein, 225 aa, synthetic construct
QVQLVQSGAEVKKPGSSVRVSCKASRGTFSSYAISWVRQAPGQGLEWMGGIIPIFGTANYAQKFLARVTITADESTSTAYMELSSLRSEDTAVYYCAREKGWNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 364 - protein, 219 aa, synthetic construct
DIVMTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 365 - protein, 230 aa, synthetic construct
QVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 366 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 367 - protein, 229 aa, synthetic construct
QVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 368 - protein, 215 aa, synthetic construct; identical to SEQ ID NO: 366

SEQ ID NO: 369 - protein, 231 aa, synthetic construct
QVQLVQSGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 370 - protein, 215 aa, synthetic construct; identical to SEQ ID NO: 366

SEQ ID NO: 371 - protein, 225 aa, synthetic construct
EVQLVESGGGLIQPGGSLRLSCEASGFTFRNYEMNWVRQAPGKGLEWVSYISSSGNMKDYAESVKGRFTISRDNVKNSLQLQMNSLRVEDTAVYYCARDEFPYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 372 - protein, 215 aa, synthetic construct; identical to SEQ ID NO: 366

SEQ ID NO: 373 - protein, 228 aa, synthetic construct
EVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 374 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 375 - protein, 226 aa, synthetic construct
EVQLVESGGGLVQPGRSLTLSCAASGFTFDNFGMHWVRQGPGKGLEWVSGLTWNSGVIGYADSVKGRFTISRDNAKNSLYLQMNSLRPEDTALYYCAKDIRNYGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 376 - protein, 215 aa, synthetic construct; identical to SEQ ID NO: 366

SEQ ID NO: 377 - protein, 226 aa, synthetic construct
EVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 378 - protein, 215 aa, synthetic construct; identical to SEQ ID NO: 366

SEQ ID NO: 379 - protein, 228 aa, synthetic construct
EVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 380 - protein, 215 aa, synthetic construct; identical to SEQ ID NO: 366

SEQ ID NO: 381 - protein, 235 aa, synthetic construct
EVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 382 - protein, 215 aa, synthetic construct; identical to SEQ ID NO: 366

SEQ ID NO: 383 - protein, 233 aa, synthetic construct
QVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 384 - protein, 215 aa, synthetic construct; identical to SEQ ID NO: 366

SEQ ID NO: 385 - protein, 232 aa, synthetic construct
EVQLVESGGGLVQPGGSLRLSCAASGFTFSNYWMNWVRQAPGKGLEWVANIKEDGGKKLYVDSVKGRFTISRDNAKNSLFLQMNSLRAEDTAVYYCAREDTTLVVDYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 386 - protein, 215 aa, synthetic construct; identical to SEQ ID NO: 374

SEQ ID NO: 387 - protein, 229 aa, synthetic construct
QVQLVQSGAEVKKPGSSVKVSCKASGGTFNTYAITWVRQAPGQGLEWMGGIIPISGIAEYAQKFQGRVTITTDDSSTTAYMELNSLRSEDTAVYYCASWNYALYYFYGMDVWGRGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 388 - protein, 219 aa, synthetic construct
DIVMTQSPLSLPVTPGEPASISCRSSQSLLHSNGYNYLDWYLQKPGQSPQFLIYLGSNRASGVPDRFSGSGSGTDFTLKINRVEAEDVGVYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 389 - protein, 230 aa, synthetic construct
QVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 390 - protein, 214 aa, synthetic construct
DIQMTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 391 - protein, 229 aa, synthetic construct
EVQLVESGGGLVQPGRSLRLSCAVSGFTFDDYAMHWVRQAPGKGLEWVSGISWNSGTRGYADSVKGRFTISRDNAKNSLYLQMNSLRGEDTALYYCVKDITISPNYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 392 - protein, 243 aa, synthetic construct
DIQMTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIKGGGGSGGGGSGGGGSEVQLVESGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTLVTVSS

SEQ ID NO: 393 - protein, 245 aa, synthetic construct
EIVMTQSPATLSVSPGERATLSCRASQTVSSNLAWYQQKPGQAPRLLIYGSSSRATGIPARFSGSGSGTEFTLTISSLQSEDFAVYYCQQYNNWPPYTFGQGTKLEIKGGGGSGGGGSGGGGSEVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSS

SEQ ID NO: 394 - protein, 246 aa, synthetic construct
DIQMTQSPSSVSASVGDRVTITCRASQGIASWLAWYQQKPGKAPELLIYAASSLQGGVPSRFSGSGSGTDFTLTISSLQPEDFAIYYCQQANYFPWTFGQGTKVEIKGGGGSGGGGSGGGGSQITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSS

SEQ ID NO: 395 - protein, 243 aa, synthetic construct
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVEIKGGGGSGGGGSGGGGSEVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTLVTVSS

SEQ ID NO: 396 - protein, 238 aa, synthetic construct
EIVMTQSPATLSVSPGERATLSCRASQSVSINLAWYQQKPGQAPRLLIFVASTRATGIPARFSGSGSGTEFTLTISSLQSEDFATYYCQQYDIWPYTFGQGTKLEIKGGGGSGGGGSGGGGSQVQLVESGGGLVKPGGSLRLSCAASGFTFSDYFMSWIRQAPGKGLEWVSYISSTGSTINYADSVKGRFTISRDNVKNSLYLQMTSLRVEDTAVYYCTRDNWNYEYWGQGTLVTVSS

SEQ ID NO: 397 - protein, 245 aa, synthetic construct
DIQMTQSPSSLSASVGDRVTINCRASQGIRNDLGWYQQKPGKAPKRLIYAASSLQSGVPLRFSGSGSGTEFTLTINNLQPEDFATYYCLSHNSYPWTFGQGTKVEIKGGGGSGGGGSGGGGSQLQLQESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSS

SEQ ID NO: 398 - protein, 240 aa, synthetic construct
DIQMTQSPPSVSASVGDRVTITCRASQGISSWLAWYQQKPGKAPKLLIYAASSLQSGVPSRFSGSGSGTDFTLTISSLQPEDFATYYCQQANSFPRTFGQGTKVEIKGGGGSGGGGSGGGGSQVQLVQSGAEVKKPGSSVRVSCKASRGTFSSYAISWVRQAPGQGLEWMGGIIPIFGTANYAQKFLARVTITADESTSTAYMELSSLRSEDTAVYYCAREKGWNYFDYWGQGTLVTVSS

SEQ ID NO: 399 - protein, 250 aa, synthetic construct
DIVMTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKLEIKGGGGSGGGGSGGGGSQVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSS

SEQ ID NO: 400 - protein, 245 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSS

SEQ ID NO: 401 - protein, 247 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVQSGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTLVTVSS

SEQ ID NO: 402 - protein, 241 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLIQPGGSLRLSCEASGFTFRNYEMNWVRQAPGKGLEWVSYISSSGNMKDYAESVKGRFTISRDNVKNSLQLQMNSLRVEDTAVYYCARDEFPYGMDVWGQGTTVTVSS

SEQ ID NO: 403 - protein, 249 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSS

SEQ ID NO: 404 - protein, 248 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTFSNYWMNWVRQAPGKGLEWVANIKEDGGKKLYVDSVKGRFTISRDNAKNSLFLQMNSLRAEDTAVYYCAREDTTLVVDYYYYGMDVWGQGTTVTVSS

SEQ ID NO: 405 - protein, 242 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSS

SEQ ID NO: 406 - protein, 244 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTLVTVSS

SEQ ID NO: 407 - protein, 251 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSS

SEQ ID NO: 408 - protein, 244 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSS

SEQ ID NO: 409 - protein, 242 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSEVQLVESGGGLVQPGRSLTLSCAASGFTFDNFGMHWVRQGPGKGLEWVSGLTWNSGVIGYADSVKGRFTISRDNAKNSLYLQMNSLRPEDTALYYCAKDIRNYGPFDYWGQGTLVTVSS

SEQ ID NO: 410 - protein, 245 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSQVQLVQSGAEVKKPGSSVKVSCKASGGTFNTYAITWVRQAPGQGLEWMGGIIPISGIAEYAQKFQGRVTITTDDSSTTAYMELNSLRSEDTAVYYCASWNYALYYFYGMDVWGRGTTVTVSS

SEQ ID NO: 411 - protein, 243 aa, synthetic construct
DIQMTQSPSSLSASIGDRVTITCRASQGISNYLAWYQQKPGKVPKLLIYAASTLQSGVPSRFSGSGSGTDFTLTISSLQPEDVATYYCQNHNSVPLTFGGGTKVEIKGGGGSGGGGSGGGGSEVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTLVTVSS

SEQ ID NO: 412 - protein, 243 aa, synthetic construct
DIQMTQSPSSLSASVGDRVTITCRASQGISNYLAWYQQKPGKVPNLLIYAASTLQSGVPSRFSGSGSGTDFTLTISSLQPEDVATYYCQKYNSAPLTFGGGTKVEIKGGGGSGGGGSGGGGSEVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTLVTVSS

SEQ ID NO: 413 - protein, 250 aa, synthetic construct
DIVMTQSPLSLPVTPGEPASISCRSSQSLLHSNGYNYLDWYLQKPGQSPQFLIYLGSNRASGVPDRFSGSGSGTDFTLKINRVEAEDVGVYYCMQALQTPYTFGQGTKLEIKGGGGSGGGGSGGGGSQVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSS

SEQ ID NO: 414 - protein, 244 aa, synthetic construct
DIQMTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGGTKVEIKGGGGSGGGGSGGGGSEVQLVESGGGLVQPGRSLRLSCAVSGFTFDDYAMHWVRQAPGKGLEWVSGISWNSGTRGYADSVKGRFTISRDNAKNSLYLQMNSLRGEDTALYYCVKDITISPNYYGMDVWGQGTTVTVSS

SEQ ID NO: 415 - protein, 239 aa, synthetic construct
DIQMTQSPSSLSASVGDRVTITCRASQSIDRYLNWYRQKPGKAPKLLIYTTSSLQSGVPSRFSGSGSGTDFTLTLSSLQPEDFATYYCQQSYSPPLTFGGGTKVEIKGGGGSGGGGSGGGGSQVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSS

SEQ ID NO: 416 - protein, 247 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKADSLPYAFGQGTKLEIKGGGGSGGGGSGGGGSEVQLVESGGGLVQPGGSLRLSCTASGFTFSNYWMTWVRQAPGKGLEWVANIKEDGSEKEYVDSVKGRFTISRDNAKNSLYLQMNSLRGEDTAVYYCARDGEQLVDYYYYYVMDVWGQGTTVTVSS

SEQ ID NO: 417 - protein, 249 aa, synthetic construct
DIQMTQSPSSLSASVGDRVTITCRASQSISSYLNWYQQKPGKAPKVLIYAASSLQSGVPSRFSGSGSGTDFTLTISSLQPEDFATYYCQQSYSIPLTFGGGTKVEIKGGGGSGGGGSGGGGSEVQLVESGGGLVQPGRSLRLSCAASGFTFDDYAMHWVRQAPGKGLEWVSGISWNSGYIGYADSVKGRFTISRDNAENSLHLQMNSLRAEDTALYYCARGGSTLVRGVKGGYYGMDVWGQGTTVTVSS

SEQ ID NO: 418 - protein, 238 aa, synthetic construct
EIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIKGGGGSGGGGSGGGGSEVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSS

SEQ ID NO: 419 - protein, 247 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKLEIKGGGGSGGGGSGGGGSEVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSS

SEQ ID NO: 420 - protein, 242 aa, synthetic construct
DIQLTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIKGGGGSGGGGSGGGGSQVQLVESGGGVVQPGRSLRLSCIASGFTFSVYGIHWVRQAPGKGLEWMAVISHDGNIKHYADSVKGRFTISRDNSKNTLYLQINSLRTEDTAVYYCAKDTWNSLDTFDIWGQGTMVTVSS

SEQ ID NO: 421 - protein, 242 aa, synthetic construct
AIQMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVDIKGGGGSGGGGSGGGGSQVTLRESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSS

SEQ ID NO: 422 - protein, 245 aa, synthetic construct
EIVLTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIKGGGGSGGGGSGGGGSEVQLVESGGGLVQPGRSLRLSCAASGFTFDDKAMHWVRQVPGKGLEWISGISWNSGTIGYADSVKGRFIISRDNAKNSLYLQMNSLRAEDTALYYCAKDGDTSGWYWYGLDVWGQGTTVTVSS

SEQ ID NO: 423 - protein, 244 aa, synthetic construct
DIQMTQSPSSLSASVGDRVTITCRASQSIRNVLGWFQQKPGKAPQRLIYAASSLQSGVPSRFSGSGSGTEFTLTISSLQPEDFATYYCLQHNFYPLTFGGGTKVEIKGGGGSGGGGSGGGGSQVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTLVTVSS

SEQ ID NO: 424 - protein, 107 aa, synthetic construct [sequence omitted]

SEQ ID NO: 425 - protein, 107 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 426 - protein, 5 aa, synthetic construct
GGGGS

SEQ ID NO: 427 - RNA, 77 nt, synthetic construct
  source 1..77: mol_type = other RNA
gttttagagctagaaatagcaagttaaaataaggctagtccgttatcaacttgaaaaagtggcaccgagtcggtgct

SEQ ID NO: 428 - protein, 16 aa, synthetic construct
KRPAATKKAGQAKKKK

SEQ ID NO: 429 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 430 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 431 - RNA, 83 nt, synthetic construct
  source 1..83: mol_type = other RNA
gttttagagctagaaatagcaagttaaaataaggctagtccgttatcaacttgaaaaagtggcaccgagtcggtgcttttttt

SEQ ID NO: 432 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 433 - RNA, 92 nt, synthetic construct
  source 1..92: mol_type = other RNA
gtttaagagctatgctggaaacagcatagcaagtttaaataaggctagtccgttatcaacttgaaaaagtggcaccgagtcggtgctttttt

SEQ ID NO: 434 - RNA, 68 nt, synthetic construct
  source 1..68: mol_type = other RNA
gttttagagctagaaatagcaagttaaaataaggctagtccgttatcaacttggcaccgagtcggtgc

SEQ ID NO: 435 - RNA, 100 nt, synthetic construct
  source 1..100: mol_type = other RNA
  misc_difference 1..20: note = n = any natural or non-natural nucleotide
nnnnnnnnnnnnnnnnnnnngttttagagctagaaatagcaagttaaaataaggctagtccgttatcaacttgaaaaagtggcaccgagtcggtgctttt

SEQ ID NO: 436 - protein, 7 aa, synthetic construct
PKKKRKV

SEQ ID NO: 437 - protein, 7 aa, synthetic construct
PKKKRRV

SEQ ID NO: 438 - skipped

SEQ ID NO: 439 - protein, 5 aa, synthetic construct
LLQGG

SEQ ID NO: 440 - protein, 4 aa, synthetic construct
LLQG

SEQ ID NO: 441 - protein, 6 aa, synthetic construct
LSLSQG

SEQ ID NO: 442 - protein, 8 aa, synthetic construct
GGGLLQGG

SEQ ID NO: 443 - protein, 5 aa, synthetic construct
GLLQG

SEQ ID NO: 444 - skipped

SEQ ID NO: 445 - protein, 10 aa, synthetic construct
GSPLAQSHGG

SEQ ID NO: 446 - protein, 7 aa, synthetic construct
GLLQGGG

SEQ ID NO: 447 - protein, 6 aa, synthetic construct
GLLQGG

SEQ ID NO: 448 - protein, 4 aa, synthetic construct
GLLQ

SEQ ID NO: 449 - protein, 8 aa, synthetic construct
LLQLLQGA

SEQ ID NO: 450 - protein, 5 aa, synthetic construct
LLQGA

SEQ ID NO: 451 - protein, 7 aa, synthetic construct
LLQYQGA

SEQ ID NO: 452 - protein, 6 aa, synthetic construct
LLQGSG

SEQ ID NO: 453 - protein, 6 aa, synthetic construct
LLQYQG

SEQ ID NO: 454 - protein, 7 aa, synthetic construct
LLQLLQG

SEQ ID NO: 455 - protein, 5 aa, synthetic construct
SLLQG

SEQ ID NO: 456 - protein, 5 aa, synthetic construct
LLQLQ

SEQ ID NO: 457 - protein, 6 aa, synthetic construct
LLQLLQ

SEQ ID NO: 458 - protein, 5 aa, synthetic construct
LLQGR

SEQ ID NO: 459 - protein, 111 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPLLQGSG

SEQ ID NO: 460 - protein, 700 aa, synthetic construct
HHHHHHEQKLISEEDLGGEQKLISEEDLCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 461 - protein, 28 aa, synthetic construct
EQKLISEEDLGGEQKLISEEDLHHHHHH

SEQ ID NO: 462 - protein, 121 aa, synthetic construct
EVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTTVTVSS

SEQ ID NO: 463 - protein, 121 aa, synthetic construct
EVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTTVTVSS

SEQ ID NO: 464 - protein, 122 aa, synthetic construct
EVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTMVTVSS

SEQ ID NO: 465 - protein, 107 aa, synthetic construct
DIVMTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIK

SEQ ID NO: 466 - protein, 107 aa, synthetic construct
DIQMTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 467 - protein, 120 aa, synthetic construct
QVQLVESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSS

SEQ ID NO: 468 - protein, 107 aa, synthetic construct
DILMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVEIK

SEQ ID NO: 469 - protein, 107 aa, synthetic construct
DIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIK

SEQ ID NO: 470 - protein, 125 aa, synthetic construct
QVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSS

SEQ ID NO: 471 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKVEIK

SEQ ID NO: 472 - protein, 117 aa, synthetic construct
EVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSS

SEQ ID NO: 473 - protein, 121 aa, synthetic construct
EVQLVQSGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTMVTVSS

SEQ ID NO: 474 - protein, 107 aa, synthetic construct
DIQLTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIK

SEQ ID NO: 475 - protein, 121 aa, synthetic construct
EVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTTVTVSS

SEQ ID NO: 476 - protein, 107 aa, synthetic construct
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVDIK

SEQ ID NO: 477 - protein, 123 aa, synthetic construct
QVQLVESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSS

SEQ ID NO: 478 - protein, 123 aa, synthetic construct
EVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSS

SEQ ID NO: 479 - protein, 112 aa, synthetic construct
DIQLTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKVEIK

SEQ ID NO: 480 - protein, 122 aa, synthetic construct
EVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSS

SEQ ID NO: 481 - protein, 124 aa, synthetic construct
EVQLVESGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTTVTVSS

SEQ ID NO: 482 - protein, 121 aa, synthetic construct
EVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTMVTVSS

SEQ ID NO: 483 - protein, 123 aa, synthetic construct
EVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSS

SEQ ID NO: 484 - protein, 107 aa, synthetic construct
DIQLTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGRTKVEIK

SEQ ID NO: 485 - protein, 233 aa, synthetic construct
EVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPLLQGSG

SEQ ID NO: 486 - protein, 235 aa, synthetic construct
QITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPLLQGSG

SEQ ID NO: 487 - protein, 232 aa, synthetic construct
EVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPLLQGSG

SEQ ID NO: 488 - protein, 230 aa, synthetic construct
EVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPLLQGSG

SEQ ID NO: 489 - protein, 239 aa, synthetic construct
EVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPLLQGSG

SEQ ID NO: 490 - protein, 237 aa, synthetic construct
QVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPLLQGSG

SEQ ID NO: 491 - protein, 883 aa, Homo sapiens
AHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 492 - protein, 116 aa, synthetic construct
QVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSS

SEQ ID NO: 493 - protein, 105 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 494 - protein, 227 aa, synthetic construct
EVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 495 - protein, 229 aa, synthetic construct
QITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 496 - protein, 226 aa, synthetic construct
EVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 497 - protein, 224 aa, synthetic construct
EVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 498 - protein, 233 aa, synthetic construct
EVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 499 - protein, 231 aa, synthetic construct
QVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 500 - protein, 12 aa, synthetic construct
EQKLISEEDLGG

SEQ ID NO: 501 - protein, 6 aa, synthetic construct
HHHHHH

SEQ ID NO: 502 - protein, 233 aa, synthetic construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 503-506 - skipped (4 entries)

SEQ ID NO: 507 - protein, 22 aa, synthetic construct
LLNENSYVPREAGSQKDENLAL

SEQ ID NO: 508 - protein, 15 aa, synthetic construct
IYMDQTKFPIVNAEL

SEQ ID NO: 509 - protein, 5 aa, synthetic construct
TYKEL

SEQ ID NO: 510 - protein, 19 aa, synthetic construct
KRKLSEKLDSTDFTGTIKL

SEQ ID NO: 511 - protein, 20 aa, synthetic construct
YTLIEKTMQNVKHPVTGQFL

SEQ ID NO: 512 - protein, 17 aa, synthetic construct
LIERIPELNKVARAAAE

SEQ ID NO: 513 - protein, 19 aa, synthetic construct
LNENSYVPREAGSQKDENL

SEQ ID NO: 514 - protein, 9 aa, synthetic construct
GTKKDFEDL

SEQ ID NO: 515 - protein, 26 aa, synthetic construct
SVIIVDKNGRLVYLVENPGGYVAYSK

SEQ ID NO: 516 - protein, 19 aa, synthetic construct
LLNENSYVPREAGSQKDEN

SEQ ID NO: 517 - protein, 12 aa, synthetic construct
DQTKFPIVNAEL

SEQ ID NO: 518 - protein, 14 aa, synthetic construct
TYKELIERIPELNK

SEQ ID NO: 519 - protein, 24 aa, synthetic construct
SVIIVDKNGRLVYLVENPGGYVAY

SEQ ID NO: 520 - protein, 20 aa, synthetic construct
FGNMEGDCPSDWKTDSTCRM

SEQ ID NO: 521 - protein, 4 aa, synthetic construct
FEDL

SEQ ID NO: 522 - protein, 21 aa, synthetic construct
LVENPGGYVAYSKAATVTGKL

SEQ ID NO: 523 - protein, 17 aa, synthetic construct
IYMDQTKFPIVNAELSF

SEQ ID NO: 524 - protein, 9 aa, synthetic construct
ISRAAAEKL

SEQ ID NO: 525 - protein, 18 aa, synthetic construct
VTSESKNVKLTVSNVLKE

SEQ ID NO: 526 - protein, 16 aa, synthetic construct
FCEDTDYPYLGTTMDT

SEQ ID NO: 527 - protein, 112 aa, synthetic construct
DIQLTQSPLSLPVTPGEPASISCRSSQSLLHSNGYNYLDWYLQKPGQSPQFLIYLGSNRASGVPDRFSGSGSGTDFTLKINRVEAEDVGVYYCMQALQTPYTFGQGTKVEIK

SEQ ID NO: 528 - protein, 29 aa, Mus musculus
MHRPRRRGTRPPPLALLAALLLAARGADA

SEQ ID NO: 529 - protein, 4 aa, synthetic construct
LLNE

SEQ ID NO: 530 - protein, 8 aa, synthetic construct
DSTDFTGT

SEQ ID NO: 531 - protein, 9 aa, synthetic construct
VKHPVTGQF

SEQ ID NO: 532 - protein, 7 aa, synthetic construct
IERIPEL

SEQ ID NO: 533 - protein, 18 aa, synthetic construct
LNENSYVPREAGSQKDEN

SEQ ID NO: 534 - protein, 8 aa, synthetic construct
IVDKNGRL

SEQ ID NO: 535 - protein, 10 aa, synthetic construct
IVDKNGRLVY

SEQ ID NO: 536 - protein, 5 aa, synthetic construct
DQTKF

SEQ ID NO: 537 - protein, 8 aa, synthetic construct
LVENPGGY

SEQ ID NO: 538 - protein, 9 aa, synthetic construct
PIVNAELSF

SEQ ID NO: 539 - protein, 9 aa, synthetic construct
PYLGTTMDT

SEQ ID NO: 540 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 541 - protein, 15 aa, synthetic construct
GGGGSGGGGSGGGGS

SEQ ID NO: 542 - protein, 10 aa, synthetic construct
GGGGSGGGGS

SEQ ID NO: 543 - protein, 451 aa, synthetic construct
EVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 544 - protein, 451 aa, synthetic construct
EVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 545 - protein, 452 aa, synthetic construct
QVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 546 - protein, 453 aa, synthetic construct
EVQLVESGGGLVQPGRSLRLSCAASGFTFDDKAMHWVRQVPGKGLEWISGISWNSGTIGYADSVKGRFIISRDNAKNSLYLQMNSLRAEDTALYYCAKDGDTSGWYWYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 547 - protein, 450 aa, synthetic construct
QVQLVESGGGVVQPGRSLRLSCIASGFTFSVYGIHWVRQAPGKGLEWMAVISHDGNIKHYADSVKGRFTISRDNSKNTLYLQINSLRTEDTAVYYCAKDTWNSLDTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 548 - protein, 450 aa, synthetic construct
QVTLRESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 549 - protein, 446 aa, synthetic construct
EVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 550 - protein, 455 aa, synthetic construct
EVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 551 - protein, 457 aa, synthetic construct
EVQLVESGGGLVQPGRSLRLSCAASGFTFDDYAMHWVRQAPGKGLEWVSGISWNSGYIGYADSVKGRFTISRDNAENSLHLQMNSLRAEDTALYYCARGGSTLVRGVKGGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 552 - protein, 447 aa, synthetic construct
QVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 553 - protein, 455 aa, synthetic construct
EVQLVESGGGLVQPGGSLRLSCTASGFTFSNYWMTWVRQAPGKGLEWVANIKEDGSEKEYVDSVKGRFTISRDNAKNSLYLQMNSLRGEDTAVYYCARDGEQLVDYYYYYVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 554 - protein, 451 aa, synthetic construct
EVQLVESGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 555 - protein, 452 aa, synthetic construct
EVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 556 - protein, 454 aa, synthetic construct
QITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 557 - protein, 451 aa, synthetic construct
EVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 558 - protein, 446 aa, synthetic construct
QVQLVESGGGLVKPGGSLRLSCAASGFTFSDYFMSWIRQAPGKGLEWVSYISSTGSTINYADSVKGRFTISRDNVKNSLYLQMTSLRVEDTAVYYCTRDNWNYEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 559 - protein, 453 aa, synthetic construct
QLQLQESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 560 - protein, 448 aa, synthetic construct
QVQLVQSGAEVKKPGSSVRVSCKASRGTFSSYAISWVRQAPGQGLEWMGGIIPIFGTANYAQKFLARVTITADESTSTAYMELSSLRSEDTAVYYCAREKGWNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 561 - protein, 453 aa, synthetic construct
QVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 562 - protein, 452 aa, synthetic construct
QVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 563 - protein, 454 aa, synthetic construct
QVQLVQSGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 564 - protein, 448 aa, synthetic construct
EVQLVESGGGLIQPGGSLRLSCEASGFTFRNYEMNWVRQAPGKGLEWVSYISSSGNMKDYAESVKGRFTISRDNVKNSLQLQMNSLRVEDTAVYYCARDEFPYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 565 - protein, 451 aa, synthetic construct
EVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 566 - protein, 449 aa, synthetic construct
EVQLVESGGGLVQPGRSLTLSCAASGFTFDNFGMHWVRQGPGKGLEWVSGLTWNSGVIGYADSVKGRFTISRDNAKNSLYLQMNSLRPEDTALYYCAKDIRNYGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 567 - protein, 449 aa, synthetic construct
EVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 568 - protein, 451 aa, synthetic construct
EVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 569 - protein, 458 aa, synthetic construct
EVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 570 - protein, 456 aa, synthetic construct
QVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 571 - protein, 455 aa, synthetic construct
EVQLVESGGGLVQPGGSLRLSCAASGFTFSNYWMNWVRQAPGKGLEWVANIKEDGGKKLYVDSVKGRFTISRDNAKNSLFLQMNSLRAEDTAVYYCAREDTTLVVDYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 572 - protein, 452 aa, synthetic construct
QVQLVQSGAEVKKPGSSVKVSCKASGGTFNTYAITWVRQAPGQGLEWMGGIIPISGIAEYAQKFQGRVTITTDDSSTTAYMELNSLRSEDTAVYYCASWNYALYYFYGMDVWGRGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 573 - protein, 453 aa, synthetic construct
QVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 574 - protein, 452 aa, synthetic construct
EVQLVESGGGLVQPGRSLRLSCAVSGFTFDDYAMHWVRQAPGKGLEWVSGISWNSGTRGYADSVKGRFTISRDNAKNSLYLQMNSLRGEDTALYYCVKDITISPNYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 575 - protein, 330 aa, synthetic construct [sequence omitted]